SEQUENCE LISTING (306 sequences)

SEQ ID NO: 1 - DNA, 360 bp, synthetic construct
  misc_feature 1..360: note = Synthetic
  source 1..360: mol_type = other DNA
gaagtcaagcttcaggagtcaggacccggtcttgtagctcccagccaaagcctgtcagttacatgcacggtttccggtgtgtctttgccagattatggcgtatcttggattcgccaaccgcctagaaagggacttgagtggttgggtgtcatttggggatcagaaacaacttactataacagtgctcttaagtccaggttgactataatcaaggacaatagtaagtcccaagtttttctgaaaatgaattccctgcagacagatgacaccgctatctactactgtgccaagcactactattatgggggctcttatgctatggactattggggtcaggggacatcagttactgtttccagc

SEQ ID NO: 2 - protein, 276 aa, synthetic construct
  REGION 1..276: note = Synthetic
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 3 - DNA, 591 bp, synthetic construct
  misc_feature 1..591: note = Synthetic
  source 1..591: mol_type = other DNA
atgggaggccttgattacaaggacgacgacgacaaggctagcgatcaagtccaactggtggagtctggtggcgctttggtgcagccaggtggctctctgcgtttgtcctgtgccgcttctggcttcccagtgaaccgctattccatgcgctggtatcgccaggctccaggcaaagagcgtgagtgggtagccggtatgtccagcgcgggtgatcgtagctcctatgaagactccgtgaagggccgtttcaccatcagccgtgacgatgcccgtaacacggtgtatctgcaaatgaacagcttgaaacctgaagatacggccgtgtattactgtaatgtgaacgtgggcttcgagtattggggccaaggcacccaggtcaccgtgtccagcggcagtggtagtggctccggattcaatgtcttaatggttcatcggcgaagccatactggtgaacgcccattgcagtgcgaaatatgcggctttacctgccgccagcgcggtaacctcctccgccacattcgtctgcacacaggggaacggccttttcggtgtcacctctgcaactatgcatgccaaagaagagatgcgctc

SEQ ID NO: 4 - protein, 197 aa, synthetic construct
  REGION 1..197: note = Synthetic
MGGLDYKDDDDKASDQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGSGSGSGFNVLMVHRRSHTGERPLQCEICGFTCRQRGNLLRHIRLHTGERPFRCHLCNYACQRRDAL

SEQ ID NO: 5 - DNA, 939 bp, synthetic construct
  misc_feature 1..939: note = Synthetic
  source 1..939: mol_type = other DNA
atgggaggccttgattacaaggacgacgacgacaaggctagcgatcaagtccaactggtggagtctggtggcgctttggtgcagccaggtggctctctgcgtttgtcctgtgccgcttctggcttcccagtgaaccgctattccatgcgctggtatcgccaggctccaggcaaagagcgtgagtgggtagccggtatgtccagcgcgggtgatcgtagctcctatgaagactccgtgaagggccgtttcaccatcagccgtgacgatgcccgtaacacggtgtatctgcaaatgaacagcttgaaacctgaagatacggccgtgtattactgtaatgtgaacgtgggcttcgagtattggggccaaggcacccaggtcaccgtgtccagcggcagtggtagtggctccggaaggctcaattttggggacgatataccatcagcactccgcatagccaagaaaaaaagatggaactcaatcgaggaaaggagaatccatcaggaatctgaacttcatagctatttgtcccgactgattgcagcagagagagaaagggagcttgaggaatgtcaacgcaatcacgaaggggacgaggatgattctcacgtacgcgcacaacaagcctgtatcgaggccaaacatgataaatatatggcagatatggacgaactgttttcccaggtggacgagaaacgcaagaaaagagatattcctgactatctctgtggcaagatcagttttgaattgatgcgcgaaccatgtataacccccagtggcattacttatgaccgaaaggatattgaagaacatctgcaacgggtggggcactttgatccagttacacggtctccgctcacccaggaacagcttattccaaacttggccatgaaagaggtaatagacgcctttattagcgagaacggatgggtcgaggactac

SEQ ID NO: 6 - protein, 313 aa, synthetic construct
  REGION 1..313: note = Synthetic
MGGLDYKDDDDKASDQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGSGSGSGRLNFGDDIPSALRIAKKKRWNSIEERRIHQESELHSYLSRLIAAERERELEECQRNHEGDEDDSHVRAQQACIEAKHDKYMADMDELFSQVDEKRKKRDIPDYLCGKISFELMREPCITPSGITYDRKDIEEHLQRVGHFDPVTRSPLTQEQLIPNLAMKEVIDAFISENGWVEDY

SEQ ID NO: 7 - DNA, 465 bp, synthetic construct
  misc_feature 1..465: note = Synthetic
  source 1..465: mol_type = other DNA
atgggaggccttgattacaaggacgacgacgacaaggctagcgatcaagtccaactggtggagtctggtggcgctttggtgcagccaggtggctctctgcgtttgtcctgtgccgcttctggcttcccagtgaaccgctattccatgcgctggtatcgccaggctccaggcaaagagcgtgagtgggtagccggtatgtccagcgcgggtgatcgtagctcctatgaagactccgtgaagggccgtttcaccatcagccgtgacgatgcccgtaacacggtgtatctgcaaatgaacagcttgaaacctgaagatacggccgtgtattactgtaatgtgaacgtgggcttcgagtattggggccaaggcacccaggtcaccgtgtccagcggcagtggtagtggctccggaaacgcgcatcaaatattgcctaaggtcctgcataaacgaaccttgggtctttcc

SEQ ID NO: 8 - protein, 155 aa, synthetic construct
  REGION 1..155: note = Synthetic
MGGLDYKDDDDKASDQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGSGSGSGNAHQILPKVLHKRTLGLS

SEQ ID NO: 9 - DNA, 582 bp, synthetic construct
  misc_feature 1..582: note = Synthetic
  source 1..582: mol_type = other DNA
atgggaggccttgattacaaggacgacgacgacaaggctagcgatcaagtccaactggtggagtctggtggcgctttggtgcagccaggtggctctctgcgtttgtcctgtgccgcttctggcttcccagtgaaccgctattccatgcgctggtatcgccaggctccaggcaaagagcgtgagtgggtagccggtatgtccagcgcgggtgatcgtagctcctatgaagactccgtgaagggccgtttcaccatcagccgtgacgatgcccgtaacacggtgtatctgcaaatgaacagcttgaaacctgaagatacggccgtgtattactgtaatgtgaacgtgggcttcgagtattggggccaaggcacccaggtcaccgtgtccagcggcagtggtagtggctccggagctccacgaaacggcacggtccacctttacttgacgaaaccgctttatacctccgcaccgtccctccagcacctgtgtcggctgacaatcaataagtgtacaggtgcgatatggggattgccgcttccgacccgccttaaagattacctcgaggaatacaaatttcaagtc

SEQ ID NO: 10 - protein, 194 aa, synthetic construct
  REGION 1..194: note = Synthetic
MGGLDYKDDDDKASDQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGSGSGSGAPRNGTVHLYLTKPLYTSAPSLQHLCRLTINKCTGAIWGLPLPTRLKDYLEEYKFQV

SEQ ID NO: 11 - DNA, 1035 bp, synthetic construct
  misc_feature 1..1035: note = Synthetic
  source 1..1035: mol_type = other DNA
atgggaggccttgattacaaggacgacgacgacaaggctagcgatcaagtccaactggtggagtctggtggcgctttggtgcagccaggtggctctctgcgtttgtcctgtgccgcttctggcttcccagtgaaccgctattccatgcgctggtatcgccaggctccaggcaaagagcgtgagtgggtagccggtatgtccagcgcgggtgatcgtagctcctatgaagactccgtgaagggccgtttcaccatcagccgtgacgatgcccgtaacacggtgtatctgcaaatgaacagcttgaaacctgaagatacggccgtgtattactgtaatgtgaacgtgggcttcgagtattggggccaaggcacccaggtcaccgtgtccagcggcagtggtagtggctccggatccgtaaatatatctgggcaaaacacgatgaatatggtaaaggtccccgagtgcagattggccgatgagctggggggattgtgggagaacagccggttcaccgactgttgtctctgtgtagccggccaggagttccaagctcataaggcgattcttgctgcccgatctccagtattcagcgcgatgttcgaacatgagatggaagagtctaagaaaaacagggtcgagattaacgatgtagagcccgaagtctttaaggagatgatgtgtttcatatacacgggcaaggcgcctaatctggataagatggcggatgacttgctggcagcggcggataagtacgccttggaacgccttaaggtgatgtgtgaggacgctttgtgctccaatctgagcgtggagaacgcagcagaaatactcattctggcagacttgcattctgcggatcagttgaagactcaggccgtagatttcataaactatcacgcgtcagatgttctcgagacatctggctggaaatccatggtggtgtctcatcctcatttggttgcggaggcgtatcgctccctggcctctgcccaatgtcccttccttgggccgcctcggaaaaggctgaaacaatcc

SEQ ID NO: 12 - protein, 345 aa, synthetic construct
  REGION 1..345: note = Synthetic
MGGLDYKDDDDKASDQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGSGSGSGSVNISGQNTMNMVKVPECRLADELGGLWENSRFTDCCLCVAGQEFQAHKAILAARSPVFSAMFEHEMEESKKNRVEINDVEPEVFKEMMCFIYTGKAPNLDKMADDLLAAADKYALERLKVMCEDALCSNLSVENAAEILILADLHSADQLKTQAVDFINYHASDVLETSGWKSMVVSHPHLVAEAYRSLASAQCPFLGPPRKRLKQS

SEQ ID NO: 13 - DNA, 597 bp, synthetic construct
  misc_feature 1..597: note = Synthetic
  source 1..597: mol_type = other DNA
atgggaggccttgattacaaggacgacgacgacaaggctagcgatcaagtccaactggtggagtctggtggcgctttggtgcagccaggtggctctctgcgtttgtcctgtgccgcttctggcttcccagtgaaccgctattccatgcgctggtatcgccaggctccaggcaaagagcgtgagtgggtagccggtatgtccagcgcgggtgatcgtagctcctatgaagactccgtgaagggccgtttcaccatcagccgtgacgatgcccgtaacacggtgtatctgcaaatgaacagcttgaaacctgaagatacggccgtgtattactgtaatgtgaacgtgggcttcgagtattggggccaaggcacccaggtcaccgtgtccagcggcagtggtagtggctccggaacgctcccggtctacacgctcaaggagcgatgtctgcaagtggtacgaagcttggtcaagccggaaaactatcgccggctggatatagttcgatccctttatgaagaccttgaagaccaccctaatgttcaaaaggatcttgagcgattgactcaggaaagaatagctcaccaacgcatgggggat

SEQ ID NO: 14 - protein, 199 aa, synthetic construct
  REGION 1..199: note = Synthetic
MGGLDYKDDDDKASDQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGSGSGSGTLPVYTLKERCLQVVRSLVKPENYRRLDIVRSLYEDLEDHPNVQKDLERLTQERIAHQRMGD

SEQ ID NO: 15 - DNA, 696 bp, synthetic construct
  misc_feature 1..696: note = Synthetic
  source 1..696: mol_type = other DNA
atgggaggccttgattacaaggacgacgacgacaaggctagcgatcaagtccaactggtggagtctggtggcgctttggtgcagccaggtggctctctgcgtttgtcctgtgccgcttctggcttcccagtgaaccgctattccatgcgctggtatcgccaggctccaggcaaagagcgtgagtgggtagccggtatgtccagcgcgggtgatcgtagctcctatgaagactccgtgaagggccgtttcaccatcagccgtgacgatgcccgtaacacggtgtatctgcaaatgaacagcttgaaacctgaagatacggccgtgtattactgtaatgtgaacgtgggcttcgagtattggggccaaggcacccaggtcaccgtgtccagcggcagtggtagtggctccggagagcaagcaccggaagatcagggaccacagcgcgaaccacacaatgaatggacattggaattgcttgaggagcttaaaaatgaagctgttagacacttcccaagaatctggctgcacggcttgggtcagcacatatatgaaacgtatggagatacatgggcaggtgtcgaggctatcatcaggatccttcagcagctcctcttcattcatttcagaatagggtgtcgacatagcagaataggggttacccagcaacgcagggcgcggaatggtgccagtaggagt

SEQ ID NO: 16 - protein, 232 aa, synthetic construct
  REGION 1..232: note = Synthetic
MGGLDYKDDDDKASDQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGSGSGSGEQAPEDQGPQREPHNEWTLELLEELKNEAVRHFPRIWLHGLGQHIYETYGDTWAGVEAIIRILQQLLFIHFRIGCRHSRIGVTQQRRARNGASRS

SEQ ID NO: 17 - DNA, 744 bp, synthetic construct
  misc_feature 1..744: note = Synthetic
  source 1..744: mol_type = other DNA
atgggaggccttgattacaaggacgacgacgacaaggctagcgatcaagtccaactggtggagtctggtggcgctttggtgcagccaggtggctctctgcgtttgtcctgtgccgcttctggcttcccagtgaaccgctattccatgcgctggtatcgccaggctccaggcaaagagcgtgagtgggtagccggtatgtccagcgcgggtgatcgtagctcctatgaagactccgtgaagggccgtttcaccatcagccgtgacgatgcccgtaacacggtgtatctgcaaatgaacagcttgaaacctgaagatacggccgtgtattactgtaatgtgaacgtgggcttcgagtattggggccaaggcacccaggtcaccgtgtccagcggcagtggtagtggctccggagcagacccgagggaaagagttccaccaggtaacagtggcgaagaaacgatcggcgaagcgttcgagtggcttgatcggactattgaggctcttaatcgggaggctgtgaaccacctgccccgggaacttattttccaggtctggcagagaagttgggcctactggcacgatgagcaaggcatgtccacatcctatacaaagtatcggtatctttgtataatgcagaaggctgtatatatccactttaagaagggatgtacttgtctgggtagaggtcatggaccaggagggtggagaccaggccctccacctcctccacctccaggtcttgtg

SEQ ID NO: 18 - protein, 248 aa, synthetic construct
  REGION 1..248: note = Synthetic
MGGLDYKDDDDKASDQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGSGSGSGADPRERVPPGNSGEETIGEAFEWLDRTIEALNREAVNHLPRELIFQVWQRSWAYWHDEQGMSTSYTKYRYLCIMQKAVYIHFKKGCTCLGRGHGPGGWRPGPPPPPPPGLV

SEQ ID NO: 19 - DNA, 513 bp, synthetic construct
  misc_feature 1..513: note = Synthetic
  source 1..513: mol_type = other DNA
atgggaggccttgattacaaggacgacgacgacaaggctagcgatcaagtccaactggtggagtctggtggcgctttggtgcagccaggtggctctctgcgtttgtcctgtgccgcttctggcttcccagtgaaccgctattccatgcgctggtatcgccaggctccaggcaaagagcgtgagtgggtagccggtatgtccagcgcgggtgatcgtagctcctatgaagactccgtgaagggccgtttcaccatcagccgtgacgatgcccgtaacacggtgtatctgcaaatgaacagcttgaaacctgaagatacggccgtgtattactgtaatgtgaacgtgggcttcgagtattggggccaaggcacccaggtcaccgtgtccagcggcagtggtagtggctccggaggaccgtgttgcttggacgtaacgtgtgcagaattgcgcacaatggattcaacagtgaacttcgtgtcatggcatgccaatcggcaattggggatgccaagc

SEQ ID NO: 20 - protein, 171 aa, synthetic construct
  REGION 1..171: note = Synthetic
MGGLDYKDDDDKASDQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGSGSGSGGPCCLDVTCAELRTMDSTVNFVSWHANRQLGMPS

SEQ ID NO: 21 - DNA, 594 bp, synthetic construct
  misc_feature 1..594: note = Synthetic
  source 1..594: mol_type = other DNA
atgggaggccttgattacaaggacgacgacgacaaggctagcttcaatgtcttaatggttcatcggcgaagccatactggtgaacgcccattgcagtgcgaaatatgcggctttacctgccgccagcgcggtaacctcctccgccacattcgtctgcacacaggggaacggccttttcggtgtcacctctgcaactatgcatgccaaagaagagatgcgctcagcgggtcaggtagtggctccggagatcaagtccaactggtggagtctggtggcgctttggtgcagccaggtggctctctgcgtttgtcctgtgccgcttctggcttcccagtgaaccgctattccatgcgctggtatcgccaggctccaggcaaagagcgtgagtgggtagccggtatgtccagcgcgggtgatcgtagctcctatgaagactccgtgaagggccgtttcaccatcagccgtgacgatgcccgtaacacggtgtatctgcaaatgaacagcttgaaacctgaagatacggccgtgtattactgtaatgtgaacgtgggcttcgagtattggggccaaggcacccaggtcaccgtgtccagc

SEQ ID NO: 22 - protein, 198 aa, synthetic construct
  REGION 1..198: note = Synthetic
MGGLDYKDDDDKASFNVLMVHRRSHTGERPLQCEICGFTCRQRGNLLRHIRLHTGERPFRCHLCNYACQRRDALSGSGSGSGDQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSS

SEQ ID NO: 23 - DNA, 1200 bp, synthetic construct
  misc_feature 1..1200: note = Synthetic
  source 1..1200: mol_type = other DNA
atgggaggccttgattacaaggacgacgacgacaaggctagcgaccctgctgaagccgtgctgcaggagaaggcactcaagtttatgaatagctcagaaagggaagactgtaataacggagagcctccgaggaaaattatacccgaaaagaacagtctgcggcaaacttacaatagttgtgccagactttgtttgaaccaggaaaccgtatgtttggcgtctaccgcaatgaagacagagaattgcgtggcgaagaccaaactcgctaacggaacatcaagcatgatagtccccaaacagagaaagcttagtgcgtcttacgaaaaggagaaggagctgtgtgtcaagtatttcgagcagtggagtgagagcgaccaggtagaattcgtggagcacctcatcagccagatgtgccattatcagcatggccacataaacagctacttgaagcccatgcttcagcgagactttattactgcattgcctgctagaggtctggatcacattgcggaaaatatcttgtcctatttggatgctaagagcctttgcgcggccgaactggtatgcaaagaatggtatagggtgacgtctgacggaatgttgtggaagaaactcattgagagaatggtgcgcacggactctctctggagagggctggcggagcgcagaggttggggacagtatttgttcaagaataaaccgcctgacgggaacgctccaccgaacagtttttatcgcgctctgtatccaaaaataatacaagatatcgaaacaattgagtctaactggcgatgcggcaggcacagtctgcagcgcatccattgccgaagcgggtcaggtagtggctccggagatcaagtccaactggtggagtctggtggcgctttggtgcagccaggtggctctctgcgtttgtcctgtgccgcttctggcttcccagtgaaccgctattccatgcgctggtatcgccaggctccaggcaaagagcgtgagtgggtagccggtatgtccagcgcgggtgatcgtagctcctatgaagactccgtgaagggccgtttcaccatcagccgtgacgatgcccgtaacacggtgtatctgcaaatgaacagcttgaaacctgaagatacggccgtgtattactgtaatgtgaacgtgggcttcgagtattggggccaaggcacccaggtcaccgtgtccagc

SEQ ID NO: 24 - protein, 400 aa, synthetic construct
  REGION 1..400: note = Synthetic
MGGLDYKDDDDKASDPAEAVLQEKALKFMNSSEREDCNNGEPPRKIIPEKNSLRQTYNSCARLCLNQETVCLASTAMKTENCVAKTKLANGTSSMIVPKQRKLSASYEKEKELCVKYFEQWSESDQVEFVEHLISQMCHYQHGHINSYLKPMLQRDFITALPARGLDHIAENILSYLDAKSLCAAELVCKEWYRVTSDGMLWKKLIERMVRTDSLWRGLAERRGWGQYLFKNKPPDGNAPPNSFYRALYPKIIQDIETIESNWRCGRHSLQRIHCRSGSGSGSGDQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSS

SEQ ID NO: 25 - DNA, 1290 bp, synthetic construct
  misc_feature 1..1290: note = Synthetic
  source 1..1290: mol_type = other DNA
atgggaggccttgattacaaggacgacgacgacaaggctagctgcgtaccgagatctggactgatattgagttgtatctgcctttactgtggcgtactgctccccgttttgttgccgaatctcccgtttctgacatgtctctctatgtcaaccttggagagcgtgacctacctgccagagaaaggtctgtactgtcagcgcttgccctcatccaggacacatggcggaacagaatctctgaaagggaagaatacggagaatatgggattttacggaacgttgaaaatgattttctacaagatgaaacggaaactcgatcacggaagcgaagtacggtcattttctcttgggaagaagccttgtaaagtgtccgagtatacatcaacgacggggctggtcccttgtagtgccacacctaccaccttcggtgatcttagagcagctaacggtcaaggccaacaaaggcggcgcatcacgtctgttcaaccacctaccggtcttcaggaatggcttaagatgttccagtcttggtctggacccgagaaattgcttgccctcgacgagctgattgacagttgtgaaccaacacaagttaaacacatgatgcaagttatcgagcctcagtttcaacgagactttatttctttgcttccgaaggaattggctctgtacgtgctttcatttcttgaacccaaggacctcctgcaagccgcccaaacgtgccgctattggcgcatccttgcggaagataatttgctctggagggaaaagtgtaaggaggaaggaattgatgaaccactgcatattaaacggcgcaaggtcattaaaccaggatttatacattcaccttggaagtctgcatacatacgccagcatcgcatcgacactaactggcgacgaggcgagttgaaatccccgagcgggtcaggtagtggctccggagatcaagtccaactggtggagtctggtggcgctttggtgcagccaggtggctctctgcgtttgtcctgtgccgcttctggcttcccagtgaaccgctattccatgcgctggtatcgccaggctccaggcaaagagcgtgagtgggtagccggtatgtccagcgcgggtgatcgtagctcctatgaagactccgtgaagggccgtttcaccatcagccgtgacgatgcccgtaacacggtgtatctgcaaatgaacagcttgaaacctgaagatacggccgtgtattactgtaatgtgaacgtgggcttcgagtattggggccaaggcacccaggtcaccgtgtccagc

SEQ ID NO: 26 - protein, 430 aa, synthetic construct
  REGION 1..430: note = Synthetic
MGGLDYKDDDDKASCVPRSGLILSCICLYCGVLLPVLLPNLPFLTCLSMSTLESVTYLPEKGLYCQRLPSSRTHGGTESLKGKNTENMGFYGTLKMIFYKMKRKLDHGSEVRSFSLGKKPCKVSEYTSTTGLVPCSATPTTFGDLRAANGQGQQRRRITSVQPPTGLQEWLKMFQSWSGPEKLLALDELIDSCEPTQVKHMMQVIEPQFQRDFISLLPKELALYVLSFLEPKDLLQAAQTCRYWRILAEDNLLWREKCKEEGIDEPLHIKRRKVIKPGFIHSPWKSAYIRQHRIDTNWRRGELKSPSGSGSGSGDQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSS

SEQ ID NO: 27 - DNA, 426 bp, synthetic construct
  misc_feature 1..426: note = Synthetic
  source 1..426: mol_type = other DNA
atgaacgcgcatcaaatattgcctaaggtcctgcataaacgaaccttgggtcttagcgggtcaggtagtggctccggagatcaagtccaactggtggagtctggtggcgctttggtgcagccaggtggctctctgcgtttgtcctgtgccgcttctggcttcccagtgaaccgctattccatgcgctggtatcgccaggctccaggcaaagagcgtgagtgggtagccggtatgtccagcgcgggtgatcgtagctcctatgaagactccgtgaagggccgtttcaccatcagccgtgacgatgcccgtaacacggtgtatctgcaaatgaacagcttgaaacctgaagatacggccgtgtattactgtaatgtgaacgtgggcttcgagtattggggccaaggcacccaggtcaccgtgtccagc

SEQ ID NO: 28 - protein, 142 aa, synthetic construct
  REGION 1..142: note = Synthetic
MNAHQILPKVLHKRTLGLSGSGSGSGDQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSS

SEQ ID NO: 29 - DNA, 819 bp, synthetic construct
  misc_feature 1..819: note = Synthetic
  source 1..819: mol_type = other DNA
atgcatcgaaagcatcttcaggagatacctgacttgtcatccaatgtagccacgtccttcacctggggctgggacagcagcaagacctccgaacttctgtccggaatgggggtctctgctctggagaaggaggagcctgacagcgaaaatatcccccaggaattgctctcaaaccttggtcatcccgagtcacctcctcggaagcgcttgaagtctaagggctccgacaaggacttcgtaatagtaaggaggcccaaacttaaccgagagaactttcctggggtcagttgggatagtctgccggacgagctgctcctcgggatattctcctgcttgtgtctgccggaacttcttaaggtttctggtgtctgtaagcgctggtatcgcctcgcttctgacgagagcctttggcagacgttggaccttaccgggaaaaaccttggttccgcagggtcagcagccggatctggagagtttgtccaactggtggagtctggtggcgctttggtgcagccaggtggctctctgcgtttgtcctgtgccgcttctggcttcccagtgaaccgctattccatgcgctggtatcgccaggctccaggcaaagagcgtgagtgggtagccggtatgtccagcgcgggtgatcgtagctcctatgaagactccgtgaagggccgtttcaccatcagccgtgacgatgcccgtaacacggtgtatctgcaaatgaacagcttgaaacctgaagatacggccgtgtattactgtaatgtgaacgtgggcttcgagtattggggccaaggcacccaggtcaccgtctccagc

SEQ ID NO: 30 - protein, 273 aa, synthetic construct
  REGION 1..273: note = Synthetic
MHRKHLQEIPDLSSNVATSFTWGWDSSKTSELLSGMGVSALEKEEPDSENIPQELLSNLGHPESPPRKRLKSKGSDKDFVIVRRPKLNRENFPGVSWDSLPDELLLGIFSCLCLPELLKVSGVCKRWYRLASDESLWQTLDLTGKNLGSAGSAAGSGEFVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSS

SEQ ID NO: 31 - DNA, 696 bp, synthetic construct
  misc_feature 1..696: note = Synthetic
  source 1..696: mol_type = other DNA
atgggaggccttgattacaaggacgacgacgacaaggctagcgagcaagcaccggaagatcagggaccacagcgcgaaccacacaatgaatggacattggaattgcttgaggagcttaaaaatgaagctgttagacacttcccaagaatctggctgcacggcttgggtcagcacatatatgaaacgtatggagatacatgggcaggtgtcgaggctatcatcaggatccttcagcagctcctcttcattcatttcagaatagggtgtcgacatagcagaataggggttacccagcaacgcagggcgcggaatggtgccagtaggagcgggtcaggtagtggctccggagatcaagtccaactggtggagtctggtggcgctttggtgcagccaggtggctctctgcgtttgtcctgtgccgcttctggcttcccagtgaaccgctattccatgcgctggtatcgccaggctccaggcaaagagcgtgagtgggtagccggtatgtccagcgcgggtgatcgtagctcctatgaagactccgtgaagggccgtttcaccatcagccgtgacgatgcccgtaacacggtgtatctgcaaatgaacagcttgaaacctgaagatacggccgtgtattactgtaatgtgaacgtgggcttcgagtattggggccaaggcacccaggtcaccgtgtccagc

SEQ ID NO: 32 - protein, 232 aa, synthetic construct
  REGION 1..232: note = Synthetic
MGGLDYKDDDDKASEQAPEDQGPQREPHNEWTLELLEELKNEAVRHFPRIWLHGLGQHIYETYGDTWAGVEAIIRILQQLLFIHFRIGCRHSRIGVTQQRRARNGASRSGSGSGSGDQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSS

SEQ ID NO: 33 - DNA, 714 bp, synthetic construct
  misc_feature 1..714: note = Synthetic
  source 1..714: mol_type = other DNA
atggcagacccgagggaaagagttccaccaggtaacagtggcgaagaaacgatcggcgaagcgttcgagtggcttgatcggactattgaggctcttaatcgggaggctgtgaaccacctgccccgggaacttattttccaggtctggcagagaagttgggcctactggcacgatgagcaaggcatgtccacatcctatacaaagtatcggtatctttgtataatgcagaaggctgtatatatccactttaagaagggatgtacttgtctgggtagaggtcatggaccaggagggtggagaccaggccctccacctcctccacctccaggtcttgtgggttccgcagggtcagcagccggatctggagagtttgtccaactggtggagtctggtggcgctttggtgcagccaggtggctctctgcgtttgtcctgtgccgcttctggcttcccagtgaaccgctattccatgcgctggtatcgccaggctccaggcaaagagcgtgagtgggtagccggtatgtccagcgcgggtgatcgtagctcctatgaagactccgtgaagggccgtttcaccatcagccgtgacgatgcccgtaacacggtgtatctgcaaatgaacagcttgaaacctgaagatacggccgtgtattactgtaatgtgaacgtgggcttcgagtattggggccaaggcacccaggtcaccgtctccagc

SEQ ID NO: 34 - protein, 238 aa, synthetic construct
  REGION 1..238: note = Synthetic
MADPRERVPPGNSGEETIGEAFEWLDRTIEALNREAVNHLPRELIFQVWQRSWAYWHDEQGMSTSYTKYRYLCIMQKAVYIHFKKGCTCLGRGHGPGGWRPGPPPPPPPGLVGSAGSAAGSGEFVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSS

SEQ ID NO: 35 - DNA, 483 bp, synthetic construct
  misc_feature 1..483: note = Synthetic
  source 1..483: mol_type = other DNA
atgggaccgtgttgcttggacgtaacgtgtgcagaattgcgcacaatggattcaacagtgaacttcgtctcatggcatgccaatcggcaattggggatgccaagcggttccgcagggtcagcagccggatctggagagtttgtccaactggtggagtctggtggcgctttggtgcagccaggtggctctctgcgtttgtcctgtgccgcttctggcttcccagtgaaccgctattccatgcgctggtatcgccaggctccaggcaaagagcgtgagtgggtagccggtatgtccagcgcgggtgatcgtagctcctatgaagactccgtgaagggccgtttcaccatcagccgtgacgatgcccgtaacacggtgtatctgcaaatgaacagcttgaaacctgaagatacggccgtgtattactgtaatgtgaacgtgggcttcgagtattggggccaaggcacccaggtcaccgtctccagc

SEQ ID NO: 36 - protein, 161 aa, synthetic construct
  REGION 1..161: note = Synthetic
MGPCCLDVTCAELRTMDSTVNFVSWHANRQLGMPSGSAGSAAGSGEFVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSS

SEQ ID NO: 37 - DNA, 864 bp, synthetic construct
  misc_feature 1..864: note = Synthetic
  source 1..864: mol_type = other DNA
atgcaaatacctcaagcgccctggcccgtagtgtgggcagtcctccaacttggctggaggccaggatggttcttggacagccctgaccgcccttggaatcccccgactttttcaccagcgctgctcgttgtcacagaaggagacaatgctacgtttacttgttctttctctaacacttcagaatcatttgttctcaattggtataggatgagtccctctaatcagacagataaactggcggcatttccagaggatcgcagtcagccggggcaggactgccgatttagggttacccagctccccaatgggcgagactttcacatgtctgtggtccgggcccgcagaaatgactctggtacttatctctgtggggctataagcttggcgccgaaagcacaaataaaagaaagcttgcgcgcggaactgcgcgttacagaaagaagggcagaggtgcccacagctcatccgagcccctctccgcggccagcgggacagtttcaaacactggtcgtaggagtagtaggcggtttgctgggatcattggtacttcttgtatgggtgttggccgtaatatgcagccgagctgcgagaggaacaattggcgcaagaaggaccgggcagccactgaaagaggacccatcagccgtaccggtgttttcagttgactacggagaattggacttccagtggcgcgagaaaaccccagaaccacctgttccctgtgttcctgaacaaactgagtacgcaactatcgtgtttccatcaggcatgggtacttcttccccggcccgaagaggttccgcagacggacccaggtctgcacagccccttcgcccggaggacggtcattgttcctggccactg

SEQ ID NO: 38 - protein, 288 aa, synthetic construct
  REGION 1..288: note = Synthetic
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 39 - DNA, 666 bp, synthetic construct
  misc_feature 1..666: note = Synthetic
  source 1..666: mol_type = other DNA
atgggttgcatcaagagcaagcggaaagataacctcaacgatgatggggttgacatgaaaacaggcagtggctcgggctcggggtccggcggacttgattacaaggacgacgacgacaaggctagcgatcaagtccaactggtggagtctggtggcgctttggtgcagccaggtggctctctgcgtttgtcctgtgccgcttctggcttcccagtgaaccgctattccatgcgctggtatcgccaggctccaggcaaagagcgtgagtgggtagccggtatgtccagcgcgggtgatcgtagctcctatgaagactccgtgaagggccgtttcaccatcagccgtgacgatgcccgtaacacggtgtatctgcaaatgaacagcttgaaacctgaagatacggccgtgtattactgtaatgtgaacgtgggcttcgagtattggggccaaggcacccaggtcaccgtgtccagcggcagtggtagtggctccggagctccacgaaacggcacggtccacctttacttgacgaaaccgctttatacctccgcaccgtccctccagcacctgtgtcggctgacaatcaataagtgtacaggtgcgatatggggattgccgcttccgacccgccttaaagattacctcgaggaatacaaatttcaagtc

SEQ ID NO: 40 - protein, 222 aa, synthetic construct
  REGION 1..222: note = Synthetic
MGCIKSKRKDNLNDDGVDMKTGSGSGSGSGGLDYKDDDDKASDQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGSGSGSGAPRNGTVHLYLTKPLYTSAPSLQHLCRLTINKCTGAIWGLPLPTRLKDYLEEYKFQV

SEQ ID NO: 41 - DNA, 828 bp, synthetic construct
  misc_feature 1..828: note = Synthetic
  source 1..828: mol_type = other DNA
atgggttgcatcaagagcaagcggaaagataacctcaacgatgatggggttgacatgaaaacaggcagtggctcgggctcggggtccggcggacttgattacaaggacgacgacgacaaggctagcgatcaagtccaactggtggagtctggtggcgctttggtgcagccaggtggctctctgcgtttgtcctgtgccgcttctggcttcccagtgaaccgctattccatgcgctggtatcgccaggctccaggcaaagagcgtgagtgggtagccggtatgtccagcgcgggtgatcgtagctcctatgaagactccgtgaagggccgtttcaccatcagccgtgacgatgcccgtaacacggtgtatctgcaaatgaacagcttgaaacctgaagatacggccgtgtattactgtaatgtgaacgtgggcttcgagtattggggccaaggcacccaggtcaccgtgtccagcggcagtggtagtggctccggagcagacccgagggaaagagttccaccaggtaacagtggcgaagaaacgatcggcgaagcgttcgagtggcttgatcggactattgaggctcttaatcgggaggctgtgaaccacctgccccgggaacttattttccaggtctggcagagaagttgggcctactggcacgatgagcaaggcatgtccacatcctatacaaagtatcggtatctttgtataatgcagaaggctgtatatatccactttaagaagggatgtacttgtctgggtagaggtcatggaccaggagggtggagaccaggccctccacctcctccacctccaggtcttgtg

SEQ ID NO: 42 - protein, 276 aa, synthetic construct
  REGION 1..276: note = Synthetic
MGCIKSKRKDNLNDDGVDMKTGSGSGSGSGGLDYKDDDDKASDQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGSGSGSGADPRERVPPGNSGEETIGEAFEWLDRTIEALNREAVNHLPRELIFQVWQRSWAYWHDEQGMSTSYTKYRYLCIMQKAVYIHFKKGCTCLGRGHGPGGWRPGPPPPPPPGLV

SEQ ID NO: 43 - DNA, 678 bp, synthetic construct
  misc_feature 1..678: note = Synthetic
  source 1..678: mol_type = other DNA
atgggttgcatcaagagcaagcggaaagataacctcaacgatgatggggttgacatgaaaacaggcagtggctcgggctcggggtccggcggacttgattacaaggacgacgacgacaaggctagcttcaatgtcttaatggttcatcggcgaagccatactggtgaacgcccattgcagtgcgaaatatgcggctttacctgccgccagcgcggtaacctcctccgccacattcgtctgcacacaggggaacggccttttcggtgtcacctctgcaactatgcatgccaaagaagagatgcgctcagcgggtcaggtagtggctccggagatcaagtccaactggtggagtctggtggcgctttggtgcagccaggtggctctctgcgtttgtcctgtgccgcttctggcttcccagtgaaccgctattccatgcgctggtatcgccaggctccaggcaaagagcgtgagtgggtagccggtatgtccagcgcgggtgatcgtagctcctatgaagactccgtgaagggccgtttcaccatcagccgtgacgatgcccgtaacacggtgtatctgcaaatgaacagcttgaaacctgaagatacggccgtgtattactgtaatgtgaacgtgggcttcgagtattggggccaaggcacccaggtcaccgtgtccagc

SEQ ID NO: 44 - protein, 226 aa, synthetic construct
  REGION 1..226: note = Synthetic
MGCIKSKRKDNLNDDGVDMKTGSGSGSGSGGLDYKDDDDKASFNVLMVHRRSHTGERPLQCEICGFTCRQRGNLLRHIRLHTGERPFRCHLCNYACQRRDALSGSGSGSGDQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSS

SEQ ID NO: 45 - DNA, 2658 bp, synthetic construct
  misc_feature 1..2658: note = Synthetic
  source 1..2658: mol_type = other DNA
aagggcggcggcggctccggcggcggcggctccgacttcccttcctgcaggtgcgttgaacaaataattgaaaaagacgagggtccattttatacacatctgggagctggaccaaacgtcgcggcaatccgcgagataatggaagaacggttcggccaaaaagggaaagcaataagaatcgaacgcgttatttacactggtaaggagggtaagagtagccaggggtgcccaatagctaaatgggtcgtcagaaggtcctcaagtgaagagaaactcctctgccttgtccgagaaagggctggccacacttgtgaggcagccgtcatcgtcatcttgattctggtctgggaggggatcccccttagtctcgcagacaaactctattccgagctcaccgaaacgcttcgcaaatatgggactctgacaaataggaggtgcgccctgaatgaagagagaacatgcgcatgccaaggcttggatcccgagacctgtggtgccagcttttcctttggatgtagctggagtatgtactacaacggctgtaagtttgcaaggtccaaaattcctcgcaaatttaagttgctcggcgacgaccctaaggaagaagaaaagctcgagagtcatctccaaaacttgagtactctcatggcccctacatataagaaactggctccggatgcatataacaatcagatagaatatgagcacagggcacccgagtgcagactgggattgaaggaagggaggcctttttcaggtgtaacggcttgcctcgacttttgtgctcacgctcacagggatctgcacaacatgcagaacggcagcacactcgtttgtactctgaccagagaagacaatcgagagttcggtgggaaacctgaggacgaacaacttcacgtcttgccactgtacaaagtctctgatgttgatgaatttgggtcagttgaagcccaagaggaaaaaaaacgatcaggggcgatccaagtactgtcatcatttcggagaaaggtaagaatgcttgccgagcctgtcaaaacgtgccgccaacgcaaactggaggccaaaaaggcagcagcagaaaagttgagttctcttgagaacagctcaaacaaaaacgagaaagaaaagagcgcaccgtcacggacaaagcagacagaaaacgcttcacaagcgaagcagctcgcagaactccttcggttgtcaggccccgtcatgcaacaaagtcaacagccccagccgctgcagaaacagccaccccagccccaacagcaacagagaccgcaacaacaacagccccatcacccgcagacagaaagcgtaaatagctattccgcgtcagggtccaccaacccttatatgcggaggcccaatcctgtgagtccatatcccaattccagtcacacttccgatatctatgggagcacaagccccatgaatttttactctacttcatctcaggccgcaggttcatatcttaatagctccaatccgatgaatccctatcccggcctccttaatcagaatacccagtacccatcataccaatgcaacggaaacctgtcagttgataactgctccccgtaccttggaagctactccccacagtcccagcccatggatctgtaccggtatccaagtcaggacccactgtctaaattgagtctgccgccaattcacactctttatcaaccgagatttggaaacagccagtcattcacatctaaatacctgggttatggaaatcagaatatgcaaggagatggattctcctcatgtaccattcgaccaaacgttcaccatgttggcaagctcccaccttaccccacccatgaaatggatggtcatttcatgggagcgacctctcgattgccgccaaatctgagtaatcctaacatggattataaaaatggtgaacaccatagcccttcccatattatccataactattctgcagccccagggatgtttaactcttcattgcacgctttgcatttgcagaacaaggagaatgatatgctgtcccataccgcaaacggactcagtaaaatgcttccagcgcttaatcatgaccggacagcgtgcgtccaaggtgggcttcacaagctttccgacgccaacggtcaggagaagcagcctctggctttggtccagggggtggcgtccggagcagaagacaacgacgaagtatggagcgatagcgagcagagcttcctggatcctgacattgggggcgtggccgtggcaccgacccatggctctatccttatagagtgcgcgaaacgagagctccacgccaccacaccgcttaagaatcccaatcgcaatcatccaacacgcatcagtcttgtattttatcaacacaagtccatgaatgagcccaagcacggtctggccctttgggaagctaaaatggctgaaaaagcgagggagaaggaagaagaatgcgaaaagtatgggcccgattacgttcctcaaaaaagtcatggaaaaaaagtcaagcgcgagccagctgaacctcacgagacttcagaacctacgtacttgagattcattaagtctcttgctgaacggaccatgtctgtaaccacagactcaacggttaccacctctccatatgccttcacgagagttactggcccatataaccggtatatataa

SEQ ID NO: 46 - protein, 885 aa, synthetic construct
  REGION 1..885: note = Synthetic
KGGGGSGGGGSDFPSCRCVEQIIEKDEGPFYTHLGAGPNVAAIREIMEERFGQKGKAIRIERVIYTGKEGKSSQGCPIAKWVVRRSSSEEKLLCLVRERAGHTCEAAVIVILILVWEGIPLSLADKLYSELTETLRKYGTLTNRRCALNEERTCACQGLDPETCGASFSFGCSWSMYYNGCKFARSKIPRKFKLLGDDPKEEEKLESHLQNLSTLMAPTYKKLAPDAYNNQIEYEHRAPECRLGLKEGRPFSGVTACLDFCAHAHRDLHNMQNGSTLVCTLTREDNREFGGKPEDEQLHVLPLYKVSDVDEFGSVEAQEEKKRSGAIQVLSSFRRKVRMLAEPVKTCRQRKLEAKKAAAEKLSSLENSSNKNEKEKSAPSRTKQTENASQAKQLAELLRLSGPVMQQSQQPQPLQKQPPQPQQQQRPQQQQPHHPQTESVNSYSASGSTNPYMRRPNPVSPYPNSSHTSDIYGSTSPMNFYSTSSQAAGSYLNSSNPMNPYPGLLNQNTQYPSYQCNGNLSVDNCSPYLGSYSPQSQPMDLYRYPSQDPLSKLSLPPIHTLYQPRFGNSQSFTSKYLGYGNQNMQGDGFSSCTIRPNVHHVGKLPPYPTHEMDGHFMGATSRLPPNLSNPNMDYKNGEHHSPSHIIHNYSAAPGMFNSSLHALHLQNKENDMLSHTANGLSKMLPALNHDRTACVQGGLHKLSDANGQEKQPLALVQGVASGAEDNDEVWSDSEQSFLDPDIGGVAVAPTHGSILIECAKRELHATTPLKNPNRNHPTRISLVFYQHKSMNEPKHGLALWEAKMAEKAREKEEECEKYGPDYVPQKSHGKKVKREPAEPHETSEPTYLRFIKSLAERTMSVTTDSTVTTSPYAFTRVTGPYNRYI

SEQ ID NO: 47 - DNA, 2664 bp, synthetic construct
  misc_feature 1..2664: note = Synthetic
  source 1..2664: mol_type = other DNA
atgggaggccttgattacaaggacgacgacgacaaggctagcgacttcccttcctgcaggtgcgttgaacaaataattgaaaaagacgagggtccattttatacacatctgggagctggaccaaacgtcgcggcaatccgcgagataatggaagaacggttcggccaaaaagggaaagcaataagaatcgaacgcgttatttacactggtaaggagggtaagagtagccaggggtgcccaatagctaaatgggtcgtcagaaggtcctcaagtgaagagaaactcctctgccttgtccgagaaagggctggccacacttgtgaggcagccgtcatcgtcatcttgattctggtctgggaggggatcccccttagtctcgcagacaaactctattccgagctcaccgaaacgcttcgcaaatatgggactctgacaaataggaggtgcgccctgaatgaagagagaacatgcgcatgccaaggcttggatcccgagacctgtggtgccagcttttcctttggatgtagctggagtatgtactacaacggctgtaagtttgcaaggtccaaaattcctcgcaaatttaagttgctcggcgacgaccctaaggaagaagaaaagctcgagagtcatctccaaaacttgagtactctcatggcccctacatataagaaactggctccggatgcatataacaatcagatagaatatgagcacagggcacccgagtgcagactgggattgaaggaagggaggcctttttcaggtgtaacggcttgcctcgacttttgtgctcacgctcacagggatctgcacaacatgcagaacggcagcacactcgtttgtactctgaccagagaagacaatcgagagttcggtgggaaacctgaggacgaacaacttcacgtcttgccactgtacaaagtctctgatgttgatgaatttgggtcagttgaagcccaagaggaaaaaaaacgatcaggggcgatccaagtactgtcatcatttcggagaaaggtaagaatgcttgccgagcctgtcaaaacgtgccgccaacgcaaactggaggccaaaaaggcagcagcagaaaagttgagttctcttgagaacagctcaaacaaaaacgagaaagaaaagagcgcaccgtcacggacaaagcagacagaaaacgcttcacaagcgaagcagctcgcagaactccttcggttgtcaggccccgtcatgcaacaaagtcaacagccccagccgctgcagaaacagccaccccagccccaacagcaacagagaccgcaacaacaacagccccatcacccgcagacagaaagcgtaaatagctattccgcgtcagggtccaccaacccttatatgcggaggcccaatcctgtgagtccatatcccaattccagtcacacttccgatatctatgggagcacaagccccatgaatttttactctacttcatctcaggccgcaggttcatatcttaatagctccaatccgatgaatccctatcccggcctccttaatcagaatacccagtacccatcataccaatgcaacggaaacctgtcagttgataactgctccccgtaccttggaagctactccccacagtcccagcccatggatctgtaccggtatccaagtcaggacccactgtctaaattgagtctgccgccaattcacactctttatcaaccgagatttggaaacagccagtcattcacatctaaatacctgggttatggaaatcagaatatgcaaggagatggattctcctcatgtaccattcgaccaaacgttcaccatgttggcaagctcccaccttaccccacccatgaaatggatggtcatttcatgggagcgacctctcgattgccgccaaatctgagtaatcctaacatggattataaaaatggtgaacaccatagcccttcccatattatccataactattctgcagccccagggatgtttaactcttcattgcacgctttgcatttgcagaacaaggagaatgatatgctgtcccataccgcaaacggactcagtaaaatgcttccagcgcttaatcatgaccggacagcgtgcgtccaaggtgggcttcacaagctttccgacgccaacggtcaggagaagcagcctctggctttggtccagggggtggcgtccggagcagaagacaacgacgaagtatggagcgatagcgagcagagcttcctggatcctgacattgggggcgtggccgtggcaccgacccatggctctatccttatagagtgcgcgaaacgagagctccacgccaccacaccgcttaagaatcccaatcgcaatcatccaacacgcatcagtcttgtattttatcaacacaagtccatgaatgagcccaagcacggtctggccctttgggaagctaaaatggctgaaaaagcgagggagaaggaagaagaatgcgaaaagtatgggcccgattacgttcctcaaaaaagtcatggaaaaaaagtcaagcgcgagccagctgaacctcacgagacttcagaacctacgtacttgagattcattaagtctcttgctgaacggaccatgtctgtaaccacagactcaacggttaccacctctccatatgccttcacgagagttactggcccatataaccggtatata

SEQ ID NO: 48 - protein, 888 aa, synthetic construct
  REGION 1..888: note = Synthetic
MGGLDYKDDDDKASDFPSCRCVEQIIEKDEGPFYTHLGAGPNVAAIREIMEERFGQKGKAIRIERVIYTGKEGKSSQGCPIAKWVVRRSSSEEKLLCLVRERAGHTCEAAVIVILILVWEGIPLSLADKLYSELTETLRKYGTLTNRRCALNEERTCACQGLDPETCGASFSFGCSWSMYYNGCKFARSKIPRKFKLLGDDPKEEEKLESHLQNLSTLMAPTYKKLAPDAYNNQIEYEHRAPECRLGLKEGRPFSGVTACLDFCAHAHRDLHNMQNGSTLVCTLTREDNREFGGKPEDEQLHVLPLYKVSDVDEFGSVEAQEEKKRSGAIQVLSSFRRKVRMLAEPVKTCRQRKLEAKKAAAEKLSSLENSSNKNEKEKSAPSRTKQTENASQAKQLAELLRLSGPVMQQSQQPQPLQKQPPQPQQQQRPQQQQPHHPQTESVNSYSASGSTNPYMRRPNPVSPYPNSSHTSDIYGSTSPMNFYSTSSQAAGSYLNSSNPMNPYPGLLNQNTQYPSYQCNGNLSVDNCSPYLGSYSPQSQPMDLYRYPSQDPLSKLSLPPIHTLYQPRFGNSQSFTSKYLGYGNQNMQGDGFSSCTIRPNVHHVGKLPPYPTHEMDGHFMGATSRLPPNLSNPNMDYKNGEHHSPSHIIHNYSAAPGMFNSSLHALHLQNKENDMLSHTANGLSKMLPALNHDRTACVQGGLHKLSDANGQEKQPLALVQGVASGAEDNDEVWSDSEQSFLDPDIGGVAVAPTHGSILIECAKRELHATTPLKNPNRNHPTRISLVFYQHKSMNEPKHGLALWEAKMAEKAREKEEECEKYGPDYVPQKSHGKKVKREPAEPHETSEPTYLRFIKSLAERTMSVTTDSTVTTSPYAFTRVTGPYNRYI

SEQ ID NO: 49 - DNA, 939 bp, synthetic construct
  misc_feature 1..939: note = Synthetic
  source 1..939: mol_type = other DNA
aagggcggcggcggctccggcggcggcggctccaatcatgaccaagaatttgatccacctaaggtatatccccctgtgccagcagaaaaaagaaagcccattcgggtccttagcctcttcgacgggatagccaccgggctccttgtcctgaaagaccttggcatacaggtagatcggtatatcgcttccgaggtctgtgaagactcaatcaccgttgggatggtgcgccaccagggcaaaattatgtacgttggagatgtccgatcagtcactcagaaacatatccaagagtgggggccctttgacctcgtcatcggtgggtccccctgcaatgacttgtcaatagtcaacccggcgcggaagggtctctacgaaggtaccgggaggctcttttttgaattttataggctccttcatgatgctagaccgaaggaaggagatgatcgaccatttttctggctgtttgagaatgtggttgccatgggtgtgtcagataagcgggatatatcaagattcctcgagagcaacccggtaatgattgacgccaaagaagtaagcgcggcacaccgagctaggtacttctggggaaacctgccgggaatgaatagacccttggccagcacagtaaatgacaaactcgagttgcaagagtgtcttgagcacggtcgcatagctaaattcagtaaagtaaggacgatcaccactcggagtaatagtattaagcaagggaaggatcagcatttcccagtgttcatgaacgaaaaggaggacatcctttggtgcaccgaaatggagagagtattcgggttcccagttcattacacagatgtgagcaatatgtcacgactggctcggcaacggctcctggggagatcatggagtgtcccggttattagacatctctttgctcctctcaaagaatacttcgcttgtgtgtaa

SEQ ID NO: 50 - protein, 312 aa, synthetic construct
  REGION 1..312: note = Synthetic
KGGGGSGGGGSNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACV

SEQ ID NO: 51 - DNA, 843 bp, synthetic construct
  misc_feature 1..843: note = Synthetic
  source 1..843: mol_type = other DNA
atgggaagcgagaatcccttggaaatgtttgaaaccgtgcctgtttggagaagacaaccggtgcgcgtactttcattgttcgaagacattaaaaaggagctcaccagccttgggttcctcgagagtggaagcgatcctggtcagttgaaacacgtggttgacgtcacagacacggtgaggaaggatgttgaggagtggggtccattcgatttggtctatggcgcaacccctccgcttggacatacgtgcgatagacctccgagctggtatcttttccagtttcacaggctgcttcaatacgcaagaccgaagccgggttcccctcggcccttcttctggatgtttgtcgataatctcgttttgaataaagaggatttggatgtagcatcccgattccttgaaatggaaccggtaacgatccccgatgttcatggtgggagcttgcagaacgctgtccgggtgtggagtaacatacctgccatccggtcaaggcactgggctctcgtaagtgaggaagaactctcactgctggcgcagaacaagcagtcttctaaattggcggcaaaatggcccaccaagcttgtgaaaaattgctttctgccccttcgagagtattttaagtactttagtactgaactcacatcatcactgggcagtggtagtggctccggagctccacgaaacggcacggtccacctttacttgacgaaaccgctttatacctccgcaccgtccctccagcacctgtgtcggctgacaatcaataagtgtacaggtgcgatatggggattgccgcttccgacccgccttaaagattacctcgaggaatacaaatttcaagtc

SEQ ID NO: 52 - protein, 281 aa, synthetic construct
  REGION 1..281: note = Synthetic
MGSENPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSLGSGSGSGAPRNGTVHLYLTKPLYTSAPSLQHLCRLTINKCTGAIWGLPLPTRLKDYLEEYKFQV

SEQ ID NO: 53 - DNA, 1038 bp, synthetic construct
  misc_feature 1..1038: note = Synthetic
  source 1..1038: mol_type = other DNA
atgggaggccttgattacaaggacgacgacgacaaggctagcgagaatcccttggaaatgtttgaaaccgtgcctgtttggagaagacaaccggtgcgcgtactttcattgttcgaagacattaaaaaggagctcaccagccttgggttcctcgagagtggaagcgatcctggtcagttgaaacacgtggttgacgtcacagacacggtgaggaaggatgttgaggagtggggtccattcgatttggtctatggcgcaacccctccgcttggacatacgtgcgatagacctccgagctggtatcttttccagtttcacaggctgcttcaatacgcaagaccgaagccgggttcccctcggcccttcttctggatgtttgtcgataatctcgttttgaataaagaggatttggatgtagcatcccgattccttgaaatggaaccggtaacgatccccgatgttcatggtgggagcttgcagaacgctgtccgggtgtggagtaacatacctgccatccggtcaaggcactgggctctcgtaagtgaggaagaactctcactgctggcgcagaacaagcagtcttctaaattggcggcaaaatggcccaccaagcttgtgaaaaattgctttctgccccttcgagagtattttaagtactttagtactgaactcacatcatcactgggcagtggtagtggctccggagcagacccgagggaaagagttccaccaggtaacagtggcgaagaaacgatcggcgaagcgttcgagtggcttgatcggactattgaggctcttaatcgggaggctgtgaaccacctgccccgggaacttattttccaggtctggcagagaagttgggcctactggcacgatgagcaaggcatgtccacatcctatacaaagtatcggtatctttgtataatgcagaaggctgtatatatccactttaagaagggatgtacttgtctgggtagaggtcatggaccaggagggtggagaccaggccctccacctcctccacctccaggtcttgtg

SEQ ID NO: 54 - protein, 346 aa, synthetic construct
  REGION 1..346: note = Synthetic
MGGLDYKDDDDKASENPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSLGSGSGSGADPRERVPPGNSGEETIGEAFEWLDRTIEALNREAVNHLPRELIFQVWQRSWAYWHDEQGMSTSYTKYRYLCIMQKAVYIHFKKGCTCLGRGHGPGGWRPGPPPPPPPGLV

SEQ ID NO: 55 - DNA, 888 bp, synthetic construct
  misc_feature 1..888: note = Synthetic
  source 1..888: mol_type = other DNA
atgggaggccttgattacaaggacgacgacgacaaggctagcttcaatgtcttaatggttcatcggcgaagccatactggtgaacgcccattgcagtgcgaaatatgcggctttacctgccgccagcgcggtaacctcctccgccacattcgtctgcacacaggggaacggccttttcggtgtcacctctgcaactatgcatgccaaagaagagatgcgctcagcgggtcaggtagtggctccggagagaatcccttggaaatgtttgaaaccgtgcctgtttggagaagacaaccggtgcgcgtactttcattgttcgaagacattaaaaaggagctcaccagccttgggttcctcgagagtggaagcgatcctggtcagttgaaacacgtggttgacgtcacagacacggtgaggaaggatgttgaggagtggggtccattcgatttggtctatggcgcaacccctccgcttggacatacgtgcgatagacctccgagctggtatcttttccagtttcacaggctgcttcaatacgcaagaccgaagccgggttcccctcggcccttcttctggatgtttgtcgataatctcgttttgaataaagaggatttggatgtagcatcccgattccttgaaatggaaccggtaacgatccccgatgttcatggtgggagcttgcagaacgctgtccgggtgtggagtaacatacctgccatccggtcaaggcactgggctctcgtaagtgaggaagaactctcactgctggcgcagaacaagcagtcttctaaattggcggcaaaatggcccaccaagcttgtgaaaaattgctttctgccccttcgagagtattttaagtactttagtactgaactcacatcatcactg

SEQ ID NO: 56 - protein, 296 aa, synthetic construct
  REGION 1..296: note = Synthetic
MGGLDYKDDDDKASFNVLMVHRRSHTGERPLQCEICGFTCRQRGNLLRHIRLHTGERPFRCHLCNYACQRRDALSGSGSGSGENPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSL

SEQ ID NO: 57 - DNA, 843 bp, synthetic construct
  misc_feature 1..843: note = Synthetic
  source 1..843: mol_type = other DNA
atgggaagcgagaatcccttggaaatgtttgaaaccgtgcctgtttggagaagacaaccggtgcgcgtactttcattgttcgaagacattagaagggagctcaccagccttgggttcctcgagagtggaagcgatcctggtcagttgagacacgtggttgacgtcacagacacggtgaggcgggatgttgaggagtggggtccattcgatttggtctatggcgcaacccctccgcttggacatacgtgcgatagacctccgagctggtatcttttccagtttcacaggctgcttcaatacgcaagaccgaggccgggttcccctcggcccttcttctggatgtttgtcgataatctcgttttgaatagagaggatttggatgtagcatcccgattccttgaaatggaaccggtaacgatccccgatgttcatggtgggagcttgcagaacgctgtccgggtgtggagtaacatacctgccatccggtcaaggcactgggctctcgtaagtgaggaagaactctcactgctggcgcagaaccggcagtcttctaggttggcggcaagatggcccacccggcttgtgagaaattgctttctgccccttcgagagtattttaggtactttagtactgaactcacatcatcactgggcagtggtagtggctccggagctccacgaaacggcacggtccacctttacttgacgaaaccgctttatacctccgcaccgtccctccagcacctgtgtcggctgacaatcaataagtgtacaggtgcgatatggggattgccgcttccgacccgccttaaagattacctcgaggaatacaaatttcaagtc

SEQ ID NO: 58 - protein, 281 aa, synthetic construct
  REGION 1..281: note = Synthetic
MGSENPLEMFETVPVWRRQPVRVLSLFEDIRRELTSLGFLESGSDPGQLRHVVDVTDTVRRDVEEWGPFDLVYGATPPLGHTCDRPPSWYLFQFHRLLQYARPRPGSPRPFFWMFVDNLVLNREDLDVASRFLEMEPVTIPDVHGGSLQNAVRVWSNIPAIRSRHWALVSEEELSLLAQNRQSSRLAARWPTRLVRNCFLPLREYFRYFSTELTSSLGSGSGSGAPRNGTVHLYLTKPLYTSAPSLQHLCRLTINKCTGAIWGLPLPTRLKDYLEEYKFQV

SEQ ID NO: 59 - DNA, 1005 bp, synthetic construct
  misc_feature 1..1005: note = Synthetic
  source 1..1005: mol_type = other DNA
atgggaagcgagaatcccttggaaatgtttgaaaccgtgcctgtttggagaagacaaccggtgcgcgtactttcattgttcgaagacattagaagggagctcaccagccttgggttcctcgagagtggaagcgatcctggtcagttgagacacgtggttgacgtcacagacacggtgaggcgggatgttgaggagtggggtccattcgatttggtctatggcgcaacccctccgcttggacatacgtgcgatagacctccgagctggtatcttttccagtttcacaggctgcttcaatacgcaagaccgaggccgggttcccctcggcccttcttctggatgtttgtcgataatctcgttttgaatagagaggatttggatgtagcatcccgattccttgaaatggaaccggtaacgatccccgatgttcatggtgggagcttgcagaacgctgtccgggtgtggagtaacatacctgccatccggtcaaggcactgggctctcgtaagtgaggaagaactctcactgctggcgcagaaccggcagtcttctaggttggcggcaagatggcccacccggcttgtgagaaattgctttctgccccttcgagagtattttaggtactttagtactgaactcacatcatcactgggcagtggtagtggctccggagcagacccgagggaaagagttccaccaggtaacagtggcgaagaaacgatcggcgaagcgttcgagtggcttgatcggactattgaggctcttaatcgggaggctgtgaaccacctgccccgggaacttattttccaggtctggcagagaagttgggcctactggcacgatgagcaaggcatgtccacatcctatacaaagtatcggtatctttgtataatgcagaaggctgtatatatccactttaagaagggatgtacttgtctgggtagaggtcatggaccaggagggtggagaccaggccctccacctcctccacctccaggtcttgtg

SEQ ID NO: 60 - protein, 335 aa, synthetic construct
  REGION 1..335: note = Synthetic
MGSENPLEMFETVPVWRRQPVRVLSLFEDIRRELTSLGFLESGSDPGQLRHVVDVTDTVRRDVEEWGPFDLVYGATPPLGHTCDRPPSWYLFQFHRLLQYARPRPGSPRPFFWMFVDNLVLNREDLDVASRFLEMEPVTIPDVHGGSLQNAVRVWSNIPAIRSRHWALVSEEELSLLAQNRQSSRLAARWPTRLVRNCFLPLREYFRYFSTELTSSLGSGSGSGADPRERVPPGNSGEETIGEAFEWLDRTIEALNREAVNHLPRELIFQVWQRSWAYWHDEQGMSTSYTKYRYLCIMQKAVYIHFKKGCTCLGRGHGPGGWRPGPPPPPPPGLV

SEQ ID NO: 61 - DNA, 855 bp, synthetic construct
  misc_feature 1..855: note = Synthetic
  source 1..855: mol_type = other DNA
atgggaagcttcaatgtcttaatggttcatcggcgaagccatactggtgaacgcccattgcagtgcgaaatatgcggctttacctgccgccagcgcggtaacctcctccgccacattcgtctgcacacaggggaacggccttttcggtgtcacctctgcaactatgcatgccaaagaagagatgcgctcagcgggtcaggtagtggctccggagagaatcccttggaaatgtttgaaaccgtgcctgtttggagaagacaaccggtgcgcgtactttcattgttcgaagacattagaagggagctcaccagccttgggttcctcgagagtggaagcgatcctggtcagttgagacacgtggttgacgtcacagacacggtgaggcgggatgttgaggagtggggtccattcgatttggtctatggcgcaacccctccgcttggacatacgtgcgatagacctccgagctggtatcttttccagtttcacaggctgcttcaatacgcaagaccgaggccgggttcccctcggcccttcttctggatgtttgtcgataatctcgttttgaatagagaggatttggatgtagcatcccgattccttgaaatggaaccggtaacgatccccgatgttcatggtgggagcttgcagaacgctgtccgggtgtggagtaacatacctgccatccggtcaaggcactgggctctcgtaagtgaggaagaactctcactgctggcgcagaaccggcagtcttctaggttggcggcaagatggcccacccggcttgtgagaaattgctttctgccccttcgagagtattttaggtactttagtactgaactcacatcatcactg

SEQ ID NO: 62 - protein, 285 aa, synthetic construct
  REGION 1..285: note = Synthetic
MGSFNVLMVHRRSHTGERPLQCEICGFTCRQRGNLLRHIRLHTGERPFRCHLCNYACQRRDALSGSGSGSGENPLEMFETVPVWRRQPVRVLSLFEDIRRELTSLGFLESGSDPGQLRHVVDVTDTVRRDVEEWGPFDLVYGATPPLGHTCDRPPSWYLFQFHRLLQYARPRPGSPRPFFWMFVDNLVLNREDLDVASRFLEMEPVTIPDVHGGSLQNAVRVWSNIPAIRSRHWALVSEEELSLLAQNRQSSRLAARWPTRLVRNCFLPLREYFRYFSTELTSSL

SEQ ID NO: 63 - DNA, 828 bp, synthetic construct
  misc_feature 1..828: note = Synthetic
  source 1..828: mol_type = other DNA
atgggaggccttgattacaaggacgacgacgacaaggctagcatgcaccaccggaacgattcacaaaggctgggtaaggcgggctgccctccggagccaagcctccagatggcaaatacgaattttcttagtacgctgtcaccggaacattgtagaccgctggcgggagagtgcatgaacaaacttaaatgcggagctgctgaagccgagattatgaacctcccggaacgggtcggcactttcagtgctattccagcgctggggggaataagcctccctccgggagtaatcgtaatgactgcgcttcattcacccgctgcagcaagcgccgcggtgaccgatagtgcgtttcagattgcaaaccttgctgactgcccccagaatcacagttcctcaagttccagttctagtggtggagccggcggggctaaccccgccaagaaaaaacggaagagatgcggtgtttgtgttccatgtaaaagactgattaactgtggcgtgtgtagttcctgtcgcaataggaaagctggagcggctatatgtaaatttcggaagtgcgaggaattgaaaaaaaagcccggaaccagcctggagcggacgccggtcccatctgcagaagcctttaggtggttcttcggcagtggtagtggctccggagctccacgaaacggcacggtccacctttacttgacgaaaccgctttatacctccgcaccgtccctccagcacctgtgtcggctgacaatcaataagtgtacaggtgcgatatggggattgccgcttccgacccgccttaaagattacctcgaggaatacaaatttcaagtc

SEQ ID NO: 64 - protein, 276 aa, synthetic construct
  REGION 1..276: note = Synthetic
MGGLDYKDDDDKASMHHRNDSQRLGKAGCPPEPSLQMANTNFLSTLSPEHCRPLAGECMNKLKCGAAEAEIMNLPERVGTFSAIPALGGISLPPGVIVMTALHSPAAASAAVTDSAFQIANLADCPQNHSSSSSSSSGGAGGANPAKKKRKRCGVCVPCKRLINCGVCSSCRNRKAGAAICKFRKCEELKKKPGTSLERTPVPSAEAFRWFFGSGSGSGAPRNGTVHLYLTKPLYTSAPSLQHLCRLTINKCTGAIWGLPLPTRLKDYLEEYKFQV

SEQ ID NO: 65 - DNA, 990 bp, synthetic construct
  misc_feature 1..990: note = Synthetic
  source 1..990: mol_type = other DNA
atgggaggccttgattacaaggacgacgacgacaaggctagcatgcaccaccggaacgattcacaaaggctgggtaaggcgggctgccctccggagccaagcctccagatggcaaatacgaattttcttagtacgctgtcaccggaacattgtagaccgctggcgggagagtgcatgaacaaacttaaatgcggagctgctgaagccgagattatgaacctcccggaacgggtcggcactttcagtgctattccagcgctggggggaataagcctccctccgggagtaatcgtaatgactgcgcttcattcacccgctgcagcaagcgccgcggtgaccgatagtgcgtttcagattgcaaaccttgctgactgcccccagaatcacagttcctcaagttccagttctagtggtggagccggcggggctaaccccgccaagaaaaaacggaagagatgcggtgtttgtgttccatgtaaaagactgattaactgtggcgtgtgtagttcctgtcgcaataggaaagctggagcggctatatgtaaatttcggaagtgcgaggaattgaaaaaaaagcccggaaccagcctggagcggacgccggtcccatctgcagaagcctttaggtggttcttcggcagtggtagtggctccggagcagacccgagggaaagagttccaccaggtaacagtggcgaagaaacgatcggcgaagcgttcgagtggcttgatcggactattgaggctcttaatcgggaggctgtgaaccacctgccccgggaacttattttccaggtctggcagagaagttgggcctactggcacgatgagcaaggcatgtccacatcctatacaaagtatcggtatctttgtataatgcagaaggctgtatatatccactttaagaagggatgtacttgtctgggtagaggtcatggaccaggagggtggagaccaggccctccacctcctccacctccaggtcttgtg

SEQ ID NO: 66 - protein, 330 aa, synthetic construct
  REGION 1..330: note = Synthetic
MGGLDYKDDDDKASMHHRNDSQRLGKAGCPPEPSLQMANTNFLSTLSPEHCRPLAGECMNKLKCGAAEAEIMNLPERVGTFSAIPALGGISLPPGVIVMTALHSPAAASAAVTDSAFQIANLADCPQNHSSSSSSSSGGAGGANPAKKKRKRCGVCVPCKRLINCGVCSSCRNRKAGAAICKFRKCEELKKKPGTSLERTPVPSAEAFRWFFGSGSGSGADPRERVPPGNSGEETIGEAFEWLDRTIEALNREAVNHLPRELIFQVWQRSWAYWHDEQGMSTSYTKYRYLCIMQKAVYIHFKKGCTCLGRGHGPGGWRPGPPPPPPPGLV

SEQ ID NO: 67 - DNA, 840 bp, synthetic construct
  misc_feature 1..840: note = Synthetic
  source 1..840: mol_type = other DNA
atgggaggccttgattacaaggacgacgacgacaaggctagcttcaatgtcttaatggttcatcggcgaagccatactggtgaacgcccattgcagtgcgaaatatgcggctttacctgccgccagcgcggtaacctcctccgccacattcgtctgcacacaggggaacggccttttcggtgtcacctctgcaactatgcatgccaaagaagagatgcgctcagcgggtcaggtagtggctccggaatgcaccaccggaacgattcacaaaggctgggtaaggcgggctgccctccggagccaagcctccagatggcaaatacgaattttcttagtacgctgtcaccggaacattgtagaccgctggcgggagagtgcatgaacaaacttaaatgcggagctgctgaagccgagattatgaacctcccggaacgggtcggcactttcagtgctattccagcgctggggggaataagcctccctccgggagtaatcgtaatgactgcgcttcattcacccgctgcagcaagcgccgcggtgaccgatagtgcgtttcagattgcaaaccttgctgactgcccccagaatcacagttcctcaagttccagttctagtggtggagccggcggggctaaccccgccaagaaaaaacggaagagatgcggtgtttgtgttccatgtaaaagactgattaactgtggcgtgtgtagttcctgtcgcaataggaaagctggagcggctatatgtaaatttcggaagtgcgaggaattgaaaaaaaagcccggaaccagcctggagcggacgccggtcccatctgcagaagcctttaggtggttcttc

SEQ ID NO: 68 - protein, 280 aa, synthetic construct
  REGION 1..280: note = Synthetic
MGGLDYKDDDDKASFNVLMVHRRSHTGERPLQCEICGFTCRQRGNLLRHIRLHTGERPFRCHLCNYACQRRDALSGSGSGSGMHHRNDSQRLGKAGCPPEPSLQMANTNFLSTLSPEHCRPLAGECMNKLKCGAAEAEIMNLPERVGTFSAIPALGGISLPPGVIVMTALHSPAAASAAVTDSAFQIANLADCPQNHSSSSSSSSGGAGGANPAKKKRKRCGVCVPCKRLINCGVCSSCRNRKAGAAICKFRKCEELKKKPGTSLERTPVPSAEAFRWFF

SEQ ID NO: 69 - DNA, 795 bp, synthetic construct
  misc_feature 1..795: note = Synthetic
  source 1..795: mol_type = other DNA
atgggaagcatgcaccaccggaacgattcacaaaggctgggtcgggcgggctgccctccggagccaagcctccagatggcaaatacgaattttcttagtacgctgtcaccggaacattgtagaccgctggcgggagagtgcatgaacagacttaggtgcggagctgctgaagccgagattatgaacctcccggaacgggtcggcactttcagtgctattccagcgctggggggaataagcctccctccgggagtaatcgtaatgactgcgcttcattcacccgctgcagcaagcgccgcggtgaccgatagtgcgtttcagattgcaaaccttgctgactgcccccagaatcacagttcctcaagttccagttctagtggtggagccggcggggctaaccccgcccggagaagacggaggagatgcggtgtttgtgttccatgtagaagactgattaactgtggcgtgtgtagttcctgtcgcaataggagagctggagcggctatatgtaggtttcggaggtgcgaggaattgagaagacggcccggaaccagcctggagcggacgccggtcccatctgcagaagcctttaggtggttcttcggcagtggtagtggctccggagctccacgaaacggcacggtccacctttacttgacgaaaccgctttatacctccgcaccgtccctccagcacctgtgtcggctgacaatcaataagtgtacaggtgcgatatggggattgccgcttccgacccgccttaaagattacctcgaggaatacaaatttcaagtc

SEQ ID NO: 70 - protein, 265 aa, synthetic construct
  REGION 1..265: note = Synthetic
MGSMHHRNDSQRLGRAGCPPEPSLQMANTNFLSTLSPEHCRPLAGECMNRLRCGAAEAEIMNLPERVGTFSAIPALGGISLPPGVIVMTALHSPAAASAAVTDSAFQIANLADCPQNHSSSSSSSSGGAGGANPARRRRRRCGVCVPCRRLINCGVCSSCRNRRAGAAICRFRRCEELRRRPGTSLERTPVPSAEAFRWFFGSGSGSGAPRNGTVHLYLTKPLYTSAPSLQHLCRLTINKCTGAIWGLPLPTRLKDYLEEYKFQV

SEQ ID NO: 71 - DNA, 957 bp, synthetic construct
  misc_feature 1..957: note = Synthetic
  source 1..957: mol_type = other DNA
atgggaagcatgcaccaccggaacgattcacaaaggctgggtcgggcgggctgccctccggagccaagcctccagatggcaaatacgaattttcttagtacgctgtcaccggaacattgtagaccgctggcgggagagtgcatgaacagacttaggtgcggagctgctgaagccgagattatgaacctcccggaacgggtcggcactttcagtgctattccagcgctggggggaataagcctccctccgggagtaatcgtaatgactgcgcttcattcacccgctgcagcaagcgccgcggtgaccgatagtgcgtttcagattgcaaaccttgctgactgcccccagaatcacagttcctcaagttccagttctagtggtggagccggcggggctaaccccgcccggagaagacggaggagatgcggtgtttgtgttccatgtagaagactgattaactgtggcgtgtgtagttcctgtcgcaataggagagctggagcggctatatgtaggtttcggaggtgcgaggaattgagaagacggcccggaaccagcctggagcggacgccggtcccatctgcagaagcctttaggtggttcttcggcagtggtagtggctccggagcagacccgagggaaagagttccaccaggtaacagtggcgaagaaacgatcggcgaagcgttcgagtggcttgatcggactattgaggctcttaatcgggaggctgtgaaccacctgccccgggaacttattttccaggtctggcagagaagttgggcctactggcacgatgagcaaggcatgtccacatcctatacaaagtatcggtatctttgtataatgcagaaggctgtatatatccactttaagaagggatgtacttgtctgggtagaggtcatggaccaggagggtggagaccaggccctccacctcctccacctccaggtcttgtg

SEQ ID NO: 72 - protein, 319 aa, synthetic construct
  REGION 1..319: note = Synthetic
MGSMHHRNDSQRLGRAGCPPEPSLQMANTNFLSTLSPEHCRPLAGECMNRLRCGAAEAEIMNLPERVGTFSAIPALGGISLPPGVIVMTALHSPAAASAAVTDSAFQIANLADCPQNHSSSSSSSSGGAGGANPARRRRRRCGVCVPCRRLINCGVCSSCRNRRAGAAICRFRRCEELRRRPGTSLERTPVPSAEAFRWFFGSGSGSGADPRERVPPGNSGEETIGEAFEWLDRTIEALNREAVNHLPRELIFQVWQRSWAYWHDEQGMSTSYTKYRYLCIMQKAVYIHFKKGCTCLGRGHGPGGWRPGPPPPPPPGLV

SEQ ID NO: 73 - DNA, 807 bp, synthetic construct
  misc_feature 1..807: note = Synthetic
  source 1..807: mol_type = other DNA
atgggaagcttcaatgtcttaatggttcatcggcgaagccatactggtgaacgcccattgcagtgcgaaatatgcggctttacctgccgccagcgcggtaacctcctccgccacattcgtctgcacacaggggaacggccttttcggtgtcacctctgcaactatgcatgccaaagaagagatgcgctcagcgggtcaggtagtggctccggaatgcaccaccggaacgattcacaaaggctgggtcgggcgggctgccctccggagccaagcctccagatggcaaatacgaattttcttagtacgctgtcaccggaacattgtagaccgctggcgggagagtgcatgaacagacttaggtgcggagctgctgaagccgagattatgaacctcccggaacgggtcggcactttcagtgctattccagcgctggggggaataagcctccctccgggagtaatcgtaatgactgcgcttcattcacccgctgcagcaagcgccgcggtgaccgatagtgcgtttcagattgcaaaccttgctgactgcccccagaatcacagttcctcaagttccagttctagtggtggagccggcggggctaaccccgcccggagaagacggaggagatgcggtgtttgtgttccatgtagaagactgattaactgtggcgtgtgtagttcctgtcgcaataggagagctggagcggctatatgtaggtttcggaggtgcgaggaattgagaagacggcccggaaccagcctggagcggacgccggtcccatctgcagaagcctttaggtggttcttc

SEQ ID NO: 74 - protein, 269 aa, synthetic construct
  REGION 1..269: note = Synthetic
MGSFNVLMVHRRSHTGERPLQCEICGFTCRQRGNLLRHIRLHTGERPFRCHLCNYACQRRDALSGSGSGSGMHHRNDSQRLGRAGCPPEPSLQMANTNFLSTLSPEHCRPLAGECMNRLRCGAAEAEIMNLPERVGTFSAIPALGGISLPPGVIVMTALHSPAAASAAVTDSAFQIANLADCPQNHSSSSSSSSGGAGGANPARRRRRRCGVCVPCRRLINCGVCSSCRNRRAGAAICRFRRCEELRRRPGTSLERTPVPSAEAFRWFF

SEQ ID NO: 75 - DNA, 594 bp, synthetic construct
  misc_feature 1..594: note = Synthetic
  source 1..594: mol_type = other DNA
atgcaccaccggaacgattcacaaaggctgggtaaggcgggctgccctccggagccaagcctccagatggcaaatacgaattttcttagtacgctgtcaccggaacattgtagaccgctggcgggagagtgcatgaacaaacttaaatgcggagctgctgaagccgagattatgaacctcccggaacgggtcggcactttcagtgctattccagcgctggggggaataagcctccctccgggagtaatcgtaatgactgcgcttcattcacccgctgcagcaagcgccgcggtgaccgatagtgcgtttcagattgcaaaccttgctgactgcccccagaatcacagttcctcaagttccagttctagtggtggagccggcggggctaaccccgccaagaaaaaacggaagagatgcggtgtttgtgttccatgtaaaagactgattaactgtggcgtgtgtagttcctgtcgcaataggaaaactgggcaccagatatgtaaatttcggaagtgcgaggaattgaaaaaaaagcccggaaccagcctggagcggacgccggtcccatctgcagaagcctttaggtggttcttc

SEQ ID NO: 76 - protein, 198 aa, synthetic construct
  REGION 1..198: note = Synthetic
MHHRNDSQRLGKAGCPPEPSLQMANTNFLSTLSPEHCRPLAGECMNKLKCGAAEAEIMNLPERVGTFSAIPALGGISLPPGVIVMTALHSPAAASAAVTDSAFQIANLADCPQNHSSSSSSSSGGAGGANPAKKKRKRCGVCVPCKRLINCGVCSSCRNRKTGHQICKFRKCEELKKKPGTSLERTPVPSAEAFRWFF

SEQ ID NO: 77 - DNA, 288 bp, synthetic construct
  misc_feature 1..288: note = Synthetic
  source 1..288: mol_type = other DNA
atggagcaagcaccggaagatcagggaccacagcgcgaaccacacaatgaatggacattggaattgcttgaggagcttaaaaatgaagctgttagacacttcccaagaatctggctgcacggcttgggtcagcacatatatgaaacgtatggagatacatgggcaggtgtcgaggctatcatcaggatccttcagcagctcctcttcattcatttcagaatagggtgtcgacatagcagaataggggttacccagcaacgcagggcgcggaatggtgccagtaggagt

SEQ ID NO: 78 - protein, 96 aa, synthetic construct
  REGION 1..96: note = Synthetic
MEQAPEDQGPQREPHNEWTLELLEELKNEAVRHFPRIWLHGLGQHIYETYGDTWAGVEAIIRILQQLLFIHFRIGCRHSRIGVTQQRRARNGASRS

SEQ ID NO: 79 - DNA, 1053 bp, synthetic construct
  misc_feature 1..1053: note = Synthetic
  source 1..1053: mol_type = other DNA
atgtctgggttcaatgtgctgatggtccacaagagaagtcacaccggcgagcggcccctgcagtgcgaaatctgtggatttacatgccgccaaaaagggaacttgcttcgacacataaagctgcataccggcgagaagccatttaagtgccacttgtgtaactacgcttgtcagaggcgggacgcattggggagcggatctggatcaggaagcgggggagagaatcccttggaaatgtttgaaaccgtgcctgtttggagaagacaaccggtgcgcgtactttcattgttcgaagacattaaaaaggagctcaccagccttgggttcctcgagagtggaagcgatcctggtcagttgaaacacgtggttgacgtcacagacacggtgaggaaggatgttgaggagtggggtccattcgatttggtctatggcgcaacccctccgcttggacatacgtgcgatagacctccgagctggtatcttttccagtttcacaggctgcttcaatacgcaagaccgaagccgggttcccctcggcccttcttctggatgtttgtcgataatctcgttttgaataaagaggatttggatgtagcatcccgattccttgaaatggaaccggtaacgatccccgatgttcatggtgggagcttgcagaacgctgtccgggtgtggagtaacatacctgccatccggtcaaggcactgggctctcgtaagtgaggaagaactctcactgctggcgcagaacaagcagtcttctaaattggcggcaaaatggcccaccaagcttgtgaaaaattgctttctgccccttcgagagtattttaagtactttagtactgaactcacatcatcactgggcagtggtagtggctccggagctccacgaaacggcacggtccacctttacttgacgaaaccgctttatacctccgcaccgtccctccagcacctgtgtcggctgacaatcaataagtgtacaggtgcgatatggggattgccgcttccgacccgccttaaagattacctcgaggaatacaaatttcaagtc

SEQ ID NO: 80 - protein, 351 aa, synthetic construct
  REGION 1..351: note = Synthetic
MSGFNVLMVHKRSHTGERPLQCEICGFTCRQKGNLLRHIKLHTGEKPFKCHLCNYACQRRDALGSGSGSGSGGENPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSLGSGSGSGAPRNGTVHLYLTKPLYTSAPSLQHLCRLTINKCTGAIWGLPLPTRLKDYLEEYKFQV

SEQ ID NO: 81 - DNA, 1053 bp, synthetic construct
  misc_feature 1..1053: note = Synthetic
  source 1..1053: mol_type = other DNA
atgggaagcgagaatcccttggaaatgtttgaaaccgtgcctgtttggagaagacaaccggtgcgcgtactttcattgttcgaagacattaaaaaggagctcaccagccttgggttcctcgagagtggaagcgatcctggtcagttgaaacacgtggttgacgtcacagacacggtgaggaaggatgttgaggagtggggtccattcgatttggtctatggcgcaacccctccgcttggacatacgtgcgatagacctccgagctggtatcttttccagtttcacaggctgcttcaatacgcaagaccgaagccgggttcccctcggcccttcttctggatgtttgtcgataatctcgttttgaataaagaggatttggatgtagcatcccgattccttgaaatggaaccggtaacgatccccgatgttcatggtgggagcttgcagaacgctgtccgggtgtggagtaacatacctgccatccggtcaaggcactgggctctcgtaagtgaggaagaactctcactgctggcgcagaacaagcagtcttctaaattggcggcaaaatggcccaccaagcttgtgaaaaattgctttctgccccttcgagagtattttaagtactttagtactgaactcacatcatcactgggcagtggctcgggctcggggtccggcggattcaatgtcttaatggttcataagcgaagccatactggtgaacgcccattgcagtgcgaaatatgcggctttacctgccgccagaaaggtaacctcctccgccacattaaactgcacacaggggaaaaaccttttaagtgtcacctctgcaactatgcatgccaaagaagagatgcgctcggcagtggtagtggctccggagctccacgaaacggcacggtccacctttacttgacgaaaccgctttatacctccgcaccgtccctccagcacctgtgtcggctgacaatcaataagtgtacaggtgcgatatggggattgccgcttccgacccgccttaaagattacctcgaggaatacaaatttcaagtc

SEQ ID NO: 82 - protein, 351 aa, synthetic construct
  REGION 1..351: note = Synthetic
MGSENPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSLGSGSGSGSGGFNVLMVHKRSHTGERPLQCEICGFTCRQKGNLLRHIKLHTGEKPFKCHLCNYACQRRDALGSGSGSGAPRNGTVHLYLTKPLYTSAPSLQHLCRLTINKCTGAIWGLPLPTRLKDYLEEYKFQV

SEQ ID NO: 83 - DNA, 1053 bp, synthetic construct
  misc_feature 1..1053: note = Synthetic
  source 1..1053: mol_type = other DNA
atgggaagcgagaatcccttggaaatgtttgaaaccgtgcctgtttggagaagacaaccggtgcgcgtactttcattgttcgaagacattaaaaaggagctcaccagccttgggttcctcgagagtggaagcgatcctggtcagttgaaacacgtggttgacgtcacagacacggtgaggaaggatgttgaggagtggggtccattcgatttggtctatggcgcaacccctccgcttggacatacgtgcgatagacctccgagctggtatcttttccagtttcacaggctgcttcaatacgcaagaccgaagccgggttcccctcggcccttcttctggatgtttgtcgataatctcgttttgaataaagaggatttggatgtagcatcccgattccttgaaatggaaccggtaacgatccccgatgttcatggtgggagcttgcagaacgctgtccgggtgtggagtaacatacctgccatccggtcaaggcactgggctctcgtaagtgaggaagaactctcactgctggcgcagaacaagcagtcttctaaattggcggcaaaatggcccaccaagcttgtgaaaaattgctttctgccccttcgagagtattttaagtactttagtactgaactcacatcatcactgggcagtggtagtggctccggagctccacgaaacggcacggtccacctttacttgacgaaaccgctttatacctccgcaccgtccctccagcacctgtgtcggctgacaatcaataagtgtacaggtgcgatatggggattgccgcttccgacccgccttaaagattacctcgaggaatacaaatttcaagtcggcagtggctcgggctcggggtccggcggattcaatgtcttaatggttcataagcgaagccatactggtgaacgcccattgcagtgcgaaatatgcggctttacctgccgccagaaaggtaacctcctccgccacattaaactgcacacaggggaaaaaccttttaagtgtcacctctgcaactatgcatgccaaagaagagatgcgctc

SEQ ID NO: 84 - protein, 351 aa, synthetic construct
  REGION 1..351: note = Synthetic
MGSENPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSLGSGSGSGAPRNGTVHLYLTKPLYTSAPSLQHLCRLTINKCTGAIWGLPLPTRLKDYLEEYKFQVGSGSGSGSGGFNVLMVHKRSHTGERPLQCEICGFTCRQKGNLLRHIKLHTGEKPFKCHLCNYACQRRDAL

SEQ ID NO: 85 - DNA, 759 bp, synthetic construct
  misc_feature 1..759: note = Synthetic
  source 1..759: mol_type = other DNA
atgtctgggttcaatgtgctgatggtccacaagagaagtcacaccggcgagcggcccctgcagtgcgaaatctgtggatttacatgccgccaaaaagggaacttgcttcgacacataaagctgcataccggcgagaagccatttaagtgccacttgtgtaactacgcttgtcagaggcgggacgcattggggagcggatctggatcaggaagcgggggagatcaagtccaactggtggagtctggtggcgctttggtgcagccaggtggctctctgcgtttgtcctgtgccgcttctggcttcccagtgaaccgctattccatgcgctggtatcgccaggctccaggcaaagagcgtgagtgggtagccggtatgtccagcgcgggtgatcgtagctcctatgaagactccgtgaagggccgtttcaccatcagccgtgacgatgcccgtaacacggtgtatctgcaaatgaacagcttgaaacctgaagatacggccgtgtattactgtaatgtgaacgtgggcttcgagtattggggccaaggcacccaggtcaccgtgtccagcggcagtggtagtggctccggagctccacgaaacggcacggtccacctttacttgacgaaaccgctttatacctccgcaccgtccctccagcacctgtgtcggctgacaatcaataagtgtacaggtgcgatatggggattgccgcttccgacccgccttaaagattacctcgaggaatacaaatttcaagtc

SEQ ID NO: 86 - protein, 253 aa, synthetic construct
  REGION 1..253: note = Synthetic
MSGFNVLMVHKRSHTGERPLQCEICGFTCRQKGNLLRHIKLHTGEKPFKCHLCNYACQRRDALGSGSGSGSGGDQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGSGSGSGAPRNGTVHLYLTKPLYTSAPSLQHLCRLTINKCTGAIWGLPLPTRLKDYLEEYKFQV

SEQ ID NO: 87 - DNA, 792 bp, synthetic construct
  misc_feature 1..792: note = Synthetic
  source 1..792: mol_type = other DNA
atgggaggccttgattacaaggacgacgacgacaaggctagcgatcaagtccaactggtggagtctggtggcgctttggtgcagccaggtggctctctgcgtttgtcctgtgccgcttctggcttcccagtgaaccgctattccatgcgctggtatcgccaggctccaggcaaagagcgtgagtgggtagccggtatgtccagcgcgggtgatcgtagctcctatgaagactccgtgaagggccgtttcaccatcagccgtgacgatgcccgtaacacggtgtatctgcaaatgaacagcttgaaacctgaagatacggccgtgtattactgtaatgtgaacgtgggcttcgagtattggggccaaggcacccaggtcaccgtgtccagcggcagtggctcgggctcggggtccggcggattcaatgtcttaatggttcataagcgaagccatactggtgaacgcccattgcagtgcgaaatatgcggctttacctgccgccagaaaggtaacctcctccgccacattaaactgcacacaggggaaaaaccttttaagtgtcacctctgcaactatgcatgccaaagaagagatgcgctcggcagtggtagtggctccggagctccacgaaacggcacggtccacctttacttgacgaaaccgctttatacctccgcaccgtccctccagcacctgtgtcggctgacaatcaataagtgtacaggtgcgatatggggattgccgcttccgacccgccttaaagattacctcgaggaatacaaatttcaagtc

SEQ ID NO: 88 - protein, 264 aa, synthetic construct
  REGION 1..264: note = Synthetic
MGGLDYKDDDDKASDQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGSGSGSGSGGFNVLMVHKRSHTGERPLQCEICGFTCRQKGNLLRHIKLHTGEKPFKCHLCNYACQRRDALGSGSGSGAPRNGTVHLYLTKPLYTSAPSLQHLCRLTINKCTGAIWGLPLPTRLKDYLEEYKFQV

SEQ ID NO: 89 - DNA, 792 bp, synthetic construct
  misc_feature 1..792: note = Synthetic
  source 1..792: mol_type = other DNA
atgggaggccttgattacaaggacgacgacgacaaggctagcgatcaagtccaactggtggagtctggtggcgctttggtgcagccaggtggctctctgcgtttgtcctgtgccgcttctggcttcccagtgaaccgctattccatgcgctggtatcgccaggctccaggcaaagagcgtgagtgggtagccggtatgtccagcgcgggtgatcgtagctcctatgaagactccgtgaagggccgtttcaccatcagccgtgacgatgcccgtaacacggtgtatctgcaaatgaacagcttgaaacctgaagatacggccgtgtattactgtaatgtgaacgtgggcttcgagtattggggccaaggcacccaggtcaccgtgtccagcggcagtggtagtggctccggagctccacgaaacggcacggtccacctttacttgacgaaaccgctttatacctccgcaccgtccctccagcacctgtgtcggctgacaatcaataagtgtacaggtgcgatatggggattgccgcttccgacccgccttaaagattacctcgaggaatacaaatttcaagtcggcagtggctcgggctcggggtccggcggattcaatgtcttaatggttcataagcgaagccatactggtgaacgcccattgcagtgcgaaatatgcggctttacctgccgccagaaaggtaacctcctccgccacattaaactgcacacaggggaaaaaccttttaagtgtcacctctgcaactatgcatgccaaagaagagatgcgctc

SEQ ID NO: 90 - protein, 264 aa, synthetic construct
  REGION 1..264: note = Synthetic
MGGLDYKDDDDKASDQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGSGSGSGAPRNGTVHLYLTKPLYTSAPSLQHLCRLTINKCTGAIWGLPLPTRLKDYLEEYKFQVGSGSGSGSGGFNVLMVHKRSHTGERPLQCEICGFTCRQKGNLLRHIKLHTGEKPFKCHLCNYACQRRDAL

SEQ ID NO: 91 - DNA, 891 bp, synthetic construct
  misc_feature 1..891: note = Synthetic
  source 1..891: mol_type = other DNA
atggccccaaagaagaagcggaaggtcggtatccacggagtcccagcagccggaagcgagaatcccttggaaatgtttgaaaccgtgcctgtttggagaagacaaccggtgcgcgtactttcattgttcgaagacattaaaaaggagctcaccagccttgggttcctcgagagtggaagcgatcctggtcagttgaaacacgtggttgacgtcacagacacggtgaggaaggatgttgaggagtggggtccattcgatttggtctatggcgcaacccctccgcttggacatacgtgcgatagacctccgagctggtatcttttccagtttcacaggctgcttcaatacgcaagaccgaagccgggttcccctcggcccttcttctggatgtttgtcgataatctcgttttgaataaagaggatttggatgtagcatcccgattccttgaaatggaaccggtaacgatccccgatgttcatggtgggagcttgcagaacgctgtccgggtgtggagtaacatacctgccatccggtcaaggcactgggctctcgtaagtgaggaagaactctcactgctggcgcagaacaagcagtcttctaaattggcggcaaaatggcccaccaagcttgtgaaaaattgctttctgccccttcgagagtattttaagtactttagtactgaactcacatcatcactgggcagtggtagtggctccggagctccacgaaacggcacggtccacctttacttgacgaaaccgctttatacctccgcaccgtccctccagcacctgtgtcggctgacaatcaataagtgtacaggtgcgatatggggattgccgcttccgacccgccttaaagattacctcgaggaatacaaatttcaagtc

SEQ ID NO: 92 - protein, 297 aa, synthetic construct
  REGION 1..297: note = Synthetic
MAPKKKRKVGIHGVPAAGSENPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSLGSGSGSGAPRNGTVHLYLTKPLYTSAPSLQHLCRLTINKCTGAIWGLPLPTRLKDYLEEYKFQV

SEQ ID NO: 93 - DNA, 906 bp, synthetic construct
  misc_feature 1..906: note = Synthetic
  source 1..906: mol_type = other DNA
atgggaagcgagaatcccttggaaatgtttgaaaccgtgcctgtttggagaagacaaccggtgcgcgtactttcattgttcgaagacattaaaaaggagctcaccagccttgggttcctcgagagtggaagcgatcctggtcagttgaaacacgtggttgacgtcacagacacggtgaggaaggatgttgaggagtggggtccattcgatttggtctatggcgcaacccctccgcttggacatacgtgcgatagacctccgagctggtatcttttccagtttcacaggctgcttcaatacgcaagaccgaagccgggttcccctcggcccttcttctggatgtttgtcgataatctcgttttgaataaagaggatttggatgtagcatcccgattccttgaaatggaaccggtaacgatccccgatgttcatggtgggagcttgcagaacgctgtccgggtgtggagtaacatacctgccatccggtcaaggcactgggctctcgtaagtgaggaagaactctcactgctggcgcagaacaagcagtcttctaaattggcggcaaaatggcccaccaagcttgtgaaaaattgctttctgccccttcgagagtattttaagtactttagtactgaactcacatcatcactgggcagtggtagtggctccggagctccacgaaacggcacggtccacctttacttgacgaaaccgctttatacctccgcaccgtccctccagcacctgtgtcggctgacaatcaataagtgtacaggtgcgatatggggattgccgcttccgacccgccttaaagattacctcgaggaatacaaatttcaagtcggcggcggcggctccaagcgtcctgctgctactaagaaagctggtcaagctaagaaaaagaaa

SEQ ID NO: 94 - protein, 302 aa, synthetic construct
  REGION 1..302: note = Synthetic
MGSENPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSLGSGSGSGAPRNGTVHLYLTKPLYTSAPSLQHLCRLTINKCTGAIWGLPLPTRLKDYLEEYKFQVGGGGSKRPAATKKAGQAKKKK

SEQ ID NO: 95 - DNA, 870 bp, synthetic construct
  misc_feature 1..870: note = Synthetic
  source 1..870: mol_type = other DNA
aagggcggcggcggctccggcggcggcggctccgagaatcccttggaaatgtttgaaaccgtgcctgtttggagaagacaaccggtgcgcgtactttcattgttcgaagacattaaaaaggagctcaccagccttgggttcctcgagagtggaagcgatcctggtcagttgaaacacgtggttgacgtcacagacacggtgaggaaggatgttgaggagtggggtccattcgatttggtctatggcgcaacccctccgcttggacatacgtgcgatagacctccgagctggtatcttttccagtttcacaggctgcttcaatacgcaagaccgaagccgggttcccctcggcccttcttctggatgtttgtcgataatctcgttttgaataaagaggatttggatgtagcatcccgattccttgaaatggaaccggtaacgatccccgatgttcatggtgggagcttgcagaacgctgtccgggtgtggagtaacatacctgccatccggtcaaggcactgggctctcgtaagtgaggaagaactctcactgctggcgcagaacaagcagtcttctaaattggcggcaaaatggcccaccaagcttgtgaaaaattgctttctgccccttcgagagtattttaagtactttagtactgaactcacatcatcactgggcagtggtagtggctccggagctccacgaaacggcacggtccacctttacttgacgaaaccgctttatacctccgcaccgtccctccagcacctgtgtcggctgacaatcaataagtgtacaggtgcgatatggggattgccgcttccgacccgccttaaagattacctcgaggaatacaaatttcaagtctaa

SEQ ID NO: 96 - protein, 289 aa, synthetic construct
  REGION 1..289: note = Synthetic
KGGGGSGGGGSENPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSLGSGSGSGAPRNGTVHLYLTKPLYTSAPSLQHLCRLTINKCTGAIWGLPLPTRLKDYLEEYKFQV

SEQ ID NO: 97 - DNA, 585 bp, synthetic construct
  misc_feature 1..585: note = Synthetic
  source 1..585: mol_type = other DNA
atgggaggccttgattacaaggacgacgacgacaaggctagcgatcaagtccaactggtggagtctggtggcgctttggtgcagccaggtggctctctgcgtttgtcctgtgccgcttctggcttcccagtgaaccgctattccatgcgctggtatcgccaggctccaggcaaagagcgtgagtgggtagccggtatgtccagcgcgggtgatcgtagctcctatgaagactccgtgaagggccgtttcaccatcagccgtgacgatgcccgtaacacggtgtatctgcaaatgaacagcttgaaacctgaagatacggccgtgtattactgtaatgtgaacgtgggcttcgagtattggggccaaggcacccaggtcaccgtgtccagcggcagtggtagtggctccggagctccacgaaacggcacggtccacctttacttgacgaaaccgctttatacctccgcaccgtccctccagcacctgtgtcggctgacaatcaataagtgtacaggtgcgatatggggattgccgcttccgacccgccttaaagattacctcgaggaatacaaatttcaagtctaa

SEQ ID NO: 98 - protein, 194 aa, synthetic construct; identical to SEQ ID NO: 10
  REGION 1..194: note = Synthetic

SEQ ID NO: 99 - DNA, 1350 bp, synthetic construct
  misc_feature 1..1350: note = Synthetic
  source 1..1350: mol_type = other DNA
atgagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaatggttcaggtgcaacgaacttctcattgttgaagcaagccggtgatgttgaggaaaatccgggtcctatgggaggccttgattacaaggacgacgacgacaaggctagcgatcaagtccaactggtggagtctggtggcgctttggtgcagccaggtggctctctgcgtttgtcctgtgccgcttctggcttcccagtgaaccgctattccatgcgctggtatcgccaggctccaggcaaagagcgtgagtgggtagccggtatgtccagcgcgggtgatcgtagctcctatgaagactccgtgaagggccgtttcaccatcagccgtgacgatgcccgtaacacggtgtatctgcaaatgaacagcttgaaacctgaagatacggccgtgtattactgtaatgtgaacgtgggcttcgagtattggggccaaggcacccaggtcaccgtgtccagcggcagtggtagtggctccggagctccacgaaacggcacggtccacctttacttgacgaaaccgctttatacctccgcaccgtccctccagcacctgtgtcggctgacaatcaataagtgtacaggtgcgatatggggattgccgcttccgacccgccttaaagattacctcgaggaatacaaatttcaagtctaa

SEQ ID NO: 100 - protein, 449 aa, synthetic construct
  REGION 1..449: note = Synthetic
MSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLNGSGATNFSLLKQAGDVEENPGPMGGLDYKDDDDKASDQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGSGSGSGAPRNGTVHLYLTKPLYTSAPSLQHLCRLTINKCTGAIWGLPLPTRLKDYLEEYKFQV

SEQ ID NO: 101 - DNA, 363 bp, synthetic construct
  misc_feature 1..363: note = Synthetic
  source 1..363: mol_type = other DNA
atgagcgcgtcatcccagagccctaacccaaataatcccgcagaatactgcagcacaataccccctttgcagcaggcacaagctagtggcgctttgagcagcccaccgcccacggtcatggttcctgtgggagtccttaaacatccaggggctgaagtagcacagccccggggcagtggtagtggctccggagctccacgaaacggcacggtccacctttacttgacgaaaccgctttatacctccgcaccgtccctccagcacctgtgtcggctgacaatcaataagtgtacaggtgcgatatggggattgccgcttccgacccgccttaaagattacctcgaggaatacaaatttcaagtc

SEQ ID NO: 102 - protein, 121 aa, synthetic construct
  REGION 1..121: note = Synthetic
MSASSQSPNPNNPAEYCSTIPPLQQAQASGALSSPPPTVMVPVGVLKHPGAEVAQPRGSGSGSGAPRNGTVHLYLTKPLYTSAPSLQHLCRLTINKCTGAIWGLPLPTRLKDYLEEYKFQV

SEQ ID NO: 103 - DNA, 567 bp, synthetic construct
  misc_feature 1..567: note = Synthetic
  source 1..567: mol_type = other DNA
atgggaggccttgattacaaggacgacgacgacaaggctagcatgagcgcgtcatcccagagccctaacccaaataatcccgcagaatactgcagcacaataccccctttgcagcaggcacaagctagtggcgctttgagcagcccaccgcccacggtcatggttcctgtgggagtccttaaacatccaggggctgaagtagcacagccccggggcagtggtagtggctccggagcagacccgagggaaagagttccaccaggtaacagtggcgaagaaacgatcggcgaagcgttcgagtggcttgatcggactattgaggctcttaatcgggaggctgtgaaccacctgccccgggaacttattttccaggtctggcagagaagttgggcctactggcacgatgagcaaggcatgtccacatcctatacaaagtatcggtatctttgtataatgcagaaggctgtatatatccactttaagaagggatgtacttgtctgggtagaggtcatggaccaggagggtggagaccaggccctccacctcctccacctccaggtcttgtg

SEQ ID NO: 104 - protein, 189 aa, synthetic construct
  REGION 1..189: note = Synthetic
MGGLDYKDDDDKASMSASSQSPNPNNPAEYCSTIPPLQQAQASGALSSPPPTVMVPVGVLKHPGAEVAQPRGSGSGSGADPRERVPPGNSGEETIGEAFEWLDRTIEALNREAVNHLPRELIFQVWQRSWAYWHDEQGMSTSYTKYRYLCIMQKAVYIHFKKGCTCLGRGHGPGGWRPGPPPPPPPGLV

SEQ ID NO: 105 - DNA, 417 bp, synthetic construct
  misc_feature 1..417: note = Synthetic
  source 1..417: mol_type = other DNA
atgggaggccttgattacaaggacgacgacgacaaggctagcttcaatgtcttaatggttcatcggcgaagccatactggtgaacgcccattgcagtgcgaaatatgcggctttacctgccgccagcgcggtaacctcctccgccacattcgtctgcacacaggggaacggccttttcggtgtcacctctgcaactatgcatgccaaagaagagatgcgctcagcgggtcaggtagtggctccggaatgagcgcgtcatcccagagccctaacccaaataatcccgcagaatactgcagcacaataccccctttgcagcaggcacaagctagtggcgctttgagcagcccaccgcccacggtcatggttcctgtgggagtccttagacatccaggggctgaagtagcacagccccgg

SEQ ID NO: 106 - protein, 139 aa, synthetic construct
  REGION 1..139: note = Synthetic
MGGLDYKDDDDKASFNVLMVHRRSHTGERPLQCEICGFTCRQRGNLLRHIRLHTGERPFRCHLCNYACQRRDALSGSGSGSGMSASSQSPNPNNPAEYCSTIPPLQQAQASGALSSPPPTVMVPVGVLRHPGAEVAQPR

SEQ ID NO: 107 - DNA, 681 bp, synthetic construct
  misc_feature 1..681: note = Synthetic
  source 1..681: mol_type = other DNA
aagggcggcggcggctccggcggcggcggctccacgctcagtcccgttaaccacagtcttgacttgcagccagtcacctacagtgagcctgccttctggtgctcaatagcctactatgagttgaatcaaagagtcggtgaaaccttccatgcttcacaaccaagcctgactgtcgacggattcactgacccaagtaatagcgaaaggttctgccttgggttgctgtccaatgtaaaccggaacgccacagtcgagatgacaagaaggcatatcggtcgaggagtgagactttactacattgggggtgaagtgttcgctgagtgcttgagtgactctgcgatattcgtacagtcacctaactgtaaccaacggtacggatggcaccctgccacagtttgtaaaataccccctggatgcaatctcaagattttcaacaatcaggagttcgctgcgctgttggcccaatctgtcaaccaaggtttcgaagcggtctaccaactgacgcgaatgtgtacgatacgcatgtcttttgtgaaggggtggggtgccgagtacaggcgacaaacggtcactagcacaccctgctggatcgagcttcatttgaacggaccccttcagtggctggataaggtactgacgcagatggggtcaccaagcgtgcgctgtagttctatgtcttaa

SEQ ID NO: 108 - protein, 226 aa, synthetic construct
  REGION 1..226: note = Synthetic
KGGGGSGGGGSTLSPVNHSLDLQPVTYSEPAFWCSIAYYELNQRVGETFHASQPSLTVDGFTDPSNSERFCLGLLSNVNRNATVEMTRRHIGRGVRLYYIGGEVFAECLSDSAIFVQSPNCNQRYGWHPATVCKIPPGCNLKIFNNQEFAALLAQSVNQGFEAVYQLTRMCTIRMSFVKGWGAEYRRQTVTSTPCWIELHLNGPLQWLDKVLTQMGSPSVRCSSMS

SEQ ID NO: 109 - DNA, 669 bp, synthetic construct
  misc_feature 1..669: note = Synthetic
  source 1..669: mol_type = other DNA
aagggcggcggcggctccggcggcggcggctccgctcataacaacttggatctgcagccggtaacctattgtgagcctgcgttttggtgttctattagctactatgaacttaatcaaagggtgggggaaacgttccacgcctctcagccctctatgactgtggacggctttaccgacccatcaaactccgagagattctgcctcgggttgcttagcaacgtgaatagaaatgcggcagtcgagctcacccgacggcatataggtagaggagtacgcttgtactacattggaggagaagtatttgcggaatgtctctccgactccgctatctttgtacaatcacctaactgcaaccaaagatacgggtggcacccggccacagtatgtaagataccccccgggtgtaacctcaagattttcaataatcaggagtttgccgcccttcttgcccaaagtgttaatcaaggctttgaagcggtgtatcagttgaccagaatgtgtacgatacgaatgtcatttgttaaagggtggggcgcagaataccggcgacaaactgtgacttcaacgccgtgttggattgagctgcaccttaacggtcccctccagtggctggacaaagtacttacgcagatggggagccccagcattaggtgctctagcgtaagttaa

SEQ ID NO: 110 - protein, 222 aa, synthetic construct
  REGION 1..222: note = Synthetic
KGGGGSGGGGSAHNNLDLQPVTYCEPAFWCSISYYELNQRVGETFHASQPSMTVDGFTDPSNSERFCLGLLSNVNRNAAVELTRRHIGRGVRLYYIGGEVFAECLSDSAIFVQSPNCNQRYGWHPATVCKIPPGCNLKIFNNQEFAALLAQSVNQGFEAVYQLTRMCTIRMSFVKGWGAEYRRQTVTSTPCWIELHLNGPLQWLDKVLTQMGSPSIRCSSVS

SEQ ID NO: 111 - DNA, 1257 bp, synthetic construct
  misc_feature 1..1257: note = Synthetic
  source 1..1257: mol_type = other DNA
atgagcgcgtcatcccagagccctaacccaaataatcccgcagaatactgcagcacaataccccctttgcagcaggcacaagctagtggcgctttgagcagcccaccgcccacggtcatggttcctgtgggagtccttaaacatccaggggctgaagtagcacagccccggggcagtggtagtggctccggagctccacgaaacggcacggtccacctttacttgacgaaaccgctttatacctccgcaccgtccctccagcacctgtgtcggctgacaatcaataagtgtacaggtgcgatatggggattgccgcttccgacccgccttaaagattacctcgaggaatacaaatttcaagtcggttcaggtgcaacgaacttctcattgttgaagcaagccggtgatgttgaggaaaatccgggtcctatggctttgcctgttactgcgcttcttttgcctttggcattgttgcttcacgccgccaggcccgagcagaagctgatcagcgaggaggacctggacatacagatgacgcaaacaacttccagtcttagcgctagcctgggggatcgagtcaccatatcttgcagggcgtcccaagacattagcaagtatctcaattggtatcaacagaaacctgatggaacagttaaacttctgatttaccacacgagtcgcctgcactccggtgtgccctccagattctccggctcaggaagtggaaccgactactctctcaccatctccaacctcgaacaagaagacatagctacatacttttgccaacaaggtaataccctcccctataccttcggtggaggcactaagctggagatcacagggagcacgtccgggtctggcaaaccggggagtggtgaggggtctacgaagggagaagtcaagcttcaggagtcaggacccggtcttgtagctcccagccaaagcctgtcagttacatgcacggtttccggtgtgtctttgccagattatggcgtatcttggattcgccaaccgcctagaaagggacttgagtggttgggtgtcatttggggatcagaaacaacttactataacagtgctcttaagtccaggttgactataatcaaggacaatagtaagtcccaagtttttctgaaaatgaattccctgcagacagatgacaccgctatctactactgtgccaagcactactattatgggggctcttatgctatggactattggggtcaggggacatcagttactgtttccagc

SEQ ID NO: 112 - protein, 419 aa, synthetic construct
  REGION 1..419: note = Synthetic
MSASSQSPNPNNPAEYCSTIPPLQQAQASGALSSPPPTVMVPVGVLKHPGAEVAQPRGSGSGSGAPRNGTVHLYLTKPLYTSAPSLQHLCRLTINKCTGAIWGLPLPTRLKDYLEEYKFQVGSGATNFSLLKQAGDVEENPGPMALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 113 - DNA, 1311 bp, synthetic construct
  misc_feature 1..1311: note = Synthetic
  source 1..1311: mol_type = other DNA
atgggaggccttgattacaaggacgacgacgacaaggctagcttcaatgtcttaatggttcatcggcgaagccatactggtgaacgcccattgcagtgcgaaatatgcggctttacctgccgccagcgcggtaacctcctccgccacattcgtctgcacacaggggaacggccttttcggtgtcacctctgcaactatgcatgccaaagaagagatgcgctcagcgggtcaggtagtggctccggaatgagcgcgtcatcccagagccctaacccaaataatcccgcagaatactgcagcacaataccccctttgcagcaggcacaagctagtggcgctttgagcagcccaccgcccacggtcatggttcctgtgggagtccttagacatccaggggctgaagtagcacagccccggggttcaggtgcaacgaacttctcattgttgaagcaagccggtgatgttgaggaaaatccgggtcctatggctttgcctgttactgcgcttcttttgcctttggcattgttgcttcacgccgccaggcccgagcagaagctgatcagcgaggaggacctggacatacagatgacgcaaacaacttccagtcttagcgctagcctgggggatcgagtcaccatatcttgcagggcgtcccaagacattagcaagtatctcaattggtatcaacagaaacctgatggaacagttaaacttctgatttaccacacgagtcgcctgcactccggtgtgccctccagattctccggctcaggaagtggaaccgactactctctcaccatctccaacctcgaacaagaagacatagctacatacttttgccaacaaggtaataccctcccctataccttcggtggaggcactaagctggagatcacagggagcacgtccgggtctggcaaaccggggagtggtgaggggtctacgaagggagaagtcaagcttcaggagtcaggacccggtcttgtagctcccagccaaagcctgtcagttacatgcacggtttccggtgtgtctttgccagattatggcgtatcttggattcgccaaccgcctagaaagggacttgagtggttgggtgtcatttggggatcagaaacaacttactataacagtgctcttaagtccaggttgactataatcaaggacaatagtaagtcccaagtttttctgaaaatgaattccctgcagacagatgacaccgctatctactactgtgccaagcactactattatgggggctcttatgctatggactattggggtcaggggacatcagttactgtttccagc

SEQ ID NO: 114 - protein, 437 aa, synthetic construct
  REGION 1..437: note = Synthetic
MGGLDYKDDDDKASFNVLMVHRRSHTGERPLQCEICGFTCRQRGNLLRHIRLHTGERPFRCHLCNYACQRRDALSGSGSGSGMSASSQSPNPNNPAEYCSTIPPLQQAQASGALSSPPPTVMVPVGVLRHPGAEVAQPRGSGATNFSLLKQAGDVEENPGPMALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 115 - DNA, 972 bp, synthetic construct
  misc_feature 1..972: note = Synthetic
  source 1..972: mol_type = other DNA
tactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccaacaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggggttcaggtgcaacgaacttctcattgttgaagcaagccggtgatgttgaggaaaatccgggtcctatgagcgcgtcatcccagagccctaacccaaataatcccgcagaatactgcagcacaataccccctttgcagcaggcacaagctagtggcgctttgagcagcccaccgcccacggtcatggttcctgtgggagtccttaaacatccaggggctgaagtagcacagccccggggcagtggtagtggctccggagctccacgaaacggcacggtccacctttacttgacgaaaccgctttatacctccgcaccgtccctccagcacctgtgtcggctgacaatcaataagtgtacaggtgcgatatggggattgccgcttccgacccgccttaaagattacctcgaggaatacaaatttcaagtctccggaggcggcggagagggcagaggaagtcttctaacatgcggtgacgtggaggagaatcccggccctaggatg

SEQ ID NO: 116 - protein, 324 aa, synthetic construct
  REGION 1..324: note = Synthetic
YCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMSASSQSPNPNNPAEYCSTIPPLQQAQASGALSSPPPTVMVPVGVLKHPGAEVAQPRGSGSGSGAPRNGTVHLYLTKPLYTSAPSLQHLCRLTINKCTGAIWGLPLPTRLKDYLEEYKFQVSGGGGEGRGSLLTCGDVEENPGPRM

SEQ ID NO: 117 - DNA, 1026 bp, synthetic construct
  misc_feature 1..1026: note = Synthetic
  source 1..1026: mol_type = other DNA
tactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccaacaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggggttcaggtgcaacgaacttctcattgttgaagcaagccggtgatgttgaggaaaatccgggtcctatgggaggccttgattacaaggacgacgacgacaaggctagcttcaatgtcttaatggttcatcggcgaagccatactggtgaacgcccattgcagtgcgaaatatgcggctttacctgccgccagcgcggtaacctcctccgccacattcgtctgcacacaggggaacggccttttcggtgtcacctctgcaactatgcatgccaaagaagagatgcgctcagcgggtcaggtagtggctccggaatgagcgcgtcatcccagagccctaacccaaataatcccgcagaatactgcagcacaataccccctttgcagcaggcacaagctagtggcgctttgagcagcccaccgcccacggtcatggttcctgtgggagtccttagacatccaggggctgaagtagcacagccccggtccggaggcggcggagagggcagaggaagtcttctaacatgcggtgacgtggaggagaatcccggccctaggatg

SEQ ID NO: 118 - protein, 342 aa, synthetic construct
  REGION 1..342: note = Synthetic
YCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMGGLDYKDDDDKASFNVLMVHRRSHTGERPLQCEICGFTCRQRGNLLRHIRLHTGERPFRCHLCNYACQRRDALSGSGSGSGMSASSQSPNPNNPAEYCSTIPPLQQAQASGALSSPPPTVMVPVGVLRHPGAEVAQPRSGGGGEGRGSLLTCGDVEENPGPRM

SEQ ID NO: 119 - DNA, 1770 bp, synthetic construct
  misc_feature 1..1770: note = Synthetic
  source 1..1770: mol_type = other DNA
atgcctgctgctaagagagtgaaactggatctcgagggaagcgagaatcccttggaaatgtttgaaaccgtgcctgtttggagaagacaaccggtgcgcgtactttcattgttcgaagacattaaaaaggagctcaccagccttgggttcctcgagagtggaagcgatcctggtcagttgaaacacgtggttgacgtcacagacacggtgaggaaggatgttgaggagtggggtccattcgatttggtctatggcgcaacccctccgcttggacatacgtgcgatagacctccgagctggtatcttttccagtttcacaggctgcttcaatacgcaagaccgaagccgggttcccctcggcccttcttctggatgtttgtcgataatctcgttttgaataaagaggatttggatgtagcatcccgattccttgaaatggaaccggtaacgatccccgatgttcatggtgggagcttgcagaacgctgtccgggtgtggagtaacatacctgccatccggtcaaggcactgggctctcgtaagtgaggaagaactctcactgctggcgcagaacaagcagtcttctaaattggcggcaaaatggcccaccaagcttgtgaaaaattgctttctgccccttcgagagtattttaagtactttagtactgaactcacatcatcactgggcagtggtagtggctccggagctccacgaaacggcacggtccacctttacttgacgaaaccgctttatacctccgcaccgtccctccagcacctgtgtcggctgacaatcaataagtgtacaggtgcgatatggggattgccgcttccgacccgccttaaagattacctcgaggaatacaaatttcaagtcggttcaggtgcaacgaacttctcattgttgaagcaagccggtgatgttgaggaaaatccgggtcctatggctttgcctgttactgcgcttcttttgcctttggcattgttgcttcacgccgccaggcccgagcagaagctgatcagcgaggaggacctggacatacagatgacgcaaacaacttccagtcttagcgctagcctgggggatcgagtcaccatatcttgcagggcgtcccaagacattagcaagtatctcaattggtatcaacagaaacctgatggaacagttaaacttctgatttaccacacgagtcgcctgcactccggtgtgccctccagattctccggctcaggaagtggaaccgactactctctcaccatctccaacctcgaacaagaagacatagctacatacttttgccaacaaggtaataccctcccctataccttcggtggaggcactaagctggagatcacagggagcacgtccgggtctggcaaaccggggagtggtgaggggtctacgaagggagaagtcaagcttcaggagtcaggacccggtcttgtagctcccagccaaagcctgtcagttacatgcacggtttccggtgtgtctttgccagattatggcgtatcttggattcgccaaccgcctagaaagggacttgagtggttgggtgtcatttggggatcagaaacaacttactataacagtgctcttaagtccaggttgactataatcaaggacaatagtaagtcccaagtttttctgaaaatgaattccctgcagacagatgacaccgctatctactactgtgccaagcactactattatgggggctcttatgctatggactattggggtcaggggacatcagttactgtttccagc

SEQ ID NO: 120 - protein, 590 aa, synthetic construct
  REGION 1..590: note = Synthetic
MPAAKRVKLDLEGSENPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSLGSGSGSGAPRNGTVHLYLTKPLYTSAPSLQHLCRLTINKCTGAIWGLPLPTRLKDYLEEYKFQVGSGATNFSLLKQAGDVEENPGPMALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 121 - DNA, 1485 bp, synthetic construct
  misc_feature 1..1485: note = Synthetic
  source 1..1485: mol_type = other DNA
tactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccaacaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggggttcaggtgcaacgaacttctcattgttgaagcaagccggtgatgttgaggaaaatccgggtcctatgcctgctgctaagagagtgaaactggatctcgagggaagcgagaatcccttggaaatgtttgaaaccgtgcctgtttggagaagacaaccggtgcgcgtactttcattgttcgaagacattaaaaaggagctcaccagccttgggttcctcgagagtggaagcgatcctggtcagttgaaacacgtggttgacgtcacagacacggtgaggaaggatgttgaggagtggggtccattcgatttggtctatggcgcaacccctccgcttggacatacgtgcgatagacctccgagctggtatcttttccagtttcacaggctgcttcaatacgcaagaccgaagccgggttcccctcggcccttcttctggatgtttgtcgataatctcgttttgaataaagaggatttggatgtagcatcccgattccttgaaatggaaccggtaacgatccccgatgttcatggtgggagcttgcagaacgctgtccgggtgtggagtaacatacctgccatccggtcaaggcactgggctctcgtaagtgaggaagaactctcactgctggcgcagaacaagcagtcttctaaattggcggcaaaatggcccaccaagcttgtgaaaaattgctttctgccccttcgagagtattttaagtactttagtactgaactcacatcatcactgggcagtggtagtggctccggagctccacgaaacggcacggtccacctttacttgacgaaaccgctttatacctccgcaccgtccctccagcacctgtgtcggctgacaatcaataagtgtacaggtgcgatatggggattgccgcttccgacccgccttaaagattacctcgaggaatacaaatttcaagtctccggaggcggcggagagggcagaggaagtcttctaacatgcggtgacgtggaggagaatcccggccctaggatg

SEQ ID NO: 122 - protein, 495 aa, synthetic construct
  REGION 1..495: note = Synthetic
YCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMPAAKRVKLDLEGSENPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSLGSGSGSGAPRNGTVHLYLTKPLYTSAPSLQHLCRLTINKCTGAIWGLPLPTRLKDYLEEYKFQVSGGGGEGRGSLLTCGDVEENPGPRM

SEQ ID NO: 123 - DNA, 1530 bp, synthetic construct
  misc_feature 1..1530: note = Synthetic
  source 1..1530: mol_type = other DNA
aagggcggcggcggctccggcggcggcggctccaagcccaagtcccaagcctgcgaaccatccgaacctgaaatcgagatcaaacttccgaaactgaggacgctcgacgtcttctctggctgcggaggactgtctgaaggcttccaccaagcgggtataagtgacacgctctgggcgatcgaaatgtgggaccctgctgcccaggcgtttaggttgaataaccctggttctacagtctttacagaagattgcaatatactcctcaaattggtgatggcaggagaaactactaactcacgagggcaacggctccctcagaagggagatgtcgagatgctttgcggggggccgccatgccaaggcttttccggaatgaaccgattcaattcaaggacatattctaagtttaaaaacagtttggtagtaagttttctgagctactgtgattactatcgaccaagatttttcctcctggagaacgtccggaattttgtaagtttcaagagaagcatggtcttgaaattgacccttcgctgcttggtgaggatggggtaccagtgtacttttggcgtgctccaagcgggacagtacggtgttgcccaaactcggcggcgagctatcatcctcgcagctgcacccggtgaaaagttgccgctttttcctgaaccgctgcacgtattcgcgcctagggcgtgtcagctttcagtcgttgtcgatgataagaaattcgtatcaaacatcacccgactctcttccggcccgtttcggacgattacagtgcgcgacacgatgtcagacttgcccgaggttaggaacggcgcctcagcactcgaaatctcatacaacggtgaaccacaatcatggttccagcggcagcttcgcggtgcacaatatcagcccatactccgagaccacatctgcaaggacatgagcgccctggtagcagctcggatgcggcatatacctcttgcgccgggctctgattggagagatctccctaacatagaggtccggctttctgatgggacgatggcccggaaactgcgatatacgcatcacgaccggaagaacggtcggagtagttctggtgccttgcgcggggtgtgctcttgcgtggaggccggaaaggcttgcgatcctgccgctcggcaattcaatacgctgattccttggtgccttccgcataccgggaataggcacaatcattgggctggactttatggaagactcgaatgggacggtttttttagtacaaccgttactaacccagaaccaatgggcaagcagggccgggttttgcacccagagcagcacagagtcgtatctgtgagggagtgcgctcgaagccagggcttcccagatacttacaggctctttggtaatattctggataaacaccgccaagtcggaaacgcggtcccaccgcctctcgccaaggccattggcctcgagatcaagctgtgtatgctcgccaaggctagggaatctgcttccgcaaagataaaggaagaagaagctgcgaaagattaa

SEQ ID NO: 124 - protein, 509 aa, synthetic construct
  REGION 1..509: note = Synthetic
KGGGGSGGGGSKPKSQACEPSEPEIEIKLPKLRTLDVFSGCGGLSEGFHQAGISDTLWAIEMWDPAAQAFRLNNPGSTVFTEDCNILLKLVMAGETTNSRGQRLPQKGDVEMLCGGPPCQGFSGMNRFNSRTYSKFKNSLVVSFLSYCDYYRPRFFLLENVRNFVSFKRSMVLKLTLRCLVRMGYQCTFGVLQAGQYGVAQTRRRAIILAAAPGEKLPLFPEPLHVFAPRACQLSVVVDDKKFVSNITRLSSGPFRTITVRDTMSDLPEVRNGASALEISYNGEPQSWFQRQLRGAQYQPILRDHICKDMSALVAARMRHIPLAPGSDWRDLPNIEVRLSDGTMARKLRYTHHDRKNGRSSSGALRGVCSCVEAGKACDPAARQFNTLIPWCLPHTGNRHNHWAGLYGRLEWDGFFSTTVTNPEPMGKQGRVLHPEQHRVVSVRECARSQGFPDTYRLFGNILDKHRQVGNAVPPPLAKAIGLEIKLCMLAKARESASAKIKEEEAAKD

SEQ ID NO: 125 - DNA, 945 bp, synthetic construct
  misc_feature 1..945: note = Synthetic
  source 1..945: mol_type = other DNA
aagggcggcggcggctccggcggcggcggctccacttccgacactggacttgaatacgaagccccgaagctctacccagctatcccagccgcacgccggagaccgattagggtcctctctctgtttgatggaatcgcaacaggatacttggtactgaaggaactcgggattaaagttgggaagtatgtagcgagtgaggtgtgcgaggaaagcattgctgttgggacagtaaaacacgaaggcaacattaagtacgtcaatgatgtaaggaatattactaaaaagaacatagaagaatgggggcctttcgacctcgtgataggaggcagtccatgcaatgatctctccaacgttaatcctgcaaggaagggcctgtatgaaggaacaggccggctgttcttcgagttttaccacttgctgaattactctcgaccaaaagaaggtgatgatagacccttcttctggatgtttgaaaacgttgtcgctatgaaggtcggggataagcgcgacatcagtcgattcttggagtgtaaccctgtgatgatagatgcaatcaaggttagtgccgcgcacagagcgagatatttctggggaaatctgccggggatgaaccgccccgtgatagctagtaagaacgataagttggagctccaagattgtcttgaatataataggatcgccaaattgaagaaggtccaaacaatcacaacgaaaagtaattccataaaacagggaaaaaatcaactcttccccgtcgttatgaacggcaaagaggatgttctttggtgcacggaactggaaaggatcttcgggtttccagtgcattatacagatgttagtaacatgggaaggggagcgcgccagaagctgttggggagatcctggagtgtaccagttattcgacacttgttcgcgcctttgaaagattactttgcttgtgaataa

SEQ ID NO: 126 - protein, 314 aa, synthetic construct
  REGION 1..314: note = Synthetic
KGGGGSGGGGSTSDTGLEYEAPKLYPAIPAARRRPIRVLSLFDGIATGYLVLKELGIKVGKYVASEVCEESIAVGTVKHEGNIKYVNDVRNITKKNIEEWGPFDLVIGGSPCNDLSNVNPARKGLYEGTGRLFFEFYHLLNYSRPKEGDDRPFFWMFENVVAMKVGDKRDISRFLECNPVMIDAIKVSAAHRARYFWGNLPGMNRPVIASKNDKLELQDCLEYNRIAKLKKVQTITTKSNSIKQGKNQLFPVVMNGKEDVLWCTELERIFGFPVHYTDVSNMGRGARQKLLGRSWSVPVIRHLFAPLKDYFACE

SEQ ID NO: 127 - DNA, 540 bp, synthetic construct
  misc_feature 1..540: note = Synthetic
  source 1..540: mol_type = other DNA
tactgtaggagtaagaggagcaggctcctgcacagtgactacatgaacatgactcctagaaggcctggacccacccgcaagcattaccagccctatgccccaccacgcgacttcgcagcctatcgctcccgcgtgaaattcagccgcagcgcagatgctccagcctaccaacaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtccggaggcggcggagagggcagaggaagtcttctaacatgcggtgacgtggaggagaatcccggccctaggatg

SEQ ID NO: 128 - protein, 180 aa, synthetic construct
  REGION 1..180: note = Synthetic
YCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSGGGGEGRGSLLTCGDVEENPGPRM

SEQ ID NO: 129 - DNA, 1482 bp, synthetic construct
  misc_feature 1..1482: note = Synthetic
  source 1..1482: mol_type = other DNA
tactgtaggagtaagaggagcaggctcctgcacagtgactacatgaacatgactcctagaaggcctggacccacccgcaagcattaccagccctatgccccaccacgcgacttcgcagcctatcgctcccgcgtgaaattcagccgcagcgcagatgctccagcctaccaacaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggggttcaggtgcaacgaacttctcattgttgaagcaagccggtgatgttgaggaaaatccgggtcctatgcctgctgctaagagagtgaaactggatctcgagggaagcgagaatcccttggaaatgtttgaaaccgtgcctgtttggagaagacaaccggtgcgcgtactttcattgttcgaagacattaaaaaggagctcaccagccttgggttcctcgagagtggaagcgatcctggtcagttgaaacacgtggttgacgtcacagacacggtgaggaaggatgttgaggagtggggtccattcgatttggtctatggcgcaacccctccgcttggacatacgtgcgatagacctccgagctggtatcttttccagtttcacaggctgcttcaatacgcaagaccgaagccgggttcccctcggcccttcttctggatgtttgtcgataatctcgttttgaataaagaggatttggatgtagcatcccgattccttgaaatggaaccggtaacgatccccgatgttcatggtgggagcttgcagaacgctgtccgggtgtggagtaacatacctgccatccggtcaaggcactgggctctcgtaagtgaggaagaactctcactgctggcgcagaacaagcagtcttctaaattggcggcaaaatggcccaccaagcttgtgaaaaattgctttctgccccttcgagagtattttaagtactttagtactgaactcacatcatcactgggcagtggtagtggctccggagctccacgaaacggcacggtccacctttacttgacgaaaccgctttatacctccgcaccgtccctccagcacctgtgtcggctgacaatcaataagtgtacaggtgcgatatggggattgccgcttccgacccgccttaaagattacctcgaggaatacaaatttcaagtctccggaggcggcggagagggcagaggaagtcttctaacatgcggtgacgtggaggagaatcccggccctaggatg

SEQ ID NO: 130 - protein, 494 aa, synthetic construct
  REGION 1..494: note = Synthetic
YCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMPAAKRVKLDLEGSENPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSLGSGSGSGAPRNGTVHLYLTKPLYTSAPSLQHLCRLTINKCTGAIWGLPLPTRLKDYLEEYKFQVSGGGGEGRGSLLTCGDVEENPGPRM

SEQ ID NO: 131 - DNA, 831 bp, synthetic construct
  misc_feature 1..831: note = Synthetic
  source 1..831: mol_type = other DNA
atggctttgcctgttactgcgcttcttttgcctttggcattgttgcttcacgccgccaggcccgagcagaagctgatcagcgaggaggacctgcaggtacagttgaaacaaagtggtccagggcttgttcagccttcccagagtcttagcatcacatgcacggtatcagggttttcccttacaaactacggagtccactgggtgcggcagtctccaggtaagggtttggaatggttgggggtaatctggtctggcggaaatacggactataacaccccatttacctctcgcctgtcaatcaataaagacaattcaaaatctcaggtgttcttcaagatgaatagcctccagtcaaacgacacagcaatatattactgcgccagagccctcacgtattatgattatgaatttgcttattggggtcaaggtactcttgtaacagtaagtgcagggggtgggggttcaggaggtggtggctccggaggaggcgggtctggaggcggaggtagcgatattctgcttacccaatcacccgttattcttagcgtgtccccgggtgaaagagtatcatttagttgcagggcctctcaatccatcggaacgaacatccattggtatcaacaacgcaccaatgggagcccgcggctcctcataaaatatgctagcgagtccatcagtggcataccgtcaagattcagcggatcaggtagcggaaccgattttacactctcaataaactcagttgaaagtgaggatattgctgattattattgtcaacagaataacaattggccgacaacttttggcgctggcaccaaacttgaactgaag

SEQ ID NO: 132 - protein, 277 aa, synthetic construct
  REGION 1..277: note = Synthetic
MALPVTALLLPLALLLHAARPEQKLISEEDLQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAGGGGSGGGGSGGGGSGGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 133 - DNA, 1260 bp, synthetic construct
  misc_feature 1..1260: note = Synthetic
  source 1..1260: mol_type = other DNA
atgagcgcgtcatcccagagccctaacccaaataatcccgcagaatactgcagcacaataccccctttgcagcaggcacaagctagtggcgctttgagcagcccaccgcccacggtcatggttcctgtgggagtccttaaacatccaggggctgaagtagcacagccccggggcagtggtagtggctccggagctccacgaaacggcacggtccacctttacttgacgaaaccgctttatacctccgcaccgtccctccagcacctgtgtcggctgacaatcaataagtgtacaggtgcgatatggggattgccgcttccgacccgccttaaagattacctcgaggaatacaaatttcaagtcggttcaggtgcaacgaacttctcattgttgaagcaagccggtgatgttgaggaaaatccgggtcctatggctttgcctgttactgcgcttcttttgcctttggcattgttgcttcacgccgccaggcccgagcagaagctgatcagcgaggaggacctgcaggtacagttgaaacaaagtggtccagggcttgttcagccttcccagagtcttagcatcacatgcacggtatcagggttttcccttacaaactacggagtccactgggtgcggcagtctccaggtaagggtttggaatggttgggggtaatctggtctggcggaaatacggactataacaccccatttacctctcgcctgtcaatcaataaagacaattcaaaatctcaggtgttcttcaagatgaatagcctccagtcaaacgacacagcaatatattactgcgccagagccctcacgtattatgattatgaatttgcttattggggtcaaggtactcttgtaacagtaagtgcagggggtgggggttcaggaggtggtggctccggaggaggcgggtctggaggcggaggtagcgatattctgcttacccaatcacccgttattcttagcgtgtccccgggtgaaagagtatcatttagttgcagggcctctcaatccatcggaacgaacatccattggtatcaacaacgcaccaatgggagcccgcggctcctcataaaatatgctagcgagtccatcagtggcataccgtcaagattcagcggatcaggtagcggaaccgattttacactctcaataaactcagttgaaagtgaggatattgctgattattattgtcaacagaataacaattggccgacaacttttggcgctggcaccaaacttgaactgaag

SEQ ID NO: 134 - protein, 420 aa, synthetic construct
  REGION 1..420: note = Synthetic
MSASSQSPNPNNPAEYCSTIPPLQQAQASGALSSPPPTVMVPVGVLKHPGAEVAQPRGSGSGSGAPRNGTVHLYLTKPLYTSAPSLQHLCRLTINKCTGAIWGLPLPTRLKDYLEEYKFQVGSGATNFSLLKQAGDVEENPGPMALPVTALLLPLALLLHAARPEQKLISEEDLQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAGGGGSGGGGSGGGGSGGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 135 - DNA, 867 bp, synthetic construct
  misc_feature 1..867: note = Synthetic
  source 1..867: mol_type = other DNA
atgcaaatcccacaggctccatggccagtagtctgggcggtcttgcagcttgggtggcgacctggttggtttcttgactcacctgaccgcccctggaatccacctactttttcacccgcgctgctggtggtgaccgagggcgataatgctactttcacgtgtagtttttcaaatacgtccgaatctttcgtattgaactggtaccgaatgtctccttctaatcaaaccgataaactcgcagcctttccagaagacagatcccaaccaggacaagactgtcgcttccgcgtgacccaacttcccaatggtcgcgatttccatatgagcgtagtcagagccagaaggaatgattccgggacttatttgtgtggggccatctctcttgcccccaaggctcagattaaagagagtctgagagcggagctgcgggtaacggaaagacgcgctgaagtaccgacagcgcatcctagcccttctccacgaccggccggccaatttcagactttggtggtaggtgtggtaggcggtctgttggggagcctggttctcctcgtgtgggtactggcagtgatctgttctcgcgccgcacgaggaaccatcggtgcaagaaggactggccaaccacttaaggaggacccatctgctgtgcctgttttttctgtagattatggagaactcgacttccaatggagagaaaaaactcccgaaccaccggtaccctgtgtccccgaacaaacggaatatgctacgatcgtgtttccaagcggtatggggacttccagtcccgcaaggcgagggtcagccgacggtccacggagtgcacaaccattgaggccagaagatggacactgttcctggccgctttaa

SEQ ID NO: 136 - protein, 288 aa, synthetic construct; identical to SEQ ID NO: 38
  REGION 1..288: note = Synthetic

SEQ ID NO: 137 - DNA, 870 bp, synthetic construct
  misc_feature 1..870: note = Synthetic
  source 1..870: mol_type = other DNA
atgaagactttgccggcgatgttgggaactggtaagttgttttgggtgttctttcttattccttatcttgacatttggaatatccacgggaaagagtcatgtgatgtccaactttatatcaagcgacaaagcgaacattccatccttgccggcgatccattcgagctggaatgccccgtaaagtattgcgcgaatcgccctcacgttacctggtgcaaattgaatggaacgacgtgtgttaaattggaggaccggcagacttcttggaaagaagagaaaaatatctcttttttcattctgcatttcgagccagttcttccgaatgataatggctcatatcggtgctccgccaacttccagagcaatctcatagagagccactccactacgctttatgtcactgacgttaagtcagcttcagagcgcccgagtaaagacgaaatggcatcacggccctggcttctgtatcgcttgctcccactcgggggattgccgctcctcatcacaacttgtttctgcttgttctgctgtttgcgaaggcaccaggggaaacaaaatgaattgtctgacacggcgggacgcgagataaacctggtggatgcgcatctgaagtcagaacagacagaagcaagtacccgccagaactcccaggtcctgctgtctgaaactggtatatacgacaacgaccccgacttgtgtttccggatgcaagagggatctgaagtctatagtaacccgtgcctcgaagagaataaaccgggcatcgtgtatgcctctctcaatcattccgttattggaccaaactctaggctcgcaagaaatgttaaagaagcacctacggagtatgcaagcatttgcgttcggtcctaa

SEQ ID NO: 138 - protein, 289 aa, synthetic construct
  REGION 1..289: note = Synthetic
MKTLPAMLGTGKLFWVFFLIPYLDIWNIHGKESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMASRPWLLYRLLPLGGLPLLITTCFCLFCCLRRHQGKQNELSDTAGREINLVDAHLKSEQTEASTRQNSQVLLSETGIYDNDPDLCFRMQEGSEVYSNPCLEENKPGIVYASLNHSVIGPNSRLARNVKEAPTEYASICVRS

SEQ ID NO: 139 - DNA, 1113 bp, synthetic construct
  misc_feature 1..1113: note = Synthetic
  source 1..1113: mol_type = other DNA
atgctcggccaagttgtaactctcatactccttcttctgctgaaagtttaccaagggaaggggtgccaaggttctgcggaccatgtagtgagcatctcaggagtcccgcttcagctccagccgaatagtatccaaacaaaggtggacagcatagcttggaagaagttgttgccctctcaaaatggctttcatcacatactgaaatgggaaaatggcagcttgccttcaaacacatccaatgacagattttcattcatagttaaaaacctttcccttcttattaaggcggctcagcagcaagactcaggactctactgtctcgaagtaacatccatcagtggaaaggtccagacggctacatttcaggtctttgtttttgagtctctcttgcctgacaaggttgaaaaaccgagactccaaggacagggtaaaattctggatcggggacgatgccaggtagcacttagttgcctcgtatctcgggatggaaacgtatcctatgcttggtaccgcggatcaaaactgatacaaactgctggaaatttgacctacctggacgaggaagttgatataaatggaactcacacttatacttgtaacgtttcaaatccggtcagctgggaatctcatacactcaatctcactcaagattgccaaaatgcgcatcaagagtttagattttggccgtttctcgtaataatcgtgatcctgtctgccctgttcctcggaacgctggcttgtttttgcgtgtggcggcgcaaaaggaaggaaaagcagagcgaaacgagcccgaaggagttccttacaatctatgaggacgtaaaagatttgaaaaccaggcggaaccatgaacaagaacagacctttccgggaggtggaagtaccatttactccatgatacaatcacaatcatccgcgcctacgtcccaagaacccgcctatactctgtattctctcattcagcctagccggaaaagtggtagccgaaagcggaaccactccccttcattcaactccactatttacgaagtaatcggcaaatctcagcccaaagcacaaaacccagcacgcctgtcaaggaaggagctggagaacttcgatgtttacagttaa

SEQ ID NO: 140 - protein, 370 aa, synthetic construct
  REGION 1..370: note = Synthetic
MLGQVVTLILLLLLKVYQGKGCQGSADHVVSISGVPLQLQPNSIQTKVDSIAWKKLLPSQNGFHHILKWENGSLPSNTSNDRFSFIVKNLSLLIKAAQQQDSGLYCLEVTSISGKVQTATFQVFVFESLLPDKVEKPRLQGQGKILDRGRCQVALSCLVSRDGNVSYAWYRGSKLIQTAGNLTYLDEEVDINGTHTYTCNVSNPVSWESHTLNLTQDCQNAHQEFRFWPFLVIIVILSALFLGTLACFCVWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 141 - DNA, 849 bp, synthetic construct
  misc_feature 1..849: note = Synthetic
  source 1..849: mol_type = other DNA
atggtccgctggtttcatagagacctgtccgggcttgatgcggagaccttgctgaagggtcgaggagttcatggcagcttcctcgctagaccttcacggaaaaatcagggggactttagtctttccgtgagagtgggtgaccaggttacgcacattaggatccaaaatagtggtgacttctatgatctttatggtggtgagaaattcgccacactcacagagcttgttgagtactatacccaacaacaaggggtgttgcaggaccgagatggaacgatcattcacctgaagtaccccctgaattgctccgatcctacgtccgaaaggtggtatcatggtcatatgtccggcggacaagccgagacacttctccaagctaagggtgagccgtggaccttcttggttagagagagcctgtcccagccaggtgatttcgtactgtccgttttgagtgaccagcccaaagctggaccgggtagcccattgagggtcacccatattaaggtcatgtgtgagggcgggcgatatacagtgggcggtctcgaaacctttgacagcttgaccgatctcgttgagcacttcaaaaaaacaggaatagaagaagcgagtggcgcgtttgtatatctgcgacaaccctattacgcaaccagggtcaatggcagtggtagtggctccggagctccacgaaacggcacggtccacctttacttgacgaaaccgctttatacctccgcaccgtccctccagcacctgtgtcggctgacaatcaataagtgtacaggtgcgatatggggattgccgcttccgacccgccttaaagattacctcgaggaatacaaatttcaagtc

SEQ ID NO: 142 - protein, 283 aa, synthetic construct
  REGION 1..283: note = Synthetic
MVRWFHRDLSGLDAETLLKGRGVHGSFLARPSRKNQGDFSLSVRVGDQVTHIRIQNSGDFYDLYGGEKFATLTELVEYYTQQQGVLQDRDGTIIHLKYPLNCSDPTSERWYHGHMSGGQAETLLQAKGEPWTFLVRESLSQPGDFVLSVLSDQPKAGPGSPLRVTHIKVMCEGGRYTVGGLETFDSLTDLVEHFKKTGIEEASGAFVYLRQPYYATRVNGSGSGSGAPRNGTVHLYLTKPLYTSAPSLQHLCRLTINKCTGAIWGLPLPTRLKDYLEEYKFQV

SEQ ID NO: 143 - DNA, 867 bp, synthetic construct
  misc_feature 1..867: note = Synthetic
  source 1..867: mol_type = other DNA
atgacgtcaaggcgctggttccacccgaacatcacgggagtcgaggcggagaacttgcttctcactagaggtgtagacgggtcctttctcgcccgaccttccaaaagtaatccaggggacttcacactgtccgtccggcgcaatggcgccgttacccacatcaaaattcaaaatacaggagattactatgatctctacggaggcgaaaagttcgcgacgctcgcagaactcgtacagtactatatggagcatcatggtcaactgaaagaaaagaatggcgacgttatcgaattgaaataccccttgaactgtgcggatcctacgtctgaacgatggttccatggtcatcttagtggcaaggaagctgagaagcttctcacggaaaaagggaagcatggttccttccttgttagggaaagtcaatctcatcccggggacttcgtcctgtcagtgagaactggagatgataagggcgagtctaatgatggtaaatctaaggtgacccatgtgatgattagatgccaagagttgaagtacgatgtgggaggaggcgagagattcgactcattgactgatctggtcgaacattataaaaagaaccccatggtagaaacgttgggtacagttctccagttgaaacagcctttgaacacgactcggataaatgctgcggagggcagtggtagtggctccggagctccacgaaacggcacggtccacctttacttgacgaaaccgctttatacctccgcaccgtccctccagcacctgtgtcggctgacaatcaataagtgtacaggtgcgatatggggattgccgcttccgacccgccttaaagattacctcgaggaatacaaatttcaagtc

SEQ ID NO: 144 - protein, 289 aa, synthetic construct
  REGION 1..289: note = Synthetic
MTSRRWFHPNITGVEAENLLLTRGVDGSFLARPSKSNPGDFTLSVRRNGAVTHIKIQNTGDYYDLYGGEKFATLAELVQYYMEHHGQLKEKNGDVIELKYPLNCADPTSERWFHGHLSGKEAEKLLTEKGKHGSFLVRESQSHPGDFVLSVRTGDDKGESNDGKSKVTHVMIRCQELKYDVGGGERFDSLTDLVEHYKKNPMVETLGTVLQLKQPLNTTRINAAEGSGSGSGAPRNGTVHLYLTKPLYTSAPSLQHLCRLTINKCTGAIWGLPLPTRLKDYLEEYKFQV

SEQ ID NO: 145 - protein, 130 aa, synthetic construct
  REGION 1..130: note = Synthetic
MGGLDYKDDDDKASDQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSS

SEQ ID NO: 146 - protein, 217 aa, synthetic construct
  REGION 1..217: note = Synthetic
MGSENPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSL

SEQ ID NO: 147 - protein, 198 aa, synthetic construct
  REGION 1..198: note = Synthetic
MHHRNDSQRLGKAGCPPEPSLQMANTNFLSTLSPEHCRPLAGECMNKLKCGAAEAEIMNLPERVGTFSAIPALGGISLPPGVIVMTALHSPAAASAAVTDSAFQIANLADCPQNHSSSSSSSSGGAGGANPAKKKRKRCGVCVPCKRLINCGVCSSCRNRKAGAAICKFRKCEELKKKPGTSLERTPVPSAEAFRWFF

SEQ ID NO: 148 - protein, 57 aa, synthetic construct
  REGION 1..57: note = Synthetic
MSASSQSPNPNNPAEYCSTIPPLQQAQASGALSSPPPTVMVPVGVLKHPGAEVAQPR

SEQ ID NO: 149 - protein, 218 aa, synthetic construct
  REGION 1..218: note = Synthetic
VRWFHRDLSGLDAETLLKGRGVHGSFLARPSRKNQGDFSLSVRVGDQVTHIRIQNSGDFYDLYGGEKFATLTELVEYYTQQQGVLQDRDGTIIHLKYPLNCSDPTSERWYHGHMSGGQAETLLQAKGEPWTFLVRESLSQPGDFVLSVLSDQPKAGPGSPLRVTHIKVMCEGGRYTVGGLETFDSLTDLVEHFKKTGIEEASGAFVYLRQPYYATRVN

SEQ ID NO: 150 - protein, 224 aa, synthetic construct
  REGION 1..224: note = Synthetic
TSRRWFHPNITGVEAENLLLTRGVDGSFLARPSKSNPGDFTLSVRRNGAVTHIKIQNTGDYYDLYGGEKFATLAELVQYYMEHHGQLKEKNGDVIELKYPLNCADPTSERWFHGHLSGKEAEKLLTEKGKHGSFLVRESQSHPGDFVLSVRTGDDKGESNDGKSKVTHVMIRCQELKYDVGGGERFDSLTDLVEHYKKNPMVETLGTVLQLKQPLNTTRINAAE

SEQ ID NO: 151 - protein, 262 aa, synthetic construct
  REGION 1..262: note = Synthetic
DPAEAVLQEKALKFMNSSEREDCNNGEPPRKIIPEKNSLRQTYNSCARLCLNQETVCLASTAMKTENCVAKTKLANGTSSMIVPKQRKLSASYEKEKELCVKYFEQWSESDQVEFVEHLISQMCHYQHGHINSYLKPMLQRDFITALPARGLDHIAENILSYLDAKSLCAAELVCKEWYRVTSDGMLWKKLIERMVRTDSLWRGLAERRGWGQYLFKNKPPDGNAPPNSFYRALYPKIIQDIETIESNWRCGRHSLQRIHCR

SEQ ID NO: 152 - protein, 176 aa, synthetic construct
  REGION 1..176: note = Synthetic
RLNFGDDIPSALRIAKKKRWNSIEERRIHQESELHSYLSRLIAAERERELEECQRNHEGDEDDSHVRAQQACIEAKHDKYMADMDELFSQVDEKRKKRDIPDYLCGKISFELMREPCITPSGITYDRKDIEEHLQRVGHFDPVTRSPLTQEQLIPNLAMKEVIDAFISENGWVEDY

SEQ ID NO: 153 - protein, 292 aa, synthetic construct
  REGION 1..292: note = Synthetic
CVPRSGLILSCICLYCGVLLPVLLPNLPFLTCLSMSTLESVTYLPEKGLYCQRLPSSRTHGGTESLKGKNTENMGFYGTLKMIFYKMKRKLDHGSEVRSFSLGKKPCKVSEYTSTTGLVPCSATPTTFGDLRAANGQGQQRRRITSVQPPTGLQEWLKMFQSWSGPEKLLALDELIDSCEPTQVKHMMQVIEPQFQRDFISLLPKELALYVLSFLEPKDLLQAAQTCRYWRILAEDNLLWREKCKEEGIDEPLHIKRRKVIKPGFIHSPWKSAYIRQHRIDTNWRRGELKSP

SEQ ID NO: 154 - protein, 57 aa, synthetic construct
  REGION 1..57: note = Synthetic
APRNGTVHLYLTKPLYTSAPSLQHLCRLTINKCTGAIWGLPLPTRLKDYLEEYKFQV

SEQ ID NO: 155 - protein, 204 aa, synthetic construct
  REGION 1..204: note = Synthetic
SVNISGQNTMNMVKVPECRLADELGGLWENSRFTDCCLCVAGQEFQAHKAILAARSPVFSAMFEHEMEESKKNRVEINDVEPEVFKEMMCFIYTGKAPNLDKMADDLLAAADKYALERLKVMCEDALCSNLSVENAAEILILADLHSADQLKTQAVDFINYHASDVLETSGWKSMVVSHPHLVAEAYRSLASAQCPFLGPPRKR

SEQ ID NO: 156 - protein, 63 aa, synthetic construct
  REGION 1..63: note = Synthetic
GTLPVYTLKERCLQVVRSLVKPENYRRLDIVRSLYEDLEDHPNVQKDLERLTQERIAHQRMGD

SEQ ID NO: 157 - protein, 95 aa, synthetic construct
  REGION 1..95: note = Synthetic
EQAPEDQGPQREPHNEWTLELLEELKNEAVRHFPRIWLHGLGQHIYETYGDTWAGVEAIIRILQQLLFIHFRIGCRHSRIGVTQQRRARNGASRS

SEQ ID NO: 158 - protein, 81 aa, synthetic construct
  REGION 1..81: note = Synthetic
ADPRERVPPGNSGEETIGEAFEWLDRTIEALNREAVNHLPRELIFQVWQRSWAYWHDEQGMSTSYTKYRYLCIMQKAVYIH

SEQ ID NO: 159 - protein, 31 aa, synthetic construct
  REGION 1..31: note = Synthetic
GPCCLDVTCAELRTMDSTVNFVSWHANRQLG

SEQ ID NO: 160 - protein, 60 aa, synthetic construct
  REGION 1..60: note = Synthetic
FNVLMVHRRSHTGERPLQCEICGFTCRQRGNLLRHIRLHTGERPFRCHLCNYACQRRDAL

SEQ ID NO: 161 - protein, 159 aa, synthetic construct
  REGION 1..159: note = Synthetic
MHRKHLQEIPDLSSNVATSFTWGWDSSKTSELLSGMGVSALEKEEPDSENIPQELLSNLGHPESPPRKRLKSKGSDKDFVIVRRPKLNRENFPGVSWDSLPDELLLGIFSCLCLPELLKVSGVCKRWYRLASDESLWQTLDLTGKNLGSAGSAAGSGEF

SEQ ID NO: 162 - DNA, 876 bp, synthetic construct
  misc_feature 1..876: note = Synthetic
  source 1..876: mol_type = other DNA
atgcctgctgctaagagagtgaaactggatctcgagggaagcgagaatcccttggaaatgtttgaaaccgtgcctgtttggagaagacaaccggtgcgcgtactttcattgttcgaagacattaaaaaggagctcaccagccttgggttcctcgagagtggaagcgatcctggtcagttgaaacacgtggttgacgtcacagacacggtgaggaaggatgttgaggagtggggtccattcgatttggtctatggcgcaacccctccgcttggacatacgtgcgatagacctccgagctggtatcttttccagtttcacaggctgcttcaatacgcaagaccgaagccgggttcccctcggcccttcttctggatgtttgtcgataatctcgttttgaataaagaggatttggatgtagcatcccgattccttgaaatggaaccggtaacgatccccgatgttcatggtgggagcttgcagaacgctgtccgggtgtggagtaacatacctgccatccggtcaaggcactgggctctcgtaagtgaggaagaactctcactgctggcgcagaacaagcagtcttctaaattggcggcaaaatggcccaccaagcttgtgaaaaattgctttctgccccttcgagagtattttaagtactttagtactgaactcacatcatcactgggcagtggtagtggctccggagctccacgaaacggcacggtccacctttacttgacgaaaccgctttatacctccgcaccgtccctccagcacctgtgtcggctgacaatcaataagtgtacaggtgcgatatggggattgccgcttccgacccgccttaaagattacctcgaggaatacaaatttcaagtc

SEQ ID NO: 163 - protein, 292 aa, synthetic construct
  REGION 1..292: note = Synthetic
MPAAKRVKLDLEGSENPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSLGSGSGSGAPRNGTVHLYLTKPLYTSAPSLQHLCRLTINKCTGAIWGLPLPTRLKDYLEEYKFQV

SEQ ID NO: 164 - protein, 11 aa, synthetic construct
  REGION 1..11: note = Synthetic
  VAR_SEQ 9..10: note = X is any amino acid
  VAR_SEQ 5: note = X is any amino acid
  VAR_SEQ 7: note = X is any amino acid
RPVAXVXPXXR

SEQ ID NO: 165 - protein, 5 aa, synthetic construct
  REGION 1..5: note = Synthetic
  SITE 4: note = misc_feature - Xaa can be any naturally occurring amino acid
  VAR_SEQ 4: note = X is a guest residue consisting of any amino acid except proline
VPGXG

SEQ ID NO: 166 - protein, 15 aa, synthetic construct
  REGION 1..15: note = Synthetic
GGRPSDSYGAPGGGN

SEQ ID NO: 167 - protein, 11 aa, synthetic construct
  REGION 1..11: note = Synthetic
AQTPSSQYGAP

SEQ ID NO: 168 - protein, 16 aa, synthetic construct
  REGION 1..16: note = Synthetic
ADADADADARARARAR

SEQ ID NO: 169 - protein, 17 aa, synthetic construct
  REGION 1..17: note = Synthetic
SIRELEARIRELELRIG

SEQ ID NO: 170 - protein, 29 aa, synthetic construct
  REGION 1..29: note = Synthetic
YGCVAALETKIAALETKKAALETKIAALC

SEQ ID NO: 171 - protein, 16 aa, synthetic construct
  REGION 1..16: note = Synthetic
DAEFRHDSGYEVHHQK

SEQ ID NO: 172 - protein, 9 aa, synthetic construct
  REGION 1..9: note = Synthetic
  VAR_SEQ 8: note = X is L, F, or I
  VAR_SEQ 3: note = X is any amino acid
  VAR_SEQ 5: note = X is any amino acid
  VAR_SEQ 7: note = X is any amino acid
  VAR_SEQ 9: note = X is any amino acid
GGXGXDXXX

SEQ ID NO: 173 - protein, 14 aa, synthetic construct
  REGION 1..14: note = Synthetic
IQQLKNQIKQLLKQ

SEQ ID NO: 174 - protein, 11 aa, synthetic construct
  REGION 1..11: note = Synthetic
CCCCGGGSRGD

SEQ ID NO: 175 - protein, 16 aa, synthetic construct
  REGION 1..16: note = Synthetic
EAALEAALELAAELAA

SEQ ID NO: 176 - protein, 16 aa, synthetic construct
  REGION 1..16: note = Synthetic
KAALKAALKLAAKLAA

SEQ ID NO: 177 - protein, 16 aa, synthetic construct
  REGION 1..16: note = Synthetic
KAALEAALKLAAELAA

SEQ ID NO: 178 - protein, 16 aa, synthetic construct
  REGION 1..16: note = Synthetic
EAALKAALELAAKLAA

SEQ ID NO: 179 - protein, 37 aa, synthetic construct
  REGION 1..37: note = Synthetic
CGGEIRALKYEIARLKQAAQAKIRALEQKIAALEGGC

SEQ ID NO: 180 - protein, 16 aa, synthetic construct
  REGION 1..16: note = Synthetic
EACARVAAACEAAARQ

SEQ ID NO: 181 - protein, 36 aa, synthetic construct
  REGION 1..36: note = Synthetic
RVIEKTNEKFHQIEKEFSEVEGRIQDLEKYVEDTKI

SEQ ID NO: 182 - protein, 15 aa, synthetic construct
  REGION 1..15: note = Synthetic
ELALKAKAELELKAG

SEQ ID NO: 183 - protein, 15 aa, synthetic construct
  REGION 1..15: note = Synthetic
ELLAKKALEAEALKG

SEQ ID NO: 184 - protein, 13 aa, synthetic construct
  REGION 1..13: note = Synthetic
EWAVVLVAEAKHQ

SEQ ID NO: 185 - protein, 14 aa, synthetic construct
  REGION 1..14: note = Synthetic
WGKIQKLKIAKVFK

SEQ ID NO: 186 - protein, 15 aa, synthetic construct
  REGION 1..15: note = Synthetic
KVIKCKAAVLWEEKK

SEQ ID NO: 187 - protein, 20 aa, synthetic construct
  REGION 1..20: note = Synthetic
IIPTAQETWLGVLTIMEHTV

SEQ ID NO: 188 - protein, 19 aa, synthetic construct
  REGION 1..19: note = Synthetic
LSGGIDVVAHELTHAVTDY

SEQ ID NO: 189 - protein, 18 aa, synthetic construct
  REGION 1..18: note = Synthetic
PAVHASLDKFLSSVSTVL

SEQ ID NO: 190 - protein, 15 aa, synthetic construct
  REGION 1..15: note = Synthetic
GYQCGTITAKNVTAN

SEQ ID NO: 191 - protein, 15 aa, synthetic construct
  REGION 1..15: note = Synthetic
VAEAKVAEAKVAEAK

SEQ ID NO: 192 - protein, 17 aa, synthetic construct
  REGION 1..17: note = Synthetic
ETATKAELLAKYEATHK

SEQ ID NO: 193 - protein, 17 aa, synthetic construct
  REGION 1..17: note = Synthetic
ETATKAELLAKZEATHK

SEQ ID NO: 194 - protein, 41 aa, synthetic construct
  REGION 1..41: note = Synthetic
  VAR_SEQ 14: note = X is D or N
  VAR_SEQ 19: note = X is E or Q
IGKLKEEIDKLNRXLDDMXDENEQLKQENKTLLKVVGKLTR

SEQ ID NO: 195 - protein, 14 aa, synthetic construct
  REGION 1..14: note = Synthetic
EIAQLEYEISQLEQ

SEQ ID NO: 196 - protein, 14 aa, synthetic construct
  REGION 1..14: note = Synthetic
KIAQLKYKISQLKQ

SEQ ID NO: 197 - protein, 21 aa, synthetic construct
  REGION 1..21: note = Synthetic
EIAQLEYEISQLEQEIQALES

SEQ ID NO: 198 - protein, 21 aa, synthetic construct
  REGION 1..21: note = Synthetic
KIQALKQKISQLKWKIQSLKQ

SEQ ID NO: 199 - protein, 21 aa, synthetic construct
  REGION 1..21: note = Synthetic
QATNRNTDGSTDYGILQINSR

SEQ ID NO: 200 - protein, 24 aa, synthetic construct
  REGION 1..24: note = Synthetic
KLEALYVLGFFGFFTLGIMLSYIR

SEQ ID NO: 201 - protein, 24 aa, synthetic construct
  REGION 1..24: note = Synthetic
KLEALYVLGFFGFFTLGIMLSYIR

SEQ ID NO: 202 - protein, 12 aa, synthetic construct
  REGION 1..12: note = Synthetic
FKFEFKFEFKFE

SEQ ID NO: 203 - protein, 12 aa, synthetic construct
  REGION 1..12: note = Synthetic
FKFQFKFQFKFQ

SEQ ID NO: 204 - protein, 20 aa, synthetic construct
  REGION 1..20: note = Synthetic
VKVKVKTKVPPTKVKTKVKV

SEQ ID NO: 205 - protein, 16 aa, synthetic construct
  REGION 1..16: note = Synthetic
FEFEFKFKFEFEFKFK

SEQ ID NO: 206 - protein, 20 aa, synthetic construct
  REGION 1..20: note = Synthetic
VKVKVKVKVPPTKVKVKVKV

SEQ ID NO: 207 - protein, 41 aa, synthetic construct
  REGION 1..41: note = Synthetic
EIAQHEKEIQAIEKKIAQHEYKIQAIEEKIAQHKEKIQAIK

SEQ ID NO: 208 - protein, 11 aa, synthetic construct
  REGION 1..11: note = Synthetic
QQKFQFQFEQQ

SEQ ID NO: 209 - protein, 11 aa, synthetic construct
  REGION 1..11: note = Synthetic
QQRFEWEFEQQ

SEQ ID NO: 210 - protein, 30 aa, synthetic construct
  REGION 1..30: note = Synthetic
CKQLEDKIEELLSKAACKQLEDKIEELLSK

SEQ ID NO: 211 - protein, 26 aa, synthetic construct
  REGION 1..26: note = Synthetic
GNDYEDRYYRENMYRYPNQVYYRPVC

SEQ ID NO: 212 - protein, 31 aa, synthetic construct
  REGION 1..31: note = Synthetic
PHPGGSNWGQPHPGGSNWGQPHPGGSNWGQG

SEQ ID NO: 213 - protein, 17 aa, synthetic construct
  REGION 1..17: note = Synthetic
BPGELAQKLEQALQKLA

SEQ ID NO: 214 - protein, 16 aa, synthetic construct
  REGION 1..16: note = Synthetic
RARADADARARADADA

SEQ ID NO: 215 - protein, 16 aa, synthetic construct
  REGION 1..16: note = Synthetic
AEAEAKAKAEAEAKAK

SEQ ID NO: 216 - protein, 12 aa, synthetic construct
  REGION 1..12: note = Synthetic
KLDLKLDLKLDL

SEQ ID NO: 217 - protein, 7 aa, synthetic construct
  REGION 1..7: note = Synthetic
GSGSGSG

SEQ ID NO: 218 - protein, 8 aa, synthetic construct
  REGION 1..8: note = Synthetic
SGSGSGSG

SEQ ID NO: 219 - protein, 4 aa, synthetic construct
  REGION 1..4: note = Synthetic
GGGS

SEQ ID NO: 220 - protein, 5 aa, synthetic construct
  REGION 1..5: note = Synthetic
GGGGS

SEQ ID NO: 221 - protein, 9 aa, synthetic construct
  REGION 1..9: note = Synthetic
GGSGGSGGS

SEQ ID NO: 222 - protein, 15 aa, synthetic construct
  REGION 1..15: note = Synthetic
GGGGSGGGGSGGGGS

SEQ ID NO: 223 - protein, 10 aa, synthetic construct
  REGION 1..10: note = Synthetic
GGGGSGGGGS

SEQ ID NO: 224 - protein, 20 aa, synthetic construct
  REGION 1..20: note = Synthetic
GGGGSGGGGSGGGGSGGGGS

SEQ ID NO: 225 - protein, 25 aa, synthetic construct
  REGION 1..25: note = Synthetic
GGGGSGGGGSGGGGSGGGGSGGGGS

SEQ ID NO: 226 - protein, 30 aa, synthetic construct
  REGION 1..30: note = Synthetic
GGGGSGGGGSGGGGSGGGGSGGGGSGGGGS

SEQ ID NO: 227 - protein, 35 aa, synthetic construct
  REGION 1..35: note = Synthetic
GGGGSGGGGSGGGGSGGGGSGGGGSGGGGSGGGGS

SEQ ID NO: 228 - protein, 40 aa, synthetic construct
  REGION 1..40: note = Synthetic
GGGGSGGGGSGGGGSGGGGSGGGGSGGGGSGGGGSGGGGS

SEQ ID NO: 229 - protein, 45 aa, synthetic construct
  REGION 1..45: note = Synthetic
GGGGSGGGGSGGGGSGGGGSGGGGSGGGGSGGGGSGGGGSGGGGS

SEQ ID NO: 230 - protein, 50 aa, synthetic construct
  REGION 1..50: note = Synthetic
GGGGSGGGGSGGGGSGGGGSGGGGSGGGGSGGGGSGGGGSGGGGSGGGGS

SEQ ID NO: 231 - protein, 55 aa, synthetic construct
  REGION 1..55: note = Synthetic
GGGGSGGGGSGGGGSGGGGSGGGGSGGGGSGGGGSGGGGSGGGGSGGGGSGGGGS

SEQ ID NO: 232 - protein, 60 aa, synthetic construct
  REGION 1..60: note = Synthetic
GGGGSGGGGSGGGGSGGGGSGGGGSGGGGSGGGGSGGGGSGGGGSGGGGSGGGGSGGGGS

SEQ ID NO: 233 - protein, 21 aa, synthetic construct
  REGION 1..21: note = Synthetic
GGSGGSGGSGGSGGSGGSGGS

SEQ ID NO: 234 - protein, 4 aa, synthetic construct
  REGION 1..4: note = Synthetic
GSGS

SEQ ID NO: 235 - protein, 6 aa, synthetic construct
  REGION 1..6: note = Synthetic
GSGSGS

SEQ ID NO: 236 - protein, 8 aa, synthetic construct
  REGION 1..8: note = Synthetic
GSGSGSGS

SEQ ID NO: 237 - protein, 10 aa, synthetic construct
  REGION 1..10: note = Synthetic
GSGSGSGSGS

SEQ ID NO: 238 - protein, 12 aa, synthetic construct
  REGION 1..12: note = Synthetic
GSGSGSGSGSGS

SEQ ID NO: 239 - protein, 14 aa, synthetic construct
  REGION 1..14: note = Synthetic
GSGSGSGSGSGSGS

SEQ ID NO: 240 - protein, 16 aa, synthetic construct
  REGION 1..16: note = Synthetic
GSGSGSGSGSGSGSGS

SEQ ID NO: 241 - protein, 18 aa, synthetic construct
  REGION 1..18: note = Synthetic
GSGSGSGSGSGSGSGSGS

SEQ ID NO: 242 - protein, 20 aa, synthetic construct
  REGION 1..20: note = Synthetic
GSGSGSGSGSGSGSGSGSGS

SEQ ID NO: 243 - protein, 5 aa, synthetic construct
  REGION 1..5: note = Synthetic
GGSGG

SEQ ID NO: 244 - protein, 8 aa, synthetic construct
  REGION 1..8: note = Synthetic
GGSGGGGG

SEQ ID NO: 245 - protein, 5 aa, synthetic construct
  REGION 1..5: note = Synthetic
GGSSG

SEQ ID NO: 246 - protein, 5 aa, synthetic construct
  REGION 1..5: note = Synthetic
EAAAK

SEQ ID NO: 247 - protein, 10 aa, synthetic construct
  REGION 1..10: note = Synthetic
EAAAKEAAAK

SEQ ID NO: 248 - protein, 15 aa, synthetic construct
  REGION 1..15: note = Synthetic
EAAAKEAAAKEAAAK

SEQ ID NO: 249 - protein, 46 aa, synthetic construct
  REGION 1..46: note = Synthetic
AEAAAKEAAAKEAAAKEAAAKALEAEAAAKEAAAKEAAAKEAAAKA

SEQ ID NO: 250 - protein, 5 aa, synthetic construct
  REGION 1..5: note = Synthetic
PAPAP

SEQ ID NO: 251 - protein, 12 aa, synthetic construct
  REGION 1..12: note = Synthetic
AEAAAKEAAAKA

SEQ ID NO: 252 - protein, 17 aa, synthetic construct
  REGION 1..17: note = Synthetic
  SITE 9..10: note = This sequences can be cleaved between amino acid #9 and amino acid #10
VSQTSKLTRAETVFPDV

SEQ ID NO: 253 - protein, 6 aa, synthetic construct
  REGION 1..6: note = Synthetic
  SITE 3..4: note = This sequences can be cleaved between amino acid #3 and amino acid #4
PLGLWA

SEQ ID NO: 254 - protein, 6 aa, synthetic construct
  REGION 1..6: note = Synthetic
  SITE 3..4: note = This sequences can be cleaved between amino acid #3 and amino acid #4
RVLAEA

SEQ ID NO: 255 - protein, 10 aa, synthetic construct
  REGION 1..10: note = Synthetic
  SITE 6..7: note = This sequences can be cleaved between amino acid #6 and amino acid #7
EDVVCCSMSY

SEQ ID NO: 256 - protein, 8 aa, synthetic construct
  REGION 1..8: note = Synthetic
  SITE 6..7: note = This sequences can be cleaved between amino acid #6 and amino acid #7
GGIEGRGS

SEQ ID NO: 257 - protein, 10 aa, synthetic construct
  REGION 1..10: note = Synthetic
  SITE 7..8: note = This sequences can be cleaved between amino acid #7 and amino acid #8
TRHRQPRGWE

SEQ ID NO: 258 - protein, 10 aa, synthetic construct
  REGION 1..10: note = Synthetic
  SITE 7..8: note = This sequences can be cleaved between amino acid #7 and amino acid #8
AGNRVRRSVG

SEQ ID NO: 259 - protein, 9 aa, synthetic construct
  REGION 1..9: note = Synthetic
  SITE 7..8: note = This sequences can be cleaved between amino acid #7 and amino acid #8
  SITE 8..9: note = This sequences can be cleaved between amino acid # 8 and amino acid #9
RRRRRRRRR

SEQ ID NO: 260 - protein, 4 aa, synthetic construct
  REGION 1..4: note = Synthetic
  SITE 4: note = This sequences can be cleaved after amino acid #4
GFLG

SEQ ID NO: 261 - protein, 12 aa, synthetic construct
  REGION 1..12: note = Synthetic
CRRRRRREAEAC

SEQ ID NO: 262 - protein, 12 aa, synthetic construct
  REGION 1..12: note = Synthetic
  SITE 7..8: note = This sequences can be cleaved between amino acid #7 and amino acid #8
CRRRRRREAEAC

SEQ ID NO: 263 - protein, 8 aa, synthetic construct
  REGION 1..8: note = Synthetic
  SITE 1: note = misc_feature - Xaa can be any naturally occurring amino acid
  SITE 3: note = misc_feature - Xaa can be any naturally occurring amino acid
  SITE 5: note = misc_feature - Xaa can be any naturally occurring amino acid
  SITE 7: note = misc_feature - Xaa can be any naturally occurring amino acid
XPXPXPXP

SEQ ID NO: 264 - protein, 10 aa, synthetic construct
  REGION 1..10: note = Synthetic
  SITE 1: note = misc_feature - Xaa can be any naturally occurring amino acid
  SITE 3: note = misc_feature - Xaa can be any naturally occurring amino acid
  SITE 5: note = misc_feature - Xaa can be any naturally occurring amino acid
  SITE 7: note = misc_feature - Xaa can be any naturally occurring amino acid
  SITE 9: note = misc_feature - Xaa can be any naturally occurring amino acid
XPXPXPXPXP

SEQ ID NO: 265 - protein, 12 aa, synthetic construct
  REGION 1..12: note = Synthetic
  SITE 1: note = misc_feature - Xaa can be any naturally occurring amino acid
  SITE 3: note = misc_feature - Xaa can be any naturally occurring amino acid
  SITE 5: note = misc_feature - Xaa can be any naturally occurring amino acid
  SITE 7: note = misc_feature - Xaa can be any naturally occurring amino acid
  SITE 9: note = misc_feature - Xaa can be any naturally occurring amino acid
  SITE 11: note = misc_feature - Xaa can be any naturally occurring amino acid
XPXPXPXPXPXP

SEQ ID NO: 266 - protein, 14 aa, synthetic construct
  REGION 1..14: note = Synthetic
  SITE 1: note = misc_feature - Xaa can be any naturally occurring amino acid
  SITE 3: note = misc_feature - Xaa can be any naturally occurring amino acid
  SITE 5: note = misc_feature - Xaa can be any naturally occurring amino acid
  SITE 7: note = misc_feature - Xaa can be any naturally occurring amino acid
  SITE 9: note = misc_feature - Xaa can be any naturally occurring amino acid
  SITE 11: note = misc_feature - Xaa can be any naturally occurring amino acid
  SITE 13: note = misc_feature - Xaa can be any naturally occurring amino acid
XPXPXPXPXPXPXP

SEQ ID NO: 267 - protein, 16 aa, synthetic construct
  REGION 1..16: note = Synthetic
  SITE 1: note = misc_feature - Xaa can be any naturally occurring amino acid
  SITE 3: note = misc_feature - Xaa can be any naturally occurring amino acid
  SITE 5: note = misc_feature - Xaa can be any naturally occurring amino acid
  SITE 7: note = misc_feature - Xaa can be any naturally occurring amino acid
  SITE 9: note = misc_feature - Xaa can be any naturally occurring amino acid
  SITE 11: note = misc_feature - Xaa can be any naturally occurring amino acid
  SITE 13: note = misc_feature - Xaa can be any naturally occurring amino acid
  SITE 15: note = misc_feature - Xaa can be any naturally occurring amino acid
XPXPXPXPXPXPXPXP

SEQ ID NO: 268 - protein, 16 aa, synthetic construct
  REGION 1..16: note = Synthetic
GGGGSLVPRGSGGGGS

SEQ ID NO: 269 - protein, 11 aa, synthetic construct
  REGION 1..11: note = Synthetic
GGSGGHMGSGG

SEQ ID NO: 270 - protein, 18 aa, synthetic construct
  REGION 1..18: note = Synthetic
GGGSEGGGSEGGGSEGGG

SEQ ID NO: 271 - protein, 8 aa, synthetic construct
  REGION 1..8: note = Synthetic
AAGAATAA

SEQ ID NO: 272 - protein, 5 aa, synthetic construct
  REGION 1..5: note = Synthetic
GGGGG

SEQ ID NO: 273 - protein, 11 aa, synthetic construct
  REGION 1..11: note = Synthetic
GSGGGTGGGSG

SEQ ID NO: 274 - protein, 10 aa, synthetic construct
  REGION 1..10: note = Synthetic
  REGION 2..4: note = misc_feature - Xaa can be any naturally occurring amino acid
  REGION 6..7: note = misc_feature - Xaa can be any naturally occurring amino acid
  SITE 9: note = misc_feature - Xaa can be any naturally occurring amino acid
LXXXLXXLXL

SEQ ID NO: 275 - protein, 4 aa, synthetic construct
  REGION 1..4: note = Synthetic
KDEL

SEQ ID NO: 276 - protein, 15 aa, synthetic construct
  REGION 1..15: note = Synthetic
GGGGGGGGGGGGGGG

SEQ ID NO: 277 - protein, 15 aa, synthetic construct
  REGION 1..15: note = Synthetic
GGGGSGGGGSGGGGS

SEQ ID NO: 278 - protein, 5 aa, synthetic construct
  REGION 1..5: note = Synthetic
TEFCA

SEQ ID NO: 279 - protein, 5 aa, synthetic construct
  REGION 1..5: note = Synthetic
APRPG

SEQ ID NO: 280 - protein, 4 aa, synthetic construct
  REGION 1..4: note = Synthetic
GALA

SEQ ID NO: 281 - protein, 5 aa, synthetic construct
  REGION 1..5: note = Synthetic
RGDYC

SEQ ID NO: 282 - protein, 5 aa, synthetic construct
  REGION 1..5: note = Synthetic
ASSHN

SEQ ID NO: 283 - protein, 6 aa, synthetic construct
  REGION 1..6: note = Synthetic
KCCYSL

SEQ ID NO: 284 - protein, 7 aa, synthetic construct
  REGION 1..7: note = Synthetic
YEVGHRC

SEQ ID NO: 285 - protein, 8 aa, synthetic construct
  REGION 1..8: note = Synthetic
GPLGIAGQ

SEQ ID NO: 286 - protein, 107 aa, synthetic construct
  REGION 1..107: note = Synthetic
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 287 - protein, 9 aa, synthetic construct
  REGION 1..9: note = Synthetic
IEVMYPPPY

SEQ ID NO: 288 - protein, 128 aa, synthetic construct
  REGION 1..128: note = Synthetic
MAQVQLVESGGRLVQAGDSLRLSCAASGRTFSTSAMAWFRQAPGREREFVAAITWTVGNTILGDSVKGRFTISRDRAKNTVDLQMDNLEPEDTAVYYCSARSRGYVLSVLRSVDSYDYWGQGTQVTVS

SEQ ID NO: 289 - protein, 128 aa, synthetic construct
  REGION 1..128: note = Synthetic
MADVQLVESGGGLVQAGGSLRLSCAASGRTISMAAMSWFRQAPGKEREFVAGISRSAGSAVHADSVKGRFTISRDNTKNTLYLQMNSLKAEDTAVYYCAVRTSGFFGSIPRTGTAFDYWGQGTQVTVS

SEQ ID NO: 290 - protein, 128 aa, synthetic construct
  REGION 1..128: note = Synthetic
MAQVQLVESGGGLVQTGGSLKLSCTASVRTLSYYHVGWFRQAPGKEREFVAGIHRSGESTFYADSVKGRFTISRDNAKNTVHLQMNSLKPEDTAVYYCAQRVRGFFGPLRSTPSWYDYWGQGTQVTVS

SEQ ID NO: 291 - protein, 121 aa, synthetic construct
  REGION 1..121: note = Synthetic
MADVQLVESGGGLVQAGGSLRLSCAASGPTGAMAWFRQAPGKEREFVGGISGSETDTYYVDSVKGRFTVDRDNVKNTVYLQMNSLKPEDTAVYYCAARRRITLFTSRTDYDFWGRGTQVTV

SEQ ID NO: 292 - protein, 126 aa, synthetic construct
  REGION 1..126: note = Synthetic
MADVQLQESGGGLVQAGGSLRLSCAASGGTFSILSLGWFRQAPGKEREFVAAISRSEGSTDYADFVKGRFMISRENAKNTAYLQMNSLKPEDTAVYFCAASYARRLSTTASRVLYWGQGTQVTVSS

SEQ ID NO: 293 - protein, 253 aa, synthetic construct
  REGION 1..253: note = Synthetic
MSDPRERIPPGNSGEETIGEAFEWLNRTVEEINREAVNHLPRELIFQVWQRSWEYWHDEQGMSPSYVKYRYLCLIQKALFMHCKKGCRCLGEGHGAGGWRPGPPPPPPPGLAGGGGSGGGGSGGGGSMADVQLQESGGGLVQAGGSLRLSCAASGGTFSILSLGWFRQAPGKEREFVAAISRSEGSTDYADFVKGRFMISRENAKNTAYLQMNSLKPEDTAVYFCAASYARRLSTTASRVLYWGQGTQVTVSS

SEQ ID NO: 294 - protein, 255 aa, synthetic construct
  REGION 1..255: note = Synthetic
MSDPRERIPPGNSGEETIGEAFEWLNRTVEEINREAVNHLPRELIFQVWQRSWEYWHDEQGMSPSYVKYRYLCLIQKALFMHCKKGCRCLGEGHGAGGWRPGPPPPPPPGLAGGGGSGGGGSGGGGSMAQVQLVESGGRLVQAGDSLRLSCAASGRTFSTSAMAWFRQAPGREREFVAAITWTVGNTILGDSVKGRFTISRDRAKNTVDLQMDNLEPEDTAVYYCSARSRGYVLSVLRSVDSYDYWGQGTQVTVS

SEQ ID NO: 295 - protein, 253 aa, synthetic construct
  REGION 1..253: note = Synthetic
MADVQLQESGGGLVQAGGSLRLSCAASGGTFSILSLGWFRQAPGKEREFVAAISRSEGSTDYADFVKGRFMISRENAKNTAYLQMNSLKPEDTAVYFCAASYARRLSTTASRVLYWGQGTQVTVSSGGGGSGGGGSGGGGSMSDPRERIPPGNSGEETIGEAFEWLNRTVEEINREAVNHLPRELIFQVWQRSWEYWHDEQGMSPSYVKYRYLCLIQKALFMHCKKGCRCLGEGHGAGGWRPGPPPPPPPGLA

SEQ ID NO: 296 - protein, 255 aa, synthetic construct
  REGION 1..255: note = Synthetic
MAQVQLVESGGRLVQAGDSLRLSCAASGRTFSTSAMAWFRQAPGREREFVAAITWTVGNTILGDSVKGRFTISRDRAKNTVDLQMDNLEPEDTAVYYCSARSRGYVLSVLRSVDSYDYWGQGTQVTVSGGGGSGGGGSGGGGSMSDPRERIPPGNSGEETIGEAFEWLNRTVEEINREAVNHLPRELIFQVWQRSWEYWHDEQGMSPSYVKYRYLCLIQKALFMHCKKGCRCLGEGHGAGGWRPGPPPPPPPGLA

SEQ ID NO: 297 - DNA, 1497 bp, synthetic construct
  misc_feature 1..1497: note = Synthetic
  source 1..1497: mol_type = other DNA
tactgtaggagtaagaggagcaggctcctgcacagtgactacatgaacatgactcctagaaggcctggacccacccgcaagcattaccagccctatgccccaccacgcgacttcgcagcctatcgctcccgcgtgaaattcagccgcagcgcagatgctccagcctaccaacaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggggttcaggtgcaacgaacttctcattgttgaagcaagccggtgatgttgaggaaaatccgggtcctatggccccaaagaagaagcggaaggtcggtatccacggagtcccagcagccggaagcgagaatcccttggaaatgtttgaaaccgtgcctgtttggagaagacaaccggtgcgcgtactttcattgttcgaagacattaaaaaggagctcaccagccttgggttcctcgagagtggaagcgatcctggtcagttgaaacacgtggttgacgtcacagacacggtgaggaaggatgttgaggagtggggtccattcgatttggtctatggcgcaacccctccgcttggacatacgtgcgatagacctccgagctggtatcttttccagtttcacaggctgcttcaatacgcaagaccgaagccgggttcccctcggcccttcttctggatgtttgtcgataatctcgttttgaataaagaggatttggatgtagcatcccgattccttgaaatggaaccggtaacgatccccgatgttcatggtgggagcttgcagaacgctgtccgggtgtggagtaacatacctgccatccggtcaaggcactgggctctcgtaagtgaggaagaactctcactgctggcgcagaacaagcagtcttctaaattggcggcaaaatggcccaccaagcttgtgaaaaattgctttctgccccttcgagagtattttaagtactttagtactgaactcacatcatcactgggcagtggtagtggctccggagctccacgaaacggcacggtccacctttacttgacgaaaccgctttatacctccgcaccgtccctccagcacctgtgtcggctgacaatcaataagtgtacaggtgcgatatggggattgccgcttccgacccgccttaaagattacctcgaggaatacaaatttcaagtctccggaggcggcggagagggcagaggaagtcttctaacatgcggtgacgtggaggagaatcccggccctaggatg

SEQ ID NO: 298 - protein, 499 aa, synthetic construct
  REGION 1..499: note = Synthetic
YCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMAPKKKRKVGIHGVPAAGSENPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSLGSGSGSGAPRNGTVHLYLTKPLYTSAPSLQHLCRLTINKCTGAIWGLPLPTRLKDYLEEYKFQVSGGGGEGRGSLLTCGDVEENPGPRM

SEQ ID NO: 299 - DNA, 1512 bp, synthetic construct
  misc_feature 1..1512: note = Synthetic
  source 1..1512: mol_type = other DNA
tactgtaggagtaagaggagcaggctcctgcacagtgactacatgaacatgactcctagaaggcctggacccacccgcaagcattaccagccctatgccccaccacgcgacttcgcagcctatcgctcccgcgtgaaattcagccgcagcgcagatgctccagcctaccaacaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggggttcaggtgcaacgaacttctcattgttgaagcaagccggtgatgttgaggaaaatccgggtcctatgggaagcgagaatcccttggaaatgtttgaaaccgtgcctgtttggagaagacaaccggtgcgcgtactttcattgttcgaagacattaaaaaggagctcaccagccttgggttcctcgagagtggaagcgatcctggtcagttgaaacacgtggttgacgtcacagacacggtgaggaaggatgttgaggagtggggtccattcgatttggtctatggcgcaacccctccgcttggacatacgtgcgatagacctccgagctggtatcttttccagtttcacaggctgcttcaatacgcaagaccgaagccgggttcccctcggcccttcttctggatgtttgtcgataatctcgttttgaataaagaggatttggatgtagcatcccgattccttgaaatggaaccggtaacgatccccgatgttcatggtgggagcttgcagaacgctgtccgggtgtggagtaacatacctgccatccggtcaaggcactgggctctcgtaagtgaggaagaactctcactgctggcgcagaacaagcagtcttctaaattggcggcaaaatggcccaccaagcttgtgaaaaattgctttctgccccttcgagagtattttaagtactttagtactgaactcacatcatcactgggcagtggtagtggctccggagctccacgaaacggcacggtccacctttacttgacgaaaccgctttatacctccgcaccgtccctccagcacctgtgtcggctgacaatcaataagtgtacaggtgcgatatggggattgccgcttccgacccgccttaaagattacctcgaggaatacaaatttcaagtcggcggcggcggctccaagcgtcctgctgctactaagaaagctggtcaagctaagaaaaagaaatccggaggcggcggagagggcagaggaagtcttctaacatgcggtgacgtggaggagaatcccggccctaggatg

SEQ ID NO: 300 - protein, 504 aa, synthetic construct
  REGION 1..504: note = Synthetic
YCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMGSENPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSLGSGSGSGAPRNGTVHLYLTKPLYTSAPSLQHLCRLTINKCTGAIWGLPLPTRLKDYLEEYKFQVGGGGSKRPAATKKAGQAKKKKSGGGGEGRGSLLTCGDVEENPGPRM

SEQ ID NO: 301 - DNA, 453 bp, synthetic construct
  misc_feature 1..453: note = Synthetic
  source 1..453: mol_type = other DNA
aaactggggcacaagcttaatggttcaggtgcaacgaacttctcattgttgaagcaagccggtgatgttgaggaaaatccgggtcctatgagcgcgtcatcccagagccctaacccaaataatcccgcagaatactgcagcacaataccccctttgcagcaggcacaagctagtggcgctttgagcagcccaccgcccacggtcatggttcctgtgggagtccttaaacatccaggggctgaagtagcacagccccggggcagtggtagtggctccggagctccacgaaacggcacggtccacctttacttgacgaaaccgctttatacctccgcaccgtccctccagcacctgtgtcggctgacaatcaataagtgtacaggtgcgatatggggattgccgcttccgacccgccttaaagattacctcgaggaatacaaatttcaagtctaa

SEQ ID NO: 302 - protein, 150 aa, synthetic construct
  REGION 1..150: note = Synthetic
KLGHKLNGSGATNFSLLKQAGDVEENPGPMSASSQSPNPNNPAEYCSTIPPLQQAQASGALSSPPPTVMVPVGVLKHPGAEVAQPRGSGSGSGAPRNGTVHLYLTKPLYTSAPSLQHLCRLTINKCTGAIWGLPLPTRLKDYLEEYKFQV

SEQ ID NO: 303 - DNA, 6951 bp, synthetic construct
  misc_feature 1..6951: note = Synthetic
  source 1..6951: mol_type = other DNA
aggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaaccctggaattcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaaactagtaagcttggcgtaactagatcttgagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttgggctcgagggggcccgggtgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtacggccaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgccgtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtacgtatagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagc

SEQ ID NO: 304 - DNA, 10573 bp, synthetic construct
  misc_feature 1..10573: note = Synthetic
  source 1..10573: mol_type = other DNA
cgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgactcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggatcgataccgtcgactccggaatagccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggagacggacgtctcataaatgcatgagtaactgaggatccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaaccctggaattcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaaactagtaagcttggcgtaactagatcttgagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttgggctcgagggggcccgggtgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtacggccaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgccgtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtacgtatagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagc

SEQ ID NO: 305 - DNA, 7784 bp, synthetic construct
  misc_feature 1..7784: note = Synthetic
  source 1..7784: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgcgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttagtaccgggcccgctctagccgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagcggccgctgagttaactattctagagccaccatggagacggacgtctcaggttcaggtgcaacgaacttctcattgttgaagcaagccggtgatgttgaggaaaatccgggtcctatggtgtctaagggcgaagagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagaccctgtaccccgctgacggcggcctggaaggcagaaacgacatggccctgaagctcgtgggcgggagccatctgatcgcaaacgccaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaaaagtaaacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgaagtaaagcggccgcatcgataccgtcgacctcgatcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 306 - DNA, 8450 bp, synthetic construct
  misc_feature 1..8450: note = Synthetic
  source 1..8450: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgcgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttagtaccgggcccgctctagccgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagcggccgctgagttaactattctagagccaccatggagacggacgtctcaacaactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccaacaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtccggaggcggcggagagggcagaggaagtcttctaacatgcggtgacgtggaggagaatcccggccctaggatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaaacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgaagtaaagcggccgcatcgataccgtcgacctcgatcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

(19) United States
(12) Patent Application Publication
(10) Pub. No.: US 2025/0241955 A1
(43) Pub. Date: Jul. 31, 2025

(54) ENGINEERED BIFUNCTIONAL RECEPTORS AND USES THEREOF

(71) Applicants: THE GENERAL HOSPITAL CORPORATION, Boston, MA (US); DANA-FARBER CANCER INSTITUTE, INC., Boston, MA (US)

(72) Inventors: MAX JAN, Boston, MA (US); BENJAMIN EBERT, Boston, MA (US); MARCELA MAUS, Boston, MA (US); ROBERT MANGUSO, Boston, MA (US); KEPLER MEARS, Boston, MA (US); ISABEL LANE, Boston, MA (US)

(73) Assignees: THE GENERAL HOSPITAL CORPORATION, Boston, MA (US); DANA-FARBER CANCER INSTITUTE, INC., Boston, MA (US)

(21) Appl. No.: 19/175,969

(22) Filed: Apr. 10, 2025

Related U.S. Application Data
(63) Continuation of application No. PCT/US2023/076534, filed on Oct. 11, 2023.
(60) Provisional application No. 63/415,180, filed on Oct. 11, 2022.

(51) Int. Cl. A61K 35/17; A61K 40/11; A61K 40/31; A61K 40/42; C07K 16/28; C07K 16/40; C12N 15/86

(52) U.S. Cl. CPC A61K 35/17; A61K 40/11; A61K 40/31; A61K 40/4211; A61K 40/4244; C07K 16/2803; C07K 16/40; C12N 15/86

(57) ABSTRACT

Described in several example embodiments herein are engineered bifunctional receptors that can include an E3 ligase binding domain and a target binding domain operatively coupled to the E3 ligase binding domain. In some embodiments, the engineered bifunctional receptors are capable of targeted degradation of a target protein. Also described in several example embodiments herein are compositions, formulations, and cells that can include or generate the engineered bifunctional receptors and uses thereof.

### CROSS-REFERENCE TO RELATED APPLICATIONS

This application claims the benefit of and priority to U.S. Provisional Patent Application No. 63/415,180, filed on Oct. 11, 2022, the contents of which is incorporated by reference herein in its entirety.

### SEQUENCE LISTING

This application contains a sequence listing filed in electronic form as an xml file entitled “BROD-5695WP_ST26.xml”, created on Oct. 10, 2023, and having a size of 533,569 bytes. The content of the sequence listing is incorporated herein in its entirety.

### TECHNICAL FIELD

The subject matter disclosed herein is generally directed to engineered bifunctional receptors and uses thereof, such as for targeted protein degradation.

### BACKGROUND

Targeted protein depletion is desirable for clinical and non-clinical applications. In some contexts, the targeted protein elimination or reduction has been achieved using genetic knockout or knockdown approaches. However, such approaches are still limited and inappropriate in some contexts. For example, such approaches are irreversible, poorly controllable, and/or difficult to scale to simultaneously target multiple genes/proteins in a single cell. Thus, there exists a need for improved strategies for targeted protein reduction.

Citation or identification of any document in this application is not an admission that such a document is available as prior art to the present invention.

### SUMMARY

Described in several exemplary embodiments herein are engineered bifunctional receptors comprising an E3 ligase binding domain and a target binding domain operatively coupled to the E3 ligase binding domain.

In an example embodiment, the target binding domain is operatively coupled to the E3 ligase binding domain via a linker, optionally a flexible linker.

In an example embodiment, the engineered bifunctional receptor inducibly binds an E3 ligase in response to a stimulus. In an example embodiment, the stimulus is a chemical, biological, or physical stimulus. In an example embodiment, the chemical stimulus is thalidomide or a derivative thereof.

In an example embodiment, the E3 ligase binding domain is or comprises a zinc finger domain.

In an example embodiment, the engineered bifunctional receptor further comprises a stimulus responsive domain.

In an example embodiment, the E3 ligase binding domain is viral or eukaryotic. In an example embodiment the E3 ligase binding domain is human.

In an example embodiment, the engineered bifunctional receptor is configured for programmable and/or inducible degradation of one or more target polypeptides.

In an example embodiment, the target binding domain is capable of binding one or more target polypeptides. In an example embodiment, the target binding domain is capable of binding two or more target polypeptides. In an example embodiment, the target binding domain is capable of binding two or more different target polypeptides.

In an example embodiment, the E3 ligase binding domain comprises or consists of a sequence according to SEQ ID NO: 78, 151, 152, 153, 154, 155, 156, 157, 158, 159, 160, or 161; the target binding domain comprises or consists of a sequence according SEQ ID NO: 38, 46, 48, 50, 76, 108, 110, 124, 132, 136, 138, 140, 145, 146, 147, 148, 149, 150; and/or any combination thereof.

In an example embodiment, the one or more target polypeptides or two or more different target polypeptides are bound by the target binding domain and are endogenously expressed in a cell-based therapeutic and are associated with decreasing or otherwise interfering with a therapeutic efficacy of the cell-based therapeutic.

In an example embodiment, the cell-based therapeutic is a chimeric antigen receptor (CAR) T cell, T cell receptor (TCR) T cells, tumor infiltrating lymphocyte, B-cell, a Natural Killer (NK) cell, a CAR-NK cell, a stem cell, or an induced pluripotent stem cell (iPSC).

In an example embodiment, the one or more target polypeptides or the two or more different target polypeptides mediate cellular dysfunction, exhaustion, transformation, or any combination thereof.

In an example embodiment, the one or more target polypeptides or the two or more different target polypeptides are expressed in a cell used for biomanufacturing and are associated with decreasing a yield of a bioproduct or otherwise interfere with the production of the bioproduct.

In an example embodiment, the target binding domain comprises an antibody or fragment thereof, nanobody, affibody, knottin, DARPin, adnectin, anticalin, avimer, Fynomer, beta-hairpin mimetics, alphabody, or any combination thereof.

In an example embodiment, the target binding domain comprises a protein receptor or receptor ligand.

Described in an example embodiment herein are engineered polynucleotides that encode the engineered bifunctional receptor of the present invention.

In an example embodiment, the engineered polynucleotides further comprise a regulatory element operatively coupled to the polynucleotide that encodes the engineered bifunctional receptor. In an example embodiment, the regulatory element is a constitutive promoter or an inducible promoter.

Described in an example embodiment herein are vectors or vector systems comprising the engineered polynucleotide of the present invention.

Described in an example embodiment herein are delivery vehicles comprising an engineered bifunctional receptor of the present invention, an engineered polynucleotide of the present invention, a vector or vector system of the present invention, or any combination thereof.

Described in an example embodiment herein are cells comprising an engineered bifunctional receptor of the present invention, an engineered polynucleotide of the present invention, a vector or vector system of the present invention, a delivery vehicle of the present invention, or any combination thereof. In an example embodiment, the cell is a cell for adoptive cell therapy. In an example embodiment, the cell is a chimeric antigen receptor (CAR) T cell, T cell receptor (TCR) T cells, tumor infiltrating lymphocyte, B-cell, or a Natural Killer cell.

Described in an example embodiment herein are cell populations comprising one or more cells of the present invention.

Described in an example embodiment herein are pharmaceutical formulations comprising an engineered bifunctional receptor of the present invention, an engineered polynucleotide of the present invention, a vector or vector system of the present invention, a delivery vehicle of the present invention, a cell or cell population of the present invention, or any combination thereof, and a pharmaceutically acceptable carrier.

Described in an example embodiment herein are kits comprising an engineered bifunctional receptor of the present invention, an engineered polynucleotide of the present invention, a vector or vector system of the present invention, a delivery vehicle of the present invention, a cell or cell population of the present invention, a pharmaceutical formulation of the present invention, or any combination thereof.

Described in an example embodiment herein are methods of targeted protein degradation, the method comprising delivering an engineered bifunctional receptor of any one of the present invention, an engineered polynucleotide of the present invention, the vector or vector system of the present invention, a delivery vehicle of the present invention, a pharmaceutical formulation of the present invention, or any combination thereof to a cell or cell population under conditions sufficient to permit binding of the target binding domain to one or more target polypeptides thereby triggering induced-proximity degradation of the one or more target polypeptides. In an example embodiment, the cell or cell population has a decreased amount of the one or more target polypeptides as compared to a suitable control cell. In an example embodiment, the cell or cell population is a cell for adoptive cell therapy. In an example embodiment, the cell is a chimeric antigen receptor (CAR) T cell, T cell receptor (TCR) T cells, tumor infiltrating lymphocyte, B-cell, or a Natural Killer cell. In an example embodiment, delivering occurs in vitro, in vivo, in situ, or ex vivo. In an example embodiment, the one or more target polypeptides are endogenous to the cell or cell population.

Described in an example embodiment herein are methods of treatment comprising delivering a cell or cell population of the present invention to a subject in need thereof. In an example embodiment, the cell is autologous or allogeneic.

In an example embodiment, the method further comprises delivering an engineered bifunctional receptor of the present invention, an engineered polynucleotide of the present invention, a vector or vector system of the present invention, a delivery vehicle of the present invention, a pharmaceutical formulation of the present invention, or any combination thereof to the cell or cell population under conditions sufficient to permit binding of the target binding domain to one or more target polypeptides thereby triggering induced-proximity degradation of the one or more target polypeptides. In an example embodiment, delivering occurs ex vivo. In an example embodiment, the one or more target polypeptides are endogenous to the cell or cell population. In an example embodiment, the one or more target polypeptides mediate cellular dysfunction, exhaustion, and/or transformation. In an example embodiment, the cell or cell population has a decreased amount of the one or more target polypeptides as compared to a suitable control cell. In an example embodiment, the subject in need thereof has a cancer.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1A-1H—Reprogramming E3 ligase specificity using synthetic substrate receptors (SSRs). (FIG. 1A) Schematic of the degradation system using a GFP-binding SSR. (FIG. 1B) Normalized GFP abundance in HEK293T cells dually transduced with GFP and a GFP degrader composed of the vhhGFP4 nanobody linked to the indicated ligase-binding domain. (FIG. 1C) GFP intensity in Jurkat cells transduced with GFP and/or the GFP degrader vhhGFP4-SOCS2. (FIG. 1D) Normalized GFP abundance in Jurkat cells treated with the neddylation inhibitor MLN4924. Cells were treated with 0.5 uM MLN4924 for 4 hours and analyzed by flow cytometry. (FIG. 1E) Normalized GFP abundance in multiple cell lines transduced with GFP and either the SOCS2- or Vpx-based GFP degrader. (FIG. 1F) Normalized abundance of untagged GFP in the nucleus/cytosol and cell surface-localized GFP fused to a type I transmembrane domain of Jurkat cells co-transduced with the SOCS2-based GFP degrader. (FIG. 1G) Schematic of a cell activation-inducible degradation system using a CD19-activated SynNotch receptor. (FIG. 1H) Normalized GFP abundance in Jurkat cells transduced to express GFP and either a control SynNotch receptor or a SynNotch-degrader construct. Jurkats were stimulated with CD19+ target cells overnight and analyzed via flow cytometry. Two-sided student's t-tests were performed as indicated. Experiments were performed in technical duplicate; data represents one of two independent experiments.

FIG. 2A-2E-Engineering a lenalidomide-inducible SSR system. (FIG. 2A) Schematic of the lenalidomide-inducible degradation system using a GFP-binding SSR and a zinc finger ligase-binding domain. (FIG. 2B) GFP intensity in Jurkat cells dually transduced with GFP and the lenalidomide-inducible GFP degrader ZnF-vhhGFP4. Cells were incubated with or without 1000 nM lenalidomide for two hours and analyzed via flow cytometry. (FIG. 2C) Normalized GFP abundance in Jurkat cells dually transduced with GFP and the lenalidomide-inducible GFP degrader. (FIG. 2D) Timecourse assay showing normalized GFP degradation in Jurkat cells dually transduced with GFP and a GFP-targeted SSR and treated with 1000 nM lenalidomide. (FIG. 2E) Dose response assay showing normalized GFP degradation in dually transduced Jurkat cells treated overnight with 1000 nM lenalidomide.

FIG. 3A-3K-Targeted degradation of cell therapy-relevant endogenous proteins. (FIG. 3A) Schematic of the degradation system using a SARA-based SSR to bind SMAD2/3. (FIG. 3B) Schematic of SMAD2/3-dependent signaling in the TGFβ pathway. (FIG. 3C) Normalized GFP abundance in Jurkat cells dually transduced with a SMAD2- or SMAD3-GFP reporter and SOCS2-based or zinc finger lenalidomide-inducible degrader. ZnF-SARA Jurkat cells were incubated overnight with or without 1000 nM lenalidomide. (FIG. 3D) Flow plot showing GFP and mTagBFP intensity in Jurkat cells dually transduced with either a SMAD2- or SMAD3-GFP reporter and a SARA-based degrader. (FIG. 3E) Endogenous SMAD2/3 intensity in Jurkat cells transduced with the SARA-based degrader. Cells were analyzed via intracellular flow (ICF). (FIG. 3F) Mean fluorescent intensity (MFI) of SMAD2/3 in Jurkat cells transduced with the SARA-based degrader. (FIG. 3G) Normalized levels of SMAD reporter in HEKBlue cells transduced with the SARA-based degrader. (FIG. 3H) Schematic of the degradation system using a DNMT3L-based SSR to degrade DNMT3A. (FIG. 3I) Flow plot showing GFP and mTagBFP intensity in Jurkat cells dually transduced with either a DNMT3A- or DNMT3B-GFP reporter and a DNMT3L-based degrader. (FIG. 3J) Normalized GFP in Jurkat cells dually transduced with a DNMT3A-GFP reporter and either a SOCS2-based or lenalidomide-inducible zinc finger-based degrader. (FIG. 3K) Western blot showing endogenous DNMT3A degradation in Jurkat cells transduced with a DNMT3L-based degrader containing a variable nuclear localization signal.

FIG. 4—Shows a strategy and results of an exemplary proteomic and CRISPR-based screen for characterizing and analyzing specificity of an engineered bifunctional receptor described herein. FIG. 4 shows an example that tests the specificity of SMAD and DNMT3A targeting engineered bifunctional receptors.

FIG. 5A-5F—SMAD degradation enhanced CAR T cell proliferation and anti-tumor function. (FIG. 5A) Design of a conventional CD19-targeted CAR and a SMAD degrader-containing CD19 CAR. (FIG. 5B) Percent cytolysis of NALM6 tumor cells in a coculture assay with either CAR19 or CAR19+SMAD degrader T cells. CAR T cells and tumor cells were cocultured overnight at various ratios, with or without [conc] TGFb. (FIG. 5C) Normalized tumor area in an incucyte coculture assay. CAR19 or CAR19+SMAD degrader T cells were cultured at a 1:1 ratio with NALM6 tumor cells with or without exogenous TGFb for five days and imaged every hour. (FIG. 5D) Normalized T cell area in an incucyte coculture assay. (FIG. 5E) CAR T cell expansion in a long term proliferation coculture assay. CAR19 or CAR19+SMAD degrader T cells were cultured with CD19+ K562 tumor cells for three weeks, with weekly restimulation. (FIG. 5F) PD-1 abundance and % PD-1% of CD4+ CAR T cells.

FIG. 6A-6B—Targeted degradation of GFP with multiple E3 ligase recruiting domains. (FIG. 6A) Normalized GFP abundance in HEK293T cells dually transduced with GFP and a GFP degrader composed of the vhhGFP4 nanobody linked to the indicated ligase-binding domain. (FIG. 6B) Normalized GFP abundance in Jurkat cells dually transduced with GFP and a GFP degrader composed of the vhhGFP4 nanobody linked to the indicated ligase-binding domain.

FIG. 7—A ribbon model and domain diagram of Viral protein X (Vpx), an HIV derived protein.

FIG. 8—A strategy for reprogramming of Vpx to target GFP using an anti-GFP VHH single domain antibody. An anti-GFP VHH single domain antibody can be operably coupled to the Vpx protein.

FIG. 9—A schematic of an engineered Vpx protein capable of targeting an example target protein, GFP, showing two orientations for the Vpx-VHH engineered protein. The VHH single domain antibody can be fused or otherwise coupled to the N- and/or C-terminus of the Vpx.

FIG. 10—Results (as measured by a reduction in GFP fluorescence) from the engineered GFP-targeting Vpx proteins in HEK-293-nlsGFP cells described in connection with FIGS. 7-9.

FIG. 11—Schematic showing a CD19-activated SynNotch degradation system that includes a SynNotch receptor or degradation construct.

FIG. 12A-12B—GFP degradation in anti-CD19 synNotch activated cells.

FIG. 13A-13B—SMAD2/3 degradation in anti-CD19 synNotch activated cells.

The figures herein are for illustrative purposes and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlett, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2^{nd} ed., J. Wiley & Sons (New York, N.Y. 1994), March, Advanced Organic Chemistry Reactions, Mechanisms and Structure 4^{th} ed., John Wiley & Sons (New York, N.Y. 1992); and Marten H. Hofker and Jan van Deursen, Transgenic Mouse Methods and Protocols, 2^{nd} edition (2011).

Definitions of common terms and techniques in chemistry and organic chemistry can be found in Smith. Organic Synthesis, published by Academic Press. 2016; Tinoco et al. Physical Chemistry, 5^{th} edition (2013) published by Pearson; Brown et al., Chemistry, The Central Science 14^{th} ed. (2017), published by Pearson, Clayden et al., Organic Chemistry, 2^{nd} ed. 2012, published by Oxford University Press; Carey and Sunberg, Advanced Organic Chemistry, Part A: Structure and Mechanisms, 5^{th} ed. 2008, published by Springer; Carey and Sunberg, Advanced Organic Chemistry, Part B: Reactions and Synthesis, 5^{th} ed. 2010, published by Springer, and Vollhardt and Schore, Organic Chemistry, Structure and Function; 8^{th} ed. (2018) published by W. H. Freeman.

As used herein, the singular forms “a” “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g. a given confidence interval (e.g., 90%, 95%, or more confidence interval from the mean), such as variations of +/−10% or less, +/−5% or less, +/−1% or less, and +/−0.1% or less of and from the specified value, insofar such variations are appropriate to perform in the disclosed invention. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints. Where a range is expressed, a further aspect includes from the one particular value and/or to the other particular value. In some embodiments, where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less' and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, a “biological sample” refers to a sample obtained from, made by, secreted by, excreted by, or otherwise containing part of or from a biologic entity. A biologic sample can contain whole cells and/or live cells and/or cell debris, and/or cell products, and/or virus particles. The biological sample can contain (or be derived from) a “bodily fluid”. The biological sample can be obtained from an environment (e.g., water source, soil, air, and the like). Such samples are also referred to herein as environmental samples. As used herein “bodily fluid” refers to any non-solid excretion, secretion, or other fluid present in an organism and includes, without limitation unless otherwise specified or is apparent from the description herein, amniotic fluid, aqueous humor, vitreous humor, bile, blood or component thereof (e.g. plasma, serum, etc.), breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from an organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals (e.g., horses, pigs, cattle, sheep, goats, etc.) sport animals (dogs, horses, etc.), wild-animals (lions, bears, tigers, wolves, coyotes, deer, etc.), and/or pets (dogs, cats, rodents, guinea pigs, etc.). Subjects may also be non-mammalian animals, including, but not limited to, fish, crustaceans, reptiles, avians, etc. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment but may. Furthermore, the particular features, structures or characteristics may be combined in any suitable manner, as would be apparent to a person skilled in the art from this disclosure, in one or more embodiments. Moreover, as will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible. Furthermore, while some embodiments described herein include some, but not other features included in other embodiments, combinations of features of different embodiments are meant to be within the scope of the invention. For example, in the appended claims, any of the claimed embodiments can be used in any combination.

All publications and patents cited in this specification are cited to disclose and describe the methods and/or materials in connection with which the publications are cited. All publications, published patent documents, and patent applications cited herein are hereby incorporated by reference to the same extent as though each individual publication, published patent document, or patent application was specifically and individually indicated as being incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

### Overview

Targeted protein depletion is desirable for clinical and non-clinical applications. In some contexts, the targeted protein elimination or reduction has been achieved using genetic knockout or knockdown approaches. However, such approaches are still limited and inappropriate in some contexts. For example, such approaches are irreversible, poorly controllable, and/or difficult to scale to simultaneously target multiple genes/proteins in a single cell. Thus, there exists a need for improved strategies for targeted protein reduction.

With this in mind, described in exemplary embodiments herein are engineered bifunctional receptors that can be programmed to target a desired target protein, which can result in degradation of the target protein via an endogenous ubiquitination pathway. The engineered bifunctional receptors provide a modular platform in which target protein specificity can be changed by incorporating a different target binding domain and programming degradation by the ubiquitin pathway can be achieved by selection of E3 ligase binding domain. In some embodiments, the bifunctional receptor is composed of an E3 ligase binding domain and a target binding domain operatively coupled to the E3 ligase binding domain. In an example embodiment, the engineered bifunctional receptor inducibly binds an E3 ligase in response to a stimulus, such as a chemical, biological, or physical stimulus. In an example embodiment, the chemical stimulus is thalidomide or a derivative thereof. Induciblity can add an additional degree of control over activity of the engineered bifunctional receptor, and in turn, targeted protein degradation. In some embodiments, the engineered bifunctional receptor is capable of simultaneously targeting multiple different target proteins. The engineered bifunctional receptors can be expressed and/or delivered to a cell thereby resulting in targeted protein degradation within the cell. Such cells can be cell therapies, such as adoptive cell therapies. In some embodiments, the target protein(s) are those that mediate therapeutic cell dysfunction, exhaustion, and/or transformation. Other compositions, compounds, methods, features, uses, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Engineered Bifunctional Receptors

Generally, the engineered bifunctional receptors are polypeptides comprising an E3 ligase binding domain and a target binding domain operatively coupled to the E3 ligase binding domain. The engineered bifunctional receptors can facilitate induced proximity degradation of a target protein by bringing a target polypeptide bound to a target binding domain into proximity of a bound E3 ligase thereby facilitating ubiquitination of the target polypeptide and subsequent degradation by the ubiquitin pathway. The engineered bifunctional receptors can be programmable for any desired target protein for which a binding domain can be identified or generated. The engineered bifunctional receptors can be configured to respond to a stimulus, thus allowing targeted degradation to be further tuned. Further control over activity of the bifunctional receptors can be achieved at the genetic level by controlling expression of one or more components of the bifunctional receptors. The engineered bifunctional receptors can be encoded by polynucleotides, which can optionally be included in vector(s) or vector system(s). The polynucleotides, vectors, proteins, or combinations thereof can be delivered to a cell using any suitable method or technique. The engineered bifunctional receptors, polynucleotides, vectors, delivery vehicles are now described in greater detail.

### Engineered Bifunctional Receptors

Described in several example embodiments herein are engineered bifunctional receptors that comprise an E3 ligase binding domain and a target binding domain operatively coupled to the E3 ligase binding domain. In some embodiments, the target binding domain is operatively linked to the E3 ligase binding domain directly (e.g., by fusion) or indirectly (e.g., via a linker). In some embodiments, the target binding domain is operatively linked to the E3 ligase binding domain via a flexible linker.

In some embodiments the engineered bifunctional receptor inducibly binds an E3 ligase in response to a stimulus. In some embodiments, the engineered bifunctional receptor further comprises a stimulus responsive domain.

In some embodiments, the engineered bifunctional receptor binds two or more different target polypeptides. Without being bound by theory, the configuration of some embodiments of the engineered bifunctional receptor allows for multiple different target proteins to be targeted by the engineered bifunctional receptor.

### Domains

### E3 Ligase Binding Domain

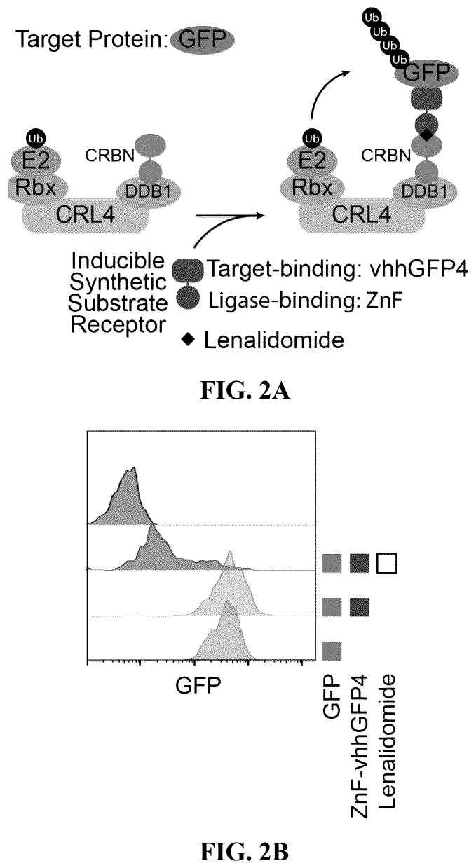

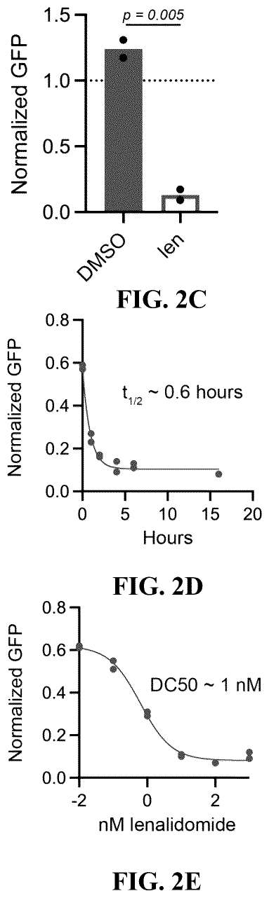

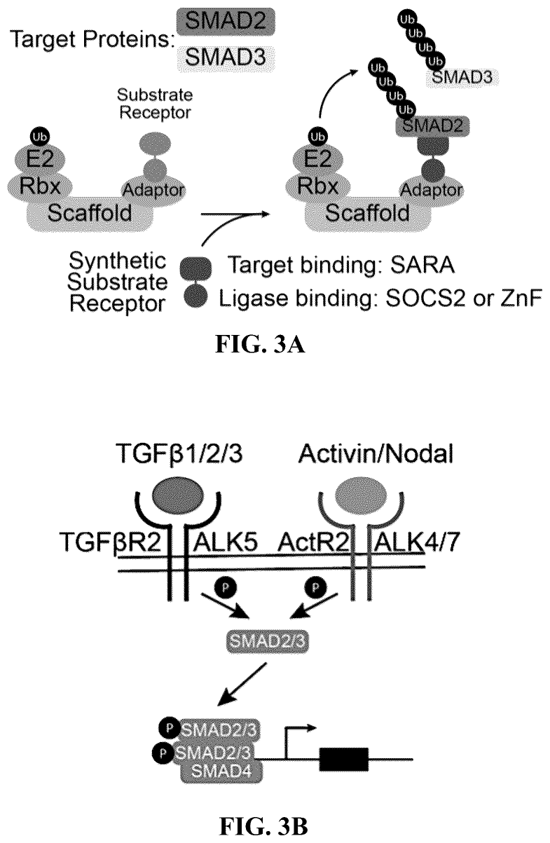

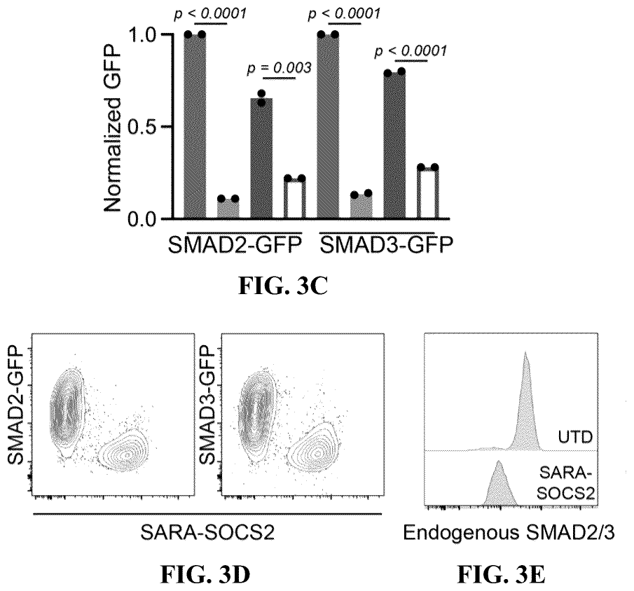

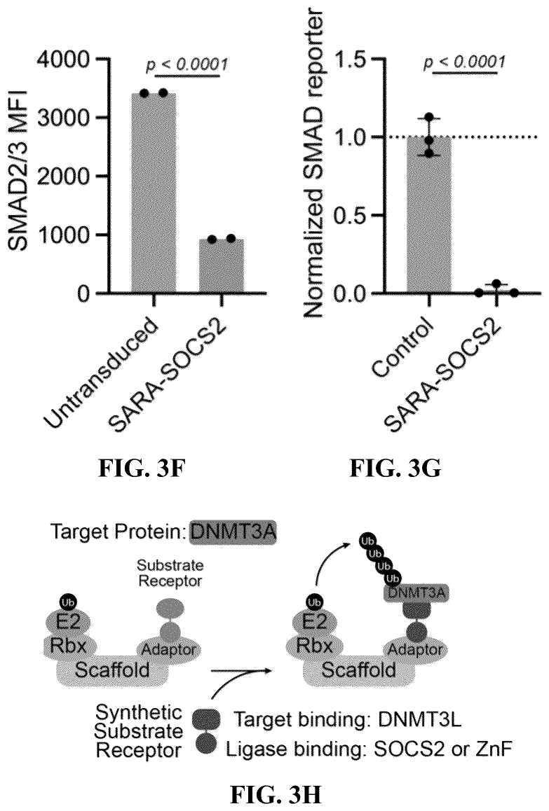

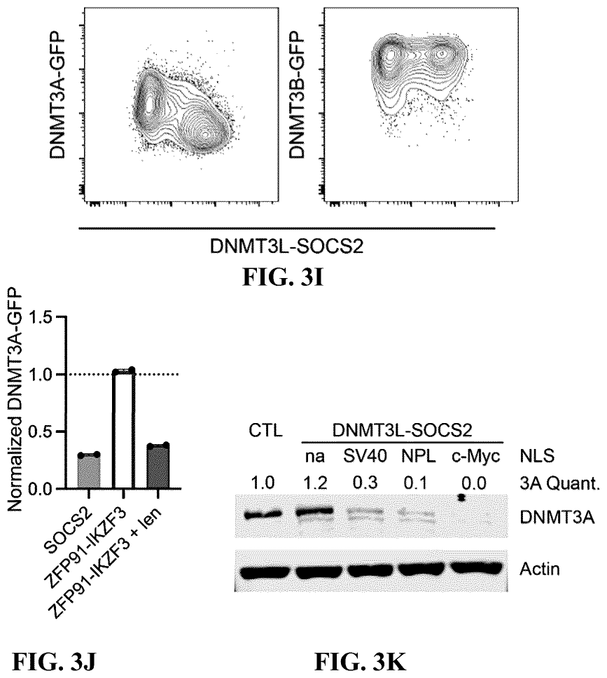

E3 ubiquitin ligases (“E3 ligases”) are a large family of enzymes that join in a three-enzyme ubiquitination cascade together with ubiquitin activating enzyme E1 and ubiquitin conjugating enzyme E2. E3 ligases play an essential role in catalyzing the ubiquitination process and transferring ubiquitin protein to attach the lysine site of targeted substrates. See e.g., Yang et al., Mol. Biomed. 2021. 2(23). The engineered bifunctional receptor of the present invention comprises one or more E3 ligase binding domains, which without being bound by theory, can recruit and/or bind an E3 ligase. By binding of an E3 ligase and a target polypeptide the engineered bifunctional receptor can facilitate induced proximity degradation of the target polypeptide via the ubiquitination pathway (see e.g., Yang et al., Mol. Biomed. 2021. 2(23), particularly at FIGS. 2 and 3). In general, the E3 ligase binding domain can specifically bind an E3 ligase or portion thereof. As used herein, the term “specific binding” refers to non-covalent physical association of a first and a second moiety wherein the association between the first and second moieties is at least 2 times as strong, at least 5 times as strong as, at least 10 times as strong as, at least 50 times as strong as, at least 100 times as strong as, or stronger than the association of either moiety with most or all other moieties present in the environment in which binding occurs. Binding of two or more entities may be considered specific if the equilibrium dissociation constant, Kd, is 10⁻³ M or less, 10⁻⁴ M or less, 10⁻⁵ M or less, 10⁻⁶ M or less, 10⁻⁷ M or less, 10⁻⁸ M or less, 10⁻⁹ M or less, 10⁻¹⁰ M or less, 10⁻¹¹ M or less, or 10⁻¹² M or less under the conditions employed, e.g., under physiological conditions such as those inside a cell or consistent with cell survival. In some embodiments, specific binding can be accomplished by a plurality of weaker interactions (e.g., a plurality of individual interactions, wherein each individual interaction is characterized by a Kd of greater than 10⁻³ M). In some embodiments, specific binding, which can be referred to as “molecular recognition,” is a saturable binding interaction between two entities that is dependent on complementary orientation of functional groups on each entity. Examples of specific binding interactions include primer-polynucleotide interaction, aptamer-aptamer target interactions, antibody-antigen interactions, avidin-biotin interactions, ligand-receptor interactions, metal-chelate interactions, hybridization between complementary nucleic acids, etc.

In some embodiments, the E3 ligase binding domain is a HECT E3 ligase binding domain. In some embodiments, the E3 ligase binding domain is a RING E3 ligase binding domain. In some embodiments, the E3 ligase binding domain is a U-box E3 ligase binding domain. In some embodiments, the E3 ligase binding domain is an RBR E3 ligase binding domain. In some embodiments, the E3 ligase binding domain is capable of specifically binding an E3 ligase as part of the database of human E3 ligases available at https.//esbl.nhlbi.nih.gov/Databases/KSBP2/Targets/Lists/E3-ligases/. See also Medvar et al., Physiol Genomics. 2016 Jul. 1; 48(7): 502-512. In some embodiments the E3 ligase binding domain is capable of specifically binding an E3 ligase as set forth in Table 1 or a homologue or orthologue thereof. Specific binding of a E3 ligase binding domain by an E3 ligase can be determined using any suitable method or technique generally known in the art.

TABLE 1
Exemplary E3 ligases

| Gene Symbol | Protein Name | Domain | RefSeq | Swiss-Prot |
|---|---|---|---|---|
| AFF4 | AF4/FMR2 family member 4 | UBOX | 7656879 | Q9UHB7 |
| AMFR | autocrine motility factor receptor, E3 ubiquitin protein ligase | RING | 21071001 | Q9UKV5 |
| ANAPC11 | anaphase-promoting complex subunit 11 isoform 2 | RING | 574957206 | Q9NYG5 |
| ANKIB1 | ankyrin repeat and IBR domain-containing protein 1 | RING | 148596953 | Q9P2G1 |
| AREL1 | apoptosis resistant E3 ubiquitin protein ligase 1 | HECTc | 87116668 | O15033 |
| ARIH1 | ariadne RBR E3 ubiquitin protein ligase 1 | RING | 187761373 | Q9Y4X5 |
| ARIH2 | ariadne RBR E3 ubiquitin protein ligase 2 | RING | 5453557 | O95376 |
| BARD1 | BRCA1 associated RING domain 1 | RING | 543583789 | Q99728 |
| BFAR | bifunctional apoptosis regulator | RING | 7706091 | Q9NZS9 |
| BIRC2 | baculoviral IAP repeat containing 2 | RING | 390608639 | Q13490 |
| BIRC3 | baculoviral IAP repeat containing 3 | RING | 33946285 | Q13489 |
| BIRC7 | baculoviral IAP repeat containing 7 | RING | 21536421 | Q96CA5 |
| BIRC8 | baculoviral IAP repeat-containing protein 8 | RING | 44680139 | Q96P09 |
| BMI1 | BMI1 polycomb ring finger proto-oncogene* | RING | 27883842 | P35226 |
| BRAP | BRCA1 associated protein | RING | 188497705 | Q7Z569 |
| BRCA1 | breast cancer 1, early onset* | RING | 237681125 | P38398 |
| CBL | Cbl proto-oncogene, E3 ubiquitin protein ligase | RING | 52426745 | P22681 |
| CBLB | Cbl proto-oncogene B, E3 ubiquitin protein ligase | RING | 54112420 | Q13191 |
| CBLC | Cbl proto-oncogene C, E3 ubiquitin protein ligase | RING | 195927030 | Q9ULV8 |
| CBLL1 | Cbl proto-oncogene-like 1, E3 ubiquitin protein ligase | RING | 546230945 | Q75N03 |
| CCDC36 | coiled-coil domain-containing protein 36 | RING | 206597549 | Q8IYA8 |
| CCNB1IP1 | cyclin B1 interacting protein 1, E3 ubiquitin protein ligase | RING | 116812640 | Q9NPC3 |
| CGRRF1 | cell growth regulator with RING finger domain protein 1 | RING | 5729765 | Q99675 |
| CHFR | checkpoint with forkhead and ring finger domains, E3 ubiquitin | RING | 239048909 | Q96EP1 |
| | protein ligase | | | |
| CNOT4 | CCR4-NOT transcription complex, subunit 4 | RING | 300069017 | O95628 |
| CUL9 | cullin-9 | RING | 24307991 | Q8IWT3 |
| CYHR1 | cysteine and histidine-rich protein 1 isoform 2 precursor | RING | 193788637 | Q6ZMK1 |
| DCST1 | DC-STAMP domain-containing protein 1 isoform 2 | RING | 219521926 | Q5T197 |
| DTX1 | deltex 1, E3 ubiquitin ligase | RING | 41352718 | Q86Y01 |
| DTX2 | deltex 2, E3 ubiquitin ligase | RING | 156564386 | Q86UW9 |
| DTX3 | deltex 3, E3 ubiquitin ligase | RING | 555943736 | Q8N919 |
| DTX3L | deltex 3 like, E3 ubiquitin ligase | RING | 19923717 | Q8TDB6 |
| DTX4 | deltex 4, E3 ubiquitin ligase | RING | 663071089 | Q9Y2E6 |
| DZIP3 | DAZ interacting zinc finger protein 3 | RING | 7662244 | Q86Y13 |
| E4F1 | E4F transcription factor 1 | zf-C2H2 | 571026678 | Q66K89 |
| FANCL | Fanconi anemia, complementation group L | RING | 167860138 | Q9NW38 |
| G2E3 | G2/M-phase specific E3 ubiquitin protein ligase | HECTc | 809279653 | Q7L622 |
| HACE1 | HECT domain and ankyrin repeat containing E3 ubiquitin protein | HECTc | 116875852 | Q8IYU2 |
| | ligase 1 | | | |
| HECTD1 | HECT domain containing E3 ubiquitin protein ligase 1 | HECTc | 612339310 | Q9ULT8 |
| HECTD2 | HECT domain containing E3 ubiquitin protein ligase 2 | HECTc | 546231434 | Q5U5R9 |
| HECTD3 | HECT domain containing E3 ubiquitin protein ligase 3 | HECT | 157738609 | Q5T447 |
| HECTD4 | HECT domain containing E3 ubiquitin protein ligase 4 | HECTc | 359718912 | Q9Y4D8 |
| HECW1 | HECT, C2 and WW domain containing E3 ubiquitin protein ligase | HECTc | 559098413 | Q76N89 |
| | 1 | | | |
| HECW2 | HECT, C2 and WW domain containing E3 ubiquitin protein ligase | HECTc | 756140743 | Q9P2P5 |
| | 2 | | | |
| HERC1 | HECT and RLD domain containing E3 ubiquitin protein ligase | HECTc | 126131099 | Q15751 |
| | family member 1 | | | |
| HERC2 | HECT and RLD domain containing E3 ubiquitin protein ligase 2* | HECTc | 126032348 | O95714 |
| HERC3 | HECT and RLD domain containing E3 ubiquitin protein ligase 3 | HECTc | 410110896 | Q15034 |
| HERC4 | HECT and RLD domain containing E3 ubiquitin protein ligase 4 | HECTc | 499137513 | Q5GLZ8 |
| HERC5 | HECT and RLD domain containing E3 ubiquitin protein ligase 5 | HECTc | 110825982 | Q9UII4 |
| HERC6 | HECT and RLD domain containing E3 ubiquitin protein ligase | HECTc | 259089405 | Q8IVU3 |
| | family member 6 | | | |
| HLTF | helicase-like transcription factor | RING | 21071054 | Q14527 |
| HUWE1 | HECT, UBA and WWE domain containing 1, E3 ubiquitin protein | HECTc | 61676188 | Q7Z6Z7 |
| | ligase | | | |
| IRF2BP1 | interferon regulatory factor 2 binding protein 1 | RING | 24308115 | Q8IU81 |
| IRF2BP2 | interferon regulatory factor 2-binding protein 2 isoform B | RING | 116734706 | Q7Z5L9 |
| IRF2BPL | interferon regulatory factor 2-binding protein-like | RING | 33457336 | Q9H1B7 |
| Itch | itchy E3 ubiquitin protein ligase | HECTc | 380420338 | Q96J02 |
| KCMF1 | potassium channel modulatory factor 1 | RING | 46852178 | Q9P0J7 |
| KMT2C | histone-lysine N-methyltransferase 2C | RING | 91718902 | Q8NEZ4 |
| KMT2D | histone-lysine N-methyltransferase 2D | RING | 148762969 | O14686 |
| LNX1 | ligand of numb-protein X 1, E3 ubiquitin protein ligase | RING | 187607085 | Q8TBB1 |
| LNX2 | ligand of numb-protein X 2 | RING | 24025688 | Q8N448 |
| LONRF1 | LON peptidase N-terminal domain and RING finger protein 1 | RING | 87080813 | Q17RB8 |
| LONRF2 | LON peptidase N-terminal domain and RING finger protein 2 | RING | 148528975 | Q1L5Z9 |
| LONRF3 | LON peptidase N-terminal domain and RING finger protein 3 | RING | 574280908 | Q496Y0 |
| | isoform 3 | | | |
| LRSAM1 | leucine rich repeat and sterile alpha motif containing 1 | RING | 299758423 | Q6UWE0 |
| LTN1 | listerin E3 ubiquitin protein ligase 1 | RING | 231573214 | O94822 |
| MAEA | macrophage erythroblast attacher isoform 4 | RING | 661860329 | Q7L5Y9 |
| MAP3K1 | mitogen-activated protein kinase kinase kinase 1 | RING | 153945765 | Q13233 |
| MARCH1 | membrane-associated ring finger (C3HC4) 1, E3 ubiquitin protein | RING | 261823980 | Q8TCQ1 |
| | ligase | | | |
| MARCH10 | membrane-associated ring finger (C3HC4) 10, E3 ubiquitin | RING | 571026626 | Q8NA82 |
| | protein ligase | | | |
| MARCH11 | membrane-associated ring finger (C3HC4) 11 | RING | 156523244 | A6NNE9 |
| MARCH2 | membrane-associated ring finger (C3HC4) 2, E3 ubiquitin protein | RING | 53729334 | Q9P0N8 |
| | ligase | | | |
| MARCH3 | membrane-associated ring finger (C3HC4) 3, E3 ubiquitin protein | RING | 30425370 | Q86UD3 |
| | ligase | | | |
| MARCH4 | membrane-associated ring finger (C3HC4) 4, E3 ubiquitin protein | RING | 57977325 | Q9P2E8 |
| | ligase | | | |
| MARCH5 | membrane-associated ring finger (C3HC4) 5 | RING | 8923415 | Q9NX47 |
| MARCH6 | membrane-associated ring finger (C3HC4) 6, E3 ubiquitin protein | RING | 397787554 | O60337 |
| | ligase | | | |
| MARCH7 | membrane-associated ring finger (C3HC4) 7, E3 ubiquitin protein | RING | 544346245 | Q9H992 |
| | ligase | | | |
| MARCH8 | membrane-associated ring finger (C3HC4) 8, E3 ubiquitin protein | RING | 544583444 | Q5T0T0 |
| | ligase | | | |
| MARCH9 | membrane-associated ring finger (C3HC4) 9 | RING | 40255016 | Q86YJ5 |
| Mdm2 | MDM2 oncogene, E3 ubiquitin protein ligase | RING | 510937018 | Q00987 |
| MDM4 | protein Mdm4 isoform 7 | RING | 512125239 | O15151 |
| MECOM | MDS1 and EVI1 complex locus protein EVI1 isoform b | RING | 327180722 | Q03112 |
| MEX3A | RNA-binding protein MEX3A | RING | 147902746 | A1L020 |
| MEX3B | RNA-binding protein MEX3B | RING | 47716512 | Q6ZN04 |
| MEX3C | mex-3 RNA binding family member C | RING | 148229134 | Q5U5Q3 |
| MEX3D | RNA-binding protein MEX3D isoform 2 | RING | 291621664 | Q86XN8 |
| MGRN1 | mahogunin ring finger 1, E3 ubiquitin protein ligase | RING | 334883182 | O60291 |
| MIB1 | mindbomb E3 ubiquitin protein ligase 1 | RING | 30348954 | Q86YT6 |
| MIB2 | mindbomb E3 ubiquitin protein ligase 2 | RING | 282394038 | Q96AX9 |
| MID1 | midline 1 | RING | 300797215 | O15344 |
| MID2 | midline 2 | RING | 223890259 | Q9UJV3 |
| MKRN1 | makorin ring finger protein 1 | RING | 619329024 | Q9UHC7 |
| MKRN2 | makorin ring finger protein 2 | RING | 411147393 | Q9H000 |
| MKRN3 | makorin ring finger protein 3 | RING | 5032243 | Q13064 |
| MKRN4P | Putative E3 ubiquitin-protein ligase makorin-4 | RING | 17368441 | Q13434 |
| MNAT1 | CDK-activating kinase assembly factor MAT1 isoform 2 | RING | 295789085 | P51948 |
| MSL2 | male-specific lethal 2 homolog (Drosophila) | RING | 224028284 | Q9HC17 |
| MUL1 | mitochondrial E3 ubiquitin protein ligase 1* | RING | 171542821 | Q969V5 |
| MYCBP2 | MYC binding protein 2, E3 ubiquitin protein ligase | RING | 291190787 | O75592 |
| MYLIP | myosin regulatory light chain interacting protein | RING | 38788243 | Q8WY64 |
| NEDD4 | neural precursor cell expressed, developmentally down-regulated | HECTc | 547235493 | P46934 |
| | 4, E3 ubiquitin protein ligase | | | |
| NEDD4L | neural precursor cell expressed, developmentally down-regulated | HECTc | 345478677 | Q96PU5 |
| | 4-like, E3 ubiquitin protein ligase | | | |
| NEURL1 | neuralized E3 ubiquitin protein ligase 1 | RING | 223278368 | O76050 |
| NEURL1B | neuralized E3 ubiquitin protein ligase 1B | RING | 815890887 | A8MQ27 |
| NEURL3 | neuralized E3 ubiquitin protein ligase 3 | RING | 550822415 | Q96EH8 |
| NFX1 | transcriptional repressor NF-X1 isoform 3 | RING | 22212927 | Q12986 |
| NFXL1 | NF-X1-type zinc finger protein NFXL1 | RING | 520261838 | Q6ZNB6 |
| NHLRC1 | NHL repeat containing E3 ubiquitin protein ligase 1 | RING | 40255283 | Q6VVB1 |
| NOSIP | nitric oxide synthase-interacting protein | UBOX | 401709927 | Q9Y314 |
| NSMCE1 | non-SMC element 1 homolog (S. cerevisiae)* | RING | 94721324 | Q8WV22 |
| PARK2 | parkin RBR E3 ubiquitin protein ligase | RING | 169790973 | O60260 |
| PCGF1 | polycomb group ring finger 1 | RING | 109240538 | Q9BSM1 |
| PCGF2 | polycomb group ring finger 2* | RING | 6005964 | P35227 |
| PCGF3 | polycomb group RING finger protein 3 | RING | 31742478 | Q3KNV8 |
| PCGF5 | polycomb group RING finger protein 5 | RING | 380036033 | Q86SE9 |
| PCGF6 | polycomb group RING finger protein 6 isoform a | RING | 58761530 | Q9BYE7 |
| PDZRN3 | PDZ domain containing ring finger 3 | RING | 735367623 | Q9UPQ7 |
| PDZRN4 | PDZ domain containing ring finger 4 | RING | 257196172 | Q6ZMN7 |
| PELI1 | pellino E3 ubiquitin protein ligase 1 | PELI | 11037063 | Q96FA3 |
| PELI2 | pellino E3 ubiquitin protein ligase family member 2 | PELI | 10864063 | Q9HAT8 |
| PELI3 | pellino E3 ubiquitin protein ligase family member 3 | PELI | 341823689 | Q8N2H9 |
| PEX10 | peroxisome biogenesis factor 10 isoform 1 | RING | 24797089 | O60683 |
| PEX12 | peroxisome assembly protein 12 | RING | 4505721 | O00623 |
| PEX2 | peroxisome biogenesis factor 2 | RING | 289063469 | P28328 |
| PHF7 | PHD finger protein 7 isoform 2 | RING | 506325002 | Q9BWX1 |
| PHRF1 | PHD and RING finger domain-containing protein 1 isoform 1 | RING | 557440888 | Q9P1Y6 |
| PJA1 | praja ring finger 1, E3 ubiquitin protein ligase | RING | 530421802 | Q8NG27 |
| PJA2 | praja ring finger 2, E3 ubiquitin protein ligase | RING | 157412255 | O43164 |
| PLAG1 | zinc finger protein PLAG1 isoform b | RING | 167857798 | Q6DJT9 |
| PLAGL1 | zinc finger protein PLAGL1 isoform 1 | RING | 573459768 | Q9UM63 |
| PML | promyelocytic leukemia* | RING | 109637788 | P29590 |
| PPIL2 | peptidylprolyl isomerase (cyclophilin)-like 2 | UBOX | 22547215 | Q13356 |
| PRPF19 | pre-mRNA processing factor 19 | UBOX | 7657381 | Q9UMS4 |
| RAD18 | RAD18 homolog (S. cerevisiae) | RING | 256818821 | Q9NS91 |
| RAG1 | recombination activating gene 1 | RING | 4557841 | P15918 |
| RAPSN | 43 kDa receptor-associated protein of the synapse isoform 2 | RING | 38045928 | Q13702 |
| RBBP6 | retinoblastoma binding protein 6 | RING | 33620769 | Q7Z6E9 |
| RBCK1 | RanBP-type and C3HC4-type zinc finger containing 1 | RING | 144953898 | Q9BYM8 |
| RBX1 | ring-box 1, E3 ubiquitin protein ligase** | RING | 7657508 | P62877 |
| RC3H1 | ring finger and CCCH-type domains 1 | RING | 664806085 | Q5TC82 |
| RC3H2 | ring finger and CCCH-type domains 2 | RING | 156105699 | Q9HBD1 |
| RCHY1 | ring finger and CHY zinc finger domain containing 1, E3 | RING | 512749780 | Q96PM5 |
| | ubiquitin protein ligase | | | |
| RFFL | ring finger and FYVE-like domain containing E3 ubiquitin protein | RING | 62865649 | Q8WZ73 |
| | ligase | | | |
| RFPL1 | ret finger protein-like 1 | RING | 149408130 | O75677 |
| RFPL2 | ret finger protein-like 2 isoform 3 | RING | 227116308 | O75678 |
| RFPL3 | ret finger protein-like 3 isoform 1 | RING | 149408135 | O75679 |
| RFPL4A | ret finger protein-like 4A | RING | 222446629 | A6NLU0 |
| RFPL4AL1 | ret finger protein-like 4A-like protein 1 | RING | 475807831 | F8VTS6 |
| RFPL4B | ret finger protein-like 4B | RING | 153791360 | Q6ZWI9 |
| RFWD2 | ring finger and WD repeat domain 2, E3 ubiquitin protein ligase | RING | 557786192 | Q8NHY2 |
| RFWD3 | ring finger and WD repeat domain 3 | RING | 71143112 | Q6PCD5 |
| RING1 | ring finger protein 1* | RING | 51479192 | Q06587 |
| RLF | zinc finger protein Rlf | RING | 157671949 | Q13129 |
| RLIM | ring finger protein, LIM domain interacting | RING | 34452686 | Q9NVW2 |
| RMND5A | protein RMD5 homolog A | RING | 12232469 | Q9H871 |
| RMND5B | protein RMD5 homolog B isoform a | RING | 571031354 | Q96G75 |
| RNF10 | ring finger protein 10 | RING | 34452681 | Q8N5U6 |
| RNF103 | ring finger protein 103 | RING | 312147329 | O00237 |
| RNF11 | ring finger protein 11 | RING | 7657520 | Q9Y3C5 |
| RNF111 | ring finger protein 111* | RING | 395455085 | Q6ZNA4 |
| RNF112 | RING finger protein 112 | RING | 264681488 | Q9ULX5 |
| RNF113A | RING finger protein 113A | RING | 5902158 | O15541 |
| RNF113B | RING finger protein 113B | RING | 30578416 | Q8IZP6 |
| RNF114 | ring finger protein 114 | RING | 8923898 | Q9Y508 |
| RNF115 | ring finger protein 115 | RING | 530363886 | Q9Y4L5 |
| RNF121 | RING finger protein 121 isoform b | RING | 665506028 | Q9H920 |
| RNF122 | RING finger protein 122 | RING | 38045931 | Q9H9V4 |
| RNF123 | ring finger protein 123 | RING | 37588869 | Q5XPI4 |
| RNF125 | ring finger protein 125, E3 ubiquitin protein ligase | RING | 37595555 | Q96EQ8 |
| RNF126 | ring finger protein 126 | RING | 37622894 | Q9BV68 |
| RNF128 | ring finger protein 128, E3 ubiquitin protein ligase | RING | 37588873 | Q8TEB7 |
| RNF13 | ring finger protein 13 | RING | 34577087 | O34567 |
| RNF130 | ring finger protein 130 | RING | 526253083 | Q86XS8 |
| RNF133 | ring finger protein 133 | RING | 21040269 | Q8WVZ7 |
| RNF135 | ring finger protein 135 | RING | 297139721 | Q8IUD6 |
| RNF138 | ring finger protein 138, E3 ubiquitin protein ligase | RING | 395455059 | Q8WVD3 |
| RNF139 | ring finger protein 139 | RING | 21314654 | Q8WU17 |
| RNF14 | ring finger protein 14 | RING | 318067966 | Q9UBS8 |
| RNF141 | ring finger protein 141 | RING | 21361493 | Q8WVD5 |
| RNF144A | ring finger protein 144A | RING | 38045938 | P50876 |
| RNF144B | ring finger protein 144B | RING | 50284696 | Q7Z419 |
| RNF145 | RING finger protein 145 isoform 5 | RING | 313661397 | Q96MT1 |
| RNF146 | ring finger protein 146 | RING | 338827727 | Q9NTX7 |
| RNF148 | RING finger protein 148 precursor | RING | 37675277 | Q8N7C7 |
| RNF149 | ring finger protein 149 | RING | 284447287 | Q8NC42 |
| RNF150 | RING finger protein 150 precursor | RING | 58331204 | Q9ULK6 |
| RNF151 | ring finger protein 151 | RING | 87241872 | Q2KHN1 |
| RNF152 | ring finger protein 152 | RING | 27734873 | Q8N8N0 |
| RNF157 | RING finger protein 157 | RING | 58743365 | Q96PX1 |
| RNF165 | RING finger protein 165 isoform 1 | RING | 57165361 | Q6ZSG1 |
| RNF166 | RING finger protein 166 isoform 2 | RING | 284795357 | Q96A37 |
| RNF167 | ring finger protein 167 | RING | 14149702 | Q9H6Y7 |
| RNF168 | ring finger protein 168, E3 ubiquitin protein ligase* | RING | 31377566 | Q8IYW5 |
| RNF169 | ring finger protein 169 | RING | 148839382 | Q8NCN4 |
| RNF17 | RING finger protein 17 isoform 2 | RING | 297139728 | Q9BXT8 |
| RNF170 | ring finger protein 170 | RING | 237858658 | Q96K19 |
| RNF175 | RING finger protein 175 | RING | 27734859 | Q8N4F7 |
| RNF180 | ring finger protein 180 | RING | 165932346 | Q86T96 |
| RNF181 | ring finger protein 181 | RING | 7706039 | Q9P0P0 |
| RNF182 | ring finger protein 182 | RING | 259013545 | Q8N6D2 |
| RNF183 | RING finger protein 183 | RING | 153252110 | Q96D59 |
| RNF185 | ring finger protein 185 | RING | 209529683 | Q96GF1 |
| RNF186 | RING finger protein 186 | RING | 9506663 | Q9NXI6 |
| RNF187 | ring finger protein 187 | RING | 256225838 | Q5TA31 |
| RNF19A | ring finger protein 19A, RBR E3 ubiquitin protein ligase | RING | 525459427 | Q9NV58 |
| RNF19B | ring finger protein 19B | RING | 664806060 | Q6ZMZ0 |
| RNF2 | ring finger protein 2* | RING | 6005747 | Q99496 |
| RNF20 | ring finger protein 20, E3 ubiquitin protein ligase | RING | 34878777 | Q5VTR2 |
| RNF207 | RING finger protein 207 | RING | 124487387 | Q6ZRF8 |
| RNF208 | ring finger protein 208 | RING | 119220603 | Q9H0X6 |
| RNF212 | probable E3 SUMO-protein ligase RNF212 isoform c* | RING | 300863140 | Q495C1 |
| RNF212B | RING finger protein 212B | RING | 536291168 | A8MTL3 |
| RNF213 | ring finger protein 213 | RING | 597709777 | Q63HN8 |
| RNF214 | RING finger protein 214 isoform 2 | RING | 503775288 | Q8ND24 |
| RNF215 | RING finger protein 215 precursor | RING | 63025220 | Q9Y6U7 |
| RNF216 | ring finger protein 216 | RING | 46370057 | Q9NWF9 |
| RNF217 | ring finger protein 217 | RING | 556562062 | Q8TC41 |
| RNF219 | RING finger protein 219 | RING | 88759348 | Q5W0B1 |
| RNF220 | ring finger protein 220 | RING | 46397375 | Q5VTB9 |
| RNF222 | PREDICTED: RING finger protein 222 isoform X1 | RING | 767992469 | A6NCQ9 |
| RNF223 | RING finger protein 223 | RING | 327412333 | E7ERA6 |
| RNF224 | PREDICTED: RING finger protein 224 isoform X1 | RING | 767958041 | P0DH78 |
| RNF225 | PREDICTED: RING finger protein 225 isoform X1 | RING | 578834770 | M0QZC1 |
| RNF24 | RING finger protein 24 isoform 2 | RING | 197245452 | Q9Y225 |
| RNF25 | ring finger protein 25 | RING | 34878787 | Q96BH1 |
| RNF26 | RING finger protein 26 | RING | 14042925 | Q9BY78 |
| RNF31 | ring finger protein 31 | RING | 109150431 | Q96EP0 |
| RNF32 | RING finger protein 32 isoform c | RING | 815729259 | Q9H0A6 |
| RNF34 | ring finger protein 34, E3 ubiquitin protein ligase | RING | 378925642 | Q969K3 |
| RNF38 | ring finger protein 38 | RING | 37577185 | Q9H0F5 |
| RNF39 | RING finger protein 39 isoform 1 | RING | 297206763 | Q9H2S5 |
| RNF4 | ring finger protein 4* | RING | 297139779 | P78317 |
| RNF40 | ring finger protein 40, E3 ubiquitin protein ligase | RING | 557440901 | O75150 |
| RNF41 | ring finger protein 41, E3 ubiquitin protein ligase | RING | 338827618 | Q9H4P4 |
| RNF43 | ring finger protein 43 | RING | 770075668 | Q68DV7 |
| RNF44 | RING finger protein 44 | RING | 7662486 | Q7L0R7 |
| RNF5 | ring finger protein 5, E3 ubiquitin protein ligase | RING | 5902054 | Q99942 |
| RNF6 | ring finger protein (C3H2C3 type) 6 | RING | 34305295 | Q9Y252 |
| RNF7 | ring finger protein 7** | RING | 319004144 | Q9UBF6 |
| RNF8 | ring finger protein 8, E3 ubiquitin protein ligase | RING | 34304336 | O76064 |
| RNFT1 | RING finger and transmembrane domain-containing protein 1 | RING | 109134327 | Q5M7Z0 |
| RNFT2 | RING finger and transmembrane domain-containing protein 2 | RING | 158186614 | Q96EX2 |
| | isoform 1 | | | |
| RSPRY1 | RING finger and SPRY domain-containing protein 1 isoform 1 | RING | 762205506 | Q96DX4 |
| | precursor | | | |
| SCAF11 | protein SCAF11 | RING | 117676384 | Q99590 |
| SH3RF1 | SH3 domain containing ring finger 1 | RING | 51988887 | Q7Z6J0 |
| SH3RF2 | SH3 domain containing ring finger 2 | RING | 222446607 | Q8TEC5 |
| SH3RF3 | SH3 domain-containing RING finger protein 3 precursor | RING | 150010572 | Q8TEJ3 |
| SHPRH | SNF2 histone linker PHD RING helicase, E3 ubiquitin protein | RING | 289547541 | Q149N8 |
| | ligase | | | |
| SIAH1 | siah E3 ubiquitin protein ligase 1 | RING | 63148618 | Q8IUQ4 |
| SIAH2 | siah E3 ubiquitin protein ligase 2 | RING | 31982899 | O43255 |
| SIAH3 | siah E3 ubiquitin protein ligase family member 3 | RING | 110578665 | Q8IW03 |
| SMURF1 | SMAD specific E3 ubiquitin protein ligase 1 | HECTc | 63148618 | Q9HCE7 |
| SMURF2 | SMAD specific E3 ubiquitin protein ligase 2 | HECTc | 12232397 | Q9HAU4 |
| STUB1 | STIP1 homology and U-box containing protein 1, E3 ubiquitin | UBOX | 645912990 | Q9UNE7 |
| | protein ligase | | | |
| SYVN1 | synovial apoptosis inhibitor 1, synoviolin | RING | 27436927 | Q86TM6 |
| TMEM129 | E3 ubiquitin-protein ligase TM129 isoform a | RING | 187936961 | A0AVI4 |
| Topors | topoisomerase I binding, arginine/serine-rich, E3 ubiquitin protein | RING | 307344673 | Q9NS56 |
| | ligase* | | | |
| TRAF2 | TNF receptor-associated factor 2 | RING | 22027612 | Q12933 |
| TRAF3 | TNF receptor-associated factor 3 | RING | 313661483 | Q13114 |
| TRAF4 | TNF receptor-associated factor 4 | RING | 22027622 | Q9BUZ4 |
| TRAF5 | TNF receptor-associated factor 5 | RING | 77404348 | O00463 |
| TRAF6 | TNF receptor-associated factor 6, E3 ubiquitin protein ligase | RING | 22027630 | Q9Y4K3 |
| TRAF7 | TNF receptor-associated factor 7, E3 ubiquitin protein ligase* | RING | 45594312 | Q6Q0C0 |
| TRAIP | TRAF interacting protein | RING | 40807469 | Q9BWF2 |
| TRIM10 | tripartite motif-containing protein 10 isoform 1 | RING | 157426898 | Q9UDY6 |
| TRIM11 | tripartite motif containing 11 | RING | 21630277 | Q96F44 |
| TRIM13 | tripartite motif containing 13 | RING | 55953112 | O60858 |
| TRIM15 | tripartite motif-containing protein 15 | RING | 149193331 | Q9C019 |
| TRIM17 | tripartite motif containing 17 | RING | 198442878 | Q9Y577 |
| TRIM2 | tripartite motif containing 2 | RING | 705044345 | Q9C040 |
| TRIM21 | tripartite motif containing 21 | RING | 15208660 | P19474 |
| TRIM22 | tripartite motif containing 22 | RING | 313760629 | Q8IYM9 |
| TRIM23 | tripartite motif containing 23 | RING | 15208643 | P36406 |
| TRIM24 | tripartite motif containing 24 | RING | 47419911 | O15164 |
| TRIM25 | tripartite motif containing 25 | RING | 68160937 | Q14258 |
| TRIM26 | tripartite motif-containing protein 26 | RING | 338753391 | Q12899 |
| TRIM27 | tripartite motif containing 27 | RING | 5730009 | P14373 |
| TRIM28 | tripartite motif containing 28* | RING | 5032179 | Q123263 |
| TRIM3 | tripartite motif-containing protein 3 isoform 2 | RING | 351721595 | O75382 |
| TRIM31 | tripartite motif containing 31 | RING | 62865604 | Q9BZY9 |
| TRIM32 | tripartite motif containing 32 | RING | 153792582 | Q13049 |
| TRIM33 | tripartite motif containing 33 | RING | 74027251 | Q9UPN9 |
| TRIM34 | tripartite motif-containing protein 34 isoform 3 | RING | 145275183 | Q9BYJ4 |
| TRIM35 | tripartite motif-containing protein 35 isoform 2 | RING | 751368110 | Q9UPQ4 |
| TRIM36 | tripartite motif containing 36 | RING | 663429604 | Q9NQ86 |
| TRIM37 | tripartite motif containing 37 | RING | 52487176 | O94972 |
| TRIM38 | tripartite motif containing 38 | RING | 5454014 | O00635 |
| TRIM39 | tripartite motif containing 39 | RING | 25777698 | Q9HCM9 |
| TRIM4 | E3 ubiquitin-protein ligase TRIM4 isoform alpha | RING | 301601615 | Q9C037 |
| TRIM40 | tripartite motif-containing protein 40 isoform a** | RING | 557636691 | Q6P9F5 |
| TRIM41 | tripartite motif containing 41 | RING | 42516574 | Q8WV44 |
| TRIM42 | tripartite motif-containing protein 42 | RING | 301500637 | Q8IWZ5 |
| TRIM43 | tripartite motif-containing protein 43 | RING | 20270353 | Q96BQ3 |
| TRIM43B | tripartite motif-containing protein 43B | RING | 256600212 | A6NCK2 |
| TRIM45 | tripartite motif-containing protein 45 isoform 2 | RING | 224548923 | Q9H8W5 |
| TRIM46 | tripartite motif-containing protein 46 isoform 6 | RING | 538260579 | Q7Z4K8 |
| TRIM47 | tripartite motif-containing protein 47 | RING | 54792146 | Q96LD4 |
| TRIM48 | tripartite motif-containing protein 48 | RING | 201862060 | Q8IWZ4 |
| TRIM49 | tripartite motif-containing protein 49 | RING | 9966829 | P0CI25 |
| TRIM49B | tripartite motif-containing protein | RING | 331028811 | A6NDI0 |
| TRIM49C | PREDICTED: tripartite motif-containing protein 49C isoform X1 | RING | 767970497 | P0CI26 |
| TRIM49D1 | PREDICTED: tripartite motif-containing protein 49D1 isoform | RING | 767970137 | C9J1S8 |
| | X1 | | | |
| TRIM5 | tripartite motif containing 5 | RING | 283046698 | Q9C035 |
| TRIM50 | tripartite motif containing 50 | RING | 527122124 | Q86XT4 |
| TRIM51 | tripartite motif-containing protein 51 | RING | 209862805 | Q9BSJ1 |
| TRIM52 | tripartite motif-containing protein 52 | RING | 14249412 | Q96A61 |
| TRIM54 | tripartite motif-containing protein 54 isoform 1 | RING | 78482627 | Q9BYV2 |
| TRIM55 | tripartite motif-containing protein 55 isoform 4 | RING | 34878852 | Q9BYV6 |
| TRIM56 | tripartite motif containing 56 | RING | 30794216 | Q9BRZ2 |
| TRIM58 | E3 ubiquitin-protein ligase TRIM58 | RING | 112421127 | Q8NG06 |
| TRIM59 | tripartite motif containing 59 | RING | 27436877 | Q8IWR1 |
| TRIM6 | tripartite motif-containing protein 6 isoform 3 | RING | 310772223 | Q9C030 |
| TRIM60 | tripartite motif-containing protein 60 | RING | 384475510 | Q495X7 |
| TRIM61 | putative tripartite motif-containing protein 61 | RING | 60099474 | Q5EBN2 |
| TRIM62 | tripartite motif containing 62 | RING | 217035095 | Q9BVG3 |
| TRIM63 | tripartite motif containing 63, E3 ubiquitin protein ligase | RING | 19924163 | Q969Q1 |
| TRIM64 | tripartite motif-containing protein 64 | RING | 210147600 | A6NGJ6 |
| TRIM64B | putative tripartite motif-containing protein 64B | RING | 256542275 | A6NI03 |
| TRIM64C | putative tripartite motif-containing protein 64C | RING | 331028829 | A6NLI5 |
| TRIM65 | tripartite motif-containing protein 65 isoform 2 | RING | 371122551 | Q6PJ69 |
| TRIM67 | tripartite motif containing 67 | RING | 134288906 | Q6ZTA4 |
| TRIM68 | tripartite motif containing 68 | RING | 751557653 | Q6AZZ1 |
| TRIM69 | tripartite motif containing 69 | RING | 667478204 | Q86WT6 |
| TRIM7 | tripartite motif-containing protein 7 isoform 2 | RING | 44680130 | Q9C029 |
| TRIM71 | tripartite motif containing 71, E3 ubiquitin protein ligase | RING | 84993742 | Q2Q1W2 |
| TRIM72 | tripartite motif-containing protein 72 | RING | 270265876 | Q6ZMU5 |
| TRIM73 | tripartite motif-containing protein 73 | RING | 65285121 | Q86UV7 |
| TRIM74 | tripartite motif-containing protein 74 | RING | 38524612 | Q86UV6 |
| TRIM75P | Putative tripartite motif-containing protein 75 | RING | 187663991 | A6NK02 |
| TRIM77 | tripartite motif-containing protein 77 isoform 2 | RING | 429836863 | I1YAP6 |
| TRIM8 | tripartite motif containing 8 | RING | 148596968 | Q9BZR9 |
| TRIM9 | tripartite motif containing 9 | RING | 190341104 | Q9C026 |
| TRIML1 | tripartite motif family-like 1 | RING | 31542779 | Q8N9V2 |
| TRIML2 | probable E3 ubiquitin-protein ligase TRIML2 isoform 2 | SPRY | 742068547 | Q8N7C3 |
| TRIP12 | thyroid hormone receptor interactor 12 | HECTc | 545746335 | Q14669 |
| TTC3 | tetratricopeptide repeat domain 3 | RING | 49640011 | P53804 |
| UBE3A | ubiquitin protein ligase E3A | HECTc | 19718766 | Q05086 |
| UBE3B | ubiquitin protein ligase E3B | HECTc | 394025737 | Q7Z3V4 |
| UBE3C | ubiquitin protein ligase E3C | HECTc | 187960100 | Q15386 |
| UBE3D | ubiquitin protein ligase E3D | HECT_2 | 750321479 | Q7Z6J8 |
| UBE4A | ubiquitination factor E4A | RING | 323362983 | Q14139 |
| UBE4B | ubiquitination factor E4B | RING | 157739864 | O95155 |
| UBOX5 | U-box domain containing 5 | RING | 389886556 | O94941 |
| UBR1 | ubiquitin protein ligase E3 component n-recognin 1 | UBR | 28372497 | Q8IWV7 |
| UBR2 | ubiquitin protein ligase E3 component n-recognin 2 | UBR | 296317280 | Q8IWV8 |
| UBR3 | ubiquitin protein ligase E3 component n-recognin 3 (putative) | UBR | 160948610 | Q6ZT12 |
| UBR4 | ubiquitin protein ligase E3 component n-recognin 4 | UBR | 82659109 | Q5T4S7 |
| UBR5 | ubiquitin protein ligase E3 component n-recognin 5 | HECTc | 544583486 | O95071 |
| UBR7 | ubiquitin protein ligase E3 component n-recognin 7 (putative) | UBR | 154426322 | Q8N806 |
| UHRF1 | ubiquitin-like with PHD and ring finger domains 1 | RING | 586798166 | Q96T88 |
| UHRF2 | ubiquitin-like with PHD and ring finger domains 2, E3 ubiquitin | RING | 23312364 | Q96PU4 |
| | protein ligase | | | |
| UNK | RING finger protein unkempt homolog | RING | 331028525 | Q9C0B0 |
| UNKL | unkempt family zinc finger-like | RING | 448824848 | Q9H9P5 |
| VPS11 | vacuolar protein sorting-associated protein 11 homolog isoform 2 | RING | 589058161 | Q9H270 |
| VPS18 | vacuolar protein sorting-associated protein 18 homolog | RING | 17978485 | Q9P253 |
| VPS41 | vacuolar protein sorting-associated protein 41 homolog isoform 1 | RING | 114199475 | P49754 |
| VPS8 | vacuolar protein sorting-associated protein 8 homolog isoform a | RING | 57863277 | Q8N3P4 |
| WDR59 | WD repeat-containing protein 59 | RING | 58331266 | Q6PJI9 |
| WDSUB1 | WD repeat, sterile alpha motif and U-box domain containing 1 | RING | 808175923 | Q8N9V3 |
| WWP1 | WW domain containing E3 ubiquitin protein ligase 1 | HECTc | 13654239 | Q9H0M0 |
| WWP2 | WW domain containing E3 ubiquitin protein ligase 2 | HECTc | 394581985 | O00308 |
| XIAP | X-linked inhibitor of apoptosis | RING | 324711009 | P98170 |
| ZBTB12 | zinc finger and BTB domain-containing protein 12 | RING | 32469509 | Q9Y330 |
| ZFP91 | E3 ubiquitin-protein ligase ZFP91 isoform 2 | zf-C2H2 | 308081964 | Q96JP5 |
| ZFPL1 | zinc finger protein-like 1 | RING | 33300635 | O95159 |
| ZNF280A | zinc finger protein 280A | HECT | 618857456 | P59817 |
| ZNF341 | zinc finger protein 341 isoform 3 | RING | 544711270 | Q9BYN7 |
| ZNF511 | zinc finger protein 511 | RING | 28274701 | Q8NB15 |
| ZNF521 | zinc finger protein 521 isoform 2 | RING | 815891044 | Q96K83 |
| ZNF598 | zinc finger protein 598 | RING | 409264581 | Q86UK7 |
| ZNF645 | zinc finger protein 645 | RING | 22749189 | Q8N7E2 |
| ZNRF1 | zinc and ring finger 1, E3 ubiquitin protein ligase | RING | 14150005 | Q8ND25 |
| ZNRF2 | zinc and ring finger 2 | RING | 23821044 | Q8NHG8 |
| ZNRF3 | zinc and ring finger 3 | RING | 332801080 | Q9ULT6 |
| ZNRF4 | zinc and ring finger 4 | RING | 150170725 | Q8WWF5 |
| Zswim2 | zinc finger, SWIM-type containing 2 | RING | 71043932 | Q8NEG5 |
| ZXDC | zinc finger protein ZXDC isoform 2 | RING | 98961138 | Q2QGD7 |

In some embodiments, the E3 ligase binding domain is viral or of viral origin. In some embodiments, the E3 ligase binding domain is of eukaryotic origin, optionally mammalian or of mammalian origin. In some embodiments, the E3 ligase binding domain is human or is of human origin. The phrase “viral origin” refers to originating from a virus but may be found in other organisms. The phrase “human origin” refers to originating from a human but may be found in other organisms. The phrase “mammalian origin” refers to originating from a mammal but may be found in other organisms.

In some embodiments, the E3 ligase binding domain is or comprises a zinc finger protein (ZnF) or zinc finger domain or motif. In some embodiments, the E3 ligase binding domain is or comprises a polypeptide that binds E3 ligase. In some embodiments, the E3 ligase binding domain is or comprises bTrCP2 or a domain thereof, FBW7 or a domain thereof, SKP2 or a domain thereof, VHL or a domain thereof, SOCS2 or a domain thereof, SPOP or a domain thereof, HBVX or a domain thereof, Vpx or a domain thereof, Vpr or a domain thereof, WHVX or a domain thereof, CHIP or a domain thereof, ZnF913K0 or a domain thereof, or any combination thereof. In some embodiments, the E3 ligase binding domain is or comprises VHL or a domain thereof, SOCS2 or a domain thereof, Vpx or a domain thereof, Vpr or a domain thereof, WHVX or a domain thereof, or any combination thereof. In some embodiments the E3 ligase binding domain is or comprises Numb or a domain thereof, Numblike or a domain thereof (Rice et al., Molecular and cellular neurosciences. 2001; 18:525-540).

In some embodiments, the E3 ligase binding domain comprises an E3 ligase recognition motif. See e.g., Li et al., Database, Volume 2021, 2021, baab010, https://doi.org/10.1093/database/baab010, particularly at Supplementary Materials and figure S1A-SID. In some embodiments, the E3 ligase binding domain comprises a KEN motif, a PPxY, a RPVAxVxPxxR (SEQ ID NO: 164) motif, or any motif present in the UbiNet 2.0 database or described in Li et al., Database, Volume 2021, 2021, baab010, https://doi.org/10.1093/database/baab010. In some embodiments, the E3 ligase binding domain is or comprises a substrate or domain thereof of an E3 ligase. In some embodiments, the E3 ligase binding domain is or comprises a substrate or a domain thereof of an E3 ligase of Table 1. Exemplary substrates include, but are not limited to, KAI1, Cyclin B, Histone H2B, Cdc20, Cyclin A, Aurora A, Securin, Skp2, Claspin, CDH1, PLK1, iNOS, Runx1, LRRK2, p21, p53, D1g, DVL1, p73, RNF8, N-Myc, C-Myc, MKK4, RIP2, Foxp3, Numb, HLA-DRbeta, MHC-I, gp190, B7-2, MHC-II, c-Jun, Erk, Delta, Jagged, Fbxo45, TSC2, Smad2, TRIP, IRAK, TP53, ELF, MTA1, FoxO1, SGK1, JAMP, paxillin, H2A, H2AX, SOD1, Caspase-8, Caspase-10, TCF/LEF, H2A, H2A.X, IkappaBalpha, Wee1, Cdc25A, beta-Catenin, Cyclin-E, c-Myc, c-Jun, FoxO1, p27, p21, PCNA, Bim, TRB3, HIPK2, PHD1/3, Smads, Mad2, NKX3.1, NEMO, Akt1, Troponin I, MyBP-C, MyLC1/2, Histone, PCNP, HIF-1alpha, ErbB4, and Oct 4 (see e.g., https://www.cellsignal.com/learn-and-support/reference-tables/ubiquitin-ligase-table). In some embodiments, the E3 ligase binding domain is or comprises an E3 ligase substrate or a domain thereof found at UbiBrowser 2.0. available at http://ubibrowser.bio-it.cn/ubibrowser_v3/home/index. In some embodiments, the E3 ligase binding domain comprises a E3 ligase chaperone protein or a domain thereof. Exemplary chaperone proteins include, without limitation, heat shock proteins (e.g., Hsp70, Hsp90, Hsp110, Hsp40, Hsp27, CDC48, BAG6 complex, Asf1, Asf1a/Asf1b, FACT, and CDC48-p97) (see e.g., Kevei et al., 2017 FEBS Letters 591(17), particularly at Table 1).

In some embodiments, the E3 ligase binding domain is or comprises an antibody or fragment thereof capable of specifically recognizing and/or binding an E3 ligase. In some embodiments, the E3 ligase binding domain is or comprises a nanobody or fragment thereof capable of specifically recognizing and/or binding an E3 ligase. In some embodiments, the E3 ligase binding domain is or comprises a variable heavy chain (e.g., a VH or VHH) capable of specifically recognizing and/or binding an E3 ligase. See e.g., Bannas et al., 2017. Front. Immunol. https://doi.org/10.3389/fimmu.2017.01603. In some embodiments, the E3 ligase binding domain is or comprises a single variable heavy chain (e.g., a VH or VHH) capable of specifically recognizing and/or binding an E3 ligase. In some embodiments, the antibody or fragment thereof or nanobody is a recombinant antibody or fragment thereof or nanobody. In some embodiments, the E3 ligase binding domain is or comprises a camelid variable heavy chain (VHH) capable of specifically recognizing and/or binding an E3 ligase. In some embodiments, the E3 ligase binding domain is or comprises a human variable heavy chain (VH) and human variable light chain (VL) capable of specifically recognizing and/or binding an E3 ligase. In some embodiments, the E3 ligase binding domain is or comprises an engineered scaffold protein configured to specifically bind an E3 ligase. In some embodiments, the E3 ligase binding domain is or comprises an antibody or a fragment thereof, a nanobody, affibody, knottin, DARPin, adnectin, anticlin, avimer, Fynomer, beta-hairpin, alphabody, or any combination thereof.

In an example embodiment, the E3 ligase binding domain comprises or consists of a sequence according to SEQ ID NO: 78, 151, 152, 153, 154, 155, 156, 157, 158, 159, 160, or 161.

### Inducible Binding

In some embodiments, the engineered bifunctional receptor inducibly binds an E3 ligase in response to a stimulus or removal of a stimulus. In some embodiments, the engineered bifunctional receptor can have enhanced binding and/or interaction with an E3 ligase in response to a stimulus. In some embodiments, the stimulus is delivered simultaneously with the engineered bifunctional receptor. In some embodiments, the stimulus is delivered before or after delivery of the engineered bifunctional receptor. In some embodiments, the stimulus is a chemical stimulus. In some embodiments, the chemical stimulus is thalidomide or a derivative thereof. In some embodiments, the thalidomide derivative is lenalidomide, pomalidomide, or 4-hydroxythalidomide. In some embodiments, the stimulus enhances or increases interaction and/or binding of the E3 ligase binding domain and the E3 ligase. In some embodiments, the E3 ligase bound is CRBN.

In some embodiments, binding of the engineered bifunctional receptor to the E3 binding domain is increased 0.001 to 1,000 fold or more in response to the stimulus, such as 0.001, to/or 0.01, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1,000 fold or more.

Without being bound by theory, in some embodiments, increased binding of an E3 ligase to the engineered bifunctional receptor can increase degradation of a bound target protein.

### Target Binding Domain

The engineered bifunctional receptor includes a target binding domain. The target binding domain can be configured to bind any target protein of interest. In some embodiments, the target binding domain can bind two or more different target proteins. In some embodiments, this does not require having the target binding domain having separate portions or sections that bind different regions on different proteins. In such embodiments, the target binding domain is configured to bind a domain, motif, or epitope that is sufficiently similar or the same between two or more different proteins such that the target binding domain binds both proteins. Exemplary target polypeptides are discussed in greater detail elsewhere herein, but it will be appreciated that the embodiments described and demonstrated herein can be extrapolated to any desired target protein.

In some embodiments, the target binding domain is or comprises an antibody or fragment thereof capable of specifically recognizing and/or binding a target polypeptide. In some embodiments, the target binding domain is or comprises a nanobody or fragment thereof capable of specifically recognizing and/or binding a target polypeptide. In some embodiments, the target binding domain is or comprises a variable heavy chain (e.g., a VH or VHH) capable of specifically recognizing and/or binding a target polypeptide. In some embodiments, the target binding domain is or comprises a single variable heavy chain (e.g., a VH or VHH) capable of specifically recognizing and/or binding a target polypeptide. In some embodiments, the antibody or fragment thereof or nanobody is a recombinant antibody or fragment thereof or nanobody. In some embodiments, the target binding domain is or comprises a camelid variable heavy chain (VHH) capable of specifically recognizing and/or binding a target polypeptide. In some embodiments, the target binding domain is or comprises a human variable heavy chain (VH) and human variable light chain (VL) capable of specifically recognizing and/or binding a target polypeptide. In some embodiments, the target binding domain is or comprises an engineered scaffold protein configured to specifically bind a target polypeptide. In some embodiments, the target binding domain is or comprises an antibody or a fragment thereof, a nanobody, affibody, knottin, DARPin, adnectin, anticlin, avimer, Fynomer, beta-hairpin, alphabody, or any combination thereof. In some embodiments, the target binding domain is a ligand for a receptor. In some embodiments, the target binding domain is a receptor for a ligand. In some embodiments, the target binding domain can specifically bind or recognize a domain or motif that can be sufficiently similar or the same between two more different proteins such that the target binding domain can specifically bind two or more different proteins.

As used herein, the term “specific binding” refers to non-covalent physical association of a first and a second moiety wherein the association between the first and second moieties is at least 2 times as strong, at least 5 times as strong as, at least 10 times as strong as, at least 50 times as strong as, at least 100 times as strong as, or stronger than the association of either moiety with most or all other moieties present in the environment in which binding occurs. Binding of two or more entities may be considered specific if the equilibrium dissociation constant, Kd, is 10⁻³ M or less, 10⁻⁴ M or less, 10⁻⁵ M or less, 10⁻⁶ M or less, 10⁻⁷ M or less, 10⁻⁸ M or less, 10⁻⁹ M or less, 10⁻¹⁰ M or less, 10⁻¹¹ M or less, or 10⁻¹² M or less under the conditions employed, e.g., under physiological conditions such as those inside a cell or consistent with cell survival. In some embodiments, specific binding can be accomplished by a plurality of weaker interactions (e.g., a plurality of individual interactions, wherein each individual interaction is characterized by a Kd of greater than 10⁻³ M). In some embodiments, specific binding, which can be referred to as “molecular recognition,” is a saturable binding interaction between two entities that is dependent on complementary orientation of functional groups on each entity. Examples of specific binding interactions include primer-polynucleotide interaction, aptamer-aptamer target interactions, antibody-antigen interactions, avidin-biotin interactions, ligand-receptor interactions, metal-chelate interactions, hybridization between complementary nucleic acids, ligand-receptor interactions etc.

In some embodiments, the target binding domain comprises or is a polypeptide or is a molecule, or other agent (e.g., small molecule), that is capable of specifically or selectively interacting with, binding with, acting on or with, or otherwise associating or recognizing a target polypeptide that is contained in, associated with, part of, coupled to, another object, complex, surface, and the like, such as a cell or cell population, tissue, organ, subcellular locale, object surface, particle etc. In some embodiments, the target binding domain is or comprises amino acids, peptides, polypeptides, nucleic acids, polynucleotides, lipids, sugars, metals, small molecule chemicals, combinations thereof, and the like.

Exemplary target binding domains include, without limitation, VhhGFP4, DNMT3L, SARA, SHP1/2, IDAX,

### Exemplary Target Polypeptides

In some embodiments, the target polypeptide is a pathogenic polypeptide. As used in this context herein the term “pathogenic polypeptide” is a polypeptide and peptides that mediate a disease or disease state, condition, or disorder or are otherwise involved in the etiology or pathology of a disease or disease state, condition, or disorder. A pathogenic polypeptide can be endogenous (i.e., produced by the subject in which the pathogenic protein is present, such as mutated or misfolded proteins, proteins with incorrect posttranslational modifications, or proteins produced in excess of normal physiologic amounts) or exogenous (i.e., produced by another organism or microbe that is present inside a subject (such as in a microbiome or virus or virus particle (infectious proteins)) or is produced outside of the subject but that is consumed, injected, absorbed by or is otherwise delivered to a subject (such as present in food, water, the environment, infectious proteins, toxic proteins produced by plants, animals, or microorganisms, released into the environment etc.). Pathogenic proteins include allergenic proteins. Pathogenic proteins include toxic proteins and peptides.

In some embodiments, the target polypeptide is a transcription factor. In some embodiments, the target polypeptide is a growth factor. In some embodiments, the target polypeptide is a peptide hormone. In some embodiments, the target polypeptide is an immunoprotein. In some embodiments, the target polypeptide is an allergenic polypeptide. In some embodiments, target polypeptide is a neuropeptide or polypeptide. In some embodiments, the target polypeptide is a signaling polypeptide or peptide.

In some embodiments, the target polypeptide is not a pathogenic polypeptide but is a polypeptide in which a reduced amount would be desirable. For example, it may be beneficial to reduce the amount of a target protein as a prevention or to enhance the performance or other efficacy of another treatment, provide a performance enhancement to the subject, or to enhance or change a characteristic of a subject (such as for cosmetic purposes or reduce an allergenic protein in an animal).

In some embodiments, the target polypeptide is in a cell, optionally a cell for cell therapy. In some embodiments, the target polypeptide(s) mediate cellular dysfunction, exhaustion, and/or transformation. In some embodiments, the target polypeptides are transcription factors, tumor suppressors that limit CAR T cells, chromatin regulators and remodelers, T cell inhibitory receptors, NK cell inhibitory receptors, and/or the like. In some embodiments, the target polypeptide(s) is/are selected from Table 2 and any combination thereof. Table 2 provides a non-exhaustive exemplary list of target polypeptides.

TABLE 2
Exemplary Target Peptides (target binding domain)

DNMT3A (DNMT3L)
SMAD2 (SARA)
SMAD3 (SARA)
PD-1 (SHP1/2)
BTLA (SHP1/2)
2B4 (SHP1/2)
TET2 (IDAX)
Tumor suppressors that limit CAR T
TET2
DNMT3A
FASL
Chromatin Regulators
EZH2
SETDB1
SMARCA4
PBRM1
ARID1A
ARID2
EZH1
JARID2
SUZ12
EED
Transcription Factors
ATF1
ATF2
ATF3
ATF4
ATF5
ATF6
ATF7
BACH1
BACH2
BATF
BATF
BATF3
BCL6
CTCF
EOMES
EPAS1
ERG
ETS1
FLI1
FOB
FOS
FOSB
FOSL1
FOSL2
FOXP3
HIF1A
HIVEP1
HIVEP3
HNF1A
HNF1B
ID2
ID3
IKZF1
IKZF2
IRF1
IRF2
IRF3
IRF4
JDP2
JUN
JUNB
JUND
NFATC1
NFATC2
NFE2
NFE2L2
NFKB1
NFKB2
NR4A1
NR4A2
NR4A3
PRDM1
REL
RELA
RELB
RUNX1
RUNX3
SMAD2
SMAD3
SMAD4
SMARCC1
STAT1
STAT3
STAT6
TBX21
TCF7
TOX
T and NK cell inhibitory receptors
ADORA2A
BTLA
CD160
CD244
CTLA4
ENTPD1
FLT1
HAVCR2
IL10RA
IL10RB
KDR
KIR2DL1
KIR2DL2
KIR2DL3
KIR2DL4
KIR2DL5
KIR2DS1
KIR2DS2
KIR2DS3
KIR2DS4
KIR2DS5
KIR3DL1
KIR3DL2
KIR3DL3
KIR3DS1
LAG3
NT5E
PDCD1
TGFBR1
TGFBR2
TIGIT

In some embodiments, the target binding domain comprises or consists of a sequence according SEQ ID NO: 38, 46, 48, 50, 76, 108, 110, 124, 132, 136, 138, 140, 145, 146, 147, 148, 149, 150; and/or any combination thereof.

### Stimulus Responsive Domain

In some embodiments, the engineered bifunctional receptor comprises a stimulus responsive domain. In some embodiments, the stimulus responsive domain is or is part of a target binding domain and/or E3 ligase binding domain. In some embodiments, the stimulus responsive domain is a separate or different domain from the target binding domain and/or E3 ligase binding domain. In some embodiments, the stimulus responsive domain is operative linked to the target binding domain and/or E3 ligase binding domain.

The stimulus responsive domain is any domain that is altered or alters or modifies another polypeptide with which it is associated or coupled, in response to being acted on, interacting with, or being bound to a stimulus. Stimuli can include, without limitation, chemical stimuli, physical stimuli, energetic stimuli, biologic stimuli, and/or the like. Exemplary chemical stimuli include, chemical compounds, molecules, elements, pH, etc. Physical stimuli include, without limitation, temperature (heat, cold), tension, torsion, compression, vibrational energy (e.g., sonic waves), etc. Energetic stimuli include, without limitation, electromagnetic energy, radiation, magnetic field, light, and/or the like. Exemplary biologic stimuli include, without limitation, proteins, nucleic acids, fats, etc.

Exemplary stimulus responsive domains include, without limitation, Elastin-like peptides and polypeptides (e.g., (VPGXG)n (SEQ ID NO: 165), where X is a guest residue consisting of any amino acid except proline)), resilin-like polypeptides (e.g., GGRPSDSYGAPGGGN)ₙ (SEQ ID NO: 166) and (AQTPSSQYGAP)ₙ (SEQ ID NO: 167)), ADADADADARARARAR (SEQ ID NO: 168) (temperature and pH), SIRELEARIRELELRIG (SEQ ID NO: 169) (temperature, salinity); YGCVAALETKIAALETKKAALETKIAALC (SEQ ID NO: 170) (temperature), DAEFRHDSGYEVHHQK (SEQ ID NO: 171) (zinc), GGXGXDX(L/F/I)X (SEQ ID NO: 172) (calcium), IQQLKNQIKQLLKQ (SEQ ID NO: 173) (porphyrin), CCCCGGGSRGD (SEQ ID NO: 174) pH, VPGXG (SEQ ID NO: 165) (temperature, pH, salt), EAALEAALELAAELAA (SEQ ID NO: 175) (pH), KAALKAALKLAAKLAA (SEQ ID NO: 176) (pH), KAALEAALKLAAELAA (SEQ ID NO: 177) (pH), EAALKAALELAAKLAA (SEQ ID NO: 178) (pH), CGGEIRALKYEIARLKQAAQAKIRALEQKIAALEGGC (SEQ ID NO: 179) (Redox state), EACARV A AACEAAARQ (SEQ ID NO: 180) (light), RVIEKTNEKFHQIEKEFSEVEGRIQDLEKYVEDTKI (SEQ ID NO: 181) (pH), ELALKAKAELELKAG (SEQ ID NO: 182) (Solvent polarity), ELLAKKALEAEALKG (SEQ ID NO: 183) (Solvent Polarity), EWAVVLVAEAKHQ (SEQ ID NO: 184) (Solvent polarity), WGKIQKLKIAKVFK (SEQ ID NO: 185) (solvent polarity), KVIKCKAAVLWEEKK (SEQ ID NO: 186) (solvent polarity), IIPTAQETWLGVLTIMEHTV (SEQ ID NO: 187) (solvent polarity), LSGGIDVVAHELTHAVTDY (SEQ ID NO: 188) (solvent polarity), PAVHASLDKFLSSVSTVL (SEQ ID NO: 189) (solvent polarity), GYQCGTITAKNVTAN (SEQ ID NO: 190) (solvent polarity), VAEAKVAEAKVAEAK (SEQ ID NO: 191) (solvent polarity), ETATKAELLAKYEATHK (SEQ ID NO: 192) (pH), ETATKAELLAKZEATHK (SEQ ID NO: 193) (pH, salt, light), IGKLKEEIDKLNR(D/N)LDDM(E/Q)DENEQLKQENKTLLKVVGKLTR (SEQ ID NO: 194) (pH/temperature), EIAQLEYEISQLEQ (SEQ ID NO: 195) (pH), KIAQLKYKISQLKQ (SEQ ID NO: 196) (pH), EIAQLEYEISQLEQEIQALES (SEQ ID NO: 197) (pH), KIQALKQKISQLKWKIQSLKQ (SEQ ID NO: 198) (pH), QATNRNTDGSTDYGILQINSR (SEQ ID NO: 199) (pH), KLEALYVLGFFGFFTLGIMLSYIR (SEQ ID NO: 200) (shear), KLEALYVLGFFGFFTLGIMLSYIR (SEQ ID NO: 201), FKFEFKFEFKFE (SEQ ID NO: 202) (salt), FKFQFKFQFKFQ (SEQ ID NO: 203) (salt), VKVKVKTKVPPTKVKTKVKV (SEQ ID NO: 204) (temperature), FEFEFKFKFEFEFKFK (SEQ ID NO: 205) (salt), VKVKVKVKVPPTKVKVKVKV (SEQ ID NO: 206) (pH), EIAQHEKEIQAIEKKIAQHEYKIQAIEEKIAQHKEKIQAIK (SEQ ID NO: 207), QQKFQFQFEQQ (SEQ ID NO: 208) (salt), QQRFEWEFEQQ (SEQ ID NO: 209) (pH), CKQLEDKIEELLSKAACKQLEDKIEELLSK (SEQ ID NO: 210) (salt), FF (solvent polarity), GN-DYEDRYYRIN:MYRYPNQVYYPVC (SEQ ID NO: 211), (PHPGGSNWGQ)₃G (SEQ ID NO: 212) (copper), bpGELAQKLEQALQKLA (SEQ ID NO: 213) (Ni²⁺, Co²⁺, Ru(II)), RARADADARARADADA (SEQ ID NO: 214) (salt), AEAEAKAKAEAFAK AK (SEQ ID NO: 215) (salt), KLDLKLJDLKLDL (SEQ ID NO: 216) (salt, pH), and others set forth in e.g., Lin and Liu. Curr Opin Biotechnol. 2016 August; 40: 56-63; Chockalingam et al., Protein Engineering, Design and Selection, Volume 20, Issue 4, APRIL 2007, Pages 155-161; and Ye et al., ChemBioChem. 2021. https://doi.org/10.1002/cbic.202100416.

In some embodiments, control of one or more activities of the engineered bifunctional receptor includes applying a stimulus to and/or removing a stimulus from the engineered bifunctional protein so as to increase or decrease or activate or deactivate one or more activities of the engineered bifunctional linker. Thus, by controlling the timing and physical location of the stimulus, the activities of the engineered bifunctional receptor or domain thereof can be controlled and/or tuned.

### Linkers

Two or more domains of the engineered bifunctional receptor can be operatively coupled via a linker. In some embodiments, the linker is a flexible linker. In some embodiments, the linker is a rigid linker. In some embodiments, the linker is a cleavable linker. In some embodiments, the linker is a Gly-Ser (GS) linker. Exemplary Gly-Ser linkers are shown, but not limited to, those in Table 3.

TABLE 3
Exemplary Gly-Ser linkers

GSGSGSG (SEQ ID NO: 217)
SGSGSGSG (SEQ ID NO: 218)
GGGS (SEQ ID NO: 219)
GGGGS (SEQ ID NO: 220)
GGSGGSGGS (SEQ ID NO: 221)
GGGGSGGGGSGGGGS (SEQ ID NO: 222)
GGGGSGGGGS (SEQ ID NO: 223)
GGGGSGGGGSGGGGSGGGGS (SEQ ID NO: 224)
GGGGSGGGGSGGGGSGGGGSGGGGS (SEQ ID NO: 225)
GGGGSGGGGSGGGGSGGGGSGGGGSGGGGS (SEQ ID NO: 226)
GGGGSGGGGSGGGGSGGGGSGGGGSGGGGSGGGGS
(SEQ ID NO: 227)
GGGGSGGGGSGGGGSGGGGSGGGGSGGGGSGGGGSGGGGS
(SEQ ID NO: 228)
GGGGSGGGGSGGGGSGGGGSGGGGSGGGGSGGGGSGGGGSGGGGS
(SEQ ID NO: 229)
GGGGSGGGGSGGGGSGGGGSGGGGSGGGGSGGGGSGGGGSGGGGSGGGGS
(SEQ ID NO: 230)
GGGGSGGGGSGGGGSGGGGSGGGGSGGGGSGGGGSGGGGSGGGGSGGGGS
GGGGS (SEQ ID NO: 231)
GGGGSGGGGSGGGGSGGGGSGGGGSGGGGSGGGGSGGGGSGGGGSGGGGS
GGGGSGGGGS (SEQ ID NO: 232)
GGSGGSGGSGGSGGSGGSGGS (SEQ ID NO: 233)
(GS)n, n = 1-10 (SEQ ID NO: 234-242)
GGSGG (SEQ ID NO: 243)
GGSGGGGG (SEQ ID NO: 244)
GGSSG (SEQ ID NO: 245)

Other exemplary linkers include, but are not limited to, (EAAAK)ₙ (SEQ ID NO: 246-248), where n=1-3, A(EAAAK)₄ALEA(EAAAK)₄A (SEQ ID NO: 249), PAPAP (SEQ ID NO: 250), AEAAAKEAAAKA (SEQ ID NO: 251), disulfide bond, VSQTSKLTR⬇AETVFPDV (SEQ ID NO: 252), PLG⬇LWA (SEQ ID NO: 253), RVL ⬇AEA (SEQ ID NO: 254); EDVVCC⬇SMSY (SEQ ID NO: 255), GGIEGR⬇GS (SEQ ID NO: 256), TRHRQPR⬇GWE (SEQ ID NO: 257), AGNRVRR⬇SVG (SEQ ID NO: 258), RRRRRRR⬇RJR (SEQ ID NO: 259), GFLG⬇(SEQ ID NO: 260), LE, a cyclopeptide linker (e.g., CRRRRRREAEAC (SEQ ID NO: 261), CRRRRRR⬇EAEAC (SEQ ID NO: 262)), (XP)n (SEQ ID NO: 263-267), where n=1-8, GGGGSLVPRGSGGGGS (SEQ ID NO: 268), GGSGGHMGSGG (SEQ ID NO: 269), GGGSEGGGSEGGGSEGGG (SEQ ID NO: 270), AAGAATAA (SEQ ID NO: 271), GGGGG (SEQ ID NO: 272), GSGGGTGGGSG (SEQ ID NO: 274), those of Table 1 or 2 of Klein et al., Protein Engineering, Design and Selection, Volume 27, Issue 10, October 2014, Pages 325-330, those found in the Linker database available at https://www.ibi.vu.nl/programs/linkerdbwww/, and Applied Microbiology and Biotechnology volume 100, pages 215-225 (2016)/.

Additional linkers and general considerations for linker choice, configuration, and design are described in e.g., Chen et al., Adv Drug Deliv Rev. 2013 Oct. 15; 65(10): 1357-1369; van Rosmalen et al., Biochemistry 2017, 56, 50, 6565-6574; Klein et al., Protein Engineering, Design and Selection, Volume 27, Issue 10, October 2014, Pages 325-330; Arai, R. Methods in Enzymology. Volume 647, 2021, Pages 209-230; Chichili et al., Protein Sci. 2013 February; 22(2): 153-167; and Grawe and Stein, 2020. Trends Biotechnol. VOLUME 39, ISSUE 7, P731-744.

In some embodiments, the linker increases stability and/or expression of the engineered bifunctional receptor. In some embodiments, the linker increases bioactivity of the engineered bifunctional receptor. In some embodiments, the linker modifies the PK of the engineered bifunctional receptor.

### Delivery

The present disclosure also provides delivery systems for introducing the engineered bifunctional receptors or component(s) thereof of the present invention to cells, tissues, organs, or organisms. A delivery system may comprise one or more delivery vehicles and/or cargos (e.g., engineered bifunctional receptors or component(s) thereof, encoding polynucleotides, vector(s), vector systems(s), co-therapeutics, and/or the like). Exemplary delivery systems and methods include those described in paragraphs [00117] to [00278] of Feng Zhang et al., (WO2016106236A1), and pages 1241-1251 and Table 1 of Lino C A et al., Delivering CRISPR: a review of the challenges and approaches, DRUG DELIVERY, 2018, VOL. 25, NO. 1, 1234-1257, which are incorporated by reference herein in their entireties.

In some embodiments, the delivery systems may be used to introduce the components of the systems and compositions to plant cells. For example, the components may be delivered to plant cells using electroporation, microinjection, aerosol beam injection of plant cell protoplasts, biolistic methods, DNA particle bombardment, and/or Agrobacterium-mediated transformation. Examples of methods and delivery systems for plants include those described in Fu et al., Transgenic Res. 2000 Feb.; 9(1):11-9; Klein R M, et al., Biotechnology. 1992; 24:384-6; Casas A M et al., Proc Natl Acad Sci USA. 1993 Dec. 1; 90(23): 11212-11216; and U.S. Pat. No. 5,563,055, Davey M R et al., Plant Mol Biol. 1989 Sep.; 13(3):273-85, which are incorporated by reference herein in their entireties.

### Cargos

The delivery systems may comprise one or more cargos. The cargos may comprise one or more engineered bifunctional receptors or component(s) thereof, encoding polynucleotides (DNA or RNA), vector(s), vector systems(s), co-therapeutics, any combinations thereof, and/or the like described herein.

In some embodiments, a cargo may comprise one or more proteins described herein together with one or more RNAs, e.g., in the form of ribonucleoprotein complexes (RNP). The ribonucleoprotein complexes may be delivered by methods and systems herein. In some cases, the ribonucleoprotein may be delivered by way of a polypeptide-based shuttle agent. In one example, the ribonucleoprotein may be delivered using synthetic peptides comprising an endosome leakage domain (ELD) operably linked to a cell penetrating domain (CPD), to a histidine-rich domain and a CPD, e.g., as describe in WO2016161516. RNP may also be used for delivering the compositions and systems to plant cells, e.g., as described in Wu J W, et al., Nat Biotechnol. 2015 November; 33(11):1162-4.

In some embodiments, the cargo(s) can be any of the engineered bifunctional receptors or component(s) thereof, encoding polynucleotides, vectors, and/or vector systems described herein.

### Physical Delivery

In some embodiments, the cargos (and in some cases, delivery vehicles) may be introduced to cells by physical delivery methods. Examples of physical methods include microinjection, electroporation, and hydrodynamic delivery. Both nucleic acid and proteins may be delivered using such methods. For example, engineered bifunctional receptor(s) may be prepared in vitro, isolated, (refolded, purified if needed), and introduced to cells. In other embodiments, an engineered bifunctional receptor encoding polynucleotide, optionally contained in a vector, is introduced to cells where the engineered bifunctional receptor can be expressed from the encoding polynucleotide.

### Microinjection

Microinjection of the cargo and/or delivery vehicle to cells can achieve high efficiency, e.g., above 90% or about 100%. In some embodiments, microinjection may be performed using a microscope and a needle (e.g., with 0.5-5.0 m in diameter) to pierce a cell membrane and deliver the cargo directly to a target site within the cell. Microinjection may be used for in vitro and ex vivo delivery.

Polynucleotides, plasmids, or other vectors, particles (including but not limited to viral and viral like particles) comprising coding sequences for an engineered bifunctional receptor protein can be microinjected. In some cases, microinjection may be used i) to deliver DNA directly to a cell nucleus, and/or ii) to deliver mRNA (e.g., in vitro transcribed) to a cell nucleus or cytoplasm, and/or iii) proteins directly to a cell nucleus and/or cytoplasm.

Microinjection may be used to generate genetically modified animals. For example, cargos may be injected into zygotes to allow for efficient germline modification, such as insertion of an engineered bifunctional receptor gene. Such an approach can yield embryos and animals containing the exogenous cargo and/or modification caused by the same (e.g., target protein depletion). Microinjection can also be used to provide transient target protein depletion in a cell, such as by providing non-integrating vectors encoding the engineered bifunctional receptors or engineered bifunctional receptor proteins to the cell.

### Electroporation

In some embodiments, the cargos and/or delivery vehicles may be delivered by electroporation. Electroporation may use pulsed high-voltage electrical currents to transiently open nanometer-sized pores within the cellular membrane of cells suspended in buffer, allowing for components with hydrodynamic diameters of tens of nanometers to flow into the cell. In some cases, electroporation may be used on various cell types and efficiently transfer cargo into cells. Electroporation may be used for in vitro and ex vivo delivery.

Electroporation may also be used to deliver the cargo into the nuclei of mammalian cells by applying specific voltage and reagents, e.g., by nucleofection. Such approaches include those described in Wu Y, et al. (2015). Cell Res 25:67-79; Ye L, et al. (2014). Proc Natl Acad Sci USA 111:9591-6; Choi P S, Meyerson M. (2014). Nat Commun 5:3728; Wang J, Quake S R. (2014). Proc Natl Acad Sci 111:13157-62. Electroporation may also be used to deliver the cargo in vivo, e.g., with methods described in Zuckermann M, et al. (2015). Nat Commun 6:7391.

### Hydrodynamic Delivery

Hydrodynamic delivery may also be used for delivering the cargos, e.g., for in vivo delivery. In some examples, hydrodynamic delivery may be performed by rapidly pushing a large volume (8-10% body weight) solution containing the cargo into the bloodstream of a subject (e.g., an animal or human), e.g., for mice, via the tail vein. As blood is incompressible, the large bolus of liquid may result in an increase in hydrodynamic pressure that temporarily enhances permeability into endothelial and parenchymal cells, allowing for cargo not normally capable of crossing a cellular membrane to pass into cells. This approach may be used for delivering naked DNA plasmids and proteins. In some embodiments, the delivered cargos may be enriched in liver, kidney, lung, muscle, and/or heart.

### Transfection

The cargos, e.g., nucleic acids and/or polypeptides, may be introduced to cells by transfection methods for introducing nucleic acids into cells. Examples of transfection methods include calcium phosphate-mediated transfection, cationic transfection, liposome transfection, dendrimer transfection, heat shock transfection, magnetofection, lipofection, impalefection, optical transfection, proprietary agent-enhanced uptake of nucleic acid.

### Transduction

The cargos, e.g., nucleic acids and/or polypeptides, can be introduced to cells by transduction by a viral or pseudoviral particle. Methods of packaging the cargos in viral particles can be accomplished using any suitable viral vector or vector systems. Such viral vector and vector systems are described in greater detail elsewhere herein. As used in this context herein “transduction” refers to the process by which foreign nucleic acids and/or proteins are introduced to a cell (prokaryote or eukaryote) by a viral or pseudo viral particle. After packaging in a viral particle or pseudo viral particle, the viral particles can be exposed to cells (e.g., in vitro, ex vivo, or in vivo) where the viral or pseudoviral particle infects the cell and delivers the cargo to the cell via transduction. Viral and pseudoviral particles can be optionally concentrated prior to exposure to target cells. In some embodiments, the virus titer of a composition containing viral and/or pseudoviral particles can be obtained and a specific titer be used to transduce cells.

### Biolistics

The cargos, e.g., nucleic acids and/or polypeptides, and/or delivery vehicles can be introduced to cells using a biolistic method or technique. The term of art “biolistic”, as used herein refers to the delivery of nucleic acids to cells by high-speed particle bombardment. In some embodiments, the cargo(s) can be attached, associated with, or otherwise coupled to particles, which the n can be delivered to the cell via a gene-gun (see e.g., Liang et al. 2018. Nat. Protocol. 13:413-430; Svitashev et al. 2016. Nat. Comm. 7:13274; Ortega-Escalante et al., 2019. Plant. J. 97:661-672). In some embodiments, the particles can be gold, tungsten, palladium, rhodium, platinum, or iridium particles.

### Implantable Devices

In some embodiments, the delivery system can include an implantable device that incorporates or is coated with a cargo and or delivery vehicle described herein. Various implantable devices are described in the art, and include any device, graft, or other composition (e.g., polymer, ceramic, hydrogel, etc.) that can be implanted into a subject.

### Delivery Vehicles

The delivery systems may comprise one or more delivery vehicles. The delivery vehicles may deliver the cargo into cells, tissues, organs, or organisms (e.g., animals or plants). The cargos may be packaged, carried, or otherwise associated with the delivery vehicles. The delivery vehicles may be selected based on the types of cargo to be delivered, and/or the delivery is in vitro and/or in vivo. Examples of delivery vehicles include vectors, viruses (e.g., virus particles), non-viral vehicles, and other delivery reagents described herein.

In some embodiments, the delivery vehicles described herein can have a greatest dimension or greatest average dimension (e.g., diameter or greatest average diameter) of less than 100 microns (μm). In some embodiments, the delivery vehicles have a greatest dimension or greatest average dimension of less than 10 μm. In some embodiments, the delivery vehicles may have a greatest dimension or greatest average dimension of less than 2000 nanometers (nm). In some embodiments, the delivery vehicles may have a greatest dimension or greatest average dimension of less than 1000 nanometers (nm). In some embodiments, the delivery vehicles may have a greatest dimension or greatest average dimension (e.g., diameter or average diameter) of less than 900 nm, less than 800 nm, less than 700 nm, less than 600 nm, less than 500 nm, less than 400 nm, less than 300 nm, less than 200 nm, less than 150 nm, or less than 100 nm, less than 50 nm. In some embodiments, the delivery vehicles may have a greatest dimension or greatest average dimension ranging between 25 nm and 200 nm.

### Particles

In some embodiments, the delivery vehicles may be or comprise particles, including but not limited to nanoparticles, microparticles, virus particles, viral-like particles, etc.

In some embodiments, the delivery vehicle may be or comprise nanoparticles (e.g., particles with a greatest dimension or greatest average dimension (e.g., diameter or greatest average diameter) no greater than 1000 nm. In some embodiments, the delivery vehicle is or is comprised of microparticles (e.g., particles with a greatest dimension or greatest average dimension (e.g., diameter or greatest average diameter) greater than 1000 nm and not greater than 1000 μm. The particles may be provided in different forms, e.g., as solid particles (e.g., metal such as silver, gold, iron, titanium), non-metal, lipid-based solids, polymers), suspensions of particles, or combinations thereof. Metal, dielectric, and semiconductor particles may be prepared, as well as hybrid structures (e.g., core-shell particles)

Nanoparticles may also be used to deliver the compositions and systems to cells, as described in WO 2008042156, US 20130185823, and WO2015089419. In general, a “nanoparticle” refers to any particle having a diameter of less than 1000 nm. In certain embodiments, nanoparticles of the invention have a greatest dimension or greatest average dimension (e.g., diameter or average diameter) of 500 nm or less. In other embodiments, nanoparticles of the invention have a greatest dimension or greatest average dimension ranging between 25 nm and 200 nm. In other embodiments, nanoparticles of the invention have a greatest dimension or greatest average dimension of 100 nm or less. In other embodiments, nanoparticles of the invention have a greatest dimension or greatest average dimensions ranging between 35 nm and 60 nm. It will be appreciated that reference made herein to particles or nanoparticles can be interchangeable, where appropriate. Nanoparticles made of semiconducting material may also be labeled quantum dots if they are small enough (typically sub 10 nm) that quantization of electronic energy levels occurs. Such nanoscale particles are used in biomedical applications as drug carriers or imaging agents and may be adapted for similar purposes in the present invention. Semi-solid and soft nanoparticles have been manufactured and are within the scope of the present invention. Nanoparticles with one half hydrophilic and the other half hydrophobic are termed Janus particles and are particularly effective for stabilizing emulsions. They can self-assemble at water/oil interfaces and act as solid surfactants.

Particle characterization (including e.g., characterizing morphology, dimension, etc.) is done using a variety of different techniques. Common techniques are electron microscopy (TEM, SEM), atomic force microscopy (AFM), dynamic light scattering (DLS), X-ray photoelectron spectroscopy (XPS), powder X-ray diffraction (XRD), Fourier transform infrared spectroscopy (FTIR), matrix-assisted laser desorption/ionization time-of-flight mass spectrometry (MALDI-TOF), ultraviolet-visible spectroscopy, dual polarization interferometry and nuclear magnetic resonance (NMR). Characterization (dimension measurements) may be made as to native particles (i.e., preloading) or after loading of the cargo (e.g., one or engineered bifunctional receptor and/or encoding polynucleotide, etc.,) may include additional carriers and/or excipients) to provide particles of an optimal size for delivery for any in vitro, ex vivo and/or in vivo application of the present invention. In certain preferred embodiments, particle dimension (e.g., diameter) characterization is based on measurements using dynamic laser scattering (DLS). Mention is made of U.S. Pat. Nos. 8,709,843; 6,007,845; 5,855,913; 5,985,309; 5,543,158; and the publication by James E. Dahlman and Carmen Barnes et al. Nature Nanotechnology (2014) published online 11 May 2014, doi:10.1038/nnano.2014.84, describing particles, methods of making and using them and measurements thereof, which can be adapted for use with the present invention.

### Vectors and Vector Systems

Also provided herein are vectors that can contain one or more of the engineered bifunctional receptors encoding polynucleotides described herein. In certain embodiments, the vector can contain one or more polynucleotides encoding one or engineered bifunctional receptors described herein. The vectors can be useful in producing bacterial, fungal, yeast, plant cells, animal cells, and transgenic animals that can express one or more engineered bifunctional receptors described herein. Within the scope of this disclosure are vectors containing one or more of the polynucleotide sequences described herein. One or more of the polynucleotides that encode one or more engineered bifunctional receptors or component thereof described herein can be included in a vector or vector system. The vectors and/or vector systems can be used, for example, to express one or more of the polynucleotides in a cell, such as a producer cell, to produce engineered bifunctional receptor encoding polynucleotide or polypeptide containing virus or virus-like particles described elsewhere herein. The vectors and/or vector systems can be used, for example, to express one or more of the polynucleotides encoding an engineered bifunctional receptor in a cell, such as a producer cell, to produce engineered bifunctional receptor encoding polypeptides that can optionally be separated, isolated, purified, and/or otherwise harvested for subsequent delivery to a cell. Other uses for the vectors and vector systems described herein are also within the scope of this disclosure. In general, and throughout this specification, the term “vector” refers to a tool that allows or facilitates the transfer of an entity from one environment to another. In some contexts which will be appreciated by those of ordinary skill in the art, “vector” can be a term of art to refer to a nucleic acid molecule capable of transporting another nucleic acid to which it has been linked. A vector can be a replicon, such as a plasmid, phage, or cosmid, into which another DNA segment may be inserted so as to bring about the replication of the inserted segment. Generally, a vector is capable of replication when associated with the proper control elements.

Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can be composed of a nucleic acid (e.g., a polynucleotide) of the invention in a form suitable for expression of the nucleic acid in a host cell, which means that the recombinant expression vectors include one or more regulatory elements, which can be selected on the basis of the host cells to be used for expression, that is operatively-linked to the nucleic acid sequence to be expressed. Within a recombinant expression vector, “operably linked” and “operatively-linked” are used interchangeably herein and further defined elsewhere herein. In the context of a vector, the term “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). Advantageous vectors include lentiviruses and adeno-associated viruses, and types of such vectors can also be selected for targeting particular types of cells. These and other embodiments of the vectors and vector systems are described elsewhere herein.

In some embodiments, the vector can be a bicistronic vector. In some embodiments, a bicistronic vector can be used for one or more elements of the engineered bifunctional receptors described herein. In some embodiments, expression of elements of the engineered bifunctional receptors described herein can be driven by the CBh promoter or other ubiquitous promoter. Where the element of the engineered bifunctional receptor polynucleotide is an RNA, its expression can be driven by a Pol III promoter, such as a U6 promoter. In some embodiments, the two are combined. In some embodiments, the engineered bifunctional receptor encoding polynucleotides are expressed from an inducible promoter. Inducible promoters are described in greater detail elsewhere herein.

In some embodiments, a vector capable of delivering a polynucleotide encoding an engineered bifunctional receptor protein or component thereof to a cell can be composed of or contain a minimal promoter operably linked to a polynucleotide sequence encoding the engineered bifunctional receptor protein or component thereof, where the length of the vector sequence comprising the minimal promoters and polynucleotide sequences is less than about 4.4Kb. In an embodiment, the vector can be a viral vector. In certain embodiments, the viral vector is an adeno-associated virus (AAV) or an adenovirus vector. In some embodiments, a lentiviral vector for delivery of a polynucleotide encoding an engineered bifunctional receptor protein or component thereof to a cell can be composed of or contain a promoter (e.g., an inducible, constitutive, or other promoter) operatively linked to the polynucleotide encoding an engineered bifunctional receptor protein or component thereof.

These and others are further detailed and described elsewhere herein.

### Cell-based Vector Amplification and Expression

Vectors may be introduced and propagated in a eukaryotic or prokaryotic cell. In some embodiments, a prokaryote is used to amplify copies of a vector to be introduced into a eukaryotic cell or as an intermediate vector in the production of a vector to be introduced into a eukaryotic cell (e.g., amplifying a plasmid as part of a viral vector packaging system). The vectors can be viral-based or non-viral based. In some embodiments, a prokaryote is used to amplify copies of a vector and express one or more nucleic acids, such as to provide a source of one or more proteins for delivery to a host cell or host organism.

Vectors can be designed for expression of one or more elements of the engineered bifunctional receptor described herein (e.g., nucleic acid transcripts, proteins, enzymes, and combinations thereof) in a suitable host cell. In some embodiments, the suitable host cell is a prokaryotic cell. Suitable host cells include, but are not limited to, bacterial cells, yeast cells, insect cells, and mammalian cells. In some embodiments, the suitable host cell is a eukaryotic cell.

In some embodiments, the suitable host cell is a suitable bacterial cell. Suitable bacterial cells include, but are not limited to, bacterial cells from the bacteria of the species Escherichia coli. Many suitable strains of E. coli are known in the art for expression of vectors. These include, but are not limited to Pir1, Stbl2, Stbl3, Stbl4, TOP10, XL1 Blue, and XL10 Gold.

In some embodiments, the host cell is a suitable insect cell. Suitable insect cells include those from Spodoptera frugiperda. Suitable strains of S. frugiperda cells include, but are not limited to, Sf9 and Sf21. In some embodiments, the host cell is a suitable yeast cell. In some embodiments, the yeast cell can be from Saccharomyces cerevisiae. In some embodiments, the host cell is a suitable mammalian cell. Many types of mammalian cells have been developed to express vectors. Suitable mammalian cells include, but are not limited to, HEK293, Chinese Hamster Ovary Cells (CHOs), mouse myeloma cells, HeLa, U2OS, A549, HT1080, CAD, P19, NIH 3T3, L929, N2a, MCF-7, Y79, SO-Rb50, HepG G2, DIKX-X11, J558L, Baby hamster kidney cells (BHK), and chicken embryo fibroblasts (CEFs). Suitable host cells are discussed further in Goeddel, GENE EXPRESSION TECHNOLOGY: METHODS IN ENZYMOLOGY 185, Academic Press, San Diego, Calif. (1990).

In some embodiments, the vector can be a yeast expression vector. Examples of vectors for expression in yeast Saccharomyces cerevisiae include pYepSec1 (Baldari, et al., 1987. EMBO J. 6: 229-234), pMFa (Kuijan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.). As used herein, a “yeast expression vector” refers to a nucleic acid that contains one or more sequences encoding an RNA and/or polypeptide and may further contain any desired elements that control the expression of the nucleic acid(s), as well as any elements that enable the replication and maintenance of the expression vector inside the yeast cell. Many suitable yeast expression vectors and features thereof are known in the art; for example, various vectors and techniques are illustrated in in Yeast Protocols, 2nd edition, Xiao, W., ed. (Humana Press, New York, 2007) and Buckholz, R. G. and Gleeson, M. A. (1991) Biotechnology (NY) 9(11): 1067-72. Yeast vectors can contain, without limitation, a centromeric (CEN) sequence, an autonomous replication sequence (ARS), a promoter, such as an RNA Polymerase III promoter, operably linked to a sequence or gene of interest, a terminator such as an RNA polymerase III terminator, an origin of replication, and a marker gene (e.g., auxotrophic, antibiotic, or other selectable markers). Examples of expression vectors for use in yeast may include plasmids, yeast artificial chromosomes, 2 plasmids, yeast integrative plasmids, yeast replicative plasmids, shuttle vectors, and episomal plasmids.

In some embodiments, the vector is a baculovirus vector or expression vector and can be suitable for expression of polynucleotides and/or proteins in insect cells. In some embodiments, the suitable host cell is an insect cell. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39). rAAV (recombinant Adeno-associated viral) vectors are preferably produced in insect cells, e.g., Spodoptera frugiperda Sf9 insect cells, grown in serum-free suspension culture. Serum-free insect cells can be purchased from commercial vendors, e.g., Sigma Aldrich (EX-CELL 405).

In some embodiments, the vector is a mammalian expression vector. In some embodiments, the mammalian expression vector is capable of expressing one or more polynucleotides and/or polypeptides described herein in a mammalian cell. Examples of mammalian expression vectors include, but are not limited to, pCDM8 (Seed, 1987. Nature 329: 840) and pMT2PC (Kaufman, et al., 1987. EMBO J. 6: 187-195). The mammalian expression vector can include one or more suitable regulatory elements capable of controlling expression of the one or more polynucleotides and/or proteins in the mammalian cell. For example, commonly used promoters are derived from polyoma, adenovirus 2, cytomegalovirus, simian virus 40, and others disclosed herein and known in the art. More detail on suitable regulatory elements is described elsewhere herein.

For other suitable expression vectors and vector systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In some embodiments, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Baneiji, et al., 1983i. Cell33: 729-740; Queen and Baltimore, 1983i. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546). With regards to these prokaryotic and eukaryotic vectors, mention is made of U.S. Pat. No. 6,750,059, the contents of which are incorporated by reference herein in their entirety. Other embodiments can utilize viral vectors, with regards to which mention is made of U.S. patent application Ser. No. 13/092,085, the contents of which are incorporated by reference herein in their entirety. Tissue-specific regulatory elements are known in the art and in this regard, mention is made of U.S. Pat. No. 7,776,321, the contents of which are incorporated by reference herein in their entirety. In some embodiments, a regulatory element can be operably linked to one or more elements of an engineered bifunctional receptor so as to drive expression of the one or more elements of the engineered bifunctional receptor described herein.

In some embodiments, the vector can be a fusion vector or fusion expression vector. In some embodiments, fusion vectors add a number of amino acids to a protein encoded therein, such as to the amino terminus, carboxy terminus, or both of a recombinant protein. Such fusion vectors can serve one or more purposes, such as: (i) to increase expression of recombinant protein; (ii) to increase the solubility of the recombinant protein; and (iii) to aid in the purification of the recombinant protein by acting as a ligand in affinity purification. In some embodiments, expression of polynucleotides (such as non-coding polynucleotides) and proteins in prokaryotes can be carried out in Escherichia coli with vectors containing constitutive or inducible promoters directing the expression of either fusion or non-fusion polynucleotides and/or proteins. In some embodiments, the fusion expression vector can include a proteolytic cleavage site, which can be introduced at the junction of the fusion vector backbone or other fusion moiety and the recombinant polynucleotide or protein to enable separation of the recombinant polynucleotide or protein from the fusion vector backbone or other fusion moiety subsequent to purification of the fusion polynucleotide or protein. Such enzymes, and their cognate recognition sequences, include Factor Xa, thrombin and enterokinase. Example fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein. Examples of suitable inducible non-fusion E. coli expression vectors include pTrc (Amrann et al., (1988) Gene 69:301-315) and pET lid (Studier et al., GENE EXPRESSION TECHNOLOGY: METHODS IN ENZYMOLOGY 185, Academic Press, San Diego, Calif. (1990) 60-89).

In some embodiments, one or more vectors driving expression of one or more elements of an engineered bifunctional receptor described herein are introduced into a host cell such that expression of the elements of the engineered delivery system described herein direct formation a CRISPR-Cas complex at one or more target sites. For example, a CRISPR-Cas effector protein described herein and a nucleic acid component (e.g., a guide polynucleotide) can each be operably linked to separate regulatory elements on separate vectors. RNA(s) of all or of different elements of an engineered bifunctional receptor described herein can be delivered to an animal, plant, microorganism or cell thereof to produce an animal (e.g., a mammal, reptile, avian, etc.), plant, microorganism or cell thereof that constitutively, inducibly, or conditionally expresses all or different elements of the engineered bifunctional receptor described herein and/or contains one or more cells that incorporates and/or expresses one or more elements of the engineered bifunctional receptor described herein.

In some embodiments, two or more of the elements expressed from the same or different regulatory element(s), can be combined in a single vector, with one or more additional vectors providing any components of the system not included in the first vector. Engineered bifunctional receptor system encoding polynucleotides that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In some embodiments, a single promoter drives expression of a transcript encoding one or more engineered bifunctional receptor proteins or components thereof, embedded within one or more intron sequences (e.g., each in a different intron, two or more in at least one intron, or all in a single intron). In some embodiments, the engineered bifunctional receptor polynucleotides can be operably linked to and expressed from the same promoter.

### Cell-Free Vector and Polynucleotide Expression

In some embodiments, the polynucleotide encoding one or more engineered bifunctional receptors or components thereof are expressed from a vector or other suitable polynucleotide in a cell-free in vitro system. In other words, the polynucleotide, optionally contained in a vector, can be transcribed and optionally translated in vitro. In vitro transcription/translation systems and appropriate vectors are generally known in the art and commercially available. Generally, in vitro transcription and in vitro translation systems replicate the processes of RNA and protein synthesis, respectively, outside of the cellular environment. Vectors and suitable polynucleotides for in vitro transcription can include T7, SP6, T3, promoter regulatory sequences that can be recognized and acted upon by an appropriate polymerase to transcribe the polynucleotide or vector.

In vitro translation can be stand-alone (e.g., translation of a purified polyribonucleotide) or linked/coupled to transcription. In some embodiments, the cell-free (or in vitro) translation system can include extracts from rabbit reticulocytes, wheat germ, and/or E. coli. The extracts can include various macromolecular components that are needed for translation of exogenous RNA (e.g., 70S or 80S ribosomes, tRNAs, aminoacyl-tRNA, synthetases, initiation, elongation factors, termination factors, etc.). Other components can be included or added during the translation reaction, including but not limited to, amino acids, energy sources (ATP, GTP), energy regenerating systems (creatine phosphate and creatine phosphokinase (eukaryotic systems)) (phosphoenolpyruvate and pyruvate kinase for bacterial systems), and other co-factors (Mg2+, K+, etc.). As previously mentioned, in vitro translation can be based on RNA or DNA starting material. Some translation systems can utilize an RNA template as starting material (e.g., reticulocyte lysates and wheat germ extracts). Some translation systems can utilize a DNA template as a starting material (e.g., E coli-based systems). In these systems, transcription and translation are coupled and DNA is first transcribed into RNA, which is subsequently translated. Suitable standard and coupled cell-free translation systems are generally known in the art and are commercially available.

### Vector Features

The vectors can include additional features that can confer one or more functionalities to the vector, the polynucleotide to be delivered, a virus particle produced there from, or polypeptide expressed thereof. Such features include, but are not limited to, regulatory elements, selectable markers, molecular identifiers (e.g., molecular barcodes), stabilizing elements, and the like. It will be appreciated by those skilled in the art that the design of the expression vector and additional features included can depend on such factors as the choice of the host cell to be transformed, the level of expression desired, etc.

### Regulatory Elements

In certain embodiments, the polynucleotides and/or vectors thereof described herein (such as the engineered bifunctional receptor polynucleotides of the present invention) can include one or more regulatory elements that can be operatively linked to the polynucleotide. The term “regulatory element” is intended to include promoters, enhancers, internal ribosomal entry sites (IRES), other expression control elements (e.g., transcription termination signals, such as polyadenylation signals and poly-U sequences) and cellular localization signals (e.g., nuclear localization signals). Such regulatory elements are described, for example, in Goeddel, GENE EXPRESSION TECHNOLOGY: METHODS IN ENZYMOLOGY 185, Academic Press, San Diego, Calif (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter can direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) (see, e.g., Boshart et al, Cell, 41:521-530 (1985)), the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981).

In some embodiments, the regulatory sequence can be a regulatory sequence described in U.S. Pat. No. 7,776,321, U.S. Pat. Pub. No. 2011/0027239, and International Patent Publication No. WO 2011/028929, the contents of which are incorporated by reference herein in their entirety. In some embodiments, the vector can contain a minimal promoter. In some embodiments, the minimal promoter is the Mecp2 promoter, tRNA promoter, or U6. In a further embodiment, the minimal promoter is tissue specific. In some embodiments, the length of the vector polynucleotide, the minimal promoters and polynucleotide sequences is less than 4.4Kb.

To express a polynucleotide, the vector can include one or more transcriptional and/or translational initiation regulatory sequences, e.g., promoters, that direct the transcription of the gene and/or translation of the encoded protein in a cell. In some embodiments a constitutive promoter may be employed. Suitable constitutive promoters for mammalian cells are generally known in the art and include, but are not limited to SV40, CAG, CMV, EF-1α, β-actin, RSV, and PGK. Suitable constitutive promoters for bacterial cells, yeast cells, and fungal cells are generally known in the art, such as a T-7 promoter for bacterial expression and an alcohol dehydrogenase promoter for expression in yeast.

In some embodiments, the regulatory element can be a regulated promoter. “Regulated promoter” refers to promoters that direct gene expression not constitutively, but in a temporally- and/or spatially-regulated manner, and includes tissue-specific, tissue-preferred and inducible promoters. Regulated promoters include conditional promoters and inducible promoters. In some embodiments, conditional promoters can be employed to direct expression of a polynucleotide in a specific cell type, under certain environmental conditions, and/or during a specific state of development. Suitable tissue specific promoters can include, but are not limited to, liver specific promoters (e.g., APOA2, SERPIN A1 (hAAT), CYP3A4, and MIR122), pancreatic cell promoters (e.g., INS, IRS2, Pdx1, Alx3, Ppy), cardiac specific promoters (e.g., Myh6 (alpha MHC), MYL2 (MLC-2v), TNI3 (cTnl), NPPA (ANF), Slc8a1 (Ncx1)), central nervous system cell promoters (SYN1, GFAP, INA, NES, MOBP, MBP, TH, FOXA2 (HNF3 beta)), skin cell specific promoters (e.g., FLG, K14, TGM3), immune cell specific promoters, (e.g., ITGAM, CD43 promoter, CD14 promoter, CD45 promoter, CD68 promoter), urogenital cell specific promoters (e.g., Pbsn, Upk2, Sbp, Ferl14), endothelial cell specific promoters (e.g., ENG), pluripotent and embryonic germ layer cell specific promoters (e.g., Oct4, NANOG, Synthetic Oct4, T brachyury, NES, SOX17, FOXA2, MIR122), and muscle cell specific promoter (e.g., Desmin). Other tissue and/or cell specific promoters are generally known in the art and are within the scope of this disclosure.

Inducible/conditional promoters can be positively inducible/conditional promoters (e.g., a promoter that activates transcription of the polynucleotide upon appropriate interaction with an activated activator, or an inducer (compound, environmental condition, or other stimulus, such as a transcription factor released upon activation of a receptor (e.g., a membrane bound or non-membrane bound receptor) in response to a stimulus or biding of a receptor ligand) or a negative/conditional inducible promoter (e.g., a promoter that is repressed (e.g., bound by a repressor) until the repressor condition of the promoter is removed (e.g., inducer binds a repressor bound to the promoter stimulating release of the promoter by the repressor or removal of a chemical repressor from the promoter environment). The inducer can be a compound, environmental condition, or other stimulus. Thus, inducible/conditional promoters can be responsive to any suitable stimuli such as chemical, biological, or other molecular agents, temperature, light, and/or pH. Suitable inducible/conditional promoters include, but are not limited to, Tet-On, Tet-Off, Lac promoter, pBad, AlcA, LexA, Hsp70 promoter, Hsp90 promoter, pDawn, XVE/OlexA, GVG, and pOp/LhGR.

In some embodiments, expression of a polynucleotide that is optionally in a vector, is regulated by a synthetic genetic circuit of which the regulatory element(s) are a part. In some embodiments, the synthetic genetic circuit is configured as a switch (e.g., on/off), an oscillator, a cascade, or other configuration. In some embodiments, the synthetic genetic circuit comprises one or more positive and/or negative feedback loops. In some embodiments, the synthetic genetic circuit comprises one or more actuator components that facilitate positive or negative regulation of transcription of a polynucleotide, such as one encoding an engineered bifunctional receptor of the present invention. The actuator component(s) contain or are operably and/or functionally coupled to a DNA-interaction (typically binding) component that recognizes and interacts with a promoter sequence to activate, potentiate, stop, or repress transcription from that promoter. In some embodiments, the actuator and DNA-interaction components are part of the same molecule The synthetic genetic circuit also contains a sensor component that is configured to interact with (e.g., bind) and/or be responsive to a stimulus, typically an exogenous stimulus. In some embodiments, the sensor is operably and/or functionally coupled to the actuator. In some embodiments, the actuator and sensor are the same component. In other words, in some embodiments, the synthetic genetic circuit contains a bifunctional sensor/actuator component. In operation, when the sensor interacts with and/or responds to a stimulus (e.g., a ligand binding a receptor, light, heat, cold, etc.) the actuator and/or DNA-interaction components interact and/or bind with a promoter and stimulate (or repress) transcription of a polynucleotide. Exemplary synthetic genetic circuits for transcriptional control are described in e.g., Ausländer, S., and Fussenegger, M. (2013). Trends Biotechnol. 31, 155-168. doi: 10.1016/j.tibtech.2012.11.006; Khalil eta 1., (2012). Cell 150, 647-658. doi: 10.1016/j.cell.2012.05.045; Sanjana et al., (2012). Nat. Protoc. 7, 171-192. doi: 10.1038/nprot.2011.431; Li et al., (2015). Nat. Chem. Biol. 11, 207-213. doi: 10.1038/nchembio.1736; Bikard et al., Nucleic Acids Res. 41, 7429-7437. doi: 10.1093/nar/gkt520; Qi et al., (2013) Cell 152, 1173-1183. doi: 10.1016/j.cell.2013.02.022; Ferry et al., (2017) Nat. Commun. 8:14633. doi: 10.1038/ncomms14633; Jain, K. K., (2013) Med. Princ. Pract. 22, 209-219. doi: 10.1159/000341794; Lebar and Jerala. (2016) ACS Synth. Biol. 5, 1050-1058. doi: 10.1021/acssynbio.5b00259; Lucks et al., (2011) Proc. Natl. Acad. Sci. U.S.A. 108, 8617-8622. doi: 10.1073/pnas.1015741108; Wei et al., (2012). Nature 488, 384-388. doi: 10.1038/naturel 1259; Santos-Moreno and Schaerli. Biochem Soc Trans. 2020. 48(5): 1979-1993; Saltepe et al., ACS Sens. 2018, 3, 1, 13-26; Roybal et al., Cell. 167(2):419-432. (2016); Hernandez-Lopez et al., 2021. Science. 371(6534):eabc1855; Yang et al., 2021. PNAS. 118 (34) e2106612118 https://doi.org/10.1073/pnas.2106612118; Chakravarti et al., ACS Synth Biol. 2019 Aug. 16; 8(8): 1744-1754; Kitada et al., Science. 2018. Feb. 9; 359(6376): eaad1067; Huang et al., Sci. Adv. 2020. 6(8) DOI: 10.1126/sciadv.aay9209; Brandt et al., 2020. Front. Immunol., https://doi.org/10.3389/fimmu.2020.00326; Mansouri and Fussenegger. 2022. Protein & Cell. 13:476-489 and can be adapted for use to control expression of the polynucleotides of the present invention.

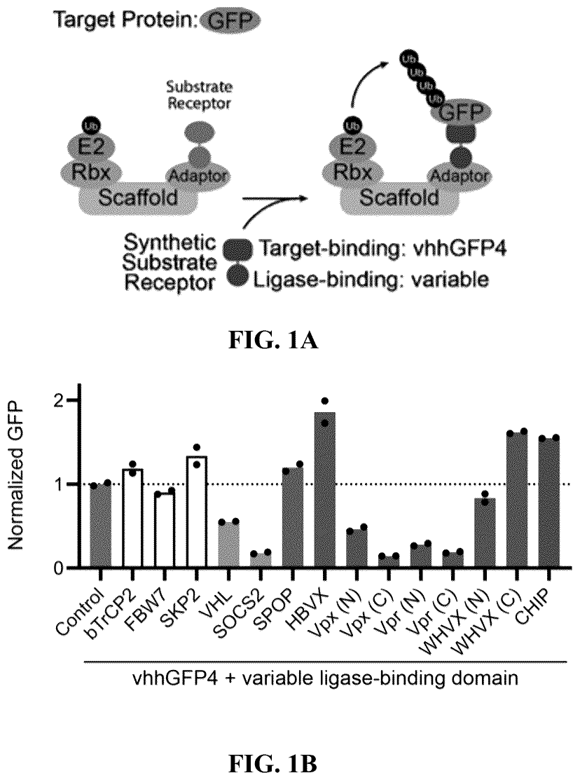

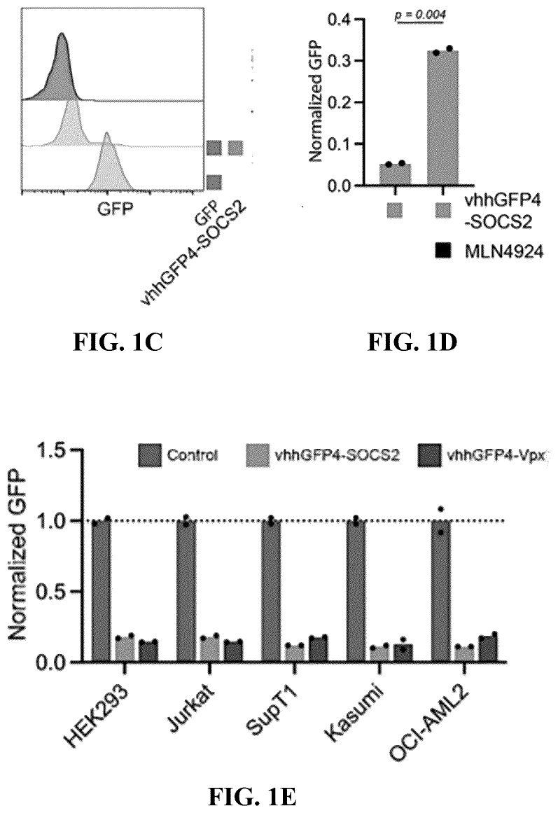

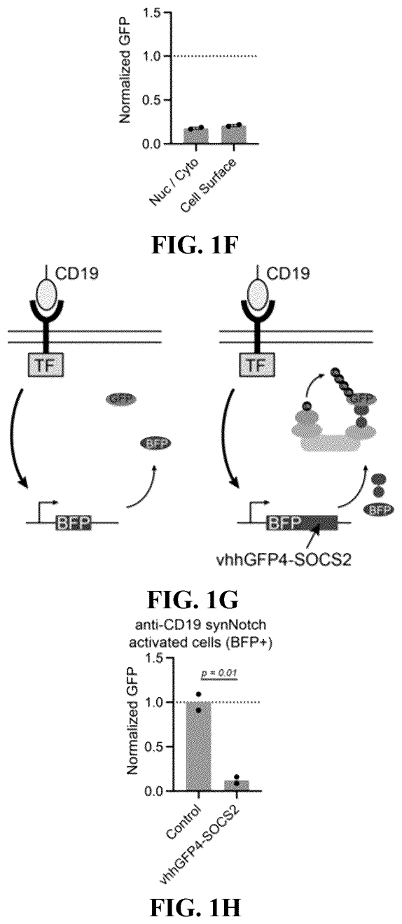

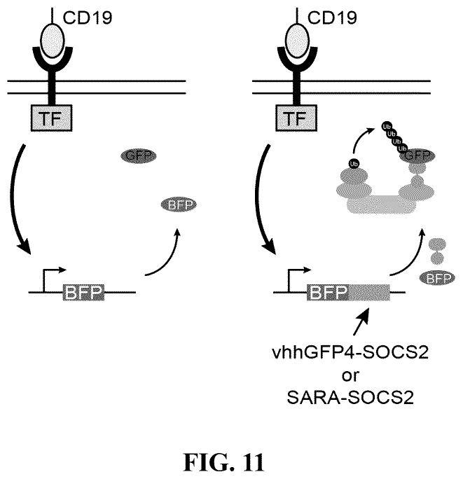

In some embodiments, the synthetic genetic circuit comprises a SynNotch Receptor. In some embodiments, the SyNotch receptor is capable of binding CD-19. In some embodiments, the SynNotch receptor is coupled to a transcription factor that binds a promoter that is operatively coupled to the engineered bifunctional receptor encoding polynucleotide. In some embodiments, the SynNotch Receptor, in response to binding CD-19, releases the transcription factor which then binds the promoter that is operatively coupled to the engineered bifunctional receptor encoding polynucleotide and activates transcription of the engineered bifunctional receptor encoding polynucleotide. See also e.g., FIGS. 1G and 11.

In some exemplary embodiment, the synthetic genetic circuit comprises a programmable RNA-guided nuclease (e.g., a TALE, Zinc Finger Nuclease, IscB, Omega, system, or a CRISPR-Cas system nuclease) that facilitates control of transcription of the engineered bifunctional receptor encoding polynucleotide.

Where expression in a plant cell is desired, the polynucleotide(s) encoding an engineered bifunctional receptor described herein are typically placed under control of a plant promoter, i.e., a promoter operable in plant cells. The use of different types of promoters is envisaged.

A constitutive plant promoter is a promoter that is able to express the open reading frame (ORF) that it controls in all or nearly all of the plant tissues during all or nearly all developmental stages of the plant (referred to as “constitutive expression”). One non-limiting example of a constitutive promoter is the cauliflower mosaic virus 35S promoter. Different promoters may direct the expression of a gene in different tissues or cell types, or at different stages of development, or in response to different environmental conditions. In particular embodiments, the polynucleotide(s) encoding an engineered bifunctional receptor are expressed under the control of a constitutive promoter, such as the cauliflower mosaic virus 35S promoter issue-preferred promoters can be utilized to target enhanced expression in certain cell types within a particular plant tissue, for instance vascular cells in leaves or roots or in specific cells of the seed. Examples of particular promoters for use in expressing the polynucleotide(s) encoding an engineered bifunctional receptor are found in Kawamata et al., (1997) Plant Cell Physiol 38:792-803; Yamamoto et al., (1997) Plant J 12:255-65; Hire et al, (1992) Plant Mol Biol 20:207-18,Kuster et al, (1995) Plant Mol Biol 29:759-72, and Capana et al., (1994) Plant Mol Biol 25:681-91.

Examples of promoters that are inducible and that can allow for spatiotemporal control of gene editing or gene expression may use a form of energy. The form of energy may include but is not limited to sound energy, electromagnetic radiation, chemical energy and/or thermal energy. Examples of inducible systems include tetracycline inducible promoters (Tet-On or Tet-Off), small molecule two-hybrid transcription activations systems (FKBP, ABA, etc.), or light inducible systems (Phytochrome, LOV domains, or cryptochrome)., such as a Light Inducible Transcriptional Effector (LITE) that direct changes in transcriptional activity in a sequence-specific manner. The components of a light inducible system may include one or more the polynucleotide(s) encoding an engineered bifunctional receptor, a light-responsive cytochrome heterodimer (e.g., from Arabidopsis thaliana), and a transcriptional activation/repression domain. In some embodiments, the vector can include one or more of the inducible DNA binding proteins provided in International Patent Publication No. WO 2014/018423 and US Patent Publication Nos., 2015/0291966, 2017/0166903, 2019/0203212, which describe e.g., embodiments of inducible DNA binding proteins and methods of use and can be adapted for use with the present invention.

In some embodiments, transient or inducible expression can be achieved by including, for example, chemical-regulated promoters, i.e., whereby the application of an exogenous chemical induces gene expression. Modulation of gene expression can also be obtained by including a chemical-repressible promoter, where application of the chemical represses gene expression. Chemical-inducible promoters include, but are not limited to, the maize ln2-2 promoter, activated by benzene sulfonamide herbicide safeners (De Veylder et al., (1997) Plant Cell Physiol 38:568-77), the maize GST promoter (GST-ll-27, WO93/01294), activated by hydrophobic electrophilic compounds used as pre-emergent herbicides, and the tobacco PR-1 a promoter (Ono et al., (2004) Biosci Biotechnol Biochem 68:803-7) activated by salicylic acid. Promoters which are regulated by antibiotics, such as tetracycline-inducible and tetracycline-repressible promoters (Gatz et al., (1991) Mol Gen Genet 227:229-37; U.S. Pat. Nos. 5,814,618 and 5,789,156) can also be used herein.

In some embodiments, the polynucleotide, vector or system thereof can include one or more elements capable of translocating and/or expressing the polynucleotide(s) encoding an engineered bifunctional receptor to/in a specific cell component or organelle. Such organelles can include, but are not limited to, nucleus, ribosome, endoplasmic reticulum, Golgi apparatus, chloroplast, mitochondria, vacuole, lysosome, cytoskeleton, plasma membrane, cell wall, peroxisome, centrioles, etc. Such regulatory elements can include, but are not limited to, nuclear localization signals (examples of which are described in greater detail elsewhere herein), any such as those that are annotated in the LocSigDB database (see e.g., http://genome.unmc.edu/LocSigDB/and Negi et al., 2015. Database. 2015: bav003; doi: 10.1093/database/bav003), nuclear export signals (e.g., LXXXLXXLXL (SEQ ID NO: 274) and others described elsewhere herein), endoplasmic reticulum localization/retention signals (e.g., KDEL (SEQ ID NO: 275), KDXX, KKXX, KXX, and others described elsewhere herein; and see e.g., Liu et al. 2007 Mol. Biol. Cell. 18(3):1073-1082 and Gorleku et al., 2011. J. Biol. Chem. 286:39573-39584), mitochondria (see e.g., Cell Reports. 22:2818-2826, particularly at FIG. 2; Doyle et al. 2013. PLoS ONE 8, e67938; Funes et al. 2002. J. Biol. Chem. 277:6051-6058; Matouschek et al. 1997. PNAS USA 85:2091-2095; Oca-Cossio et al., 2003. 165:707-720; Waltner et al., 1996. J. Biol. Chem. 271:21226-21230; Wilcox et al., 2005. PNAS USA 102:15435-15440; Galanis et al., 1991. FEBS Lett 282:425-430, peroxisome (e.g. (S/A/C)-(K/R/H)-(L/A), SLK, (R/K)-(L/V/I)-XXXXX-(H/Q)-(L/A/F). Suitable protein targeting motifs can also be designed or identified using any suitable database or prediction tool, including but not limited to Minimotif Miner (http:minimotifminer.org, http://mitominer.mrc-mbu.cam.ac.uk/release-4.0/embodiment.do?name=Protein %20MTS), LocDB (see above), PTSs predictor ( ), TargetP-2.0 (http://www.cbs.dtu.dk/services/TargetP/), ChloroP (http://www.cbs.dtu.dk/services/ChloroP/); NetNES (http://www.cbs.dtu.dk/services/NetNES/), Predotar (https://urgi.versailles.inra.fr/predotar/), and SignalP (http://www.cbs.dtu.dk/services/SignalP/).

### Selectable Markers and Tags

One or more of the polynucleotide(s) encoding an engineered bifunctional receptor can be operably linked, fused to, or otherwise modified to include a polynucleotide that encodes or is a selectable marker or tag, which can be a polynucleotide or polypeptide. In some embodiments, the polypeptide encoding a polypeptide selectable marker can be incorporated in the polynucleotide(s) encoding an engineered bifunctional receptor such that the selectable marker polypeptide, when translated, is inserted between two amino acids between the N- and C-terminus of the engineered bifunctional receptor and/or at the N- and/or C-terminus of the engineered bifunctional receptor polypeptide. In some embodiments, the selectable marker or tag is a polynucleotide barcode or unique molecular identifier (UMI).

It will be appreciated that the polynucleotide encoding such selectable markers or tags can be incorporated into the polynucleotide(s) encoding an engineered bifunctional receptor described herein in an appropriate manner to allow expression of the selectable marker or tag. Such techniques and methods are described elsewhere herein and will be instantly appreciated by one of ordinary skill in the art in view of this disclosure. Many such selectable markers and tags are generally known in the art and are intended to be within the scope of this disclosure.

Suitable selectable markers and tags include, but are not limited to, affinity tags, such as chitin binding protein (CBP), maltose binding protein (MBP), glutathione-S-transferase (GST), poly(His) tag; solubilization tags such as thioredoxin (TRX) and poly(NANP), MBP, and GST; chromatography tags such as those consisting of polyanionic amino acids, such as FLAG-tag; epitope tags such as V5-tag, Myc-tag, HA-tag and NE-tag; protein tags that can allow specific enzymatic modification (such as biotinylation by biotin ligase) or chemical modification (such as reaction with FlAsH-EDT2 for fluorescence imaging), DNA and/or RNA segments that contain restriction enzyme or other enzyme cleavage sites; DNA segments that encode products that provide resistance against otherwise toxic compounds including antibiotics, such as, spectinomycin, ampicillin, kanamycin, tetracycline, Basta, neomycin phosphotransferase II (NEO), hygromycin phosphotransferase (HPT)) and the like; DNA and/or RNA segments that encode products that are otherwise lacking in the recipient cell (e.g., tRNA genes, auxotrophic markers); DNA and/or RNA segments that encode products which can be readily identified (e.g., phenotypic markers such as β-galactosidase, GUS; fluorescent proteins such as green fluorescent protein (GFP), cyan (CFP), yellow (YFP), red (RFP), luciferase, and cell surface proteins); polynucleotides that can generate one or more new primer sites for PCR (e.g., the juxtaposition of two DNA sequences not previously juxtaposed), DNA sequences not acted upon or acted upon by a restriction endonuclease or other DNA modifying enzyme, chemical, etc.; epitope tags (e.g., GFP, FLAG- and His-tags), and, DNA sequences that make a molecular barcode or unique molecular identifier (UMI), DNA sequences required for a specific modification (e.g., methylation) that allows its identification. Other suitable markers will be appreciated by those of skill in the art.

Selectable markers and tags can be operably linked to an engineered bifunctional receptor described herein via suitable linker, such as a glycine or glycine serine linkers as short as GS or GG up to (GGGGG)3 (SEQ ID NO: 276) or (GGGGS)3 (SEQ ID NO: 277). Other suitable linkers are described elsewhere herein.

The vector or vector system can include one or more polynucleotides encoding one or more targeting moieties. In some embodiments, the targeting moiety encoding polynucleotides can be included in the vector or vector system, such as a viral vector system, such that they are expressed within and/or on the virus particle(s) produced such that the virus particles can be targeted to specific cells, tissues, organs, etc. In some embodiments, the targeting moiety encoding polynucleotides can be included in the vector or vector system such that the polynucleotide(s) encoding an engineered bifunctional receptor and/or products expressed therefrom (e.g., an engineered bifunctional receptor) include the targeting moiety and can be targeted to specific cells, tissues, organs, etc. In some embodiments, such as non-viral carriers, the targeting moiety can be attached to the carrier (e.g., polymer, lipid, inorganic molecule etc.) and can be capable of targeting the carrier and any attached or associated polynucleotide(s) encoding an engineered bifunctional receptor to specific cells, tissues, organs, etc.

### Codon Optimization of Vector Polynucleotides

As described elsewhere herein, the polynucleotide(s) encoding an engineered bifunctional receptor described herein can be codon optimized. In some embodiments, one or more polynucleotides contained in a vector (“vector polynucleotides”) described herein that are in addition to an optionally codon optimized polynucleotide encoding embodiments of the engineered bifunctional receptors described herein can be codon optimized. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, PA), are also available. In some embodiments, one or more codons (e.g., 1, 2, 3, 4, 5, 10, 15, 20, 25, 50, or more, or all codons) in a sequence encoding a DNA/RNA-targeting Cas protein corresponds to the most frequently used codon for a particular amino acid. As to codon usage in yeast, reference is made to the online Yeast Genome database available at http://www.yeastgenome.org/community/codon_usage.shtml, or Codon selection in yeast, Bennetzen and Hall, J Biol Chem. 1982 Mar. 25; 257(6):3026-31. As to codon usage in plants including algae, reference is made to Codon usage in higher plants, green algae, and cyanobacteria, Campbell and Gowri, Plant Physiol. 1990 January; 92(1): 1-11.; as well as Codon usage in plant genes, Murray et al, Nucleic Acids Res. 1989 Jan. 25; 17(2):477-98; or Selection on the codon bias of chloroplast and cyanelle genes in different plant and algal lineages, Morton B R, J Mol Evol. 1998 April; 46(4):449-59.

The vector polynucleotide can be codon optimized for expression in a specific cell-type, tissue type, organ type, and/or subject type. In some embodiments, a codon optimized sequence is a sequence optimized for expression in a eukaryote, e.g., humans (i.e., being optimized for expression in a human or human cell), or for another eukaryote, such as another animal (e.g., a mammal or avian) as is described elsewhere herein. Such codon optimized sequences are within the ambit of the ordinary skilled artisan in view of the description herein. In some embodiments, the polynucleotide is codon optimized for a specific cell type. Such cell types can include, but are not limited to, epithelial cells (including skin cells, cells lining the gastrointestinal tract, cells lining other hollow organs), nerve cells (nerves, brain cells, spinal column cells, nerve support cells (e.g., astrocytes, glial cells, Schwann cells etc.), muscle cells (e.g., cardiac muscle, smooth muscle cells, and skeletal muscle cells), connective tissue cells (fat and other soft tissue padding cells, bone cells, tendon cells, cartilage cells), blood cells, stem cells and other progenitor cells, immune system cells, germ cells, and combinations thereof. Such codon optimized sequences are within the ambit of the ordinary skilled artisan in view of the description herein. In some embodiments, the polynucleotide is codon optimized for a specific tissue type. Such tissue types can include, but are not limited to, muscle tissue, connective tissue, connective tissue, nervous tissue, and epithelial tissue. Such codon optimized sequences are within the ambit of the ordinary skilled artisan in view of the description herein. In some embodiments, the polynucleotide is codon optimized for a specific organ. Such organs include, but are not limited to, muscles, skin, intestines, liver, spleen, brain, lungs, stomach, heart, kidneys, gallbladder, pancreas, bladder, thyroid, bone, blood vessels, blood, and combinations thereof. Such codon optimized sequences are within the ambit of the ordinary skilled artisan in view of the description herein.

In some embodiments, a vector polynucleotide is codon optimized for expression in particular cells, such as prokaryotic or eukaryotic cells. The eukaryotic cells may be those of or derived from a particular organism, such as a plant or a mammal, including but not limited to human, or non-human eukaryote or animal or mammal as discussed herein, e.g., mouse, rat, rabbit, dog, livestock, or non-human mammal or primate.

### Vector Construction

The vectors described herein can be constructed using any suitable process or technique. In some embodiments, one or more suitable recombination and/or cloning methods or techniques can be used to the vector(s) described herein. Suitable recombination and/or cloning techniques and/or methods can include, but not limited to, those described in U.S. Patent Publication No. US 2004/0171156 A1. Other suitable methods and techniques are described elsewhere herein.

Construction of recombinant AAV vectors is described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989). Any of the techniques and/or methods can be used and/or adapted for constructing an AAV or other vector described herein. nAAV vectors are discussed elsewhere herein.

In some embodiments, a vector comprises one or more insertion sites, such as a restriction endonuclease recognition sequence (also referred to as a “cloning site”). In some embodiments, one or more insertion sites (e.g., about or more than about 1, 2, 3, 4, 5, 6, 7, 8, 9, 10, or more insertion sites) are located upstream and/or downstream of one or more sequence elements of one or more vectors. When multiple different guide polynucleotides are used, a single expression construct may be used to target nucleic acid-targeting activity to multiple different, corresponding target sequences within a cell. For example, a single vector may comprise about or more than about 1, 2, 3, 4, 5, 6, 7, 8, 9, 10, 15, 20, or more guide polynucleotides. In some embodiments, about or more than about 1, 2, 3, 4, 5, 6, 7, 8, 9, 10, or more such guide-polynucleotide-containing vectors may be provided, and optionally delivered to a cell.

Delivery vehicles, vectors, particles, nanoparticles, formulations and components thereof for expression of the polynucleotide(s) encoding an engineered bifunctional receptor described herein are as used in the foregoing documents, such as International Patent Publication No. WO 2014/093622 (PCT/US2013/074667) and are discussed in greater detail herein.

### Viral Vectors

In some embodiments, the vector is a viral vector. The term of art “viral vector” and as used herein in this context refers to polynucleotide based vectors that contain one or more elements from or based upon one or more elements of a virus that can be capable of expressing and packaging a polynucleotide, such as the polynucleotide(s) encoding an engineered bifunctional receptor of the present invention, into a virus particle and producing said virus particle when used alone or with one or more other viral vectors (such as in a viral vector system). Viral vectors and systems thereof can be used for producing viral particles for delivery of and/or expression of the polynucleotide(s) encoding an engineered bifunctional receptor described herein. The viral vector can be part of a viral vector system involving multiple vectors. In some embodiments, systems incorporating multiple viral vectors can increase the safety of these systems. Suitable viral vectors can include retroviral-based vectors, lentiviral-based vectors, adenoviral-based vectors, adeno associated vectors, helper-dependent adenoviral (HdAd) vectors, hybrid adenoviral vectors, herpes simplex virus-based vectors, poxvirus-based vectors, and Epstein-Barr virus-based vectors. Other embodiments of viral vectors and viral particles produced therefrom are described elsewhere herein. In some embodiments, the viral vectors are configured to produce replication incompetent viral particles for improved safety of these systems.

In certain embodiments, the virus structural component, which can be encoded by one or more polynucleotides in a viral vector or vector system, comprises one or more capsid proteins including an entire capsid. In certain embodiments, such as wherein a viral capsid comprises multiple copies of different proteins, the delivery system can provide one or more of the same protein or a mixture of such proteins. For example, AAV comprises 3 capsid proteins, VP1, VP2, and VP3, thus delivery systems of the invention can comprise one or more of VP1, and/or one or more of VP2, and/or one or more of VP3. Accordingly, the present invention is applicable to a virus within the family Adenoviridae, such as Atadenovirus, e.g., Ovine atadenovirus D, Aviadenovirus, e.g., Fowl aviadenovirus A, Ichtadenovirus, e.g., Sturgeon ichtadenovirus A, Mastadenovirus (which includes adenoviruses such as all human adenoviruses), e.g., Human mastadenovirus C, and Siadenovirus, e.g., Frog siadenovirus A. Thus, a virus of within the family Adenoviridae is contemplated as within the invention with discussion herein as to adenovirus applicable to other family members. Target-specific AAV capsid variants can be used or selected. Non-limiting examples include capsid variants selected to bind to chronic myelogenous leukemia cells, human CD34 PBPC cells, breast cancer cells, cells of lung, heart, dermal fibroblasts, melanoma cells, stem cell, glioblastoma cells, coronary artery endothelial cells and keratinocytes. See, e.g., Buning et al, 2015, Current Opinion in Pharmacology 24, 94-104. From teachings herein and knowledge in the art as to modifications of adenovirus (see, e.g., U.S. Pat. Nos. 9,410,129, 7,344,872, 7,256,036, 6,911,199, 6,740,525; Matthews, “Capsid-Incorporation of Antigens into Adenovirus Capsid Proteins for a Vaccine Approach,” Mol Pharm, 8(1): 3-11 (2011)), as well as regarding modifications of AAV, the skilled person can readily obtain a modified adenovirus that has a large payload protein or a engineered bifunctional receptor, despite that heretofore it was not expected that such a large protein could be provided on an adenovirus. And as to the viruses related to adenovirus mentioned herein, as well as to the viruses related to AAV mentioned elsewhere herein, the teachings herein as to modifying adenovirus and AAV, respectively, can be applied to those viruses without undue experimentation from this disclosure and the knowledge in the art.

In some embodiments, the viral vector is configured such that when the cargo is packaged the cargo(s) (e.g., one or more polynucleotide(s) encoding an engineered bifunctional receptor and/or engineered bifunctional receptor), is external to the capsid or virus particle. In the sense that it is not inside the capsid (enveloped or encompassed with the capsid), but is externally exposed so that it can contact a target polypeptide. In some embodiments, the viral vector is configured such that all the cargo(s) are contained within the capsid after packaging.

### Split Viral Vector Systems

When the engineered bifunctional receptor viral vector or vector system (be it a retroviral (e.g., AAV) or lentiviral vector) is designed so as to position the cargo(s) (e.g., one or more engineered bifunctional receptor encoding polynucleotides or proteins) at the internal surface of the capsid once formed, the cargo(s) will fill most or all of internal volume of the capsid. In other embodiments, the engineered bifunctional receptor protein and/or polynucleotide may be modified or divided so as to occupy less of the capsid internal volume. Accordingly, in certain embodiments, the engineered bifunctional receptor protein and/or polynucleotide can be divided in two portions, where one portion is comprised in one viral particle or capsid and the second portion is comprised in a second viral particle or capsid. In certain embodiments, by splitting the engineered bifunctional receptor protein and/or polynucleotide in two portions, space is made available to link one or more heterologous domains to one or both engineered bifunctional receptor protein and/or polynucleotide portions. Such systems can be referred to as “split vector systems” or in the context of the present disclosure a “split engineered bifunctional receptor system” a “split engineered bifunctional receptor protein”, and the like. This split protein approach is also described elsewhere herein. When the concept is applied to a vector system, it describes putting pieces of the split proteins on different vectors thus reducing the payload of any one vector. This approach can facilitate delivery of systems where the total system size is close to or exceeds the packaging capacity of the vector. This is independent of any regulation of the engineered bifunctional receptor that can be achieved with a split system or split protein design.

Split engineered bifunctional receptor proteins that can be incorporated into the AAV or other vectors described herein are set forth elsewhere herein and in documents incorporated herein by reference in further detail herein. In certain embodiments, each part of a split engineered bifunctional receptor protein is attached to a member of a specific binding pair, and when bound with each other, the members of the specific binding pair maintain the parts of the engineered bifunctional receptor protein in proximity. In certain embodiments, each part of a split engineered bifunctional receptor protein is associated with an inducible binding pair. An inducible binding pair is one which is capable of being switched “on” or “off” by a protein or small molecule that binds to both members of the inducible binding pair. In general, according to the invention, engineered bifunctional receptor proteins may preferably split between domains, leaving domains intact.

### Retroviral and Lentiviral Vectors

Retroviral vectors can be composed of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Suitable retroviral vectors for the engineered bifunctional receptors can include those based upon murine leukemia virus (MuLV), gibbon ape leukemia virus (GaLV), Simian immunodeficiency virus (SIV), human immunodeficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700). Selection of a retroviral gene transfer system may therefore depend on the target tissue.

The tropism of a retrovirus can be altered by incorporating foreign envelope proteins, expanding the potential target population of target cells. Lentiviral vectors are retroviral vectors that are able to transduce or infect non-dividing cells and are described in greater detail elsewhere herein. A retrovirus can also be engineered to allow for conditional expression of the inserted transgene, such that only certain cell types are infected by the lentivirus.

Lentiviruses are complex retroviruses that have the ability to infect and express their genes in both mitotic and post-mitotic cells. Advantages of using a lentiviral approach can include the ability to transduce or infect non-dividing cells and their ability to typically produce high viral titers, which can increase efficiency or efficacy of production and delivery. Suitable lentiviral vectors include, but are not limited to, human immunodeficiency virus (HIV)-based lentiviral vectors, feline immunodeficiency virus (FIV)-based lentiviral vectors, simian immunodeficiency virus (SIV)-based lentiviral vectors, Moloney Murine Leukaemia Virus (Mo-MLV), Visna.maedi virus (VMV)-based lentiviral vector, carpine arthritis-encephalitis virus (CAEV)-based lentiviral vector, bovine immune deficiency virus (BIV)-based lentiviral vector, and Equine infectious anemia (EIAV)-based lentiviral vector. In some embodiments, an HIV-based lentiviral vector system can be used. In some embodiments, a FIV-based lentiviral vector system can be used.

In some embodiments, the lentiviral vector is an EIAV-based lentiviral vector or vector system. EIAV vectors have been used to mediate expression, packaging, and/or delivery in other contexts, such as for ocular gene therapy (see, e.g., Balagaan, J Gene Med 2006; 8: 275-285). In another embodiment, RetinoStat®, (see, e.g., Binley et al., HUMAN GENE THERAPY 23:980-991 (September 2012)), which describes RetinoStat®, an equine infectious anemia virus-based lentiviral gene therapy vector that expresses angiostatic proteins endostatin and angiostatin that is delivered via a subretinal injection for the treatment of the wet form of age-related macular degeneration. Any of these vectors described in these publications can be modified for expression and delivery of the engineered bifunctional receptor polypeptide and encoding polynucleotides described herein.

In some embodiments, the lentiviral vector or vector system thereof can be a first-generation lentiviral vector or vector system thereof. First-generation lentiviral vectors can contain a large portion of the lentivirus genome, including the gag and pol genes, other additional viral proteins (e.g., VSV-G) and other accessory genes (e.g., vif, vprm vpu, nef, and combinations thereof), regulatory genes (e.g., tat and/or rev) as well as the gene of interest between the LTRs. First generation lentiviral vectors can result in the production of virus particles that can be capable of replication in vivo, which may not be appropriate for some instances or applications.

In some embodiments, the lentiviral vector or vector system thereof can be a second-generation lentiviral vector or vector system thereof. Second-generation lentiviral vectors do not contain one or more accessory virulence factors and do not contain all components necessary for virus particle production on the same lentiviral vector. This can result in the production of a replication-incompetent virus particle and thus increase the safety of these systems over first-generation lentiviral vectors. In some embodiments, the second-generation vector lacks one or more accessory virulence factors (e.g., vif, vprm, vpu, nef, and combinations thereof). Unlike the first-generation lentiviral vectors, no single second generation lentiviral vector includes all features necessary to express and package a polynucleotide into a virus particle. In some embodiments, the envelope and packaging components are split between two different vectors with the gag, pol, rev, and tat genes being contained on one vector and the envelope protein (e.g., VSV-G) are contained on a second vector. The gene of interest, its promoter, and LTRs can be included on a third vector that can be used in conjunction with the other two vectors (packaging and envelope vectors) to generate a replication-incompetent virus particle.

In some embodiments, the lentiviral vector or vector system thereof can be a third-generation lentiviral vector or vector system thereof. Third-generation lentiviral vectors and vector systems thereof have increased safety over first- and second-generation lentiviral vectors and systems thereof because, for example, the various components of the viral genome are split between two or more different vectors but used together in vitro to make virus particles, they can lack the tat gene (when a constitutively active promoter is included up-stream of the LTRs), and they can include one or more deletions in the 3′LTR to create self-inactivating (SIN) vectors having disrupted promoter/enhancer activity of the LTR. In some embodiments, a third-generation lentiviral vector system can include (i) a vector plasmid that contains the polynucleotide of interest and upstream promoter that are flanked by the 5′ and 3′ LTRs, which can optionally include one or more deletions present in one or both of the LTRs to render the vector self-inactivating; (ii) a “packaging vector(s)” that can contain one or more genes involved in packaging a polynucleotide into a virus particle that is produced by the system (e.g., gag, pol, and rev) and upstream regulatory sequences (e.g., promoter(s)) to drive expression of the features present on the packaging vector, and (iii) an “envelope vector” that contains one or more envelope protein genes and upstream promoters. In certain embodiments, the third-generation lentiviral vector system can include at least two packaging vectors, with the gag-pol being present on a different vector than the rev gene.

In some embodiments, self-inactivating lentiviral vectors with an siRNA targeting a common exon shared by HIV tat/rev, a nucleolar-localizing TAR decoy, and an anti-CCR5-specific hammerhead ribozyme (see, e.g., DiGiusto et al. (2010) Sci Transl Med 2:36ra43) can be used with/and or adapted for the engineered bifunctional receptor polypeptides and/or encoding polynucleotides of the present invention.

In some embodiments, the pseudotype and infectivity or tropism of a lentivirus particle can be tuned by altering the type of envelope protein(s) included in the lentiviral vector or system thereof. As used herein, an “envelope protein” or “outer protein” means a protein exposed at the surface of a viral particle that is not a capsid protein. For example, envelope or outer proteins typically comprise proteins embedded in the envelope of the virus. In some embodiments, a lentiviral vector or vector system thereof can include a VSV-G envelope protein. VSV-G mediates viral attachment to an LDL receptor (LDLR) or an LDLR family member present on a host cell, which triggers endocytosis of the viral particle by the host cell. Because LDLR is expressed by a wide variety of cells, viral particles expressing the VSV-G envelope protein can infect or transduce a wide variety of cell types. Other suitable envelope proteins can be incorporated based on the host cell that a user desires to be infected by a virus particle produced from a lentiviral vector or system thereof described herein and can include, but are not limited to, feline endogenous virus envelope protein (RD 114) (see e.g., Hanawa et al. Molec. Ther. 2002 5(3) 242-251), modified Sindbis virus envelope proteins (see e.g., Morizono et al. 2010. J. Virol. 84(14) 6923-6934; Morizono et al. 2001. J. Virol. 75:8016-8020; Morizono et al. 2009. J. Gene Med. 11:549-558; Morizono et al. 2006 Virology 355:71-81; Morizono et al J. Gene Med. 11:655-663, Morizono et al. 2005 Nat. Med. 11:346-352), baboon retroviral envelope protein (see e.g., Girard-Gagnepain et al. 2014. Blood. 124: 1221-1231); Tupaia paramyxovirus glycoproteins (see e.g., Enkirch T. et al., 2013. Gene Ther. 20:16-23); measles virus glycoproteins (see e.g., Funke et al. 2008. Molec. Ther. 16(8): 1427-1436), rabies virus envelope proteins, MLV envelope proteins, Ebola envelope proteins, baculovirus envelope proteins, filovirus envelope proteins, hepatitis E1 and E2 envelope proteins, gp41 and gp120 of HIV, hemagglutinin, neuraminidase, M2 proteins of influenza virus, and combinations thereof.

In some embodiments, the tropism of the resulting lentiviral particle can be tuned by incorporating cell targeting peptides into a lentiviral vector such that the cell targeting peptides are expressed on the surface of the resulting lentiviral particle. In some embodiments, a lentiviral vector can contain an envelope protein that is fused to a cell targeting protein (see e.g., Buchholz et al. 2015. Trends Biotechnol. 33:777-790; Bender et al. 2016. PLoS Pathog. 12(e1005461); and Friedrich et al. 2013. Mol. Ther. 2013. 21: 849-859.

In some embodiments, a split-intein-mediated approach to target lentiviral particles to a specific cell type can be used (see e.g., Chamoun-Emaneulli et al. 2015. Biotechnol. Bioeng. 112:2611-2617, Ramirez et al. 2013. Protein. Eng. Des. Sel. 26:215-233. In these embodiments, a lentiviral vector can contain one half of a splicing-deficient variant of the naturally split intein from Nostoc punctiforme fused to a cell targeting peptide and the same or different lentiviral vector can contain the other half of the split intein fused to an envelope protein, such as a binding-deficient, fusion-competent virus envelope protein. This can result in production of a virus particle from the lentiviral vector or vector system that includes a split intein that can function as a molecular Velcro linker to link the cell-binding protein to the pseudotyped lentivirus particle. This approach can be advantageous for use where surface-incompatibilities can restrict the use of, e.g., cell targeting peptides.

In some embodiments, a covalent-bond-forming protein-peptide pair can be incorporated into one or more of the lentiviral vectors described herein to conjugate a cell targeting peptide to the virus particle (see e.g., Kasaraneni et al. 2018. Sci. Reports (8) No. 10990). In some embodiments, a lentiviral vector can include an N-terminal PDZ domain of InaD protein (PDZ1) and its pentapeptide ligand (TEFCA (SEQ ID NO: 278)) from NorpA, which can conjugate the cell targeting peptide to the virus particle via a covalent bond (e.g., a disulfide bond). In some embodiments, the PDZ1 protein can be fused to an envelope protein, which can optionally be binding deficient and/or fusion competent virus envelope protein and included in a lentiviral vector. In some embodiments, the TEFCA (SEQ ID NO: 278) can be fused to a cell targeting peptide and the TEFCA-CPT fusion construct can be incorporated into the same or a different lentiviral vector as the PDZ1-envenlope protein construct. During virus production, specific interaction between the PDZ1 and TEFCA (SEQ ID NO: 278) facilitates producing virus particles covalently functionalized with the cell targeting peptide and thus capable of targeting a specific cell-type based upon a specific interaction between the cell targeting peptide and cells expressing its binding partner. This approach can be advantageous for use where surface-incompatibilities can restrict the use of, e.g., cell targeting peptides.

Lentiviral vectors have been disclosed as in the treatment for Parkinson's Disease, see, e.g., US Patent Publication No. 20120295960 and U.S. Pat. Nos. 7,303,910 and 7,351,585. Lentiviral vectors have also been disclosed for the treatment of ocular diseases, see e.g., US Patent Publication Nos. 20060281180, 20090007284, US20110117189; US20090017543; US20070054961, US20100317109. Lentiviral vectors have also been disclosed for delivery to the brain, see, e.g., US Patent Publication Nos. US20110293571; US20110293571, US20040013648, US20070025970, US20090111106 and U.S. Pat. No. 7,259,015. Any of these systems or a variant thereof can be used to deliver an engineered bifunctional receptor encoding polynucleotide described herein to a cell.

In some embodiments, a lentiviral vector system can include one or more transfer plasmids. Transfer plasmids can be generated from various other vector backbones and can include one or more features that can work with other retroviral and/or lentiviral vectors in the system that can, for example, improve safety of the vector and/or vector system, increase virial titers, and/or increase or otherwise enhance expression of the desired insert to be expressed and/or packaged into the viral particle. Suitable features that can be included in a transfer plasmid can include, but are not limited to, 5′LTR, 3′LTR, SIN/LTR, origin of replication (Ori), selectable marker genes (e.g., antibiotic resistance genes), Psi (Ψ), RRE (rev response element), cPPT (central polypurine tract), promoters, WPRE (woodchuck hepatitis post-transcriptional regulatory element), SV40 polyadenylation signal, pUC origin, SV40 origin, F1 origin, and combinations thereof.

In another embodiment, Cocal vesiculovirus envelope pseudotyped retroviral or lentiviral vector particles are contemplated (see, e.g., US Patent Publication No. 20120164118 assigned to the Fred Hutchinson Cancer Research Center). Cocal virus is in the Vesiculovirus genus and is a causative agent of vesicular stomatitis in mammals. Cocal virus was originally isolated from mites in Trinidad (Jonkers et al., Am. J. Vet. Res. 25:236-242 (1964)), and infections have been identified in Trinidad, Brazil, and Argentina from insects, cattle, and horses. Many of the vesiculoviruses that infect mammals have been isolated from naturally infected arthropods, suggesting that they are vector-borne. Antibodies to vesiculoviruses are common among people living in rural areas where the viruses are endemic and laboratory-acquired; infections in humans usually result in influenza-like symptoms. The Cocal virus envelope glycoprotein shares 71.5% identity at the amino acid level with VSV-G Indiana, and phylogenetic comparison of the envelope gene of vesiculoviruses shows that Cocal virus is serologically distinct from, but most closely related to, VSV-G Indiana strains among the vesiculoviruses. Jonkers et al., Am. J. Vet. Res. 25:236-242 (1964) and Travassos da Rosa et al., Am. J. Tropical Med. & Hygiene 33:999-1006 (1984). The Cocal vesiculovirus envelope pseudotyped retroviral vector particles may include for example, lentiviral, alpharetroviral, betaretroviral, gammaretroviral, deltaretroviral, and epsilonretroviral vector particles that may comprise retroviral Gag, Pol, and/or one or more accessory protein(s) and a Cocal vesiculovirus envelope protein. In certain embodiments of these embodiments, the Gag, Pol, and accessory proteins are lentiviral and/or gammaretroviral. In some embodiments, a retroviral vector can contain encoding polypeptides for one or more Cocal vesiculovirus envelope proteins such that the resulting viral or pseudoviral particles are Cocal vesiculovirus envelope pseudotyped.

### Adenoviral Vectors, Helper-Dependent Adenoviral Vectors, and Hybrid Adenoviral Vectors

In some embodiments, the vector can be an adenoviral vector. In some embodiments, the adenoviral vector can include elements such that the virus particle produced using the vector or system thereof can be serotype 2 or serotype 5. In some embodiments, the polynucleotide to be delivered via the adenoviral particle can be up to about 8 kb. Thus, in some embodiments, an adenoviral vector can include a DNA polynucleotide to be delivered that can range in size from about 0.001 kb to about 8 kb. Adenoviral vectors have been used successfully in several contexts (see e.g., Teramato et al. 2000. Lancet. 355:1911-1912; Lai et al. 2002. DNA Cell. Biol. 21:895-913; Flotte et al., 1996. Hum. Gene. Ther. 7:1145-1159; and Kay et al. 2000. Nat. Genet. 24:257-261.

In some embodiments the vector can be a helper-dependent adenoviral vector or system thereof. These are also referred to in the art as “gutless” or “gutted” vectors and are a modified generation of adenoviral vectors (see e.g., Thrasher et al. 2006. Nature. 443:E5-7). In certain embodiments of the helper-dependent adenoviral vector system one vector (the helper) can contain all the viral genes required for replication but contains a conditional gene defect in the packaging domain. The second vector of the system can contain only the ends of the viral genome, one or more engineered bifunctional protein encoding polynucleotides, and the native packaging recognition signal, which can allow selective packaged release from the cells (see e.g., Cideciyan et al. 2009. N Engl J Med. 361:725-727). Helper-dependent adenoviral vector systems have been successful for gene delivery in several contexts (see e.g., Simonelli et al. 2010. J Am Soc Gene Ther. 18:643-650; Cideciyan et al. 2009. N Engl J Med. 361:725-727; Crane et al. 2012. Gene Ther. 19(4):443-452; Alba et al. 2005. Gene Ther. 12:18-S27; Croyle et al. 2005. Gene Ther. 12:579-587; Amalfitano et al. 1998. J. Virol. 72:926-933; and Morral et al. 1999. PNAS. 96:12816-12821). The techniques and vectors described in these publications can be adapted for inclusion and delivery of the engineered bifunctional receptor encoding polynucleotides described herein. In some embodiments, the polynucleotide to be delivered via the viral particle produced from a helper-dependent adenoviral vector or system thereof can be up to about 37 kb. Thus, in some embodiments, an adenoviral vector can include a DNA polynucleotide to be delivered that can range in size from about 0.001 kb to about 37 kb (see e.g., Rosewell et al. 2011. J. Genet. Syndr. Gene Ther. Suppl. 5:001).

In some embodiments, the vector is a hybrid-adenoviral vector or system thereof. Hybrid adenoviral vectors are composed of the high transduction efficiency of a gene-deleted adenoviral vector and the long-term genome-integrating potential of adeno-associated, retroviruses, lentivirus, and transposon based-gene transfer. In some embodiments, such hybrid vector systems can result in stable transduction and limited integration site. See e.g., Balague et al. 2000. Blood. 95:820-828; Morral et al. 1998. Hum. Gene Ther. 9:2709-2716; Kubo and Mitani. 2003. J. Virol. 77(5): 2964-2971; Zhang et al. 2013. PloS One. 8(10) e76771; and Cooney et al. 2015. Mol. Ther. 23(4):667-674), whose techniques and vectors described therein can be modified and adapted for use with the engineered bifunctional receptor encoding polynucleotides of the present invention. In some embodiments, a hybrid-adenoviral vector can include one or more features of a retrovirus and/or an adeno-associated virus. In some embodiments the hybrid-adenoviral vector can include one or more features of a spuma retrovirus or foamy virus (FV). See e.g., Ehrhardt et al. 2007. Mol. Ther. 15:146-156 and Liu et al. 2007. Mol. Ther. 15:1834-1841, whose techniques and vectors described therein can be modified and adapted for use in the CRISPR-Cas system of the present invention. Advantages of using one or more features from the FVs in the hybrid-adenoviral vector or system thereof can include the ability of the viral particles produced therefrom to infect a broad range of cells, a large packaging capacity as compared to other retroviruses, and the ability to persist in quiescent (non-dividing) cells. See also e.g., Ehrhardt et al. 2007. Mol. Ther. 156:146-156 and Shuji et al. 2011. Mol. Ther. 19:76-82, whose techniques and vectors described therein can be modified and adapted for use in the CRISPR-Cas system of the present invention.

### Adeno Associated Viral (AAV) Vectors

In an embodiment, the vector can be an adeno-associated virus (AAV) vector. See, e.g., West et al., Virology 160:38-47 (1987); U.S. Pat. No. 4,797,368; WO 93/24641; Kotin, Human Gene Therapy 5:793-801 (1994); and Muzyczka, J. Clin. Invest. 94:1351 (1994). Although similar to adenoviral vectors in some of their features, AAVs have some deficiency in their replication and/or pathogenicity and thus can be safer than adenoviral vectors. In some embodiments, the AAV can integrate into a specific site on chromosome 19 of a human cell with no observable side effects. In some embodiments, the capacity of the AAV vector, system thereof, and/or AAV particles can be up to about 4.7 kb. The AAV vector or system thereof can include one or more regulatory molecules. In some embodiments the regulatory molecules can be promoters, enhancers, repressors and the like, which are described in greater detail elsewhere herein. In some embodiments, the AAV vector or system thereof can include one or more polynucleotides that can encode one or more regulatory proteins. In some embodiments, the one or more regulatory proteins can be selected from Rep78, Rep68, Rep52, Rep40, variants thereof, and combinations thereof.

The AAV vector or system thereof can include one or more polynucleotides that can encode one or more capsid proteins. The capsid proteins can be selected from VP1, VP2, VP3, and combinations thereof. The capsid proteins can be capable of assembling into a protein shell of the AAV virus particle. In some embodiments, the AAV capsid can contain 60 capsid proteins. In some embodiments, the ratio of VP1:VP2:VP3 in a capsid can be about 1:1:10.

In some embodiments, the AAV vector or system thereof can include one or more adenovirus helper factors or polynucleotides that can encode one or more adenovirus helper factors. Such adenovirus helper factors can include, but are not limited, E1A, E1B, E2A, E40RF6, and VA RNAs. In some embodiments, a producing host cell line expresses one or more of the adenovirus helper factors.

The AAV vector or system thereof can be configured to produce AAV particles having a specific serotype. In some embodiments, the serotype can be AAV-1, AAV-2, AAV-3, AAV-4, AAV-5, AAV-6, AAV-8, AAV-9 or any combinations thereof. In some embodiments, the AAV can be AAV1, AAV-2, AAV-5 or any combination thereof. One can select the AAV of the AAV with regard to the cells to be targeted; e.g., one can select AAV serotypes 1, 2, 5 or a hybrid capsid AAV-1, AAV-2, AAV-5 or any combination thereof for targeting brain and/or neuronal cells; and one can select AAV-4 for targeting cardiac tissue; and one can select AAV8 for delivery to the liver. Thus, in some embodiments, an AAV vector or system thereof capable of producing AAV particles capable of targeting the brain and/or neuronal cells can be configured to generate AAV particles having serotypes 1, 2, 5 or a hybrid capsid AAV-1, AAV-2, AAV-5 or any combination thereof. In some embodiments, an AAV vector or system thereof capable of producing AAV particles capable of targeting cardiac tissue can be configured to generate an AAV particle having an AAV-4 serotype. In some embodiments, an AAV vector or system thereof capable of producing AAV particles capable of targeting the liver can be configured to generate an AAV having an AAV-8 serotype. In some embodiments, the AAV vector is a hybrid AAV vector or system thereof. Hybrid AAVs are AAVs that include genomes with elements from one serotype that are packaged into a capsid derived from at least one different serotype. For example, if it is the rAAV2/5 that is to be produced, and if the production method is based on the helper-free, transient transfection method discussed above, the 1st plasmid and the 3rd plasmid (the adeno helper plasmid) will be the same as discussed for rAAV2 production. However, the second plasmid, the pRepCap will be different. In this plasmid, called pRep2/Cap5, the Rep gene is still derived from AAV2, while the Cap gene is derived from AAV5. The production scheme is the same as the above-mentioned approach for AAV2 production. The resulting rAAV is called rAAV2/5, in which the genome is based on recombinant AAV2, while the capsid is based on AAV5. It is assumed the cell or tissue-tropism displayed by this AAV2/5 hybrid virus should be the same as that of AAV5.

A tabulation of certain AAV serotypes as to these cells can be found in Grimm, D. et al, J. Virol. 82: 5887-5911 (2008).

In some embodiments, the AAV vector or system thereof is configured as a “gutless” vector, similar to that described in connection with a retroviral vector. In some embodiments, the “gutless” AAV vector or system thereof can have the cis-acting viral DNA elements involved in genome amplification and packaging in linkage with the heterologous sequences of interest (e.g., the CRISPR-Cas system polynucleotide(s)).

In some embodiments, the AAV vectors are produced in insect cells, e.g., Spodoptera frugiperda Sf9 insect cells, grown in serum-free suspension culture. Serum-free insect cells can be purchased from commercial vendors, e.g., Sigma Aldrich (EX-CELL 405).

In some embodiments, an AAV vector or vector system can contain or consists essentially of one or more polynucleotides encoding one or more polynucleotides encoding one or more engineered bifunctional receptors.

In another embodiment, an engineered bifunctional receptor polypeptide or encoding polynucleotide is associated with Adeno Associated Virus (AAV), e.g., an AAV comprising an engineered bifunction receptor protein as a fusion, with or without a linker, to or with an AAV capsid protein such as VP1, VP2, and/or VP3. More in particular, modifying the knowledge in the art, e.g., Rybniker et al., “Incorporation of Antigens into Viral Capsids Augments Immunogenicity of Adeno-Associated Virus Vector-Based Vaccines,” J Virol. December 2012; 86(24): 13800-13804, Lux K, et al. 2005. Green fluorescent protein-tagged adeno-associated virus particles allow the study of cytosolic and nuclear trafficking. J. Virol. 79:11776-11787, Munch R C, et al. 2012. “Displaying high-affinity ligands on adeno-associated viral vectors enables tumor cell-specific and safe gene transfer.” Mol. Ther. [Epub ahead of print.] doi:10.1038/mt.2012.186 and Warrington K H, Jr, et al. 2004. Adeno-associated virus type 2 VP2 capsid protein is nonessential and can tolerate large peptide insertions at its N terminus. J. Virol. 78:6595-6609, each incorporated herein by reference, one can obtain a modified AAV capsid of the invention. It will be understood by those skilled in the art that the modifications described herein if inserted into the AAV cap gene may result in modifications in the VP1, VP2 and/or VP3 capsid subunits. Alternatively, the capsid subunits can be expressed independently to achieve modification in only one or two of the capsid subunits (VP1, VP2, VP3, VP1+VP2, VP1+VP3, or VP2+VP3). One can modify the cap gene to have expressed at a desired location a non-capsid protein advantageously a large payload protein, such as an engineered bifunctional receptor protein of the present invention. Likewise, these can be fusions, with the protein, e.g., large payload protein such as an engineered bifunctional receptor protein of the present invention fused in a manner analogous to prior art fusions. See, e.g., US Patent Publication 20090215879; Nance et al., “Perspective on Adeno-Associated Virus Capsid Modification for Duchenne Muscular Dystrophy Gene Therapy,” Hum Gene Ther. 26(12):786-800 (2015) and documents cited therein, incorporated herein by reference. The skilled person, from this disclosure and the knowledge in the art can make and use modified AAV or AAV capsid as in the herein invention, and through this disclosure one knows now that large payload proteins can be fused to the AAV capsid.

Accordingly, the instant invention is also applicable to a virus in the genus Dependoparvovirus or in the family Parvoviridae, for instance, AAV, or a virus of Amdoparvovirus, e.g., Carnivore amdoparvovirus 1, a virus of Aveparvovirus, e.g., Galliform aveparvovirus 1, a virus of Bocaparvovirus, e.g., Ungulate bocaparvovirus 1, a virus of Copiparvovirus, e.g., Ungulate copiparvovirus 1, a virus of Dependoparvovirus, e.g., Adeno-associated dependoparvovirus A, a virus of Erythroparvovirus, e.g., Primate erythroparvovirus 1, a virus of Protoparvovirus, e.g., Rodent protoparvovirus 1, a virus of Tetraparvovirus, e.g., Primate tetraparvovirus 1. Thus, a virus within the family Parvoviridae or the genus Dependoparvovirus or any of the other foregoing genera within Parvoviridae is contemplated as within the invention with discussion herein as to AAV applicable to such other viruses.

In some embodiments, an engineered bifunctional receptor protein of the present invention is external to the capsid or virus particle. In the sense that it is not inside the capsid (enveloped or encompassed with the capsid) but is externally exposed so that it can contact the target polypeptide and/or E3 ligase). In some embodiments, an engineered bifunctional receptor protein of the present invention is associated with the AAV VP2 domain by way of a fusion protein. In some embodiments, the association may be considered to be a modification of the VP2 domain. Where reference is made herein to a modified VP2 domain, then this will be understood to include any association discussed herein of the VP2 domain and the engineered bifunctional receptor protein of the present invention. In some embodiments, the AAV VP2 domain may be associated (or tethered) to the engineered bifunctional receptor protein of the present invention via a connector protein, for example using a system such as the streptavidin-biotin system. In an embodiment, the present invention provides a polynucleotide encoding an engineered bifunctional receptor protein of the present invention and associated AAV VP2 domain. In one embodiment, the invention provides a non-naturally occurring modified AAV having a VP2-engineered bifunctional receptor protein capsid protein, wherein the engineered bifunctional receptor protein is part of or tethered to the VP2 domain. In some preferred embodiments, the engineered bifunctional receptor protein is fused to the VP2 domain so that, in another embodiment, the invention provides a non-naturally occurring modified AAV having a VP2-engineered bifunctional receptor protein fusion capsid protein. Thus, reference herein to a VP2-engineered bifunctional receptor protein may also include a VP2-engineered bifunctional receptor protein fusion capsid protein. In some embodiments, the VP2-engineered bifunctional receptor protein enzyme capsid protein further comprises a linker, whereby the VP2-engineered bifunctional receptor protein is distanced from the remainder of the AAV.

In one embodiment, the invention provides a non-naturally occurring or engineered composition comprising an engineered bifunctional receptor protein which is part of or tethered to an AAV capsid domain, i.e., VP1, VP2, or VP3 domain of Adeno-Associated Virus (AAV) capsid. In some embodiments, part of or tethered to an AAV capsid domain includes associated with a AAV capsid domain. In some embodiments, the engineered bifunctional receptor protein may be fused to the AAV capsid domain. In some embodiments, the fusion may be to the N-terminal end of the AAV capsid domain. As such, in some embodiments, the C-terminal end of the engineered bifunctional receptor protein is fused to the N-terminal end of the AAV capsid domain. In some embodiments, an NLS and/or a linker (such as a GlySer linker) may be positioned between the C-terminal end of the engineered bifunctional receptor protein and the N-terminal end of the AAV capsid domain. In some embodiments, the fusion may be to the C-terminal end of the AAV capsid domain. In some embodiments, this is not preferred due to the fact that the VP1, VP2 and VP3 domains of AAV are alternative splices of the same RNA and so a C-terminal fusion may affect all three domains. In some embodiments, the AAV capsid domain is truncated. In some embodiments, some or all of the AAV capsid domain is removed. In some embodiments, some of the AAV capsid domain is removed and replaced with a linker (such as a GlySer linker), typically leaving the N-terminal and C-terminal ends of the AAV capsid domain intact, such as the first 2, 5 or 10 amino acids. In this way, the internal (non-terminal) portion of the VP3 domain may be replaced with a linker. It is particularly preferred that the linker is fused to the engineered bifunctional receptor protein. A branched linker may be used, with the engineered bifunctional receptor protein fused to the end of one of the branches. This allows for some degree of spatial separation between the capsid and the engineered bifunctional receptor protein. In this way, the engineered bifunctional receptor protein is part of (or fused to) the AAV capsid domain.

In other embodiments, the engineered bifunctional receptor protein may be fused in frame within, i.e., internal to, the AAV capsid domain. Thus, in some embodiments, the AAV capsid domain again preferably retains its N-terminal and C-terminal ends. In this case, a linker is preferred, in some embodiments, either at one or both ends of the engineered bifunctional receptor protein. In this way, the engineered bifunctional receptor protein is again part of (or fused to) the AAV capsid domain. In certain embodiments, the positioning of the engineered bifunctional receptor protein is such that the engineered bifunctional receptor protein is at the external surface of the viral capsid once formed. In one embodiment, the invention provides a non-naturally occurring or engineered composition comprising an engineered bifunctional receptor protein associated with a AAV capsid domain of Adeno-Associated Virus (AAV) capsid. Here, associated may mean in some embodiments fused, or in some embodiments bound to, or in some embodiments tethered to. The engineered bifunctional receptor protein may, in some embodiments, be tethered to the VP1, VP2, or VP3 domain. This may be via a connector protein or tethering system such as the biotin-streptavidin system. In one example, a biotinylation sequence (15 amino acids) could therefore be fused to the engineered bifunctional receptor protein. When a fusion of the AAV capsid domain, especially the N-terminus of the AAV capsid domain, with streptavidin is also provided, the two will therefore associate with very high affinity. Thus, in some embodiments, provided is a composition or system comprising an engineered bifunctional receptor protein-biotin fusion and a streptavidin-AAV capsid domain arrangement, such as a fusion. The engineered bifunctional receptor protein-biotin and streptavidin-AAV capsid domain forms a single complex when the two parts are brought together. NLSs may also be incorporated between the engineered bifunctional receptor protein and the biotin; and/or between the streptavidin and the AAV capsid domain.

As such, provided is a fusion of an engineered bifunctional receptor protein with a connector protein specific for a high affinity ligand for that connector, whereas the AAV VP2 domain is bound to said high affinity ligand. For example, streptavidin may be the connector fused to the engineered bifunctional receptor protein, while biotin may be bound to the AAV VP2 domain. Upon co-localization, the streptavidin will bind to the biotin, thus connecting the engineered bifunctional receptor protein to the AAV VP2 domain. The reverse arrangement is also possible. In some embodiments, a biotinylation sequence (15 amino acids) could therefore be fused to the AAV VP2 domain, especially the N-terminus of the AAV VP2 domain. A fusion of the engineered bifunctional receptor protein with streptavidin is also preferred, in some embodiments. In some embodiments, the biotinylated AAV capsids with streptavidin-engineered bifunctional receptor protein are assembled in vitro. This way the AAV capsids should assemble in a straightforward manner and the engineered bifunctional receptor protein-streptavidin fusion can be added after assembly of the capsid. In other embodiments a biotinylation sequence (15 amino acids) could therefore be fused to the engineered bifunctional receptor protein, together with a fusion of the AAV VP2 domain, especially the N-terminus of the AAV VP2 domain, with streptavidin. For simplicity, a fusion of the engineered bifunctional receptor protein and the AAV VP2 domain is preferred in some embodiments. In some embodiments, the fusion may be to the N-terminal end of the engineered bifunctional receptor protein. In other words, in some embodiments, the AAV and engineered bifunctional receptor protein are associated via fusion. In some embodiments, the AAV and engineered bifunctional receptor protein are associated via fusion including a linker. Suitable linkers are discussed herein and include Gly Ser linkers. Fusion to the N-term of AAV VP2 domain is preferred, in some embodiments. In some embodiments, the engineered bifunctional receptor protein comprises at least one Nuclear Localization Signal (NLS). In a further embodiment, the present invention provides compositions comprising the engineered bifunctional receptor protein and associated AAV VP2 domain or the polynucleotides or vectors described herein. Such compositions and formulations are discussed elsewhere herein.

An alternative tether may be to fuse or otherwise associate the AAV capsid domain to an adaptor protein which binds to or recognizes a corresponding RNA sequence or motif. In some embodiments, the adaptor is or comprises a binding protein which recognizes and binds (or is bound by) an RNA sequence specific for said binding protein. In some embodiments, a preferred example is the MS2 (see Konermann et al. Dec 2014, cited infra, incorporated herein by reference) binding protein which recognizes and binds (or is bound by) an RNA sequence specific for the MS2 protein. With the AAV capsid domain associated with the adaptor protein, the engineered bifunctional receptor protein may, in some embodiments, be tethered to the adaptor protein of the AAV capsid domain.

In certain embodiments, the positioning of the engineered bifunctional receptor protein is such that the engineered bifunctional receptor protein is at the internal surface of the viral capsid once formed. In one embodiment, the invention provides a non-naturally occurring or engineered composition comprising an engineered bifunctional receptor protein associated with an internal surface of an AAV capsid domain. Here again, associated may mean in some embodiments fused, or in some embodiments bound to, or in some embodiments tethered to. The engineered bifunctional receptor protein may, in some embodiments, be tethered to the VP1, VP2, or VP3 domain such that it locates to the internal surface of the viral capsid once formed. This may be via a connector protein or tethering system such as the biotin-streptavidin system as described above and/or elsewhere herein.

In one embodiment, the invention provides a vector system comprising one or more vectors. In some embodiments, two or more vectors comprise one or more engineered bifunctional receptor proteins and/or encoding polynucleotides.

### Herpes Simplex Viral Vectors

In some embodiments, the vector can be a Herpes Simplex Viral (HSV)-based vector or system thereof. HSV systems can include the disabled infections single copy (DISC) viruses, which are composed of a glycoprotein H defective mutant HSV genome. When the defective HSV is propagated in complementing cells, virus particles can be generated that are capable of infecting subsequent cells permanently replicating their own genome but are not capable of producing more infectious particles. See e.g., 2009. Trobridge. Exp. Opin. Biol. Ther. 9:1427-1436, whose techniques and vectors described therein can be modified and adapted for use with the engineered bifunctional receptor protein and/or polynucleotides of the present invention. In some embodiments where an HSV vector or system thereof is utilized, the host cell can be a complementing cell. In some embodiments, HSV vector or system thereof can be capable of producing virus particles capable of delivering a polynucleotide cargo of up to 150 kb. Thus, in some embodiments, the engineered bifunctional receptor protein encoding polynucleotide(s) included in the HSV-based viral vector or system thereof can sum from about 0.001 to about 150 kb. HSV-based vectors and systems thereof have been successfully used in several contexts including various models of neurologic disorders. See e.g., Cockrell et al. 2007. Mol. Biotechnol. 36:184-204; Kafri T. 2004. Mol. Biol. 246:367-390; Balaggan and Ali. 2012. Gene Ther. 19:145-153; Wong et al. 2006. Hum. Gen. Ther. 2002. 17:1-9; Azzouz et al. J. Neruosci. 22L10302-10312; and Betchen and Kaplitt. 2003. Curr. Opin. Neurol. 16:487-493, whose techniques and vectors described therein can be modified and adapted for use in the CRISPR-Cas system of the present invention.

### Poxvirus Vectors

In some embodiments, the vector can be a poxvirus vector or system thereof. In some embodiments, the poxvirus vector can result in cytoplasmic expression of one or more engineered bifunctional receptor protein encoding polynucleotides of the present invention. In some embodiments the capacity of a poxvirus vector or system thereof can be about 25 kb or more. In some embodiments, a poxvirus vector or system thereof can include one or more engineered bifunctional receptor protein encoding polynucleotides described herein.

### Viral Vectors for Delivery to Plants

The systems and compositions may be delivered to plant cells using viral vehicles. In particular embodiments, the compositions and systems may be introduced in the plant cells using a plant viral vector (e.g., as described in Scholthof et al. 1996, Annu Rev Phytopathol. 1996; 34:299-323). Such viral vector may be a vector from a DNA virus, e.g., geminivirus (e.g., cabbage leaf curl virus, bean yellow dwarf virus, wheat dwarf virus, tomato leaf curl virus, maize streak virus, tobacco leaf curl virus, or tomato golden mosaic virus) or nanovirus (e.g., Faba bean necrotic yellow virus). The viral vector may be a vector from an RNA virus, e.g., tobravirus (e.g., tobacco rattle virus, tobacco mosaic virus), potexvirus (e.g., potato virus X), or hordeivirus (e.g., barley stripe mosaic virus). The replicating genomes of plant viruses may be non-integrative vectors.

Virus Particle Production from Viral Vectors

### Retroviral Production

In some embodiments, one or more viral vectors and/or system thereof can be delivered to a suitable cell line for production of virus particles containing the polynucleotide or other payload to be delivered to a host cell. Suitable host cells for virus production from viral vectors and systems thereof described herein are known in the art and are commercially available. For example, suitable host cells include HEK 293 cells and its variants (HEK 293T and HEK 293TN cells). In some embodiments, the suitable host cell for virus production from viral vectors and systems thereof described herein can stably express one or more genes involved in packaging (e.g., pol, gag, and/or VSV-G) and/or other supporting genes.

In some embodiments, after delivery of one or more viral vectors to the suitable host cells for or virus production from viral vectors and systems thereof, the cells are incubated for an appropriate length of time to allow for viral gene expression from the vectors, packaging of the polynucleotide to be delivered (e.g., an engineered bifunctional receptor protein encoding polynucleotide), and virus particle assembly, and secretion of mature virus particles into the culture media. Various other methods and techniques are generally known to those of ordinary skill in the art.

Mature virus particles can be collected from the culture media by a suitable method. In some embodiments, this can involve centrifugation to concentrate the virus. The titer of the composition containing the collected virus particles can be obtained using a suitable method. Such methods can include transducing a suitable cell line (e.g., NIH 3T3 cells) and determining transduction efficiency, infectivity in that cell line by a suitable method. Suitable methods include PCR-based methods, flow cytometry, and antibiotic selection-based methods. Various other methods and techniques are generally known to those of ordinary skill in the art. The concentration of virus particles can be adjusted as needed. In some embodiments, the resulting composition containing virus particles can contain 1×10¹-1×10²⁰ particles/mL.

Lentiviruses may be prepared from any lentiviral vector or vector system described herein. In one example embodiment, after cloning pCasES10 (which contains a lentiviral transfer plasmid backbone), HEK293FT at low passage (p=5) can be seeded in a T-75 flask to 50% confluence the day before transfection in DMEM with 10% fetal bovine serum and without antibiotics. After 20 hours, the media can be changed to OptiMEM (serum-free) media and transfection of the lentiviral vectors can be done 4 hours later. Cells can be transfected with 10 μg of lentiviral transfer plasmid (pCasES10) and the appropriate packaging plasmids (e.g., 5 μg of pMD2.G (VSV-g pseudotype), and 7.5ug of psPAX2 (gag/pol/rev/tat)). Transfection can be carried out in 4 mL OptiMEM with a cationic lipid delivery agent (50 uL Lipofectamine 2000 and 100 ul Plus reagent). After 6 hours, the media can be changed to antibiotic-free DMEM with 10% fetal bovine serum. These methods can use serum during cell culture, but serum-free methods are preferred.

Following transfection and allowing the producing cells (also referred to as packaging cells) to package and produce virus particles with packaged cargo, the lentiviral particles can be purified. In an exemplary embodiment, virus-containing supernatants can be harvested after 48 hours. Collected virus-containing supernatants can first be cleared of debris and filtered through a 0.45 um low protein binding (PVDF) filter. They can then be spun in an ultracentrifuge for 2 hours at 24,000 rpm. The resulting virus-containing pellets can be resuspended in 50 ul of DMEM overnight at 4 degrees C. They can be then aliquoted and used immediately or immediately frozen at −80 degrees C. for storage.

### AAV Particle Production

There are two main strategies for producing AAV particles from AAV vectors and systems thereof, such as those described herein, which depend on how the adenovirus helper factors are provided (helper v. helper free). In some embodiments, a method of producing AAV particles from AAV vectors and systems thereof can include adenovirus infection into cell lines that stably harbor AAV replication and capsid encoding polynucleotides along with AAV vector containing the polynucleotide to be packaged and delivered by the resulting AAV particle (e.g., the engineered bifunctional receptor protein encoding polynucleotide(s)). In some embodiments, a method of producing AAV particles from AAV vectors and systems thereof can be a “helper free” method, which includes co-transfection of an appropriate producing cell line with three vectors (e.g., plasmid vectors): (1) an AAV vector that contains a polynucleotide of interest (e.g., the engineered bifunctional receptor protein encoding polynucleotide(s)) between 2 ITRs; (2) a vector that carries the AAV Rep-Cap encoding polynucleotides; and (helper polynucleotides. One of skill in the art will appreciate various methods and variations thereof that are both helper and —helper free and as well as the different advantages of each system.

### Non-Viral Vectors

In some embodiments, the vector is a non-viral vector or vector system. The term of art “Non-viral vector” and as used herein in this context refers to molecules and/or compositions that are vectors but that are not based on one or more component of a virus or virus genome (excluding any nucleotide to be delivered and/or expressed by the non-viral vector) that can be capable of incorporating engineered bifunctional receptor protein encoding polynucleotide(s) and delivering said engineered bifunctional receptor protein encoding polynucleotide(s) to a cell and/or expressing the polynucleotide in the cell. It will be appreciated that this does not exclude vectors containing a polynucleotide designed to target a virus-based polynucleotide that is to be delivered. For example, if a gRNA or siRNA to be delivered is directed against a virus component and it is inserted or otherwise coupled to an otherwise non-viral vector or carrier, this would not make said vector a “viral vector”. Non-viral vectors can include, without limitation, naked polynucleotides and polynucleotide (non-viral) based vector and vector systems.

### Naked Polynucleotides

In some embodiments one or more engineered bifunctional receptor protein encoding polynucleotides described elsewhere herein can be included in a naked polynucleotide. The term of art “naked polynucleotide” as used herein refers to polynucleotides that are not associated with another molecule (e.g., proteins, lipids, and/or other molecules) that can often help protect it from environmental factors and/or degradation. As used herein, associated with includes, but is not limited to, linked to, adhered to, adsorbed to, enclosed in, enclosed in or within, mixed with, and the like. Naked polynucleotides that include one or more of the engineered bifunctional receptor protein encoding polynucleotides described herein can be delivered directly to a host cell and optionally expressed therein. The naked polynucleotides can have any suitable two- and three-dimensional configurations. By way of non-limiting examples, naked polynucleotides can be single-stranded molecules, double stranded molecules, circular molecules (e.g., plasmids and artificial chromosomes), molecules that contain portions that are single stranded and portions that are double stranded (e.g., ribozymes), and the like. In some embodiments, the naked polynucleotide contains only the engineered bifunctional receptor protein encoding polynucleotide(s) of the present invention. In some embodiments, the naked polynucleotide can contain other nucleic acids and/or polynucleotides in addition to the engineered bifunctional receptor protein encoding polynucleotide(s) of the present invention. The naked polynucleotides can include one or more elements of a transposon system. Transposons and system thereof are described in greater detail elsewhere herein.

### Non-Viral Polynucleotide Vectors

In some embodiments, one or more of the engineered bifunctional receptor protein encoding polynucleotides are included in a non-viral polynucleotide vector. Suitable non-viral polynucleotide vectors include, but are not limited to, transposon vectors and vector systems, plasmids, bacterial artificial chromosomes, yeast artificial chromosomes, AR(antibiotic resistance)-free plasmids and miniplasmids, circular covalently closed vectors (e.g. minicircles, minivectors, miniknots), linear covalently closed vectors (“dumbbell shaped”), MIDGE (minimalistic immunologically defined gene expression) vectors, MiLV (micro-linear vector) vectors, Ministrings, mini-intronic plasmids, PSK systems (post-segregationally killing systems), ORT (operator repressor titration) plasmids, and the like. See e.g., Hardee et al. 2017. Genes. 8(2):65.

In some embodiments, the non-viral polynucleotide vector can have a conditional origin of replication. In some embodiments, the non-viral polynucleotide vector can be an ORT plasmid. In some embodiments, the non-viral polynucleotide vector can have a minimalistic immunologically defined gene expression. In some embodiments, the non-viral polynucleotide vector can have one or more post-segregationally killing system genes. In some embodiments, the non-viral polynucleotide vector is AR-free. In some embodiments, the non-viral polynucleotide vector is a minivector. In some embodiments, the non-viral polynucleotide vector includes a nuclear localization signal. In some embodiments, the non-viral polynucleotide vector can include one or more CpG motifs. In some embodiments, the non-viral polynucleotide vectors can include one or more scaffold/matrix attachment regions (S/MARs). See e.g., Mirkovitch et al. 1984. Cell. 39:223-232, Wong et al. 2015. Adv. Genet. 89:113-152, whose techniques and vectors can be adapted for use in the present invention. S/MARs are AT-rich sequences that play a role in the spatial organization of chromosomes through DNA loop base attachment to the nuclear matrix. S/MARs are often found close to regulatory elements such as promoters, enhancers, and origins of DNA replication. Inclusion of one or S/MARs can facilitate a once-per-cell-cycle replication to maintain the non-viral polynucleotide vector as an episome in daughter cells. In certain embodiments, the S/MAR sequence is located downstream of an actively transcribed polynucleotide (e.g., one or more engineered bifunctional receptor protein encoding polynucleotides of the present invention) included in the non-viral polynucleotide vector. In some embodiments, the S/MAR can be a S/MAR from the beta-interferon gene cluster. See e.g., Verghese et al. 2014. Nucleic Acid Res. 42:e53; Xu et al. 2016. Sci. China Life Sci. 59:1024-1033; Jin et al. 2016. 8:702-711; Koirala et al. 2014. Adv. Exp. Med. Biol. 801:703-709; and Nehlsen et al. 2006. Gene Ther. Mol. Biol. 10:233-244, whose techniques and vectors can be adapted for use in the present invention.

In some embodiments, the non-viral vector is a transposon vector or system thereof. As used herein, “transposon” (also referred to as transposable element) refers to a polynucleotide sequence that is capable of moving from one location in a genome to another. There are several classes of transposons. Transposons include retrotransposons and DNA transposons. Retrotransposons require the transcription of the polynucleotide that is moved (or transposed) in order to transpose the polynucleotide to a new genome or polynucleotide. DNA transposons are those that do not require reverse transcription of the polynucleotide that is moved (or transposed) in order to transpose the polynucleotide to a new genome or polynucleotide. In some embodiments, the non-viral polynucleotide vector can be a retrotransposon vector. In some embodiments, the retrotransposon vector includes long terminal repeats. In some embodiments, the retrotransposon vector does not include long terminal repeats. In some embodiments, the non-viral polynucleotide vector can be a DNA transposon vector. DNA transposon vectors can include a polynucleotide sequence encoding a transposase. In some embodiments, the transposon vector is configured as a non-autonomous transposon vector, meaning that the transposition does not occur spontaneously on its own. In some of these embodiments, the transposon vector lacks one or more polynucleotide sequences encoding proteins required for transposition. In some embodiments, the non-autonomous transposon vectors lack one or more Ac elements.

In some embodiments a non-viral polynucleotide transposon vector system can include a first polynucleotide vector that contains one or more engineered bifunctional receptor protein encoding polynucleotides of the present invention flanked on the 5′ and 3′ ends by transposon terminal inverted repeats (TTRs) and a second polynucleotide vector that includes a polynucleotide capable of encoding a transposase coupled to a promoter to drive expression of the transposase. When both are expressed in the same cell the transposase can be expressed from the second vector and can transpose the material between the TIRs on the first vector (e.g., the engineered bifunctional receptor encoding polynucleotide(s) of the present invention) and integrate it into one or more positions in the host cell's genome. In some embodiments the transposon vector or system thereof can be configured as a gene trap. In some embodiments, the TIRs can be configured to flank a strong splice acceptor site followed by a reporter and/or other gene (e.g., one or more of the engineered bifunctional receptor encoding polynucleotide(s) of the present invention) and a strong poly A tail. When transposition occurs while using this vector or system thereof, the transposon can insert into an intron of a gene and the inserted reporter or other gene can provoke a mis-splicing process and as a result it inactivates the trapped gene.

Any suitable transposon system can be used. Suitable transposon and systems thereof can include Sleeping Beauty transposon system (Tc1/mariner superfamily) (see e.g., Ivics et al. 1997. Cell. 91(4): 501-510), piggyBac (piggyBac superfamily) (see e.g., Li et al. 2013 110(25): E2279-E2287 and Yusa et al. 2011. PNAS. 108(4): 1531-1536), Tol2 (superfamily hAT), Frog Prince (Tc1/mariner superfamily) (see e.g., Miskey et al. 2003 Nucleic Acid Res. 31(23):6873-6881) and variants thereof.

### Non-Vector Delivery Vehicles

The delivery vehicles may comprise non-viral vehicles. In general, methods and vehicles capable of delivering nucleic acids and/or proteins may be used for delivering the systems compositions herein. Examples of non-viral vehicles include lipid nanoparticles, cell-penetrating peptides (CPPs), DNA nanoclews, metal nanoparticles, streptolysin O, multifunctional envelope-type nanodevices (MENDs), lipid-coated mesoporous silica particles, and other inorganic nanoparticles.

### Lipid Particles

The delivery vehicles may comprise lipid particles, e.g., lipid nanoparticles (LNPs) and liposomes. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, International Patent Publication Nos. WO 91/17424 and WO 91/16024. The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

### Lipid Nanoparticles (LNPs)

LNPs may encapsulate nucleic acids within cationic lipid particles (e.g., liposomes), and may be delivered to cells with relative ease. In some examples, lipid nanoparticles do not contain any viral components, which helps minimize safety and immunogenicity concerns. Lipid particles may be used for in vitro, ex vivo, and in vivo deliveries. Lipid particles may be used for various scales of cell populations.

In some examples. LNPs may be used for delivering DNA molecules (e.g., those comprising coding sequences of Cas and/or gRNA) and/or RNA molecules (e.g., mRNA of Cas, gRNAs). In certain cases, LNPs may be use for delivering RNP complexes of Cas/gRNA.

Components in LNPs may comprise cationic lipids 1,2-dilineoyl-3-dimethylammonium-propane (DLinDAP), 1,2-dilinoleyloxy-3-N,N-dimethylaminopropane (DLinDMA), 1,2-dilinoleyloxyketo-N,N-dimethyl-3-aminopropane (DLinK-DMA), 1,2-dilinoleyl-4-(2-dimethylaminoethyl)-[1,3]-dioxolane (DLinKC2-DMA), (3-o-[2″-(methoxypolyethyleneglycol 2000) succinoyl]-1,2-dimyristoyl-sn-glycol (PEG-S-DMG), R-3-[(ro-methoxy-poly(ethylene glycol)2000) carbamoyl]-1,2-dimyristyloxlpropyl-3-amine (PEG-C-DOMG, and any combination thereof. Preparation of LNPs and encapsulation may be adapted from Rosin et al, Molecular Therapy, vol. 19, no. 12, pages 1286-2200, December 2011).

In some embodiments, an LNP delivery vehicle can be used to deliver a virus particle containing an engineered bifunctional receptor encoding polynucleotide and/or polypeptide. In some embodiments, the virus particle(s) can be adsorbed to the lipid particle, such as through electrostatic interactions, and/or can be attached to the liposomes via a linker.

In some embodiments, the LNP contains a nucleic acid, wherein the charge ratio of nucleic acid backbone phosphates to cationic lipid nitrogen atoms is about 1:1.5-7 or about 1:4.

In some embodiments, the LNP also includes a shielding compound, which is removable from the lipid composition under in vivo conditions. In some embodiments, the shielding compound is a biologically inert compound. In some embodiments, the shielding compound does not carry any charge on its surface or on the molecule as such. In some embodiments, the shielding compounds are polyethylenglycoles (PEGs), hydroxyethylglucose (HEG) based polymers, polyhydroxyethyl starch (polyHES) and polypropylene. In some embodiments, the PEG, HEG, polyHES, and a polypropylene weight between about 500 to 10,000 Da or between about 2000 to 5000 Da. In some embodiments, the shielding compound is PEG2000 or PEG5000.

In some embodiments, the LNP can include one or more helper lipids. In some embodiments, the helper lipid can be a phosphor lipid or a steroid. In some embodiments, the helper lipid is between about 20 mol % to 80 mol % of the total lipid content of the composition. In some embodiments, the helper lipid component is between about 35 mol % to 65 mol % of the total lipid content of the LNP. In some embodiments, the LNP includes lipids at 50 mol % and the helper lipid at 50 mol % of the total lipid content of the LNP.

Other non-limiting, exemplary LNP delivery vehicles are described in U.S. Patent Publication Nos. US 20160174546, US 20140301951, US 20150105538, US 20150250725, Wang et al., J. Control Release, 2017 Jan. 31. pii: S0168-3659(17)30038-X. doi: 10.1016/j.jconrel.2017.01.037. [Epub ahead of print]; Altmoğlu et al., Biomater Sci., 4(12):1773-80, Nov. 15, 2016; Wang et al., PNAS, 113(11):2868-73 Mar. 15, 2016; Wang et al., PloS One, 10(11): e0141860. doi: 10.1371/journal.pone.0141860. eCollection 2015, Nov. 3, 2015; Takeda et al., Neural Regen Res. 10(5):689-90, May 2015; Wang et al., Adv. Healthc Mater., 3(9):1398-403, September 2014; and Wang et al., Agnew Chem Int Ed Engl., 53(11):2893-8, Mar. 10, 2014; James E. Dahlman and Carmen Barnes et al. Nature Nanotechnology (2014) published online 11 May 2014, doi:10.1038/nnano.2014.84; Coelho et al., N Engl J Med 2013; 369:819-29; Aleku et al., Cancer Res., 68(23): 9788-98 (Dec. 1, 2008), Strumberg et al., Int. J. Clin. Pharmacol. Ther., 50(1): 76-8 (January 2012), Schultheis et al., J. Clin. Oncol., 32(36): 4141-48 (Dec. 20, 2014), and Fehring et al., Mol. Ther., 22(4): 811-20 (Apr. 22, 2014); Novobrantseva, Molecular Therapy-Nucleic Acids (2012) 1, e4; doi:10.1038/mtna.2011.3; WO2012135025; US 20140348900; US 20140328759; US 20140308304; WO 2005/105152; WO 2006/069782; WO 2007/121947; US 2015/082080; US 20120251618; 7,982,027; 7,799,565; 8,058,069; 8,283,333; 7,901,708; 7,745,651; 7,803,397; 8,101,741; 8,188,263; 7,915,399; 8,236,943 and 7,838,658 and European Pat. Nos 1766035; 1519714; 1781593 and 1664316.

### Liposomes

In some embodiments, a lipid particle may be liposome. Liposomes are spherical vesicle structures composed of a uni- or multilamellar lipid bilayer surrounding internal aqueous compartments and a relatively impermeable outer lipophilic phospholipid bilayer. In some embodiments, liposomes are biocompatible, nontoxic, can deliver both hydrophilic and lipophilic drug molecules, protect their cargo from degradation by plasma enzymes, and transport their load across biological membranes and the blood brain barrier (BBB).

Liposomes can be made from several different types of lipids, e.g., phospholipids. A liposome may comprise natural phospholipids and lipids such as 1,2-distearoryl-sn-glycero-3-phosphatidyl choline (DSPC), DSPE, DSPG, sphingomyelin, egg phosphatidylcholines, monosialoganglioside, or any combination thereof.

Several other additives may be added to liposomes in order to modify their structure and properties. For instance, liposomes may further comprise cholesterol, sphingomyelin, and/or 1,2-dioleoyl-sn-glycero-3-phosphoethanolamine (DOPE), e.g., to increase stability and/or to prevent the leakage of the liposomal inner cargo.

In some embodiments, a liposome delivery vehicle can be used to deliver a virus particle containing an engineered bifunctional receptor encoding polynucleotide and/or polypeptides. In some embodiments, the virus particle(s) can be adsorbed to the liposome, such as through electrostatic interactions, and/or can be attached to the liposomes via a linker.

In some embodiments, the liposome can be a Trojan Horse liposome (also known in the art as Molecular Trojan Horses), see e.g., http://cshprotocols.cshlp.org/content/2010/4/pdb.prot5407.long, the teachings of which can be applied and/or adapted to generated and/or deliver the engineered bifunctional receptor polypeptides and/or encoding polynucleotides.

Other non-limiting, exemplary liposomes can be those as set forth in Wang et al., ACS Synthetic Biology, 1, 403-07 (2012); Wang et al., PNAS, 113(11) 2868-2873 (2016); Spuch and Navarro, Journal of Drug Delivery, vol. 2011, Article ID 469679, 12 pages, 2011. doi:10.1155/2011/469679; WO 2008/042973; U.S. Pat. No. 8,071,082; WO 2014/186366; 20160257951; US20160129120; US 20160244761; 20120251618; WO2013/093648; Lipofectin (a combination of DOTMA and DOPE), Lipofectase, LIPOFECTAMINE.RTM. (e.g., LIPOFECTAMINE.RTM. 2000, LIPOFECTAMINE.RTM. 3000, LIPOFECTAMINE.RTM. RNAiMAX, LIPOFECTAMINE.RTM. LTX), SAINT-RED (Synvolux Therapeutics, Groningen Netherlands), DOPE, Cytofectin (Gilead Sciences, Foster City, Calif), and Eufectins (JBL, San Luis Obispo, Calif.).

### Stable Nucleic-Acid-Lipid Particles (SNALPs)

In some embodiments, the lipid particles may be stable nucleic acid lipid particles (SNALPs). SNALPs may comprise an ionizable lipid (DLinDMA) (e.g., cationic at low pH), a neutral helper lipid, cholesterol, a diffusible polyethylene glycol (PEG)-lipid, or any combination thereof. In some examples, SNALPs may comprise synthetic cholesterol, dipalmitoylphosphatidylcholine, 3-N-[(w-methoxy polyethylene glycol)2000)carbamoyl]-1,2-dimyrestyloxypropylamine, and cationic 1,2-dilinoleyloxy-3-N,Ndimethylaminopropane. In some examples, SNALPs may comprise synthetic cholesterol, 1,2-distearoyl-sn-glycero-3-phosphocholine, PEG-cDMA, and 1,2-dilinoleyloxy-3-(N;N-dimethyl)aminopropane (DLinDMAo).

Other non-limiting, exemplary SNALPs that can be used to deliver the engineered bifunctional receptor polypeptides and/or encoding polynucleotides described herein can be any such SNALPs as described in Morrissey et al., Nature Biotechnology, Vol. 23, No. 8, August 2005, Zimmerman et al., Nature Letters, Vol. 441, 4 May 2006; Geisbert et al., Lancet 2010; 375: 1896-905; Judge, J. Clin. Invest. 119:661-673 (2009); and Semple et al., Nature Niotechnology, Volume 28 Number 2 Feb. 2010, pp. 172-177.

### Other Lipids

The lipid particles may also comprise one or more other types of lipids, e.g., cationic lipids, such as amino lipid 2,2-dilinoleyl-4-dimethylaminoethyl-[1,3]-dioxolane (DLin-KC2-DMA), DLin-KC2-DMA4, C12-200 and colipids disteroylphosphatidyl choline, cholesterol, and PEG-DMG.

In some embodiments, the delivery vehicle can be or include a lipidoid, such as any of those set forth in, for example, US 20110293703.

In some embodiments, the delivery vehicle can be or include an amino lipid, such as any of those set forth in, for example, Jayaraman, Angew. Chem. Int. Ed. 2012, 51, 8529-8533.

In some embodiments, the delivery vehicle can be or include a lipid envelope, such as any of those set forth in, for example, Korman et al., 2011. Nat. Biotech. 29:154-157.

### Lipoplexes Polyplexes

In some embodiments, the delivery vehicles comprise lipoplexes and/or polyplexes. Lipoplexes may bind to negatively charged cell membrane and induce endocytosis into the cells. Examples of lipoplexes may be complexes comprising lipid(s) and non-lipid components. Examples of lipoplexes and polyplexes include FuGENE-6 reagent, a non-liposomal solution containing lipids and other components, zwitterionic amino lipids (ZALs), Ca2b (e.g., forming DNA/Ca²⁺ microcomplexes), polyethenimine (PEI) (e.g., branched PEI), and poly(L-lysine) (PLL).

### Sugar-Based Particles

In some embodiments, the delivery vehicle can be a sugar-based particle. In some embodiments, the sugar-based particles can be or include GalNAc, such as any of those described in WO2014118272; US 20020150626; Nair, J K et al., 2014, Journal of the American Chemical Society 136 (49), 16958-16961; Ostergaard et al., Bioconjugate Chem., 2015, 26 (8), pp 1451-1455.

### Cell Penetrating Peptides

In some embodiments, the delivery vehicles comprise cell penetrating peptides (CPPs). CPPs are short peptides that facilitate cellular uptake of various molecular cargo (e.g., from nanosized particles to small chemical molecules and large fragments of DNA).

CPPs may be of different sizes, amino acid sequences, and charges. In some examples, CPPs can translocate the plasma membrane and facilitate the delivery of various molecular cargoes to the cytoplasm or an organelle. CPPs may be introduced into cells via different mechanisms, e.g., direct penetration in the membrane, endocytosis-mediated entry, and translocation through the formation of a transitory structure.

CPPs may have an amino acid composition that either contains a high relative abundance of positively charged amino acids such as lysine or arginine or has sequences that contain an alternating pattern of polar/charged amino acids and non-polar, hydrophobic amino acids. These two types of structures are referred to as polycationic or amphipathic, respectively. A third class of CPPs are the hydrophobic peptides, containing only apolar residues, with low net charge or have hydrophobic amino acid groups that are crucial for cellular uptake. Another type of CPPs is the trans-activating transcriptional activator (Tat) from Human Immunodeficiency Virus 1 (HIV-1). Examples of CPPs include to Penetratin, Tat (48-60), Transportan, and (R-AhX-R4) (Ahx refers to aminohexanoyl), Kaposi fibroblast growth factor (FGF) signal peptide sequence, integrin β3 signal peptide sequence, polyarginine peptide Args sequence, Guanine rich-molecular transporters, and sweet arrow peptide. Examples of CPPs and related applications also include those described in U.S. Pat. No. 8,372,951.

CPPs can be used for in vitro and ex vivo work quite readily, and extensive optimization for each cargo and cell type is usually required. In some examples, CPPs may be covalently attached to the Cas protein directly, which is then complexed with the gRNA and delivered to cells. In some examples, separate delivery of CPP-Cas and CPP-gRNA to multiple cells may be performed. CPP may also be used to deliver RNPs.

CPPs may be used to deliver the compositions and systems to plants. In some examples, CPPs may be used to deliver the components to plant protoplasts, which are then regenerated to plant cells and further to plants.

### DNA Nanoclews

In some embodiments, the delivery vehicles comprise DNA nanoclews. A DNA nanoclew refers to a sphere-like structure of DNA (e.g., with a shape of a ball of yarn). The nanoclew may be synthesized by rolling circle amplification with palindromic sequences that aide in the self-assembly of the structure. The sphere may then be loaded with a payload. An example of DNA nanoclew is described in Sun W et al, J Am Chem Soc. 2014 Oct. 22; 136(42):14722-5; and Sun W et al, Angew Chem Int Ed Engl. 2015 Oct. 5; 54(41):12029-33. DNA nanoclew may have a palindromic sequence to be partially complementary to the gRNA within the Cas:gRNA ribonucleoprotein complex. A DNA nanoclew may be coated, e.g., coated with PEI to induce endosomal escape.

### Metal Nanoparticles

In some embodiments, the delivery vehicles comprise gold nanoparticles (also referred to AuNPs or colloidal gold). Gold nanoparticles may form complexes with cargos, e.g., Cas:gRNA RNP. Gold nanoparticles may be coated, e.g., coated in a silicate and an endosomal disruptive polymer, PAsp(DET). Examples of gold nanoparticles include AuraSense Therapeutics' Spherical Nucleic Acid (SNA™) constructs, and those described in Mout R, et al. (2017). ACS Nano 11:2452-8; Lee K, et al. (2017). Nat Biomed Eng 1:889-901. Other metal nanoparticles can also be complexed with cargo(s). Such metal particles include tungsten, palladium, rhodium, platinum, and iridium particles. Other non-limiting, exemplary metal nanoparticles are described in US 20100129793.

iTOP

In some embodiments, the delivery vehicles comprise iTOP. iTOP refers to a combination of small molecules that drives the highly efficient intracellular delivery of native proteins, independent of any transduction peptide. iTOP may be used for induced transduction by osmocytosis and propanebetaine, using NaCl-mediated hyperosmolality together with a transduction compound (propanebetaine) to trigger macropinocytotic uptake into cells of extracellular macromolecules. Examples of iTOP methods and reagents include those described in D'Astolfo D S, Pagliero R J, Pras A, et al. (2015). Cell 161:674-690.

### Polymer-Based Particles

In some embodiments, the delivery vehicles may comprise polymer-based particles (e.g., nanoparticles). In some embodiments, the polymer-based particles may mimic a viral mechanism of membrane fusion. The polymer-based particles may be a synthetic copy of Influenza virus machinery and form transfection complexes with various types of nucleic acids (siRNA, miRNA, plasmid DNA or shRNA, mRNA) that cells take up via the endocytosis pathway, a process that involves the formation of an acidic compartment. The low pH in late endosomes acts as a chemical switch that renders the particle surface hydrophobic and facilitates membrane crossing. Once in the cytosol, the particle releases its payload for cellular action. This Active Endosome Escape technology is safe and maximizes transfection efficiency as it is using a natural uptake pathway. In some embodiments, the polymer-based particles may comprise alkylated and carboxyalkylated branched polyethyleneimine. In some examples, the polymer-based particles are VIROMER, e.g., VIROMER RNAi, VIROMER RED, VIROMER mRNA, VIROMER CRISPR. Example methods of delivering the systems and compositions herein include those described in Bawage S S et al., Synthetic mRNA expressed Cas13a mitigates RNA virus infections, www.biorxiv.org/content/10.1101/370460v1.full doi: doi.org/10.1101/370460, Viromer® RED, a powerful tool for transfection of keratinocytes. doi: 10.13140/RG.2.2.16993.61281, Viromer® Transfection—Factbook 2018: technology, product overview, users' data., doi:10.13140/RG.2.2.23912.16642. Other exemplary and non-limiting polymeric particles are described in US 20170079916, US 20160367686, US 20110212179, US 20130302401, 6,007,845, 5,855,913, 5,985,309, 5,543,158, WO2012135025, US 20130252281, US 20130245107, US 20130244279; US 20050019923, 20080267903.

### Streptolysin O (SLO)

The delivery vehicles may be streptolysin 0 (SLO). SLO is a toxin produced by Group A streptococci that works by creating pores in mammalian cell membranes. SLO may act in a reversible manner, which allows for the delivery of proteins (e.g., up to 100 kDa) to the cytosol of cells without compromising overall viability. Examples of SLO include those described in Sierig G, et al. (2003). Infect Immun 71:446-55; Walev I, et al. (2001). Proc Natl Acad Sci USA 98:3185-90; Teng K W, et al. (2017). Elife 6:e25460.

### Multifunctional Envelope-Type Nanodevice (MEND)

The delivery vehicles may comprise multifunctional envelope-type nanodevice (MENDs). MENDs may comprise condensed plasmid DNA, a PLL core, and a lipid film shell. A MEND may further comprise cell-penetrating peptide (e.g., stearyl octaarginine). The cell penetrating peptide may be in the lipid shell. The lipid envelope may be modified with one or more functional components, e.g., one or more of: polyethylene glycol (e.g., to increase vascular circulation time), ligands for targeting of specific tissues/cells, additional cell-penetrating peptides (e.g., for greater cellular delivery), lipids to enhance endosomal escape, and nuclear delivery tags. In some examples, the MEND may be a tetra-lamellar MEND (T-MEND), which may target the cellular nucleus and mitochondria. In certain examples, a MEND may be a PEG-peptide-DOPE-conjugated MEND (PPD-MEND), which may target bladder cancer cells. Examples of MENDs include those described in Kogure K, et al. (2004). J Control Release 98:317-23; Nakamura T, et al. (2012). Acc Chem Res 45:1113-21.

### Lipid-Coated Mesoporous Silica Particles

The delivery vehicles may comprise lipid-coated mesoporous silica particles. Lipid-coated mesoporous silica particles may comprise a mesoporous silica nanoparticle core and a lipid membrane shell. The silica core may have a large internal surface area, leading to high cargo loading capacities. In some embodiments, pore sizes, pore chemistry, and overall particle sizes may be modified for loading different types of cargos. The lipid coating of the particle may also be modified to maximize cargo loading, increase circulation times, and provide precise targeting and cargo release. Examples of lipid-coated mesoporous silica particles include those described in Du X, et al. (2014). Biomaterials 35:5580-90; Durfee P N, et al. (2016). ACS Nano 10:8325-45.

### Inorganic Nanoparticles

The delivery vehicles may comprise inorganic nanoparticles. Examples of inorganic nanoparticles include carbon nanotubes (CNTs) (e.g., as described in Bates K and Kostarelos K. (2013). Adv Drug Deliv Rev 65:2023-33.), bare mesoporous silica nanoparticles (MSNPs) (e.g., as described in Luo G F, et al. (2014). Sci Rep 4:6064), and dense silica nanoparticles (SiNPs) (as described in Luo D and Saltzman W M. (2000). Nat Biotechnol 18:893-5).

### Exosomes

The delivery vehicles may comprise exosomes. Exosomes include membrane bound extracellular vesicles, which can be used to contain and deliver various types of biomolecules, such as proteins, carbohydrates, lipids, and nucleic acids, and complexes thereof (e.g., RNPs). Examples of exosomes include those described in Schroeder A, et al., J Intern Med. 2010 January; 267(1):9-21; E1-Andaloussi S, et al., Nat Protoc. 2012 Dec.; 7(12):2112-26; Uno Y, et al., Hum Gene Ther. 2011 Jun.; 22(6):711-9; Zou W, et al., Hum Gene Ther. 2011 April; 22(4):465-75.

In some examples, the exosome may form a complex (e.g., by binding directly or indirectly) to one or more components of the cargo. In certain examples, a molecule of an exosome may be fused with a first adapter protein and a component of the cargo may be fused with a second adapter protein. The first and the second adapter protein may specifically bind each other, thus associating the cargo with the exosome. Examples of such exosomes include those described in Ye Y, et al., Biomater Sci. 2020 Apr. 28. doi: 10.1039/d0bm00427h.

Other non-limiting, exemplary exosomes include any of those set forth in Alvarez-Erviti et al. 2011, Nat Biotechnol 29: 341; [1401]E1-Andaloussi et al. (Nature Protocols 7:2112-2126(2012); and Wahlgren et al. (Nucleic Acids Research, 2012, Vol. 40, No. 17 e130).

### Spherical Nucleic Acids (SNAs)

In some embodiments, the delivery vehicle can be a SNA. SNAs are three dimensional nanostructures that can be composed of densely functionalized and highly oriented nucleic acids that can be covalently attached to the surface of spherical nanoparticle cores. The core of the spherical nucleic acid can impart the conjugate with specific chemical and physical properties, and it can act as a scaffold for assembling and orienting the oligonucleotides into a dense spherical arrangement that gives rise to many of their functional properties, distinguishing them from all other forms of matter. In some embodiments, the core is a crosslinked polymer. Non-limiting, exemplary SNAs can be any of those set forth in Cutler et al., J. Am. Chem. Soc. 2011 133:9254-9257, Hao et al., Small. 2011 7:3158-3162, Zhang et al., ACS Nano. 2011 5:6962-6970, Cutler et al., J. Am. Chem. Soc. 2012 134:1376-1391, Young et al., Nano Lett. 2012 12:3867-71, Zheng et al., Proc. Natl. Acad. Sci. USA. 2012 109:11975-80, Mirkin, Nanomedicine 2012 7:635-638 Zhang et al., J. Am. Chem. Soc. 2012 134:16488-1691, Weintraub, Nature 2013 495:S14-S16, Choi et al., Proc. Natl. Acad. Sci. USA. 2013 110(19):7625-7630, Jensen et al., Sci. Transl. Med. 5, 209ra152 (2013) and Mirkin, et al., and Small, 10:186-192.

### Self-Assembling Nanoparticles

In some embodiments, the delivery vehicle is a self-assembling nanoparticle. The self-assembling nanoparticles can contain one or more polymers. The self-assembling nanoparticles can be PEGylated. Self-assembling nanoparticles are known in the art. Non-limiting, exemplary self-assembling nanoparticles can be any as set forth in Schiffelers et al., Nucleic Acids Research, 2004, Vol. 32, No. 19, Bartlett et al. (PNAS, Sep. 25, 2007, vol. 104, no. 39; Davis et al., Nature, Vol 464, 15 Apr. 2010.

### Supercharged Proteins

In some embodiments, the delivery vehicle can be a supercharged protein. As used herein “Supercharged proteins” are a class of engineered or naturally occurring proteins with unusually high positive or negative net theoretical charge. Non-limiting, exemplary supercharged proteins can be any of those set forth in Lawrence et al., 2007, Journal of the American Chemical Society 129, 10110-10112.

### Targeted Delivery

In some embodiments, the delivery vehicle can allow for targeted delivery to a specific cell, tissue, organ, or system. In such embodiments, the delivery vehicle can include one or more targeting moieties that can direct targeted delivery of the cargo(s). In an embodiment, the delivery vehicle comprises a targeting moiety, such as active targeting of a lipid entity of the invention, e.g., lipid particle or nanoparticle or liposome or lipid bilayer of the invention comprising a targeting moiety for active targeting.

With regard to targeting moieties, mention is made of Deshpande et al, “Current trends in the use of liposomes for tumor targeting,” Nanomedicine (Lond). 8(9), doi:10.2217/nnm.13.118 (2013), and the documents it cites, all of which are incorporated herein by reference and the teachings of which can be applied and/or adapted for targeted delivery of one or more engineered bifunctional receptor polypeptides and/or encoding polynucleotides described herein. Mention is also made of International Patent Publication No. WO 2016/027264, and the documents it cites, all of which are incorporated herein by reference, the teachings of which can be applied and/or adapted for targeted delivery of one or more engineered bifunctional receptor polypeptides and/or encoding polynucleotides described herein. And mention is made of Lorenzer et al, “Going beyond the liver: Progress and challenges of targeted delivery of siRNA therapeutics,” Journal of Controlled Release, 203: 1-15 (2015), and the documents it cites, all of which are incorporated herein by reference, the teachings of which can be applied and/or adapted for targeted delivery of one or more engineered bifunctional receptor polypeptides and/or encoding polynucleotides described herein.

An actively targeting lipid particle or nanoparticle or liposome or lipid bilayer delivery system (generally as to embodiments of the invention, “lipid entity of the invention” delivery systems) are prepared by conjugating targeting moieties, including small molecule ligands, peptides and monoclonal antibodies, on the lipid or liposomal surface; for example, certain receptors, such as folate and transferrin (Tf) receptors (TfR), are overexpressed on many cancer cells and have been used to make liposomes tumor cell specific. Liposomes that accumulate in the tumor microenvironment can be subsequently endocytosed into the cells by interacting with specific cell surface receptors. To efficiently target liposomes to cells, such as cancer cells, it is useful that the targeting moiety have an affinity for a cell surface receptor and to link the targeting moiety in sufficient quantities to have optimum affinity for the cell surface receptors; and determining these embodiments are within the ambit of the skilled artisan. In the field of active targeting, there are a number of cell-, e.g., tumor-, specific targeting ligands.

Also, as to active targeting, with regard to targeting cell surface receptors such as cancer cell surface receptors, targeting ligands on liposomes can provide attachment of liposomes to cells, e.g., vascular cells, via a noninternalizing epitope; and this can increase the extracellular concentration of that which is being delivered, thereby increasing the amount delivered to the target cells. A strategy to target cell surface receptors, such as cell surface receptors on cancer cells, such as overexpressed cell surface receptors on cancer cells, is to use receptor-specific ligands or antibodies. Many cancer cell types display upregulation of tumor-specific receptors. For example, TfRs and folate receptors (FRs) are greatly overexpressed by many tumor cell types in response to their increased metabolic demand. Folic acid can be used as a targeting ligand for specialized delivery owing to its ease of conjugation to nanocarriers, its high affinity for FRs and the relatively low frequency of FRs, in normal tissues as compared with their overexpression in activated macrophages and cancer cells, e.g., certain ovarian, breast, lung, colon, kidney and brain tumors. Overexpression of FR on macrophages is an indication of inflammatory diseases, such as psoriasis, Crohn's disease, rheumatoid arthritis and atherosclerosis; accordingly, folate-mediated targeting of the invention can also be used for studying, addressing or treating inflammatory disorders, as well as cancers. Folate-linked lipid particles or nanoparticles or liposomes or lipid bylayers of the invention (“lipid entity of the invention”) deliver their cargo intracellularly through receptor-mediated endocytosis. Intracellular trafficking can be directed to acidic compartments that facilitate cargo release, and, most importantly, release of the cargo can be altered or delayed until it reaches the cytoplasm or vicinity of target organelles. Delivery of cargo using a lipid entity of the invention having a targeting moiety, such as a folate-linked lipid entity of the invention, can be superior to nontargeted lipid entity of the invention. The attachment of folate directly to the lipid head groups may not be favorable for intracellular delivery of folate-conjugated lipid entity of the invention, since they may not bind as efficiently to cells as folate attached to the lipid entity of the invention surface by a spacer, which may can enter cancer cells more efficiently. A lipid entity of the invention coupled to folate can be used for the delivery of complexes of lipid, e.g., liposome, e.g., anionic liposome and virus or capsid or envelope or virus outer protein, such as those herein discussed such as adenovirous or AAV. Tf is a monomeric serum glycoprotein of approximately 80 KDa involved in the transport of iron throughout the body. Tf binds to the TfR and translocates into cells via receptor-mediated endocytosis. The expression of TfR can be higher in certain cells, such as tumor cells (as compared with normal cells and is associated with the increased iron demand in rapidly proliferating cancer cells. Accordingly, the invention comprehends a TfR-targeted lipid entity of the invention, e.g., as to liver cells, liver cancer, breast cells such as breast cancer cells, colon such as colon cancer cells, ovarian cells such as ovarian cancer cells, head, neck and lung cells, such as head, neck and non-small-cell lung cancer cells, cells of the mouth such as oral tumor cells.

Also, as to active targeting, a lipid entity of the invention can be multifunctional, i.e., employ more than one targeting moiety such as CPP, along with Tf; a bifunctional system; e.g., a combination of Tf and poly-L-arginine which can provide transport across the endothelium of the blood-brain barrier. EGFR is a tyrosine kinase receptor belonging to the ErbB family of receptors that mediates cell growth, differentiation and repair in cells, especially non-cancerous cells, but EGF is overexpressed in certain cells such as many solid tumors, including colorectal, non-small-cell lung cancer, squamous cell carcinoma of the ovary, kidney, head, pancreas, neck and prostate, and especially breast cancer. The invention comprehends EGFR-targeted monoclonal antibody(ies) linked to a lipid entity of the invention. HER-2 is often overexpressed in patients with breast cancer, and is also associated with lung, bladder, prostate, brain and stomach cancers. HER-2, encoded by the ERBB2 gene. The invention comprehends a HER-2-targeting lipid entity of the invention, e.g., an anti-HER-2-antibody(or binding fragment thereof)-lipid entity of the invention, a HER-2-targeting-PEGylated lipid entity of the invention (e.g., having an anti-HER-2-antibody or binding fragment thereof), a HER-2-targeting-maleimide-PEG polymer-lipid entity of the invention (e.g., having an anti-HER-2-antibody or binding fragment thereof). Upon cellular association, the receptor-antibody complex can be internalized by formation of an endosome for delivery to the cytoplasm.

With respect to receptor-mediated targeting, the skilled artisan takes into consideration ligand/target affinity and the quantity of receptors on the cell surface, and that PEGylation can act as a barrier against interaction with receptors. The use of an antibody-lipid entity of the invention targeting can be advantageous. Multivalent presentation of targeting moieties can also increase the uptake and signaling properties of antibody fragments. In practice of the invention, the skilled person takes into account ligand density (e.g., high ligand densities on a lipid entity of the invention may be advantageous for increased binding to target cells). Preventing early by macrophages can be addressed with a sterically stabilized lipid entity of the invention and linking ligands to the terminus of molecules such as PEG, which is anchored in the lipid entity of the invention (e.g., lipid particle or nanoparticle or liposome or lipid bilayer). The microenvironment of a cell mass such as a tumor microenvironment can be targeted; for instance, it may be advantageous to target cell mass vasculature, such as the tumor vasculature microenvironment. Thus, the invention comprehends targeting VEGF. VEGF and its receptors are well-known proangiogenic molecules and are well-characterized targets for antiangiogenic therapy. Many small-molecule inhibitors of receptor tyrosine kinases, such as VEGFRs or basic FGFRs, have been developed as anticancer agents and the invention comprehends coupling any one or more of these peptides to a lipid entity of the invention, e.g., phage IVO peptide(s) (e.g., via or with a PEG terminus), tumor-homing peptide APRPG (SEQ ID NO: 279) such as APRPG-PEG-modified. VCAM, the vascular endothelium plays a key role in the pathogenesis of inflammation, thrombosis and atherosclerosis. CAMs are involved in inflammatory disorders, including cancer, and are a logical target, E- and P-selectins, VCAM-1 and ICAMs. Can be used to target a lipid entity of the invention., e.g., with PEGylation.

Matrix metalloproteases (MMPs) belong to the family of zinc-dependent endopeptidases. They are involved in tissue remodeling, tumor invasiveness, resistance to apoptosis and metastasis. There are four MMP inhibitors called TIMP1-4, which determine the balance between tumor growth inhibition and metastasis; a protein involved in the angiogenesis of tumor vessels is MT 1-MMP, expressed on newly formed vessels and tumor tissues. The proteolytic activity of MT1-MMP cleaves proteins, such as fibronectin, elastin, collagen and laminin, at the plasma membrane and activates soluble MMPs, such as MMP-2, which degrades the matrix. An antibody or fragment thereof such as a Fab′ fragment can be used in the practice of the invention such as for an antihuman MT1-MMP monoclonal antibody linked to a lipid entity of the invention, e.g., via a spacer such as a PEG spacer. αβ-integrins or integrins are a group of transmembrane glycoprotein receptors that mediate attachment between a cell and its surrounding tissues or extracellular matrix.

Integrins contain two distinct chains (heterodimers) called α- and β-subunits. The tumor tissue-specific expression of integrin receptors can be utilized for targeted delivery in the invention, e.g., whereby the targeting moiety can be an RGD peptide such as a cyclic RGD.

Aptamers are ssDNA or RNA oligonucleotides that impart high affinity and specific recognition of the target molecules by electrostatic interactions, hydrogen bonding and hydrophobic interactions as opposed to the Watson-Crick base pairing, which is typical for the bonding interactions of oligonucleotides. Aptamers as a targeting moiety can have advantages over antibodies: aptamers can demonstrate higher target antigen recognition as compared with antibodies; aptamers can be more stable and smaller in size as compared with antibodies; aptamers can be easily synthesized and chemically modified for molecular conjugation; and aptamers can be changed in sequence for improved selectivity and can be developed to recognize poorly immunogenic targets. Such moieties as a sgc8 aptamer can be used as a targeting moiety (e.g., via covalent linking to the lipid entity of the invention, e.g., via a spacer, such as a PEG spacer).

Also, as to active targeting, the invention also comprehends intracellular delivery. Since liposomes follow the endocytic pathway, they are entrapped in the endosomes (pH 6.5-6) and subsequently fuse with lysosomes (pH<5), where they undergo degradation that results in a lower therapeutic potential. The low endosomal pH can be taken advantage of to escape degradation. Fusogenic lipids or peptides, which destabilize the endosomal membrane after the conformational transition/activation at a lowered pH. Amines are protonated at an acidic pH and cause endosomal swelling and rupture by a buffer effect Unsaturated dioleoylphosphatidylethanolamine (DOPE) readily adopts an inverted hexagonal shape at a low pH, which causes fusion of liposomes to the endosomal membrane. This process destabilizes a lipid entity containing DOPE and releases the cargo into the cytoplasm; fusogenic lipid GALA (SEQ ID NO: 280), cholesteryl-GALA and PEG-GALA may show a highly efficient endosomal release; a pore-forming protein listeriolysin O may provide an endosomal escape mechanism; and, histidine-rich peptides have the ability to fuse with the endosomal membrane, resulting in pore formation, and can buffer the proton pump causing membrane lysis.

The invention comprehends a lipid entity of the invention modified with CPP(s), for intracellular delivery that may proceed via energy dependent macropinocytosis followed by endosomal escape. The invention further comprehends organelle-specific targeting. A lipid entity of the invention surface-functionalized with the triphenylphosphonium (TPP) moiety or a lipid entity of the invention with a lipophilic cation, rhodamine 123 can be effective in delivery of cargo to mitochondria. DOPE/sphingomyelin/stearyl-octa-arginine can deliver cargos to the mitochondrial interior via membrane fusion. A lipid entity of the invention surface modified with a lysosomotropic ligand, octadecyl rhodamine B can deliver cargo to lysosomes. Ceramides are useful in inducing lysosomal membrane permeabilization; the invention comprehends intracellular delivery oaf lipid entity of the invention having a ceramide. The invention further comprehends a lipid entity of the invention targeting the nucleus, e.g., via a DNA-intercalating moiety. The invention also comprehends multifunctional liposomes for targeting, i.e., attaching more than one functional group to the surface of the lipid entity of the invention, for instance to enhances accumulation in a desired site and/or promotes organelle-specific delivery and/or target a particular type of cell and/or respond to the local stimuli such as temperature (e.g., elevated), pH (e.g., decreased), respond to externally applied stimuli such as a magnetic field, light, energy, heat or ultrasound and/or promote intracellular delivery of the cargo. All of these are considered actively targeting moieties.

It should be understood that as to each possible targeting or active targeting moiety herein discussed, there is an embodiment of the invention wherein the delivery system comprises such a targeting or active targeting moiety. Likewise, Table 4 provides a non-exhaustive list of exemplary targeting moieties that can be used in the practice of the invention and as to each an embodiment of the invention provides a delivery system that comprises such a targeting moiety.

TABLE 4

| Targeting Moiety | Target Molecule | Target Cell or Tissue |
|---|---|---|
| folate | folate receptor | cancer cells |
| transferrin | transferrin | cancer cells |
| | receptor | |
| Antibody CC52 | rat CC531 | rat colon |
| | | adenocarcinoma CC531 |
| anti-HER2 antibody | HER2 | HER2-overexpressing |
| | | tumors |
| anti-GD2 | GD2 | neuroblastoma, |
| | | melanoma |
| anti-EGFR | EGFR | tumor cells |
| | | overexpressing EGFR |
| pH-dependent fusogenic | | ovarian carcinoma |
| peptide diINF-7 | | |
| anti-VEGFR | VEGF Receptor | tumor vasculature |
| anti-CD19 | CD19 | leukemia, lymphoma |
| | (B cell marker) | |
| cell-penetrating peptide | | blood-brain barrier |
| cyclic arginine-glycine- | avβ3 | glioblastoma cells, |
| aspartic acid-tyrosine- | | human umbilical vein |
| cysteine (SEQ ID NO: | | endothelial cells, |
| 281) peptide | | tumor angiogenesis |
| (c(RGDyC)-LP) | | |
| ASSHN (SEQ ID NO: | | endothelial progenitor |
| 282) peptide | | cells; anti-cancer |
| PR_b peptide | α₅β₁ integrin | cancer cells |
| AG86 peptide | α₆β₄ integrin | cancer cells |
| KCCYSL (SEQ ID NO: | HER-2 receptor | cancer cells |
| 283) (P6.1 peptide) | | |
| affinity peptide LN | Aminopeptidase N | APN-positive tumor |
| (YEVGHRC (SEQ ID | (APN/CD13) | |
| NO: 284)) | | |
| synthetic somatostatin | Somatostatin | breast cancer |
| analogue | receptor 2 (SSTR2) | |
| anti-CD20 monoclonal | B-lymphocytes | B cell lymphoma |
| antibody | | |

Thus, in an embodiment of the delivery system, the targeting moiety comprises a receptor ligand, such as, for example, hyaluronic acid for CD44 receptor, galactose for hepatocytes, or antibody or fragment thereof such as a binding antibody fragment against a desired surface receptor, and as to each of a targeting moiety comprising a receptor ligand, or an antibody or fragment thereof such as a binding fragment thereof, such as against a desired surface receptor, there is an embodiment of the invention wherein the delivery system comprises a targeting moiety comprising a receptor ligand, or an antibody or fragment thereof such as a binding fragment thereof, such as against a desired surface receptor, or hyaluronic acid for CD44 receptor, galactose for hepatocytes (see, e.g., Surace et al, “Lipoplexes targeting the CD44 hyaluronic acid receptor for efficient transfection of breast cancer cells,” J. Mol Pharm 6(4):1062-73; doi: 10.1021/mp800215d (2009); Sonoke et al, “Galactose-modified cationic liposomes as a liver-targeting delivery system for small interfering RNA,” Biol Pharm Bull. 34(8):1338-42 (2011); Torchilin, “Antibody-modified liposomes for cancer chemotherapy,” Expert Opin. Drug Deliv. 5 (9), 1003-1025 (2008); Manjappa et al, “Antibody derivatization and conjugation strategies: application in preparation of stealth immunoliposome to target chemotherapeutics to tumor,” J. Control. Release 150 (1), 2-22 (2011); Sofou S “Antibody-targeted liposomes in cancer therapy and imaging,” Expert Opin. Drug Deliv. 5 (2): 189-204 (2008); Gao J et al, “Antibody-targeted immunoliposomes for cancer treatment,” Mini. Rev. Med. Chem. 13(14): 2026-2035 (2013); Molavi et al, “Anti-CD30 antibody conjugated liposomal doxorubicin with significantly improved therapeutic efficacy against anaplastic large cell lymphoma,” Biomaterials 34(34):8718-25 (2013), each of which and the documents cited therein are hereby incorporated herein by reference), the teachings of which can be applied and/or adapted for targeted delivery of one or more CRISPR-Cas molecules described herein.

Other exemplary targeting moieties are described elsewhere herein, such as epitope tags and the like.

### Responsive Delivery

In some embodiments, the delivery vehicle can allow for responsive delivery of the cargo(s). Responsive delivery, as used in this context herein, refers to delivery of cargo(s) by the delivery vehicle in response to an external stimuli. Examples of suitable stimuli include, without limitation, an energy (light, heat, cold, and the like), a chemical stimuli (e.g., chemical composition, etc.), and a biologic or physiologic stimuli (e.g., environmental pH, osmolarity, salinity, biologic molecule, etc.). In some embodiments, the targeting moiety can be responsive to an external stimuli and facilitate responsive delivery. In other embodiments, responsiveness is determined by a non-targeting moiety component of the delivery vehicle.

The delivery vehicle can be stimuli-sensitive, e.g., sensitive to an externally applied stimuli, such as magnetic fields, ultrasound or light; and pH-triggering can also be used, e.g., a labile linkage can be used between a hydrophilic moiety such as PEG and a hydrophobic moiety such as a lipid entity of the invention, which is cleaved only upon exposure to the relatively acidic conditions characteristic of the a particular environment or microenvironment such as an endocytic vacuole or the acidotic tumor mass. pH-sensitive copolymers can also be incorporated in embodiments of the invention can provide shielding; diortho esters, vinyl esters, cysteine-cleavable lipopolymers, double esters and hydrazones are a few examples of pH-sensitive bonds that are quite stable at pH 7.5, but are hydrolyzed relatively rapidly at pH 6 and below, e.g., a terminally alkylated copolymer of N-isopropylacrylamide and methacrylic acid that copolymer facilitates destabilization of a lipid entity of the invention and release in compartments with decreased pH value; or, the invention comprehends ionic polymers for generation of a pH-responsive lipid entity of the invention (e.g., poly(methacrylic acid), poly(diethylaminoethyl methacrylate), poly(acrylamide) and poly(acrylic acid)).

Temperature-triggered delivery is also within the ambit of the invention. Many pathological areas, such as inflamed tissues and tumors, show a distinctive hyperthermia compared with normal tissues. Utilizing this hyperthermia is an attractive strategy in cancer therapy since hyperthermia is associated with increased tumor permeability and enhanced uptake. This technique involves local heating of the site to increase microvascular pore size and blood flow, which, in turn, can result in an increased extravasation of embodiments of the invention. Temperature-sensitive lipid entity of the invention can be prepared from thermosensitive lipids or polymers with a low critical solution temperature. Above the low critical solution temperature (e.g., at a site such as tumor site or inflamed tissue site), the polymer precipitates, disrupting the liposomes to release. Lipids with a specific gel-to-liquid phase transition temperature are used to prepare these lipid entities of the invention; and a lipid for a thermosensitive embodiment can be dipalmitoylphosphatidylcholine. Thermosensitive polymers can also facilitate destabilization followed by release, and a useful thermosensitive polymer is poly (N-isopropylacrylamide). Another temperature triggered system can employ lysolipid temperature-sensitive liposomes.

The invention also comprehends redox-triggered delivery. The difference in redox potential between normal and inflamed or tumor tissues, and between the intra- and extra-cellular environments has been exploited for delivery, e.g., GSH is a reducing agent abundant in cells, especially in the cytosol, mitochondria and nucleus. The GSH concentrations in blood and extracellular matrix are just one out of 100 to one out of 1000 of the intracellular concentration, respectively. This high redox potential difference caused by GSH, cysteine and other reducing agents can break the reducible bonds, destabilize a lipid entity of the invention and result in release of payload. The disulfide bond can be used as the cleavable/reversible linker in a lipid entity of the invention, because it causes sensitivity to redox owing to the disulfideto-thiol reduction reaction; a lipid entity of the invention can be made reduction sensitive by using two (e.g., two forms of a disulfide-conjugated multifunctional lipid as cleavage of the disulfide bond (e.g., via tris(2-carboxyethyl)phosphine, dithiothreitol, L-cysteine or GSH), can cause removal of the hydrophilic head group of the conjugate and alter the membrane organization leading to release of payload. Calcein release from a reduction-sensitive lipid entity of the invention containing a disulfide conjugate can be more useful than a reduction-insensitive embodiment.

Enzymes can also be used as a trigger to release payload. Enzymes, including MMPs (e.g., MMP2), phospholipase A2, alkaline phosphatase, transglutaminase or phosphatidylinositol-specific phospholipase C, have been found to be overexpressed in certain tissues, e.g., tumor tissues. In the presence of these enzymes, specially engineered enzyme-sensitive lipid entities of the invention can be disrupted and release the payload. An MMP2-cleavable octapeptide (Gly-Pro-Leu-Gly-Ile-Ala-Gly-Gln (SEQ ID NO: 285)) can be incorporated into a linker, and can have antibody targeting, e.g., antibody 2C5.

The invention also comprehends light- or energy-triggered delivery, e.g., the lipid entity of the invention can be light-sensitive, such that light or energy can facilitate structural and conformational changes, which lead to direct interaction of the lipid entity of the invention with the target cells via membrane fusion, photo-isomerism, photofragmentation or photopolymerization; such a moiety therefor can be benzoporphyrin photosensitizer. Ultrasound can be a form of energy to trigger delivery; a lipid entity of the invention with a small quantity of particular gas, including air or perfluorated hydrocarbon can be triggered to release with ultrasound, e.g., low-frequency ultrasound (LFUS). Magnetic delivery: A lipid entity of the invention can be magnetized by incorporation of magnetites, such as Fe₃O₄ or γ-Fe₂O₃, e.g., those that are less than 10 nm in size. Targeted delivery can be then by exposure to a magnetic field.

### Cells

Described in an example embodiment herein are cell(s) comprising one or more engineered bifunctional receptors of the present invention, engineered bifunctional rector encoding polynucleotides, vector(s), delivery vehicles, and/or the like of the present description, or any combination thereof. Such cells are also referred to herein as “modified cells” as they contain the engineered protein or have been altered by an engineered protein (e.g., have a reduced amount of a target peptide as compared to a suitable control as a result of containing or contained an engineered bifunctional receptor of the present invention). In an example embodiment, the cell is a prokaryotic or eukaryotic cell. In an example embodiment, the cell is a mammalian cell, optionally a human cell. Described in an example embodiment herein are cell populations comprising one or more cells of the present description. As used herein “cell population”, “population of cells”, and the like refers to any number of cells that is more than one and, in some embodiments, refers to 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, 1×10²⁰, 1×10²⁵, 1×10³⁰, 1×10⁴⁰, 1×10⁵⁰ cells or more.

Also described herein are modified cells and progeny thereof that contain engineered bifunctional receptors of the present invention, engineered bifunctional rector encoding polynucleotides, vector(s), delivery vehicles, and/or the like of the present description, or any combination thereof. In some embodiments, the cell is a eukaryotic cell. In some embodiments, the cell is a prokaryotic cell. In some embodiments, the eukaryotic cell is a mammalian cell. In some embodiments the cell is a human cell. In some embodiments, the cell is a non-human mammalian cell. In some embodiments, the cell is a non-human animal cell. In some embodiments, the cell is a plant cell. In some embodiments, the cell is a fungal cell. In some embodiments, the cell is an avian cell. In some embodiments the cell is a fish cell. In some embodiments, the cell is an insect cell. The cells can be modified to contain and/or express the engineered bifunctional receptor of the present invention in vitro, ex vivo, or in vivo. The cells can be modified by delivering a polynucleotide and/or polypeptide of the present invention described in greater detail elsewhere herein or a component thereof into a cell by a suitable delivery mechanism. Suitable delivery methods and techniques include but are not limited to, transfection via a vector, transduction with viral particles, electroporation, endocytic methods, and others, which are described elsewhere herein and will be appreciated by those of ordinary skill in the art in view of this disclosure.

The cells can be further optionally cultured and/or expanded in vitro or ex vivo using any suitable cell culture techniques or conditions, which unless specified otherwise herein, will be appreciated by one of ordinary skill in the art in view of this disclosure. In some embodiments, the cells can be modified, optionally cultured and/or expanded, and/or administered to a subject in need thereof. In some embodiments, cells can be isolated from a subject, subsequently modified and optionally cultured and/or expanded, and administered back to the subject. Such administration can be referred to as autologous administration. In some embodiments, cells can be isolated from a first subject, subsequently modified, optionally cultured and/or expanded, and administered to a second subject, where the first subject and the second subject are different. Such administration can be referred to as non-autologous administration.

The cell can be a neuron, soft tissue (e.g., tendon, muscle, skin, fat, cartilage, etc.), muscle (e.g., cardiac, smooth, skeletal, etc.) cell, bone cell, liver cell, kidney cell, pancreatic cell, stomach cell, intestinal cell, hair follicle, olfactory cell, epithelial cell, heart cell, lung cell, brain cell, blood-brain-barrier cell, or any other cell. In some embodiments, the cell is a stem cell (e.g., an embryonic stem cell, pluripotent stem cell, totipotent stem cell, multipotent stem cell, induced pluripotent stem cell (iPSC), etc.).

In some embodiments the cell is an immune cell or engineered immune cell. The term “immune cell” as used throughout this specification generally encompasses any cell derived from a hematopoietic stem cell that plays a role in the immune response. The term is intended to encompass immune cells both of the innate or adaptive immune system. The immune cell as referred to herein may be a leukocyte, at any stage of differentiation (e.g., a stem cell, a progenitor cell, a mature cell) or any activation stage. Immune cells include lymphocytes (such as natural killer cells, T-cells (including, e.g., thymocytes, Th or Tc; Th1, Th2, Th17, Thαβ, CD4+, CD8+, effector Th, memory Th, regulatory Th, CD4+/CD8+ thymocytes, CD4−/CD8− thymocytes, γδ T cells, etc.) or B-cells (including, e.g., pro-B cells, early pro-B cells, late pro-B cells, pre-B cells, large pre-B cells, small pre-B cells, immature or mature B-cells, producing antibodies of any isotype, T1 B-cells, T2, B-cells, naïve B-cells, GC B-cells, plasmablasts, memory B-cells, plasma cells, follicular B-cells, marginal zone B-cells, B-1 cells, B-2 cells, regulatory B cells, etc.), such as for instance, monocytes (including, e.g., classical, non-classical, or intermediate monocytes), (segmented or banded) neutrophils, eosinophils, basophils, mast cells, histiocytes, microglia, including various subtypes, maturation, differentiation, or activation stages, such as for instance hematopoietic stem cells, myeloid progenitors, lymphoid progenitors, myeloblasts, promyelocytes, myelocytes, metamyelocytes, monoblasts, promonocytes, lymphoblasts, prolymphocytes, small lymphocytes, macrophages (including, e.g., Kupffer cells, stellate macrophages, M1 or M2 macrophages), (myeloid or lymphoid) dendritic cells (including, e.g., Langerhans cells, conventional or myeloid dendritic cells, plasmacytoid dendritic cells, mDC-1, mDC-2, Mo-DC, HP-DC, veiled cells), granulocytes, polymorphonuclear cells, antigen-presenting cells (APC), etc.

In some embodiments, the cells are dysfunctional, exhausted, or are primed to or undergoing transformation. During persistent immune activation, such as during uncontrolled tumor growth or chronic infections, subpopulations of immune cells, particularly of CD8+ or CD4+ T cells, become compromised to different extents with respect to their cytokine and/or cytolytic capabilities. Such immune cells, particularly CD8+ or CD4+ T cells, are commonly referred to as “dysfunctional” or as “functionally exhausted” or “exhausted”. As used herein, the term “dysfunctional” or “functional exhaustion” refer to a state of a cell where the cell does not perform its usual function or activity in response to normal input signals, and includes refractivity of immune cells to stimulation, such as stimulation via an activating receptor or a cytokine. Such a function or activity includes, but is not limited to, proliferation (e.g., in response to a cytokine, such as IFN-gamma) or cell division, entrance into the cell cycle, cytokine production, cytotoxicity, migration and trafficking, phagocytotic activity, or any combination thereof. Normal input signals can include, but are not limited to, stimulation via a receptor (e.g., T cell receptor, B cell receptor, co-stimulatory receptor). Unresponsive immune cells can have a reduction of at least 10%, 20%, 30%, 40%, 50%, 60%, 70%, 80%, 90%, 95%, or even 100% in cytotoxic activity, cytokine production, proliferation, trafficking, phagocytotic activity, or any combination thereof, relative to a corresponding control immune cell of the same type. In some particular embodiments of the aspects described herein, a cell that is dysfunctional is a CD8+ T cell that expresses the CD8+ cell surface marker. Such CD8+ cells normally proliferate and produce cell killing enzymes, e.g., they can release the cytotoxins perforin, granzymes, and granulysin. However, exhausted/dysfunctional T cells do not respond adequately to TCR stimulation, and display poor effector function, sustained expression of inhibitory receptors and a transcriptional state distinct from that of functional effector or memory T cells. Dysfunction/exhaustion of T cells thus prevents optimal control of infection and tumors. Exhausted/dysfunctional immune cells, such as T cells, such as CD8+ T cells, may produce reduced amounts of IFN-gamma, TNF-alpha and/or one or more immunostimulatory cytokines, such as IL-2, compared to functional immune cells. Exhausted/dysfunctional immune cells, such as T cells, such as CD8+ T cells, may further produce (increased amounts of) one or more immunosuppressive transcription factors or cytokines, such as IL-10 and/or Foxp3, compared to functional immune cells, thereby contributing to local immunosuppression. Dysfunctional CD8+ T cells can be both protective and detrimental against disease control. As used herein, a “dysfunctional immune state” refers to an overall suppressive immune state in a subject or microenvironment of the subject (e.g., tumor microenvironment). For example, increased IL-10 production leads to suppression of other immune cells in a population of immune cells.

In some embodiments, the cell is a chimeric antigen receptor (CAR) T cell, T cell receptor cell (TCR), tumor infiltrating lymphocyte (TIL), B-cell, natural killer (NK) cell, or a CAR-NK cell.

In some embodiments, the cells are useful in bioproduction of biologic molecules. In some embodiments, the cells are bioreactors for producing engineered bifunctional receptor polypeptides and/or encoding polynucleotides and/or vectors, delivery vehicles containing the same or as otherwise described herein. In some embodiments, the modified cells produce a bioproduct other than the engineered bifunctional receptors and polynucleotides of the present invention. In such embodiments, the engineered bifunctional receptors target and/or reduce the amount of a target protein whose reduction would improve production of the bioproduct or the bioproduct itself.

### Adoptive Cell Therapies

As used herein, “ACT”, “adoptive cell therapy” and “adoptive cell transfer” may be used interchangeably. In certain embodiments, adoptive cell therapy (ACT) can refer to the transfer of cells to a patient with the goal of transferring the functionality and characteristics into the new host by engraftment of the cells (see, e.g., Mettananda et al., Editing an α-globin enhancer in primary human hematopoietic stem cells as a treatment for β-thalassemia, Nat Commun. 2017 Sep. 4; 8(1):424). As used herein, the term “engraft” or “engraftment” refers to the process of cell incorporation into a tissue of interest in vivo through contact with existing cells of the tissue. Adoptive cell therapy (ACT) can refer to the transfer of cells of any type, most commonly immune-derived cells (e.g., T cells, B cells, natural killer (NK) cells, tumour infiltrating lymphocytes (TILs), etc.) or stem cells (e.g., pluripotent, totipotent, multipotent, induced pluripotent, etc. of varying types (e.g., embryonic, hematopoietic, mesenchymal, adipose-derived, neural, epithelia, etc.), back into the same patient (autologous) or into a new recipient (allogeneic) host with the goal of transferring the functionality and characteristics into the new host, such as immunologic functionality in the case of immune cells or regenerative capacity in the case of stem cells (see e.g., Alessandrini et al. S Afr Med. 2019. 109(10b):70-77; Shin et al., Stem Cell. Rev. Rep. 2020. 16(1):82-93; Cofano et al., Int J Mol Sci. 2019. 20(11):2698; Jang et al., Int J Mol Sci. 2021 22(5):2619; Park et al., Prog Retin Eye Res. 2017 January; 56:148-165; Messina et al., Cells. 2020 Feb. 12; 9(2):420; Stoddard-Bennett. Cells. 2019 Jan. 7; 8(1):26; Qazi et al., J Cachexia Sarcopenia Muscle. 2019 Jun.; 10(3):501-516; Kao et al., Cornea. 2016 November; 35 Suppl 1(Suppl 1):S9-S19; Rosset et al., Orthop Traumatol Surg Res. 2014 February; 100(1 Suppl):S107-12). In some embodiments, such cells are modified prior to returning or delivering to a subject such as to include a chimeric antigen receptor (CAR), an engineered bifunctional receptor of the present invention or polynucleotide or genetic circuit for expressing the same. If possible, use of autologous cells helps the recipient by minimizing GVHD issues. The adoptive transfer of autologous tumor infiltrating lymphocytes (TIL) (Zacharakis et al., (2018) Nat Med. 2018 June; 24(6):724-730; Besser et al., (2010) Clin. Cancer Res 16 (9) 2646-55; Dudley et al., (2002) Science 298 (5594): 850-4; and Dudley et al., (2005) Journal of Clinical Oncology 23 (10): 2346-57.) or genetically re-directed peripheral blood mononuclear cells (Johnson et al., (2009) Blood 114 (3): 535-46; and Morgan et al., (2006) Science 314(5796) 126-9) has been used to successfully treat patients with advanced solid tumors, including melanoma, metastatic breast cancer and colorectal carcinoma, as well as patients with CD19-expressing hematologic malignancies (Kalos et al., (2011) Science Translational Medicine 3 (95): 95ra73). In certain embodiments, allogenic cells immune cells are transferred (see, e.g., Ren et al., (2017) Clin Cancer Res 23 (9) 2255-2266). As described further herein, allogenic cells can be edited or modified to reduce alloreactivity and prevent graft-versus-host disease. Thus, use of allogenic cells allows for cells to be obtained from healthy donors and prepared for use in patients as opposed to preparing autologous cells from a patient after diagnosis.

In some instances, cells involved in cell therapies (and native cell responses) are limited by cell dysfunction (Flemming, A. Nat Rev Immunol. 19:199(2019); Poorebrahim et al., Oncogene. 2021. 40(2):421-435; Thommen and Schumacher et al., Cancer Cell. 2018 Apr. 9; 33(4):547-562; Titov et al., Cancers 2022, 14(4), 1078), exhaustion (see e.g., Blank et al. Nat. Rev. Immunol. 2019. 19(11):665-674; Wherry and Kurachi. Nat. Rev. Immunol. 2015. 15(8):486-499; Fenwick et al., Immunol Rev. 2019 November; 292(1):149-163; Crespo et al., Curr Opin Immunol. 2013 Apr; 25(2):214-21), and/or transformation (Good et al., Cell. 184(25) 2021: 6081-6100; Prockop et al., Mol. Ther. 2010 18(7):1249-1250; Sato et al., Cytotherapy. 2019. 21(11):1095-1111) that can hinder the efficacy of the cell therapy or lead to undesired malignancies or other issues. It will be appreciated that such dysfunctions and transformations are not limited to cells used in cell therapies but also exist in native cells, such as native immune cells, involved in fighting infection (e.g., viral or bacterial infection) or disease (e.g., cancer). Thus, although discussed herein the context of adoptive cell therapies, the engineered bifunctional receptors can be used in native cells in similar ways to combat dysfunction, exhaustion, and or transformation that may exist in the native cells.

Aspects of the invention involve the adoptive transfer of immune system cells, such as T cells or NK cells, specific for selected antigens, such as tumor associated antigens or tumor specific neoantigens (see, e.g., Maus et al., 2014, Adoptive Immunotherapy for Cancer or Viruses, Annual Review of Immunology, Vol. 32: 189-225; Rosenberg and Restifo, 2015, Adoptive cell transfer as personalized immunotherapy for human cancer, Science Vol. 348 no. 6230 pp. 62-68; Restifo et al., 2015, Adoptive immunotherapy for cancer: harnessing the T cell response. Nat. Rev. Immunol. 12(4): 269-281; and Jenson and Riddell, 2014, Design and implementation of adoptive therapy with chimeric antigen receptor-modified T cells. Immunol Rev. 257(1): 127-144; and Rajasagi et al., 2014, Systematic identification of personal tumor-specific neoantigens in chronic lymphocytic leukemia. Blood. 2014 Jul. 17; 124(3):453-62).

In certain embodiments, an antigen (such as a tumor antigen or self-antigen) to be targeted in adoptive cell therapy (such as particularly CAR or TCR T-cell therapy) of a disease (such as particularly of tumor or cancer) may be selected from a group consisting of: MR1 (see, e.g., Crowther, et al., 2020, Genome-wide CRISPR-Cas9 screening reveals ubiquitous T cell cancer targeting via the monomorphic MHC class I-related protein MR1, Nature Immunology volume 21, pages 178-185), B cell maturation antigen (BCMA) (see, e.g., Friedman et al., Effective Targeting of Multiple BCMA-Expressing Hematological Malignancies by Anti-BCMA CAR T Cells, Hum Gene Ther. 2018 Mar. 8; Berdeja J G, et al. Durable clinical responses in heavily pretreated patients with relapsed/refractory multiple myeloma: updated results from a multicenter study of bb2121 anti-Bcma CAR T cell therapy. Blood. 2017; 130:740; and Mouhieddine and Ghobrial, Immunotherapy in Multiple Myeloma: The Era of CAR T Cell Therapy, Hematologist, May-June 2018, Volume 15, issue 3); PSA (prostate-specific antigen); prostate-specific membrane antigen (PSMA); PSCA (Prostate stem cell antigen); Tyrosine-protein kinase transmembrane receptor ROR1; fibroblast activation protein (FAP); Tumor-associated glycoprotein 72 (TAG72); Carcinoembryonic antigen (CEA); Epithelial cell adhesion molecule (EPCAM); Mesothelin; Human Epidermal growth factor Receptor 2 (ERBB2 (Her2/neu)); Prostase; Prostatic acid phosphatase (PAP); elongation factor 2 mutant (ELF2M); Insulin-like growth factor 1 receptor (IGF-1R); gplOO; BCR-ABL (breakpoint cluster region-Abelson); tyrosinase; New York esophageal squamous cell carcinoma 1 (NY-ESO-1); κ-light chain, LAGE (L antigen); MAGE (melanoma antigen); Melanoma-associated antigen 1 (MAGE-A1); MAGE A3; MAGE A6; legumain; Human papillomavirus (HPV) E6; HPV E7; prostein; survivin; PCTA1 (Galectin 8); Melan-A/MART-1; Ras mutant; TRP-1 (tyrosinase related protein 1, or gp75); Tyrosinase-related Protein 2 (TRP2); TRP-2/INT2 (TRP-2/intron 2); RAGE (renal antigen); receptor for advanced glycation end products 1 (RAGE1); Renal ubiquitous 1, 2 (RU1, RU2); intestinal carboxyl esterase (iCE); Heat shock protein 70-2 (HSP70-2) mutant; thyroid stimulating hormone receptor (TSHR); CD123; CD171; CD19; CD20; CD22; CD26; CD30; CD33; CD44v7/8 (cluster of differentiation 44, exons 7/8); CD53; CD92; CD100; CD148; CD150; CD200; CD261; CD262; CD362; CS-1 (CD2 subset 1, CRACC, SLAMF7, CD319, and 19A24); C-type lectin-like molecule-1 (CLL-1); ganglioside GD3 (aNeu5Ac(2-8)aNeu5Ac(2-3)bDGalp(1-4)bDGlcp(1-1)Cer); Tn antigen (Tn Ag); Fms-Like Tyrosine Kinase 3 (FLT3); CD38; CD138; CD44v6; B7H3 (CD276); KIT (CD 117); Interleukin-13 receptor subunit alpha-2 (IL-13Ra2); Interleukin 11 receptor alpha (IL-11Ra); prostate stem cell antigen (PSCA); Protease Serine 21 (PRSS21); vascular endothelial growth factor receptor 2 (VEGFR2); Lewis(Y) antigen; CD24; Platelet-derived growth factor receptor beta (PDGFR-beta); stage-specific embryonic antigen-4 (SSEA-4); Mucin 1, cell surface associated (MUC1); mucin 16 (MUC16); epidermal growth factor receptor (EGFR); epidermal growth factor receptor variant III (EGFRvIII); neural cell adhesion molecule (NCAM); carbonic anhydrase IX (CAIX); Proteasome (Prosome, Macropain) Subunit, Beta Type, 9 (LMP2); ephrin type-A receptor 2 (EphA2); Ephrin B2; Fucosyl GM1; sialyl Lewis adhesion molecule (sLe); ganglioside GM3 (aNeu5Ac(2-3)bDGalp(1-4)bDGlcp(1-1)Cer); TGS5; high molecular weight-melanoma-associated antigen (HMWMAA); o-acetyl-GD2 ganglioside (OAcGD2); Folate receptor alpha; Folate receptor beta; tumor endothelial marker 1 (TEM1/CD248); tumor endothelial marker 7-related (TEM7R); claudin 6 (CLDN6); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); CT (cancer/testis (antigen)); melanoma cancer testis antigen-1 (MAD-CT-1); melanoma cancer testis antigen-2 (MAD-CT-2); Fos-related antigen 1; p53; p53 mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin Bi; Cyclin D1; v-myc avian myelocytomatosis viral oncogene neuroblastoma derived homolog (MYCN); Ras Homolog Family Member C (RhoC); Cytochrome P450 1B1 (CYP1B1); CCCTC-Binding Factor (Zinc Finger Protein)-Like (BORIS); Squamous Cell Carcinoma Antigen Recognized By T Cells-1 or 3 (SART1, SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint-1, -2, -3 or -4 (SSX1, SSX2, SSX3, SSX4); CD79a; CD79b; CD72; Leukocyte-associated immunoglobulin-like receptor 1 (LAIR1); Fc fragment of IgA receptor (FCAR); Leukocyte immunoglobulin-like receptor subfamily A member 2 (LILRA2); CD300 molecule-like family member f (CD300LF); C-type lectin domain family 12 member A (CLECi2A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); mouse double minute 2 homolog (MDM2); livin; alphafetoprotein (AFP); transmembrane activator and CAML Interactor (TACI); B-cell activating factor receptor (BAFF-R); V-Ki-ras2 Kirsten rat sarcoma viral oncogene homolog (KRAS); immunoglobulin lambda-like polypeptide 1 (IGLL1); 707-AP (707 alanine proline); ART-4 (adenocarcinoma antigen recognized by T4 cells); BAGE (B antigen; b-catenin/m, b-catenin/mutated); CAMEL (CTL-recognized antigen on melanoma); CAP1 (carcinoembryonic antigen peptide 1); CASP-8 (caspase-8); CDC27m (cell-division cycle 27 mutated); CDK4/m (cycline-dependent kinase 4 mutated); Cyp-B (cyclophilin B); DAM (differentiation antigen melanoma); EGP-2 (epithelial glycoprotein 2); EGP-40 (epithelial glycoprotein 40); Erbb2, 3, 4 (erythroblastic leukemia viral oncogene homolog-2, -3, 4); FBP (folate binding protein); fAchR (Fetal acetylcholine receptor); G250 (glycoprotein 250); GAGE (G antigen); GnT-V (N-acetylglucosaminyltransferase V); HAGE (helicose antigen); ULA-A (human leukocyte antigen-A); HST2 (human signet ring tumor 2); KIAA0205; KDR (kinase insert domain receptor); LDLR/FUT (low density lipid receptor/GDP L-fucose: b-D-galactosidase 2-a-L fucosyltransferase); L1CAM (L1 cell adhesion molecule); MC1R (melanocortin 1 receptor); Myosin/m (myosin mutated); MUM-1, -2, -3 (melanoma ubiquitous mutated 1, 2, 3); NA88-A (NA cDNA clone of patient M88); KG2D (Natural killer group 2, member D) ligands; oncofetal antigen (h5T4); p190 minor bcr-abl (protein of 190KD bcr-abl); Pml/RARa (promyelocytic leukemia/retinoic acid receptor a); PRAME (preferentially expressed antigen of melanoma); SAGE (sarcoma antigen); TEL/AML1 (translocation Ets-family leukemia/acute myeloid leukemia 1); TPI/m (triosephosphate isomerase mutated); CD70; and any combination thereof.

In certain embodiments, an antigen to be targeted in adoptive cell therapy (such as CAR or TCR T-cell therapy) of a disease (such as particularly of tumor or cancer) is a tumor-specific antigen (TSA).

In certain embodiments, an antigen to be targeted in adoptive cell therapy (such as particularly CAR or TCR T-cell therapy) of a disease (such as particularly of tumor or cancer) is a neoantigen.

In certain embodiments, an antigen to be targeted in adoptive cell therapy (such as particularly CAR or TCR T-cell therapy) of a disease (such as particularly of tumor or cancer) is a tumor-associated antigen (TAA).

In certain embodiments, an antigen to be targeted in adoptive cell therapy (such as particularly CAR or TCR T-cell therapy) of a disease (such as particularly of tumor or cancer) is a universal tumor antigen. In certain preferred embodiments, the universal tumor antigen is selected from the group consisting of: a human telomerase reverse transcriptase (hTERT), survivin, mouse double minute 2 homolog (MDM2), cytochrome P450 1B 1 (CYP1B), HER2/neu, Wilms' tumor gene 1 (WT1), livin, alphafetoprotein (AFP), carcinoembryonic antigen (CEA), mucin 16 (MUC16), MUC1, prostate-specific membrane antigen (PSMA), p53, cyclin (D1), and any combinations thereof.

In certain embodiments, an antigen (such as a tumor antigen) to be targeted in adoptive cell therapy (such as particularly CAR or TCR T-cell therapy) of a disease (such as particularly of tumor or cancer) may be selected from a group consisting of. CD19, BCMA, CD70, CLL-1, MAGE A3, MAGE A6, HPV E6, HPV E7, WT1, CD22, CD171, ROR1, MUC16, and SSX2. In certain preferred embodiments, the antigen may be CD19. For example, CD19 may be targeted in hematologic malignancies, such as in lymphomas, more particularly in B-cell lymphomas, such as without limitation in diffuse large B-cell lymphoma, primary mediastinal b-cell lymphoma, transformed follicular lymphoma, marginal zone lymphoma, mantle cell lymphoma, acute lymphoblastic leukemia including adult and pediatric ALL, non-Hodgkin lymphoma, indolent non-Hodgkin lymphoma, or chronic lymphocytic leukemia. For example, BCMA may be targeted in multiple myeloma or plasma cell leukemia (see, e.g., 2018 American Association for Cancer Research (AACR) Annual meeting Poster: Allogeneic Chimeric Antigen Receptor T Cells Targeting B Cell Maturation Antigen). For example, CLL1 may be targeted in acute myeloid leukemia. For example, MAGE A3, MAGE A6, SSX2, and/or KRAS may be targeted in solid tumors. For example, HPV E6 and/or HPV E7 may be targeted in cervical cancer or head and neck cancer. For example, WT1 may be targeted in acute myeloid leukemia (AML), myelodysplastic syndromes (MDS), chronic myeloid leukemia (CML), non-small cell lung cancer, breast, pancreatic, ovarian or colorectal cancers, or mesothelioma. For example, CD22 may be targeted in B cell malignancies, including non-Hodgkin lymphoma, diffuse large B-cell lymphoma, or acute lymphoblastic leukemia. For example, CD171 may be targeted in neuroblastoma, glioblastoma, or lung, pancreatic, or ovarian cancers. For example, ROR1 may be targeted in ROR1+ malignancies, including non-small cell lung cancer, triple negative breast cancer, pancreatic cancer, prostate cancer, ALL, chronic lymphocytic leukemia, or mantle cell lymphoma. For example, MUC16 may be targeted in MUC16ecto+ epithelial ovarian, fallopian tube or primary peritoneal cancer. For example, CD70 may be targeted in both hematologic malignancies as well as in solid cancers such as renal cell carcinoma (RCC), gliomas (e.g., GBM), and head and neck cancers (HNSCC). CD70 is expressed in both hematologic malignancies as well as in solid cancers, while its expression in normal tissues is restricted to a subset of lymphoid cell types (see, e.g., 2018 American Association for Cancer Research (AACR) Annual meeting Poster: Allogeneic CRISPR Engineered Anti-CD70 CAR-T Cells Demonstrate Potent Preclinical Activity Against Both Solid and Hematological Cancer Cells).

Various strategies may for example be employed to genetically modify T cells by altering the specificity of the T cell receptor (TCR) for example by introducing new TCR a and R chains with selected peptide specificity (see U.S. Pat. No. 8,697,854; PCT Patent Publications: WO2003020763, WO2004033685, WO2004044004, WO2005114215, WO2006000830, WO2008038002, WO2008039818, WO2004074322, WO2005113595, WO2006125962, WO2013166321, WO2013039889, WO2014018863, WO2014083173; U.S. Pat. No. 8,088,379).

As an alternative to, or addition to, TCR modifications, chimeric antigen receptors (CARs) may be used in order to generate immunoresponsive cells, such as T cells or natural killer cells (NK), specific for selected targets, such as malignant cells, with a wide variety of receptor chimera constructs having been described (see U.S. Pat. Nos. 5,843,728; 5,851,828; 5,912,170; 6,004,811; 6,284,240; 6,392,013; 6,410,014; 6,753,162; 8,211,422; and, PCT Publication WO9215322).

In general, CARs are comprised of an extracellular domain, a transmembrane domain, and an intracellular domain, wherein the extracellular domain comprises an antigen-binding domain that is specific for a predetermined target (see, e.g., Gong Y, Klein Wolterink R G J, Wang J, Bos G M J, Germeraad W T V. Chimeric antigen receptor natural killer (CAR-NK) cell design and engineering for cancer therapy. J Hematol Oncol. 2021; 14(1):73; Guedan S, Calderon H, Posey A D Jr, Maus M V. Engineering and Design of Chimeric Antigen Receptors. Mol Ther Methods Clin Dev. 2018; 12:145-156; Petersen C T, Krenciute G. Next Generation CAR T Cells for the Immunotherapy of High-Grade Glioma. Front Oncol. 2019; 9:69; and Lu H, Zhao X, Li Z, Hu Y, Wang H. From CAR-T Cells to CAR-NK Cells: A Developing Immunotherapy Method for Hematological Malignancies. Front Oncol. 2021). While the antigen-binding domain of a CAR is often an antibody or antibody fragment (e.g., a single chain variable fragment, scFv), the binding domain is not particularly limited so long as it results in specific recognition of a target. For example, in some embodiments, the antigen-binding domain may comprise a receptor, such that the CAR is capable of binding to the ligand of the receptor. Alternatively, the antigen-binding domain may comprise a ligand, such that the CAR is capable of binding the endogenous receptor of that ligand.

The antigen-binding domain of a CAR is generally separated from the transmembrane domain by a hinge or spacer. The spacer is also not particularly limited, and it is designed to provide the CAR with flexibility. For example, a spacer domain may comprise a portion of a human Fc domain, including a portion of the CH3 domain, or the hinge region of any immunoglobulin, such as IgA, IgD, IgE, IgG, or IgM, or variants thereof. Furthermore, the hinge region may be modified so as to prevent off-target binding by FcRs or other potential interfering objects. For example, the hinge may comprise an IgG4 Fc domain with or without a S228P, L235E, and/or N297Q mutation (according to Kabat numbering) in order to decrease binding to FcRs. Additional spacers/hinges include, but are not limited to, CD4, CD8, and CD28 hinge regions.

The transmembrane domain of a CAR may be derived either from a natural or from a synthetic source. Where the source is natural, the domain may be derived from any membrane bound or transmembrane protein. Transmembrane regions of particular use in this disclosure may be derived from CD8, CD28, CD3, CD45, CD4, CD5, CDS, CD9, CD 16, CD22, CD33, CD37, CD64, CD80, CD86, CD 134, CD137, CD 154, TCR. Alternatively, the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

Alternative CAR constructs may be characterized as belonging to successive generations. First-generation CARs typically consist of a single-chain variable fragment of an antibody specific for an antigen, for example comprising a VL linked to a VH of a specific antibody, linked by a flexible linker, for example by a CD8a hinge domain and a CD8a transmembrane domain, to the transmembrane and intracellular signaling domains of either CD3ζ or FcRγ (scFv-CD3ζ or scFv-FcRγ; see U.S. Pat. Nos. 7,741,465; 5,912,172; U.S. Pat. No. 5,906,936). Second-generation CARs incorporate the intracellular domains of one or more costimulatory molecules, such as CD28, OX40 (CD134), or 4-1BB (CD137) within the endodomain (for example scFv-CD28/OX40/4-1BB-CD3ζ; see U.S. Pat. Nos. 8,911,993; 8,916,381; 8,975,071; 9,101,584; 9,102,760; 9,102,761). Third-generation CARs include a combination of costimulatory endodomains, such a CD3ζ-chain, CD97, GDI 1a-CD18, CD2, ICOS, CD27, CD154, CDS, OX40, 4-1BB, CD2, CD7, LIGHT, LFA-1, NKG2C, B7-H3, CD30, CD40, PD-1, or CD28 signaling domains (for example scFv-CD28-4-1BB-CD3ζ or scFv-CD28-OX40-CD3ζ; see U.S. Pat. Nos. 8,906,682; 8,399,645; 5,686,281; PCT Publication No. WO2014134165; PCT Publication No. WO2012079000). In certain embodiments, the primary signaling domain comprises a functional signaling domain of a protein selected from the group consisting of CD3 zeta, CD3 gamma, CD3 delta, CD3 epsilon, common FcR gamma (FCERIG), FcR beta (Fc Epsilon Rib), CD79a, CD79b, Fc gamma RIIa, DAP10, and DAP12. In certain preferred embodiments, the primary signaling domain comprises a functional signaling domain of CD3ζ or FcRγ. In certain embodiments, the one or more costimulatory signaling domains comprise a functional signaling domain of a protein selected, each independently, from the group consisting of: CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8 alpha, CD8 beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAMI, CRTAM, Ly9 (CD229), CD160 (BY55), PSGL1, CD100 (SEMA4D), CD69, SLAMF6 (NTB-A, Lyl08), SLAM (SLAMFI, CD150, IPO-3), BLAME (SLAMF8), SELPLG (CD162), LTBR, LAT, GADS, SLP-76, PAG/Cbp, NKp44, NKp30, NKp46, and NKG2D. In certain embodiments, the one or more costimulatory signaling domains comprise a functional signaling domain of a protein selected, each independently, from the group consisting of: 4-1BB, CD27, and CD28. In certain embodiments, a chimeric antigen receptor may have the design as described in U.S. Pat. No. 7,446,190, comprising an intracellular domain of CD3ζ chain (such as amino acid residues 52-163 of the human CD3 zeta chain, as shown in SEQ ID NO: 14 of U.S. Pat. No. 7,446,190), a signaling region from CD28 and an antigen-binding element (or portion or domain; such as scFv). The CD28 portion, when between the zeta chain portion and the antigen-binding element, may suitably include the transmembrane and signaling domains of CD28 (such as amino acid residues 114-220 of SEQ ID NO: 10, full sequence shown in SEQ ID NO: 6 of U.S. Pat. No. 7,446,190; these can include the following portion of CD28 as set forth in Genbank identifier NM_006139 (sequence version 1, 2 or 3): IEVMYPPPYLDNEKSNGTIIHVKGKHLCPSPLFPGPSKPFWVLVVVGGVLACYSLLVTVA FIIFWVRSKRSRLLHSDYMNMTPRRPGPTRKHYQPYAPPRDFAAYRS)) (SEQ. I.D. No. 286). Alternatively, when the zeta sequence lies between the CD28 sequence and the antigen-binding element, intracellular domain of CD28 can be used alone (such as amino sequence set forth in SEQ ID NO: 9 of U.S. Pat. No. 7,446,190). Hence, certain embodiments employ a CAR comprising (a) a zeta chain portion comprising the intracellular domain of human CD3ζ chain, (b) a costimulatory signaling region, and (c) an antigen-binding element (or portion or domain), wherein the costimulatory signaling region comprises the amino acid sequence encoded by SEQ ID NO: 6 of U.S. Pat. No. 7,446,190.

Alternatively, costimulation may be orchestrated by expressing CARs in antigen-specific T cells, chosen so as to be activated and expanded following engagement of their native αβTCR, for example by antigen on professional antigen-presenting cells, with attendant costimulation. In addition, additional engineered receptors may be provided on the immunoresponsive cells, for example to improve targeting of a T-cell attack and/or minimize side effects

By means of an example and without limitation, Kochenderfer et al., (2009) J Immunother. 32 (7): 689-702 described anti-CD19 chimeric antigen receptors (CAR). FMC63-28Z CAR contained a single chain variable region moiety (scFv) recognizing CD19 derived from the FMC63 mouse hybridoma (described in Nicholson et al., (1997) Molecular Immunology 34: 1157-1165), a portion of the human CD28 molecule, and the intracellular component of the human TCR-(molecule. FMC63-CD828BBZ CAR contained the FMC63 scFv, the hinge and transmembrane regions of the CD8 molecule, the cytoplasmic portions of CD28 and 4-1BB, and the cytoplasmic component of the TCR-(molecule. The exact sequence of the CD28 molecule included in the FMC63-28Z CAR corresponded to Genbank identifier NM_006139; the sequence included all amino acids starting with the amino acid sequence IEVMYPPPY (SEQ. I.D. No. 287) and continuing all the way to the carboxy-terminus of the protein. To encode the anti-CD19 scFv component of the vector, the authors designed a DNA sequence which was based on a portion of a previously published CAR (Cooper et al., (2003) Blood 101: 1637-1644). This sequence encoded the following components in frame from the 5′ end to the 3′ end: an XhoI site, the human granulocyte-macrophage colony-stimulating factor (GM-CSF) receptor α-chain signal sequence, the FMC63 light chain variable region (as in Nicholson et al., supra), a linker peptide (as in Cooper et al., supra), the FMC63 heavy chain variable region (as in Nicholson et al., supra), and a NotI site. A plasmid encoding this sequence was digested with XhoI and NotI. To form the MSGV-FMC63-28Z retroviral vector, the XhoI and NotI-digested fragment encoding the FMC63 scFv was ligated into a second XhoI and NotI-digested fragment that encoded the MSGV retroviral backbone (as in Hughes et al., (2005) Human Gene Therapy 16: 457-472) as well as part of the extracellular portion of human CD28, the entire transmembrane and cytoplasmic portion of human CD28, and the cytoplasmic portion of the human TCR-(molecule (as in Maher et al., 2002) Nature Biotechnology 20: 70-75). The FMC63-28Z CAR is included in the KTE-C19 (axicabtagene ciloleucel) anti-CD19 CAR-T therapy product in development by Kite Pharma, Inc. for the treatment of inter alia patients with relapsed/refractory aggressive B-cell non-Hodgkin lymphoma (NHL). Accordingly, in certain embodiments, cells intended for adoptive cell therapies, more particularly immunoresponsive cells such as T cells, may express the FMC63-28Z CAR as described by Kochenderfer et al. (supra). Hence, in certain embodiments, cells intended for adoptive cell therapies, more particularly immunoresponsive cells such as T cells, may comprise a CAR comprising an extracellular antigen-binding element (or portion or domain; such as scFv) that specifically binds to an antigen, an intracellular signaling domain comprising an intracellular domain of a CD3ζ chain, and a costimulatory signaling region comprising a signaling domain of CD28. Preferably, the CD28 amino acid sequence is as set forth in Genbank identifier NM_006139 (sequence version 1, 2 or 3) starting with the amino acid sequence IEVMYPPPY (SEQ ID NO: 287) and continuing all the way to the carboxy-terminus of the protein. The sequence is reproduced herein:

(SEQ ID NO: 286)
IEVMYPPPYLDNEKSNGTIIHVKGKHLCPSPLFPGPSKPFWVLVVVGGV
LACYSLLVTVAFIIFWVRSKRSRLLHSDYMNMTPRRPGPTRKHYQPYAP
PRDFAAYRS

Preferably, the antigen is CD19, more preferably the antigen-binding element is an anti-CD19 scFv, even more preferably the anti-CD19 scFv as described by Kochenderfer et al. (supra).

Additional anti-CD19 CARs are further described in WO2015187528. More particularly Example 1 and Table 1 of WO2015187528, incorporated by reference herein, demonstrate the generation of anti-CD19 CARs based on a fully human anti-CD19 monoclonal antibody (47G4, as described in US20100104509) and murine anti-CD19 monoclonal antibody (as described in Nicholson et al. and explained above). Various combinations of a signal sequence (human CD8-alpha or GM-CSF receptor), extracellular and transmembrane regions (human CD8-alpha) and intracellular T-cell signaling domains (CD28-CD3ζ; 4-1BB-CD3ζ; CD27-CD3ζ; CD28-CD27-CD3ζ, 4-1BB-CD27-CD3ζ; CD27-4-1BB-CD3ζ; CD28-CD27-FcΣRI gamma chain; or CD28-FcΣRI gamma chain) were disclosed. Hence, in certain embodiments, cells intended for adoptive cell therapies, more particularly immunoresponsive cells such as T cells, may comprise a CAR comprising an extracellular antigen-binding element that specifically binds to an antigen, an extracellular and transmembrane region as set forth in Table 1 of WO2015187528 and an intracellular T-cell signaling domain as set forth in Table 1 of WO2015187528. Preferably, the antigen is CD19, more preferably the antigen-binding element is an anti-CD19 scFv, even more preferably the mouse or human anti-CD19 scFv as described in Example 1 of WO2015187528. In certain embodiments, the CAR comprises, consists essentially of or consists of an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13 as set forth in Table 1 of WO2015187528.

By means of an example and without limitation, chimeric antigen receptor that recognizes the CD70 antigen is described in WO2012058460A2 (see also, Park et al., CD70 as a target for chimeric antigen receptor T cells in head and neck squamous cell carcinoma, Oral Oncol. 2018 March; 78:145-150; and Jin et al., CD70, a novel target of CAR T-cell therapy for gliomas, Neuro Oncol. 2018 Jan. 10; 20(1):55-65). CD70 is expressed by diffuse large B-cell and follicular lymphoma and also by the malignant cells of Hodgkin's lymphoma, Waldenstrom's macroglobulinemia and multiple myeloma, and by HTLV-1- and EBV-associated malignancies. (Agathanggelou et al. Am. J. Pathol. 1995; 147: 1152-1160; Hunter et al., Blood 2004; 104:4881. 26; Lens et al., J Immunol. 2005; 174:6212-6219; Baba et al., J Virol. 2008; 82:3843-3852.) In addition, CD70 is expressed by non-hematological malignancies such as renal cell carcinoma and glioblastoma. (Junker et al., J Urol. 2005; 173:2150-2153; Chahlavi et al., Cancer Res 2005; 65:5428-5438) Physiologically, CD70 expression is transient and restricted to a subset of highly activated T, B, and dendritic cells.

By means of an example and without limitation, chimeric antigen receptor that recognizes BCMA has been described (see, e.g., US20160046724A1; WO2016014789A2; WO2017211900A1; WO2015158671A1; US20180085444A1; WO2018028647A1; US20170283504A1; and WO2013154760A1).

In certain embodiments, the immune cell may, in addition to a CAR or exogenous TCR as described herein, further comprise a chimeric inhibitory receptor (inhibitory CAR) that specifically binds to a second target antigen and is capable of inducing an inhibitory or immunosuppressive or repressive signal to the cell upon recognition of the second target antigen. In certain embodiments, the chimeric inhibitory receptor comprises an extracellular antigen-binding element (or portion or domain) configured to specifically bind to a target antigen, a transmembrane domain, and an intracellular immunosuppressive or repressive signaling domain. In certain embodiments, the second target antigen is an antigen that is not expressed on the surface of a cancer cell or infected cell or the expression of which is downregulated on a cancer cell or an infected cell. In certain embodiments, the second target antigen is an MHC-class I molecule. In certain embodiments, the intracellular signaling domain comprises a functional signaling portion of an immune checkpoint molecule, such as for example PD-1 or CTLA4. Advantageously, the inclusion of such inhibitory CAR reduces the chance of the engineered immune cells attacking non-target (e.g., non-cancer) tissues.

Alternatively, T-cells expressing CARs may be further modified to reduce or eliminate expression of endogenous TCRs in order to reduce off-target effects. Reduction or elimination of endogenous TCRs can reduce off-target effects and increase the effectiveness of the T cells (U.S. Pat. No. 9,181,527). T cells stably lacking expression of a functional TCR may be produced using a variety of approaches. T cells internalize, sort, and degrade the entire T cell receptor as a complex, with a half-life of about 10 hours in resting T cells and 3 hours in stimulated T cells (von Essen, M. et al. 2004. J. Immunol. 173:384-393). Proper functioning of the TCR complex requires the proper stoichiometric ratio of the proteins that compose the TCR complex. TCR function also requires two functioning TCR zeta proteins with ITAM motifs. The activation of the TCR upon engagement of its MHC-peptide ligand requires the engagement of several TCRs on the same T cell, which all must signal properly. Thus, if a TCR complex is destabilized with proteins that do not associate properly or cannot signal optimally, the T cell will not become activated sufficiently to begin a cellular response.

Accordingly, in some embodiments, TCR expression may eliminated using RNA interference (e.g., shRNA, siRNA, miRNA, etc.), CRISPR, or other methods that target the nucleic acids encoding specific TCRs (e.g., TCR-α and TCR-β) and/or CD3 chains in primary T cells. By blocking expression of one or more of these proteins, the T cell will no longer produce one or more of the key components of the TCR complex, thereby destabilizing the TCR complex and preventing cell surface expression of a functional TCR.

In some instances, CAR may also comprise a switch mechanism for controlling expression and/or activation of the CAR. For example, a CAR may comprise an extracellular, transmembrane, and intracellular domain, in which the extracellular domain comprises a target-specific binding element that comprises a label, binding domain, or tag that is specific for a molecule other than the target antigen that is expressed on or by a target cell. In such embodiments, the specificity of the CAR is provided by a second construct that comprises a target antigen binding domain (e.g., an scFv or a bispecific antibody that is specific for both the target antigen and the label or tag on the CAR) and a domain that is recognized by or binds to the label, binding domain, or tag on the CAR. See, e.g., WO 2013/044225, WO 2016/000304, WO 2015/057834, WO 2015/057852, WO 2016/070061, U.S. Pat. No. 9,233,125, US 2016/0129109. In this way, a T-cell that expresses the CAR can be administered to a subject, but the CAR cannot bind its target antigen until the second composition comprising an antigen-specific binding domain is administered.

Alternative switch mechanisms include CARs that require multimerization in order to activate their signaling function (see, e.g., US 2015/0368342, US 2016/0175359, US 2015/0368360) and/or an exogenous signal, such as a small molecule drug (US 2016/0166613, Yung et al., Science, 2015), in order to elicit a T-cell response. Some CARs may also comprise a “suicide switch” to induce cell death of the CAR T-cells following treatment (Buddee et al., PLoS One, 2013) or to downregulate expression of the CAR following binding to the target antigen (WO 2016/011210).

Alternative techniques may be used to transform target immunoresponsive cells, such as protoplast fusion, lipofection, transfection or electroporation. A wide variety of vectors may be used, such as retroviral vectors, lentiviral vectors, adenoviral vectors, adeno-associated viral vectors, plasmids or transposons, such as a Sleeping Beauty transposon (see U.S. Pat. Nos. 6,489,458; 7,148,203; 7,160,682; 7,985,739; 8,227,432), may be used to introduce CARs, for example using 2nd generation antigen-specific CARs signaling through CD3ζ and either CD28 or CD137. Viral vectors may for example include vectors based on HIV, SV40, EBV, HSV or BPV. In certain embodiments, inducible gene switches are used to regulate expression of a CAR or TCR (see, e.g., Chakravarti, Deboki et al. “Inducible Gene Switches with Memory in Human T Cells for Cellular Immunotherapy.” ACS synthetic biology vol. 8,8 (2019): 1744-1754).

Cells that are targeted for transformation may for example include T cells, Natural Killer (NK) cells, cytotoxic T lymphocytes (CTL), regulatory T cells, human embryonic stem cells, tumor-infiltrating lymphocytes (TIL) or a pluripotent stem cell from which lymphoid cells may be differentiated. T cells expressing a desired CAR may for example be selected through co-culture with γ-irradiated activating and propagating cells (AaPC), which co-express the cancer antigen and co-stimulatory molecules. The engineered CAR T-cells may be expanded, for example by co-culture on AaPC in presence of soluble factors, such as IL-2 and IL-21. This expansion may for example be carried out so as to provide memory CAR+ T cells (which may for example be assayed by non-enzymatic digital array and/or multi-panel flow cytometry). In this way, CAR T cells may be provided that have specific cytotoxic activity against antigen-bearing tumors (optionally in conjunction with production of desired chemokines such as interferon-γ). CAR T cells of this kind may for example be used in animal models, for example to treat tumor xenografts.

In certain embodiments, ACT includes co-transferring CD4+ Th1 cells and CD8+ CTLs to induce a synergistic antitumour response (see, e.g., Li et al., Adoptive cell therapy with CD4+ T helper 1 cells and CD8+ cytotoxic T cells enhances complete rejection of an established tumour, leading to generation of endogenous memory responses to non-targeted tumour epitopes. Clin Transl Immunology. 2017 October; 6(10): e160).

In certain embodiments, antigen specificity can be conferred to Tregs by engineering the expression of transgenic T-cell receptor (TCR) or chimeric antigen receptor (CAR), such as to modulate immune responses in organ transplant and autoimmune diseases (see, e.g., Arjomandnejad M, Kopec A L, Keeler A M. CAR-T Regulatory (CAR-Treg) Cells: Engineering and Applications. Biomedicines. 2022; 10(2):287). Regulatory T cells (Tregs) are a T-cell subset known for their immunomodulatory function. Expression of CD4, CD25, and the master transcription factor, forkhead box P3 (FOXP3), are the main characteristic markers of conventional Tregs. However, other regulatory immune cells with different properties such as CD8+ Tregs, or type 1 regulatory T cells (Tr1) have been described. Id. Tregs are divided into “natural” Tregs that develop in the thymus or “induced” Tregs that are generated in the periphery. Id. Regulatory T cells suppress immune responses through multiple mechanisms including direct interaction with other immune cells or by producing immunosuppressive cytokines such as interleukin-10 (IL-10) and Transforming growth factor beta (TGF-β). Id. Directing Tregs towards a desired antigen may boost the overall response and lower the risk of broad and systemic immunosuppression or generation of an inflammatory response. Id.

In certain embodiments, Th17 cells are transferred to a subject in need thereof. Th17 cells have been reported to directly eradicate melanoma tumors in mice to a greater extent than Th1 cells (Muranski P, et al., Tumor-specific Th17-polarized cells eradicate large established melanoma. Blood. 2008 Jul. 15; 112(2):362-73; and Martin-Orozco N, et al., T helper 17 cells promote cytotoxic T cell activation in tumor immunity. Immunity. 2009 Nov. 20; 31(5):787-98). Those studies involved an adoptive T cell transfer (ACT) therapy approach, which takes advantage of CD4+ T cells that express a TCR recognizing tyrosinase tumor antigen. Exploitation of the TCR leads to rapid expansion of Th17 populations to large numbers ex vivo for reinfusion into the autologous tumor-bearing hosts.

In certain embodiments, ACT may include autologous iPSC-based vaccines, such as irradiated iPSCs in autologous anti-tumor vaccines (see e.g., Kooreman, Nigel G. et al., Autologous iPSC-Based Vaccines Elicit Anti-tumor Responses In Vivo, Cell Stem Cell 22, 1-13, 2018, doi.org/10.1016/j.stem.2018.01.016).

Unlike T-cell receptors (TCRs) that are MHC restricted, CARs can potentially bind any cell surface-expressed antigen and can thus be more universally used to treat patients (see Irving et al., Engineering Chimeric Antigen Receptor T-Cells for Racing in Solid Tumors: Don't Forget the Fuel, Front. Immunol., 3 Apr. 2017, doi.org/10.3389/fimmu.2017.00267). In certain embodiments, in the absence of endogenous T-cell infiltrate (e.g., due to aberrant antigen processing and presentation), which precludes the use of TIL therapy and immune checkpoint blockade, the transfer of CAR T-cells may be used to treat patients (see, e.g., Hinrichs C S, Rosenberg S A. Exploiting the curative potential of adoptive T-cell therapy for cancer. Immunol Rev (2014) 257(1):56-71. doi:10.1111/imr.12132).

Approaches such as the foregoing may be adapted to provide methods of treating and/or increasing survival of a subject having a disease, such as a neoplasia, for example by administering an effective amount of an immunoresponsive cell comprising an antigen recognizing receptor that binds a selected antigen, wherein the binding activates the immunoresponsive cell, thereby treating or preventing the disease (such as a neoplasia, a pathogen infection, an autoimmune disorder, or an allogeneic transplant reaction).

In certain embodiments, the treatment can be administered after lymphodepleting pretreatment in the form of chemotherapy (typically a combination of cyclophosphamide and fludarabine) or radiation therapy. Initial studies in ACT had short lived responses and the transferred cells did not persist in vivo for very long (Houot et al., T-cell-based immunotherapy: adoptive cell transfer and checkpoint inhibition. Cancer Immunol Res (2015) 3(10):1115-22; and Kamta et al., Advancing Cancer Therapy with Present and Emerging Immuno-Oncology Approaches. Front. Oncol. (2017) 7:64). Immune suppressor cells like Tregs and MDSCs may attenuate the activity of transferred cells by outcompeting them for the necessary cytokines. Not being bound by a theory lymphodepleting pretreatment may eliminate the suppressor cells allowing the TILs to persist.

In one embodiment, the treatment can be administered into patients undergoing an immunosuppressive treatment (e.g., glucocorticoid treatment). The cells, or population of cells, may be made resistant to at least one immunosuppressive agent due to the inactivation of a gene encoding a receptor for such immunosuppressive agent. In certain embodiments, the immunosuppressive treatment provides for the selection and expansion of the immunoresponsive T cells within the patient.

In certain embodiments, the treatment can be administered before primary treatment (e.g., surgery or radiation therapy) to shrink a tumor before the primary treatment. In another embodiment, the treatment can be administered after primary treatment to remove any remaining cancer cells.

In certain embodiments, immunometabolic barriers can be targeted therapeutically prior to and/or during ACT to enhance responses to ACT or CAR T-cell therapy and to support endogenous immunity (see, e.g., Irving et al., Engineering Chimeric Antigen Receptor T-Cells for Racing in Solid Tumors: Don't Forget the Fuel, Front. Immunol., 3 Apr. 2017, doi.org/10.3389/fimmu.2017.00267).

The administration of cells or population of cells, such as immune system cells or cell populations, such as more particularly immunoresponsive cells or cell populations, as disclosed herein may be carried out in any convenient manner, including by aerosol inhalation, injection, ingestion, transfusion, implantation or transplantation. The cells or population of cells may be administered to a patient subcutaneously, intradermally, intratumorally, intranodally, intramedullary, intramuscularly, intrathecally, by intravenous or intralymphatic injection, or intraperitoneally. In some embodiments, the disclosed CARs may be delivered or administered into a cavity formed by the resection of tumor tissue (i.e., intracavity delivery) or directly into a tumor prior to resection (i.e., intratumoral delivery). In one embodiment, the cell compositions of the present invention are preferably administered by intravenous injection.

The administration of the cells or population of cells can consist of the administration of 10⁴-10⁹ cells per kg body weight, preferably 10⁵ to 10⁶ cells/kg body weight including all integer values of cell numbers within those ranges. Dosing in CAR T cell therapies may for example involve administration of from 10⁶ to 10⁹ cells/kg, with or without a course of lymphodepletion, for example with cyclophosphamide. The cells or population of cells can be administered in one or more doses. In another embodiment, the effective amount of cells are administered as a single dose. In another embodiment, the effective amount of cells are administered as more than one dose over a period time. Timing of administration is within the judgment of a managing physician and depends on the clinical condition of the patient. The cells or population of cells may be obtained from any source, such as a blood bank or a donor. While individual needs vary, determination of optimal ranges of effective amounts of a given cell type for a particular disease or conditions are within the skill of one in the art. An effective amount means an amount which provides a therapeutic or prophylactic benefit. The dosage administered will be dependent upon the age, health and weight of the recipient, kind of concurrent treatment, if any, frequency of treatment and the nature of the effect desired.

In another embodiment, the effective amount of cells or composition comprising those cells are administered parenterally. The administration can be an intravenous administration. The administration can be directly done by injection within a tumor.

To guard against possible adverse reactions, engineered immunoresponsive cells may be equipped with a transgenic safety switch, in the form of a transgene that renders the cells vulnerable to exposure to a specific signal. For example, the herpes simplex viral thymidine kinase (TK) gene may be used in this way, for example by introduction into allogeneic T lymphocytes used as donor lymphocyte infusions following stem cell transplantation (Greco, et al., Improving the safety of cell therapy with the TK-suicide gene. Front. Pharmacol. 2015; 6: 95). In such cells, administration of a nucleoside prodrug such as ganciclovir or acyclovir causes cell death. Alternative safety switch constructs include inducible caspase 9, for example triggered by administration of a small-molecule dimerizer that brings together two nonfunctional icasp9 molecules to form the active enzyme. A wide variety of alternative approaches to implementing cellular proliferation controls have been described (see U.S. Patent Publication No. 20130071414; PCT Patent Publication WO2011146862; PCT Patent Publication WO2014011987; PCT Patent Publication WO2013040371; Zhou et al. BLOOD, 2014, 123/25:3895-3905; Di Stasi et al., The New England Journal of Medicine 2011; 365:1673-1683; Sadelain M, The New England Journal of Medicine 2011; 365:1735-173; Ramos et al., Stem Cells 28(6):1107-15 (2010)).

In a further refinement of adoptive therapies, immunoresponsive cells can be modified and tailored by any suitable genetic modification system or method (e.g., gene editing by programmable nucleases) to alternative implementations, for example providing edited CAR T cells (see Poirot et al., 2015, Multiplex genome edited T-cell manufacturing platform for “off-the-shelf” adoptive T-cell immunotherapies, Cancer Res 75 (18): 3853; Ren et al., 2017, Multiplex genome editing to generate universal CAR T cells resistant to PD1 inhibition, Clin Cancer Res. 2017 May 1; 23(9):2255-2266. doi: 10.1158/1078-0432.CCR-16-1300. Epub 2016 Nov 4; Qasim et al., 2017, Molecular remission of infant B-ALL after infusion of universal TALEN gene-edited CAR T cells, Sci Transl Med. 2017 Jan. 25; 9(374); Legut, et al., 2018, CRISPR-mediated TCR replacement generates superior anticancer transgenic T cells. Blood, 131(3), 311-322; Georgiadis et al., Long Terminal Repeat CRISPR-CAR-Coupled “Universal” T Cells Mediate Potent Anti-leukemic Effects, Molecular Therapy, In Press, Corrected Proof, Available online 6 Mar. 2018; and Roth, T. L. Editing of Endogenous Genes in Cellular Immunotherapies. Curr Hematol Malig Rep 15, 235-240 (2020)). In some embodiments, cells may be edited using any programmable nuclease system or other suitable system (e.g., a CRISPR-Cas system, Zinc-Finger nuclease, Iscbm Omega, or other system). Such systems may be delivered to an immune cell by any method described herein. In preferred embodiments, cells are modified ex vivo and transferred to a subject in need thereof. Immunoresponsive cells, CAR T cells or any cells used for adoptive cell transfer may be edited or otherwise modified. Editing or other modification may be performed for example to insert or knock-in an exogenous gene, such as an exogenous gene encoding a CAR or a TCR, at a preselected locus in a cell (e.g., TRAC locus); to eliminate potential alloreactive T-cell receptors (TCR) or to prevent inappropriate pairing between endogenous and exogenous TCR chains, such as to knock-out or knock-down expression of an endogenous TCR in a cell; to disrupt the target of a chemotherapeutic agent in a cell; to block an immune checkpoint, such as to knock-out or knock-down expression of an immune checkpoint protein or receptor in a cell; to knock-out or knock-down expression of other gene or genes in a cell, the reduced expression or lack of expression of which can enhance the efficacy of adoptive therapies using the cell; to knock-out or knock-down expression of an endogenous gene in a cell, said endogenous gene encoding an antigen targeted by an exogenous CAR or TCR; to knock-out or knock-down expression of one or more MHC constituent proteins in a cell; to activate a T cell; to modulate cells such that the cells are resistant to exhaustion or dysfunction; and/or increase the differentiation and/or proliferation of functionally exhausted or dysfunctional CD8+ T-cells (see PCT Patent Publications: WO2013176915, WO2014059173, WO2014172606, WO2014184744, and WO2014191128).

In certain embodiments, editing or other modification may result in inactivation of a gene. By inactivating a gene, it is intended that the gene of interest is not expressed in a functional protein form. In a particular embodiment, a CRISPR system specifically catalyzes cleavage in one targeted gene thereby inactivating said targeted gene. The nucleic acid strand breaks caused are commonly repaired through the distinct mechanisms of homologous recombination or non-homologous end joining (NHEJ). However, NHEJ is an imperfect repair process that often results in changes to the DNA sequence at the site of the cleavage. Repair via non-homologous end joining (NHEJ) often results in small insertions or deletions (Indel) and can be used for the creation of specific gene knockouts. Cells in which a cleavage induced mutagenesis event has occurred can be identified and/or selected by well-known methods in the art. In certain embodiments, homology directed repair (HDR) is used to concurrently inactivate a gene (e.g., TRAC) and insert an endogenous TCR or CAR into the inactivated locus.

Hence, in certain embodiments, editing of cells (such as by CRISPR/Cas) or modification by other systems, particularly cells intended for adoptive cell therapies, more particularly immunoresponsive cells such as T cells, may be performed to insert or knock-in an exogenous gene, such as an exogenous gene encoding a CAR or a TCR, at a preselected locus in a cell. Conventionally, nucleic acid molecules encoding CARs or TCRs are transfected or transduced to cells using randomly integrating vectors, which, depending on the site of integration, may lead to clonal expansion, oncogenic transformation, variegated transgene expression and/or transcriptional silencing of the transgene. Directing of transgene(s) to a specific locus in a cell can minimize or avoid such risks and advantageously provide for uniform expression of the transgene(s) by the cells. Without limitation, suitable ‘safe harbor’ loci for directed transgene integration include CCR5 or AAVS1. Homology-directed repair (HDR) strategies are known and described elsewhere in this specification allowing to insert transgenes into desired loci (e.g., TRAC locus).

Further suitable loci for insertion of transgenes, in particular CAR or exogenous TCR transgenes, include without limitation loci comprising genes coding for constituents of endogenous T-cell receptor, such as T-cell receptor alpha locus (TRA) or T-cell receptor beta locus (TRB), for example T-cell receptor alpha constant (TRAC) locus, T-cell receptor beta constant 1 (TRBC1) locus or T-cell receptor beta constant 2 (TRBC1) locus. Advantageously, insertion of a transgene into such locus can simultaneously achieve expression of the transgene, potentially controlled by the endogenous promoter, and knock-out expression of the endogenous TCR. This approach has been exemplified in Eyquem et al., (2017) Nature 543: 113-117, wherein the authors used CRISPR/Cas9 gene editing to knock-in a DNA molecule encoding a CD19-specific CAR into the TRAC locus downstream of the endogenous promoter; the CAR-T cells obtained by CRISPR were significantly superior in terms of reduced tonic CAR signaling and exhaustion.

T cell receptors (TCR) are cell surface receptors that participate in the activation of T cells in response to the presentation of antigen. The TCR is generally made from two chains, a and, which assemble to form a heterodimer and associates with the CD3-transducing subunits to form the T cell receptor complex present on the cell surface. Each a and p chain of the TCR consists of an immunoglobulin-like N-terminal variable (V) and constant (C) region, a hydrophobic transmembrane domain, and a short cytoplasmic region. As for immunoglobulin molecules, the variable region of the α and β chains are generated by V(D)J recombination, creating a large diversity of antigen specificities within the population of T cells. However, in contrast to immunoglobulins that recognize intact antigen, T cells are activated by processed peptide fragments in association with an MHC molecule, introducing an extra dimension to antigen recognition by T cells, known as MHC restriction. Recognition of MHC disparities between the donor and recipient through the T cell receptor leads to T cell proliferation and the potential development of graft versus host disease (GVHD). The inactivation of TCRα or TCRβ can result in the elimination of the TCR from the surface of T cells preventing recognition of alloantigen and thus GVHD. However, TCR disruption generally results in the elimination of the CD3 signaling component and alters the means of further T cell expansion.

Hence, in certain embodiments, editing or other modification of cells (such as by CRISPR/Cas), particularly cells intended for adoptive cell therapies, more particularly immunoresponsive cells such as T cells, may be performed to knock-out or knock-down expression of an endogenous TCR in a cell. For example, NHEJ-based or HDR-based gene editing approaches can be employed to disrupt the endogenous TCR alpha and/or beta chain genes. For example, gene editing system or systems, such as CRISPR/Cas system or systems, can be designed to target a sequence found within the TCR beta chain conserved between the beta 1 and beta 2 constant region genes (TRBC1 and TRBC2) and/or to target the constant region of the TCR alpha chain (TRAC) gene.

Allogeneic cells are rapidly rejected by the host immune system. It has been demonstrated that allogeneic leukocytes present in non-irradiated blood products will persist for no more than 5 to 6 days (Boni, Muranski et al. 2008 Blood 1; 112(12):4746-54). Thus, to prevent rejection of allogeneic cells, the host's immune system usually has to be suppressed to some extent. However, in the case of adoptive cell transfer the use of immunosuppressive drugs also have a detrimental effect on the introduced therapeutic T cells. Therefore, to effectively use an adoptive immunotherapy approach in these conditions, the introduced cells would need to be resistant to the immunosuppressive treatment. Thus, in a particular embodiment, the present invention further comprises a step of modifying T cells to make them resistant to an immunosuppressive agent, preferably by inactivating at least one gene encoding a target for an immunosuppressive agent. An immunosuppressive agent is an agent that suppresses immune function by one of several mechanisms of action. An immunosuppressive agent can be, but is not limited to a calcineurin inhibitor, a target of rapamycin, an interleukin-2 receptor α-chain blocker, an inhibitor of inosine monophosphate dehydrogenase, an inhibitor of dihydrofolic acid reductase, a corticosteroid or an immunosuppressive antimetabolite. The present invention allows conferring immunosuppressive resistance to T cells for immunotherapy by inactivating the target of the immunosuppressive agent in T cells. As non-limiting examples, targets for an immunosuppressive agent can be a receptor for an immunosuppressive agent such as: CD52, glucocorticoid receptor (GR), a FKBP family gene member and a cyclophilin family gene member.

In certain embodiments, editing or modification of cells (such as by CRISPR/Cas), particularly cells intended for adoptive cell therapies, more particularly immunoresponsive cells such as T cells, may be performed to block an immune checkpoint, such as to knock-out or knock-down expression of an immune checkpoint protein or receptor in a cell. Immune checkpoints are inhibitory pathways that slow down or stop immune reactions and prevent excessive tissue damage from uncontrolled activity of immune cells. In certain embodiments, the immune checkpoint targeted is the programmed death-1 (PD-1 or CD279) gene (PDCD1) (see, e.g., Rupp U, Schumann K, Roybal K T, et al. CRISPR/Cas9-mediated PD-1 disruption enhances anti-tumor efficacy of human chimeric antigen receptor T cells. Sci Rep. 2017; 7(1):737). In other embodiments, the immune checkpoint targeted is cytotoxic T-lymphocyte-associated antigen (CTLA-4). In additional embodiments, the immune checkpoint targeted is another member of the CD28 and CTLA4 Ig superfamily such as BTLA, LAG3, ICOS, PDL 1 or KR. In further additional embodiments, the immune checkpoint targeted is a member of the TNFR superfamily such as CD40, OX40, CD137, GITR, CD27 or TIM-3.

Additional immune checkpoints include Src homology 2 domain-containing protein tyrosine phosphatase 1 (SHP-1) (Watson H A, et al., SHP-1: the next checkpoint target for cancer immunotherapy? Biochem Soc Trans. 2016 Apr. 15; 44(2):356-62). SHP-1 is a widely expressed inhibitory protein tyrosine phosphatase (PTP). In T-cells, it is a negative regulator of antigen-dependent activation and proliferation. It is a cytosolic protein, and therefore not amenable to antibody-mediated therapies, but its role in activation and proliferation makes it an attractive target for genetic manipulation in adoptive transfer strategies, such as chimeric antigen receptor (CAR) T cells. Immune checkpoints may also include T cell immunoreceptor with Ig and ITIM domains (TIGIT/Vstm3/WUCAM/VSIG9) and VISTA (Le Mercier I, et al., (2015) Beyond CTLA-4 and PD-1, the generation Z of negative checkpoint regulators. Front. Immunol. 6:418).

WO2014172606 relates to the use of MT1 and/or MT2 inhibitors to increase proliferation and/or activity of exhausted CD8+ T-cells and to decrease CD8+ T-cell exhaustion (e.g., decrease functionally exhausted or unresponsive CD8+ immune cells). In certain embodiments, metallothioneins are targeted by gene editing in adoptively transferred T cells.

In certain embodiments, targets of gene editing may be at least one targeted locus involved in the expression of an immune checkpoint protein. Such targets may include, but are not limited to CTLA4, PPP2CA, PPP2CB, PTPN6, PTPN22, PDCD1, ICOS (CD278), PDL1, KIR, LAG3, HAVCR2, BTLA, CD160, TIGIT, CD96, CRTAM, LAIR1, SIGLEC7, SIGLEC9, CD244 (2B4), TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, VISTA, GUCYlA2, GUCYlA3, GUCYlB2, GUCY1B3, MT1, MT2, CD40, OX40, CD137, GITR, CD27, SHP-1, TIM-3, CEACAM-1, CEACAM-3, or CEACAM-5. In preferred embodiments, the gene locus involved in the expression of PD-1 or CTLA-4 genes is targeted. In other preferred embodiments, combinations of genes are targeted, such as, but not limited to, PD-1 and TIGIT.

By means of an example and without limitation, WO2016196388 concerns an engineered T cell comprising (a) a genetically engineered antigen receptor that specifically binds to an antigen, which receptor may be a CAR; and (b) a disrupted gene encoding a PD-L1, an agent for disruption of a gene encoding a PD-L1, and/or disruption of a gene encoding PD-L1, wherein the disruption of the gene may be mediated by a gene editing nuclease, a zinc finger nuclease (ZFN), CRISPR/Cas9 and/or TALEN. WO2015142675 relates to immune effector cells comprising a CAR in combination with an agent (such as CRISPR, TALEN or ZFN) that increases the efficacy of the immune effector cells in the treatment of cancer, wherein the agent may inhibit an immune inhibitory molecule, such as PD1, PD-L1, CTLA-4, TIM-3, LAG-3, VISTA, BTLA, TIGIT, LAIR1, CD160, 2B4, TGFR beta, CEACAM-1, CEACAM-3, or CEACAM-5. Ren et al., (2017) Clin Cancer Res 23 (9) 2255-2266 performed lentiviral delivery of CAR and electro-transfer of Cas9 mRNA and gRNAs targeting endogenous TCR, J-2 microglobulin (B2M) and PD1 simultaneously, to generate gene-disrupted allogeneic CAR T cells deficient of TCR, HLA class I molecule and PD1.

In certain embodiments, cells may be engineered to express a CAR, wherein expression and/or function of methylcytosine dioxygenase genes (TET1, TET2 and/or TET3) in the cells has been reduced or eliminated, such as by CRISPR, ZNF or TALEN (for example, as described in WO201704916).

In certain embodiments, editing of cells (such as by CRISPR/Cas), particularly cells intended for adoptive cell therapies, more particularly immunoresponsive cells such as T cells, may be performed to knock-out or knock-down expression of an endogenous gene in a cell, said endogenous gene encoding an antigen targeted by an exogenous CAR or TCR, thereby reducing the likelihood of targeting of the engineered cells. In certain embodiments, the targeted antigen may be one or more antigen selected from the group consisting of CD38, CD138, CS-1, CD33, CD26, CD30, CD53, CD92, CD100, CD148, CD150, CD200, CD261, CD262, CD362, human telomerase reverse transcriptase (hTERT), survivin, mouse double minute 2 homolog (MDM2), cytochrome P450 1B1 (CYP1B), HER2/neu, Wilms' tumor gene 1 (WT1), livin, alphafetoprotein (AFP), carcinoembryonic antigen (CEA), mucin 16 (MUC16), MUC1, prostate-specific membrane antigen (PSMA), p53, cyclin (D1), B cell maturation antigen (BCMA), transmembrane activator and CAML Interactor (TACI), and B-cell activating factor receptor (BAFF-R) (for example, as described in WO2016011210 and WO2017011804).

In certain embodiments, editing or modification of cells (such as by CRISPR/Cas), particularly cells intended for adoptive cell therapies, more particularly immunoresponsive cells such as T cells, may be performed to knock-out or knock-down expression of one or more MHC constituent proteins, such as one or more HLA proteins and/or beta-2 microglobulin (B2M), in a cell, whereby rejection of non-autologous (e.g., allogeneic) cells by the recipient's immune system can be reduced or avoided. In preferred embodiments, one or more HLA class I proteins, such as HLA-A, B and/or C, and/or B2M may be knocked-out or knocked-down. Preferably, B2M may be knocked-out or knocked-down. By means of an example, Ren et al., (2017) Clin Cancer Res 23 (9) 2255-2266 performed lentiviral delivery of CAR and electro-transfer of Cas9 mRNA and gRNAs targeting endogenous TCR, J-2 microglobulin (B2M) and PD1 simultaneously, to generate gene-disrupted allogeneic CAR T cells deficient of TCR, HLA class I molecule and PD1.

In other embodiments, at least two genes are edited. Pairs of genes may include, but are not limited to PD1 and TCRα, PD1 and TCRβ, CTLA-4 and TCRα, CTLA-4 and TCRβ, LAG3 and TCRα, LAG3 and TCRβ, Tim3 and TCRα, Tim3 and TCRβ, BTLA and TCRα, BTLA and TCRβ, BY55 and TCRα, BY55 and TCRβ, TIGIT and TCRα, TIGIT and TCRβ, B7H5 and TCRα, B7H5 and TCRβ, LAIR1 and TCRα, LAIR1 and TCRβ, SIGLEC10 and TCRα, SIGLEC10 and TCRβ, 2B4 and TCRα, 2B4 and TCRβ, B2M and TCRα, B2M and TCRβ.

In certain embodiments, a cell may be multiply edited (multiplex genome editing) as taught herein to (1) knock-out or knock-down expression of an endogenous TCR (for example, TRBC1, TRBC2 and/or TRAC), (2) knock-out or knock-down expression of an immune checkpoint protein or receptor (for example PD1, PD-L1 and/or CTLA4); and (3) knock-out or knock-down expression of one or more MHC constituent proteins (for example, HLA-A, B and/or C, and/or B2M, preferably B2M).

Whether prior to or after genetic modification of the T cells, the T cells can be activated and expanded generally using methods as described, for example, in U.S. Pat. Nos. 6,352,694; 6,534,055; 6,905,680; 5,858,358; 6,887,466; 6,905,681; 7,144,575; 7,232,566; 7,175,843; 5,883,223; 6,905,874; 6,797,514; 6,867,041; and 7,572,631. T cells can be expanded in vitro or in vivo.

Immune cells may be obtained using any method known in the art. In one embodiment, allogenic T cells may be obtained from healthy subjects. In one embodiment T cells that have infiltrated a tumor are isolated. T cells may be removed during surgery. T cells may be isolated after removal of tumor tissue by biopsy. T cells may be isolated by any means known in the art. In one embodiment, T cells are obtained by apheresis. In one embodiment, the method may comprise obtaining a bulk population of T cells from a tumor sample by any suitable method known in the art. For example, a bulk population of T cells can be obtained from a tumor sample by dissociating the tumor sample into a cell suspension from which specific cell populations can be selected. Suitable methods of obtaining a bulk population of T cells may include, but are not limited to, any one or more of mechanically dissociating (e.g., mincing) the tumor, enzymatically dissociating (e.g., digesting) the tumor, and aspiration (e.g., as with a needle).

The bulk population of T cells obtained from a tumor sample may comprise any suitable type of T cell. Preferably, the bulk population of T cells obtained from a tumor sample comprises tumor infiltrating lymphocytes (TILs).

The tumor sample may be obtained from any mammal. Unless stated otherwise, as used herein, the term “mammal” refers to any mammal including, but not limited to, mammals of the order Logomorpha, such as rabbits; the order Carnivora, including Felines (cats) and Canines (dogs); the order Artiodactyla, including Bovines (cows) and Swines (pigs); or of the order Perssodactyla, including Equines (horses). The mammals may be non-human primates, e.g., of the order Primates, Ceboids, or Simoids (monkeys) or of the order Anthropoids (humans and apes). In some embodiments, the mammal may be a mammal of the order Rodentia, such as mice and hamsters. Preferably, the mammal is a non-human primate or a human. An especially preferred mammal is the human.

T cells can be obtained from a number of sources, including peripheral blood mononuclear cells (PBMC), bone marrow, lymph node tissue, spleen tissue, and tumors. In certain embodiments of the present invention, T cells can be obtained from a unit of blood collected from a subject using any number of techniques known to the skilled artisan, such as Ficoll separation. In one preferred embodiment, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In one embodiment of the invention, the cells are washed with phosphate buffered saline (PBS). In an alternative embodiment, the wash solution lacks calcium and may lack magnesium or may lack many if not all divalent cations. Initial activation steps in the absence of calcium lead to magnified activation. As those of ordinary skill in the art would readily appreciate, a washing step may be accomplished by methods known to those in the art, such as by using a semi-automated “flow-through” centrifuge (for example, the Cobe 2991 cell processor) according to the manufacturer's instructions. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In another embodiment, T cells are isolated from peripheral blood lymphocytes by lysing the red blood cells and depleting the monocytes, for example, by centrifugation through a PERCOLL™ gradient. A specific subpopulation of T cells, such as CD28+, CD4+, CDC, CD45RA+, and CD45RO+ T cells, can be further isolated by positive or negative selection techniques. For example, in one preferred embodiment, T cells are isolated by incubation with anti-CD3/anti-CD28 (i.e., 3×28)-conjugated beads, such as DYNABEADS® M-450 CD3/CD28 T, or XCYTE DYNABEADS™ for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another preferred embodiment, the time period is 10 to 24 hours. In one preferred embodiment, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells.

Enrichment of a T cell population by negative selection can be accomplished with a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8.

Further, monocyte populations (i.e., CD14+ cells) may be depleted from blood preparations by a variety of methodologies, including anti-CD14 coated beads or columns, or utilization of the phagocytotic activity of these cells to facilitate removal. Accordingly, in one embodiment, the invention uses paramagnetic particles of a size sufficient to be engulfed by phagocytotic monocytes. In certain embodiments, the paramagnetic particles are commercially available beads, for example, those produced by Life Technologies under the trade name Dynabeads™. In one embodiment, other non-specific cells are removed by coating the paramagnetic particles with “irrelevant” proteins (e.g., serum proteins or antibodies). Irrelevant proteins and antibodies include those proteins and antibodies or fragments thereof that do not specifically target the T cells to be isolated. In certain embodiments, the irrelevant beads include beads coated with sheep anti-mouse antibodies, goat anti-mouse antibodies, and human serum albumin.

In brief, such depletion of monocytes is performed by preincubating T cells isolated from whole blood, apheresed peripheral blood, or tumors with one or more varieties of irrelevant or non-antibody coupled paramagnetic particles at any amount that allows for removal of monocytes (approximately a 20:1 bead:cell ratio) for about 30 minutes to 2 hours at 22 to 37 degrees C., followed by magnetic removal of cells which have attached to or engulfed the paramagnetic particles. Such separation can be performed using standard methods available in the art. For example, any magnetic separation methodology may be used including a variety of which are commercially available, (e.g., DYNAL® Magnetic Particle Concentrator (DYNAL MPC®)). Assurance of requisite depletion can be monitored by a variety of methodologies known to those of ordinary skill in the art, including flow cytometric analysis of CD14 positive cells, before and after depletion.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc.). Such populations of cells may have therapeutic value and would be desirable to obtain. For example, using high concentration of cells allows more efficient selection of CD8+ T cells that normally have weaker CD28 expression.

In a related embodiment, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In one embodiment, the concentration of cells used is 5×10⁶/ml. In other embodiments, the concentration used can be from about 1×10⁵/ml to 1×10⁶/ml, and any integer value in between.

T cells can also be frozen. Wishing not to be bound by theory, the freeze and subsequent thaw step provides a more uniform product by removing granulocytes and to some extent monocytes in the cell population. After a washing step to remove plasma and platelets, the cells may be suspended in a freezing solution. While many freezing solutions and parameters are known in the art and will be useful in this context, one method involves using PBS containing 20% DMSO and 8% human serum albumin, or other suitable cell freezing media, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

T cells for use in the present invention may also be antigen-specific T cells. For example, tumor-specific T cells can be used. In certain embodiments, antigen-specific T cells can be isolated from a patient of interest, such as a patient afflicted with a cancer or an infectious disease. In one embodiment, neoepitopes are determined for a subject and T cells specific to these antigens are isolated. Antigen-specific cells for use in expansion may also be generated in vitro using any number of methods known in the art, for example, as described in U.S. Patent Publication No. US 20040224402 entitled, Generation and Isolation of Antigen-Specific T Cells, or in U.S. Pat. Nos. 6,040,177. Antigen-specific cells for use in the present invention may also be generated using any number of methods known in the art, for example, as described in Current Protocols in Immunology, or Current Protocols in Cell Biology, both published by John Wiley & Sons, Inc., Boston, Mass.

In a related embodiment, it may be desirable to sort or otherwise positively select (e.g., via magnetic selection) the antigen specific cells prior to or following one or two rounds of expansion. Sorting or positively selecting antigen-specific cells can be carried out using peptide-MHC tetramers (Altman, et al., Science. 1996 Oct. 4; 274(5284):94-6). In another embodiment, the adaptable tetramer technology approach is used (Andersen et al., 2012 Nat Protoc. 7:891-902). Tetramers are limited by the need to utilize predicted binding peptides based on prior hypotheses, and the restriction to specific HLAs. Peptide-MHC tetramers can be generated using techniques known in the art and can be made with any MHC molecule of interest and any antigen of interest as described herein. Specific epitopes to be used in this context can be identified using numerous assays known in the art. For example, the ability of a polypeptide to bind to MHC class I may be evaluated indirectly by monitoring the ability to promote incorporation of ¹²⁵I labeled β2-microglobulin (β2 m) into MHC class I/β2m/peptide heterotrimeric complexes (see Parker et al., J. Immunol. 152:163, 1994).

In one embodiment, cells are directly labeled with an epitope-specific reagent for isolation by flow cytometry followed by characterization of phenotype and TCRs. In one embodiment, T cells are isolated by contacting with T cell specific antibodies. Sorting of antigen-specific T cells, or generally any cells of the present invention, can be carried out using any of a variety of commercially available cell sorters, including, but not limited to, MoFlo sorter (DakoCytomation, Fort Collins, Colo.), FACSAria™ sorter, FACSArray™ sorter, FACSVantage™ sorter, BD™ LSR II sorter, and FACSCalibur™ sorter (BD Biosciences, San Jose, Calif.).

In a preferred embodiment, the method comprises selecting cells that also express CD3. The method may comprise specifically selecting the cells in any suitable manner. Preferably, the selecting is carried out using flow cytometry. The flow cytometry may be carried out using any suitable method known in the art. The flow cytometry may employ any suitable antibodies and stains. Preferably, the antibody is chosen such that it specifically recognizes and binds to the particular biomarker being selected. For example, the specific selection of CD3, CD8, TIM-3, LAG-3, 4-1BB, or PD-1 may be carried out using anti-CD3, anti-CD8, anti-TIM-3, anti-LAG-3, anti-4-1BB, or anti-PD-1 antibodies, respectively. The antibody or antibodies may be conjugated to a bead (e.g., a magnetic bead) or to a fluorochrome. Preferably, the flow cytometry is fluorescence-activated cell sorting (FACS). TCRs expressed on T cells can be selected based on reactivity to autologous tumors. Additionally, T cells that are reactive to tumors can be selected based on markers using the methods described in patent publication Nos. WO2014133567 and WO2014133568, herein incorporated by reference in their entirety. Additionally, activated T cells can be selected based on surface expression of CD107a.

In one embodiment of the invention, the method further comprises expanding the numbers of T cells in the enriched cell population. Such methods are described in U.S. Pat. No. 8,637,307 and is herein incorporated by reference in its entirety. The numbers of T cells may be increased at least about 3-fold (or 4-, 5-, 6-, 7-, 8-, or 9-fold), more preferably at least about 10-fold (or 20-, 30-, 40-, 50-, 60-, 70-, 80-, or 90-fold), more preferably at least about 100-fold, more preferably at least about 1,000 fold, or most preferably at least about 100,000-fold. The numbers of T cells may be expanded using any suitable method known in the art. Exemplary methods of expanding the numbers of cells are described in patent publication No. WO 2003057171, U.S. Pat. No. 8,034,334, and U.S. Patent Application Publication No. 2012/0244133, each of which is incorporated herein by reference.

In one embodiment, ex vivo T cell expansion can be performed by isolation of T cells and subsequent stimulation or activation followed by further expansion. In one embodiment of the invention, the T cells may be stimulated or activated by a single agent. In another embodiment, T cells are stimulated or activated with two agents, one that induces a primary signal and a second that is a co-stimulatory signal. Ligands useful for stimulating a single signal or stimulating a primary signal and an accessory molecule that stimulates a second signal may be used in soluble form. Ligands may be attached to the surface of a cell, to an Engineered Multivalent Signaling Platform (EMSP), or immobilized on a surface. In a preferred embodiment, both primary and secondary agents are co-immobilized on a surface, for example a bead or a cell. In one embodiment, the molecule providing the primary activation signal may be a CD3 ligand, and the co-stimulatory molecule may be a CD28 ligand or 4-1BB ligand.

In certain embodiments, T cells comprising a CAR or an exogenous TCR, may be manufactured as described in WO2015120096, by a method comprising: enriching a population of lymphocytes obtained from a donor subject; stimulating the population of lymphocytes with one or more T-cell stimulating agents to produce a population of activated T cells, wherein the stimulation is performed in a closed system using serum-free culture medium; transducing the population of activated T cells with a viral vector comprising a nucleic acid molecule which encodes the CAR or TCR, using a single cycle transduction to produce a population of transduced T cells, wherein the transduction is performed in a closed system using serum-free culture medium; and expanding the population of transduced T cells for a predetermined time to produce a population of engineered T cells, wherein the expansion is performed in a closed system using serum-free culture medium. In certain embodiments, T cells comprising a CAR or an exogenous TCR, may be manufactured as described in WO2015120096, by a method comprising: obtaining a population of lymphocytes; stimulating the population of lymphocytes with one or more stimulating agents to produce a population of activated T cells, wherein the stimulation is performed in a closed system using serum-free culture medium; transducing the population of activated T cells with a viral vector comprising a nucleic acid molecule which encodes the CAR or TCR, using at least one cycle transduction to produce a population of transduced T cells, wherein the transduction is performed in a closed system using serum-free culture medium; and expanding the population of transduced T cells to produce a population of engineered T cells, wherein the expansion is performed in a closed system using serum-free culture medium. The predetermined time for expanding the population of transduced T cells may be 3 days. The time from enriching the population of lymphocytes to producing the engineered T cells may be 6 days. The closed system may be a closed bag system. Further provided is population of T cells comprising a CAR or an exogenous TCR obtainable or obtained by said method, and a pharmaceutical composition comprising such cells.

In certain embodiments, T cell maturation or differentiation in vitro may be delayed or inhibited by the method as described in WO2017070395, comprising contacting one or more T cells from a subject in need of a T cell therapy with an AKT inhibitor (such as, e.g., one or a combination of two or more AKT inhibitors disclosed in claim 8 of WO2017070395) and at least one of exogenous Interleukin-7 (IL-7) and exogenous Interleukin-15 (IL-15), wherein the resulting T cells exhibit delayed maturation or differentiation, and/or wherein the resulting T cells exhibit improved T cell function (such as, e.g., increased T cell proliferation; increased cytokine production; and/or increased cytolytic activity) relative to a T cell function of a T cell cultured in the absence of an AKT inhibitor.

In certain embodiments, a patient in need of a T cell therapy may be conditioned by a method as described in WO2016191756 comprising administering to the patient a dose of cyclophosphamide between 200 mg/m2/day and 2000 mg/m2/day and a dose of fludarabine between 20 mg/m2/day and 900 mg/m²/day.

In certain embodiments, a patient in need of adoptive cell transfer may be administered a TLR agonist to enhance anti-tumor immunity (see, e.g., Urban-Wojciuk, et al., The Role of TLRs in Anti-cancer Immunity and Tumor Rejection, Front Immunol. 2019; 10: 2388; and Kaczanowska et al., TLR agonists: our best frenemy in cancer immunotherapy, J Leukoc Biol. 2013 June; 93(6): 847-863). In certain embodiments, TLR agonists are delivered in a nanoparticle system (see, e.g., Buss and Bhatia, Nanoparticle delivery of immunostimulatory oligonucleotides enhances response to checkpoint inhibitor therapeutics, Proc Natl Acad Sci USA. 2020 Jun. 3; 202001569). In certain embodiments, the agonist is a TLR9 agonist. Id.

### Bioreactors

In some embodiments the cell is a bioreactor that can produce one or more bioproducts. In some embodiments, the bioreactor cell is a mammalian cell, an insect cell, a bacterial cell, a fungal cell, a plant cell, or an algae. Exemplary bioreactor systems for the production of bioproducts are generally known in the art. As used herein, “bioproduct” refers to any biological molecule that can be produced in or by a cell. Such molecules include, without limitation, amino acids, peptides, polypeptides, nucleic acids, polynucleotides, oligonucleotides, lipids, small chemical molecules (e.g., antibiotics and other medications), etc. In some embodiments, productivity or a characteristic of a bioreactor can be improved by decreasing one or more target proteins in the bioreactor cell. For example, depletion of protein(s) that decrease the yield, decrease the efficiency of production, or those that otherwise interfere with or limit the production of the bioproduct would be advantageous.

### Pharmaceutical Formulations

Also described herein are pharmaceutical formulations that can contain an amount, effective amount, and/or least effective amount, and/or therapeutically effective amount of one or more compounds, molecules, compositions, vectors, vector systems, cells, or a combination thereof (which are also referred to as the primary active agent or ingredient elsewhere herein) described in greater detail elsewhere herein and a pharmaceutically acceptable carrier or excipient. As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo. As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient. When present, the compound can optionally be present in the pharmaceutical formulation as a pharmaceutically acceptable salt. In some embodiments, the pharmaceutical formulation can include, such as an active ingredient, a CRISPR-Cas system or component thereof described in greater detail elsewhere herein. In some embodiments, the pharmaceutical formulation can include, such as an active ingredient, a CRISPR-Cas polynucleotide described in greater detail elsewhere herein. In some embodiments, the pharmaceutical formulation can include an active ingredient, such as one or more modified cells, such as one or more modified cells described in greater detail elsewhere herein.

In some embodiments, the active ingredient is present as a pharmaceutically acceptable salt of the active ingredient. As used herein, “pharmaceutically acceptable salt” refers to any acid or base addition salt whose counter-ions are non-toxic to the subject to which they are administered in pharmaceutical doses of the salts. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

The pharmaceutical formulations described herein can be administered to a subject in need thereof via any suitable method or route to a subject in need thereof. Suitable administration routes can include, but are not limited to auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated and/or the active ingredient(s).

Where appropriate, compounds, molecules, compositions, vectors, vector systems, cells, or a combination thereof described in greater detail elsewhere herein can be provided to a subject in need thereof as an ingredient, such as an active ingredient or agent, in a pharmaceutical formulation. As such, also described are pharmaceutical formulations containing one or more of the compounds and salts thereof, or pharmaceutically acceptable salts thereof described herein. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

In some embodiments, the subject in need thereof has or is suspected of having a cancer or a symptom thereof. In some embodiments, the subject in need thereof has or is suspected of having, a neurobiological disease or disorder, a psychiatric disease or disorder, an autoimmune disease or disorder, a thrombosis disease, a heart disease, a kidney disease, a lung disease, hematopoietic disease. Or a blood vessel disease, or a combination thereof. As used herein, “agent” refers to any substance, compound, molecule, and the like, which can be biologically active or otherwise can induce a biological and/or physiological effect on a subject to which it is administered to. As used herein, “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

### Pharmaceutically Acceptable Carriers and Secondary Ingredients and Agents

The pharmaceutical formulation can include a pharmaceutically acceptable carrier. Suitable pharmaceutically acceptable carriers include, but are not limited to water, salt solutions, alcohols, gum rabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with agents, such as lubricants, preservatives, stabilizers, wetting agents, emulsifiers, salts for influencing osmotic pressure, buffers, coloring, flavoring and/or aromatic substances, and the like which do not deleteriously react with the active compound.

In some embodiments, the pharmaceutical formulation can also include an effective amount of secondary active agents, including but not limited to, biologic agents or molecules including, but not limited to, e.g., polynucleotides, amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives, chemotherapeutics, and combinations thereof.

### Effective Amounts

In some embodiments, the amount of the primary active agent and/or optional secondary agent can be an effective amount, least effective amount, and/or therapeutically effective amount. As used herein, “effective amount” refers to the amount of the primary and/or optional secondary agent included in the pharmaceutical formulation that achieves one or more therapeutic effects or desired effect. As used herein, “least effective” amount refers to the lowest amount of the primary and/or optional secondary agent that achieves the one or more therapeutic or other desired effects. As used herein, “therapeutically effective amount” refers to the amount of the primary and/or optional secondary agent included in the pharmaceutical formulation that achieves one or more therapeutic effects. In some embodiments, the one or more therapeutic effects are to reduce the amount of a target protein in a cell or cells. In some embodiments, the one or more therapeutic effects of a pharmaceutical formulation containing an engineered bifunctional receptor are the reduction of cellular dysfunction, exhaustion, and/or transformation, particularly in a cell used and/or engineered for use as a cell therapy, including but not limited to an adoptive cell therapy.

The effective amount, least effective amount, and/or therapeutically effective amount of the primary and optional secondary active agent described elsewhere herein contained in the pharmaceutical formulation can be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, g, mg, or g or be any numerical value or subrange within any of these ranges.

In some embodiments, the effective amount, least effective amount, and/or therapeutically effective amount can be an effective concentration, least effective concentration, and/or therapeutically effective concentration, which can each be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 μM, nM, M, mM, or M or be any numerical value or subrange within any of these ranges.

In other embodiments, the effective amount, least effective amount, and/or therapeutically effective amount of the primary and optional secondary active agent be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 IU or be any numerical value or subrange within any of these ranges.

In some embodiments, the primary and/or the optional secondary active agent present in the pharmaceutical formulation can be any non-zero amount ranging from about 0 to 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.9, to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the pharmaceutical formulation or be any numerical value or subrange within any of these ranges.

In some embodiments where a cell or cell population is present in the pharmaceutical formulation (e.g., as a primary and/or or secondary active agent), the effective amount of cells can be any amount ranging from about 1 or 2 cells to 1×10¹/mL, 1×10²⁰/mL or more, such as about 1×10¹/mL, 1×10²/mL, 1×10³/mL, 1×10⁴/mL, 1×10⁵/mL, 1×10⁶/mL, 1×10⁷/mL, 1×10⁸/mL, 1×10⁹/mL, 1×10¹⁰/mL, 1×10¹¹/mL, 1×10¹²/mL, 1×10¹³/mL, 1×10¹⁴/mL, 1×10¹⁵/mL, 1×10¹⁶/mL, 1×10¹⁷/mL, 1×10¹⁸/mL, 1×10¹⁹/mL, to/or about 1×10²⁰/mL or any numerical value or subrange within any of these ranges.

In some embodiments, the amount or effective amount, particularly where an infective particle is being delivered (e.g., a virus particle having the primary or secondary agent as a cargo), the effective amount of virus particles can be expressed as a titer (plaque forming units per unit of volume) or as a MOI (multiplicity of infection). In some embodiments, the effective amount can be about 1×10¹ particles per pL, nL, μL, mL, or L to 1×10²⁰/particles per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ particles per pL, nL, μL, mL, or L. In some embodiments, the effective titer can be about 1×10¹ transforming units per pL, nL, μL, mL, or L to 1×10²⁰/transforming units per pL, nL, L, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ transforming units per pL, nL, μL, mL, or L or any numerical value or subrange within these ranges. In some embodiments, the MOI of the pharmaceutical formulation can range from about 0.1 to 10 or more, such as 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10 or more or any numerical value or subrange within these ranges.

In some embodiments, the amount or effective amount of the one or more of the active agent(s) described herein contained in the pharmaceutical formulation can range from about 1 μg/kg to about 10 mg/kg based upon the bodyweight of the subject in need thereof or average bodyweight of the specific patient population to which the pharmaceutical formulation can be administered.

In embodiments where there is a secondary agent contained in the pharmaceutical formulation, the effective amount of the secondary active agent will vary depending on the secondary agent, the primary agent, the administration route, subject age, disease, stage of disease, among other things, which will be one of ordinary skill in the art.

When optionally present in the pharmaceutical formulation, the secondary active agent can be included in the pharmaceutical formulation or can exist as a stand-alone compound or pharmaceutical formulation that can be administered contemporaneously or sequentially with the compound, derivative thereof, or pharmaceutical formulation thereof.

In some embodiments, the effective amount of the secondary active agent, when optionally present, is any non-zero amount ranging from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total active agents present in the pharmaceutical formulation or any numerical value or subrange within these ranges. In additional embodiments, the effective amount of the secondary active agent is any non-zero amount ranging from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total pharmaceutical formulation or any numerical value or subrange within these ranges.

### Dosage Forms

In some embodiments, the pharmaceutical formulations described herein can be provided in a dosage form. The dosage form can be administered to a subject in need thereof. The dosage form can be effective generate specific concentration, such as an effective concentration, at a given site in the subject in need thereof. As used herein, “dose,” “unit dose,” or “dosage” can refer to physically discrete units suitable for use in a subject, each unit containing a predetermined quantity of the primary active agent, and optionally present secondary active ingredient, and/or a pharmaceutical formulation thereof calculated to produce the desired response or responses in association with its administration. In some embodiments, the given site is proximal to the administration site. In some embodiments, the given site is distal to the administration site. In some cases, the dosage form contains a greater amount of one or more of the active ingredients present in the pharmaceutical formulation than the final intended amount needed to reach a specific region or location within the subject to account for loss of the active components such as via first and second pass metabolism.

The dosage forms can be adapted for administration by any appropriate route. Appropriate routes include, but are not limited to, oral (including buccal or sublingual), rectal, intraocular, inhaled, intranasal, topical (including buccal, sublingual, or transdermal), vaginal, parenteral, subcutaneous, intramuscular, intravenous, internasal, and intradermal. Other appropriate routes are described elsewhere herein. Such formulations can be prepared by any method known in the art.

Dosage forms adapted for oral administration can discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In some embodiments, the pharmaceutical formulations adapted for oral administration also include one or more agents which flavor, preserve, color, or help disperse the pharmaceutical formulation. Dosage forms prepared for oral administration can also be in the form of a liquid solution that can be delivered as a foam, spray, or liquid solution. The oral dosage form can be administered to a subject in need thereof. Where appropriate, the dosage forms described herein can be microencapsulated.

The dosage form can also be prepared to prolong or sustain the release of any ingredient. In some embodiments, compounds, molecules, compositions, vectors, vector systems, cells, or a combination thereof described herein can be the ingredient whose release is delayed. In some embodiments the primary active agent is the ingredient whose release is delayed. In some embodiments, an optional secondary agent can be the ingredient whose release is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20^{th} ed., Lippincott Williams & Wilkins, Baltimore, MD, 2000, and “Pharmaceutical dosage forms and drug delivery systems”, 6^{th} Edition, Ansel et al., (Media, PA: Williams and Wilkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water-soluble polymer, water insoluble polymers, and/or pH dependent polymers, with or without water insoluble/water soluble non-polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Where appropriate, the dosage forms described herein can be a liposome. In these embodiments, primary active ingredient(s), and/or optional secondary active ingredient(s), and/or pharmaceutically acceptable salt thereof where appropriate are incorporated into a liposome. In embodiments where the dosage form is a liposome, the pharmaceutical formulation is thus a liposomal formulation. The liposomal formulation can be administered to a subject in need thereof.

Dosage forms adapted for topical administration can be formulated as ointments, creams, suspensions, lotions, powders, solutions, pastes, gels, sprays, aerosols, or oils. In some embodiments for treatments of the eye or other external tissues, for example the mouth or the skin, the pharmaceutical formulations are applied as a topical ointment or cream. When formulated in an ointment, a primary active ingredient, optional secondary active ingredient, and/or pharmaceutically acceptable salt thereof where appropriate can be formulated with a paraffinic or water-miscible ointment base. In other embodiments, the primary and/or secondary active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In some embodiments, a primary active ingredient, optional secondary active ingredient, and/or pharmaceutically acceptable salt thereof where appropriate can be in a dosage form adapted for inhalation is in a particle-size-reduced form that is obtained or obtainable by micronization. In some embodiments, the particle size of the size reduced (e.g., micronized) compound or salt or solvate thereof, is defined by a D₅₀ value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active (primary and/or secondary) ingredient, which may be generated by various types of metered dose pressurized aerosols, nebulizers, or insufflators. The nasal/inhalation formulations can be administered to a subject in need thereof.

In some embodiments, the dosage forms are aerosol formulations suitable for administration by inhalation. In some of these embodiments, the aerosol formulation contains a solution or fine suspension of a primary active ingredient, secondary active ingredient, and/or pharmaceutically acceptable salt thereof where appropriate and a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal or an aerosol dispenser fitted with a metering valve (e.g., metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of a primary active ingredient, optional secondary active ingredient, and/or pharmaceutically acceptable salt thereof. In further embodiments, the aerosol formulation also contains co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, or 8 times daily, in which 1, 2, 3 or more doses are delivered each time. The aerosol formulations can be administered to a subject in need thereof.

For some dosage forms suitable and/or adapted for inhaled administration, the pharmaceutical formulation is a dry powder inhalable-formulations. In addition to a primary active agent, optional secondary active ingredient, and/or pharmaceutically acceptable salt thereof where appropriate, such a dosage form can contain a powder base such as lactose, glucose, trehalose, mannitol, and/or starch. In some of these embodiments, a primary active agent, secondary active ingredient, and/or pharmaceutically acceptable salt thereof where appropriate is in a particle-size reduced form. In further embodiments, a performance modifier, such as L-leucine or another amino acid, cellobiose octaacetate, and/or metals salts of stearic acid, such as magnesium or calcium stearate. In some embodiments, the aerosol formulations are arranged so that each metered dose of aerosol contains a predetermined amount of an active ingredient, such as the one or more of the compositions, compounds, vector(s), molecules, cells, and combinations thereof described herein.

Dosage forms adapted for vaginal administration can be presented as pessaries, tampons, creams, gels, pastes, foams, or spray formulations. Dosage forms adapted for rectal administration include suppositories or enemas. The vaginal formulations can be administered to a subject in need thereof.

Dosage forms adapted for parenteral administration and/or adapted for injection can include aqueous and/or non-aqueous sterile injection solutions, which can contain antioxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and re-suspended in a sterile carrier to reconstitute the dose prior to administration. Extemporaneous injection solutions and suspensions can be prepared in some embodiments, from sterile powders, granules, and tablets. The parenteral formulations can be administered to a subject in need thereof.

For some embodiments, the dosage form contains a predetermined amount of a primary active agent, secondary active ingredient, and/or pharmaceutically acceptable salt thereof where appropriate per unit dose. In an embodiment, the predetermined amount of primary active agent, secondary active ingredient, and/or pharmaceutically acceptable salt thereof where appropriate can be an effective amount, a least effect amount, and/or a therapeutically effective amount. In other embodiments, the predetermined amount of a primary active agent, secondary active agent, and/or pharmaceutically acceptable salt thereof where appropriate, can be an appropriate fraction of the effective amount of the active ingredient.

### Co-Therapies and Combination Therapies

In some embodiments, the pharmaceutical formulation(s) described herein are part of a combination treatment or combination therapy. The combination treatment can include the pharmaceutical formulation described herein and an additional treatment modality. The additional treatment modality can be a chemotherapeutic, a biological therapeutic, surgery, radiation, diet modulation, environmental modulation, a physical activity modulation, and combinations thereof.

In some embodiments, the co-therapy or combination therapy can additionally include but not limited to, polynucleotides, amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives, chemotherapeutics, and combinations thereof.

### Administration of the Pharmaceutical Formulations

The pharmaceutical formulations or dosage forms thereof described herein can be administered one or more times hourly, daily, monthly, or yearly (e.g., 1, 2, 3, 4, 5, 6, 7, 8, 9, 10, 11, 12, 13, 14, 15, 16, 17, 18, 19, 20, or more times hourly, daily, monthly, or yearly). In some embodiments, the pharmaceutical formulations or dosage forms thereof described herein can be administered continuously over a period of time ranging from minutes to hours to days. Device and dosage forms are known in the art and described herein that are effective to provide continuous administration of the pharmaceutical formulations described herein. In some embodiments, the first one or a few initial amount(s) administered can be a higher dose than subsequent doses. This is typically referred to in the art as a loading dose or doses and a maintenance dose, respectively. In some embodiments, the pharmaceutical formulations can be administered such that the doses over time are tapered (increased or decreased) overtime so as to wean a subject gradually off of a pharmaceutical formulation or gradually introduce a subject to the pharmaceutical formulation.

As previously discussed, the pharmaceutical formulation can contain a predetermined amount of a primary active agent, secondary active agent, and/or pharmaceutically acceptable salt thereof where appropriate. In some of these embodiments, the predetermined amount can be an appropriate fraction of the effective amount of the active ingredient. Such unit doses may therefore be administered once or more than once a day, month, or year (e.g., 1, 2, 3, 4, 5, 6, or more times per day, month, or year). Such pharmaceutical formulations may be prepared by any of the methods well known in the art.

Where co-therapies or multiple pharmaceutical formulations are to be delivered to a subject, the different therapies or formulations can be administered sequentially or simultaneously. Sequential administration is administration where an appreciable amount of time occurs between administrations, such as more than about 15, 20, 30, 45, 60 minutes or more. The time between administrations in sequential administration can be on the order of hours, days, months, or even years, depending on the active agent present in each administration. Simultaneous administration refers to administration of two or more formulations at the same time or substantially at the same time (e.g., within seconds or just a few minutes apart), where the intent is that the formulations be administered together at the same time.

### Kits

Any of the compounds, compositions, formulations, particles, cells, etc. of the present invention described herein or a combination thereof can be presented as a combination kit. As used herein, the terms “combination kit” or “kit of parts” refers to the compounds, compositions, formulations, particles, and/or cells of the present invention and any additional components that are used to package, sell, market, deliver, and/or administer the combination of elements or a single element, such as the active ingredient, contained therein. Such additional components include, but are not limited to, packaging, syringes, blister packages, bottles, and the like. The kits can also contain additional reagents such as buffers, media, preservatives, and/or the like that can be appropriate for the preparation of and/or for use with the compounds, compositions, formulations, particles, and/or cells of the present invention. When one or more of the compounds, compositions, formulations, particles, and/or cells, described herein or a combination thereof (e.g., agents) contained in the kit are administered simultaneously, the combination kit can contain the active agents in a single formulation, such as a pharmaceutical formulation, (e.g., a tablet) or in separate formulations. When the compounds, compositions, formulations, particles, and/or cells described herein or a combination thereof and/or kit components are not administered simultaneously, the combination kit can contain each agent or other component in separate pharmaceutical formulations. The separate kit components can be contained in a single package or in separate packages within the kit.

In some embodiments, the combination kit also includes instructions printed on or otherwise contained in a tangible medium of expression. The instructions can provide information regarding the content of the compounds, compositions, formulations, particles, and/or cells, described herein or a combination thereof contained therein, safety information regarding the content of the compounds, compositions, formulations (e.g., pharmaceutical formulations), particles, and/or cells described herein or a combination thereof contained therein, information regarding the dosages, indications for use, and/or recommended treatment regimen(s) for the compound(s) and/or pharmaceutical formulations contained therein. In some embodiments, the instructions can provide directions for administering the compounds, compositions, formulations, particles, and cells described herein or a combination thereof to a subject in need thereof. In some embodiments, the subject in need thereof is in need of a reduction in the amount of one or more target proteins. In some embodiments, the subject in need thereof is in need of a treatment for or prevention of a cancer.

### Methods of Use

The engineered bifunctional receptors of the present invention can be used to degrade one or more target polypeptides. Thus, the engineered bifunctional receptors of the present invention have broad applicability to inter alia, treat diseases and disorders, improve existing therapies, and improve biomanufacturing.

Generally, methods of using the engineered bifunctional receptors include delivering or expressing one or more engineered bifunctional receptors in a cell or other environment where target polypeptide(s) are present under conditions in which the target polypeptide(s) can bind the target binding domain and an E3 ligase can bind the E3 ligase binding domain. Binding of the target polypeptide and E3 ligase by the engineered bifunctional receptor places the target polypeptide in proximity to the E3 ligase and/or complex thereby facilitating induced proximity degradation of the target polypeptide by the ubiquitin pathway.

In some embodiments, a method of targeted protein degradation includes delivering an engineered bifunctional receptor of the present invention, an engineered polynucleotide of the present invention, the vector or vector system of the present invention, the delivery vehicle of the present invention, a pharmaceutical formulation of the present invention, or any combination thereof to a cell or cell population under conditions sufficient to permit binding of the target binding domain to one or more target polypeptides thereby triggering induced-proximity degradation of the one or more target polypeptides. In some embodiments, the cell or cell population has a decreased amount of the one or more target polypeptides as compared to a suitable control cell. A “suitable control” is a control that will be instantly appreciated by one of ordinary skill in the art as one that is included such that it can be determined if the variable being evaluated had an effect, such as a desired effect or hypothesized effect. One of ordinary skill in the art will also instantly appreciate based on inter alia, the context, the variable(s), the desired or hypothesized effect, what is a suitable or an appropriate control needed. In some embodiments, delivering occurs in vitro, in vivo, in situ, or ex vivo. Delivery can occur one or more times (e.g., 1-25 or more) over a period of time. In some embodiments, the period of time ranges from 1 hour to 12 months or more. In some embodiments, the degrading one or more target peptides treats and/or prevents a disease, disorder, condition, or a symptom thereof.

In some embodiments, the amount of the target polypeptide in the cell to which an engineered bifunctional receptor is delivered or in which the engineered bifunctional receptor is expressed is reduced is by 0.001 fold to 1000 fold or more. In some embodiments, the amount of target polypeptide(s) is reduced to below the detection limit of a suitable detection technique.

In some embodiments, the cell or cell population is for a cell therapy. Exemplary cells and cells for cell therapy are described in greater detail elsewhere herein. In some embodiments, the cell or cell population is a cell for adoptive cell therapy, optionally a chimeric antigen receptor (CAR) T cell, T cell receptor (TCR) T cells, tumor infiltrating lymphocyte, B-cell, or a Natural Killer cell.

In some embodiments, the one or more target polypeptides are endogenous to the cell or cell population. In some embodiments, the one or more target polypeptides are exogenous to the cell or cell population. In some embodiments, the one or more target polypeptides are pathogenic or toxic to the cell or cell population.

In some embodiments, a method of treatment includes delivering a cell or cell population of the present invention, such as one that contains or expresses an engineered bifunctional receptor of the present invention, to a subject in need thereof. In some embodiments, the cell is autologous or allogeneic. Delivery can occur one or more times (e.g., 1-25 or more) over a period of time. In some embodiments, the period of time ranges from 1 hour to 12 months or more.

In some embodiments, the method further includes delivering an engineered bifunctional receptor of the present invention, an engineered polynucleotide of the present invention, a vector or vector system of the present invention, a delivery vehicle of the present invention, a pharmaceutical formulation of the present invention, or any combination thereof to the cell or cell population under conditions sufficient to permit binding of the target binding domain to one or more target polypeptides thereby triggering induced-proximity degradation of the one or more target polypeptides. In some embodiments, delivering occurs ex vivo. In some embodiments, the one or more target polypeptides are endogenous to the cell or cell population. In some embodiments, the one or more target polypeptides mediate cellular dysfunction, exhaustion, transformation, or any combination thereof. In some embodiments, the cell or cell population has a decreased amount of the one or more target polypeptides as compared to a suitable control cell. In some embodiments, the amount of the one or more target polypeptides is decreased by 0.001 to 1,000 fold or more. In some embodiments, the amount of the one or more target polypeptides is decreased such that they are not detectable by a conventional method.

In some embodiments, the subject in need thereof has a cancer. In some embodiments, the cancer is a solid tumor. In some embodiments, the cancer is not a solid-tumor cancer. As used herein “cancer” refers to one or more types of cancer including, but not limited to, acute lymphoblastic leukemia, acute myeloid leukemia, adrenocortical carcinoma, Kaposi Sarcoma, AIDS-related lymphoma, primary central nervous system (CNS) lymphoma, anal cancer, appendix cancer, astrocytomas, atypical teratoid/Rhabdoid tumors, basal cell carcinoma of the skin, bile duct cancer, bladder cancer, bone cancer (including but not limited to Ewing Sarcoma, osteosarcomas, and malignant fibrous histiocytoma), brain tumors, breast cancer, bronchial tumors, Burkitt lymphoma, carcinoid tumor, cardiac tumors, germ cell tumors, embryonal tumors, cervical cancer, cholangiocarcinoma, chordoma, chronic lymphocytic leukemia, chronic myelogenous leukemia, chronic myeloproliferative neoplasms, colorectal cancer, craniopharyngioma, cutaneous T-Cell lymphoma, ductal carcinoma in situ, endometrial cancer, ependymoma, esophageal cancer, esthesioneuroblastoma, extracranial germ cell tumor, extragonadal germ cell tumor, eye cancer (including, but not limited to, intraocular melanoma and retinoblastoma), fallopian tube cancer, gallbladder cancer, gastric cancer, gastrointestinal carcinoid tumor, gastrointestinal stromal tumors, central nervous system germ cell tumors, extracranial germ cell tumors, extragonadal germ cell tumors, ovarian germ cell tumors, testicular cancer, gestational trophoblastic disease, Hairy cell leukemia, head and neck cancers, hepatocellular (liver) cancer, Langerhans cell histiocytosis, Hodgkin lymphoma, hypopharyngeal cancer, islet cell tumors, pancreatic neuroendocrine tumors, kidney (renal cell) cancer, laryngeal cancer, leukemia, lip cancer, oral cancer, lung cancer (non-small cell and small cell), lymphoma, melanoma, Merkel cell carcinoma, mesothelioma, metastatic squamous cell neck cancer, midline tract carcinoma with and without NUT gene changes, multiple endocrine neoplasia syndromes, multiple myeloma, plasma cell neoplasms, mycosis fungoides, myelodysplastic syndromes, myelodysplastic/myeloproliferative neoplasms, chronic myelogenous leukemia, nasal cancer, sinus cancer, non-Hodgkin lymphoma, pancreatic cancer, paraganglioma, paranasal sinus cancer, parathyroid cancer, penile cancer, pharyngeal cancer, pheochromocytoma, pituitary cancer, peritoneal cancer, prostate cancer, rectal cancer, Rhabdomyosarcoma, salivary gland cancer, uterine sarcoma, Sezary syndrome, skin cancer, small intestine cancer, large intestine cancer (colon cancer), soft tissue sarcoma, T-cell lymphoma, throat cancer, oropharyngeal cancer, nasopharyngeal cancer, hypopharyngeal cancer, thymoma, thymic carcinoma, thyroid cancer, transitional cell cancer of the renal pelvis and ureter, urethral cancer, uterine cancer, vaginal cancer, cervical cancer, vascular tumors and cancer, vulvar cancer, and Wilms Tumor.

In some embodiments of a method, an amount of thalidomide or a derivative thereof is delivered to a cell or cell population that is or will be expressing or containing an engineered bifunctional receptor of the present invention. Exemplary thalidomide derivatives are described in greater detail elsewhere herein. In some embodiments, a stimulus is applied one or more times to a cell or cell population that is or will be expressing or containing an engineered bifunctional receptor of the present invention.

Further embodiments are illustrated in the following Examples which are given for illustrative purposes only and are not intended to limit the scope of the invention.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure. The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

### Example 1—Engineered Bifunctional Receptors

This Example describes and demonstrates the design, generation, and function of exemplary engineered bifunctional receptors and uses thereof.

### Materials and Methods

Vector cloning: Amino acid sequences of human gene fragments were downloaded using Uniprot. Nucleotide sequences were then generated with codon optimization software and produced by double stranded DNA synthesis. Entry vectors were linearized by restriction digest with BsmBI. DNA fragments were introduced by Gibson Assembly or T4 ligation. Vector sequences were confirmed by Sanger sequencing. Reporters of target protein abundance were generated by cloning into “Cilantro2” or “Chive” vectors, fusing these sequences in frame with GFP, either at the N or C terminus, followed by an IRES-mCherry element to identify the transduced cells. SSRs were fused into lentivector pJL002 in which the SSR was followed by a P2A cleavage sequence and mTagBFP2 as a marker gene. When incorporated into CARs, SSRs were cloned upstream of an anti-CD19 scFv—CD8 hinge/transmembrane—4-1BB costimulatory domain—CD3z ITAM-containing domain CAR lentivector, pJL001.

### Jurkat Transduction and Functional Assays.

Lentiviral particles were produced in HEK 293T cells with a second or third generation production system. Lentiviral particles were used directly from supernatant or concentrated by ultracentrifugation. Jurkat cells were subsequently transduced.

### Preliminary SSR Optimization.

Overexpression of the target protein-GFP fusion was used as a preliminary screen to validate each SSR. Lentiviruses were produced in HEK293T cells. Then, Jurkat or HEK293T cells were co-transduced with the target protein reporter lentivirus and the SSR lentivirus. For lenalidomide-inducible systems, the drug was added one day before analysis unless otherwise noted. Cells were analyzed by flow cytometry: co-transduced cells (mCherry+mtagBFP2+) and target only cells (mCherry+mtagBFP2−) were assessed for relative GFP abundance to indicate SSR-dependent target protein depletion.

To validate endogenous protein degradation, Jurkat cells were transduced with the SSR lentivirus. For the DNMT3A degrader, the transduced mtagBFP2+ cells were enriched by fluorescence-activated cell sorting, lysed, and sonicated or treated with benzonase to disrupt chromatin. Protein lysates were analyzed by Western blot for DNMT3A and actin as a loading control. For the SMAD degrader, after lentiviral transduction the cells were analyzed by intracellular flow cytometry for SMAD2/3 abundance. To assess SMAD transcriptional responses, TGF-beta HEKBLUE cells were transduced with control or the SMAD degrader, then plated and analyzed per manufacturer's directions with or without 10 ng/mL recombinant TGF-beta.

### Protein Analysis (Western).

Cells were frozen as cell pellets. Cells were lysed in RIPA buffer containing HALT protease/phosphatase. Cell lysates were treated with benzonase or sonication for DNA fragmentation. Lysis protein levels were quantified and normalized. DNMT3A detected with rabbit monoclonal D23G1 (Cell Signaling Technology). Actin detected with antibody #3700 (Cell Signaling Technology).

To assess the effect of DNMT3A and SMAD degraders on CAR T cell function, we cloned control CAR lentivectors or multicistronic degrader CAR lentivectors, all CARs targeting CD19. Primary human T cells were activated, dynabead stimulated, and transduced. After 2 weeks of expansion in vitro, the cells were then used in functional assays. To assess for short-term cytotoxicity, CAR T cells were co-incubated with NALM6-GFP-luciferase tumor cells overnight, followed by quantification of luminescence. To assess for medium-term cytotoxicity and T cell proliferation, CAR T cells were co-incubated with NALM6-GFP-luciferase tumor cells for 5 days and monitored by live-cell imaging. This enabled assessment of the dynamics of tumor cell killing and T cell proliferation. To assess for long-term in vitro growth, CAR T cells were repetitively stimulated each week for one month with irradiated K562-CD19 cells, which lack MHC expression. After each week, CAR T cell growth was measured, and the phenotype of the T cells was evaluated by flow cytometry, using a panel of antibodies for CD3, CD4, CD8, CD45RA, CCR7, TIM3, LAG3, and PD-1.

### Primary Human T Cell Culture and Transduction.

Primary human T cells were thawed and cultured for 14 days in media with IL2, with CD3/28 bead stimulation for the first seven days. T cells were transduced on Day 1 using ultracentrifuge-concentrated lentivirus to a target MOI of 5.

### CAR T Cell Coculture Assays.

Primary human CAR T cells were cocultured with NALM6 target cells engineered to express GFP and luciferase at indicated ratios for 4-6 days. Cells were imaged every hour using the Incucyte Zoom imaging system and analyzed for mCherry and GFP area. Cells were also cocultured in a separate overnight assay at defined target-to-T cell ratios, after which the cellular luciferase was quantified as a measure of target cell cytotoxicity. Primary human CAR T cells were repetitively stimulated with irradiated CD19+K562 cells at a 1:1 ratio each week for 3 or 4 weeks.

### CAR T Cell Immunophenotyping.

CAR T cells were harvested from coculture assays and stained using a 9-antibody phenotype panel (Table 5). Cells were then analyzed via flow cytometry.

TABLE 5

| Ab | [ ] | Vendor | | Cat # |
|---|---|---|---|---|
| FITC-CCR7 | 1:25 | BD | 150503 | 561271 |
| BV786 PD1 | 1:33 | BD | EH12.1 | 563789 |
| BV711 TIM3 | 1:50 | BD | 7D3 | 565566 |
| AF647 LAG3 | 1:50 | BD | T47-530 | 565716 |
| PeCy7 CD45RA | 1:50 | Biolegend | HI100 | 304126 |
| V500 CD8 | 1:50 | BD | SK1 | 647458 |
| V450 CD4 | 1:50 | BD | SK3 | 651850 |
| APCH7 CD3 | 1:50 | BD | SK7 | 641406 |
| DAPI | 1:100 | Biolegend | | 422801 |
| BV Stain buffer | | BD | | 566349 |

### Statistical Analysis.

Two-sided student's t-tests or two-sided ANOVA tests were performed.

### Results

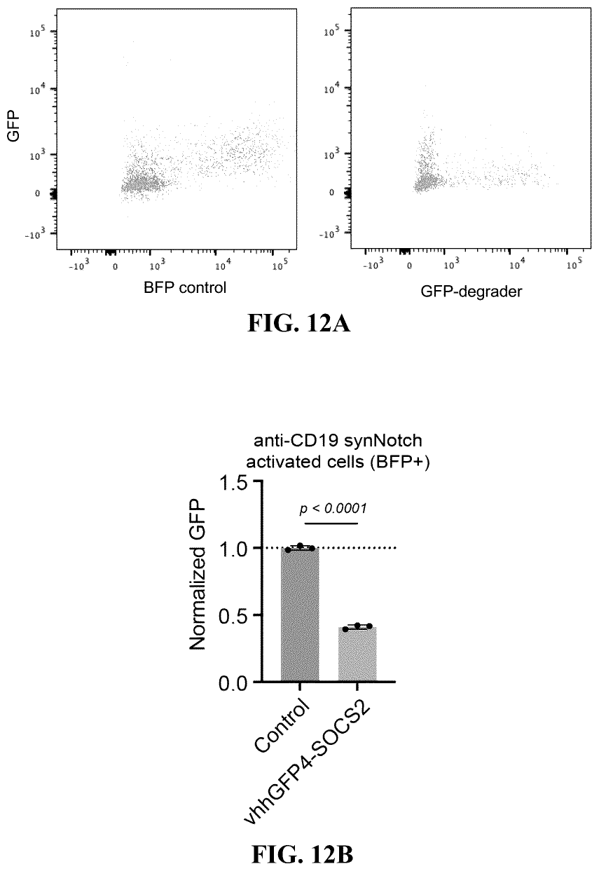

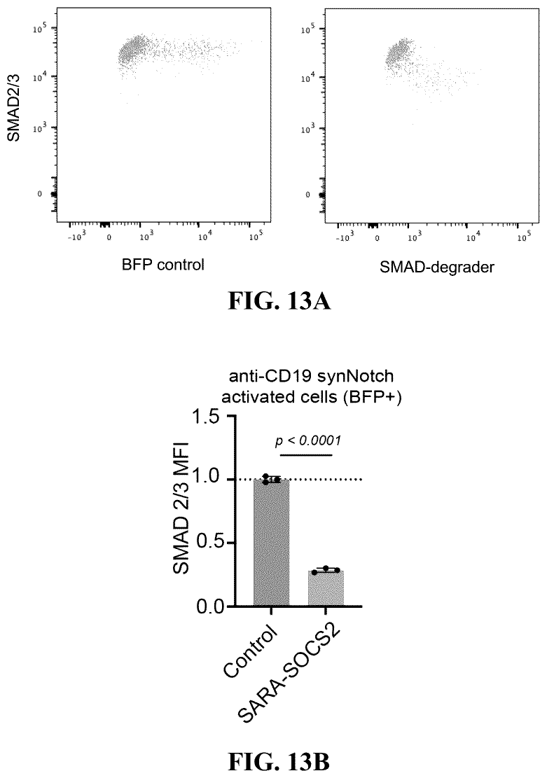

FIG. 1A-1H shows reprogramming E3 ligase specificity using synthetic substrate receptors (SSRs). FIG. 1A shows a schematic of the degradation system using a GFP-binding SSR. FIG. 1B shows normalized GFP abundance in HEK293T cells dually transduced with GFP and a GFP degrader composed of the vhhGFP4 nanobody linked to the indicated ligase-binding domain. FIG. 1C shows GFP intensity in Jurkat cells transduced with GFP and/or the GFP degrader vhhGFP4-SOCS2. FIG. 0.1D shows normalized GFP abundance in Jurkat cells treated with the neddylation inhibitor MLN4924. Cells were treated with 0.5 uM MLN4924 for 4 hours and analyzed by flow cytometry. FIG. 1E shows normalized GFP abundance in multiple cell lines transduced with GFP and either the SOCS2- or Vpx-based GFP degrader. FIG. 1F shows normalized abundance of untagged GFP in the nucleus/cytosol and cell surface-localized GFP fused to a type I transmembrane domain of Jurkat cells co-transduced with the SOCS2-based GFP degrader. FIGS. 1G and 11 show a schematic of a cell activation-inducible degradation system using a CD19-activated SynNotch receptor. FIGS. 1H, 12B, and 13B show normalized GFP abundance (FIGS. 1H and 12B) or SMAD 2/3 mean fluorescent intensity (MFI) (FIG. 13B) in Jurkat cells transduced to express GFP or SMAD 2/3 and either a control SynNotch receptor or a SynNotch-degrader construct (vhhGFP4-SOCS2 or SARA-SOCS2). Jurkat cells were stimulated with CD19+ target cells overnight and analyzed via flow cytometry (FIGS. 12A and 13A). Two-sided student's t-tests were performed as indicated. Experiments were performed in technical duplicate; data represents one of two independent experiments.

FIG. 2A-2E shows engineering a lenalidomide-inducible SSR system. (FIG. 2A) Schematic of the lenalidomide-inducible degradation system using a GFP-binding SSR and a zinc finger ligase-binding domain. FIG. 2B shows GFP intensity in Jurkat cells dually transduced with GFP and the lenalidomide-inducible GFP degrader ZnF-vhhGFP4. Cells were incubated with or without 1000 nM lenalidomide for two hours and analyzed via flow cytometry. (FIG. 2C) Normalized GFP abundance in Jurkat cells dually transduced with GFP and the lenalidomide-inducible GFP degrader. FIG. 2D shows a timecourse assay showing normalized GFP degradation in Jurkat cells dually transduced with GFP and a GFP-targeted SSR and treated with 1000 nM lenalidomide. (FIG. 2E) Dose response assay showing normalized GFP degradation in dually transduced Jurkat cells treated overnight with 1000 nM lenalidomide.

FIG. 3A-3K shows targeted degradation of cell therapy-relevant endogenous proteins. FIG. 3A shows a schematic of the degradation system using a SARA-based SSR to bind SMAD2/3. FIG. 3B shows a schematic of SMAD2/3-dependent signaling in the TGFβ pathway. FIG. 3C shows hnormalized GFP abundance in Jurkat cells dually transduced with a SMAD2- or SMAD3-GFP reporter and SOCS2-based or zinc finger lenalidomide-inducible degrader. ZnF-SARA Jurkat cells were incubated overnight with or without 1000 nM lenalidomide. FIG. 3D is a flow plot showing GFP and mTagBFP intensity in Jurkat cells dually transduced with either a SMAD2- or SMAD3-GFP reporter and a SARA-based degrader. FIG. 3E shows Endogenous SMAD2/3 intensity in Jurkat cells transduced with the SARA-based degrader. Cells were analyzed via intracellular flow (ICF). FIG. 3F shows mean fluorescent intensity (MFI) of SMAD2/3 in Jurkat cells transduced with the SARA-based degrader. FIG. 3G shows normalized levels of SMAD reporter in HEKBlue cells transduced with the SARA-based degrader. FIG. 3H shows a schematic of the degradation system using a DNMT3L-based SSR to degrade DNMT3A. FIG. 3I is a Flow plot showing GFP and mTagBFP intensity in Jurkat cells dually transduced with either a DNMT3A- or DNMT3B-GFP reporter and a DNMT3L-based degrader. FIG. 3J shows normalized GFP in Jurkat cells dually transduced with a DNMT3A-GFP reporter and either a SOCS2-based or lenalidomide-inducible zinc finger-based degrader. FIG. 3K shows exemplary Western blot results showing endogenous DNMT3A degradation in Jurkat cells transduced with a DNMT3L-based degrader containing a variable nuclear localization signal.

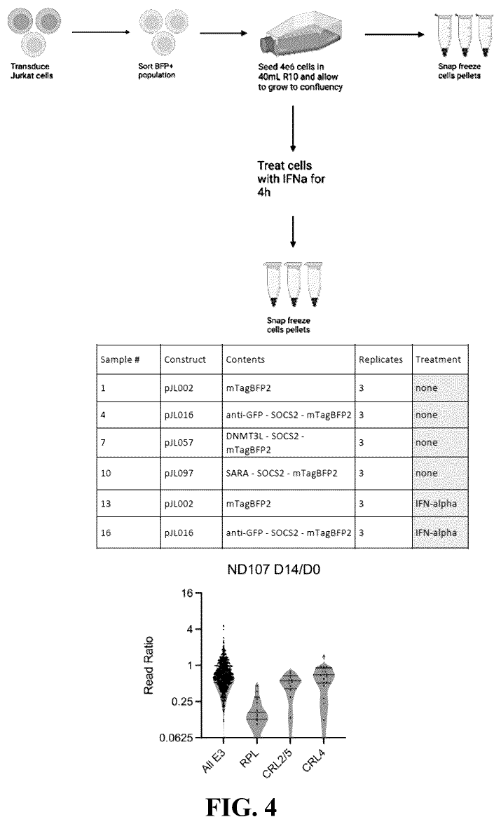

FIG. 4 shows a strategy and results of an exemplary proteomic and CRISPR-based screen for characterizing and analyzing specificity of an engineered bifunctional receptor described herein. FIG. 4 shows an example that tests the specificity of SMAD and DNMT3A targeting engineered bifunctional receptors.

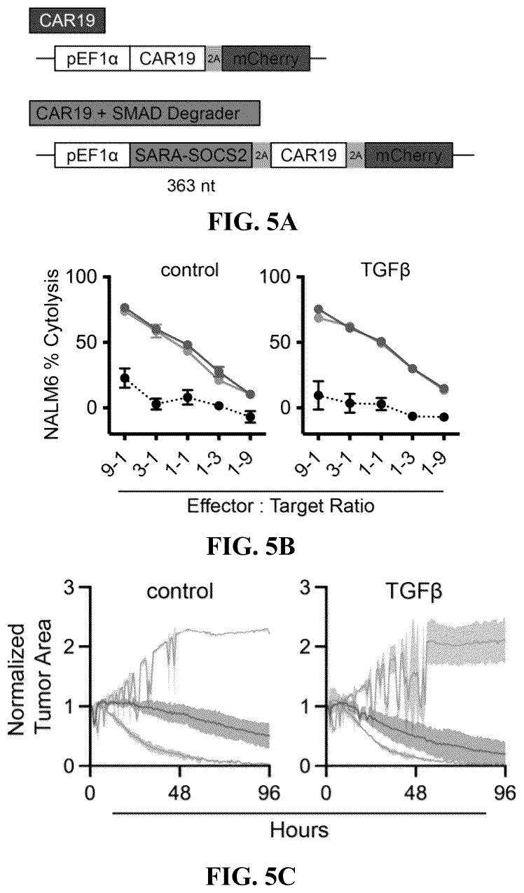

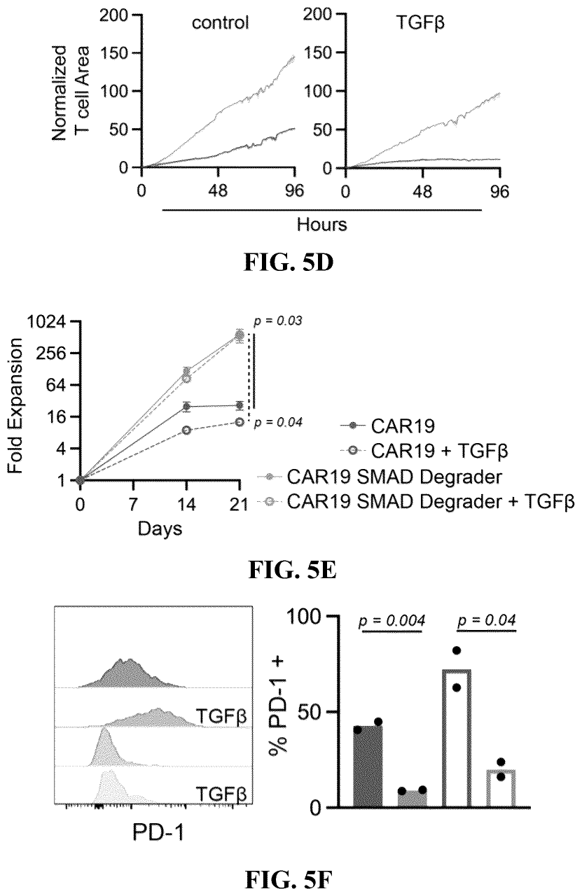

FIG. 5A-5F shows SMAD degradation enhanced CAR T cell proliferation and anti-tumor function. FIG. 5A shows the design of a conventional CD19-targeted CAR and a SMAD degrader-containing CD19 CAR. FIG. 5B shows percent cytolysis of NALM6 tumor cells in a coculture assay with either CAR19 or CAR19+SMAD degrader T cells. CAR T cells and tumor cells were cocultured overnight at various ratios, with or without [conc] TGFb. FIG. 5C shows normalized tumor area in an incucyte coculture assay. CAR19 or CAR19+SMAD degrader T cells were cultured at a 1:1 ratio with NALM6 tumor cells with or without exogenous TGFb for five days and imaged every hour. FIG. 5D shows normalized T cell area in an incucyte coculture assay. FIG. 5E shows CAR T cell expansion in a long term proliferation coculture assay. CAR19 or CAR19+SMAD degrader T cells were cultured with CD19+K562 tumor cells for three weeks, with weekly restimulation. FIG. 5F shows PD-1 abundance and % PD-1% of CD4+ CAR T cells.

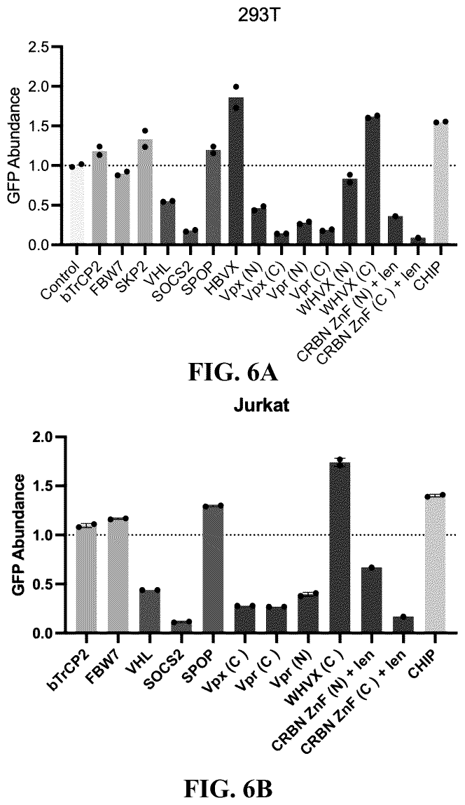

FIG. 6A-6B shows targeted degradation of GFP with multiple E3 ligase recruiting domains. FIG. 6A shows normalized GFP abundance in HEK293T cells dually transduced with GFP and a GFP degrader composed of the vhhGFP4 nanobody linked to the indicated ligase-binding domain. FIG. 6B normalized GFP abundance in Jurkat cells dually transduced with GFP and a GFP degrader composed of the vhhGFP4 nanobody linked to the indicated ligase-binding domain.

### Example 2—Engineering Vpx to Target and Degrade a Non-Native Target Protein

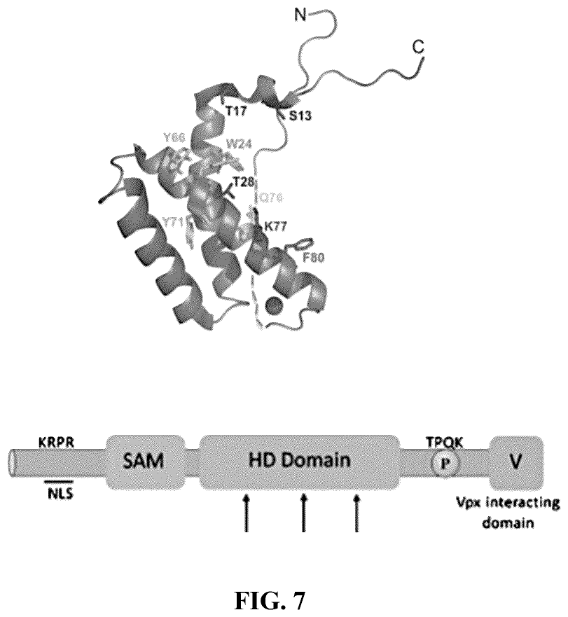

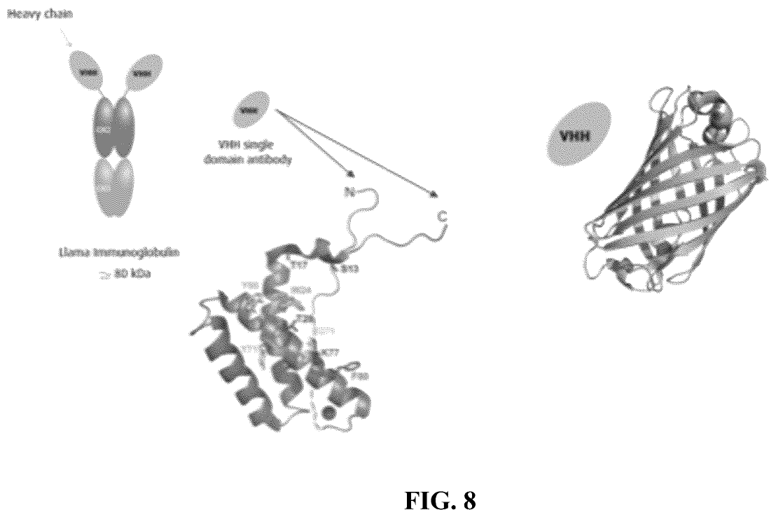

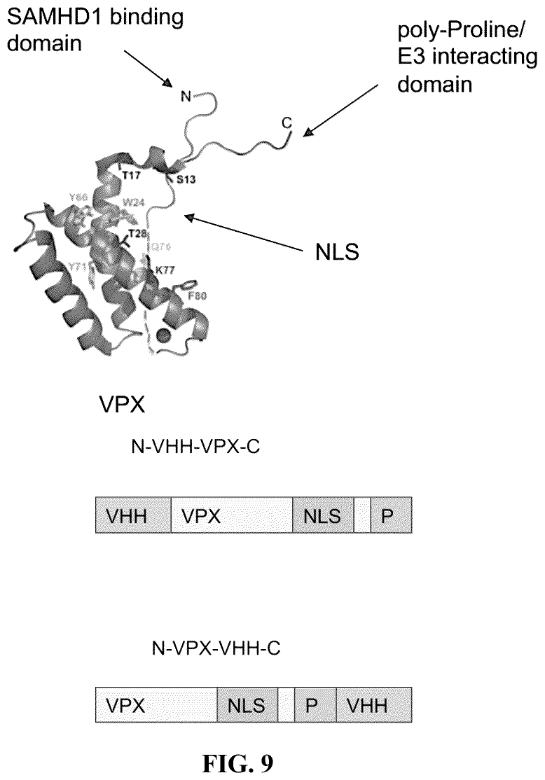

Viral protein X (Vpx) is an HIV derived protein having an E3 ligase recruiting domain and a recognition domain that natively targets anti-viral protein SAMHD1 (see, e.g., FIG. 7). This Example demonstrates reprogramming of Vpx to target a protein other than SAMHD1. In this proof-of-concept strategy, the Vpx was reprogrammed to target GFP. Briefly, an anti-GFP VHH single domain antibody was fused to a C- or N-terminus of a Vpx protein (see, e.g., FIGS. 8-9). VHHs tested and their origin is shown in Table 6.

TABLE 6
Anti-GFP VHHs and their Origin

| | Kd | | |
|---|---|---|---|
| VHH | (nM) | Sequence | Source |
| 9872 | 0.13 | | Sarens et al., J Mol Biol. |
| | | | 377(2):478-488 (2008). |
| 8766 | 1.2 | | https://doi.org/10.1016/ |
| | | | j.jmb.2008.01.022 |
| LaG16 | 0.7 | MAQVQLVESGGRLVQAGDSLRLSCAA | Fridy et al. Nat. Methods. |
| | | SGRTFSTSAMAWFRQAPGREREFVAAI | 2014. Dec; 11(12):1253- |
| | | TWTVGNTILGDSVKGRFTISRDRAKNT | 1260, particularly at the |
| | | VDLQMDNLEPEDTAVYYCSARSRGYV | Supplementary FIG. 4. |
| | | LSVLRSVDSYDYWGQGTQVTVS (SEQ | |
| | | ID NO: 288) | |
| LaG17 | 50 | MADVQLVESGGGLVQAGGSLRLSCAA | |
| | | SGRTISMAAMSWFRQAPGKEREFVAGI | |
| | | SRSAGSAVHADSVKGRFTISRDNTKNT | |
| | | LYLQMNSLKAEDTAVYYCAVRTSGFF | |
| | | GSIPRTGTAFDYWGQGTQVTVS (SEQ | |
| | | ID NO: 289) | |
| LaG18 | 3800 | MAQVQLVESGGGLVQTGGSLKLSCTA | |
| | | SVRTLSYYHVGWFRQAPGKEREFVAG | |
| | | IHRSGESTFYADSVKGRFTISRDNAKN | |
| | | TVHLQMNSLKPEDTAVYYCAQRVRGF | |
| | | FGPLRSTPSWYDYWGQGTQVTVS | |
| | | (SEQ ID NO: 290) | |
| LaG41 | 0.9 | MADVOLVESGGGLVQAGGSLRLSCAA | |
| | | SGPTGAMAWFRQAPGKEREFVGGISG | |
| | | SETDTYYVDSVKGRFTVDRDNVKNTV | |
| | | YLQMNSLKPEDTAVYYCAARRRITLFT | |
| | | SRTDYDFWGRGTQVTV (SEQ ID NO: | |
| | | 291) | |
| A12 | Unknown | MADVQLQESGGGLVQAGGSLRLSCAA | GenBank Accession No: |
| | | SGGTFSILSLGWFRQAPGKEREFVAAIS | MN813056.1 available at |
| | | RSEGSTDYADFVKGRFMISRENAKNT | https://www.ncbi.nlm.nih. |
| | | AYLQMNSLKPEDTAVYFCAASYARRL | gov/nuccore/MN813056.1 |
| | | STTASRVLYWGQGTQVTVSS (SEQ ID | |
| | | NO: 292) | |

### Methods

Briefly, stable HTEK293 cells expressing an NLS tag GFP (HEK293-nlsGFP) under control of a CMV promoter were generated using standard techniques. Several VHHs known to target GFP (Table 7) were cloned into a transfection vector and fused to Vpx at the N- or C-terminus of the Vpx. The Vpx used was derived from SIVmac239. Stable HEK-293 GFP expressing cells were then transfected with the Vpx-VHH constructs and analyzed for GFP expression 1-3 days post transfection of the Vpx-VHH constructs. Targeted degradation of the GFP by the reprogrammed Vpx protein was determined by measuring GFP signal (fluorescence) using a suitable technique (e.g., FACS). A decrease in GFP signal indicates successful targeting by the Vpx-VHH protein and subsequent degradation via the endogenous ubquination pathway.

TABLE 7
Sequences of the Vpx-VHH proteins

| | Sequence (Vpx is bolded, GSSSS3 linker is |
|---|---|
| Construct | italicized, VHH is underlined) |
| Vpx-A12 | MSDPRERIPPGNSGEETIGEAFEWLNRTVEEINREAVNHLPRELIFQVWQR |
| | SWEYWHDEQGMSPSYVKYRYLCLIQKALFMHCKKGCRCLGEGHGAGG |
| | WRPGPPPPPPPGLAGGGGSGGGGSGGGGSMADVQLQESGGGLVQAGGSLRL |
| | SCAASGGTFSILSLGWFRQAPGKEREFVAAISRSEGSTDYADFVKGRFMISREN |
| | AKNTAYLQMNSLKPEDTAVYFCAASYARRLSTTASRVLYWGQGTQVTVSS |
| | (SEQ ID NO: 293) |
| Vpx-Lag16 | MSDPRERIPPGNSGEETIGEAFEWLNRTVEEINREAVNHLPRELIFQVWQR |
| | SWEYWHDEQGMSPSYVKYRYLCLIQKALFMHCKKGCRCLGEGHGAGG |
| | WRPGPPPPPPPGLAGGGGSGGGGSGGGGSMAQVQLVESGGRLVQAGDSLRLS |
| | CAASGRTFSTSAMAWFRQAPGREREFVAAITWTVGNTILGDSVKGRFTISRDRA |
| | KNTVDLQMDNLEPEDTAVYYCSARSRGYVLSVLRSVDSYDYWGQGTQVTVS |
| | (SEQ ID NO: 294) |
| A12-Vpx | MADVQLQESGGGLVQAGGSLRLSCAASGGTFSILSLGWFRQAPGKEREFVAAI |
| | SRSEGSTDYADFVKGRFMISRENAKNTAYLQMNSLKPEDTAVYFCAASYARRL |
| | STTASRVLYWGQGTQVTVSSGGGGSGGGGSGGGGSMSDPRERIPPGNSGEETI |
| | GEAFEWLNRTVEEINREAVNHLPRELIFQVWQRSWEYWHDEQGMSPSYV |
| | KYRYLCLIQKALFMHCKKGCRCLGEGHGAGGWRPGPPPPPPPGLA (SEQ |
| | ID NO: 295) |
| Lag16-Vpx | MAQVOLVESGGRLVQAGDSLRLSCAASGRTFSTSAMAWFRQAPGREREFVAA |
| | ITWTVGNTILGDSVKGRFTISRDRAKNTVDLQMDNLEPEDTAVYYCSARSRGY |
| | VLSVLRSVDSYDYWGQGTQVTVSGGGGSGGGGSGGGGSMSDPRERIPPGNS |
| | GEETIGEAFEWLNRTVEEINREAVNHLPRELIFQVWQRSWEYWHDEQGM |
| | SPSYVKYRYLCLIQKALFMHCKKGCRCLGEGHGAGGWRPGPPPPPPPGLA |
| | (SEQ ID NO: 296) |

### Results

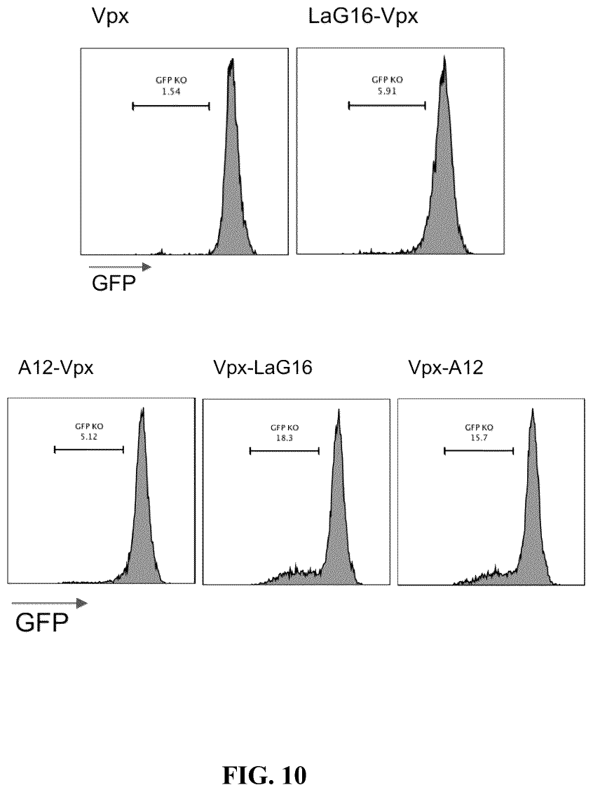

Results are shown in FIG. 10. The C-terminus of the Vpx-VHH protein contains an E3 ubiquitin ligase recruiting domain. High affinity nanobodies were capable of targeting and subsequent degradation. Vpx has an NLS so it was restricted to degrading nuclear GFP in this Example. However, it is expected, based on the results demonstrated in this Example, that removal of the NLS or transient co-transfection of both the Vpx-VHH construct and GFP would allow for degradation of cytosolic GFP. Successful targeting and degradation by constructs with VHHs from independent sources suggest that the targeting and degradation of the target GFP protein was independent of the epitope.

### Example 3—Construct Sequences Used in Examples 1 and 2

Tables 8-9 shows sequences of the constructs and components thereof used in Examples 1 and 2.

TABLE 8

| Construct | | | |
|---|---|---|---|
| name | Insert | Insert nucleotide sequences | Insert amino acid sequence |
| pJL012 | CAR19 | GAAGTCAAGCTTCAGGAGTCAGGACCCGGTCTTGT | MALPVTALLLPLALLLHAARPEQKLISEED |
| | | AGCTCCCAGCCAAAGCCTGTCAGTTACATGCACGG | LDIQMTQTTSSLSASLGDRVTISCRASQDIS |
| | | TTTCCGGTGTGTCTTTGCCAGATTATGGCGTATCTT | KYLNWYQQKPDGTVKLLIYHTSRLHSGVP |
| | | GGATTCGCCAACCGCCTAGAAAGGGACTTGAGTG | SRFSGSGSGTDYSLTISNLEQEDIATYFCQQ |
| | | GTTGGGTGTCATTTGGGGATCAGAAACAACTTACT | GNTLPYTFGGGTKLEITGSTSGSGKPGSGE |
| | | ATAACAGTGCTCTTAAGTCCAGGTTGACTATAATC | GSTKGEVKLQESGPGLVAPSQSLSVTCTVS |
| | | AAGGACAATAGTAAGTCCCAAGTTTTTCTGAAAAT | GVSLPDYGVSWIRQPPRKGLEWLGVIWGS |
| | | GAATTCCCTGCAGACAGATGACACCGCTATCTACT | ETTYYNSALKSRLTIIKDNSKSQVFLKMNS |
| | | ACTGTGCCAAGCACTACTATTATGGGGGCTCTTAT | LQTDDTAIYYCAKHYYYGGSYAMDYWG |
| | | GCTATGGACTATTGGGGTCAGGGGACATCAGTTAC | QGTSVTVSS (SEQ ID NO: 2) |
| | | TGTTTCCAGC (SEQ ID NO: 1) | |
| pJL_013 | vhhGFP4- | ATGggaggccttGATTACAAGGACGACGACGACAAGgc | MGGLDYKDDDDKASDQVQLVESGGALV |
| | 913iK0 | tagcGATCAAGTCCAACTGGTGGAGTCTGGTGGCGC | QPGGSLRLSCAASGFPVNRYSMRWYRQA |
| | | TTTGGTGCAGCCAGGTGGCTCTCTGCGTTTGTCCT | PGKEREWVAGMSSAGDRSSYEDSVKGRF |
| | | GTGCCGCTTCTGGCTTCCCAGTGAACCGCTATTCC | TISRDDARNTVYLQMNSLKPEDTAVYYCN |
| | | ATGCGCTGGTATCGCCAGGCTCCAGGCAAAGAGC | VNVGFEYWGQGTQVTVSSGSGSGSGFNV |
| | | GTGAGTGGGTAGCCGGTATGTCCAGCGCGGGTGA | LMVHRRSHTGERPLQCEICGFTCRQRGNL |
| | | TCGTAGCTCCTATGAAGACTCCGTGAAGGGCCGTT | LRHIRLHTGERPFRCHLCNYACQRRDAL |
| | | TCACCATCAGCCGTGACGATGCCCGTAACACGGTG | (SEQ ID NO: 4) |
| | | TATCTGCAAATGAACAGCTTGAAACCTGAAGATA | |
| | | CGGCCGTGTATTACTGTAATGTGAACGTGGGCTTC | |
| | | GAGTATTGGGGCCAAGGCACCCAGGTCACCGTgTC | |
| | | CAGCGGCAGTGGTAGTGGCtccggaTTCAATGTCTTA | |
| | | ATGGTTCATcggCGAAGCCATACTGGTGAACGCCC | |
| | | ATTGCAGTGCGAAATATGCGGCTTTACCTGCCGCC | |
| | | AGcgcGGTAACCTCCTCCGCCACATTcgtCTGCACAC | |
| | | AGGGGAAcggCCTTTTcggTGTCACCTCTGCAACTAT | |
| | | GCATGCCAAAGAAGAGATGCGCTC (SEQ ID NO: 3) | |
| pJL_014 | vhhGFP4- | ATGggaggccttGATTACAAGGACGACGACGACAAGgc | MGGLDYKDDDDKASDQVQLVESGGALV |
| | CHIP | tagcGATCAAGTCCAACTGGTGGAGTCTGGTGGCGC | QPGGSLRLSCAASGFPVNRYSMRWYRQA |
| | | TTTGGTGCAGCCAGGTGGCTCTCTGCGTTTGTCCT | PGKEREWVAGMSSAGDRSSYEDSVKGRF |
| | | GTGCCGCTTCTGGCTTCCCAGTGAACCGCTATTCC | TISRDDARNTVYLQMNSLKPEDTAVYYCN |
| | | ATGCGCTGGTATCGCCAGGCTCCAGGCAAAGAGC | VNVGFEYWGQGTQVTVSSGSGSGSGRLN |
| | | GTGAGTGGGTAGCCGGTATGTCCAGCGCGGGTGA | FGDDIPSALRIAKKKRWNSIEERRIHQESEL |
| | | TCGTAGCTCCTATGAAGACTCCGTGAAGGGCCGTT | HSYLSRLIAAERERELEECQRNHEGDEDDS |
| | | TCACCATCAGCCGTGACGATGCCCGTAACACGGTG | HVRAQQACIEAKHDKYMADMDELFSQVD |
| | | TATCTGCAAATGAACAGCTTGAAACCTGAAGATA | EKRKKRDIPDYLCGKISFELMREPCITPSGI |
| | | CGGCCGTGTATTACTGTAATGTGAACGTGGGCTTC | TYDRKDIEEHLQRVGHFDPVTRSPLTQEQ |
| | | GAGTATTGGGGCCAAGGCACCCAGGTCACCGTgTC | LIPNLAMKEVIDAFISENGWVEDY (SEQ |
| | | CAGCGGCAGTGGTAGTGGCtccggaAGGCTCAATTTT | ID NO: 6) |
| | | GGGGACGATATACCATCAGCACTCCGCATAGCCA | |
| | | AGAAAAAAAGATGGAACTCAATCGAGGAAAGGA | |
| | | GAATCCATCAGGAATCTGAACTTCATAGCTATTTG | |
| | | TCCCGACTGATTGCAGCAGAGAGAGAAAGGGAGC | |
| | | TTGAGGAATGTCAACGCAATCACGAAGGGGACGA | |
| | | GGATGATTCTCACGTACGCGCACAACAAGCCTGTA | |
| | | TCGAGGCCAAACATGATAAATATATGGCAGATAT | |
| | | GGACGAACTGTTTTCCCAGGTGGACGAGAAACGC | |
| | | AAGAAAAGAGATATTCCTGACTATCTCTGTGGCAA | |
| | | GATCAGTTTTGAATTGATGCGCGAACCATGTATAA | |
| | | CCCCCAGTGGCATTACTTATGACCGAAAGGATATT | |
| | | GAAGAACATCTGCAACGGGTGGGGCACTTTGATC | |
| | | CAGTTACACGGTCTCCGCTCACCCAGGAACAGCTT | |
| | | ATTCCAAACTTGGCCATGAAAGAGGTAATAGACG | |
| | | CCTTTATTAGCGAGAACGGATGGGTCGAGGACTA | |
| | | C (SEQ ID NO: 5) | |
| pJL_015 | vhhGFP4- | ATGggaggccttGATTACAAGGACGACGACGACAAGgc | MGGLDYKDDDDKASDQVQLVESGGALV |
| | HBVX | tagcGATCAAGTCCAACTGGTGGAGTCTGGTGGCGC | QPGGSLRLSCAASGFPVNRYSMRWYRQA |
| | | TTTGGTGCAGCCAGGTGGCTCTCTGCGTTTGTCCT | PGKEREWVAGMSSAGDRSSYEDSVKGRF |
| | | GTGCCGCTTCTGGCTTCCCAGTGAACCGCTATTCC | TISRDDARNTVYLQMNSLKPEDTAVYYCN |
| | | ATGCGCTGGTATCGCCAGGCTCCAGGCAAAGAGC | VNVGFEYWGQGTQVTVSSGSGSGSGNAH |
| | | GTGAGTGGGTAGCCGGTATGTCCAGCGCGGGTGA | QILPKVLHKRTLGLS (SEQ ID NO: 8) |
| | | TCGTAGCTCCTATGAAGACTCCGTGAAGGGCCGTT | |
| | | TCACCATCAGCCGTGACGATGCCCGTAACACGGTG | |
| | | TATCTGCAAATGAACAGCTTGAAACCTGAAGATA | |
| | | CGGCCGTGTATTACTGTAATGTGAACGTGGGCTTC | |
| | | GAGTATTGGGGCCAAGGCACCCAGGTCACCGTgTC | |
| | | CAGCGGCAGTGGTAGTGGCtccggaAACGCGCATCA | |
| | | AATATTGCCTAAGGTCCTGCATAAACGAACCTTGG | |
| | | GTCTTTCC (SEQ ID NO: 7) | |
| pJL_016 | vhhGFP4- | ATGggaggccttGATTACAAGGACGACGACGACAAGgc | MGGLDYKDDDDKASDQVQLVESGGALV |
| | SOCS2 | tagcGATCAAGTCCAACTGGTGGAGTCTGGTGGCGC | QPGGSLRLSCAASGFPVNRYSMRWYRQA |
| | | TTTGGTGCAGCCAGGTGGCTCTCTGCGTTTGTCCT | PGKEREWVAGMSSAGDRSSYEDSVKGRF |
| | | GTGCCGCTTCTGGCTTCCCAGTGAACCGCTATTCC | TISRDDARNTVYLQMNSLKPEDTAVYYCN |
| | | ATGCGCTGGTATCGCCAGGCTCCAGGCAAAGAGC | VNVGFEYWGQGTQVTVSSGSGSGSGAPR |
| | | GTGAGTGGGTAGCCGGTATGTCCAGCGCGGGTGA | NGTVHLYLTKPLYTSAPSLQHLCRLTINKC |
| | | TCGTAGCTCCTATGAAGACTCCGTGAAGGGCCGTT | TGAIWGLPLPTRLKDYLEEYKFQV (SEQ |
| | | TCACCATCAGCCGTGACGATGCCCGTAACACGGTG | ID NO: 10) |
| | | TATCTGCAAATGAACAGCTTGAAACCTGAAGATA | |
| | | CGGCCGTGTATTACTGTAATGTGAACGTGGGCTTC | |
| | | GAGTATTGGGGCCAAGGCACCCAGGTCACCGTgTC | |
| | | CAGCGGCAGTGGTAGTGGCtccggaGCTCCACGAAA | |
| | | CGGCACGGTCCACCTTTACTTGACGAAACCGCTTT | |
| | | ATACCTCCGCACCGTCCCTCCAGCACCTGTGTCGG | |
| | | CTGACAATCAATAAGTGTACAGGTGCGATATGGG | |
| | | GATTGCCGCTTCCGACCCGCCTTAAAGATTACCTC | |
| | | GAGGAATACAAATTTCAAGTC (SEQ ID NO: 9) | |
| pJL_017 | vhhGFP4- | ATGggaggccttGATTACAAGGACGACGACGACAAGgc | MGGLDYKDDDDKASDQVQLVESGGALV |
| | SPOP | tagcGATCAAGTCCAACTGGTGGAGTCTGGTGGCGC | QPGGSLRLSCAASGFPVNRYSMRWYRQA |
| | | TTTGGTGCAGCCAGGTGGCTCTCTGCGTTTGTCCT | PGKEREWVAGMSSAGDRSSYEDSVKGRF |
| | | GTGCCGCTTCTGGCTTCCCAGTGAACCGCTATTCC | TISRDDARNTVYLQMNSLKPEDTAVYYCN |
| | | ATGCGCTGGTATCGCCAGGCTCCAGGCAAAGAGC | VNVGFEYWGQGTQVTVSSGSGSGSGSVNI |
| | | GTGAGTGGGTAGCCGGTATGTCCAGCGCGGGTGA | SGQNTMNMVKVPECRLADELGGLWENSR |
| | | TCGTAGCTCCTATGAAGACTCCGTGAAGGGCCGTT | FTDCCLCVAGQEFQAHKAILAARSPVFSA |
| | | TCACCATCAGCCGTGACGATGCCCGTAACACGGTG | MFEHEMEESKKNRVEINDVEPEVFKEMM |
| | | TATCTGCAAATGAACAGCTTGAAACCTGAAGATA | CFIYTGKAPNLDKMADDLLAAADKYALE |
| | | CGGCCGTGTATTACTGTAATGTGAACGTGGGCTTC | RLKVMCEDALCSNLSVENAAEILILADLHS |
| | | GAGTATTGGGGCCAAGGCACCCAGGTCACCGTgTC | ADQLKTQAVDFINYHASDVLETSGWKSM |
| | | CAGCGGCAGTGGTAGTGGCtccggaTCCGTAAATATA | VVSHPHLVAEAYRSLASAQCPFLGPPRKR |
| | | TCTGGGCAAAACACGATGAATATGGTAAAGGTCC | LKQS (SEQ ID NO: 12) |
| | | CCGAGTGCAGATTGGCCGATGAGCTGGGGGGATT | |
| | | GTGGGAGAACAGCCGGTTCACCGACTGTTGTCTCT | |
| | | GTGTAGCCGGCCAGGAGTTCCAAGCTCATAAGGC | |
| | | GATTCTTGCTGCCCGATCTCCAGTATTCAGCGCGA | |
| | | TGTTCGAACATGAGATGGAAGAGTCTAAGAAAAA | |
| | | CAGGGTCGAGATTAACGATGTAGAGCCCGAAGTC | |
| | | TTTAAGGAGATGATGTGTTTCATATACACGGGCAA | |
| | | GGCGCCTAATCTGGATAAGATGGCGGATGACTTG | |
| | | CTGGCAGCGGCGGATAAGTACGCCTTGGAACGCC | |
| | | TTAAGGTGATGTGTGAGGACGCTTTGTGCTCCAAT | |
| | | CTGAGCGTGGAGAACGCAGCAGAAATACTCATTC | |
| | | TGGCAGACTTGCATTCTGCGGATCAGTTGAAGACT | |
| | | CAGGCCGTAGATTTCATAAACTATCACGCGTCAGA | |
| | | TGTTCTCGAGACATCTGGCTGGAAATCCATGGTGG | |
| | | TGTCTCATCCTCATTTGGTTGCGGAGGCGTATCGC | |
| | | TCCCTGGCCTCTGCCCAATGTCCCTTCCTTGGGCC | |
| | | GCCTCGGAAAAGGCTGAAACAATCC (SEQ ID NO: 11) | |
| pJL_018 | vhhGFP4- | ATGggaggccttGATTACAAGGACGACGACGACAAGgc | MGGLDYKDDDDKASDQVQLVESGGALV |
| | VHL | tagcGATCAAGTCCAACTGGTGGAGTCTGGTGGCGC | QPGGSLRLSCAASGFPVNRYSMRWYRQA |
| | | TTTGGTGCAGCCAGGTGGCTCTCTGCGTTTGTCCT | PGKEREWVAGMSSAGDRSSYEDSVKGRF |
| | | GTGCCGCTTCTGGCTTCCCAGTGAACCGCTATTCC | TISRDDARNTVYLQMNSLKPEDTAVYYCN |
| | | ATGCGCTGGTATCGCCAGGCTCCAGGCAAAGAGC | VNVGFEYWGQGTQVTVSSGSGSGSGTLP |
| | | GTGAGTGGGTAGCCGGTATGTCCAGCGCGGGTGA | VYTLKERCLQVVRSLVKPENYRRLDIVRS |
| | | TCGTAGCTCCTATGAAGACTCCGTGAAGGGCCGTT | LYEDLEDHPNVQKDLERLTQERIAHQRMG |
| | | TCACCATCAGCCGTGACGATGCCCGTAACACGGTG | D (SEQ ID NO: 14) |
| | | TATCTGCAAATGAACAGCTTGAAACCTGAAGATA | |
| | | CGGCCGTGTATTACTGTAATGTGAACGTGGGCTTC | |
| | | GAGTATTGGGGCCAAGGCACCCAGGTCACCGTgTC | |
| | | CAGCGGCAGTGGTAGTGGCtccggaACGCTCCCGGTC | |
| | | TACACGCTCAAGGAGCGATGTCTGCAAGTGGTAC | |
| | | GAAGCTTGGTCAAGCCGGAAAACTATCGCCGGCT | |
| | | GGATATAGTTCGATCCCTTTATGAAGACCTTGAAG | |
| | | ACCACCCTAATGTTCAAAAGGATCTTGAGCGATTG | |
| | | ACTCAGGAAAGAATAGCTCACCAACGCATGGGGG | |
| | | AT (SEQ ID NO: 13) | |
| pJL_019 | vhhGFP4- | ATGggaggccttGATTACAAGGACGACGACGACAAGgc | MGGLDYKDDDDKASDQVQLVESGGALV |
| | VPR | tagcGATCAAGTCCAACTGGTGGAGTCTGGTGGCGC | QPGGSLRLSCAASGFPVNRYSMRWYRQA |
| | | TTTGGTGCAGCCAGGTGGCTCTCTGCGTTTGTCCT | PGKEREWVAGMSSAGDRSSYEDSVKGRF |
| | | GTGCCGCTTCTGGCTTCCCAGTGAACCGCTATTCC | TISRDDARNTVYLQMNSLKPEDTAVYYCN |
| | | ATGCGCTGGTATCGCCAGGCTCCAGGCAAAGAGC | VNVGFEYWGQGTQVTVSSGSGSGSGEQA |
| | | GTGAGTGGGTAGCCGGTATGTCCAGCGCGGGTGA | PEDQGPQREPHNEWTLELLEELKNEAVRH |
| | | TCGTAGCTCCTATGAAGACTCCGTGAAGGGCCGTT | FPRIWLHGLGQHIYETYGDTWAGVEAIIRI |
| | | TCACCATCAGCCGTGACGATGCCCGTAACACGGTG | LQQLLFIHFRIGCRHSRIGVTQQRRARNGA |
| | | TATCTGCAAATGAACAGCTTGAAACCTGAAGATA | SRS (SEQ ID NO: 16) |
| | | CGGCCGTGTATTACTGTAATGTGAACGTGGGCTTC | |
| | | GAGTATTGGGGCCAAGGCACCCAGGTCACCGTgTC | |
| | | CAGCGGCAGTGGTAGTGGCtccggaGAGCAAGCACC | |
| | | GGAAGATCAGGGACCACAGCGCGAACCACACAAT | |
| | | GAATGGACATTGGAATTGCTTGAGGAGCTTAAAA | |
| | | ATGAAGCTGTTAGACACTTCCCAAGAATCTGGCTG | |
| | | CACGGCTTGGGTCAGCACATATATGAAACGTATG | |
| | | GAGATACATGGGCAGGTGTCGAGGCTATCATCAG | |
| | | GATCCTTCAGCAGCTCCTCTTCATTCATTTCAGAAT | |
| | | AGGGTGTCGACATAGCAGAATAGGGGTTACCCAG | |
| | | CAACGCAGGGCGCGGAATGGTGCCAGTAGGAGT | |
| | | (SEQ ID NO: 15) | |
| pJL_020 | vhhGFP4- | ATGggaggccttGATTACAAGGACGACGACGACAAGgc | MGGLDYKDDDDKASDQVQLVESGGALV |
| | VPX | tagcGATCAAGTCCAACTGGTGGAGTCTGGTGGCGC | QPGGSLRLSCAASGFPVNRYSMRWYRQA |
| | | TTTGGTGCAGCCAGGTGGCTCTCTGCGTTTGTCCT | PGKEREWVAGMSSAGDRSSYEDSVKGRF |
| | | GTGCCGCTTCTGGCTTCCCAGTGAACCGCTATTCC | TISRDDARNTVYLQMNSLKPEDTAVYYCN |
| | | ATGCGCTGGTATCGCCAGGCTCCAGGCAAAGAGC | VNVGFEYWGQGTQVTVSSGSGSGSGADP |
| | | GTGAGTGGGTAGCCGGTATGTCCAGCGCGGGTGA | RERVPPGNSGEETIGEAFEWLDRTIEALNR |
| | | TCGTAGCTCCTATGAAGACTCCGTGAAGGGCCGTT | EAVNHLPRELIFQVWQRSWAYWHDEQG |
| | | TCACCATCAGCCGTGACGATGCCCGTAACACGGTG | MSTSYTKYRYLCIMQKAVYIHFKKGCTCL |
| | | TATCTGCAAATGAACAGCTTGAAACCTGAAGATA | GRGHGPGGWRPGPPPPPPPGLV (SEQ ID |
| | | CGGCCGTGTATTACTGTAATGTGAACGTGGGCTTC | NO: 18) |
| | | GAGTATTGGGGCCAAGGCACCCAGGTCACCGTgTC | |
| | | CAGCGGCAGTGGTAGTGGCtccggaGCAGACCCGAG | |
| | | GGAAAGAGTTCCACCAGGTAACAGTGGCGAAGAA | |
| | | ACGATCGGCGAAGCGTTCGAGTGGCTTGATCGGA | |
| | | CTATTGAGGCTCTTAATCGGGAGGCTGTGAACCAC | |
| | | CTGCCCCGGGAACTTATTTTCCAGGTCTGGCAGAG | |
| | | AAGTTGGGCCTACTGGCACGATGAGCAAGGCATG | |
| | | TCCACATCCTATACAAAGTATCGGTATCTTTGTAT | |
| | | AATGCAGAAGGCTGTATATATCCACTTTAAGAAG | |
| | | GGATGTACTTGTCTGGGTAGAGGTCATGGACCAG | |
| | | GAGGGTGGAGACCAGGCCCTCCACCTCCTCCACCT | |
| | | CCAGGTCTTGTG (SEQ ID NO: 17) | |
| pJL_021 | vhhGFP4- | ATGggaggccttGATTACAAGGACGACGACGACAAGgc | MGGLDYKDDDDKASDQVQLVESGGALV |
| | WHVX | tagcGATCAAGTCCAACTGGTGGAGTCTGGTGGCGC | QPGGSLRLSCAASGFPVNRYSMRWYRQA |
| | | TTTGGTGCAGCCAGGTGGCTCTCTGCGTTTGTCCT | PGKEREWVAGMSSAGDRSSYEDSVKGRF |
| | | GTGCCGCTTCTGGCTTCCCAGTGAACCGCTATTCC | TISRDDARNTVYLQMNSLKPEDTAVYYCN |
| | | ATGCGCTGGTATCGCCAGGCTCCAGGCAAAGAGC | VNVGFEYWGQGTQVTVSSGSGSGSGGPC |
| | | GTGAGTGGGTAGCCGGTATGTCCAGCGCGGGTGA | CLDVTCAELRTMDSTVNFVSWHANRQLG |
| | | TCGTAGCTCCTATGAAGACTCCGTGAAGGGCCGTT | MPS (SEQ ID NO: 20) |
| | | TCACCATCAGCCGTGACGATGCCCGTAACACGGTG | |
| | | TATCTGCAAATGAACAGCTTGAAACCTGAAGATA | |
| | | CGGCCGTGTATTACTGTAATGTGAACGTGGGCTTC | |
| | | GAGTATTGGGGCCAAGGCACCCAGGTCACCGTgTC | |
| | | CAGCGGCAGTGGTAGTGGCtccggaGGACCGTGTTGC | |
| | | TTGGacGTAACGTGTGCAGAATTGCGCACAATGGA | |
| | | TTCAACAGTGAACTTCGTgTCATGGCATGCCAATC | |
| | | GGCAATTGGGGATGCCAAGC (SEQ ID NO: 19) | |
| pJL_022 | 913Ik0- | ATGggaggccttGATTACAAGGACGACGACGACAAGgc | MGGLDYKDDDDKASFNVLMVHRRSHTG |
| | vhhGFP4 | tagcTTCAATGTCTTAATGGTTCATcggCGAAGCCATA | ERPLQCEICGFTCRQRGNLLRHIRLHTGER |
| | | CTGGTGAACGCCCATTGCAGTGCGAAATATGCGG | PFRCHLCNYACQRRDALSGSGSGSGDQVQ |
| | | CTTTACCTGCCGCCAGcgcGGTAACCTCCTCCGCCA | LVESGGALVQPGGSLRLSCAASGFPVNRY |
| | | CATTcgtCTGCACACAGGGGAAcggCCTTTTcggTGTC | SMRWYRQAPGKEREWVAGMSSAGDRSS |
| | | ACCTCTGCAACTATGCATGCCAAAGAAGAGATGC | YEDSVKGRFTISRDDARNTVYLQMNSLKP |
| | | GCTCAGCGGGTCAGGTAGTGGCtccggaGATCAAGTC | EDTAVYYCNVNVGFEYWGQGTQVTVSS |
| | | CAACTGGTGGAGTCTGGTGGCGCTTTGGTGCAGCC | (SEQ ID NO: 22) |
| | | AGGTGGCTCTCTGCGTTTGTCCTGTGCCGCTTCTG | |
| | | GCTTCCCAGTGAACCGCTATTCCATGCGCTGGTAT | |
| | | CGCCAGGCTCCAGGCAAAGAGCGTGAGTGGGTAG | |
| | | CCGGTATGTCCAGCGCGGGTGATCGTAGCTCCTAT | |
| | | GAAGACTCCGTGAAGGGCCGTTTCACCATCAGCC | |
| | | GTGACGATGCCCGTAACACGGTGTATCTGCAAATG | |
| | | AACAGCTTGAAACCTGAAGATACGGCCGTGTATT | |
| | | ACTGTAATGTGAACGTGGGCTTCGAGTATTGGGGC | |
| | | CAAGGCACCCAGGTCACCGTgTCCAGC (SEQ ID NO: 21) | |
| pJL_023 | bTrCP2- | ATGggaggccttGATTACAAGGACGACGACGACAAGgc | MGGLDYKDDDDKASDPAEAVLQEKALKF |
| | vhhGFP4 | tagcGACCCTGCTGAAGCCGTGCTGCAGGAGAAGGC | MNSSEREDCNNGEPPRKIIPEKNSLRQTYN |
| | | ACTCAAGTTTATGAATAGCTCAGAAAGGGAAGAC | SCARLCLNQETVCLASTAMKTENCVAKT |
| | | TGTAATAACGGAGAGCCTCCGAGGAAAATTATAC | KLANGTSSMIVPKQRKLSASYEKEKELCV |
| | | CCGAAAAGAACAGTCTGCGGCAAACTTACAATAG | KYFEQWSESDQVEFVEHLISQMCHYQHG |
| | | TTGTGCCAGACTTTGTTTGAACCAGGAAACCGTAT | HINSYLKPMLQRDFITALPARGLDHIAENI |
| | | GTTTGGCGTCTACCGCAATGAAGACAGAGAATTG | LSYLDAKSLCAAELVCKEWYRVTSDGML |
| | | CGTGGCGAAGACCAAACTCGCTAACGGAACATCA | WKKLIERMVRTDSLWRGLAERRGWGQY |
| | | AGCATGATAGTCCCCAAACAGAGAAAGCTTAGTG | LFKNKPPDGNAPPNSFYRALYPKIIQDIETI |
| | | CGTCTTACGAAAAGGAGAAGGAGCTGTGTGTCAA | ESNWRCGRHSLQRIHCRSGSGSGSGDQVQ |
| | | GTATTTCGAGCAGTGGAGTGAGAGCGACCAGGTA | LVESGGALVQPGGSLRLSCAASGFPVNRY |
| | | GAATTCGTGGAGCACCTCATCAGCCAGATGTGCCA | SMRWYRQAPGKEREWVAGMSSAGDRSS |
| | | TTATCAGCATGGCCACATAAACAGCTACTTGAAGC | YEDSVKGRFTISRDDARNTVYLQMNSLKP |
| | | CCATGCTTCAGCGAGACTTTATTACTGCATTGCCT | EDTAVYYCNVNVGFEYWGQGTQVTVSS |
| | | GCTAGAGGTCTGGATCACATTGCGGAAAATATCTT | (SEQ ID NO: 24) |
| | | GTCCTATTTGGATGCTAAGAGCCTTTGCGCGGCCG | |
| | | AACTGGTATGCAAAGAATGGTATAGGGTGACGTC | |
| | | TGACGGAATGTTGTGGAAGAAACTCATTGAGAGA | |
| | | ATGGTGCGCACGGACTCTCTCTGGAGAGGGCTGG | |
| | | CGGAGCGCAGAGGTTGGGGACAGTATTTGTTCAA | |
| | | GAATAAACCGCCTGACGGGAACGCTCCACCGAAC | |
| | | AGTTTTTATCGCGCTCTGTATCCAAAAATAATACA | |
| | | AGATATCGAAACAATTGAGTCTAACTGGCGATGC | |
| | | GGCAGGCACAGTCTGCAGCGCATCCATTGCCGAA | |
| | | GCGGGTCAGGTAGTGGCtccggaGATCAAGTCCAACT | |
| | | GGTGGAGTCTGGTGGCGCTTTGGTGCAGCCAGGTG | |
| | | GCTCTCTGCGTTTGTCCTGTGCCGCTTCTGGCTTCC | |
| | | CAGTGAACCGCTATTCCATGCGCTGGTATCGCCAG | |
| | | GCTCCAGGCAAAGAGCGTGAGTGGGTAGCCGGTA | |
| | | TGTCCAGCGCGGGTGATCGTAGCTCCTATGAAGAC | |
| | | TCCGTGAAGGGCCGTTTCACCATCAGCCGTGACGA | |
| | | TGCCCGTAACACGGTGTATCTGCAAATGAACAGCT | |
| | | TGAAACCTGAAGATACGGCCGTGTATTACTGTAAT | |
| | | GTGAACGTGGGCTTCGAGTATTGGGGCCAAGGCA | |
| | | CCCAGGTCACCGTgTCCAGC (SEQ ID NO: 23) | |
| pJL_024 | FBW7- | ATGggaggccttGATTACAAGGACGACGACGACAAGgc | MGGLDYKDDDDKASCVPRSGLILSCICLY |
| | vhhGFP4 | tagcTGCGTACCGAGATCTGGACTGATATTGAGTTG | CGVLLPVLLPNLPFLTCLSMSTLESVTYLP |
| | | TATCTGCCTTTACTGTGGCGTACTGCTCCCCGTTTT | EKGLYCQRLPSSRTHGGTESLKGKNTENM |
| | | GTTGCCGAATCTCCCGTTTCTGACATGTCTCTCTAT | GFYGTLKMIFYKMKRKLDHGSEVRSFSLG |
| | | GTCAACCTTGGAGAGCGTGACCTACCTGCCAGAG | KKPCKVSEYTSTTGLVPCSATPTTFGDLRA |
| | | AAAGGTCTGTACTGTCAGCGCTTGCCCTCATCCAG | ANGQGQQRRRITSVQPPTGLQEWLKMFQS |
| | | GACACATGGCGGAACAGAATCTCTGAAAGGGAAG | WSGPEKLLALDELIDSCEPTQVKHMMQVI |
| | | AATACGGAGAATATGGGATTTTACGGAACGTTGA | EPQFQRDFISLLPKELALYVLSFLEPKDLLQ |
| | | AAATGATTTTCTACAAGATGAAACGGAAACTCGA | AAQTCRYWRILAEDNLLWREKCKEEGIDE |
| | | TCACGGAAGCGAAGTACGGTCATTTTCTCTTGGGA | PLHIKRRKVIKPGFIHSPWKSAYIRQHRIDT |
| | | AGAAGCCTTGTAAAGTGTCCGAGTATACATCAAC | NWRRGELKSPSGSGSGSGDQVQLVESGG |
| | | GACGGGGCTGGTCCCTTGTAGTGCCACACCTACCA | ALVQPGGSLRLSCAASGFPVNRYSMRWY |
| | | CCTTCGGTGATCTTAGAGCAGCTAACGGTCAAGGC | RQAPGKEREWVAGMSSAGDRSSYEDSVK |
| | | CAACAAAGGCGGCGCATCACGTCTGTTCAACCAC | GRFTISRDDARNTVYLQMNSLKPEDTAVY |
| | | CTACCGGTCTTCAGGAATGGCTTAAGATGTTCCAG | YCNVNVGFEYWGQGTQVTVSS (SEQ ID |
| | | TCTTGGTCTGGACCCGAGAAATTGCTTGCCCTCGA | NO: 26) |
| | | CGAGCTGATTGACAGTTGTGAACCAACACAAGTT | |
| | | AAACACATGATGCAAGTTATCGAGCCTCAGTTTCA | |
| | | ACGAGACTTTATTTCTTTGCTTCCGAAGGAATTGG | |
| | | CTCTGTACGTGCTTTCATTTCTTGAACCCAAGGAC | |
| | | CTCCTGCAAGCCGCCCAAACGTGCCGCTATTGGCG | |
| | | CATCCTTGCGGAAGATAATTTGCTCTGGAGGGAAA | |
| | | AGTGTAAGGAGGAAGGAATTGATGAACCACTGCA | |
| | | TATTAAACGGCGCAAGGTCATTAAACCAGGATTTA | |
| | | TACATTCACCTTGGAAGTCTGCATACATACGCCAG | |
| | | CATCGCATCGACACTAACTGGCGACGAGGCGAGT | |
| | | TGAAATCCCCGAGCGGGTCAGGTAGTGGCtccggaG | |
| | | ATCAAGTCCAACTGGTGGAGTCTGGTGGCGCTTTG | |
| | | GTGCAGCCAGGTGGCTCTCTGCGTTTGTCCTGTGC | |
| | | CGCTTCTGGCTTCCCAGTGAACCGCTATTCCATGC | |
| | | GCTGGTATCGCCAGGCTCCAGGCAAAGAGCGTGA | |
| | | GTGGGTAGCCGGTATGTCCAGCGCGGGTGATCGT | |
| | | AGCTCCTATGAAGACTCCGTGAAGGGCCGTTTCAC | |
| | | CATCAGCCGTGACGATGCCCGTAACACGGTGTATC | |
| | | TGCAAATGAACAGCTTGAAACCTGAAGATACGGC | |
| | | CGTGTATTACTGTAATGTGAACGTGGGCTTCGAGT | |
| | | ATTGGGGCCAAGGCACCCAGGTCACCGTgTCCAGC | |
| | | (SEQ ID NO: 25) | |
| pJL_025 | HBVX- | ATGAACGCGCATCAAATATTGCCTAAGGTCCTGCA | MNAHQILPKVLHKRTLGLSGSGSGSGDQV |
| | vhhGFP4 | TAAACGAACCTTGGGTCTTAGCGGGTCAGGTAGTG | QLVESGGALVQPGGSLRLSCAASGFPVNR |
| | | GCtccggaGATCAAGTCCAACTGGTGGAGTCTGGTGG | YSMRWYRQAPGKEREWVAGMSSAGDRS |
| | | CGCTTTGGTGCAGCCAGGTGGCTCTCTGCGTTTGT | SYEDSVKGRFTISRDDARNTVYLQMNSLK |
| | | CCTGTGCCGCTTCTGGCTTCCCAGTGAACCGCTAT | PEDTAVYYCNVNVGFEYWGQGTQVTVSS |
| | | TCCATGCGCTGGTATCGCCAGGCTCCAGGCAAAG | (SEQ ID NO: 28) |
| | | AGCGTGAGTGGGTAGCCGGTATGTCCAGCGCGGG | |
| | | TGATCGTAGCTCCTATGAAGACTCCGTGAAGGGCC | |
| | | GTTTCACCATCAGCCGTGACGATGCCCGTAACACG | |
| | | GTGTATCTGCAAATGAACAGCTTGAAACCTGAAG | |
| | | ATACGGCCGTGTATTACTGTAATGTGAACGTGGGC | |
| | | TTCGAGTATTGGGGCCAAGGCACCCAGGTCACCGT | |
| | | gTCCAGC (SEQ ID NO: 27) | |
| pJL_026 | SKP2- | ATGCATCGAAAGCATCTTCAGGAGATACCTGACTT | MHRKHLQEIPDLSSNVATSFTWGWDSSKT |
| | vhhGFP4 | GTCATCCAATGTAGCCACGTCCTTCACCTGGGGCT | SELLSGMGVSALEKEEPDSENIPQELLSNL |
| | | GGGACAGCAGCAAGACCTCCGAACTTCTGTCCGG | GHPESPPRKRLKSKGSDKDFVIVRRPKLNR |
| | | AATGGGGGTCTCTGCTCTGGAGAAGGAGGAGCCT | ENFPGVSWDSLPDELLLGIFSCLCLPELLK |
| | | GACAGCGAAAATATCCCCCAGGAATTGCTCTCAA | VSGVCKRWYRLASDESLWQTLDLTGKNL |
| | | ACCTTGGTCATCCCGAGTCACCTCCTCGGAAGCGC | GSAGSAAGSGEFVQLVESGGALVQPGGSL |
| | | TTGAAGTCTAAGGGCTCCGACAAGGACTTCGTAAT | RLSCAASGFPVNRYSMRWYRQAPGKERE |
| | | AGTAAGGAGGCCCAAACTTAACCGAGAGAACTTT | WVAGMSSAGDRSSYEDSVKGRFTISRDDA |
| | | CCTGGGGTCAGTTGGGATAGTCTGCCGGACGAGCT | RNTVYLQMNSLKPEDTAVYYCNVNVGFE |
| | | GCTCCTCGGGATATTCTCCTGCTTGTGTCTGCCGG | YWGQGTQVTVSS (SEQ ID NO: 30) |
| | | AACTTCTTAAGGTTTCTGGTGTCTGTAAGCGCTGG | |
| | | TATCGCCTCGCTTCTGACGAGAGCCTTTGGCAGAC | |
| | | GTTGGACCTTACCGGGAAAAACCTTGGTTCCGCAG | |
| | | GGTCAGCAGCCGGATCTGGAGAGTTTgtccaactggtgga | |
| | | gtctggtggcgctttggtgcagccaggtggctctctgcgt | |
| | | ttgtcctgtgccgcttctggcttcccagtgaaccgctat | |
| | | tccatgcgctggtatogccaggctccaggcaaaga | |
| | | gcgtgagtgggtagccggtatgtccagcgcgggtga | |
| | | tcgtagctcctatgaagactccgtgaagggccgtt | |
| | | tcaccatcagccgtgacgatgcccgtaacacggtgtatctg | |
| | | caaatgaacagcttgaaacctgaagatacggccg | |
| | | tgtattactgtaatgtgaacgtgggcttcgagtattgg | |
| | | ggccaaggcacccaggtcaccgtctccagc (SEQ ID | |
| | | NO: 29) | |
| pJL_027 | VPR- | ATGggaggccttGATTACAAGGACGACGACGACAAGgc | MGGLDYKDDDDKASEQAPEDQGPQREPH |
| | vhhGFP4 | tagcGAGCAAGCACCGGAAGATCAGGGACCACAGC | NEWTLELLEELKNEAVRHFPRIWLHGLGQ |
| | | GCGAACCACACAATGAATGGACATTGGAATTGCT | HIYETYGDTWAGVEAIIRILQQLLFIHFRIG |
| | | TGAGGAGCTTAAAAATGAAGCTGTTAGACACTTCC | CRHSRIGVTQQRRARNGASRSGSGSGSGD |
| | | CAAGAATCTGGCTGCACGGCTTGGGTCAGCACAT | QVQLVESGGALVQPGGSLRLSCAASGFPV |
| | | ATATGAAACGTATGGAGATACATGGGCAGGTGTC | NRYSMRWYRQAPGKEREWVAGMSSAGD |
| | | GAGGCTATCATCAGGATCCTTCAGCAGCTCCTCTT | RSSYEDSVKGRFTISRDDARNTVYLQMNS |
| | | CATTCATTTCAGAATAGGGTGTCGACATAGCAGAA | LKPEDTAVYYCNVNVGFEYWGQGTQVTV |
| | | TAGGGGTTACCCAGCAACGCAGGGCGCGGAATGG | SS (SEQ ID NO: 32) |
| | | TGCCAGTAGGAGCGGGTCAGGTAGTGGCtccggaGA | |
| | | TCAAGTCCAACTGGTGGAGTCTGGTGGCGCTTTGG | |
| | | TGCAGCCAGGTGGCTCTCTGCGTTTGTCCTGTGCC | |
| | | GCTTCTGGCTTCCCAGTGAACCGCTATTCCATGCG | |
| | | CTGGTATCGCCAGGCTCCAGGCAAAGAGCGTGAG | |
| | | TGGGTAGCCGGTATGTCCAGCGCGGGTGATCGTA | |
| | | GCTCCTATGAAGACTCCGTGAAGGGCCGTTTCACC | |
| | | ATCAGCCGTGACGATGCCCGTAACACGGTGTATCT | |
| | | GCAAATGAACAGCTTGAAACCTGAAGATACGGCC | |
| | | GTGTATTACTGTAATGTGAACGTGGGCTTCGAGTA | |
| | | TTGGGGCCAAGGCACCCAGGTCACCGTgTCCAGC | |
| | | (SEQ ID NO: 31) | |
| pJL_028 | VPX- | ATGGCAGACCCGAGGGAAAGAGTTCCACCAGGTA | MADPRERVPPGNSGEETIGEAFEWLDRTIE |
| | vhhGFP4 | ACAGTGGCGAAGAAACGATCGGCGAAGCGTTCGA | ALNREAVNHLPRELIFQVWQRSWAYWHD |
| | | GTGGCTTGATCGGACTATTGAGGCTCTTAATCGGG | EQGMSTSYTKYRYLCIMQKAVYIHFKKGC |
| | | AGGCTGTGAACCACCTGCCCCGGGAACTTATTTTC | TCLGRGHGPGGWRPGPPPPPPPGLVGSAG |
| | | CAGGTCTGGCAGAGAAGTTGGGCCTACTGGCACG | SAAGSGEFVQLVESGGALVQPGGSLRLSC |
| | | ATGAGCAAGGCATGTCCACATCCTATACAAAGTAT | AASGFPVNRYSMRWYRQAPGKEREWVA |
| | | CGGTATCTTTGTATAATGCAGAAGGCTGTATATAT | GMSSAGDRSSYEDSVKGRFTISRDDARNT |
| | | CCACTTTAAGAAGGGATGTACTTGTCTGGGTAGAG | VYLQMNSLKPEDTAVYYCNVNVGFEYW |
| | | GTCATGGACCAGGAGGGTGGAGACCAGGCCCTCC | GQGTQVTVSS (SEQ ID NO: 34) |
| | | ACCTCCTCCACCTCCAGGTCTTGTGGGTTCCGCAG | |
| | | GGTCAGCAGCCGGATCTGGAGAGTTTgtccaactggtgga | |
| | | gtctggtggcgctttggtgcagccaggtggctctc | |
| | | tgcgtttgtcctgtgccgcttctggcttcccagtgaac | |
| | | cgctattccatgcgctggtatcgccaggctccaggcaaaga | |
| | | gcgtgagtgggtagccggtatgtccagcgcgggtga | |
| | | tcgtagctcctatgaagactccgtgaagggccgtttcac | |
| | | catcagccgtgacgatgcccgtaacacggtgtatctg | |
| | | caaatgaacagcttgaaacctgaagatacggccgt | |
| | | gtattactgtaatgtgaacgtgggcttcgagtat | |
| | | tggggccaaggcacccaggtcaccgtctccagc (SEQ ID | |
| | | NO: 33) | |
| pJL_029 | WHVX- | ATGGGACCGTGTTGCTTGGacGTAACGTGTGCAGA | MGPCCLDVTCAELRTMDSTVNFVSWHAN |
| | vhhGFP4 | ATTGCGCACAATGGATTCAACAGTGAACTTCGTCT | RQLGMPSGSAGSAAGSGEFVQLVESGGAL |
| | | CATGGCATGCCAATCGGCAATTGGGGATGCCAAG | VQPGGSLRLSCAASGFPVNRYSMRWYRQ |
| | | CGGTTCCGCAGGGTCAGCAGCCGGATCTGGAGAG | APGKEREWVAGMSSAGDRSSYEDSVKGR |
| | | TTTgtccaactggtggagtctggtggcgctttggtgc | FTISRDDARNTVYLQMNSLKPEDTAVYYC |
| | | agccaggtggctctctgcgtttgtcctgtgccgct | NVNVGFEYWGQGTQVTVSS (SEQ ID NO: |
| | | tctggcttcccagtgaaccgctattccatgcgctggt | 36) |
| | | atcgccaggctccaggcaaagagcgtgagtgggta | |
| | | gccggtatgtccagcgcgggtgatcgtagctc | |
| | | ctatgaagactccgtgaagggccgtttcaccatcagccg | |
| | | tgacgatgcccgtaacacggtgtatctgcaaatgaaca | |
| | | gcttgaaacctgaagatacggccgtgtattactgta | |
| | | atgtgaacgtgggcttcgagtattggggccaaggcacccaggtcacc | |
| | | gtctccagc (SEQ ID NO: 35) | |
| pJL_039 | PD1 | ATGCAAATACCTCAAGCGCCCTGGCCCGTAGTGTG | MQIPQAPWPVVWAVLQLGWRPGWFLDSP |
| | | GGCAGTCCTCCAACTTGGCTGGAGGCCAGGATGG | DRPWNPPTFSPALLVVTEGDNATFTCSFSN |
| | | TTCTTGGACAGCCCTGACCGCCCTTGGAATCCCCC | TSESFVLNWYRMSPSNQTDKLAAFPEDRS |
| | | GACTTTTTCACCAGCGCTGCTCGTTGTCACAGAAG | QPGQDCRFRVTQLPNGRDFHMSVVRARR |
| | | GAGACAATGCTACGTTTACTTGTTCTTTCTCTAAC | NDSGTYLCGAISLAPKAQIKESLRAELRVT |
| | | ACTTCAGAATCATTTGTTCTCAATTGGTATAGGAT | ERRAEVPTAHPSPSPRPAGQFQTLVVGVV |
| | | GAGTCCCTCTAATCAGACAGATAAACTGGCGGCA | GGLLGSLVLLVWVLAVICSRAARGTIGAR |
| | | TTTCCAGAGGATCGCAGTCAGCCGGGGCAGGACT | RTGQPLKEDPSAVPVFSVDYGELDFQWRE |
| | | GCCGATTTAGGGTTACCCAGCTCCCCAATGGGCGA | KTPEPPVPCVPEQTEYATIVFPSGMGTSSP |
| | | GACTTTCACATGTCTGTGGTCCGGGCCCGCAGAAA | ARRGSADGPRSAQPLRPEDGHCSWPL |
| | | TGACTCTGGTACTTATCTCTGTGGGGCTATAAGCT | (SEQ ID NO: 38) |
| | | TGGCGCCGAAAGCACAAATAAAAGAAAGCTTGCG | |
| | | CGCGGAACTGCGCGTTACAGAAAGAAGGGCAGAG | |
| | | GTGCCCACAGCTCATCCGAGCCCCTCTCCGCGGCC | |
| | | AGCGGGACAGTTTCAAACACTGGTCGTAGGAGTA | |
| | | GTAGGCGGTTTGCTGGGATCATTGGTACTTCTTGT | |
| | | ATGGGTGTTGGCCGTAATATGCAGCCGAGCTGCG | |
| | | AGAGGAACAATTGGCGCAAGAAGGACCGGGCAGC | |
| | | CACTGAAAGAGGACCCATCAGCCGTACCGGTGTTT | |
| | | TCAGTTGACTACGGAGAATTGGACTTCCAGTGGCG | |
| | | CGAGAAAACCCCAGAACCACCTGTTCCCTGTGTTC | |
| | | CTGAACAAACTGAGTACGCAACTATCGTGTTTCCA | |
| | | TCAGGCATGGGTACTTCTTCCCCGGCCCGAAGAGG | |
| | | TTCCGCAGACGGACCCAGGTCTGCACAGCCCCTTC | |
| | | GCCCGGAGGACGGTCATTGTTCCTGGCCACTG | |
| | | (SEQ ID NO: 37) | |
| pJL_040 | myr- | ATGGGTTGCATCAAGAGCAAGCGGAAAGATAACC | MGCIKSKRKDNLNDDGVDMKTGSGSGSG |
| | vhhGFP4- | TCAACGATGATGGGGTTGACATGAAAACAggcagtgg | SGGLDYKDDDDKASDQVQLVESGGALVQ |
| | SOCS2 | ctcgggctcggggtccggcggacttGATTACAAGGACGACGAC | PGGSLRLSCAASGFPVNRYSMRWYRQAP |
| | | GACAAGgctagcGATCAAGTCCAACTGGTGGAGTCT | GKEREWVAGMSSAGDRSSYEDSVKGRFTI |
| | | GGTGGCGCTTTGGTGCAGCCAGGTGGCTCTCTGCG | SRDDARNTVYLQMNSLKPEDTAVYYCNV |
| | | TTTGTCCTGTGCCGCTTCTGGCTTCCCAGTGAACC | NVGFEYWGQGTQVTVSSGSGSGSGAPRN |
| | | GCTATTCCATGCGCTGGTATCGCCAGGCTCCAGGC | GTVHLYLTKPLYTSAPSLQHLCRLTINKCT |
| | | AAAGAGCGTGAGTGGGTAGCCGGTATGTCCAGCG | GAIWGLPLPTRLKDYLEEYKFQV (SEQ ID |
| | | CGGGTGATCGTAGCTCCTATGAAGACTCCGTGAAG | NO: 40) |
| | | GGCCGTTTCACCATCAGCCGTGACGATGCCCGTAA | |
| | | CACGGTGTATCTGCAAATGAACAGCTTGAAACCTG | |
| | | AAGATACGGCCGTGTATTACTGTAATGTGAACGTG | |
| | | GGCTTCGAGTATTGGGGCCAAGGCACCCAGGTCA | |
| | | CCGTgTCCAGCGGCAGTGGTAGTGGCtccggaGCTCC | |
| | | ACGAAACGGCACGGTCCACCTTTACTTGACGAAA | |
| | | CCGCTTTATACCTCCGCACCGTCCCTCCAGCACCT | |
| | | GTGTCGGCTGACAATCAATAAGTGTACAGGTGCG | |
| | | ATATGGGGATTGCCGCTTCCGACCCGCCTTAAAGA | |
| | | TTACCTCGAGGAATACAAATTTCAAGTC (SEQ ID | |
| | | NO: 39) | |
| pJL_042 | myr- | ATGGGTTGCATCAAGAGCAAGCGGAAAGATAACC | MGCIKSKRKDNLNDDGVDMKTGSGSGSG |
| | vhhGFP4- | TCAACGATGATGGGGTTGACATGAAAACAggcagtgg | SGGLDYKDDDDKASDQVQLVESGGALVQ |
| | VPX | ctcgggctcggggtccggcggacttGATTACAAGGACGACGAC | PGGSLRLSCAASGFPVNRYSMRWYRQAP |
| | | GACAAGgctagcGATCAAGTCCAACTGGTGGAGTCT | GKEREWVAGMSSAGDRSSYEDSVKGRFTI |
| | | GGTGGCGCTTTGGTGCAGCCAGGTGGCTCTCTGCG | SRDDARNTVYLQMNSLKPEDTAVYYCNV |
| | | TTTGTCCTGTGCCGCTTCTGGCTTCCCAGTGAACC | NVGFEYWGQGTQVTVSSGSGSGSGADPR |
| | | GCTATTCCATGCGCTGGTATCGCCAGGCTCCAGGC | ERVPPGNSGEETIGEAFEWLDRTIEALNRE |
| | | AAAGAGCGTGAGTGGGTAGCCGGTATGTCCAGCG | AVNHLPRELIFQVWQRSWAYWHDEQGM |
| | | CGGGTGATCGTAGCTCCTATGAAGACTCCGTGAAG | STSYTKYRYLCIMQKAVYIHFKKGCTCLG |
| | | GGCCGTTTCACCATCAGCCGTGACGATGCCCGTAA | RGHGPGGWRPGPPPPPPPGLV (SEQ ID |
| | | CACGGTGTATCTGCAAATGAACAGCTTGAAACCTG | NO: 42) |
| | | AAGATACGGCCGTGTATTACTGTAATGTGAACGTG | |
| | | GGCTTCGAGTATTGGGGCCAAGGCACCCAGGTCA | |
| | | CCGTgTCCAGCGGCAGTGGTAGTGGCtccggaGCAGA | |
| | | CCCGAGGGAAAGAGTTCCACCAGGTAACAGTGGC | |
| | | GAAGAAACGATCGGCGAAGCGTTCGAGTGGCTTG | |
| | | ATCGGACTATTGAGGCTCTTAATCGGGAGGCTGTG | |
| | | AACCACCTGCCCCGGGAACTTATTTTCCAGGTCTG | |
| | | GCAGAGAAGTTGGGCCTACTGGCACGATGAGCAA | |
| | | GGCATGTCCACATCCTATACAAAGTATCGGTATCT | |
| | | TTGTATAATGCAGAAGGCTGTATATATCCACTTTA | |
| | | AGAAGGGATGTACTTGTCTGGGTAGAGGTCATGG | |
| | | ACCAGGAGGGTGGAGACCAGGCCCTCCACCTCCT | |
| | | CCACCTCCAGGTCTTGTG (SEQ ID NO: 41) | |
| pJL_044 | myr-913iK0- | ATGGGTTGCATCAAGAGCAAGCGGAAAGATAACC | MGCIKSKRKDNLNDDGVDMKTGSGSGSG |
| | vhhGFP4 | TCAACGATGATGGGGTTGACATGAAAACAggcagtgg | SGGLDYKDDDDKASFNVLMVHRRSHTGE |
| | | ctcgggctcggggtccggcggacttGATTACAAGGACGACGAC | RPLQCEICGFTCRQRGNLLRHIRLHTGERP |
| | | GACAAGgctagcTTCAATGTCTTAATGGTTCATcggCG | FRCHLCNYACQRRDALSGSGSGSGDQVQ |
| | | AAGCCATACTGGTGAACGCCCATTGCAGTGCGAA | LVESGGALVQPGGSLRLSCAASGFPVNRY |
| | | ATATGCGGCTTTACCTGCCGCCAGcgcGGTAACCTC | SMRWYRQAPGKEREWVAGMSSAGDRSS |
| | | CTCCGCCACATTcgtCTGCACACAGGGGAAcggCCTT | YEDSVKGRFTISRDDARNTVYLQMNSLKP |
| | | TTcggTGTCACCTCTGCAACTATGCATGCCAAAGAA | EDTAVYYCNVNVGFEYWGQGTQVTVSS |
| | | GAGATGCGCTCAGCGGGTCAGGTAGTGGCtccggaG | (SEQ ID NO: 44) |
| | | ATCAAGTCCAACTGGTGGAGTCTGGTGGCGCTTTG | |
| | | GTGCAGCCAGGTGGCTCTCTGCGTTTGTCCTGTGC | |
| | | CGCTTCTGGCTTCCCAGTGAACCGCTATTCCATGC | |
| | | GCTGGTATCGCCAGGCTCCAGGCAAAGAGCGTGA | |
| | | GTGGGTAGCCGGTATGTCCAGCGCGGGTGATCGT | |
| | | AGCTCCTATGAAGACTCCGTGAAGGGCCGTTTCAC | |
| | | CATCAGCCGTGACGATGCCCGTAACACGGTGTATC | |
| | | TGCAAATGAACAGCTTGAAACCTGAAGATACGGC | |
| | | CGTGTATTACTGTAATGTGAACGTGGGCTTCGAGT | |
| | | ATTGGGGCCAAGGCACCCAGGTCACCGTgTCCAGC | |
| | | (SEQ ID NO: 43) | |
| pJL_054 | TET2 | AAGGGCGGCGGCGGCTCCGGCGGCGGCGGCTCCG | KGGGGSGGGGSDFPSCRCVEQIIEKDEGPF |
| | catalytic | ACTTCCCTTCCTGCAGGTGCGTTGAACAAATAATT | YTHLGAGPNVAAIREIMEERFGQKGKAIRI |
| | | GAAAAAGACGAGGGTCCATTTTATACACATCTGG | ERVIYTGKEGKSSQGCPAIKWVVRRSSSEE |
| | | GAGCTGGACCAAACGTCGCGGCAATCCGCGAGAT | KLLCLVRERAGHTCEAAVIVILILVWEGIP |
| | | AATGGAAGAACGGTTCGGCCAAAAAGGGAAAGCA | LSLADKLYSELTETLRKYGTLNTNRRCALN |
| | | ATAAGAATCGAACGCGTTATTTACACTGGTAAGG | EERTCACQGLDPETCGASFSFGCSWSMYY |
| | | AGGGTAAGAGTAGCCAGGGGTGCCCAATAGCTAA | NGCKFARSKIPRKFKLLGDDPKEEEKLESH |
| | | ATGGGTCGTCAGAAGGTCCTCAAGTGAAGAGAAA | LQNLSTLMAPTYKKLAPDAYNNQIEYEHR |
| | | CTCCTCTGCCTTGTCCGAGAAAGGGCTGGCCACAC | APECRLGLKEGRPFSGVTACLDFCAHAHR |
| | | TTGTGAGGCAGCCGTCATCGTCATCTTGATTCTGG | DLHNMQNGSTLVCTLTREDNREFGGKPE |
| | | TCTGGGAGGGGATCCCCCTTAGTCTCGCAGACAAA | DEQLHVLPLYKVSDVDEFGSVEAQEEKKR |
| | | CTCTATTCCGAGCTCACCGAAACGCTTCGCAAATA | SQAIQVLSSFRRKVRMLAEPVKTCRQRKL |
| | | TGGGACTCTGACAAATAGGAGGTGCGCCCTGAAT | EAKKAAAEKLSSLENSSNKNEKEKSAPSR |
| | | GAAGAGAGAACATGCGCATGCCAAGGCTTGGATC | TKQTENASQAKQLAELLRLSGPVMQQSQ |
| | | CCGAGACCTGTGGTGCCAGCTTTTCCTTTGGATGT | QPQPLQKQPPQPQQQQRPQQQQPHHPQTE |
| | | AGCTGGAGTATGTACTACAACGGCTGTAAGTTTGC | SVNSYSASGSTNPYMRRPNPVSPYPNSSHT |
| | | AAGGTCCAAAATTCCTCGCAAATTTAAGTTGCTCG | SDIYGSTSPMNFYSTSSQAAGSYLNSSNPM |
| | | GCGACGACCCTAAGGAAGAAGAAAAGCTCGAGAG | NPYPGLLNQNTQYPSYQCNGNLSVDNCSP |
| | | TCATCTCCAAAACTTGAGTACTCTCATGGCCCCTA | YLGSYSPQSQPMDLYRYPSQDPLSKLSLPP |
| | | CATATAAGAAACTGGCTCCGGATGCATATAACAA | IHTLYQPRFGNSQSFTSKYLGYGNQNMQG |
| | | TCAGATAGAATATGAGCACAGGGCACCCGAGTGC | DGFSSCTIRPNVHHVGKLPPYPTHEMDGH |
| | | AGACTGGGATTGAAGGAAGGGAGGCCTTTTTCAG | FMGATSRLPPNLSNPNMDYKNGEHHSPSH |
| | | GTGTAACGGCTTGCCTCGACTTTTGTGCTCACGCT | IIHNYSAAPGMFNSSLHALHLQNKENDML |
| | | CACAGGGATCTGCACAACATGCAGAACGGCAGCA | SHTANGLSKMLPALNHDRTACVQGGLHK |
| | | CACTCGTTTGTACTCTGACCAGAGAAGACAATCGA | LSDANGQEKQPLALVQGVASGAEDNDEV |
| | | GAGTTCGGTGGGAAACCTGAGGACGAACAACTTC | WSDSEQSFLDPDIGGVAVAPTHGSILIECA |
| | | ACGTCTTGCCACTGTACAAAGTCTCTGATGTTGAT | KRELHATTPLKNPNRNHPTRISLVFYQHKS |
| | | GAATTTGGGTCAGTTGAAGCCCAAGAGGAAAAAA | MNEPKHGLALWEAKMAEKAREKEEECEK |
| | | AACGATCAGGGGCGATCCAAGTACTGTCATCATTT | YGPDYVPQKSHGKKVKREPAEPHETSEPT |
| | | CGGAGAAAGGTAAGAATGCTTGCCGAGCCTGTCA | YLRFIKSLAERTMSVTTDSTVTTSPYAFTR |
| | | AAACGTGCCGCCAACGCAAACTGGAGGCCAAAAA | VTGPYNRYI* (SEQ ID NO: 46) |
| | | GGCAGCAGCAGAAAAGTTGAGTTCTCTTGAGAAC | |
| | | AGCTCAAACAAAAACGAGAAAGAAAAGAGCGCA | |
| | | CCGTCACGGACAAAGCAGACAGAAAACGCTTCAC | |
| | | AAGCGAAGCAGCTCGCAGAACTCCTTCGGTTGTCA | |
| | | GGCCCCGTCATGCAACAAAGTCAACAGCCCCAGC | |
| | | CGCTGCAGAAACAGCCACCCCAGCCCCAACAGCA | |
| | | ACAGAGACCGCAACAACAACAGCCCCATCACCCG | |
| | | CAGACAGAAAGCGTAAATAGCTATTCCGCGTCAG | |
| | | GGTCCACCAACCCTTATATGCGGAGGCCCAATCCT | |
| | | GTGAGTCCATATCCCAATTCCAGTCACACTTCCGA | |
| | | TATCTATGGGAGCACAAGCCCCATGAATTTTTACT | |
| | | CTACTTCATCTCAGGCCGCAGGTTCATATCTTAAT | |
| | | AGCTCCAATCCGATGAATCCCTATCCCGGCCTCCT | |
| | | TAATCAGAATACCCAGTACCCATCATACCAATGCA | |
| | | ACGGAAACCTGTCAGTTGATAACTGCTCCCCGTAC | |
| | | CTTGGAAGCTACTCCCCACAGTCCCAGCCCATGGA | |
| | | TCTGTACCGGTATCCAAGTCAGGACCCACTGTCTA | |
| | | AATTGAGTCTGCCGCCAATTCACACTCTTTATCAA | |
| | | CCGAGATTTGGAAACAGCCAGTCATTCACATCTAA | |
| | | ATACCTGGGTTATGGAAATCAGAATATGCAAGGA | |
| | | GATGGATTCTCCTCATGTACCATTCGACCAAACGT | |
| | | TCACCATGTTGGCAAGCTCCCACCTTACCCCACCC | |
| | | ATGAAATGGATGGTCATTTCATGGGAGCGACcTCT | |
| | | CGATTGCCGCCAAATCTGAGTAATCCTAACATGGA | |
| | | TTATAAAAATGGTGAACACCATAGCCCTTCCCATA | |
| | | TTATCCATAACTATTCTGCAGCCCCAGGGATGTTT | |
| | | AACTCTTCATTGCACGCTTTGCATTTGCAGAACAA | |
| | | GGAGAATGATATGCTGTCCCATACCGCAAACGGA | |
| | | CTCAGTAAAATGCTTCCAGCGCTTAATCATGACCG | |
| | | GACAGCGTGCGTCCAAGGTGGGCTTCACAAGCTTT | |
| | | CCGACGCCAACGGTCAGGAGAAGCAGCCTCTGGC | |
| | | TTTGGTCCAGGGGGTGGCGTCCGGAGCAGAAGAC | |
| | | AACGACGAAGTATGGAGCGATAGCGAGCAGAGCT | |
| | | TCCTGGATCCTGACATTGGGGGCGTGGCCGTGGCA | |
| | | CCGACCCATGGCTCTATCCTTATAGAGTGCGCGAA | |
| | | ACGAGAGCTCCACGCCACCACACCGCTTAAGAAT | |
| | | CCCAATCGCAATCATCCAACACGCATCAGTCTTGT | |
| | | ATTTTATCAACACAAGTCCATGAATGAGCCCAAGC | |
| | | ACGGTCTGGCCCTTTGGGAAGCTAAAATGGCTGA | |
| | | AAAAGCGAGGGAGAAGGAAGAAGAATGCGAAAA | |
| | | GTATGGGCCCGATTACGTTCCTCAAAAAAGTCATG | |
| | | GAAAAAAAGTCAAGCGCGAGCCAGCTGAACCTCA | |
| | | CGAGACTTCAGAACCTACGTACTTGAGATTCATTA | |
| | | AGTCTCTTGCTGAACGGACCATGTCTGTAACCACA | |
| | | GACTCAACGGTTACCACCTCTCCATATGCCTTCAC | |
| | | GAGAGTTACTGGCCCATATAACCGGTATATATAA | |
| | | (SEQ ID NO: 45) | |
| pJL_055 | TET2 | ATGggaggccttGATTACAAGGACGACGACGACAAGgc | MGGLDYKDDDDKASDFPSCRCVEQIIEKD |
| | catalytic | tagcGACTTCCCTTCCTGCAGGTGCGTTGAACAAAT | EGPFYTHLGAGPNVAAREIMEERFGQKG |
| | | AATTGAAAAAGACGAGGGTCCATTTTATACACATC | KAIRIERVIYTGKEGKSSQGCPIAKWVVRR |
| | | TGGGAGCTGGACCAAACGTCGCGGCAATCCGCGA | SSSEEKLLCLVRERAGHTVEAAVIVILILV |
| | | GATAATGGAAGAACGGTTCGGCCAAAAAGGGAAA | WEGIPLSLADKLYSELTETLRKYGTLTNRR |
| | | GCAATAAGAATCGAACGCGTTATTTACACTGGTAA | CALNEERTCACQGLDPETCGASFSFGCSW |
| | | GGAGGGTAAGAGTAGCCAGGGGTGCCCAATAGCT | SMYYNGCKFARSKIPRKFKLLGDDPKEEE |
| | | AAATGGGTCGTCAGAAGGTCCTCAAGTGAAGAGA | KLESHLQNLSTLMAPTYKKLAPDAYNNQI |
| | | AACTCCTCTGCCTTGTCCGAGAAAGGGCTGGCCAC | EYEHRAPECRLGLKEGRPFSGVTACLDFC |
| | | ACTTGTGAGGCAGCCGTCATCGTCATCTTGATTCT | AHAHRDLHNMQNGSTLVCTLTREDNREF |
| | | GGTCTGGGAGGGGATCCCCCTTAGTCTCGCAGACA | GGKPEDEQLHVLPLYKVSDVDEFGSVEAQ |
| | | AACTCTATTCCGAGCTCACCGAAACGCTTCGCAAA | EEKKRSGAIQVLSSFRRKVRMLAEPVKTC |
| | | TATGGGACTCTGACAAATAGGAGGTGCGCCCTGA | RQRKLEAKKAAAEKLSSLENSSNKNEKEK |
| | | ATGAAGAGAGAACATGCGCATGCCAAGGCTTGGA | SAPSRTKQTENASQAKQLAELLRLSGPVM |
| | | TCCCGAGACCTGTGGTGCCAGCTTTTCCTTTGGAT | QQSQQPQPLQKQPPQPQQQQRPQQQQPH |
| | | GTAGCTGGAGTATGTACTACAACGGCTGTAAGTTT | HPQTESVNSYSASGSTNPYMRRPNPVSPYP |
| | | GCAAGGTCCAAAATTCCTCGCAAATTTAAGTTGCT | NSSHTSDIYGSTSPMNFYSTSSQAAGSYLN |
| | | CGGCGACGACCCTAAGGAAGAAGAAAAGCTCGAG | SSNPMNPYPGLLNQNTQYPSYQCNGNLSV |
| | | AGTCATCTCCAAAACTTGAGTACTCTCATGGCCCC | DNCSPYLGSYSPQSQPMDLYRYPSQDPLS |
| | | TACATATAAGAAACTGGCTCCGGATGCATATAAC | KLSLPPIHTLYQPRFGNSQSFTSKYLGYGN |
| | | AATCAGATAGAATATGAGCACAGGGCACCCGAGT | QNMQGDGFSSCTIRPNVHHVGKLPPYPTH |
| | | GCAGACTGGGATTGAAGGAAGGGAGGCCTTTTTC | EMDGHFMGATSRLPPNLSNPNMDYKNGE |
| | | AGGTGTAACGGCTTGCCTCGACTTTTGTGCTCACG | HHSPSHIIHNYSAAPGMFNSSLHALHLQNK |
| | | CTCACAGGGATCTGCACAACATGCAGAACGGCAG | ENDMLSHTANGLSKMLPALNHDRTACVQ |
| | | CACACTCGTTTGTACTCTGACCAGAGAAGACAATC | GGLHKLSDANGQEKQPLALVQGVASGAE |
| | | GAGAGTTCGGTGGGAAACCTGAGGACGAACAACT | DNDEVWSDSEQSFLDPDIGGVAVAPTHGS |
| | | TCACGTCTTGCCACTGTACAAAGTCTCTGATGTTG | ILIECAKRELHATTPLKNPNRNHPTRISLVF |
| | | ATGAATTTGGGTCAGTTGAAGCCCAAGAGGAAAA | YQHKSMNEPKHGLALWEAKMAEKAREK |
| | | AAAACGATCAGGGGCGATCCAAGTACTGTCATCA | EEECEKYGPDYVPQKSHGKKVKREPAEPH |
| | | TTTCGGAGAAAGGTAAGAATGCTTGCCGAGCCTGT | ETSEPTYLRFIKSLAERTMSVTTDSTVTTSP |
| | | CAAAACGTGCCGCCAACGCAAACTGGAGGCCAAA | YAFTRVTGPYNRYI (SEQ ID NO: 48) |
| | | AAGGCAGCAGCAGAAAAGTTGAGTTCTCTTGAGA | |
| | | ACAGCTCAAACAAAAACGAGAAAGAAAAGAGCG | |
| | | CACCGTCACGGACAAAGCAGACAGAAAACGCTTC | |
| | | ACAAGCGAAGCAGCTCGCAGAACTCCTTCGGTTGT | |
| | | CAGGCCCCGTCATGCAACAAAGTCAACAGCCCCA | |
| | | GCCGCTGCAGAAACAGCCACCCCAGCCCCAACAG | |
| | | CAACAGAGACCGCAACAACAACAGCCCCATCACC | |
| | | CGCAGACAGAAAGCGTAAATAGCTATTCCGCGTC | |
| | | AGGGTCCACCAACCCTTATATGCGGAGGCCCAATC | |
| | | CTGTGAGTCCATATCCCAATTCCAGTCACACTTCC | |
| | | GATATCTATGGGAGCACAAGCCCCATGAATTTTTA | |
| | | CTCTACTTCATCTCAGGCCGCAGGTTCATATCTTA | |
| | | ATAGCTCCAATCCGATGAATCCCTATCCCGGCCTC | |
| | | CTTAATCAGAATACCCAGTACCCATCATACCAATG | |
| | | CAACGGAAACCTGTCAGTTGATAACTGCTCCCCGT | |
| | | ACCTTGGAAGCTACTCCCCACAGTCCCAGCCCATG | |
| | | GATCTGTACCGGTATCCAAGTCAGGACCCACTGTC | |
| | | TAAATTGAGTCTGCCGCCAATTCACACTCTTTATC | |
| | | AACCGAGATTTGGAAACAGCCAGTCATTCACATCT | |
| | | AAATACCTGGGTTATGGAAATCAGAATATGCAAG | |
| | | GAGATGGATTCTCCTCATGTACCATTCGACCAAAC | |
| | | GTTCACCATGTTGGCAAGCTCCCACCTTACCCCAC | |
| | | CCATGAAATGGATGGTCATTTCATGGGAGCGACcT | |
| | | CTCGATTGCCGCCAAATCTGAGTAATCCTAACATG | |
| | | GATTATAAAAATGGTGAACACCATAGCCCTTCCCA | |
| | | TATTATCCATAACTATTCTGCAGCCCCAGGGATGT | |
| | | TTAACTCTTCATTGCACGCTTTGCATTTGCAGAAC | |
| | | AAGGAGAATGATATGCTGTCCCATACCGCAAACG | |
| | | GACTCAGTAAAATGCTTCCAGCGCTTAATCATGAC | |
| | | CGGACAGCGTGCGTCCAAGGTGGGCTTCACAAGC | |
| | | TTTCCGACGCCAACGGTCAGGAGAAGCAGCCTCT | |
| | | GGCTTTGGTCCAGGGGGTGGCGTCCGGAGCAGAA | |
| | | GACAACGACGAAGTATGGAGCGATAGCGAGCAGA | |
| | | GCTTCCTGGATCCTGACATTGGGGGCGTGGCCGTG | |
| | | GCACCGACCCATGGCTCTATCCTTATAGAGTGCGC | |
| | | GAAACGAGAGCTCCACGCCACCACACCGCTTAAG | |
| | | AATCCCAATCGCAATCATCCAACACGCATCAGTCT | |
| | | TGTATTTTATCAACACAAGTCCATGAATGAGCCCA | |
| | | AGCACGGTCTGGCCCTTTGGGAAGCTAAAATGGCT | |
| | | GAAAAAGCGAGGGAGAAGGAAGAAGAATGCGAA | |
| | | AAGTATGGGCCCGATTACGTTCCTCAAAAAAGTCA | |
| | | TGGAAAAAAAGTCAAGCGCGAGCCAGCTGAACCT | |
| | | CACGAGACTTCAGAACCTACGTACTTGAGATTCAT | |
| | | TAAGTCTCTTGCTGAACGGACCATGTCTGTAACCA | |
| | | CAGACTCAACGGTTACCACCTCTCCATATGCCTTC | |
| | | ACGAGAGTTACTGGCCCATATAACCGGTATATA | |
| | | (SEQ ID NO: 47) | |
| pJL_056 | DNMT3A | AAGGGCGGCGGCGGCTCCGGCGGCGGCGGCTCCA | KGGGGSGGGGSNHDQEFDPPKVYPPVPAE |
| | catalytic | ATCATGACCAAGAATTTGATCCACCTAAGGTATAT | KRKPIRVLSLFDGIATGLLVLKDLGIQVDR |
| | | CCCCCTGTGCCAGCAGAAAAAAGAAAGCCCATTC | YIASEVCEDSITVGMVRHQGKIMYVGDVR |
| | | GGGTCCTTAGCCTCTTCGACGGGATAGCCACCGGG | SVTQKHIQEWGPFDLVIGGSPCNDLSIVNP |
| | | CTCCTTGTCCTGAAAGACCTTGGCATACAGGTAGA | ARKGLYEGTGRLFFEFYRLLHDARPKEGD |
| | | TCGGTATATCGCTTCCGAGGTCTGTGAAGACTCAA | DRPFFWLFENVVAMGVSDKRDISRFLESN |
| | | TCACCGTTGGGATGGTGCGCCACCAGGGCAAAAT | PVMIDAKEVSAAHRARYFWGNLPGMNRP |
| | | TATGTACGTTGGAGATGTCCGATCAGTCACTCAGA | LASTVNDKLELQECLEHGRIAKFSKVRTIT |
| | | AACATATCCAAGAGTGGGGGCCCTTTGACCTCGTC | TRSNSIKQGKDQHFPVFMNEKEDILWCTE |
| | | ATCGGTGGGTCCCCCTGCAATGACTTGTCAATAGT | MERVFGFPVHYTDVSNMSRLARQRLLGR |
| | | CAACCCGGCGCGGAAGGGTCTCTACGAAGGTACC | SWSVPVIRHLFAPLKEYFACV* (SEQ ID |
| | | GGGAGGCTCTTTTTTGAATTTTATAGGCTCCTTCAT | NO: 50) |
| | | GATGCTAGACCGAAGGAAGGAGATGATCGACCAT | |
| | | TTTTCTGGCTGTTTGAGAATGTGGTTGCCATGGGT | |
| | | GTGTCAGATAAGCGGGATATATCAAGATTCCTCGA | |
| | | GAGCAACCCGGTAATGATTGACGCCAAAGAAGTA | |
| | | AGCGCGGCACACCGAGCTAGGTACTTCTGGGGAA | |
| | | ACCTGCCGGGAATGAATAGACCCTTGGCCAGCAC | |
| | | AGTAAATGACAAACTCGAGTTGCAAGAGTGTCTT | |
| | | GAGCACGGTCGCATAGCTAAATTCAGTAAAGTAA | |
| | | GGACGATCACCACTCGGAGTAATAGTATTAAGCA | |
| | | AGGGAAGGATCAGCATTTCCCAGTGTTCATGAAC | |
| | | GAAAAGGAGGACATCCTTTGGTGCACCGAAATGG | |
| | | AGAGAGTATTCGGGTTCCCAGTTCATTACACAGAT | |
| | | GTGAGCAATATGTCACGACTGGCTCGGCAACGGC | |
| | | TCCTGGGGAGATCATGGAGTGTCCCGGTTATTAGA | |
| | | CATCTCTTTGCTCCTCTCAAAGAATACTTCGCTTGT | |
| | | GTGTAA (SEQ ID NO: 49) | |
| pJL_057 | DNMT3L- | ATGggaagcGAGAATCCCTTGGAAATGTTTGAAACC | MGSENPLEMFETVPVWRRQPVRVLSLFED |
| | SOCS2 | GTGCCTGTTTGGAGAAGACAACCGGTGCGCGTACT | IKKELTSLGFLESGSDPGQLKHVVDVTDT |
| | | TTCATTGTTCGAAGACATTAAAAAGGAGCTCACCA | VRKDVEEWGPFDLVYGATPPLGHTCDRPP |
| | | GCCTTGGGTTCCTCGAGAGTGGAAGCGATCCTGGT | SWYLFQFHRLLQYARPKPGSPRPFFWMFV |
| | | CAGTTGAAACACGTGGTTGACGTCACAGACACGG | DNLVLNKEDLDVASRFLEMEPVTIPDVHG |
| | | TGAGGAAGGATGTTGAGGAGTGGGGTCCATTCGA | GSLQNAVRVWSNIPAIRSRHWALVSEEEL |
| | | TTTGGTCTATGGCGCAACCCCTCCGCTTGGACATA | SLLAQNKQSSKLAAKWPTKLVKNCFLPLR |
| | | CGTGCGATAGACCTCCGAGCTGGTATCTTTTCCAG | EYFKYFSTELTSSLGSGSGSGAPRNGTVHL |
| | | TTTCACAGGCTGCTTCAATACGCAAGACCGAAGCC | YLTKPLYTSAPSLQHLCRLTINKCTGAIWG |
| | | GGGTTCCCCTCGGCCCTTCTTCTGGATGTTTGTCGA | LPLPTRLKDYLEEYKFQV (SEQ ID NO: 52) |
| | | TAATCTCGTTTTGAATAAAGAGGATTTGGATGTAG | |
| | | CATCCCGATTCCTTGAAATGGAACCGGTAACGATC | |
| | | CCCGATGTTCATGGTGGGAGCTTGCAGAACGCTGT | |
| | | CCGGGTGTGGAGTAACATACCTGCCATCCGGTCAA | |
| | | GGCACTGGGCTCTCGTAAGTGAGGAAGAACTCTC | |
| | | ACTGCTGGCGCAGAACAAGCAGTCTTCTAAATTGG | |
| | | CGGCAAAATGGCCCACCAAGCTTGTGAAAAATTG | |
| | | CTTTCTGCCCCTTCGAGAGTATTTTAAGTACTTTAG | |
| | | TACTGAACTCACATCATCACTGGGCAGTGGTAGTG | |
| | | GCtccggaGCTCCACGAAACGGCACGGTCCACCTTTA | |
| | | CTTGACGAAACCGCTTTATACCTCCGCACCGTCCC | |
| | | TCCAGCACCTGTGTCGGCTGACAATCAATAAGTGT | |
| | | ACAGGTGCGATATGGGGATTGCCGCTTCCGACCCG | |
| | | CCTTAAAGATTACCTCGAGGAATACAAATTTCAAG | |
| | | TC (SEQ ID NO: 51) | |
| pJL_058 | DNMT3L- | ATGggaggccttGATTACAAGGACGACGACGACAAGgc | MGGLDYKDDDDKASENPLEMFETVPVWR |
| | VPX | tagcGAGAATCCCTTGGAAATGTTTGAAACCGTGCC | RQPVRVLSLFEDIKKELTSLGFLESGSDPG |
| | | TGTTTGGAGAAGACAACCGGTGCGCGTACTTTCAT | QLKHVVDVTDTVRKDVEEWGPFDLVYGA |
| | | TGTTCGAAGACATTAAAAAGGAGCTCACCAGCCTT | TPPLGHTCDRPPSWYLFQFHRLLQYARPK |
| | | GGGTTCCTCGAGAGTGGAAGCGATCCTGGTCAGTT | PGSPRPFFWMFVDNLVLNKEDLDVASRFL |
| | | GAAACACGTGGTTGACGTCACAGACACGGTGAGG | EMEPVTIPDVHGGSLQNAVRVWSNIPAIRS |
| | | AAGGATGTTGAGGAGTGGGGTCCATTCGATTTGGT | RHWALVSEEELSLLAQNKQSSKLAAKWP |
| | | CTATGGCGCAACCCCTCCGCTTGGACATACGTGCG | TKLVKNCFLPLREYFKYFSTELTSSLGSGS |
| | | ATAGACCTCCGAGCTGGTATCTTTTCCAGTTTCAC | GSGADPRERVPPGNSGEETIGEAFEWLDR |
| | | AGGCTGCTTCAATACGCAAGACCGAAGCCGGGTT | TIEALNREAVNHLPRELIFQVWQRSWAYW |
| | | CCCCTCGGCCCTTCTTCTGGATGTTTGTCGATAATC | HDEQGMSTSYTKYRYLCIMQKAVYIHFK |
| | | TCGTTTTGAATAAAGAGGATTTGGATGTAGCATCC | KGCTCLGRGHGPGGWRPGPPPPPPPGLV |
| | | CGATTCCTTGAAATGGAACCGGTAACGATCCCCGA | (SEQ ID NO: 54) |
| | | TGTTCATGGTGGGAGCTTGCAGAACGCTGTCCGGG | |
| | | TGTGGAGTAACATACCTGCCATCCGGTCAAGGCAC | |
| | | TGGGCTCTCGTAAGTGAGGAAGAACTCTCACTGCT | |
| | | GGCGCAGAACAAGCAGTCTTCTAAATTGGCGGCA | |
| | | AAATGGCCCACCAAGCTTGTGAAAAATTGCTTTCT | |
| | | GCCCCTTCGAGAGTATTTTAAGTACTTTAGTACTG | |
| | | AACTCACATCATCACTGGGCAGTGGTAGTGGCtccg | |
| | | gaGCAGACCCGAGGGAAAGAGTTCCACCAGGTAAC | |
| | | AGTGGCGAAGAAACGATCGGCGAAGCGTTCGAGT | |
| | | GGCTTGATCGGACTATTGAGGCTCTTAATCGGGAG | |
| | | GCTGTGAACCACCTGCCCCGGGAACTTATTTTCCA | |
| | | GGTCTGGCAGAGAAGTTGGGCCTACTGGCACGAT | |
| | | GAGCAAGGCATGTCCACATCCTATACAAAGTATC | |
| | | GGTATCTTTGTATAATGCAGAAGGCTGTATATATC | |
| | | CACTTTAAGAAGGGATGTACTTGTCTGGGTAGAGG | |
| | | TCATGGACCAGGAGGGTGGAGACCAGGCCCTCCA | |
| | | CCTCCTCCACCTCCAGGTCTTGTG (SEQ ID NO: 53) | |
| pJL_059 | 913iK0- | ATGggaggccttGATTACAAGGACGACGACGACAAGgc | MGGLDYKDDDDKASFNVLMVHRRSHTG |
| | DNMT3L | tagcTTCAATGTCTTAATGGTTCATcggCGAAGCCATA | ERPLQCEICGFTCRQRGNLLRHIRLHTGER |
| | | CTGGTGAACGCCCATTGCAGTGCGAAATATGCGG | PFRCHLCNYACQRRDALSGSGSGSGENPL |
| | | CTTTACCTGCCGCCAGcgcGGTAACCTCCTCCGCCA | EMFETVPVWRRQPVRVLSLFEDIKKELTSL |
| | | CATTcgtCTGCACACAGGGGAAcggCCTTTTcggTGTC | GFLESGSDPGQLKHVVDVTDTVRKDVEE |
| | | ACCTCTGCAACTATGCATGCCAAAGAAGAGATGC | WGPFDLVYGATPPLGHTCDRPPSWYLFQF |
| | | GCTCAGCGGGTCAGGTAGTGGCtccggaGAGAATCC | HRLLQYARPKPGSPRPFFWMFVDNLVLNK |
| | | CTTGGAAATGTTTGAAACCGTGCCTGTTTGGAGAA | EDLDVASRFLEMEPVTIPDVHGGSLQNAV |
| | | GACAACCGGTGCGCGTACTTTCATTGTTCGAAGAC | RVWSNIPAIRSRHWALVSEEELSLLAQNK |
| | | ATTAAAAAGGAGCTCACCAGCCTTGGGTTCCTCGA | QSSKLAAKWPTKLVKNCFLPLREYFKYFS |
| | | GAGTGGAAGCGATCCTGGTCAGTTGAAACACGTG | TELTSSL (SEQ ID NO: 56) |
| | | GTTGACGTCACAGACACGGTGAGGAAGGATGTTG | |
| | | AGGAGTGGGGTCCATTCGATTTGGTCTATGGCGCA | |
| | | ACCCCTCCGCTTGGACATACGTGCGATAGACCTCC | |
| | | GAGCTGGTATCTTTTCCAGTTTCACAGGCTGCTTC | |
| | | AATACGCAAGACCGAAGCCGGGTTCCCCTCGGCC | |
| | | CTTCTTCTGGATGTTTGTCGATAATCTCGTTTTGAA | |
| | | TAAAGAGGATTTGGATGTAGCATCCCGATTCCTTG | |
| | | AAATGGAACCGGTAACGATCCCCGATGTTCATGGT | |
| | | GGGAGCTTGCAGAACGCTGTCCGGGTGTGGAGTA | |
| | | ACATACCTGCCATCCGGTCAAGGCACTGGGCTCTC | |
| | | GTAAGTGAGGAAGAACTCTCACTGCTGGCGCAGA | |
| | | ACAAGCAGTCTTCTAAATTGGCGGCAAAATGGCC | |
| | | CACCAAGCTTGTGAAAAATTGCTTTCTGCCCCTTC | |
| | | GAGAGTATTTTAAGTACTTTAGTACTGAACTCACA | |
| | | TCATCACTG (SEQ ID NO: 55) | |
| pJL_060 | DNMT3L(K0)- | ATGggaagcGAGAATCCCTTGGAAATGTTTGAAACC | MGSENPLEMFETVPVWRRQPVRVLSLFED |
| | SOCS2 | GTGCCTGTTTGGAGAAGACAACCGGTGCGCGTACT | IRRELTSLGFLESGSDPGQLRHVVDVTDTV |
| | | TTCATTGTTCGAAGACATTAgAAgGGAGCTCACCA | RRDVEEWGPFDLVYGATPPLGHTCDRPPS |
| | | GCCTTGGGTTCCTCGAGAGTGGAAGCGATCCTGGT | WYLFQFHRLLQYARPRPGSPRPFFWMFVD |
| | | CAGTTGAgACACGTGGTTGACGTCACAGACACGGT | NLVLNREDLDVASRFLEMEPVTIPDVHGG |
| | | GAGGcgGGATGTTGAGGAGTGGGGTCCATTCGATT | SLQNAVRVWSNIPAIRSRHWALVSEEELSL |
| | | TGGTCTATGGCGCAACCCCTCCGCTTGGACATACG | LAQNRQSSRLAARWPTRLVRNCFLPLREY |
| | | TGCGATAGACCTCCGAGCTGGTATCTTTTCCAGTT | FRYFSTELTSSLGSGSGSGAPRNGTVHLYL |
| | | TCACAGGCTGCTTCAATACGCAAGACCGAgGCCGG | TKPLYTSAPSLQHLCRLTINKCTGAIWGLP |
| | | GTTCCCCTCGGCCCTTCTTCTGGATGTTTGTCGATA | LPTRLKDYLEEYKFQV (SEQ ID NO: 58) |
| | | ATCTCGTTTTGAATAgAGAGGATTTGGATGTAGCA | |
| | | TCCCGATTCCTTGAAATGGAACCGGTAACGATCCC | |
| | | CGATGTTCATGGTGGGAGCTTGCAGAACGCTGTCC | |
| | | GGGTGTGGAGTAACATACCTGCCATCCGGTCAAG | |
| | | GCACTGGGCTCTCGTAAGTGAGGAAGAACTCTCA | |
| | | CTGCTGGCGCAGAACcgGCAGTCTTCTAggTTGGCG | |
| | | GCAAgATGGCCCACCcgGCTTGTGAgAAATTGCTTT | |
| | | CTGCCCCTTCGAGAGTATTTTAgGTACTTTAGTACT | |
| | | GAACTCACATCATCACTGGGCAGTGGTAGTGGCtcc | |
| | | ggaGCTCCACGAAACGGCACGGTCCACCTTTACTTG | |
| | | ACGAAACCGCTTTATACCTCCGCACCGTCCCTCCA | |
| | | GCACCTGTGTCGGCTGACAATCAATAAGTGTACAG | |
| | | GTGCGATATGGGGATTGCCGCTTCCGACCCGCCTT | |
| | | AAAGATTACCTCGAGGAATACAAATTTCAAGTC | |
| | | (SEQ ID NO: 57) | |
| pJL_061 | DNMT3L(K0)- | ATGggaagcGAGAATCCCTTGGAAATGTTTGAAACC | MGSENPLEMFETVPVWRRQPVRVLSLFED |
| | VPX | GTGCCTGTTTGGAGAAGACAACCGGTGCGCGTACT | IRRELTSLGFLESGSDPGQLRHVVDVTDTV |
| | | TTCATTGTTCGAAGACATTAgAAgGGAGCTCACCA | RRDVEEWGPFDLVYGATPPLGHTCDRPPS |
| | | GCCTTGGGTTCCTCGAGAGTGGAAGCGATCCTGGT | WYLFQFHRLLQYARPRPGSPRPFFWMFVD |
| | | CAGTTGAgACACGTGGTTGACGTCACAGACACGGT | NLVLNREDLDVASRFLEMEPVTIPDVHGG |
| | | GAGGcgGGATGTTGAGGAGTGGGGTCCATTCGATT | SLQNAVRVWSNIPAIRSRHWALVSEEELSL |
| | | TGGTCTATGGCGCAACCCCTCCGCTTGGACATACG | LAQNRQSSRLAARWPTRLVRNCFLPLREY |
| | | TGCGATAGACCTCCGAGCTGGTATCTTTTCCAGTT | FRYFSTELTSSLGSGSGSGADPRERVPPGN |
| | | TCACAGGCTGCTTCAATACGCAAGACCGAgGCCGG | SGEETIGEAFEWLDRTIEALNREAVNHLPR |
| | | GTTCCCCTCGGCCCTTCTTCTGGATGTTTGTCGATA | ELIFQVWQRSWAYWHDEQGMSTSYTKYR |
| | | ATCTCGTTTTGAATAgAGAGGATTTGGATGTAGCA | YLCIMQKAVYIHFKKGCTCLGRGHGPGG |
| | | TCCCGATTCCTTGAAATGGAACCGGTAACGATCCC | WRPGPPPPPPPGLV (SEQ ID NO: 60) |
| | | CGATGTTCATGGTGGGAGCTTGCAGAACGCTGTCC | |
| | | GGGTGTGGAGTAACATACCTGCCATCCGGTCAAG | |
| | | GCACTGGGCTCTCGTAAGTGAGGAAGAACTCTCA | |
| | | CTGCTGGCGCAGAACcgGCAGTCTTCTAggTTGGCG | |
| | | GCAAgATGGCCCACCcgGCTTGTGAgAAATTGCTTT | |
| | | CTGCCCCTTCGAGAGTATTTTAgGTACTTTAGTACT | |
| | | GAACTCACATCATCACTGGGCAGTGGTAGTGGCtcc | |
| | | ggaGCAGACCCGAGGGAAAGAGTTCCACCAGGTAA | |
| | | CAGTGGCGAAGAAACGATCGGCGAAGCGTTCGAG | |
| | | TGGCTTGATCGGACTATTGAGGCTCTTAATCGGGA | |
| | | GGCTGTGAACCACCTGCCCCGGGAACTTATTTTCC | |
| | | AGGTCTGGCAGAGAAGTTGGGCCTACTGGCACGA | |
| | | TGAGCAAGGCATGTCCACATCCTATACAAAGTATC | |
| | | GGTATCTTTGTATAATGCAGAAGGCTGTATATATC | |
| | | CACTTTAAGAAGGGATGTACTTGTCTGGGTAGAGG | |
| | | TCATGGACCAGGAGGGTGGAGACCAGGCCCTCCA | |
| | | CCTCCTCCACCTCCAGGTCTTGTG (SEQ ID NO: 59) | |
| pJL_062 | 913iK0- | ATGggaagcTTCAATGTCTTAATGGTTCATcggCGAAG | MGSFNVLMVHRRSHTGERPLQCEICGFTC |
| | DNMT3L(K0) | CCATACTGGTGAACGCCCATTGCAGTGCGAAATAT | RQRGNLLRHIRLHTGERPFRCHLCNYACQ |
| | | GCGGCTTTACCTGCCGCCAGcgcGGTAACCTCCTCC | RRDALSGSGSGSGENPLEMFETVPVWRRQ |
| | | GCCACATTcgtCTGCACACAGGGGAAcggCCTTTTcgg | PVRVLSLFEDIRRELTSLGFLESGSDPGQLR |
| | | TGTCACCTCTGCAACTATGCATGCCAAAGAAGAG | HVVDVTDTVRRDVEEWGPFDLVYGATPP |
| | | ATGCGCTCAGCGGGTCAGGTAGTGGCtccggaGAGA | LGHTCDRPPSWYLFQFHRLLQYARPRPGS |
| | | ATCCCTTGGAAATGTTTGAAACCGTGCCTGTTTGG | PRPFFWMFVDNLVLNREDLDVASRFLEME |
| | | AGAAGACAACCGGTGCGCGTACTTTCATTGTTCGA | PVTIPDVHGGSLQNAVRVWSNIPAIRSRH |
| | | AGACATTAgAAgGGAGCTCACCAGCCTTGGGTTCC | WALVSEEELSLLAQNRQSSRLAARWPTRL |
| | | TCGAGAGTGGAAGCGATCCTGGTCAGTTGAgACAC | VRNCFLPLREYFRYFSTELTSSL(SEQ ID |
| | | GTGGTTGACGTCACAGACACGGTGAGGcgGGATGT | NO: 62) |
| | | TGAGGAGTGGGGTCCATTCGATTTGGTCTATGGCG | |
| | | CAACCCCTCCGCTTGGACATACGTGCGATAGACCT | |
| | | CCGAGCTGGTATCTTTTCCAGTTTCACAGGCTGCT | |
| | | TCAATACGCAAGACCGAgGCCGGGTTCCCCTCGGC | |
| | | CCTTCTTCTGGATGTTTGTCGATAATCTCGTTTTGA | |
| | | ATAgAGAGGATTTGGATGTAGCATCCCGATTCCTT | |
| | | GAAATGGAACCGGTAACGATCCCCGATGTTCATG | |
| | | GTGGGAGCTTGCAGAACGCTGTCCGGGTGTGGAG | |
| | | TAACATACCTGCCATCCGGTCAAGGCACTGGGCTC | |
| | | TCGTAAGTGAGGAAGAACTCTCACTGCTGGCGCA | |
| | | GAACcgGCAGTCTTCTAggTTGGCGGCAAgATGGCC | |
| | | CACCcgGCTTGTGAgAAATTGCTTTCTGCCCCTTCG | |
| | | AGAGTATTTTAgGTACTTTAGTACTGAACTCACAT | |
| | | CATCACTG (SEQ ID NO: 61) | |
| pJL_063 | IDAXDBM- | ATGggaggccttGATTACAAGGACGACGACGACAAGgc | MGGLDYKDDDDKASMHHRNDSQRLGKA |
| | SOCS2 | tagcATGCACCACCGGAACGATTCACAAAGGCTGGG | GCPPEPSLQMANTNFLSTLSPEHCRPLAGE |
| | | TAAGGCGGGCTGCCCTCCGGAGCCAAGCCTCCAG | CMNKLKCGAAEAEIMNLPERVGTFSAIPA |
| | | ATGGCAAATACGAATTTTCTTAGTACGCTGTCACC | LGGISLPPGVIVMTALHSPAAASAAVTDSA |
| | | GGAACATTGTAGACCGCTGGCGGGAGAGTGCATG | FQIANLADCPQNHSSSSSSSSGGAGGANPA |
| | | AACAAACTTAAATGCGGAGCTGCTGAAGCCGAGA | KKKRKRCGVCVPCKRLINCGVCSSCRNRK |
| | | TTATGAACCTCCCGGAACGGGTCGGCACTTTCAGT | AGAAICKFRKCEELKKKPGTSLERTPVPSA |
| | | GCTATTCCAGCGCTGGGGGGAATAAGCCTCCCTCC | EAFRWFFGSGSGSGAPRNGTVHLYLTKPL |
| | | GGGAGTAATCGTAATGACTGCGCTTCATTCACCCG | YTSAPSLQHLCRLTINKCTGAIWGLPLPTR |
| | | CTGCAGCAAGCGCCGCGGTGACCGATAGTGCGTTT | LKDYLEEYKFQV (SEQ ID NO: 64) |
| | | CAGATTGCAAACCTTGCTGACTGCCCCCAGAATCA | |
| | | CAGTTCCTCAAGTTCCAGTTCTAGTGGTGGAGCCG | |
| | | GCGGGGCTAACCCCGCCAAGAAAAAACGGAAGAG | |
| | | ATGCGGTGTTTGTGTTCCATGTAAAAGACTGATTA | |
| | | ACTGTGGCGTGTGTAGTTCCTGTCGCAATAGGAAA | |
| | | GCTGGAGCGGCTATATGTAAATTTCGGAAGTGCG | |
| | | AGGAATTGAAAAAAAAGCCCGGAACCAGCCTGGA | |
| | | GCGGACGCCGGTCCCATCTGCAGAAGCCTTTAGGT | |
| | | GGTTCTTCGGCAGTGGTAGTGGCtccggaGCTCCACG | |
| | | AAACGGCACGGTCCACCTTTACTTGACGAAACCGC | |
| | | TTTATACCTCCGCACCGTCCCTCCAGCACCTGTGT | |
| | | CGGCTGACAATCAATAAGTGTACAGGTGCGATAT | |
| | | GGGGATTGCCGCTTCCGACCCGCCTTAAAGATTAC | |
| | | CTCGAGGAATACAAATTTCAAGTC (SEQ ID NO: 63) | |
| pJL_064 | IDAXDBM- | ATGggaggccttGATTACAAGGACGACGACGACAAGgc | MGGLDYKDDDDKASMHHRNDSQRLGKA |
| | VPX | tagcATGCACCACCGGAACGATTCACAAAGGCTGGG | GCPPEPSLQMANTNFLSTLSPEHCRPLAGE |
| | | TAAGGCGGGCTGCCCTCCGGAGCCAAGCCTCCAG | CMNKLKCGAAEAEIMNLPERVGTFSAIPA |
| | | ATGGCAAATACGAATTTTCTTAGTACGCTGTCACC | LGGISLPPGVIVMTALHSPAAASAAVTDSA |
| | | GGAACATTGTAGACCGCTGGCGGGAGAGTGCATG | FQIANLADCPQNHSSSSSSSSGGAGGANPA |
| | | AACAAACTTAAATGCGGAGCTGCTGAAGCCGAGA | KKKRKRCGVCVPCKRLINCGVCSSCRNRK |
| | | TTATGAACCTCCCGGAACGGGTCGGCACTTTCAGT | AGAAICKFRKCEELKKKPGTSLERTPVPSA |
| | | GCTATTCCAGCGCTGGGGGGAATAAGCCTCCCTCC | EAFRWFFGSGSGSGADPRERVPPGNSGEE |
| | | GGGAGTAATCGTAATGACTGCGCTTCATTCACCCG | TIGEAFEWLDRTIEALNREAVNHLPRELIF |
| | | CTGCAGCAAGCGCCGCGGTGACCGATAGTGCGTTT | QVWQRSWAYWHDEQGMSTSYTKYRYLC |
| | | CAGATTGCAAACCTTGCTGACTGCCCCCAGAATCA | IMQKAVYIHFKKGCTCLGRGHGPGGWRP |
| | | CAGTTCCTCAAGTTCCAGTTCTAGTGGTGGAGCCG | GPPPPPPPGLV(SEQ ID NO: 66) |
| | | GCGGGGCTAACCCCGCCAAGAAAAAACGGAAGAG | |
| | | ATGCGGTGTTTGTGTTCCATGTAAAAGACTGATTA | |
| | | ACTGTGGCGTGTGTAGTTCCTGTCGCAATAGGAAA | |
| | | GCTGGAGCGGCTATATGTAAATTTCGGAAGTGCG | |
| | | AGGAATTGAAAAAAAAGCCCGGAACCAGCCTGGA | |
| | | GCGGACGCCGGTCCCATCTGCAGAAGCCTTTAGGT | |
| | | GGTTCTTCGGCAGTGGTAGTGGCtccggaGCAGACCC | |
| | | GAGGGAAAGAGTTCCACCAGGTAACAGTGGCGAA | |
| | | GAAACGATCGGCGAAGCGTTCGAGTGGCTTGATC | |
| | | GGACTATTGAGGCTCTTAATCGGGAGGCTGTGAAC | |
| | | CACCTGCCCCGGGAACTTATTTTCCAGGTCTGGCA | |
| | | GAGAAGTTGGGCCTACTGGCACGATGAGCAAGGC | |
| | | ATGTCCACATCCTATACAAAGTATCGGTATCTTTG | |
| | | TATAATGCAGAAGGCTGTATATATCCACTTTAAGA | |
| | | AGGGATGTACTTGTCTGGGTAGAGGTCATGGACC | |
| | | AGGAGGGTGGAGACCAGGCCCTCCACCTCCTCCA | |
| | | CCTCCAGGTCTTGTG (SEQ ID NO: 65) | |
| pJL_065 | 913iK0- | ATGggaggccttGATTACAAGGACGACGACGACAAGgc | MGGLDYKDDDDKASFNVLMVHRRSHTG |
| | IDAXDBM | tagcTTCAATGTCTTAATGGTTCATcggCGAAGCCATA | ERPLQCEICGFTCRQRGNLLRHIRLHTGER |
| | | CTGGTGAACGCCCATTGCAGTGCGAAATATGCGG | PFRCHLCNYACQRRDALSGSGSGSGMHH |
| | | CTTTACCTGCCGCCAGcgcGGTAACCTCCTCCGCCA | RNDSQRLGKAGCPPEPSLQMANTNFLSTL |
| | | CATTcgtCTGCACACAGGGGAAcggCCTTTTcggTGTC | SPEHCRPLAGECMNKLKCGAAEAEIMNLP |
| | | ACCTCTGCAACTATGCATGCCAAAGAAGAGATGC | ERVGTFSAIPALGGISLPPGVIVMTALHSPA |
| | | GCTCAGCGGGTCAGGTAGTGGCtccggaATGCACCAC | AASAAVTDSAFQIANLADCPQNHSSSSSSS |
| | | CGGAACGATTCACAAAGGCTGGGTAAGGCGGGCT | SGGAGGANPAKKKRKRCGVCVPCKRLIN |
| | | GCCCTCCGGAGCCAAGCCTCCAGATGGCAAATAC | CGVCSSCRNRKAGAAICKFRKCEELKKKP |
| | | GAATTTTCTTAGTACGCTGTCACCGGAACATTGTA | GTSLERTPVPSAEAFRWFF (SEQ ID NO: |
| | | GACCGCTGGCGGGAGAGTGCATGAACAAACTTAA | 68) |
| | | ATGCGGAGCTGCTGAAGCCGAGATTATGAACCTC | |
| | | CCGGAACGGGTCGGCACTTTCAGTGCTATTCCAGC | |
| | | GCTGGGGGGAATAAGCCTCCCTCCGGGAGTAATC | |
| | | GTAATGACTGCGCTTCATTCACCCGCTGCAGCAAG | |
| | | CGCCGCGGTGACCGATAGTGCGTTTCAGATTGCAA | |
| | | ACCTTGCTGACTGCCCCCAGAATCACAGTTCCTCA | |
| | | AGTTCCAGTTCTAGTGGTGGAGCCGGCGGGGCTA | |
| | | ACCCCGCCAAGAAAAAACGGAAGAGATGCGGTGT | |
| | | TTGTGTTCCATGTAAAAGACTGATTAACTGTGGCG | |
| | | TGTGTAGTTCCTGTCGCAATAGGAAAGCTGGAGCG | |
| | | GCTATATGTAAATTTCGGAAGTGCGAGGAATTGA | |
| | | AAAAAAAGCCCGGAACCAGCCTGGAGCGGACGCC | |
| | | GGTCCCATCTGCAGAAGCCTTTAGGTGGTTCTTC | |
| | | (SEQ ID NO: 67) | |
| pJL_066 | IDAXDBM | ATGggaagcATGCACCACCGGAACGATTCACAAAGG | MGSMHHRNDSQRLGRAGCPPEPSLQMAN |
| | (K0)-SOCS2 | CTGGGTcgGGCGGGCTGCCCTCCGGAGCCAAGCCT | TNFLSTLSPEHCRPLAGECMNRLRCGAAE |
| | | CCAGATGGCAAATACGAATTTTCTTAGTACGCTGT | AEIMNLPERVGTFSAIPALGGISLPPGVIVM |
| | | CACCGGAACATTGTAGACCGCTGGCGGGAGAGTG | TALHSPAAASAAVTDSAFQIANLADCPQN |
| | | CATGAACAgACTTAggTGCGGAGCTGCTGAAGCCG | HSSSSSSSSGGAGGANPARRRRRRCGVCV |
| | | AGATTATGAACCTCCCGGAACGGGTCGGCACTTTC | PCRRLINCGVCSSCRNRRAGAAICRFRRCE |
| | | AGTGCTATTCCAGCGCTGGGGGGAATAAGCCTCCC | ELRRRPGTSLERTPVPSAEAFRWFFGSGSG |
| | | TCCGGGAGTAATCGTAATGACTGCGCTTCATTCAC | SGAPRNGTVHLYLTKPLYTSAPSLQHLCR |
| | | CCGCTGCAGCAAGCGCCGCGGTGACCGATAGTGC | LTINKCTGAIWGLPLPTRLKDYLEEYKFQV |
| | | GTTTCAGATTGCAAACCTTGCTGACTGCCCCCAGA | (SEQ ID NO: 70) |
| | | ATCACAGTTCCTCAAGTTCCAGTTCTAGTGGTGGA | |
| | | GCCGGCGGGGCTAACCCCGCCcgGAgAAgACGGAg | |
| | | GAGATGCGGTGTTTGTGTTCCATGTAgAAGACTGA | |
| | | TTAACTGTGGCGTGTGTAGTTCCTGTCGCAATAGG | |
| | | AgAGCTGGAGCGGCTATATGTAggTTTCGGAgGTGC | |
| | | GAGGAATTGAgAAgAcgGCCCGGAACCAGCCTGGA | |
| | | GCGGACGCCGGTCCCATCTGCAGAAGCCTTTAGGT | |
| | | GGTTCTTCGGCAGTGGTAGTGGCtccggaGCTCCACG | |
| | | AAACGGCACGGTCCACCTTTACTTGACGAAACCGC | |
| | | TTTATACCTCCGCACCGTCCCTCCAGCACCTGTGT | |
| | | CGGCTGACAATCAATAAGTGTACAGGTGCGATAT | |
| | | GGGGATTGCCGCTTCCGACCCGCCTTAAAGATTAC | |
| | | CTCGAGGAATACAAATTTCAAGTC (SEQ ID NO: 69) | |
| pJL_067 | IDAXDM(K0)- | ATGggaagcATGCACCACCGGAACGATTCACAAAGG | MGSMHHRNDSQRLGRAGCPPEPSLQMAN |
| | VPX | CTGGGTcgGGCGGGCTGCCCTCCGGAGCCAAGCCT | TNFLSTLSPEHCRPLAGECMNRLRCGAAE |
| | | CCAGATGGCAAATACGAATTTTCTTAGTACGCTGT | AEIMNLPERVGTFSAIPALGGISLPPGVIVM |
| | | CACCGGAACATTGTAGACCGCTGGCGGGAGAGTG | TALHSPAAASAAVTDSAFQIANLADCPQN |
| | | CATGAACAgACTTAggTGCGGAGCTGCTGAAGCCG | HSSSSSSSSGGAGGANPARRRRRRCGVCV |
| | | AGATTATGAACCTCCCGGAACGGGTCGGCACTTTC | PCRRLINCGVCSSCRNRRAGAAICRFRRCE |
| | | AGTGCTATTCCAGCGCTGGGGGGAATAAGCCTCCC | ELRRRPGTSLERTPVPSAEAFRWFFGSGSG |
| | | TCCGGGAGTAATCGTAATGACTGCGCTTCATTCAC | SGADPRERVPPGNSGEETIGEAFEWLDRTI |
| | | CCGCTGCAGCAAGCGCCGCGGTGACCGATAGTGC | EALNREAVNHLPRELIFQVWQRSWAYWH |
| | | GTTTCAGATTGCAAACCTTGCTGACTGCCCCCAGA | DEQGMSTSYTKYRYLCIMQKAVYIHFKK |
| | | ATCACAGTTCCTCAAGTTCCAGTTCTAGTGGTGGA | GCTCLGRGHGPGGWRPGPPPPPPPGLV |
| | | GCCGGCGGGGCTAACCCCGCCcgGAgAAgACGGAg | (SEQ ID NO: 72) |
| | | GAGATGCGGTGTTTGTGTTCCATGTAgAAGACTGA | |
| | | TTAACTGTGGCGTGTGTAGTTCCTGTCGCAATAGG | |
| | | AgAGCTGGAGCGGCTATATGTAggTTTCGGAgGTGC | |
| | | GAGGAATTGAgAAgAcgGCCCGGAACCAGCCTGGA | |
| | | GCGGACGCCGGTCCCATCTGCAGAAGCCTTTAGGT | |
| | | GGTTCTTCGGCAGTGGTAGTGGCtccggaGCAGACCC | |
| | | GAGGGAAAGAGTTCCACCAGGTAACAGTGGCGAA | |
| | | GAAACGATCGGCGAAGCGTTCGAGTGGCTTGATC | |
| | | GGACTATTGAGGCTCTTAATCGGGAGGCTGTGAAC | |
| | | CACCTGCCCCGGGAACTTATTTTCCAGGTCTGGCA | |
| | | GAGAAGTTGGGCCTACTGGCACGATGAGCAAGGC | |
| | | ATGTCCACATCCTATACAAAGTATCGGTATCTTTG | |
| | | TATAATGCAGAAGGCTGTATATATCCACTTTAAGA | |
| | | AGGGATGTACTTGTCTGGGTAGAGGTCATGGACC | |
| | | AGGAGGGTGGAGACCAGGCCCTCCACCTCCTCCA | |
| | | CCTCCAGGTCTTGTG (SEQ ID NO: 71) | |
| pJL_068 | 913- | ATGggaagcTTCAATGTCTTAATGGTTCATcggCGAAG | MGSFNVLMVHRRSHTGERPLQCEICGFTC |
| | IDAXDBM(K0) | CCATACTGGTGAACGCCCATTGCAGTGCGAAATAT | RQRGNLLRHIRLHTGERPFRCHLCNYACQ |
| | | GCGGCTTTACCTGCCGCCAGcgcGGTAACCTCCTCC | RRDALSGSGSGSGMHHRNDSQRLGRAGC |
| | | GCCACATTcgtCTGCACACAGGGGAAcggCCTTTTcgg | PPEPSLQMANTNFLSTLSPEHCRPLAGECM |
| | | TGTCACCTCTGCAACTATGCATGCCAAAGAAGAG | NRLRCGAAEAEIMNLPERVGTFSAIPALGG |
| | | ATGCGCTCAGCGGGTCAGGTAGTGGCtccggaATGCA | ISLPPGVIVMTALHSPAAASAAVTDSAFQI |
| | | CCACCGGAACGATTCACAAAGGCTGGGTcgGGCGG | ANLADCPQNHSSSSSSSSGGAGGANPARR |
| | | GCTGCCCTCCGGAGCCAAGCCTCCAGATGGCAAA | RRRRCGVCVPCRRLINCGVCSSCRNRRAG |
| | | TACGAATTTTCTTAGTACGCTGTCACCGGAACATT | AAICRFRRCEELRRRPGTSLERTPVPSAEA |
| | | GTAGACCGCTGGCGGGAGAGTGCATGAACAgACT | FRWFF(SEQ ID NO: 74) |
| | | TAggTGCGGAGCTGCTGAAGCCGAGATTATGAACC | |
| | | TCCCGGAACGGGTCGGCACTTTCAGTGCTATTCCA | |
| | | GCGCTGGGGGGAATAAGCCTCCCTCCGGGAGTAA | |
| | | TCGTAATGACTGCGCTTCATTCACCCGCTGCAGCA | |
| | | AGCGCCGCGGTGACCGATAGTGCGTTTCAGATTGC | |
| | | AAACCTTGCTGACTGCCCCCAGAATCACAGTTCCT | |
| | | CAAGTTCCAGTTCTAGTGGTGGAGCCGGCGGGGCT | |
| | | AACCCCGCCcgGAgAAgACGGAgGAGATGCGGTGTT | |
| | | TGTGTTCCATGTAgAAGACTGATTAACTGTGGCGT | |
| | | GTGTAGTTCCTGTCGCAATAGGAgAGCTGGAGCGG | |
| | | CTATATGTAggTTTCGGAgGTGCGAGGAATTGAgAA | |
| | | gAcgGCCCGGAACCAGCCTGGAGCGGACGCCGGTC | |
| | | CCATCTGCAGAAGCCTTTAGGTGGTTCTTC (SEQ | |
| | | ID NO: 73) | |
| pJL_069 | IDAX | ATGCACCACCGGAACGATTCACAAAGGCTGGGTA | MHHRNDSQRLGKAGCPPEPSLQMANTNF |
| | | AGGCGGGCTGCCCTCCGGAGCCAAGCCTCCAGAT | LSTLSPEHCRPLAGECMNKLKCGAAEAEI |
| | | GGCAAATACGAATTTTCTTAGTACGCTGTCACCGG | MNLPERVGTFSAIPALGGISLPPGVIVMTA |
| | | AACATTGTAGACCGCTGGCGGGAGAGTGCATGAA | LHSPAAASAAVTDSAFQIANLADCPQNHS |
| | | CAAACTTAAATGCGGAGCTGCTGAAGCCGAGATT | SSSSSSSGGAGGANPAKKKRKRCGVCVPC |
| | | ATGAACCTCCCGGAACGGGTCGGCACTTTCAGTGC | KRLINCGVCSSCRNRKTGHQICKFRKCEEL |
| | | TATTCCAGCGCTGGGGGGAATAAGCCTCCCTCCGG | KKKPGTSLERTPVPSAEAFRWFF (SEQ ID |
| | | GAGTAATCGTAATGACTGCGCTTCATTCACCCGCT | NO: 76) |
| | | GCAGCAAGCGCCGCGGTGACCGATAGTGCGTTTC | |
| | | AGATTGCAAACCTTGCTGACTGCCCCCAGAATCAC | |
| | | AGTTCCTCAAGTTCCAGTTCTAGTGGTGGAGCCGG | |
| | | CGGGGCTAACCCCGCCAAGAAAAAACGGAAGAGA | |
| | | TGCGGTGTTTGTGTTCCATGTAAAAGACTGATTAA | |
| | | CTGTGGCGTGTGTAGTTCCTGTCGCAATAGGAAAA | |
| | | CTGGGCACCAGATATGTAAATTTCGGAAGTGCGA | |
| | | GGAATTGAAAAAAAAGCCCGGAACCAGCCTGGAG | |
| | | CGGACGCCGGTCCCATCTGCAGAAGCCTTTAGGTG | |
| | | GTTCTTC (SEQ ID NO: 75) | |
| pJL_070 | VPR | ATGGAGCAAGCACCGGAAGATCAGGGACCACAGC | MEQAPEDQGPQREPHNEWTLELLEELKNE |
| | | GCGAACCACACAATGAATGGACATTGGAATTGCT | AVRHFPRIWLHGLGQHIYETYGDTWAGV |
| | | TGAGGAGCTTAAAAATGAAGCTGTTAGACACTTCC | EAIIRILQQLLFIHFRIGCRHSRIGVTQQRRA |
| | | CAAGAATCTGGCTGCACGGCTTGGGTCAGCACAT | RNGASRS (SEQ ID NO: 78) |
| | | ATATGAAACGTATGGAGATACATGGGCAGGTGTC | |
| | | GAGGCTATCATCAGGATCCTTCAGCAGCTCCTCTT | |
| | | CATTCATTTCAGAATAGGGTGTCGACATAGCAGAA | |
| | | TAGGGGTTACCCAGCAACGCAGGGCGCGGAATGG | |
| | | TGCCAGTAGGAGT (SEQ ID NO: 77) | |
| pJL_084 | 913- | ATGTCTGGGTTCAATGTGCTGATGGTCCACAAGAG | MSGFNVLMVHKRSHTGERPLQCEICGFTC |
| | DNMT3L- | AAGTCACACCGGCGAGCGGCCCCTGCAGTGCGAA | RQKGNLLRHIKLHTGEKPFKCHLCNYACQ |
| | SOCS2 | ATCTGTGGATTTACATGCCGCCAAAAAGGGAACTT | RRDALGSGSGSGSGGENPLEMFETVPVWR |
| | | GCTTCGACACATAAAGCTGCATACCGGCGAGAAG | RQPVRVLSLFEDIKKELTSLGFLESGSDPG |
| | | CCATTTAAGTGCCACTTGTGTAACTACGCTTGTCA | QLKHVVDVTDTVRKDVEEWGPFDLVYGA |
| | | GAGGCGGGACGCATTGGGGAGCGGATCTGGATCA | TPPLGHTCDRPPSWYLFQFHRLLQYARPK |
| | | GGAAGCGGGGGAGAGAATCCCTTGGAAATGTTTG | PGSPRPFFWMFVDNLVLNKEDLDVASRFL |
| | | AAACCGTGCCTGTTTGGAGAAGACAACCGGTGCG | EMEPVTIPDVHGGSLQNAVRVWSNIPAIRS |
| | | CGTACTTTCATTGTTCGAAGACATTAAAAAGGAGC | RHWALVSEEELSLLAQNKQSSKLAAKWP |
| | | TCACCAGCCTTGGGTTCCTCGAGAGTGGAAGCGAT | TKLVKNCFLPLREYFKYFSTELTSSLGSGS |
| | | CCTGGTCAGTTGAAACACGTGGTTGACGTCACAGA | GSGAPRNGTVHLYLTKPLYTSAPSLQHLC |
| | | CACGGTGAGGAAGGATGTTGAGGAGTGGGGTCCA | RLTINKCTGAIWGLPLPTRLKDYLEEYKFQ |
| | | TTCGATTTGGTCTATGGCGCAACCCCTCCGCTTGG | V (SEQ ID NO: 80) |
| | | ACATACGTGCGATAGACCTCCGAGCTGGTATCTTT | |
| | | TCCAGTTTCACAGGCTGCTTCAATACGCAAGACCG | |
| | | AAGCCGGGTTCCCCTCGGCCCTTCTTCTGGATGTT | |
| | | TGTCGATAATCTCGTTTTGAATAAAGAGGATTTGG | |
| | | ATGTAGCATCCCGATTCCTTGAAATGGAACCGGTA | |
| | | ACGATCCCCGATGTTCATGGTGGGAGCTTGCAGAA | |
| | | CGCTGTCCGGGTGTGGAGTAACATACCTGCCATCC | |
| | | GGTCAAGGCACTGGGCTCTCGTAAGTGAGGAAGA | |
| | | ACTCTCACTGCTGGCGCAGAACAAGCAGTCTTCTA | |
| | | AATTGGCGGCAAAATGGCCCACCAAGCTTGTGAA | |
| | | AAATTGCTTTCTGCCCCTTCGAGAGTATTTTAAGT | |
| | | ACTTTAGTACTGAACTCACATCATCACTGGGCAGT | |
| | | GGTAGTGGCtccggaGCTCCACGAAACGGCACGGTC | |
| | | CACCTTTACTTGACGAAACCGCTTTATACCTCCGC | |
| | | ACCGTCCCTCCAGCACCTGTGTCGGCTGACAATCA | |
| | | ATAAGTGTACAGGTGCGATATGGGGATTGCCGCTT | |
| | | CCGACCCGCCTTAAAGATTACCTCGAGGAATACA | |
| | | AATTTCAAGTC (SEQ ID NO: 79) | |
| pJL_085 | DNMT3L- | ATGggaagcGAGAATCCCTTGGAAATGTTTGAAACC | MGSENPLEMFETVPVWRRQPVRVLSLFED |
| | SOCS2-913 | GTGCCTGTTTGGAGAAGACAACCGGTGCGCGTACT | IKKELTSLGFLESGSDPGQLKHVVDVTDT |
| | | TTCATTGTTCGAAGACATTAAAAAGGAGCTCACCA | VRKDVEEWGPFDLVYGATPPLGHTCDRPP |
| | | GCCTTGGGTTCCTCGAGAGTGGAAGCGATCCTGGT | SWYLFQFHRLLQYARPKPGSPRPFFWMFV |
| | | CAGTTGAAACACGTGGTTGACGTCACAGACACGG | DNLVLNKEDLDVASRFLEMEPVTIPDVHG |
| | | TGAGGAAGGATGTTGAGGAGTGGGGTCCATTCGA | GSLQNAVRVWSNIPAIRSRHWALVSEEEL |
| | | TTTGGTCTATGGCGCAACCCCTCCGCTTGGACATA | SLLAQNKQSSKLAAKWPTKLVKNCFLPLR |
| | | CGTGCGATAGACCTCCGAGCTGGTATCTTTTCCAG | EYFKYFSTELTSSLGSGSGSGSGGFNVLMV |
| | | TTTCACAGGCTGCTTCAATACGCAAGACCGAAGCC | HKRSHTGERPLQCEICGFTCRQKGNLLRHI |
| | | GGGTTCCCCTCGGCCCTTCTTCTGGATGTTTGTCGA | KLHTGEKPFKCHLCNYACQRRDALGSGS |
| | | TAATCTCGTTTTGAATAAAGAGGATTTGGATGTAG | GSGAPRNGTVHLYLTKPLYTSAPSLQHLC |
| | | CATCCCGATTCCTTGAAATGGAACCGGTAACGATC | RLTINKCTGAIWGLPLPTRLKDYLEEYKFQ |
| | | CCCGATGTTCATGGTGGGAGCTTGCAGAACGCTGT | V (SEQ ID NO: 82) |
| | | CCGGGTGTGGAGTAACATACCTGCCATCCGGTCAA | |
| | | GGCACTGGGCTCTCGTAAGTGAGGAAGAACTCTC | |
| | | ACTGCTGGCGCAGAACAAGCAGTCTTCTAAATTGG | |
| | | CGGCAAAATGGCCCACCAAGCTTGTGAAAAATTG | |
| | | CTTTCTGCCCCTTCGAGAGTATTTTAAGTACTTTAG | |
| | | TACTGAACTCACATCATCACTGggcagtggctcgggctcggg | |
| | | gtccggcggaTTCAATGTCTTAATGGTTCATAAGCGAA | |
| | | GCCATACTGGTGAACGCCCATTGCAGTGCGAAAT | |
| | | ATGCGGCTTTACCTGCCGCCAGAAAGGTAACCTCC | |
| | | TCCGCCACATTAAACTGCACACAGGGGAAAAACC | |
| | | TTTTAAGTGTCACCTCTGCAACTATGCATGCCAAA | |
| | | GAAGAGATGCGCTCGGCAGTGGTAGTGGCtccggaG | |
| | | CTCCACGAAACGGCACGGTCCACCTTTACTTGACG | |
| | | AAACCGCTTTATACCTCCGCACCGTCCCTCCAGCA | |
| | | CCTGTGTCGGCTGACAATCAATAAGTGTACAGGTG | |
| | | CGATATGGGGATTGCCGCTTCCGACCCGCCTTAAA | |
| | | GATTACCTCGAGGAATACAAATTTCAAGTC SEQ | |
| | | ID NO: 81) | |
| pJL_086 | DNMT3L- | ATGggaagcGAGAATCCCTTGGAAATGTTTGAAACC | MGSENPLEMFETVPVWRRQPVRVLSLFED |
| | 913-SOCS2 | GTGCCTGTTTGGAGAAGACAACCGGTGCGCGTACT | IKKELTSLGFLESGSDPGQLKHVVDVTDT |
| | | TTCATTGTTCGAAGACATTAAAAAGGAGCTCACCA | VRKDVEEWGPFDLVYGATPPLGHTCDRPP |
| | | GCCTTGGGTTCCTCGAGAGTGGAAGCGATCCTGGT | SWYLFQFHRLLQYARPKPGSPRPFFWMFV |
| | | CAGTTGAAACACGTGGTTGACGTCACAGACACGG | DNLVLNKEDLDVASRFLEMEPVTIPDVHG |
| | | TGAGGAAGGATGTTGAGGAGTGGGGTCCATTCGA | GSLQNAVRVWSNIPAIRSRHWALVSEEEL |
| | | TTTGGTCTATGGCGCAACCCCTCCGCTTGGACATA | SLLAQNKQSSKLAAKWPTKLVKNCFLPLR |
| | | CGTGCGATAGACCTCCGAGCTGGTATCTTTTCCAG | EYFKYFSTELTSSLGSGSGSGAPRNGTVHL |
| | | TTTCACAGGCTGCTTCAATACGCAAGACCGAAGCC | YLTKPLYTSAPSLQHLCRLTINKCTGAIWG |
| | | GGGTTCCCCTCGGCCCTTCTTCTGGATGTTTGTCGA | LPLPTRLKDYLEEYKFQVGSGSGSGSGGF |
| | | TAATCTCGTTTTGAATAAAGAGGATTTGGATGTAG | NVLMVHKRSHTGERPLQCEICGFTCRQKG |
| | | CATCCCGATTCCTTGAAATGGAACCGGTAACGATC | NLLRHIKLHTGEKPFKCHLCNYACQRRDA |
| | | CCCGATGTTCATGGTGGGAGCTTGCAGAACGCTGT | L (SEQ ID NO: 84) |
| | | CCGGGTGTGGAGTAACATACCTGCCATCCGGTCAA | |
| | | GGCACTGGGCTCTCGTAAGTGAGGAAGAACTCTC | |
| | | ACTGCTGGCGCAGAACAAGCAGTCTTCTAAATTGG | |
| | | CGGCAAAATGGCCCACCAAGCTTGTGAAAAATTG | |
| | | CTTTCTGCCCCTTCGAGAGTATTTTAAGTACTTTAG | |
| | | TACTGAACTCACATCATCACTGGGCAGTGGTAGTG | |
| | | GCtccggaGCTCCACGAAACGGCACGGTCCACCTTTA | |
| | | CTTGACGAAACCGCTTTATACCTCCGCACCGTCCC | |
| | | TCCAGCACCTGTGTCGGCTGACAATCAATAAGTGT | |
| | | ACAGGTGCGATATGGGGATTGCCGCTTCCGACCCG | |
| | | CCTTAAAGATTACCTCGAGGAATACAAATTTCAAG | |
| | | TCggcagtggctcgggctcggggtccggcggaTTCAATGTCTTAA | |
| | | TGGTTCATAAGCGAAGCCATACTGGTGAACGCCC | |
| | | ATTGCAGTGCGAAATATGCGGCTTTACCTGCCGCC | |
| | | AGAAAGGTAACCTCCTCCGCCACATTAAACTGCAC | |
| | | ACAGGGGAAAAACCTTTTAAGTGTCACCTCTGCAA | |
| | | CTATGCATGCCAAAGAAGAGATGCGCTC (SEQ ID | |
| | | NO: 83) | |
| pJL_087 | 913- | ATGTCTGGGTTCAATGTGCTGATGGTCCACAAGAG | MSGFNVLMVHKRSHTGERPLQCEICGFTC |
| | vhhGFP4- | AAGTCACACCGGCGAGCGGCCCCTGCAGTGCGAA | RQKGNLLRHIKLHTGEKPFKCHLCNYACQ |
| | SOCS2 | ATCTGTGGATTTACATGCCGCCAAAAAGGGAACTT | RRDALGSGSGSGSGGDQVQLVESGGALV |
| | | GCTTCGACACATAAAGCTGCATACCGGCGAGAAG | QPGGSLRLSCAASGFPVNRYSMRWYRQA |
| | | CCATTTAAGTGCCACTTGTGTAACTACGCTTGTCA | PGKEREWVAGMSSAGDRSSYEDSVKGRF |
| | | GAGGCGGGACGCATTGGGGAGCGGATCTGGATCA | TISRDDARNTVYLQMNSLKPEDTAVYYCN |
| | | GGAAGCGGGGGAGATCAAGTCCAACTGGTGGAGT | VNVGFEYWGQGTQVTVSSGSGSGSGAPR |
| | | CTGGTGGCGCTTTGGTGCAGCCAGGTGGCTCTCTG | NGTVHLYLTKPLYTSAPSLQHLCRLTINKC |
| | | CGTTTGTCCTGTGCCGCTTCTGGCTTCCCAGTGAA | TGAIWGLPLPTRLKDYLEEYKFQV (SEQ |
| | | CCGCTATTCCATGCGCTGGTATCGCCAGGCTCCAG | ID NO: 86) |
| | | GCAAAGAGCGTGAGTGGGTAGCCGGTATGTCCAG | |
| | | CGCGGGTGATCGTAGCTCCTATGAAGACTCCGTGA | |
| | | AGGGCCGTTTCACCATCAGCCGTGACGATGCCCGT | |
| | | AACACGGTGTATCTGCAAATGAACAGCTTGAAAC | |
| | | CTGAAGATACGGCCGTGTATTACTGTAATGTGAAC | |
| | | GTGGGCTTCGAGTATTGGGGCCAAGGCACCCAGG | |
| | | TCACCGTgTCCAGCGGCAGTGGTAGTGGCtccggaGC | |
| | | TCCACGAAACGGCACGGTCCACCTTTACTTGACGA | |
| | | AACCGCTTTATACCTCCGCACCGTCCCTCCAGCAC | |
| | | CTGTGTCGGCTGACAATCAATAAGTGTACAGGTGC | |
| | | GATATGGGGATTGCCGCTTCCGACCCGCCTTAAAG | |
| | | ATTACCTCGAGGAATACAAATTTCAAGTC (SEQ ID | |
| | | NO: 85) | |
| pJL_088 | vhhGFP4- | ATGggaggccttGATTACAAGGACGACGACGACAAGgc | MGGLDYKDDDDKASDQVQLVESGGALV |
| | SOCS2-913 | tagcGATCAAGTCCAACTGGTGGAGTCTGGTGGCGC | QPGGSLRLSCAASGFPVNRYSMRWYRQA |
| | | TTTGGTGCAGCCAGGTGGCTCTCTGCGTTTGTCCT | PGKEREWVAGMSSAGDRSSYEDSVKGRF |
| | | GTGCCGCTTCTGGCTTCCCAGTGAACCGCTATTCC | TISRDDARNTVYLQMNSLKPEDTAVYYCN |
| | | ATGCGCTGGTATCGCCAGGCTCCAGGCAAAGAGC | VNVGFEYWGQGTQVTVSSGSGSGSGSGG |
| | | GTGAGTGGGTAGCCGGTATGTCCAGCGCGGGTGA | FNVLMVHKRSHTGERPLQCEICGFTCRQK |
| | | TCGTAGCTCCTATGAAGACTCCGTGAAGGGCCGTT | GNLLRHIKLHTGEKPFKCHLCNYACQRRD |
| | | TCACCATCAGCCGTGACGATGCCCGTAACACGGTG | ALGSGSGSGAPRNGTVHLYLTKPLYTSAP |
| | | TATCTGCAAATGAACAGCTTGAAACCTGAAGATA | SLQHLCRLTINKCTGAIWGLPLPTRLKDYL |
| | | CGGCCGTGTATTACTGTAATGTGAACGTGGGCTTC | EEYKFQV (SEQ ID NO: 88) |
| | | GAGTATTGGGGCCAAGGCACCCAGGTCACCGTgTC | |
| | | CAGCggcagtggctcgggctcggggtccggcggaTTCAATGTCTT | |
| | | AATGGTTCATAAGCGAAGCCATACTGGTGAACGC | |
| | | CCATTGCAGTGCGAAATATGCGGCTTTACCTGCCG | |
| | | CCAGAAAGGTAACCTCCTCCGCCACATTAAACTGC | |
| | | ACACAGGGGAAAAACCTTTTAAGTGTCACCTCTGC | |
| | | AACTATGCATGCCAAAGAAGAGATGCGCTCGGCA | |
| | | GTGGTAGTGGCtccggaGCTCCACGAAACGGCACGG | |
| | | TCCACCTTTACTTGACGAAACCGCTTTATACCTCC | |
| | | GCACCGTCCCTCCAGCACCTGTGTCGGCTGACAAT | |
| | | CAATAAGTGTACAGGTGCGATATGGGGATTGCCG | |
| | | CTTCCGACCCGCCTTAAAGATTACCTCGAGGAATA | |
| | | CAAATTTCAAGTC (SEQ ID NO: 87) | |
| pJL_089 | vhhGFP4- | ATGggaggccttGATTACAAGGACGACGACGACAAGgc | MGGLDYKDDDDKASDQVQLVESGGALV |
| | 913-SOCS2 | tagcGATCAAGTCCAACTGGTGGAGTCTGGTGGCGC | QPGGSLRLSCAASGFPVNRYSMRWYRQA |
| | | TTTGGTGCAGCCAGGTGGCTCTCTGCGTTTGTCCT | PGKEREWVAGMSSAGDRSSYEDSVKGRF |
| | | GTGCCGCTTCTGGCTTCCCAGTGAACCGCTATTCC | TISRDDARNTVYLQMNSLKPEDTAVYYCN |
| | | ATGCGCTGGTATCGCCAGGCTCCAGGCAAAGAGC | VNVGFEYWGQGTQVTVSSGSGSGSGAPR |
| | | GTGAGTGGGTAGCCGGTATGTCCAGCGCGGGTGA | NGTVHLYLTKPLYTSAPSLQHLCRLTINKC |
| | | TCGTAGCTCCTATGAAGACTCCGTGAAGGGCCGTT | TGAIWGLPLPTRLKDYLEEYKFQVGSGSG |
| | | TCACCATCAGCCGTGACGATGCCCGTAACACGGTG | SGSGGFNVLMVHKRSHTGERPLQCEICGF |
| | | TATCTGCAAATGAACAGCTTGAAACCTGAAGATA | TCRQKGNLLRHIKLHTGEKPFKCHLCNYA |
| | | CGGCCGTGTATTACTGTAATGTGAACGTGGGCTTC | CQRRDAL (SEQ ID NO: 90) |
| | | GAGTATTGGGGCCAAGGCACCCAGGTCACCGTgTC | |
| | | CAGCGGCAGTGGTAGTGGCtccggaGCTCCACGAAA | |
| | | CGGCACGGTCCACCTTTACTTGACGAAACCGCTTT | |
| | | ATACCTCCGCACCGTCCCTCCAGCACCTGTGTCGG | |
| | | CTGACAATCAATAAGTGTACAGGTGCGATATGGG | |
| | | GATTGCCGCTTCCGACCCGCCTTAAAGATTACCTC | |
| | | GAGGAATACAAATTTCAAGTCggcagtggctcgggctcgggg | |
| | | tccggcggaTTCAATGTCTTAATGGTTCATAAGCGAAG | |
| | | CCATACTGGTGAACGCCCATTGCAGTGCGAAATAT | |
| | | GCGGCTTTACCTGCCGCCAGAAAGGTAACCTCCTC | |
| | | CGCCACATTAAACTGCACACAGGGGAAAAACCTT | |
| | | TTAAGTGTCACCTCTGCAACTATGCATGCCAAAGA | |
| | | AGAGATGCGCTC(SEQ ID NO: 89) | |
| pJL_090 | SV40NLS- | ATGgccccaaagaagaagcggaaggtcggtatccacggagtcccagc | MAPKKKRKVGIHGVPAAGSENPLEMFET |
| | DNMT3L- | agccggaagcGAGAATCCCTTGGAAATGTTTGAAACCGTG | VPVWRRQPVRVLSLFEDIKKELTSLGFLES |
| | SOCS2 | CCTGTTTGGAGAAGACAACCGGTGCGCGTACTTTC | GSDPGQLKHVVDVTDTVRKDVEEWGPFD |
| | | ATTGTTCGAAGACATTAAAAAGGAGCTCACCAGC | LVYGATPPLGHTCDRPPSWYLFQFHRLLQ |
| | | CTTGGGTTCCTCGAGAGTGGAAGCGATCCTGGTCA | YARPKPGSPRPFFWMFVDNLVLNKEDLD |
| | | GTTGAAACACGTGGTTGACGTCACAGACACGGTG | VASRFLEMEPVTIPDVHGGSLQNAVRVWS |
| | | AGGAAGGATGTTGAGGAGTGGGGTCCATTCGATT | NIPAIRSRHWALVSEEELSLLAQNKQSSKL |
| | | TGGTCTATGGCGCAACCCCTCCGCTTGGACATACG | AAKWPTKLVKNCFLPLREYFKYFSTELTS |
| | | TGCGATAGACCTCCGAGCTGGTATCTTTTCCAGTT | SLGSGSGSGAPRNGTVHLYLTKPLYTSAPS |
| | | TCACAGGCTGCTTCAATACGCAAGACCGAAGCCG | LQHLCRLTINKCTGAIWGLPLPTRLKDYLE |
| | | GGTTCCCCTCGGCCCTTCTTCTGGATGTTTGTCGAT | EYKFQV (SEQ ID NO: 92) |
| | | AATCTCGTTTTGAATAAAGAGGATTTGGATGTAGC | |
| | | ATCCCGATTCCTTGAAATGGAACCGGTAACGATCC | |
| | | CCGATGTTCATGGTGGGAGCTTGCAGAACGCTGTC | |
| | | CGGGTGTGGAGTAACATACCTGCCATCCGGTCAA | |
| | | GGCACTGGGCTCTCGTAAGTGAGGAAGAACTCTC | |
| | | ACTGCTGGCGCAGAACAAGCAGTCTTCTAAATTGG | |
| | | CGGCAAAATGGCCCACCAAGCTTGTGAAAAATTG | |
| | | CTTTCTGCCCCTTCGAGAGTATTTTAAGTACTTTAG | |
| | | TACTGAACTCACATCATCACTGGGCAGTGGTAGTG | |
| | | GCtccggaGCTCCACGAAACGGCACGGTCCACCTTTA | |
| | | CTTGACGAAACCGCTTTATACCTCCGCACCGTCCC | |
| | | TCCAGCACCTGTGTCGGCTGACAATCAATAAGTGT | |
| | | ACAGGTGCGATATGGGGATTGCCGCTTCCGACCCG | |
| | | CCTTAAAGATTACCTCGAGGAATACAAATTTCAAG | |
| | | TC(SEQ ID NO: 91) | |
| pJL_091 | DNMT3L- | ATGggaagcGAGAATCCCTTGGAAATGTTTGAAACC | MGSENPLEMFETVPVWRRQPVRVLSLFED |
| | SOCS2- | GTGCCTGTTTGGAGAAGACAACCGGTGCGCGTACT | IKKELTSLGFLESGSDPGQLKHVVDVTDT |
| | NPLNLS | TTCATTGTTCGAAGACATTAAAAAGGAGCTCACCA | VRKDVEEWGPFDLVYGATPPLGHTCDRPP |
| | | GCCTTGGGTTCCTCGAGAGTGGAAGCGATCCTGGT | SWYLFQFHRLLQYARPKPGSPRPFFWMFV |
| | | CAGTTGAAACACGTGGTTGACGTCACAGACACGG | DNLVLNKEDLDVASRFLEMEPVTIPDVHG |
| | | TGAGGAAGGATGTTGAGGAGTGGGGTCCATTCGA | GSLQNAVRVWSNIPAIRSRHWALVSEEEL |
| | | TTTGGTCTATGGCGCAACCCCTCCGCTTGGACATA | SLLAQNKQSSKLAAKWPTKLVKNCFLPLR |
| | | CGTGCGATAGACCTCCGAGCTGGTATCTTTTCCAG | EYFKYFSTELTSSLGSGSGSGAPRNGTVHL |
| | | TTTCACAGGCTGCTTCAATACGCAAGACCGAAGCC | YLTKPLYTSAPSLQHLCRLTINKCTGAIWG |
| | | GGGTTCCCCTCGGCCCTTCTTCTGGATGTTTGTCGA | LPLPTRLKDYLEEYKFQVGGGGSKRPAAT |
| | | TAATCTCGTTTTGAATAAAGAGGATTTGGATGTAG | KKAGQAKKKK (SEQ ID NO: 94) |
| | | CATCCCGATTCCTTGAAATGGAACCGGTAACGATC | |
| | | CCCGATGTTCATGGTGGGAGCTTGCAGAACGCTGT | |
| | | CCGGGTGTGGAGTAACATACCTGCCATCCGGTCAA | |
| | | GGCACTGGGCTCTCGTAAGTGAGGAAGAACTCTC | |
| | | ACTGCTGGCGCAGAACAAGCAGTCTTCTAAATTGG | |
| | | CGGCAAAATGGCCCACCAAGCTTGTGAAAAATTG | |
| | | CTTTCTGCCCCTTCGAGAGTATTTTAAGTACTTTAG | |
| | | TACTGAACTCACATCATCACTGGGCAGTGGTAGTG | |
| | | GCtccggaGCTCCACGAAACGGCACGGTCCACCTTTA | |
| | | CTTGACGAAACCGCTTTATACCTCCGCACCGTCCC | |
| | | TCCAGCACCTGTGTCGGCTGACAATCAATAAGTGT | |
| | | ACAGGTGCGATATGGGGATTGCCGCTTCCGACCCG | |
| | | CCTTAAAGATTACCTCGAGGAATACAAATTTCAAG | |
| | | TCGGCGGCGGCGGCTCCaagcgtcctgctgctactaagaaagctg | |
| | | gtcaagctaagaaaaagaaa (SEQ ID NO: 93) | |
| pJL_092 | c-mycNLS- | ATGcctgctgctaagagagtgaaactggatctcgagggaagcGAG | MPAAKRVKLDLEGSENPLEMFETVPVWR |
| | DNMT3L- | AATCCCTTGGAAATGTTTGAAACCGTGCCTGTTTGGAGA | RQPVRVLSLFEDIKKELTSLGFLESGSDPG |
| | SOCS2 | AGACAACCGGTGCGCGTACTTTCATTGTTCGAAGA | QLKHVVDVTDTVRKDVEEWGPFDLVYGA |
| | | CATTAAAAAGGAGCTCACCAGCCTTGGGTTCCTCG | TPPLGHTCDRPPSWYLFQFHRLLQYARPK |
| | | AGAGTGGAAGCGATCCTGGTCAGTTGAAACACGT | PGSPRPFFWMFVDNLVLNKEDLDVASRFL |
| | | GGTTGACGTCACAGACACGGTGAGGAAGGATGTT | EMEPVTIPDVHGGSLQNAVRVWSNIPAIRS |
| | | GAGGAGTGGGGTCCATTCGATTTGGTCTATGGCGC | RHWALVSEEELSLLAQNKQSSKLAAKWP |
| | | AACCCCTCCGCTTGGACATACGTGCGATAGACCTC | TKLVKNCFLPLREYFKYFSTELTSSLGSGS |
| | | CGAGCTGGTATCTTTTCCAGTTTCACAGGCTGCTT | GSGAPRNGTVHLYLTKPLYTSAPSLQHLC |
| | | CAATACGCAAGACCGAAGCCGGGTTCCCCTCGGC | RLTINKCTGAIWGLPLPTRLKDYLEEYKFQ |
| | | CCTTCTTCTGGATGTTTGTCGATAATCTCGTTTTGA | V (SEQ ID NO: 163) |
| | | ATAAAGAGGATTTGGATGTAGCATCCCGATTCCTT | |
| | | GAAATGGAACCGGTAACGATCCCCGATGTTCATG | |
| | | GTGGGAGCTTGCAGAACGCTGTCCGGGTGTGGAG | |
| | | TAACATACCTGCCATCCGGTCAAGGCACTGGGCTC | |
| | | TCGTAAGTGAGGAAGAACTCTCACTGCTGGCGCA | |
| | | GAACAAGCAGTCTTCTAAATTGGCGGCAAAATGG | |
| | | CCCACCAAGCTTGTGAAAAATTGCTTTCTGCCCCT | |
| | | TCGAGAGTATTTTAAGTACTTTAGTACTGAACTCA | |
| | | CATCATCACTGGGCAGTGGTAGTGGCtccggaGCTCC | |
| | | ACGAAACGGCACGGTCCACCTTTACTTGACGAAA | |
| | | CCGCTTTATACCTCCGCACCGTCCCTCCAGCACCT | |
| | | GTGTCGGCTGACAATCAATAAGTGTACAGGTGCG | |
| | | ATATGGGGATTGCCGCTTCCGACCCGCCTTAAAGA | |
| | | TTACCTCGAGGAATACAAATTTCAAGTC (SEQ ID | |
| | | NO: 162) | |
| pJL_093 | DNMT3L- | AAGGGCGGCGGCGGCTCCGGCGGCGGCGGCTCCG | KGGGGSGGGGSENPLEMFETVPVWRRQP |
| | SOCS2 | AGAATCCCTTGGAAATGTTTGAAACCGTGCCTGTT | VRVLSLFEDIKKELTSLGFLESGSDPGQLK |
| | | TGGAGAAGACAACCGGTGCGCGTACTTTCATTGTT | HVVDVTDTVRKDVEEWGPFDLVYGATPP |
| | | CGAAGACATTAAAAAGGAGCTCACCAGCCTTGGG | LGHTCDRPPSWYLFQFHRLLQYARPKPGS |
| | | TTCCTCGAGAGTGGAAGCGATCCTGGTCAGTTGAA | PRPFFWMFVDNLVLNKEDLDVASRFLEM |
| | | ACACGTGGTTGACGTCACAGACACGGTGAGGAAG | EPVTIPDVHGGSLQNAVRVWSNIPAIRSRH |
| | | GATGTTGAGGAGTGGGGTCCATTCGATTTGGTCTA | WALVSEEELSLLAQNKQSSKLAAKWPTK |
| | | TGGCGCAACCCCTCCGCTTGGACATACGTGCGATA | LVKNCFLPLREYFKYFSTELTSSLGSGSGS |
| | | GACCTCCGAGCTGGTATCTTTTCCAGTTTCACAGG | GAPRNGTVHLYLTKPLYTSAPSLQHLCRL |
| | | CTGCTTCAATACGCAAGACCGAAGCCGGGTTCCCC | TINKCTGAIWGLPLPTRLKDYLEEYKFQV* |
| | | TCGGCCCTTCTTCTGGATGTTTGTCGATAATCTCGT | (SEQ ID NO: 96) |
| | | TTTGAATAAAGAGGATTTGGATGTAGCATCCCGAT | |
| | | TCCTTGAAATGGAACCGGTAACGATCCCCGATGTT | |
| | | CATGGTGGGAGCTTGCAGAACGCTGTCCGGGTGT | |
| | | GGAGTAACATACCTGCCATCCGGTCAAGGCACTG | |
| | | GGCTCTCGTAAGTGAGGAAGAACTCTCACTGCTGG | |
| | | CGCAGAACAAGCAGTCTTCTAAATTGGCGGCAAA | |
| | | ATGGCCCACCAAGCTTGTGAAAAATTGCTTTCTGC | |
| | | CCCTTCGAGAGTATTTTAAGTACTTTAGTACTGAA | |
| | | CTCACATCATCACTGGGCAGTGGTAGTGGCtccggaG | |
| | | CTCCACGAAACGGCACGGTCCACCTTTACTTGACG | |
| | | AAACCGCTTTATACCTCCGCACCGTCCCTCCAGCA | |
| | | CCTGTGTCGGCTGACAATCAATAAGTGTACAGGTG | |
| | | CGATATGGGGATTGCCGCTTCCGACCCGCCTTAAA | |
| | | GATTACCTCGAGGAATACAAATTTCAAGTCTAA | |
| | | (SEQ ID NO: 95) | |
| pJL_094 | vhhGFP4- | ATGggaggccttGATTACAAGGACGACGACGACAAGgc | MGGLDYKDDDDKASDQVQLVESGGALV |
| | SOCS2 | tagcGATCAAGTCCAACTGGTGGAGTCTGGTGGCGC | QPGGSLRLSCAASGFPVNRYSMRWYRQA |
| | | TTTGGTGCAGCCAGGTGGCTCTCTGCGTTTGTCCT | PGKEREWVAGMSSAGDRSSYEDSVKGRF |
| | | GTGCCGCTTCTGGCTTCCCAGTGAACCGCTATTCC | TISRDDARNTVYLQMNSLKPEDTAVYYCN |
| | | ATGCGCTGGTATCGCCAGGCTCCAGGCAAAGAGC | VNVGFEYWGQGTQVTVSSGSGSGSGAPR |
| | | GTGAGTGGGTAGCCGGTATGTCCAGCGCGGGTGA | NGTVHLYLTKPLYTSAPSLQHLCRLTINKC |
| | | TCGTAGCTCCTATGAAGACTCCGTGAAGGGCCGTT | TGAIWGLPLPTRLKDYLEEYKFQV* (SEQ |
| | | TCACCATCAGCCGTGACGATGCCCGTAACACGGTG | ID NO: 98) |
| | | TATCTGCAAATGAACAGCTTGAAACCTGAAGATA | |
| | | CGGCCGTGTATTACTGTAATGTGAACGTGGGCTTC | |
| | | GAGTATTGGGGCCAAGGCACCCAGGTCACCGTgTC | |
| | | CAGCGGCAGTGGTAGTGGCtccggaGCTCCACGAAA | |
| | | CGGCACGGTCCACCTTTACTTGACGAAACCGCTTT | |
| | | ATACCTCCGCACCGTCCCTCCAGCACCTGTGTCGG | |
| | | CTGACAATCAATAAGTGTACAGGTGCGATATGGG | |
| | | GATTGCCGCTTCCGACCCGCCTTAAAGATTACCTC | |
| | | GAGGAATACAAATTTCAAGTCTAA (SEQ ID NO: 97) | |
| pJL_095 | vhhGFP4- | ATGAGCGAGCTGATTAAGGAGAACATGCACATGA | MSELIKENMHMKLYMEGTVDNHHFKCTS |
| | SOCS2 | AGCTGTACATGGAGGGCACCGTGGACAACCATCA | EGEGKPYEGTQTMRIKVVEGGPLPFAFDIL |
| | | CTTCAAGTGCACATCCGAGGGCGAAGGCAAGCCC | ATSFLYGSKTFINHTQGIPDFFKQSFPEGFT |
| | | TACGAGGGCACCCAGACCATGAGAATCAAGGTGG | WERVTTYEDGGVLTATQDTSLQDGCLIYN |
| | | TCGAGGGCGGCCCTCTCCCCTTCGCCTTCGACATC | VKIRGVNFTSNGPVMQKKTLGWEAFTETL |
| | | CTGGCTACTAGCTTCCTCTACGGCAGCAAGACCTT | YPADGGLEGRNDMALKLVGGSHLIANIKT |
| | | CATCAACCACACCCAGGGCATCCCCGACTTCTTCA | TYRSKKPAKNLKMPGVYYVDYRLERIKE |
| | | AGCAGTCCTTCCCTGAGGGCTTCACATGGGAGAG | ANNETYVEQHEVAVARYCDLPSKLGHKL |
| | | AGTCACCACATACGAAGACGGGGGCGTGCTGACC | NGSGATNFSLLKQAGDVEENPGPMGGLD |
| | | GCTACCCAGGACACCAGCCTCCAGGACGGCTGCC | YKDDDDKASDQVQLVESGGALVQPGGSL |
| | | TCATCTACAACGTCAAGATCAGAGGGGTGAACTTC | RLSCAASGFPVNRYSMRWYRQAPGKERE |
| | | ACATCCAACGGCCCTGTGATGCAGAAGAAAACAC | WVAGMSSAGDRSSYEDSVKGRFTISRDDA |
| | | TCGGCTGGGAGGCCTTCACCGAGACGCTGTACCCC | RNTVYLQMNSLKPEDTAVYYCNVNVGFE |
| | | GCTGACGGCGGCCTGGAAGGCAGAAACGACATGG | YWGQGTQVTVSSGSGSGSGAPRNGTVHL |
| | | CCCTGAAGCTCGTGGGCGGGAGCCATCTGATCGC | YLTKPLYTSAPSLQHLCRLTINKCTGAIWG |
| | | AAACATCAAGACCACATATAGATCCAAGAAACCC | LPLPTRLKDYLEEYKFQV* (SEQ ID NO: |
| | | GCTAAGAACCTCAAGATGCCTGGCGTCTACTATGT | 100) |
| | | GGACTACAGACTGGAAAGAATCAAGGAGGCCAAC | |
| | | AACGAGACCTACGTCGAGCAGCACGAGGTGGCAG | |
| | | TGGCCAGATACTGCGACCTCCCTAGCAAACTGGG | |
| | | GCACAAGCTTAATGGTTCAGGTGCAACGAACTTCT | |
| | | CATTGTTGAAGCAAGCCGGTGATGTTGAGGAAAA | |
| | | TCCGGGTCCTATGggaggccttGATTACAAGGACGACG | |
| | | ACGACAAGgctagcGATCAAGTCCAACTGGTGGAGT | |
| | | CTGGTGGCGCTTTGGTGCAGCCAGGTGGCTCTCTG | |
| | | CGTTTGTCCTGTGCCGCTTCTGGCTTCCCAGTGAA | |
| | | CCGCTATTCCATGCGCTGGTATCGCCAGGCTCCAG | |
| | | GCAAAGAGCGTGAGTGGGTAGCCGGTATGTCCAG | |
| | | CGCGGGTGATCGTAGCTCCTATGAAGACTCCGTGA | |
| | | AGGGCCGTTTCACCATCAGCCGTGACGATGCCCGT | |
| | | AACACGGTGTATCTGCAAATGAACAGCTTGAAAC | |
| | | CTGAAGATACGGCCGTGTATTACTGTAATGTGAAC | |
| | | GTGGGCTTCGAGTATTGGGGCCAAGGCACCCAGG | |
| | | TCACCGTgTCCAGCGGCAGTGGTAGTGGCtccggaGC | |
| | | TCCACGAAACGGCACGGTCCACCTTTACTTGACGA | |
| | | AACCGCTTTATACCTCCGCACCGTCCCTCCAGCAC | |
| | | CTGTGTCGGCTGACAATCAATAAGTGTACAGGTGC | |
| | | GATATGGGGATTGCCGCTTCCGACCCGCCTTAAAG | |
| | | ATTACCTCGAGGAATACAAATTTCAAGTCTAA | |
| | | (SEQ ID NO: 99) | |
| pJL_097 | SARA- | ATGAGCGCGTCATCCCAGAGCCCTAACCCAAATA | MSASSQSPNPNNPAEYCSTIPPLQQAQASG |
| | SOCS2 | ATCCCGCAGAATACTGCAGCACAATACCCCCTTTG | ALSSPPPTVMVPVGVLKHPGAEVAQPRGS |
| | | CAGCAGGCACAAGCTAGTGGCGCTTTGAGCAGCC | GSGSGAPRNGTVHLYLTKPLYTSAPSLQH |
| | | CACCGCCCACGGTCATGGTTCCTGTGGGAGTCCTT | LCRLTINKCTGAIWGLPLPTRLKDYLEEYK |
| | | AAACATCCAGGGGCTGAAGTAGCACAGCCCCGGG | FQV (SEQ ID NO: 102) |
| | | GCAGTGGTAGTGGCtccggaGCTCCACGAAACGGCA | |
| | | CGGTCCACCTTTACTTGACGAAACCGCTTTATACC | |
| | | TCCGCACCGTCCCTCCAGCACCTGTGTCGGCTGAC | |
| | | AATCAATAAGTGTACAGGTGCGATATGGGGATTG | |
| | | CCGCTTCCGACCCGCCTTAAAGATTACCTCGAGGA | |
| | | ATACAAATTTCAAGTC (SEQ ID NO: 101) | |
| pJL_098 | SARA-VPX | ATGggaggccttGATTACAAGGACGACGACGACAAGgc | MGGLDYKDDDDKASMSASSQSPNPNNPA |
| | | tagcATGAGCGCGTCATCCCAGAGCCCTAACCCAAA | EYCSTIPPLQQAQASGALSSPPPTVMVPVG |
| | | TAATCCCGCAGAATACTGCAGCACAATACCCCCTT | VLKHPGAEVAQPRGSGSGSGADPRERVPP |
| | | TGCAGCAGGCACAAGCTAGTGGCGCTTTGAGCAG | GNSGEETIGEAFEWLDRTIEALNREAVNH |
| | | CCCACCGCCCACGGTCATGGTTCCTGTGGGAGTCC | LPRELIFQVWQRSWAYWHDEQGMSTSYT |
| | | TTAAACATCCAGGGGCTGAAGTAGCACAGCCCCG | KYRYLCIMQKAVYIHFKKGCTCLGRGHGP |
| | | GGGCAGTGGTAGTGGCtccggaGCAGACCCGAGGGA | GGWRPGPPPPPPPGLV(SEQ ID NO: 104) |
| | | AAGAGTTCCACCAGGTAACAGTGGCGAAGAAACG | |
| | | ATCGGCGAAGCGTTCGAGTGGCTTGATCGGACTAT | |
| | | TGAGGCTCTTAATCGGGAGGCTGTGAACCACCTGC | |
| | | CCCGGGAACTTATTTTCCAGGTCTGGCAGAGAAGT | |
| | | TGGGCCTACTGGCACGATGAGCAAGGCATGTCCA | |
| | | CATCCTATACAAAGTATCGGTATCTTTGTATAATG | |
| | | CAGAAGGCTGTATATATCCACTTTAAGAAGGGAT | |
| | | GTACTTGTCTGGGTAGAGGTCATGGACCAGGAGG | |
| | | GTGGAGACCAGGCCCTCCACCTCCTCCACCTCCAG | |
| | | GTCTTGTG(SEQ ID NO: 103) | |
| pJL_099 | 913iK0- | ATGggaggccttGATTACAAGGACGACGACGACAAGgc | MGGLDYKDDDDKASFNVLMVHRRSHTG |
| | SARA(K0) | tagcTTCAATGTCTTAATGGTTCATcggCGAAGCCATA | ERPLQCEICGFTCRQRGNLLRHIRLHTGER |
| | | CTGGTGAACGCCCATTGCAGTGCGAAATATGCGG | PFRCHLCNYACQRRDALSGSGSGSGMSAS |
| | | CTTTACCTGCCGCCAGcgcGGTAACCTCCTCCGCCA | SQSPNPNNPAEYCSTIPPLQQAQASGALSS |
| | | CATTcgtCTGCACACAGGGGAAcggCCTTTTcggTGTC | PPPTVMVPVGVLRHPGAEVAQPR (SEQ ID |
| | | ACCTCTGCAACTATGCATGCCAAAGAAGAGATGC | NO: 106) |
| | | GCTCAGCGGGTCAGGTAGTGGCtccggaATGAGCGC | |
| | | GTCATCCCAGAGCCCTAACCCAAATAATCCCGCAG | |
| | | AATACTGCAGCACAATACCCCCTTTGCAGCAGGCA | |
| | | CAAGCTAGTGGCGCTTTGAGCAGCCCACCGCCCAC | |
| | | GGTCATGGTTCCTGTGGGAGTCCTTAgACATCCAG | |
| | | GGGCTGAAGTAGCACAGCCCCGG (SEQ ID NO: 105) | |
| pJL_100 | SMAD2 | AAGGGCGGCGGCGGCTCCGGCGGCGGCGGCTCCA | KGGGGSGGGGSTLSPVNHSLDLQPVTYSE |
| | | CGCTCAGTCCCGTTAACCACAGTCTTGACTTGCAG | PAFWCSIAYYELNQRVGETFHASQPSLTV |
| | | CCAGTCACCTACAGTGAGCCTGCCTTCTGGTGCTC | DGFTDPSNSERFCLGLLSNVNRNATVEMT |
| | | AATAGCCTACTATGAGTTGAATCAAAGAGTCGGT | RRHIGRGVRLYYIGGEVFAECLSDSAIFVQ |
| | | GAAACCTTCCATGCTTCACAACCAAGCCTGACTGT | SPNCNQRYGWHPATVCKIPPGCNLKIFNN |
| | | CGACGGATTCACTGACCCAAGTAATAGCGAAAGG | QEFAALLAQSVNQGFEAVYQLTRMCTIR |
| | | TTCTGCCTTGGGTTGCTGTCCAATGTAAACCGGAA | MSFVKGWGAEYRRQTVTSTPCWIELHLN |
| | | CGCCACAGTCGAGATGACAAGAAGGCATATCGGT | GPLQWLDKVLTQMGSPSVRCSSMS* (SEQ |
| | | CGAGGAGTGAGACTTTACTACATTGGGGGTGAAG | ID NO: 108) |
| | | TGTTCGCTGAGTGCTTGAGTGACTCTGCGATATTC | |
| | | GTACAGTCACCTAACTGTAACCAACGGTACGGAT | |
| | | GGCACCCTGCCACAGTTTGTAAAATACCCCCTGGA | |
| | | TGCAATCTCAAGATTTTCAACAATCAGGAGTTCGC | |
| | | TGCGCTGTTGGCCCAATCTGTCAACCAAGGTTTCG | |
| | | AAGCGGTCTACCAACTGACGCGAATGTGTACGAT | |
| | | ACGCATGTCTTTTGTGAAGGGGTGGGGTGCCGAGT | |
| | | ACAGGCGACAAACGGTCACTAGCACACCCTGCTG | |
| | | GATCGAGCTTCATTTGAACGGACCCCTTCAGTGGC | |
| | | TGGATAAGGTACTGACGCAGATGGGGTCACCAAG | |
| | | CGTGCGCTGTAGTTCTATGTCTTAA (SEQ ID NO: 107) | |
| pJL_101 | SMAD3 | AAGGGCGGCGGCGGCTCCGGCGGCGGCGGCTCCG | KGGGGSGGGGSAHNNLDLQPVTYCEPAF |
| | | CTCATAACAACTTGGATCTGCAGCCGGTAACCTAT | WCSISYYELNQRVGETFHASQPSMTVDGF |
| | | TGTGAGCCTGCGTTTTGGTGTTCTATTAGCTACTAT | TDPSNSERFOLGLLSNVNRNAAVELTRRHI |
| | | GAACTTAATCAAAGGGTGGGGGAAACGTTCCACG | GRGVRLYYIGGEVFAECLSDSAIFVQSPNC |
| | | CCTCTCAGCCCTCTATGACTGTGGACGGCTTTACC | NQRYGWHPATVCKIPPGCNLKIFNNQEFA |
| | | GACCCATCAAACTCCGAGAGATTCTGCCTCGGGTT | ALLAQSVNQGFEAVYQLTRMCTIRMSFVK |
| | | GCTTAGCAACGTGAATAGAAATGCGGCAGTCGAG | GWGAEYRRQTVTSTPCWIELHLNGPLQW |
| | | CTCACCCGACGGCATATAGGTAGAGGAGTACGCT | LDKVLTQMGSPSIRCSSVS* (SEQ ID NO: |
| | | TGTACTACATTGGAGGAGAAGTATTTGCGGAATGT | 110) |
| | | CTCTCCGACTCCGCTATCTTTGTACAATCACCTAA | |
| | | CTGCAACCAAAGATACGGGTGGCACCCGGCCACA | |
| | | GTATGTAAGATACCCCCCGGGTGTAACCTCAAGAT | |
| | | TTTCAATAATCAGGAGTTTGCCGCCCTTCTTGCCC | |
| | | AAAGTGTTAATCAAGGCTTTGAAGCGGTGTATCAG | |
| | | TTGACCAGAATGTGTACGATACGAATGTCATTTGT | |
| | | TAAAGGGTGGGGCGCAGAATACCGGCGACAAACT | |
| | | GTGACTTCAACGCCGTGTTGGATTGAGCTGCACCT | |
| | | TAACGGTCCCCTCCAGTGGCTGGACAAAGTACTTA | |
| | | CGCAGATGGGGAGCCCCAGCATTAGGTGCTCTAG | |
| | | CGTAAGTTAA (SEQ ID NO: 109) | |
| pJL_103 | SARA- | ATGAGCGCGTCATCCCAGAGCCCTAACCCAAATA | MSASSQSPNPNNPAEYCSTIPPLQQAQASG |
| | SOCS2 P2A | ATCCCGCAGAATACTGCAGCACAATACCCCCTTTG | ALSSPPPTVMVPVGVLKHPGAEVAQPRGS |
| | FMC63 | CAGCAGGCACAAGCTAGTGGCGCTTTGAGCAGCC | GSGSGAPRNGTVHLYLTKPLYTSAPSLQH |
| | | CACCGCCCACGGTCATGGTTCCTGTGGGAGTCCTT | LCRLTINKCTGAIWGLPLPTRLKDYLEEYK |
| | | AAACATCCAGGGGCTGAAGTAGCACAGCCCCGGG | FQVGSGATNFSLLKQAGDVEENPGPMALP |
| | | GCAGTGGTAGTGGCtccggaGCTCCACGAAACGGCA | VTALLLPLALLLHAARPEQKLISEEDLDIQ |
| | | CGGTCCACCTTTACTTGACGAAACCGCTTTATACC | MTQTTSSLSASLGDRVTISCRASQDISKYL |
| | | TCCGCACCGTCCCTCCAGCACCTGTGTCGGCTGAC | NWYQQKPDGTVKLLIYHTSRLHSGVPSRF |
| | | AATCAATAAGTGTACAGGTGCGATATGGGGATTG | SGSGSGTDYSLTISNLEQEDIATYFCQQGN |
| | | CCGCTTCCGACCCGCCTTAAAGATTACCTCGAGGA | TLPYTFGGGTKLEITGSTSGSGKPGSGEGS |
| | | ATACAAATTTCAAGTCGGTTCAGGTGCAACGAACT | TKGEVKLQESGPGLVAPSQSLSVTCTVSG |
| | | TCTCATTGTTGAAGCAAGCCGGTGATGTTGAGGAA | VSLPDYGVSWIRQPPRKGLEWLGVIWGSE |
| | | AATCCGGGTCCTATGGCTTTGCCTGTTACTGCGCT | TTYYNSALKSRLTIIKDNSKSQVFLKMNSL |
| | | TCTTTTGCCTTTGGCATTGTTGCTTCACGCCGCCAG | QTDDTAIYYCAKHYYYGGSYAMDYWGQ |
| | | GCCCGAGCAGAAGCTGATCAGCGAGGAGGACCTG | GTSVTVSS * (SEQ ID NO: 112) |
| | | GACATACAGATGACGCAAACAACTTCCAGTCTTA | |
| | | GCGCTAGCCTGGGGGATCGAGTCACCATATCTTGC | |
| | | AGGGCGTCcCAAGACATTAGCAAGTATCTCAATTG | |
| | | GTATCAACAGAAACCTGATGGAACAGTTAAACTT | |
| | | CTGATTTACCACACGAGTCGCCTGCACTCCGGTGT | |
| | | GCCCTCCAGATTCTCCGGCTCAGGAAGTGGAACCG | |
| | | ACTACTCTCTCACCATCTCCAACCTCGAACAAGAA | |
| | | GACATAGCTACATACTTTTGCCAACAAGGTAATAC | |
| | | CCTCCCCTATACCTTCGGTGGAGGCACTAAGCTGG | |
| | | AGATCACAGGGAGCACGTCCGGGTCTGGCAAACC | |
| | | GGGGAGTGGTGAGGGGTCTACGAAGGGAGAAGTC | |
| | | AAGCTTCAGGAGTCAGGACCCGGTCTTGTAGCTCC | |
| | | CAGCCAAAGCCTGTCAGTTACATGCACGGTTTCCG | |
| | | GTGTGTCTTTGCCAGATTATGGCGTATCTTGGATT | |
| | | CGCCAACCGCCTAGAAAGGGACTTGAGTGGTTGG | |
| | | GTGTCATTTGGGGATCAGAAACAACTTACTATAAC | |
| | | AGTGCTCTTAAGTCCAGGTTGACTATAATCAAGGA | |
| | | CAATAGTAAGTCCCAAGTTTTTCTGAAAATGAATT | |
| | | CCCTGCAGACAGATGACACCGCTATCTACTACTGT | |
| | | GCCAAGCACTACTATTATGGGGGCTCTTATGCTAT | |
| | | GGACTATTGGGGTCAGGGGACATCAGTTACTGTTT | |
| | | CCAGC * (SEQ ID NO: 111) | |
| pJL_104 | 913iK0- | ATGggaggccttGATTACAAGGACGACGACGACAAGgc | MGGLDYKDDDDKASFNVLMVHRRSHTG |
| | SARA(K0) | tagcTTCAATGTCTTAATGGTTCATcggCGAAGCCATA | ERPLQCEICGFTCRQRGNLLRHIRLHTGER |
| | P2A FMC63 | CTGGTGAACGCCCATTGCAGTGCGAAATATGCGG | PFRCHLCNYACQRRDALSGSGSGSGMSAS |
| | | CTTTACCTGCCGCCAGcgcGGTAACCTCCTCCGCCA | SQSPNPNNPAEYCSTIPPLQQAQASGALSS |
| | | CATTcgtCTGCACACAGGGGAAcggCCTTTTcggTGTC | PPPTVMVPVGVLRHPGAEVAQPRGSGATN |
| | | ACCTCTGCAACTATGCATGCCAAAGAAGAGATGC | FSLLKQAGDVEENPGPMALPVTALLLPLA |
| | | GCTCAGCGGGTCAGGTAGTGGCtccggaATGAGCGC | LLLHAARPEQKLISEEDLDIQMTQTTSSLS |
| | | GTCATCCCAGAGCCCTAACCCAAATAATCCCGCAG | ASLGDRVTISCRASQDISKYLNWYQQKPD |
| | | AATACTGCAGCACAATACCCCCTTTGCAGCAGGCA | GTVKLLIYHTSRLHSGVPSRFSGSGSGTDY |
| | | CAAGCTAGTGGCGCTTTGAGCAGCCCACCGCCCAC | SLTISNLEQEDIATYFCQQGNTLPYTFGGG |
| | | GGTCATGGTTCCTGTGGGAGTCCTTAgACATCCAG | TKLEITGSTSGSGKPGSGEGSTKGEVKLQE |
| | | GGGCTGAAGTAGCACAGCCCCGGGGTTCAGGTGC | SGPGLVAPSQSLSVTCTVSGVSLPDYGVS |
| | | AACGAACTTCTCATTGTTGAAGCAAGCCGGTGATG | WIRQPPRKGLEWLGVIWGSETTYYNSALK |
| | | TTGAGGAAAATCCGGGTCCTATGGCTTTGCCTGTT | SRLTIIKDNSKSQVFLKMNSLQTDDTAIYY |
| | | ACTGCGCTTCTTTTGCCTTTGGCATTGTTGCTTCAC | CAKHYYYGGSYAMDYWGQGTSVTVSS * |
| | | GCCGCCAGGCCCGAGCAGAAGCTGATCAGCGAGG | (SEQ ID NO: 114) |
| | | AGGACCTGGACATACAGATGACGCAAACAACTTC | |
| | | CAGTCTTAGCGCTAGCCTGGGGGATCGAGTCACCA | |
| | | TATCTTGCAGGGCGTCcCAAGACATTAGCAAGTAT | |
| | | CTCAATTGGTATCAACAGAAACCTGATGGAACAG | |
| | | TTAAACTTCTGATTTACCACACGAGTCGCCTGCAC | |
| | | TCCGGTGTGCCCTCCAGATTCTCCGGCTCAGGAAG | |
| | | TGGAACCGACTACTCTCTCACCATCTCCAACCTCG | |
| | | AACAAGAAGACATAGCTACATACTTTTGCCAACA | |
| | | AGGTAATACCCTCCCCTATACCTTCGGTGGAGGCA | |
| | | CTAAGCTGGAGATCACAGGGAGCACGTCCGGGTC | |
| | | TGGCAAACCGGGGAGTGGTGAGGGGTCTACGAAG | |
| | | GGAGAAGTCAAGCTTCAGGAGTCAGGACCCGGTC | |
| | | TTGTAGCTCCCAGCCAAAGCCTGTCAGTTACATGC | |
| | | ACGGTTTCCGGTGTGTCTTTGCCAGATTATGGCGT | |
| | | ATCTTGGATTCGCCAACCGCCTAGAAAGGGACTTG | |
| | | AGTGGTTGGGTGTCATTTGGGGATCAGAAACAACT | |
| | | TACTATAACAGTGCTCTTAAGTCCAGGTTGACTAT | |
| | | AATCAAGGACAATAGTAAGTCCCAAGTTTTTCTGA | |
| | | AAATGAATTCCCTGCAGACAGATGACACCGCTATC | |
| | | TACTACTGTGCCAAGCACTACTATTATGGGGGCTC | |
| | | TTATGCTATGGACTATTGGGGTCAGGGGACATCAG | |
| | | TTACTGTTTCCAGC * (SEQ ID NO: 113) | |
| pJL_105 | CAR19 P2A | TACTGTAAGCGCGGTCGGAAGAAGCTGCTGTACA | YCKRGRKKLLYIFKQPFMRPVQTTQEEDG |
| | SARA- | TCTTTAAGCAACCCTTCATGAGGCCTGTGCAGACT | CSCRFPEEEEGGCELRVKFSRSADAPAYQ |
| | SOCS2 | ACTCAAGAGGAGGACGGCTGTTCATGCCGGTTCCC | QGQNQLYNELNLGRREEYDVLDKRRGRD |
| | | AGAGGAGGAGGAAGGCGGCTGCGAACTGCGCGTG | PEMGGKPRRKNPQEGLYNELQKDKMAEA |
| | | AAATTCAGCCGCAGCGCAGATGCTCCAGCCTACC | YSEIGMKGERRRGKGHDGLYQGLSTATK |
| | | AACAGGGGCAGAACCAGCTCTACAACGAACTCAA | DTYDALHMQALPPRGSGATNFSLLKQAG |
| | | TCTTGGTCGGAGAGAGGAGTACGACGTGCTGGAC | DVEENPGPMSASSQSPNPNNPAEYCSTIPP |
| | | AAGCGGAGAGGACGGGACCCAGAAATGGGCGGG | LQQAQASGALSSPPPTVMVPVGVLKHPGA |
| | | AAGCCGCGCAGAAAGAATCCCCAAGAGGGCCTGT | EVAQPRGSGSGSGAPRNGTVHLYLTKPLY |
| | | ACAACGAGCTCCAAAAGGATAAGATGGCAGAAGC | TSAPSLQHLCRLTINKCTGAIWGLPLPTRL |
| | | CTATAGCGAGATTGGTATGAAAGGGGAACGCAGA | KDYLEEYKFQVSGGGGEGRGSLLTCGDV |
| | | AGAGGCAAAGGCCACGACGGACTGTACCAGGGAC | EENPGPRM * (SEQ ID NO: 116) |
| | | TCAGCACCGCCACCAAGGACACCTATGACGCTCTT | |
| | | CACATGCAGGCCCTGCCGCCTCGGGGTTCAGGTGC | |
| | | AACGAACTTCTCATTGTTGAAGCAAGCCGGTGATG | |
| | | TTGAGGAAAATCCGGGTCCTATGAGCGCGTCATCC | |
| | | CAGAGCCCTAACCCAAATAATCCCGCAGAATACT | |
| | | GCAGCACAATACCCCCTTTGCAGCAGGCACAAGC | |
| | | TAGTGGCGCTTTGAGCAGCCCACCGCCCACGGTCA | |
| | | TGGTTCCTGTGGGAGTCCTTAAACATCCAGGGGCT | |
| | | GAAGTAGCACAGCCCCGGGGCAGTGGTAGTGGCtc | |
| | | cggaGCTCCACGAAACGGCACGGTCCACCTTTACTT | |
| | | GACGAAACCGCTTTATACCTCCGCACCGTCCCTCC | |
| | | AGCACCTGTGTCGGCTGACAATCAATAAGTGTACA | |
| | | GGTGCGATATGGGGATTGCCGCTTCCGACCCGCCT | |
| | | TAAAGATTACCTCGAGGAATACAAATTTCAAGTCT | |
| | | CCGGAGGCGGCGGAGAGGGCAGAGGAAGTCTTCT | |
| | | AACATGCGGTGACGTGGAGGAGAATCCCGGCCCT | |
| | | AGGATG * (SEQ ID NO: 115) | |
| pJL_106 | CAR19 P2A | TACTGTAAGCGCGGTCGGAAGAAGCTGCTGTACA | YCKRGRKKLLYIFKQPFMRPVQTTQEEDG |
| | 913iK0- | TCTTTAAGCAACCCTTCATGAGGCCTGTGCAGACT | CSCRFPEEEEGGCELRVKFSRSADAPAYQ |
| | SARA(K0) | ACTCAAGAGGAGGACGGCTGTTCATGCCGGTTCCC | QGQNQLYNELNLGRREEYDVLDKRRGRD |
| | | AGAGGAGGAGGAAGGCGGCTGCGAACTGCGCGTG | PEMGGKPRRKNPQEGLYNELQKDKMAEA |
| | | AAATTCAGCCGCAGCGCAGATGCTCCAGCCTACC | YSEIGMKGERRRGKGHDGLYQGLSTATK |
| | | AACAGGGGCAGAACCAGCTCTACAACGAACTCAA | DTYDALHMQALPPRGSGATNFSLLKQAG |
| | | TCTTGGTCGGAGAGAGGAGTACGACGTGCTGGAC | DVEENPGPMGGLDYKDDDDKASFNVLM |
| | | AAGCGGAGAGGACGGGACCCAGAAATGGGCGGG | VHRRSHTGERPLQCEICGFTCRQRGNLLR |
| | | AAGCCGCGCAGAAAGAATCCCCAAGAGGGCCTGT | HIRLHTGERPFRCHLCNYACQRRDALSGS |
| | | ACAACGAGCTCCAAAAGGATAAGATGGCAGAAGC | GSGSGMSASSQSPNPNNPAEYCSTIPPLQQ |
| | | CTATAGCGAGATTGGTATGAAAGGGGAACGCAGA | AQASGALSSPPPTVMVPVGVLRHPGAEVA |
| | | AGAGGCAAAGGCCACGACGGACTGTACCAGGGAC | QPRSGGGGEGRGSLLTCGDVEENPGPRM |
| | | TCAGCACCGCCACCAAGGACACCTATGACGCTCTT | * (SEQ ID NO: 118) |
| | | CACATGCAGGCCCTGCCGCCTCGGGGTTCAGGTGC | |
| | | AACGAACTTCTCATTGTTGAAGCAAGCCGGTGATG | |
| | | TTGAGGAAAATCCGGGTCCTATGggaggccttGATTAC | |
| | | AAGGACGACGACGACAAGgctagcTTCAATGTCTTA | |
| | | ATGGTTCATcggCGAAGCCATACTGGTGAACGCCC | |
| | | ATTGCAGTGCGAAATATGCGGCTTTACCTGCCGCC | |
| | | AGcgcGGTAACCTCCTCCGCCACATTcgtCTGCACAC | |
| | | AGGGGAAcggCCTTTTcggTGTCACCTCTGCAACTAT | |
| | | GCATGCCAAAGAAGAGATGCGCTCAGCGGGTCAG | |
| | | GTAGTGGCtccggaATGAGCGCGTCATCCCAGAGCCC | |
| | | TAACCCAAATAATCCCGCAGAATACTGCAGCACA | |
| | | ATACCCCCTTTGCAGCAGGCACAAGCTAGTGGCGC | |
| | | TTTGAGCAGCCCACCGCCCACGGTCATGGTTCCTG | |
| | | TGGGAGTCCTTAgACATCCAGGGGCTGAAGTAGCA | |
| | | CAGCCCCGGTCCGGAGGCGGCGGAGAGGGCAGAG | |
| | | GAAGTCTTCTAACATGCGGTGACGTGGAGGAGAA | |
| | | TCCCGGCCCTAGGATG * (SEQ ID NO: 117) | |
| pJL_119 | c-mycNLS- | ATGcctgctgctaagagagtgaaactggatctcgagggaagcGAG | MPAAKRVKLDEGSENPLEMFETVPVWR |
| | DNMT3L- | AATCCCTTGGAAATGTTTGAAACCGTGCCTGTTTGGAGA | RQPVRVLSLFEDIKKELTSLGFLESGSDPG |
| | SOCS2- | AGACAACCGGTGCGCGTACTTTCATTGTTCGAAGA | QLKHVVDVTDTVRKDVEEWGPFDLVYGA |
| | P2A-CAR19 | CATTAAAAAGGAGCTCACCAGCCTTGGGTTCCTCG | TPPLGHTCDRPPSWYLFQFHRLLQYARPK |
| | | AGAGTGGAAGCGATCCTGGTCAGTTGAAACACGT | PGSPRPFFWMFVDNLVLNKEDLDVASRFL |
| | | GGTTGACGTCACAGACACGGTGAGGAAGGATGTT | EMEPVTIPDVHGGSLQNAVRVWSNIPAIRS |
| | | GAGGAGTGGGGTCCATTCGATTTGGTCTATGGCGC | RHWALVSEEELSLLAQNKQSSKLAAKWP |
| | | AACCCCTCCGCTTGGACATACGTGCGATAGACCTC | TKLVKNCFLPLREYFKYFSTELTSSLGSGS |
| | | CGAGCTGGTATCTTTTCCAGTTTCACAGGCTGCTT | GSGAPRNGTVHLYLTKPLYTSAPSLQHLC |
| | | CAATACGCAAGACCGAAGCCGGGTTCCCCTCGGC | RLTINKCTGAIWWGLPLPRLKDYLEEYKFQ |
| | | CCTTCTTCTGGATGTTTGTCGATAATCTCGTTTTGA | VGSGATNFSLLKQAGDVEENPGPMALPVT |
| | | ATAAAGAGGATTTGGATGTAGCATCCCGATTCCTT | ALLLPLALLLHAARPEQKLISEEDLDIQMT |
| | | GAAATGGAACCGGTAACGATCCCCGATGTTCATG | QTTSSLSASLGDRVTISCRASQDISKYLNW |
| | | GTGGGAGCTTGCAGAACGCTGTCCGGGTGTGGAG | YQQKPDGTVKLLIYHTSRLHSGVPSRFSGS |
| | | TAACATACCTGCCATCCGGTCAAGGCACTGGGCTC | GSGTDYSLTISNLEQEDIATYFCQQGNTLP |
| | | TCGTAAGTGAGGAAGAACTCTCACTGCTGGCGCA | YTFGGGTKLEIRGSTSGSGKPGSGEGSTKG |
| | | GAACAAGCAGTCTTCTAAATTGGCGGCAAAATGG | EVKLQESGPGLVAPSQSLSVTCTVSGVSLP |
| | | CCCACCAAGCTTGTAAAAATTGCTTTCTGCCCCT | DYGVSWIRQPPRKGLEWLGVIWGSETTYY |
| | | TCGAGAGTATTTTAAGTACTTTAGTACTGAACTCA | NSALKSRLTIIKDNSKSQVFLKMNSLQTDD |
| | | CATCATCACTGGGCAGTGGTAGTGGCtccggaGCTCC | TAIYYCAKHYYYGGSYAMDYWGQGTSV |
| | | ACGAAACGGCACGGTCCACCTTTACTTGACGAAA | TVSS * (SEQ ID NO: 120) |
| | | CCGCTTTATACCTCCGCACCGTCCCTCCAGCACCT | |
| | | GTGTCGGCTGACAATCAATAAGTGTACAGGTGCG | |
| | | ATATGGGGATTGCCGCTTCCGACCCGCCTTAAAGA | |
| | | TTACCTCGAGGAATACAAATTTCAAGTCGGTTCAG | |
| | | GTGCAACGAACTTCTCATTGTTGAAGCAAGCCGGT | |
| | | GATGTTGAGGAAAATCCGGGTCCTATGGCTTTGCC | |
| | | TGTTACTGCGCTTCTTTTGCCTTTGGCATTGTTGCT | |
| | | TCACGCCGCCAGGCCCGAGCAGAAGCTGATCAGC | |
| | | GAGGAGGACCTGGACATACAGATGACGCAAACAA | |
| | | CTTCCAGTCTTAGCGCTAGCCTGGGGGATCGAGTC | |
| | | ACCATATCTTGCAGGGCGTCcCAAGACATTAGCAA | |
| | | GTATCTCAATTGGTATCAACAGAAACCTGATGGAA | |
| | | CAGTTAAACTTCTGATTTACCACACGAGTCGCCTG | |
| | | CACTCCGGTGTGCCCTCCAGATTCTCCGGCTCAGG | |
| | | AAGTGGAACCGACTACTCTCTCACCATCTCCAACC | |
| | | TCGAACAAGAAGACATAGCTACATACTTTTGCCAA | |
| | | CAAGGTAATACCCTCCCCTATACCTTCGGTGGAGG | |
| | | CACTAAGCTGGAGATCACAGGGAGCACGTCCGGG | |
| | | TCTGGCAAACCGGGGAGTGGTGAGGGGTCTACGA | |
| | | AGGGAGAAGTCAAGCTTCAGGAGTCAGGACCCGG | |
| | | TCTTGTAGCTCCCAGCCAAAGCCTGTCAGTTACAT | |
| | | GCACGGTTTCCGGTGTGTCTTTGCCAGATTATGGC | |
| | | GTATCTTGGATTCGCCAACCGCCTAGAAAGGGACT | |
| | | TGAGTGGTTGGGTGTCATTTGGGGATCAGAAACA | |
| | | ACTTACTATAACAGTGCTCTTAAGTCCAGGTTGAC | |
| | | TATAATCAAGGACAATAGTAAGTCCCAAGTTTTTC | |
| | | TGAAAATGAATTCCCTGCAGACAGATGACACCGC | |
| | | TATCTACTACTGTGCCAAGCACTACTATTATGGGG | |
| | | GCTCTTATGCTATGGACTATTGGGGTCAGGGGACA | |
| | | TCAGTTACTGTTTCCAGC * (SEQ ID NO: 119) | |
| pJL_120 | CAR19- | TACTGTAAGCGCGGTCGGAAGAAGCTGCTGTACA | YCKRGRKKLLYIFKQPFMRPVQTTQEEDG |
| | P2A- | TCTTTAAGCAACCCTTCATGAGGCCTGTGCAGACT | CSCRFPEEEEGGCELRVKFSRSASAPAYQ |
| | cmycNLS- | ACTCAAGAGGAGGACGGCTGTTCATGCCGGTTCCC | QGQNQLYNELNLGRREEYDVLDKRRGRD |
| | DNMT3L- | AGAGGAGGAGGAAGGCGGCTGCGAACTGCGCGTG | PEMGGKPRRKNPQEGLYNELQKDKMAEA |
| | SOCS2-T2A | AAATTCAGCCGCAGCGCAGATGCTCCAGCCTACC | YSEIGMKGERRRGKGHDGLYQGLSTATK |
| | | AACAGGGGCAGAACCAGCTCTACAACGAACTCAA | DTYDALHMQALPPRGSGATNFSLLKQAG |
| | | TCTTGGTCGGAGAGAGGAGTACGACGTGCTGGAC | DVEENPGPMPAAKRVKLDLEGSENPLEMF |
| | | AAGCGGAGAGGACGGGACCCAGAAATGGGCGGG | ETVPVWRRQPVRVLSLFEDIKKELTSLGFL |
| | | AAGCCGCGCAGAAAGAATCCCCAAGAGGGCCTGT | ESGSDPGQLKHVVDVTDTVRKDVEEWGP |
| | | ACAACGAGCTCCAAAAGGATAAGATGGCAGAAGC | FDLVYGATPPLGHTCDRPPSWYLFQFHRL |
| | | CTATAGCGAGATTGGTATGAAAGGGGAACGCAGA | LQYARPKPGSPRPFFWMFVDNLVLNKEDL |
| | | AGAGGCAAAGGCCACGACGGACTGTACCAGGGAC | DVASRFLEMEPVTIPDVHGGSLQNAVRV |
| | | TCAGCACCGCCACCAAGGACACCTATGACGCTCTT | WSNIPAIRSRHWALVSEEELSLLQANKQSS |
| | | CACATGCAGGCCCTGCCGCCTCGGGGTTCAGGTGC | KLAAKWPTKLVKNCFLPLREYFKYFSTEL |
| | | AACGAACTTCTCATTGTTGAAGCAAGCCGGTGATG | TSSLGSGSGSGAPRNGTVHLYLTKPLYTSA |
| | | TTGAGGAAAATCCGGGTCCTATGcctgctgctaagagagtga | PSLQHLCRLTINKCTGAIWGLPLPTRLKDY |
| | | aactggatctcgagggaagcGAGAATCCCTTGGAAATGTTTG | LEEYKFQVSGGGGEGRGSLLTCGDVEENP |
| | | AAACCGTGCCTGTTTGGAGAAGACAACCGGTGCG | GPRM * (SEQ ID NO: 122) |
| | | CGTACTTTCATTGTTCGAAGACATTAAAAAGGAGC | |
| | | TCACCAGCCTTGGGTTCCTCGAGAGTGGAAGCGAT | |
| | | CCTGGTCAGTTGAAACACGTGGTTGACGTCACAGA | |
| | | CACGGTGAGGAAGGATGTTGAGGAGTGGGGTCCA | |
| | | TTCGATTTGGTCTATGGCGCAACCCCTCCGCTTGG | |
| | | ACATACGTGCGATAGACCTCCGAGCTGGTATCTTT | |
| | | TCCAGTTTCACAGGCTGCTTCAATACGCAAGACCG | |
| | | AAGCCGGGTTCCCCTCGGCCCTTCTTCTGGATGTT | |
| | | TGTCGATAATCTCGTTTTGAATAAAGAGGATTTGG | |
| | | ATGTAGCATCCCGATTCCTTGAAATGGAACCGGTA | |
| | | ACGATCCCCGATGTTCATGGTGGGAGCTTGCAGAA | |
| | | CGCTGTCCGGGTGTGGAGTAACATACCTGCCATCC | |
| | | GGTCAAGGCACTGGGCTCTCGTAAGTGAGGAAGA | |
| | | ACTCTCACTGCTGGCGCAGAACAAGCAGTCTTCTA | |
| | | AATTGGCGGCAAAATGGCCCACCAAGCTTGTGAA | |
| | | AAATTGCTTTCTGCCCCTTCGAGAGTATTTTAAGT | |
| | | ACTTTAGTACTGAACTCACATCATCACTGGGCAGT | |
| | | GGTAGTGGCtccggaGCTCCACGAAACGGCACGGTC | |
| | | CACCTTTACTTGACGAAACCGCTTTATACCTCCGC | |
| | | ACCGTCCCTCCAGCACCTGTGTCGGCTGACAATCA | |
| | | ATAAGTGTACAGGTGCGATATGGGGATTGCCGCTT | |
| | | CCGACCCGCCTTAAAGATTACCTCGAGGAATACA | |
| | | AATTTCAAGTCTCCGGAGGCGGCGGAGAGGGCAG | |
| | | AGGAAGTCTTCTAACATGCGGTGACGTGGAGGAG | |
| | | AATCCCGGCCCTAGGATG * (SEQ ID NO: 121) | |
| pJL_125 | DNMT1 | AAGGGCGGCGGCGGCTCCGGCGGCGGCGGCTCCA | KGGGGSGGGGSKPKSQACEPSEPEIEIKLP |
| | | AGCCCAAGTCCCAAGCCTGCGAACCATCCGAACC | KLRTLDVFSGCGGLSEGFHQAGISDTLWAI |
| | | TGAAATCGAGATCAAACTTCCGAAACTGAGGACG | EMWDPAAQAFRLNNPGSTVFTEDCNILLK |
| | | CTCGACGTCTTCTCTGGCTGCGGAGGACTGTCTGA | LVMAGETTNSRGQRLPQKGDVEMLCGGP |
| | | AGGCTTCCACCAAGCGGGTATAAGTGACACGCTCT | PCQGFSGMNRFNSRTYSKFKNSLVVSFLS |
| | | GGGCGATCGAAATGTGGGACCCTGCTGCCCAGGC | YCDYYRPRFFLLENVRNFVSFKRSMVLKL |
| | | GTTTAGGTTGAATAACCCTGGTTCTACAGTCTTTA | TLRCLVRMGYQCTFGVLQAGQYGVAQTR |
| | | CAGAAGATTGCAATATACTCCTCAAATTGGTGATG | RRAIILAAAPGEKLPLFPEPLHVFAPRACQ |
| | | GCAGGAGAAACTACTAACTCACGAGGGCAACGGC | LSVVVDDKKFVSNITRLSSGPFRTITVRDT |
| | | TCCCTCAGAAGGGAGATGTCGAGATGCTTTGCGG | MSDLPEVRNGASALEISYNGEPQSWFQRQ |
| | | GGGGCCGCCATGCCAAGGCTTTTCCGGAATGAAC | LRGAQYQPRILRDHICKDMSALVAARMRHI |
| | | CGATTCAATTCAAGGACATATTCTAAGTTTAAAAA | PLAPGSDWRDLPNIEVRLSDGTMARKLRY |
| | | CAGTTTGGTAGTAAGTTTTCTGAGCTACTGTGATT | THHDRKNGRSSSGALRGVCSCVEAGKAC |
| | | ACTATCGACCAAGATTTTTCCTCCTGGAGAACGTC | DPAARQFNTLIPWCLPHTGNRHNHWAGL |
| | | CGGAATTTTGTAAGTTTCAAGAGAAGCATGGTCTT | YGRLEWDGFFSTTVTVNPEPMGKQGRVLH |
| | | GAAATTGACCCTTCGCTGCTTGGTGAGGATGGGGT | PEQHRVVSVRECARSQGFPDTYRLFGNIL |
| | | ACCAGTGTACTTTTGGCGTGCTCCAAGCGGGACAG | DKHRQVGNAVPPPLAKAIGLEIKLCMLAK |
| | | TACGGTGTTGCCCAAACTCGGCGGCGAGCTATCAT | ARESASAKIKEEEAAKD* * (SEQ ID |
| | | CCTCGCAGCTGCACCCGGTGAAAAGTTGCCGCTTT | NO: 124) |
| | | TTCCTGAACCGCTGCACGTATTCGCGCCTAGGGCG | |
| | | TGTCAGCTTTCAGTCGTTGTCGATGATAAGAAATT | |
| | | CGTATCAAACATCACCCGACTCTCTTCCGGCCCGT | |
| | | TTCGGACGATTACAGTGCGCGACACGATGTCAGA | |
| | | CTTGCCCGAGGTTAGGAACGGCGCCTCAGCACTCG | |
| | | AAATCTCATACAACGGTGAACCACAATCATGGTTC | |
| | | CAGCGGCAGCTTCGCGGTGCACAATATCAGCCCAT | |
| | | ACTCCGAGACCACATCTGCAAGGACATGAGCGCC | |
| | | CTGGTAGCAGCTCGGATGCGGCATATACCTCTTGC | |
| | | GCCGGGCTCTGATTGGAGAGATCTCCCTAACATAG | |
| | | AGGTCCGGCTTTCTGATGGGACGATGGCCCGGAA | |
| | | ACTGCGATATACGCATCACGACCGGAAGAACGGT | |
| | | CGGAGTAGTTCTGGTGCCTTGCGCGGGGTGTGCTC | |
| | | TTGCGTGGAGGCCGGAAAGGCTTGCGATCCTGCC | |
| | | GCTCGGCAATTCAATACGCTGATTCCTTGGTGCCT | |
| | | TCCGCATACCGGGAATAGGCACAATCATTGGGCT | |
| | | GGACTTTATGGAAGACTCGAATGGGACGGTTTTTT | |
| | | TAGTACAACCGTTACTAACCCAGAACCAATGGGC | |
| | | AAGCAGGGCCGGGTTTTGCACCCAGAGCAGCACA | |
| | | GAGTCGTATCTGTGAGGGAGTGCGCTCGAAGCCA | |
| | | GGGCTTCCCAGATACTTACAGGCTCTTTGGTAATA | |
| | | TTCTGGATAAACACCGCCAAGTCGGAAACGCGGT | |
| | | CCCACCGCCTCTCGCCAAGGCCATTGGCCTCGAGA | |
| | | TCAAGCTGTGTATGCTCGCCAAGGCTAGGGAATCT | |
| | | GCTTCCGCAAAGATAAAGGAAGAAGAAGCTGCGA | |
| | | AAGATTAA * (SEQ ID NO: 123) | |
| pJL_126 | DNMT3B | AAGGGCGGCGGCGGCTCCGGCGGCGGCGGCTCCA | KGGGGSGGGGSTSDTGLEYEAPKLYPAIP |
| | | CTTCCGACACTGGACTTGAATACGAAGCCCCGAA | AARRRPIRVLSLFDGIATGYLVLKELGIKV |
| | | GCTCTACCCAGCTATCCCAGCCGCACGCCGGAGAC | GKYVASEVCEESIAVGTVKHEGNIKYVND |
| | | CGATTAGGGTCCTCTCTCTGTTTGATGGAATCGCA | VRNITKKNIEEWGPFDLVIGGSPCNDLSNV |
| | | ACAGGATACTTGGTACTGAAGGAACTCGGGATTA | NPARKGLYEGTGRLFFEFYHLLNYSRPKE |
| | | AAGTTGGGAAGTATGTAGCGAGTGAGGTGTGCGA | GDDRPFFWMFENVVAMKVGDKRDISRFL |
| | | GGAAAGCATTGCTGTTGGGACAGTAAAACACGAA | ECNPVMIDAIKVSAAHRARYFWGNLPGM |
| | | GGCAACATTAAGTACGTCAATGATGTAAGGAATA | NRPVIASKNDKLELQDCLEYNRIAKLKKV |
| | | TTACTAAAAAGAACATAGAAGAATGGGGGCCTTT | QTITTKSNSIKQGKNQLFPVVMNGKEDVL |
| | | CGACCTCGTGATAGGAGGCAGTCCATGCAATGAT | WCTELERIFGFPVHYTDVSNMGRGARQKL |
| | | CTCTCCAACGTTAATCCTGCAAGGAAGGGCCTGTA | LGRSWSVPVIRHLFAPLKDYFACE* * (SEQ |
| | | TGAAGGAACAGGCCGGCTGTTCTTCGAGTTTTACC | ID NO: 126) |
| | | ACTTGCTGAATTACTCTCGACCAAAAGAAGGTGAT | |
| | | GATAGACCCTTCTTCTGGATGTTTGAAAACGTTGT | |
| | | CGCTATGAAGGTCGGGGATAAGCGCGACATCAGT | |
| | | CGATTCTTGGAGTGTAACCCTGTGATGATAGATGC | |
| | | AATCAAGGTTAGTGCCGCGCACAGAGCGAGATAT | |
| | | TTCTGGGGAAATCTGCCGGGGATGAACCGCCCCGT | |
| | | GATAGCTAGTAAGAACGATAAGTTGGAGCTCCAA | |
| | | GATTGTCTTGAATATAATAGGATCGCCAAATTGAA | |
| | | GAAGGTCCAAACAATCACAACGAAAAGTAATTCC | |
| | | ATAAAACAGGGAAAAAATCAACTCTTCCCCGTCG | |
| | | TTATGAACGGCAAAGAGGATGTTCTTTGGTGCACG | |
| | | GAACTGGAAAGGATCTTCGGGTTTCCAGTGCATTA | |
| | | TACAGATGTTAGTAACATGGGAAGGGGAGCGCGC | |
| | | CAGAAGCTGTTGGGGAGATCCTGGAGTGTACCAG | |
| | | TTATTCGACACTTGTTCGCGCCTTTGAAAGATTAC | |
| | | TTTGCTTGTGAATAA * (SEQ ID NO: 125) | |
| pJL_127 | FMC63- | TACTGTAGGAGTAAGAGGAGCAGGCTCCTGCACA | YCRSKRSRLLHSDYMNMTPRRPGPTRKH |
| | CD28-CD3z | GTGACTACATGAACATGACTCCTAGAAGGCCTGG | YQPYAPPRDFAAYRSRVKFSRSADAPAYQ |
| | | ACCCACCCGCAAGCATTACCAGCCCTATGCCCCAC | QGQNQLYNELNLGRREEYDVLDKRRGRD |
| | | CACGCGACTTCGCAGCCTATCGCTCCCGCGTGAAA | PEMGGKPRRKNPQEGLYNELQKDKMAEA |
| | | TTCAGCCGCAGCGCAGATGCTCCAGCCTACCAACA | YSEIGMKGERRRGKGHDGLYQGLSTATK |
| | | GGGGCAGAACCAGCTCTACAACGAACTCAATCTT | DTYDALHMQALPPRSGGGGEGRGSLLTC |
| | | GGTCGGAGAGAGGAGTACGACGTGCTGGACAAGC | GDVEENPGPRM * (SEQ ID NO: 128) |
| | | GGAGAGGACGGGACCCAGAAATGGGGGGAAGC | |
| | | CGCGCAGAAAGAATCCCCAAGAGGGCCTGTACAA | |
| | | CGAGCTCCAAAAGGATAAGATGGCAGAAGCCTAT | |
| | | AGCGAGATTGGTATGAAAGGGGAACGCAGAAGAG | |
| | | GCAAAGGCCACGACGGACTGTACCAGGGACTCAG | |
| | | CACCGCCACCAAGGACACCTATGACGCTCTTCACA | |
| | | TGCAGGCCCTGCCGCCTCGGTCCGGAGGCGGCGG | |
| | | AGAGGGCAGAGGAAGTCTTCTAACATGCGGTGAC | |
| | | GTGGAGGAGAATCCCGGCCCTAGGATG * (SEQ ID | |
| | | NO: 127) | |
| pJL_128 | FMC63- | TACTGTAGGAGTAAGAGGAGCAGGCTCCTGCACA | YCRSKRSRLLHSDYMNMTPRRGPTRKH |
| | CD28-CD3z- | GTGACTACATGAACATGACTCCTAGAAGGCCTGG | YQPYAPPRDFAAYRSRVKFSRSADAPAYQ |
| | c-mycNLS- | ACCCACCCGCAAGCATTACCAGCCCTATGCCCCAC | QGQNQLYNELNLGRREEYDVLDKRRGRD |
| | DNMT3L- | CACGCGACTTCGCAGCCTATCGCTCCCGCGTGAAA | PEMGGKPRRKNPQEGLYNELQKDKMAEA |
| | SOC82 | TTCAGCCGCAGCGCAGATGCTCCAGCCTACCAACA | YSEIGMKGERRRGKGHDGLYQGLSTATK |
| | | GGGGCAGAACCAGCTCTACAACGAACTCAATCTT | DTYDALHMQALPPRGSGATNFSLLKQAG |
| | | GGTCGGAGAGAGGAGTACGACGTGCTGGACAAGC | DVEENPGPMPAAKRVKLDLEGSENPLEMF |
| | | GGAGAGGACGGGACCCAGAAATGGGCGGGAAGC | ETVPVWRRQPVRVLSLFEDIKKELTSLGFL |
| | | CGCGCAGAAAGAATCCCCAAGAGGGCCTGTACAA | ESGSDPGQLKHVVDVTDTVRKDVEEWP |
| | | CGAGCTCCAAAAGGATAAGATGGCAGAAGCCTAT | FDLVYGATPPLGHTCDRPPSWYLFQFHRL |
| | | AGCGAGATTGGTATGAAAGGGGAACGCAGAAGAG | LQYARPKPGSPRPFFWMFVDNLVLNKEDL |
| | | GCAAAGGCCACGACGGACTGTACCAGGGACTCAG | DVASRFLEMEPVTIPDVHGGSLQNAVRV |
| | | CACCGCCACCAAGGACACCTATGACGCTCTTCACA | WSNIPAIRSRHWALVSEEELSLLAQNKQSS |
| | | TGCAGGCCCTGCCGCCTCGGGGTTCAGGTGCAACG | KLAAKWPTKLVKNCFLPLREYFKYFSTEL |
| | | AACTTCTCATTGTTGAAGCAAGCCGGTGATGTTGA | TSSLGSGSGSGAPRNGTVHLYLTKPLYTSA |
| | | GGAAAATCCGGGTCCTATGcctgctgctaagagagtgaaactgg | PSLQHLCRLTINKCTGAIWGLPLPTRLKDY |
| | | atctcgagggaagcGAGAATCCCTTGGAAATGTTTGAAAC | LEEYKFQVSGGGGEGRGSLLTCGDVEENP |
| | | CGTGCCTGTTTGGAGAAGACAACCGGTGCGCGTA | GPRM * (SEQ ID NO: 130) |
| | | CTTTCATTGTTCGAAGACATTAAAAAGGAGCTCAC | |
| | | CAGCCTTGGGTTCCTCGAGAGTGGAAGCGATCCTG | |
| | | GTCAGTTGAAACACGTGGTTGACGTCACAGACAC | |
| | | GGTGAGGAAGGATGTTGAGGAGTGGGGTCCATTC | |
| | | GATTTGGTCTATGGCGCAACCCCTCCGCTTGGACA | |
| | | TACGTGCGATAGACCTCCGAGCTGGTATCTTTTCC | |
| | | AGTTTCACAGGCTGCTTCAATACGCAAGACCGAA | |
| | | GCCGGGTTCCCCTCGGCCCTTCTTCTGGATGTTTGT | |
| | | CGATAATCTCGTTTTGAATAAAGAGGATTTGGATG | |
| | | TAGCATCCCGATTCCTTGAAATGGAACCGGTAACG | |
| | | ATCCCCGATGTTCATGGTGGGAGCTTGCAGAACGC | |
| | | TGTCCGGGTGTGGAGTAACATACCTGCCATCCGGT | |
| | | CAAGGCACTGGGCTCTCGTAAGTGAGGAAGAACT | |
| | | CTCACTGCTGGCGCAGAACAAGCAGTCTTCTAAAT | |
| | | TGGCGGCAAAATGGCCCACCAAGCTTGTGAAAAA | |
| | | TTGCTTTCTGCCCCTTCGAGAGTATTTTAAGTACTT | |
| | | TAGTACTGAACTCACATCATCACTGGGCAGTGGTA | |
| | | GTGGCtccggaGCTCCACGAAACGGCACGGTCCACCT | |
| | | TTACTTGACGAAACCGCTTTATACCTCCGCACCGT | |
| | | CCCTCCAGCACCTGTGTCGGCTGACAATCAATAAG | |
| | | TGTACAGGTGCGATATGGGGATTGCCGCTTCCGAC | |
| | | CCGCCTTAAAGATTACCTCGAGGAATACAAATTTC | |
| | | AAGTCTCCGGAGGCGGCGGAGAGGGCAGAGGAA | |
| | | GTCTTCTAACATGCGGTGACGTGGAGGAGAATCCC | |
| | | GGCCCTAGGATG * (SEQ ID NO: 129) | |
| pJL_142 | aEGFR | ATGGCTTTGCCTGTTACTGCGCTTCTTTTGCCTTTG | MALPVTALLLPLALLLHAARPEQKLISEED |
| | | GCATTGTTGCTTCACGCCGCCAGGCCCGAGCAGAA | LQVQLKQSGPGLVQPSQSLSITCTVSGFSL |
| | | GCTGATCAGCGAGGAGGACCTGCAGGTACAGTTG | TNYGVHWVRQSPGKGLEWLGVIWSGGNT |
| | | AAACAAAGTGGTCCAGGGCTTGTTCAGCCTTCCCA | DYNTPFTSRLSINKDNSKSQVFFKMNSLQS |
| | | GAGTCTTAGCATCACATGCACGGTATCAGGGTTTT | NDTAIYYCARALTYYDYEFAYWGQGTLV |
| | | CCCTTACAAACTACGGAGTCCACTGGGTGCGGCA | TVSAGGGGSGGGGSGGGGSGGGGSDILLT |
| | | GTCTCCAGGTAAGGGTTTGGAATGGTTGGGGGTA | QSPVILSVSPGERVSFSCRASQSIGTNIHWY |
| | | ATCTGGTCTGGCGGAAATACGGACTATAACACCCC | QQRTNGSPRLLIKYASESISGIPSRFSGSGS |
| | | ATTTACCTCTCGCCTGTCAATCAATAAAGACAATT | GTDFTLSINSVESEDIADYYCQQNNNWPT |
| | | CAAAATCTCAGGTGTTCTTCAAGATGAATAGCCTC | TFGAGTKLELK (SEQ ID NO: 132) |
| | | CAGTCAAACGACACAGCAATATATTACTGCGCCA | |
| | | GAGCCCTCACGTATTATGATTATGAATTTGCTTAT | |
| | | TGGGGTCAAGGTACTCTTGTAACAGTAAGTGCAG | |
| | | GGGGTGGGGGTTCAGGAGGTGGTGGCTCCGGAGG | |
| | | AGGCGGGTCTGGAGGCGGAGGTAGCGATATTCTG | |
| | | CTTACCCAATCACCCGTTATTCTTAGCGTGTCCCC | |
| | | GGGTGAAAGAGTATCATTTAGTTGCAGGGCCTCTC | |
| | | AATCCATCGGAACGAACATCCATTGGTATCAACA | |
| | | ACGCACCAATGGGAGCCCGCGGCTCCTCATAAAA | |
| | | TATGCTAGCGAGTCCATCAGTGGCATACCGTCAAG | |
| | | ATTCAGCGGATCAGGTAGCGGAACCGATTTTACAC | |
| | | TCTCAATAAACTCAGTTGAAAGTGAGGATATTGCT | |
| | | GATTATTATTGTCAACAGAATAACAATTGGCCGAC | |
| | | AACTTTTGGCGCTGGCACCAAACTTGAACTGAAG | |
| | | (SEQ ID NO: 131) | |
| pJL_143 | SARA- | ATGAGCGCGTCATCCCAGAGCCCTAACCCAAATA | MSASSQSPNPNNPAEYCSTIPPLQQAQASG |
| | SOCS2 P2A | ATCCCGCAGAATACTGCAGCACAATACCCCCTTTG | ALSSPPPTVMVPVGVLKHPGAEVAQPRGS |
| | aEGFR | CAGCAGGCACAAGCTAGTGGCGCTTTGAGCAGCC | GSGSGAPRNGTVHLYLTKPLYTSAPSLQH |
| | | CACCGCCCACGGTCATGGTTCCTGTGGGAGTCCTT | LCRLTINKCTGAIWGLPLPTRLKDYLEEYK |
| | | AAACATCCAGGGGCTGAAGTAGCACAGCCCCGGG | FQVGSGATNFSLLKQAGDVEENPGPMALP |
| | | GCAGTGGTAGTGGCtccggaGCTCCACGAAACGGCA | VTALLLPLALLLHAARPEQKLISEEDLQVQ |
| | | CGGTCCACCTTTACTTGACGAAACCGCTTTATACC | LKQSGPGLVQPSQSLSITCTVSGFSLTNYG |
| | | TCCGCACCGTCCCTCCAGCACCTGTGTCGGCTGAC | VHWVRQSPGKGLEWLGVIWSGGNTDYNT |
| | | AATCAATAAGTGTACAGGTGCGATATGGGGATTG | PFTSRLSINKDNSKSQVFFKMNSLQSNDTA |
| | | CCGCTTCCGACCCGCCTTAAAGATTACCTCGAGGA | IYYCARALTYYDYEFAYWGQGTLVTVSA |
| | | ATACAAATTTCAAGTCGGTTCAGGTGCAACGAACT | GGGGSGGGGSGGGGSGGGGSDILLTQSPV |
| | | TCTCATTGTTGAAGCAAGCCGGTGATGTTGAGGAA | ILSVSPGERVSFSCRASQSIGTNIHWYQQR |
| | | AATCCGGGTCCTATGGCTTTGCCTGTTACTGCGCT | TNGSPRLLIKYASESISGIPSRFSGSGSGTDF |
| | | TCTTTTGCCTTTGGCATTGTTGCTTCACGCCGCCAG | TLSINSVESEDIADYYCQQNNNWPTTFGA |
| | | GCCCGAGCAGAAGCTGATCAGCGAGGAGGACCTG | GTKLELK (SEQ ID NO: 134) |
| | | CAGGTACAGTTGAAACAAAGTGGTCCAGGGCTTG | |
| | | TTCAGCCTTCCCAGAGTCTTAGCATCACATGCACG | |
| | | GTATCAGGGTTTTCCCTTACAAACTACGGAGTCCA | |
| | | CTGGGTGCGGCAGTCTCCAGGTAAGGGTTTGGAAT | |
| | | GGTTGGGGGTAATCTGGTCTGGCGGAAATACGGA | |
| | | CTATAACACCCCATTTACCTCTCGCCTGTCAATCA | |
| | | ATAAAGACAATTCAAAATCTCAGGTGTTCTTCAAG | |
| | | ATGAATAGCCTCCAGTCAAACGACACAGCAATAT | |
| | | ATTACTGCGCCAGAGCCCTCACGTATTATGATTAT | |
| | | GAATTTGCTTATTGGGGTCAAGGTACTCTTGTAAC | |
| | | AGTAAGTGCAGGGGGTGGGGGTTCAGGAGGTGGT | |
| | | GGCTCCGGAGGAGGCGGGTCTGGAGGCGGAGGTA | |
| | | GCGATATTCTGCTTACCCAATCACCCGTTATTCTTA | |
| | | GCGTGTCCCCGGGTGAAAGAGTATCATTTAGTTGC | |
| | | AGGGCCTCTCAATCCATCGGAACGAACATCCATTG | |
| | | GTATCAACAACGCACCAATGGGAGCCCGCGGCTC | |
| | | CTCATAAAATATGCTAGCGAGTCCATCAGTGGCAT | |
| | | ACCGTCAAGATTCAGCGGATCAGGTAGCGGAACC | |
| | | GATTTTACACTCTCAATAAACTCAGTTGAAAGTGA | |
| | | GGATATTGCTGATTATTATTGTCAACAGAATAACA | |
| | | ATTGGCCGACAACTTTTGGCGCTGGCACCAAACTT | |
| | | GAACTGAAG (SEQ ID NO: 133) | |
| pJL_144 | PD-1 stop | ATGCAAATCCCACAGGCTCCATGGCCAGTAGTCTG | MQIPQAPWPVVWAVLQLGWRPGWFLDSP |
| | | GGCGGTCTTGCAGCTTGGGTGGCGACCTGGTTGGT | DRPWNPPTFSPALLVVTEGDNATFTCSFSN |
| | | TTCTTGACTCACCTGACCGCCCCTGGAATCCACCT | TSESFVLNWYRMSPSNQTDKLAAFPEDRS |
| | | ACTTTTTCACCCGCGCTGCTGGTGGTGACCGAGGG | QPGQDCRFRVTQLPNGRDFHMSVVRARR |
| | | CGATAATGCTACTTTCACGTGTAGTTTTTCAAATA | NDSGTYLCGAISLAPKAQIKESLRAELRVT |
| | | CGTCCGAATCTTTCGTATTGAACTGGTACCGAATG | ERRAEVPTAHPSPSPRPAGQFQTLVVGVV |
| | | TCTCCTTCTAATCAAACCGATAAACTCGCAGCCTT | GGLLGSLVLLVWVLAVICSRAARGTIGAR |
| | | TCCAGAAGACAGATCCCAACCAGGACAAGACTGT | RTGQPLKEDPSAVPVFSVDYGELDFQWRE |
| | | CGCTTCCGCGTGACCCAACTTCCCAATGGTCGCGA | KTPEPPVPCVPEQTEYATIVFPSGMGTSSP |
| | | TTTCCATATGAGCGTAGTCAGAGCCAGAAGGAAT | ARRGSADGPRSAQPLRPEDGHCSWPL* |
| | | GATTCCGGGACTTATTTGTGTGGGGCCATCTCTCT | (SEQ ID NO: 136) |
| | | TGCCCCCAAGGCTCAGATTAAAGAGAGTCTGAGA | |
| | | GCGGAGCTGCGGGTAACGGAAAGACGCGCTGAAG | |
| | | TACCGACAGCGCATCCTAGCCCTTCTCCACGACCG | |
| | | GCCGGCCAATTTCAGACTTTGGTGGTAGGTGTGGT | |
| | | AGGCGGTCTGTTGGGGAGCCTGGTTCTCCTCGTGT | |
| | | GGGTACTGGCAGTGATCTGTTCTCGCGCCGCACGA | |
| | | GGAACCATCGGTGCAAGAAGGACTGGCCAACCAC | |
| | | TTAAGGAGGACCCATCTGCTGTGCCTGTTTTTTCT | |
| | | GTAGATTATGGAGAACTCGACTTCCAATGGAGAG | |
| | | AAAAAACTCCCGAACCACCGGTACCCTGTGTCCCC | |
| | | GAACAAACGGAATATGCTACGATCGTGTTTCCAA | |
| | | GCGGTATGGGGACTTCCAGTCCCGCAAGGCGAGG | |
| | | GTCAGCCGACGGTCCACGGAGTGCACAACCATTG | |
| | | AGGCCAGAAGATGGACACTGTTCCTGGCCGCTTtaa | |
| | | (SEQ ID NO: 135) | |
| pJL_145 | BTLA stop | ATGAAGACTTTGCCGGCGATGTTGGGAACTGGTA | MKTLPAMLGTGKLFWVFFLIPYLDIWNIH |
| | | AGTTGTTTTGGGTGTTCTTTCTTATTCCTTATCTTG | GKESCDVQLYIKRQSEHSILAGDPFELECP |
| | | ACATTTGGAATATCCACGGGAAAGAGTCATGTGA | VKYCANRPHVTWCKLNGTTCVKLEDRQT |
| | | TGTCCAACTTTATATCAAGCGACAAAGCGAACATT | SWKEEKNISFFILHFEPVLPNDNGSYRCSA |
| | | CCATCCTTGCCGGCGATCCATTCGAGCTGGAATGC | NFQSNLIESHSTTLYVTDVKSASERPSKDE |
| | | CCCGTAAAGTATTGCGCGAATCGCCCTCACGTTAC | MASRPWLLYRLLPLGGLPLLITTCFCLFCC |
| | | CTGGTGCAAATTGAATGGAACGACGTGTGTTAAAT | LRRHQGKQNELSDTAGREINLVDAHLKSE |
| | | TGGAGGACCGGCAGACTTCTTGGAAAGAAGAGAA | QTEASTRQNSQVLLSETGIYDNDPDLCFR |
| | | AAATATCTCTTTTTTCATTCTGCATTTCGAGCCAGT | MQEGSEVYSNPCLEENKPGIVYASLNHSVI |
| | | TCTTCCGAATGATAATGGCTCATATCGGTGCTCCG | GPNSRLARNVKEAPTEYASICVRS* (SEQ |
| | | CCAACTTCCAGAGCAATCTCATAGAGAGCCACTCC | ID NO: 138) |
| | | ACTACGCTTTATGTCACTGACGTTAAGTCAGCTTC | |
| | | AGAGCGCCCGAGTAAAGACGAAATGGCATCACGG | |
| | | CCCTGGCTTCTGTATCGCTTGCTCCCACTCGGGGG | |
| | | ATTGCCGCTCCTCATCACAACTTGTTTCTGCTTGTT | |
| | | CTGCTGTTTGCGAAGGCACCAGGGGAAACAAAAT | |
| | | GAATTGTCTGACACGGCGGGACGCGAGATAAACC | |
| | | TGGTGGATGCGCATCTGAAGTCAGAACAGACAGA | |
| | | AGCAAGTACCCGCCAGAACTCCCAGGTCCTGCTGT | |
| | | CTGAAACTGGTATATACGACAACGACCCCGACTTG | |
| | | TGTTTCCGGATGCAAGAGGGATCTGAAGTCTATAG | |
| | | TAACCCGTGCCTCGAAGAGAATAAACCGGGCATC | |
| | | GTGTATGCCTCTCTCAATCATTCCGTTATTGGACC | |
| | | AAACTCTAGGCTCGCAAGAAATGTTAAAGAAGCA | |
| | | CCTACGGAGTATGCAAGCATTTGCGTTCGGTCCtaa | |
| | | (SEQ ID NO: 137) | |
| pJL_146 | 2B4 stop | ATGCTCGGCCAAGTTGTAACTCTCATACTCCTTCTT | MLGQVVTLILLLLLKVYQGKGCQGSADH |
| | SHP1-SOCS | CTGCTGAAAGTTTACCAAGGGAAGGGGTGCCAAG | VVSISGVPLQLQPNSIQTKVDSIAWKKLLP |
| | | GTTCTGCGGACCATGTAGTGAGCATCTCAGGAGTC | SQNGFHHILKWENGSLPSNTSNDRFSFIVK |
| | | CCGCTTCAGCTCCAGCCGAATAGTATCCAAACAAA | NLSLLIKAAQQQDSGLYCLEVTSISGKVQT |
| | | GGTGGACAGCATAGCTTGGAAGAAGTTGTTGCCCT | ATFQVFVFESLLPDKVEKPRLQGQGKILDR |
| | | CTCAAAATGGCTTTCATCACATACTGAAATGGGAA | GRCQVALSCLVSRDGNVSYAWYRGSKLI |
| | | AATGGCAGCTTGCCTTCAAACACATCCAATGACAG | QTAGNLTYLDEEVDINGTHTYTCNVSNPV |
| | | ATTTTCATTCATAGTTAAAAACCTTTCCCTTCTTAT | SWESHTLNLTQDCQNAHQEFRFWPFLVII |
| | | TAAGGCGGCTCAGCAGCAAGACTCAGGACTCTAC | VILSALFLGTLACFCVWRRKRKEKQSETSP |
| | | TGTCTCGAAGTAACATCCATCAGTGGAAAGGTCCA | KEFLTIYEDVKDLKTRRNHEQEQTFPGGG |
| | | GACGGCTACATTTCAGGTCTTTGTTTTTGAGTCTCT | STIYSMIQSQSSAPTSQEPAYTLYSLIQPSR |
| | | CTTGCCTGACAAGGTTGAAAAACCGAGACTCCAA | KSGSRKRNHSPSFNSTIYEVIGKSQPKAQN |
| | | GGACAGGGTAAAATTCTGGATCGGGGACGATGCC | PARLSRKELENFDVYS (SEQ ID NO: 140) |
| | | AGGTAGCACTTAGTTGCCTCGTATCTCGGGATGGA | |
| | | AACGTATCCTATGCTTGGTACCGCGGATCAAAACT | |
| | | GATACAAACTGCTGGAAATTTGACCTACCTGGACG | |
| | | AGGAAGTTGATATAAATGGAACTCACACTTATACT | |
| | | TGTAACGTTTCAAATCCGGTCAGCTGGGAATCTCA | |
| | | TACACTCAATCTCACTCAAGATTGCCAAAATGCGC | |
| | | ATCAAGAGTTTAGATTTTGGCCGTTTCTCGTAATA | |
| | | ATCGTGATCCTGTCTGCCCTGTTCCTCGGAACGCT | |
| | | GGCTTGTTTTTGCGTGTGGCGGCGCAAAAGGAAG | |
| | | GAAAAGCAGAGCGAAACGAGCCCGAAGGAGTTCC | |
| | | TTACAATCTATGAGGACGTAAAAGATTTGAAAAC | |
| | | CAGGCGGAACCATGAACAAGAACAGACCTTTCCG | |
| | | GGAGGTGGAAGTACCATTTACTCCATGATACAATC | |
| | | ACAATCATCCGCGCCTACGTCCCAAGAACCCGCCT | |
| | | ATACTCTGTATTCTCTCATTCAGCCTAGCCGGAAA | |
| | | AGTGGTAGCCGAAAGCGGAACCACTCCCCTTCATT | |
| | | CAACTCCACTATTTACGAAGTAATCGGCAAATCTC | |
| | | AGCCCAAAGCACAAAACCCAGCACGCCTGTCAAG | |
| | | GAAGGAGCTGGAGAACTTCGATGTTTACAGTtaa | |
| | | (SEQ ID NO: 139) | |
| pJL_147 | SHP1-SOCS | ATGGTCCGCTGGTTTCATAGAGACCTGTCCGGGCT | MVRWFHRDLSGLDAETLLKGRGVHGSFL |
| | degrader | TGATGCGGAGACCTTGCTGAAGGGTCGAGGAGTT | ARPSRKNQGDFSLSVRVGDQVTHIRIQNS |
| | | CATGGCAGCTTCCTCGCTAGACCTTCACGGAAAAA | GDFYDLYGGEKFATLTELVEYYTQQQGV |
| | | TCAGGGGGACTTTAGTCTTTCCGTGAGAGTGGGTG | LQDRDGTIIHLKYPLNCSDPTSERWYHGH |
| | | ACCAGGTTACGCACATTAGGATCCAAAATAGTGG | MSGGQAETLLQAKGEPWTFLVRESLSQPG |
| | | TGACTTCTATGATCTTTATGGTGGTGAGAAATTCG | DFVLSVLSDQPKAGPGSPLRVTHIKVMCE |
| | | CCACACTCACAGAGCTTGTTGAGTACTATACCCAA | GGRYTVGGLETFDSLTDLVEHFKKTGIEE |
| | | CAACAAGGGGTGTTGCAGGACCGAGATGGAACGA | ASGAFVYLRQPYYATRVNGSGSGSGAPRN |
| | | TCATTCACCTGAAGTACCCCCTGAATTGCTCCGAT | GTVHLYLTKPLYTSAPSLQHLCRLTINKCT |
| | | CCTACGTCCGAAAGGTGGTATCATGGTCATATGTC | GAIWGLPLPTRLKDYLEEYKFQV (SEQ ID |
| | | CGGCGGACAAGCCGAGACACTTCTCCAAGCTAAG | NO: 142) |
| | | GGTGAGCCGTGGACCTTCTTGGTTAGAGAGAGCCT | |
| | | GTCCCAGCCAGGTGATTTCGTACTGTCCGTTTTGA | |
| | | GTGACCAGCCCAAAGCTGGACCGGGTAGCCCATT | |
| | | GAGGGTCACCCATATTAAGGTCATGTGTGAGGGC | |
| | | GGGCGATATACAGTGGGCGGTCTCGAAACCTTTG | |
| | | ACAGCTTGACCGATCTCGTTGAGCACTTCAAAAAA | |
| | | ACAGGAATAGAAGAAGCGAGTGGCGCGTTTGTAT | |
| | | ATCTGCGACAACCCTATTACGCAACCAGGGTCAAT | |
| | | GGCAGTGGTAGTGGCtccggaGCTCCACGAAACGGC | |
| | | ACGGTCCACCTTTACTTGACGAAACCGCTTTATAC | |
| | | CTCCGCACCGTCCCTCCAGCACCTGTGTCGGCTGA | |
| | | CAATCAATAAGTGTACAGGTGCGATATGGGGATT | |
| | | GCCGCTTCCGACCCGCCTTAAAGATTACCTCGAGG | |
| | | AATACAAATTTCAAGTC (SEQ ID NO: 141) | |
| pJL_148 | SHP2-SOCS | ATGACGTCAAGGCGCTGGTTCCACCCGAACATCAC | MTSRRWFHPNITGVEAENLLLTRGVDGSF |
| | degrader | GGGAGTCGAGGCGGAGAACTTGCTTCTCACTAGA | LARPSKSNPGDFTLSVRRNGAVTHIKIQNT |
| | | GGTGTAGACGGGTCCTTTCTCGCCCGACCTTCCAA | GDYYDLYGGEKFATLAELVQYYMEHHG |
| | | AAGTAATCCAGGGGACTTCACACTGTCCGTCCGGC | QLKEKNGDVIELKYPLNCADPTSERWFHG |
| | | GCAATGGCGCCGTTACCCACATCAAAATTCAAAAT | HLSGKEAEKLLTEKGKHGSFLVRESQSHP |
| | | ACAGGAGATTACTATGATCTCTACGGAGGCGAAA | GDFVLSVRTGDDKGESNDGKSKVTHVMI |
| | | AGTTCGCGACGCTCGCAGAACTCGTACAGTACTAT | RCQELKYDVGGGERFDSLTDLVEHYKKN |
| | | ATGGAGCATCATGGTCAACTGAAAGAAAAGAATG | PMVETLGTVLQLKQPLNTTRINAAEGSGS |
| | | GCGACGTTATCGAATTGAAATACCCCTTGAACTGT | GSGAPRNGTVHLYLTKPLYTSAPSLQHLC |
| | | GCGGATCCTACGTCTGAACGATGGTTCCATGGTCA | RLTINKCTGAIWGLPLPTRLKDYLEEYKFQ |
| | | TCTTAGTGGCAAGGAAGCTGAGAAGCTTCTCACG | V (SEQ ID NO: 144) |
| | | GAAAAAGGGAAGCATGGTTCCTTCCTTGTTAGGG | |
| | | AAAGTCAATCTCATCCCGGGGACTTCGTCCTGTCA | |
| | | GTGAGAACTGGAGATGATAAGGGCGAGTCTAATG | |
| | | ATGGTAAATCTAAGGTGACCCATGTGATGATTAGA | |
| | | TGCCAAGAGTTGAAGTACGATGTGGGAGGAGGCG | |
| | | AGAGATTCGACTCATTGACTGATCTGGTCGAACAT | |
| | | TATAAAAAGAACCCCATGGTAGAAACGTTGGGTA | |
| | | CAGTTCTCCAGTTGAAACAGCCTTTGAACACGACT | |
| | | CGGATAAATGCTGCGGAGGGCAGTGGTAGTGGCtc | |
| | | cggaGCTCCACGAAACGGCACGGTCCACCTTTACTT | |
| | | GACGAAACCGCTTTATACCTCCGCACCGTCCCTCC | |
| | | AGCACCTGTGTCGGCTGACAATCAATAAGTGTACA | |
| | | GGTGCGATATGGGGATTGCCGCTTCCGACCCGCCT | |
| | | TAAAGATTACCTCGAGGAATACAAATTTCAAGTC | |
| | | (SEQ ID NO: 143) | |
| pJL_151 | FMC63- | TACTGTAGGAGTAAGAGGAGCAGGCTCCTGCACA | YCRSKRSRLLHSDYMNMTPRRPGPTRKH |
| | CD28-CD3z- | GTGACTACATGAACATGACTCCTAGAAGGCCTGG | YQPYAPPRDFAAYRSRVKFSRSADAPAYQ |
| | SV40NLS- | ACCCACCCGCAAGCATTACCAGCCCTATGCCCCAC | QGQNQLYNELNLGRREEYDVLDKRRGRD |
| | DNMT3L- | CACGCGACTTCGCAGCCTATCGCTCCCGCGTGAAA | PEMGGKPRRKNPQEGLYNELQKDKMAEA |
| | SOCS2 | TTCAGCCGCAGCGCAGATGCTCCAGCCTACCAACA | YSEIGMKGERRRGKGHDGLYQGLSTATK |
| | | GGGGCAGAACCAGCTCTACAACGAACTCAATCTT | DTYDALHMQALPPRGSGATNFSLLKQAG |
| | | GGTCGGAGAGAGGAGTACGACGTGCTGGACAAGC | DVEENPGPMAPKKKRKVGIHGVPAAGSE |
| | | GGAGAGGACGGGACCCAGAAATGGGCGGGAAGC | NPLEMFETVPVWRRQPVRVLSLFEDIKKE |
| | | CGCGCAGAAAGAATCCCCAAGAGGGCCTGTACAA | LTSLGFLESGSDPGQLKHVVDVTDTVRKD |
| | | CGAGCTCCAAAAGGATAAGATGGCAGAAGCCTAT | VEEWGPFDLVYGATPPLGHTCDRPPSWYL |
| | | AGCGAGATTGGTATGAAAGGGGAACGCAGAAGAG | FQFHRLLQYARPKPGSPRPFFWMFVDNLV |
| | | GCAAAGGCCACGACGGACTGTACCAGGGACTCAG | LNKEDLDVASRFLEMEPVTIPDVHGGSLQ |
| | | CACCGCCACCAAGGACACCTATGACGCTCTTCACA | NAVRVWSNIPAIRSRHWALVSEEELSLLA |
| | | TGCAGGCCCTGCCGCCTCGGGGTTCAGGTGCAACG | QNKQSSKLAAKWPTKLVKNCFLPLREYFK |
| | | AACTTCTCATTGTTGAAGCAAGCCGGTGATGTTGA | YFSTELTSSLGSGSGSGAPRNGTVHLYLTK |
| | | GGAAAATCCGGGTCCTATGgccccaaagaagaagcggaaggt | PLYTSAPSLQHLCRLTINKCTGAIWGLPLP |
| | | cggtatccacggagtcccagcagccggaagcGAGAATCCCTTGGA | TRLKDYLEEYKFQVSGGGGEGRGSLLTCG |
| | | AATGTTTGAAACCGTGCCTGTTTGGAGAAGACAAC | DVEENPGPRM (SEQ ID NO: 298) |
| | | CGGTGCGCGTACTTTCATTGTTCGAAGACATTAAA | |
| | | AAGGAGCTCACCAGCCTTGGGTTCCTCGAGAGTG | |
| | | GAAGCGATCCTGGTCAGTTGAAACACGTGGTTGA | |
| | | CGTCACAGACACGGTGAGGAAGGATGTTGAGGAG | |
| | | TGGGGTCCATTCGATTTGGTCTATGGCGCAACCCC | |
| | | TCCGCTTGGACATACGTGCGATAGACCTCCGAGCT | |
| | | GGTATCTTTTCCAGTTTCACAGGCTGCTTCAATAC | |
| | | GCAAGACCGAAGCCGGGTTCCCCTCGGCCCTTCTT | |
| | | CTGGATGTTTGTCGATAATCTCGTTTTGAATAAAG | |
| | | AGGATTTGGATGTAGCATCCCGATTCCTTGAAATG | |
| | | GAACCGGTAACGATCCCCGATGTTCATGGTGGGA | |
| | | GCTTGCAGAACGCTGTCCGGGTGTGGAGTAACAT | |
| | | ACCTGCCATCCGGTCAAGGCACTGGGCTCTCGTAA | |
| | | GTGAGGAAGAACTCTCACTGCTGGCGCAGAACAA | |
| | | GCAGTCTTCTAAATTGGCGGCAAAATGGCCCACCA | |
| | | AGCTTGTGAAAAATTGCTTTCTGCCCCTTCGAGAG | |
| | | TATTTTAAGTACTTTAGTACTGAACTCACATCATC | |
| | | ACTGGGCAGTGGTAGTGGCtccggaGCTCCACGAAA | |
| | | CGGCACGGTCCACCTTTACTTGACGAAACCGCTTT | |
| | | ATACCTCCGCACCGTCCCTCCAGCACCTGTGTCGG | |
| | | CTGACAATCAATAAGTGTACAGGTGCGATATGGG | |
| | | GATTGCCGCTTCCGACCCGCCTTAAAGATTACCTC | |
| | | GAGGAATACAAATTTCAAGTCTCCGGAGGCGGCG | |
| | | GAGAGGGCAGAGGAAGTCTTCTAACATGCGGTGA | |
| | | CGTGGAGGAGAATCCCGGCCCTAGGATG (SEQ ID | |
| | | NO: 297) | |
| pJL_152 | FMC63- | TACTGTAGGAGTAAGAGGAGCAGGCTCCTGCACA | YCRSKRSRLLHSDYMNMTPRRPGPTRKH |
| | CD28-CD3z- | GTGACTACATGAACATGACTCCTAGAAGGCCTGG | YQPYAPPRDFAAYRSRVKFSRSADAPAYQ |
| | DNMT3L- | ACCCACCCGCAAGCATTACCAGCCCTATGCCCCAC | QGQNQLYNELNLGRREEYDVLDKRRGRD |
| | SOCS2- | CACGCGACTTCGCAGCCTATCGCTCCCGCGTGAAA | PEMGGKPRRKNPQEGLYNELQKDKMAEA |
| | NPLNLS | TTCAGCCGCAGCGCAGATGCTCCAGCCTACCAACA | YSEIGMKGERRRGKGHDGLYQGLSTATK |
| | | GGGGCAGAACCAGCTCTACAACGAACTCAATCTT | DTYDALHMQALPPRGSGATNFSLLKQAG |
| | | GGTCGGAGAGAGGAGTACGACGTGCTGGACAAGC | DVEENPGPMGSENPLEMFETVPVWRRQP |
| | | GGAGAGGACGGGACCCAGAAATGGGCGGGAAGC | VRVLSLFEDIKKELTSLGFLESGSDPGQLK |
| | | CGCGCAGAAAGAATCCCCAAGAGGGCCTGTACAA | HVVDVTDTVRKDVEEWGPFDLVYGATPP |
| | | CGAGCTCCAAAAGGATAAGATGGCAGAAGCCTAT | LGHTCDRPPSWYLFQFHRLLQYARPKPGS |
| | | AGCGAGATTGGTATGAAAGGGGAACGCAGAAGAG | PRPFFWMFVDNLVLNKEDLDVASRFLEM |
| | | GCAAAGGCCACGACGGACTGTACCAGGGACTCAG | EPVTIPDVHGGSLQNAVRVWSNIPAIRSRH |
| | | CACCGCCACCAAGGACACCTATGACGCTCTTCACA | WALVSEEELSLLAQNKQSSKLAAKWPTK |
| | | TGCAGGCCCTGCCGCCTCGGGGTTCAGGTGCAACG | LVKNCFLPLREYFKYFSTELTSSLGSGSGS |
| | | AACTTCTCATTGTTGAAGCAAGCCGGTGATGTTGA | GAPRNGTVHLYLTKPLYTSAPSLQHLCRL |
| | | GGAAAATCCGGGTCCTATGggaagcGAGAATCCCTT | TINKCTGAIWGLPLPTRLKDYLEEYKFQV |
| | | GGAAATGTTTGAAACCGTGCCTGTTTGGAGAAGA | GGGGSKRPAATKKAGQAKKKKSGGGGE |
| | | CAACCGGTGCGCGTACTTTCATTGTTCGAAGACAT | GRGSLLTCGDVEENPGPRM (SEQ ID NO: |
| | | TAAAAAGGAGCTCACCAGCCTTGGGTTCCTCGAG | 300) |
| | | AGTGGAAGCGATCCTGGTCAGTTGAAACACGTGG | |
| | | TTGACGTCACAGACACGGTGAGGAAGGATGTTGA | |
| | | GGAGTGGGGTCCATTCGATTTGGTCTATGGCGCAA | |
| | | CCCCTCCGCTTGGACATACGTGCGATAGACCTCCG | |
| | | AGCTGGTATCTTTTCCAGTTTCACAGGCTGCTTCA | |
| | | ATACGCAAGACCGAAGCCGGGTTCCCCTCGGCCCT | |
| | | TCTTCTGGATGTTTGTCGATAATCTCGTTTTGAATA | |
| | | AAGAGGATTTGGATGTAGCATCCCGATTCCTTGAA | |
| | | ATGGAACCGGTAACGATCCCCGATGTTCATGGTGG | |
| | | GAGCTTGCAGAACGCTGTCCGGGTGTGGAGTAAC | |
| | | ATACCTGCCATCCGGTCAAGGCACTGGGCTCTCGT | |
| | | AAGTGAGGAAGAACTCTCACTGCTGGCGCAGAAC | |
| | | AAGCAGTCTTCTAAATTGGCGGCAAAATGGCCCA | |
| | | CCAAGCTTGTGAAAAATTGCTTTCTGCCCCTTCGA | |
| | | GAGTATTTTAAGTACTTTAGTACTGAACTCACATC | |
| | | ATCACTGGGCAGTGGTAGTGGCtccggaGCTCCACGA | |
| | | AACGGCACGGTCCACCTTTACTTGACGAAACCGCT | |
| | | TTATACCTCCGCACCGTCCCTCCAGCACCTGTGTC | |
| | | GGCTGACAATCAATAAGTGTACAGGTGCGATATG | |
| | | GGGATTGCCGCTTCCGACCCGCCTTAAAGATTACC | |
| | | TCGAGGAATACAAATTTCAAGTCGGCGGCGGCGG | |
| | | CTCCaagcgtcctgctgctactaagaaagctggtcaagctaagaaa | |
| | | aagaaaTCCGGAGGCGGCGGAGAGGGCAGAGGAAGTCTTCT | |
| | | AACATGCGGTGACGTGGAGGAGAATCCCGGCCCT | |
| | | AGGATG (SEQ ID NO: 299) | |
| pJL-209 | SARA- | AAACTGGGGCACAAGCTTAATGGTTCA | KLGHKLNGSGATNFSLLKQAGD |
| | SOCS2 | GGTGCAACGAACTTCTCATTGTTGAAGC | VEENPGPMSASSQSPNPNNPAEY |
| | | AAGCCGGTGATGTTGAGGAAAATCCGG | CSTIPPLQQAQASGALSSPPPTVM |
| | | GTCCTATGAGCGCGTCATCCCAGAGCCC | VPVGVLKHPGAEVAQPRGSGSGS |
| | | TAACCCAAATAATCCCGCAGAATACTGC | GAPRNGTVHLYLTKPLYTSAPSL |
| | | AGCACAATACCCCCTTTGCAGCAGGCAC | QHLCRLTINKCTGAIWGLPLPTRL |
| | | AAGCTAGTGGCGCTTTGAGCAGCCCACC | KDYLEEYKFQV* (SEQ ID NO: 302) |
| | | GCCCACGGTCATGGTTCCTGTGGGAGTC | |
| | | CTTAAACATCCAGGGGCTGAAGTAGCA | |
| | | CAGCCCCGGGGCAGTGGTAGTGGCtccgg | |
| | | aGCTCCACGAAACGGCACGGTCCACCTT | |
| | | TACTTGACGAAACCGCTTTATACCTCCG | |
| | | CACCGTCCCTCCAGCACCTGTGTCGGCT | |
| | | GACAATCAATAAGTGTACAGGTGCGAT | |
| | | ATGGGGATTGCCGCTTCCGACCCGCCTT | |
| | | AAAGATTACCTCGAGGAATACAAATTTC | |
| | | AAGTCTAA (SEQ ID NO: 301) | |

TABLE 9
Sequence Engineered SSR Sequences

| Element (binding | | |
|---|---|---|
| partner where relevant) | Sequence (M) = start Met) | Domain/function |
| vhhGFP4 (GFP) | (M)GGLDYKDDDDKASDQVQLVESGGALVQ | Target binding domain |
| | PGGSLRLSCAASGFPVNRYSMRWYRQAPGK | or target polypeptide |
| | EREWVAGMSSAGDRSSYEDSVKGRFTISRDD | |
| | ARNTVYLQMNSLKPEDTAVYYCNVNVGFEY | |
| | WGQGTQVTVSS (SEQ ID NO: 145) | |
| DNMT3A (DNMT3L) | SEQ ID NO: 50 | Target binding domain |
| | | or target polypeptide |
| SMAD2 (SARA) | SEQ ID NO: 108 | Target polypeptide or |
| | | target polypeptide |
| PD-1 (SHP1/2) | SEQ ID NO: 38 | Target binding domain |
| | | or target polypeptide |
| BTLA (SHP1/2) | SEQ ID NO: 138 | Target binding domain |
| | | or target polypeptide |
| 2B4 (SHP1/2) | SEQ ID NO: 140 | Target binding domain |
| | | or target polypeptide |
| TET2 (IDAX) | SEQ ID NO: 46 | Target binding domain |
| | | or target polypeptide |
| TET2 (IDAX) | SEQ ID NO: 48 | Target binding domain |
| | | or target polypeptide |
| DNMT3L (DNMT3A) | (M)GSENPLEMFETVPVWRRQPVRVLSLFEDI | Target binding domain |
| | KKELTSLGFLESGSDPGQLKHVVDVTDTVRK | or target polypeptide |
| | DVEEWGPFDLVYGATPPLGHTCDRPPSWYL | |
| | FQFHRLLQYARPKPGSPRPFFWMFVDNLVLN | |
| | KEDLDVASRFLEMEPVTIPDVHGGSLQNAVR | |
| | VWSNIPAIRSRHWALVSEEELSLLAQNKQSS | |
| | KLAAKWPTKLVKNCFLPLREYFKYFSTELTS | |
| | SL (SEQ ID NO: 146) | |
| IDAX (TET2) | (M)HHRNDSQRLGKAGCPPEPSLQMANTNFL | Target binding domain |
| | STLSPEHCRPLAGECMNKLKCGAAEAEIMNL | or target polypeptide |
| | PERVGTFSAIPALGGISLPPGVIVMTALHSPA | |
| | AASAAVTDSAFQIANLADCPQNHSSSSSSSSG | |
| | GAGGANPAKKKRKRCGVCVPCKRLINCGVC | |
| | SSCRNRKAGAAICKFRKCEELKKKPGTSLER | |
| | TPVPSAEAFRWFF | |
| | (SEQ ID NO: 147) | |
| IDAX (TET2) | SEQ ID NO: 76 | Target binding domain |
| | | or target polypeptide |
| SARA (SMAD2/3) | (M)SASSQSPNPNNPAEYCSTIPPLQQAQASG | Target binding domain |
| | ALSSPPPTVMVPVGVLKHPGAEVAQPR (SEQ | or target polypeptide |
| | ID NO: 148) | |
| SMAD3 (SARA) | SEQ ID NO: 110 | Target binding domain |
| | | or target polypeptide |
| DNMT1 | SEQ ID NO: 124 | Target binding domain |
| | | or target polypeptide |
| DNMT3B | SEQ ID NO: 126 | Target binding domain |
| | | or target polypeptide |
| aEFGR | SEQ ID NO: 132 | Target binding domain |
| | | or target polypeptide |
| PD-1 (SHP1/2) | SEQ ID NO: 136 | Target binding domain |
| | | or target polypeptide |
| SHP1 (PD-1) | VRWFHRDLSGLDAETLLKGRGVHGSFLARP | Target binding domain |
| | SRKNQGDFSLSVRVGDQVTHIRIQNSGDFYD | or target polypeptide |
| | LYGGEKFATLTELVEYYTQQQGVLQDRDGTI | |
| | IHLKYPLNCSDPTSERWYHGHMSGGQAETLL | |
| | QAKGEPWTFLVRESLSQPGDFVLSVLSDQPK | |
| | AGPGSPLRVTHIK VMCEGGRYTVGGLETFDS | |
| | LTDLVEHFKKTGIEEASGAFVYLRQPYYATR | |
| | VN (SEQ ID NO: 149) | |
| SHP2 (PD-1) | TSRRWFHPNITGVEAENLLLTRGVDGSFLAR | Target binding domain |
| | PSKSNPGDFTLSVRRNGAVTHIKIQNTGDYY | or target polypeptide |
| | DLYGGEKFATLAELVQYYMEHHGQLKEKN | |
| | GDVIELKYPLNCADPTSERWFHGHLSGKEAE | |
| | KLLTEKGKHGSFLVRESQSHPGDFVLSVRTG | |
| | DDKGESNDGKSKVTHVMIRCQELKYDVGGG | |
| | ERFDSLTDLVEHYKKNPMVETLGTVLQLKQ | |
| | PLNTTRINAAE (SEQ ID NO: 150) | |
| bTrCP2 | DPAEAVLQEKALKFMNSSEREDCNNGEPPR | E3 ligase binding domain |
| | KIIPEKNSLRQTYNSCARLCLNQETVCLASTA | |
| | MKTENCVAKTKLANGTSSMIVPKQRKLSAS | |
| | CHYQHGHINSYLKPMLQRDFITALPARGLDH | |
| | YEKEKELCVKYFEQWSESDQVEFVEHLISQM | |
| | CHYQHGHINSYLKPMLQRDFITALPARGLDH | |
| | IAENILSYLDAKSLCAAELVCKEWYRVTSDG | |
| | MLWKKLIERMVRTDSLWRGLAERRGWGQY | |
| | LFKNKPPDGNAPPNSFYRALYPKIIQDIETIES | |
| | NWRCGRHSLQRIHCR (SEQ ID NO: 151) | |
| CHIP | RLNFGDDIPSALRIAKKKRWNSIEERRIHQES | E3 ligase binding domain |
| | ELHSYLSRLIAAERERELEECQRNHEGDEDD | |
| | SHVRAQQACIEAKHDKYMADMDELFSQVDE | |
| | KRKKRDIPDYLCGKISFELMREPCITPSGITYD | |
| | RKDIEEHLQRVGHFDPVTRSPLTQEQLIPNLA | |
| | MKEVIDAFISENGWVEDY (SEQ ID NO: 152) | |
| FBW7 | CVPRSGLILSCICLYCGVLLPVLLPNLPFLTCL | E3 ligase binding domain |
| | SMSTLESVTYLPEKGLYCQRLPSSRTHGGTES | |
| | LKGKNTENMGFYGTLKMIFYKMKRKLDHGS | |
| | EVRSFSLGKKPCKVSEYTSTTGLVPCSATPTT | |
| | FGDLRAANGQGQQRRRITSVQPPTGLQEWL | |
| | KMFQSWSGPEKLLALDELIDSCEPTQVKHM | |
| | MQVIEPQFQRDFISLLPKELALYVLSFLEPKD | |
| | LLQAAQTCRYWRILAEDNLLWREKCKEEGI | |
| | DEPLHIKRRK VIKPGFIHSPWKSAYIRQHRIDT | |
| | NWRRGELKSP (SEQ ID NO: 153) | |
| SOCS2 | APRNGTVHLYLTKPLYTSAPSLQHLCRLTIN | E3 ligase binding domain |
| | KCTGAIWGLPLPTRLKDYLEEYKFQV | |
| | (SEQ ID NO: 154) | |
| SPOP | SVNISGQNTMNMVKVPECRLADELGGLWEN | E3 ligase binding domain |
| | SRFTDCCLCVAGQEFQAHKAILAARSPVFSA | |
| | MFEHEMEESKKNRVEINDVEPEVFKEMMCFI | |
| | YTGKAPNLDKMADDLLAAADKYALERLKV | |
| | MCEDALCSNLSVENAAEILILADLHSADQLK | |
| | TQAVDFINYHASDVLETSGWKSMVVSHPHL | |
| | VAEAYRSLASAQCPFLGPPRKR (SEQ ID NO: | |
| | 155) | |
| VHL | GTLPVYTLKERCLQVVRSLVKPENYRRLDIV | E3 ligase binding domain |
| | RSLYEDLEDHPNVQKDLERLTQERIAHQRM | |
| | GD (SEQ ID NO: 156) | |
| VPR | EQAPEDQGPQREPHNEWTLELLEELKNEAVR | E3 ligase binding domain |
| | HFPRIWLHGLGQHIYETYGDTWAGVEAIIRIL | |
| | QQLLFIHFRIGCRHSRIGVTQQRRARNGASRS | |
| | (SEQ ID NO: 157) | |
| VPX | ADPRERVPPGNSGEETIGEAFEWLDRTIEALN | E3 ligase binding domain |
| | REAVNHLPRELIFQVWQRSWAYWHDEQGM | |
| | STSYTKYRYLCIMQKAVYIH (SEQ ID NO: | |
| | 158) | |
| WHVX | GPCCLDVTCAELRTMDSTVNFVSWHANRQL | E3 ligase binding domain |
| | G (SEQ ID NO: 159) | |
| ZnF913K0 | FNVLMVHRRSHTGERPLQCEICGFTCRQRGN | E3 ligase binding domain |
| | LLRHIRLHTGERPFRCHLCNYACQRRDAL | |
| | (SEQ ID NO: 160) | |
| Vpr | SEQ ID NO: 78 | E3 ligase binding domain |
| SKP2 | MHRKHLQEIPDLSSNVATSFTWGWDSSKTSE | E3 ligase binding domain |
| | LLSGMGVSALEKEEPDSENIPQELLSNLGHPE | |
| | SPPRKRLKSKGSDKDFVIVRRPKLNRENFPG | |
| | VSWDSLPDELLLGIFSCLCLPELLKVSGVCKR | |
| | WYRLASDESLWQTLDLTGKNLGSAGSAAGS | |
| | GEF (SEQ ID NO: 161) | |

### Base Expression Vectors

```
Cilantro2
(SEQ ID NO: 303)
AGGTGCCTCTGCGGCCAAAAGCCACGTGTATAAGATACACCTGCAAAGGCGGCACAACCCCAGTGCCACGTTGTGAGTTGGATAG
TTGTGGAAAGAGTCAAATGGCTCTCCTCAAGCGTATTCAACAAGGGGCTGAAGGATGCCCAGAAGGTACCCCATTGTATGGGATC
TGATCTGGGGCCTCGGTACACATGCTTTACATGTGTTTAGTCGAGGTTAAAAAAACGTCTAGGCCCCCCGAACCACGGGGACGTG
GTTTTCCTTTGAAAAACACGATGATAATATGGCCACAACCCTGGAATTCGCCACCATGGTGAGCAAGGGCGAGGAGGATAACATG
GCCATCATCAAGGAGTTCATGCGCTTCAAGGTGCACATGGAGGGCTCCGTGAACGGCCACGAGTTCGAGATCGAGGGCGAGGGC
GAGGGCCGCCCCTACGAGGGCACCCAGACCGCCAAGCTGAAGGTGACCAAGGGTGGCCCCCTGCCCTTCGCCTGGGACATCCTGT
CCCCTCAGTTCATGTACGGCTCCAAGGCCTACGTGAAGCACCCCGCCGACATCCCCGACTACTTGAAGCTGTCCTTCCCCGAGGGC
TTCAAGTGGGAGCGCGTGATGAACTTCGAGGACGGCGGCGTGGTGACCGTGACCCAGGACTCCTCCCTGCAGGACGGCGAGTTCA
TCTACAAGGTGAAGCTGCGCGGCACCAACTTCCCCTCCGACGGCCCCGTAATGCAGAAGAAGACCATGGGCTGGGAGGCCTCCTC
CGAGCGGATGTACCCCGAGGACGGCGCCCTGAAGGGCGAGATCAAGCAGAGGCTGAAGCTGAAGGACGGCGGCCACTACGACG
CTGAGGTCAAGACCACCTACAAGGCCAAGAAGCCCGTGCAGCTGCCCGGCGCCTACAACGTCAACATCAAGTTGGACATCACCTC
CCACAACGAGGACTACACCATCGTGGAACAGTACGAACGCGCCGAGGGCCGCCACTCCACCGGCGGCATGGACGAGCTGTACAA
GTAAACTAGTAAGCTTGGCGTAACTAGATCTTGAGACAAATGGCAGTATTCATCCACAATTTTAAAAGAAAAGGGGGGATTGGGG
GGTACAGTGCAGGGGAAAGAATAGTAGACATAATAGCAACAGACATACAAACTAAAGAATTACAAAAACAAATTACAAAAATTC
AAAATTTTCGGGTTTATTACAGGGACAGCAGAGATCCACTTTGGGCTCGAGGGGGCCCGGGTGCAAAGATGGATAAAGTTTTAAA
CAGAGAGGAATCTTTGCAGCTAATGGACCTTCTAGGTCTTGAAAGGAGTGGGAATTGGCTCCGGTGCCCGTCAGTGGGCAGAGCG
CACATCGCCCACAGTCCCCGAGAAGTTGGGGGGAGGGGTCGGCAATTGATCCGGTGCCTAGAGAAGGTGGCGCGGGGTAAACTG
GGAAAGTGATGTCGTGTACTGGCTCCGCCTTTTTCCCGAGGGTGGGGGAGAACCGTATATAAGTGCAGTAGTCGCCGTGAACGTT
CTTTTTCGCAACGGGTTTGCCGCCAGAACACAGGTAAGTGCCGTGTGTGGTTCCCGCGGGCCTGGCCTCTTTACGGGTTATGGCCC
TTGCGTGCCTTGAATTACTTCCACCTGGCTGCAGTACGTGATTCTTGATCCCGAGCTTCGGGTTGGAAGTGGGTGGGAGAGTTCGA
GGCCTTGCGCTTAAGGAGCCCCTTCGCCTCGTGCTTGAGTTGAGGCCTGGCCTGGGCGCTGGGGCCGCCGCGTGCGAATCTGGTG
GCACCTTCGCGCCTGTCTCGCTGCTTTCGATAAGTCTCTAGCCATTTAAAATTTTTGATGACCTGCTGCGACGCTTTTTTTCTGGCA
AGATAGTCTTGTAAATGCGGGCCAAGATCTGCACACTGGTATTTCGGTTTTTGGGGCCGCGGGCGGCGACGGGGCCCGTGCGTCC
CAGCGCACATGTTCGGCGAGGCGGGGCCTGCGAGCGCGGCCACCGAGAATCGGACGGGGGTAGTCTCAAGCTGGCCGGCCTGCT
CTGGTGCCTGGCCTCGCGCCGCCGTGTATCGCCCCGCCCTGGGCGGCAAGGCTGGCCCGGTCGGCACCAGTTGCGTGAGCGGAAA
GATGGCCGCTTCCCGGCCCTGCTGCAGGGAGCTCAAAATGGAGGACGCGGCGCTCGGGAGAGCGGGCGGGTGAGTCACCCACAC
AAAGGAAAAGGGCCTTTCCGTCCTCAGCCGTCGCTTCATGTGACTCCACGGAGTACCGGGCGCCGTCCAGGCACCTCGATTAGTT
CTCGAGCTTTTGGAGTACGTCGTCTTTAGGTTGGGGGGAGGGGTTTTATGCGATGGAGTTTCCCCACACTGAGTGGGTGGAGACTG
AAGTTAGGCCAGCTTGGCACTTGATGTAATTCTCCTTGGAATTTGCCCTTTTTGAGTTTGGATCTTGGTTCATTCTCAAGCCTCAGA
CAGTGGTTCAAAGTTTTTTTCTTCCATTTCAGGTGTCGTGACGTACGGCCACCATGACCGAGTACAAGCCCACGGTGCGCCTCGCC
ACCCGCGACGACGTCCCCAGGGCCGTACGCACCCTCGCCGCCGCGTTCGCCGACTACCCCGCCACGCGCCACACCGTCGATCCGG
ACCGCCACATCGAGCGGGTCACCGAGCTGCAAGAACTCTTCCTCACGCGCGTCGGGCTCGACATCGGCAAGGTGTGGGTCGCGGA
CGACGGCGCCGCCGTGGCGGTCTGGACCACGCCGGAGAGCGTCGAAGCGGGGGCGGTGTTCGCCGAGATCGGCCCGCGCATGGC
CGAGTTGAGCGGTTCCCGGCTGGCCGCGCAGCAACAGATGGAAGGCCTCCTGGCGCCGCACCGGCCCAAGGAGCCCGCGTGGTT
CCTGGCCACCGTCGGAGTCTCGCCCGACCACCAGGGCAAGGGTCTGGGCAGCGCCGTCGTGCTCCCCGGAGTGGAGGCGGCCGA
GCGCGCCGGGGTGCCCGCCTTCCTGGAGACCTCCGCGCCCCGCAACCTCCCCTTCTACGAGCGGCTCGGCTTCACCGTCACCGCCG
ACGTCGAGGTGCCCGAAGGACCGCGCACCTGGTGCATGACCCGCAAGCCCGGTGCCTGAACGCGTTAAGTCGACAATCAACCTCT
GGATTACAAAATTTGTGAAAGATTGACTGGTATTCTTAACTATGTTGCTCCTTTTACGCTATGTGGATACGCTGCTTTAATGCCTTT
GTATCATGCTATTGCTTCCCGTATGGCTTTCATTTTCTCCTCCTTGTATAAATCCTGGTTGCTGTCTCTTTATGAGGAGTTGTGGCCC
GTTGTCAGGCAACGTGGCGTGGTGTGCACTGTGTTTGCTGACGCAACCCCCACTGGTTGGGGCATTGCCACCACCTGTCAGCTCCT
TTCCGGGACTTTCGCTTTCCCCCTCCCTATTGCCACGGCGGAACTCATCGCCGCCTGCCTTGCCCGCTGCTGGACAGGGGCTCGGC
TGTTGGGCACTGACAATTCCGTGGTGTTGTCGGGGAAATCATCGTCCTTTCCTTGGCTGCTCGCCTGTGTTGCCACCTGGATTCTGC
GCGGGACGTCCTTCTGCTACGTCCCTTCGGCCCTCAATCCAGCGGACCTTCCTTCCCGCGGCCTGCTGCCGGCTCTGCGGCCTCTTC
CGCGTCTTCGCCTTCGCCCTCAGACGAGTCGGATCTCCCTTTGGGCCGCCTCCCCGCGTCGACTTTAAGACCAATGACTTACAAGG
CAGCTGTAGATCTTAGCCACTTTTTAAAAGAAAAGGGGGGACTGGAAGGGCTAATTCACTCCCAACGAAGACAAGATCTGCTTTT
TGCTTGTACTGGGTCTCTCTGGTTAGACCAGATCTGAGCCTGGGAGCTCTCTGGCTAACTAGGGAACCCACTGCTTAAGCCTCAAT
AAAGCTTGCCTTGAGTGCTTCAAGTAGTGTGTGCCCGTCTGTTGTGTGACTCTGGTAACTAGAGATCCCTCAGACCCTTTTAGTCA
GTGTGGAAAATCTCTAGCAGTACGTATAGTAGTTCATGTCATCTTATTATTCAGTATTTATAACTTGCAAAGAAATGAATATCAGA
GAGTGAGAGGAACTTGTTTATTGCAGCTTATAATGGTTACAAATAAAGCAATAGCATCACAAATTTCACAAATAAAGCATTTTTTT
CACTGCATTCTAGTTGTGGTTTGTCCAAACTCATCAATGTATCTTATCATGTCTGGCTCTAGCTATCCCGCCCCTAACTCCGCCCAT
CCCGCCCCTAACTCCGCCCAGTTCCGCCCATTCTCCGCCCCATGGCTGACTAATTTTTTTTATTTATGCAGAGGCCGAGGCCGCCTC
GGCCTCTGAGCTATTCCAGAAGTAGTGAGGAGGCTTTTTTGGAGGCCTAGGGACGTACCCAATTCGCCCTATAGTGAGTCGTATTA
CGCGCGCTCACTGGCCGTCGTTTTACAACGTCGTGACTGGGAAAACCCTGGCGTTACCCAACTTAATCGCCTTGCAGCACATCCCC
CTTTCGCCAGCTGGCGTAATAGCGAAGAGGCCCGCACCGATCGCCCTTCCCAACAGTTGCGCAGCCTGAATGGCGAATGGGACGC
GCCCTGTAGCGGCGCATTAAGCGCGGCGGGTGTGGTGGTTACGCGCAGCGTGACCGCTACACTTGCCAGCGCCCTAGCGCCCGCT
CCTTTCGCTTTCTTCCCTTCCTTTCTCGCCACGTTCGCCGGCTTTCCCCGTCAAGCTCTAAATCGGGGGCTCCCTTTAGGGTTCCGAT
TTAGTGCTTTACGGCACCTCGACCCCAAAAAACTTGATTAGGGTGATGGTTCACGTAGTGGGCCATCGCCCTGATAGACGGTTTTT
CGCCCTTTGACGTTGGAGTCCACGTTCTTTAATAGTGGACTCTTGTTCCAAACTGGAACAACACTCAACCCTATCTCGGTCTATTCT
TTTGATTTATAAGGGATTTTGCCGATTTCGGCCTATTGGTTAAAAAATGAGCTGATTTAACAAAAATTTAACGCGAATTTTAACAA
AATATTAACGCTTACAATTTAGGTGGCACTTTTCGGGGAAATGTGCGCGGAACCCCTATTTGTTTATTTTTCTAAATACATTCAAAT
ATGTATCCGCTCATGAGACAATAACCCTGATAAATGCTTCAATAATATTGAAAAAGGAAGAGTATGAGTATTCAACATTTCCGTG
TCGCCCTTATTCCCTTTTTTGCGGCATTTTGCCTTCCTGTTTTTGCTCACCCAGAAACGCTGGTGAAAGTAAAAGATGCTGAAGATC
AGTTGGGTGCACGAGTGGGTTACATCGAACTGGATCTCAACAGCGGTAAGATCCTTGAGAGTTTTCGCCCCGAAGAACGTTTTCC
AATGATGAGCACTTTTAAAGTTCTGCTATGTGGCGCGGTATTATCCCGTATTGACGCCGGGCAAGAGCAACTCGGTCGCCGCATAC
ACTATTCTCAGAATGACTTGGTTGAGTACTCACCAGTCACAGAAAAGCATCTTACGGATGGCATGACAGTAAGAGAATTATGCAG
TGCTGCCATAACCATGAGTGATAACACTGCGGCCAACTTACTTCTGACAACGATCGGAGGACCGAAGGAGCTAACCGCTTTTTTG
CACAACATGGGGGATCATGTAACTCGCCTTGATCGTTGGGAACCGGAGCTGAATGAAGCCATACCAAACGACGAGCGTGACACC
ACGATGCCTGTAGCAATGGCAACAACGTTGCGCAAACTATTAACTGGCGAACTACTTACTCTAGCTTCCCGGCAACAATTAATAG
ACTGGATGGAGGCGGATAAAGTTGCAGGACCACTTCTGCGCTCGGCCCTTCCGGCTGGCTGGTTTATTGCTGATAAATCTGGAGCC
GGTGAGCGTGGGTCTCGCGGTATCATTGCAGCACTGGGGCCAGATGGTAAGCCCTCCCGTATCGTAGTTATCTACACGACGGGGA
GTCAGGCAACTATGGATGAACGAAATAGACAGATCGCTGAGATAGGTGCCTCACTGATTAAGCATTGGTAACTGTCAGACCAAGT
TTACTCATATATACTTTAGATTGATTTAAAACTTCATTTTTAATTTAAAAGGATCTAGGTGAAGATCCTTTTTGATAATCTCATGAC
CAAAATCCCTTAACGTGAGTTTTCGTTCCACTGAGCGTCAGACCCCGTAGAAAAGATCAAAGGATCTTCTTGAGATCCTTTTTTTC
TGCGCGTAATCTGCTGCTTGCAAACAAAAAAACCACCGCTACCAGCGGTGGTTTGTTTGCCGGATCAAGAGCTACCAACTCTTTTT
CCGAAGGTAACTGGCTTCAGCAGAGCGCAGATACCAAATACTGTTCTTCTAGTGTAGCCGTAGTTAGGCCACCACTTCAAGAACT
CTGTAGCACCGCCTACATACCTCGCTCTGCTAATCCTGTTACCAGTGGCTGCTGCCAGTGGCGATAAGTCGTGTCTTACCGGGTTG
GACTCAAGACGATAGTTACCGGATAAGGCGCAGCGGTCGGGCTGAACGGGGGGTTCGTGCACACAGCCCAGCTTGGAGCGAACG
ACCTACACCGAACTGAGATACCTACAGCGTGAGCTATGAGAAAGCGCCACGCTTCCCGAAGGGAGAAAGGCGGACAGGTATCCG
GTAAGCGGCAGGGTCGGAACAGGAGAGCGCACGAGGGAGCTTCCAGGGGGAAACGCCTGGTATCTTTATAGTCCTGTCGGGTTTC
GCCACCTCTGACTTGAGCGTCGATTTTTGTGATGCTCGTCAGGGGGGCGGAGCCTATGGAAAAACGCCAGCAACGCGGCCTTTTT
ACGGTTCCTGGCCTTTTGCTGGCCTTTTGCTCACATGTTCTTTCCTGCGTTATCCCCTGATTCTGTGGATAACCGTATTACCGCCTTT
GAGTGAGCTGATACCGCTCGCCGCAGC
Chive
  (SEQ ID NO: 304)
CGAACGACCGAGCGCAGCGAGTCAGTGAGCGAGGAAGCGGAAGAGCGCCCAATACGCAAACCGCCTCTCCCCGCGCGTTGGCCG
ATTCATTAATGCAGCTGGCACGACAGGTTTCCCGACTGGAAAGCGGGCAGTGAGCGCAACGCAATTAATGTGAGTTAGCTCACTC
ATTAGGCACCCCAGGCTTTACACTTTATGCTTCCGGCTCGTATGTTGTGTGGAATTGTGAGCGGATAACAATTTCACACAGGAAAC
AGCTATGACCATGATTACGCCAAGCGCGCAATTAACCCTCACTAAAGGGAACAAAAGCTGGAGCTGCAAGCTTAATGTAGTCTTA
TGCAATACTCTTGTAGTCTTGCAACATGGTAACGATGAGTTAGCAACATGCCTTACAAGGAGAGAAAAAGCACCGTGCATGCCGA
TTGGTGGAAGTAAGGTGGTACGATCGTGCCTTATTAGGAAGGCAACAGACGGGTCTGACATGGATTGGACGAACCACTGAATTGC
CGCATTGCAGAGATATTGTATTTAAGTGCCTAGCTCGATACATAAACGGGTCTCTCTGGTTAGACCAGATCTGAGCCTGGGAGCTC
TCTGGCTAACTAGGGAACCCACTGCTTAAGCCTCAATAAAGCTTGCCTTGAGTGCTTCAAGTAGTGTGTGCCCGTCTGTTGTGTGA
CTCTGGTAACTAGAGATCCCTCAGACCCTTTTAGTCAGTGTGGAAAATCTCTAGCAGTGGCGCCCGAACAGGGACTTGAAAGCGA
AAGGGAAACCAGAGGAGCTCTCTCGACGCAGGACTCGGCTTGCTGAAGCGCGCACGGCAAGAGGCGAGGGGCGGCGACTGGTG
AGTACGCCAAAAATTTTGACTAGCGGAGGCTAGAAGGAGAGAGATGGGTGCGAGAGCGTCAGTATTAAGCGGGGGAGAATTAGA
TCGCGATGGGAAAAAATTCGGTTAAGGCCAGGGGGAAAGAAAAAATATAAATTAAAACATATAGTATGGGCAAGCAGGGAGCTA
GAACGATTCGCAGTTAATCCTGGCCTGTTAGAAACATCAGAAGGCTGTAGACAAATACTGGGACAGCTACAACCATCCCTTCAGA
CAGGATCAGAAGAACTTAGATCATTATATAATACAGTAGCAACCCTCTATTGTGTGCATCAAAGGATAGAGATAAAAGACACCAA
GGAAGCTTTAGACAAGATAGAGGAAGAGCAAAACAAAAGTAAGACCACCGCACAGCAAGCGGCCGCTGATCTTCAGACCTGGA
GGAGGAGATATGAGGGACAATTGGAGAAGTGAATTATATAAATATAAAGTAGTAAAAATTGAACCATTAGGAGTAGCACCCACC
AAGGCAAAGAGAAGAGTGGTGCAGAGAGAAAAAAGAGCAGTGGGAATAGGAGCTTTGTTCCTTGGGTTCTTGGGAGCAGCAGGA
AGCACTATGGGCGCAGCGTCAATGACGCTGACGGTACAGGCCAGACAATTATTGTCTGGTATAGTGCAGCAGCAGAACAATTTGC
TGAGGGCTATTGAGGCGCAACAGCATCTGTTGCAACTCACAGTCTGGGGCATCAAGCAGCTCCAGGCAAGAATCCTGGCTGTGGA
AAGATACCTAAAGGATCAACAGCTCCTGGGGATTTGGGGTTGCTCTGGAAAACTCATTTGCACCACTGCTGTGCCTTGGAATGCTA
GTTGGAGTAATAAATCTCTGGAACAGATTTGGAATCACACGACCTGGATGGAGTGGGACAGAGAAATTAACAATTACACAAGCTT
AATACACTCCTTAATTGAAGAATCGCAAAACCAGCAAGAAAAGAATGAACAAGAATTATTGGAATTAGATAAATGGGCAAGTTT
GTGGAATTGGTTTAACATAACAAATTGGCTGTGGTATATAAAATTATTCATAATGATAGTAGGAGGCTTGGTAGGTTTAAGAATA
GTTTTTGCTGTACTTTCTATAGTGAATAGAGTTAGGCAGGGATATTCACCATTATCGTTTCAGACCCACCTCCCAACCCCGAGGGG
ACCCTTGCGCCTTTTCCAAGGCAGCCCTGGGTTTGCGCAGGGACGCGGCTGCTCTGGGCGTGGTTCCGGGAAACGCAGCGGCGCC
GACCCTGGGTCTCGCACATTCTTCACGTCCGTTCGCAGCGTCACCCGGATCTTCGCCGCTACCCTTGTGGGCCCCCCGGCGACGCT
TCCTGCTCCGCCCCTAAGTCGGGAAGGTTCCTTGCGGTTCGCGGCGTGCCGGACGTGACAAACGGAAGCCGCACGACTCACTAGT
ACCCTCGCAGACGGACAGCGCCAGGGAGCAATGGCAGCGCGCCGACCGCGATGGGCTGTGGCCAATAGCGGCTGCTCAGCAGGG
CGCGCCGAGAGCAGCGGCCGGGAAGGGGCGGTGCGGGAGGCGGGGTGTGGGGCGGTAGTGTGGGCCCTGTTCCTGCCCGCGCGG
TGTTCCGCATTCTGCAAGCCTCCGGAGCGCACGTCGGCAGTCGGCTCCCTCGTTGACCGAATCACCGACCTCTCTCCCCAGGGATC
GATACCGTCGACTCCGGAATAGCCACCATGGTGAGCAAGGGCGAGGAGCTGTTCACCGGGGTGGTGCCCATCCTGGTCGAGCTGG
ACGGCGACGTAAACGGCCACAAGTTCAGCGTGTCCGGCGAGGGCGAGGGCGATGCCACCTACGGCAAGCTGACCCTGAAGTTCA
TCTGCACCACCGGCAAGCTGCCCGTGCCCTGGCCCACCCTCGTGACCACCCTGACCTACGGCGTGCAGTGCTTCAGCCGCTACCCC
GACCACATGAAGCAGCACGACTTCTTCAAGTCCGCCATGCCCGAAGGCTACGTCCAGGAGCGCACCATCTTCTTCAAGGACGACG
GCAACTACAAGACCCGCGCCGAGGTGAAGTTCGAGGGCGACACCCTGGTGAACCGCATCGAGCTGAAGGGCATCGACTTCAAGG
AGGACGGCAACATCCTGGGGCACAAGCTGGAGTACAACTACAACAGCCACAACGTCTATATCATGGCCGACAAGCAGAAGAACG
GCATCAAGGTGAACTTCAAGATCCGCCACAACATCGAGGACGGCAGCGTGCAGCTCGCCGACCACTACCAGCAGAACACCCCCA
TCGGCGACGGCCCCGTGCTGCTGCCCGACAACCACTACCTGAGCACCCAGTCCGCCCTGAGCAAAGACCCCAACGAGAAGCGCG
ATCACATGGTCCTGCTGGAGTTCGTGACCGCCGCCGGGATCACTCTCGGCATGGACGAGCTGTACAAGGAGACGGACGTCTCATA
AATGCATGAGTAACTGAGGATCCGCCCCTCTCCCTCCCCCCCCCCTAACGTTACTGGCCGAAGCCGCTTGGAATAAGGCCGGTGT
GCGTTTGTCTATATGTTATTTTCCACCATATTGCCGTCTTTTGGCAATGTGAGGGCCCGGAAACCTGGCCCTGTCTTCTTGACGAGC
ATTCCTAGGGGTCTTTCCCCTCTCGCCAAAGGAATGCAAGGTCTGTTGAATGTCGTGAAGGAAGCAGTTCCTCTGGAAGCTTCTTG
AAGACAAACAACGTCTGTAGCGACCCTTTGCAGGCAGCGGAACCCCCCACCTGGCGACAGGTGCCTCTGCGGCCAAAAGCCACG
TGTATAAGATACACCTGCAAAGGCGGCACAACCCCAGTGCCACGTTGTGAGTTGGATAGTTGTGGAAAGAGTCAAATGGCTCTCC
TCAAGCGTATTCAACAAGGGGCTGAAGGATGCCCAGAAGGTACCCCATTGTATGGGATCTGATCTGGGGCCTCGGTACACATGCT
TTACATGTGTTTAGTCGAGGTTAAAAAAACGTCTAGGCCCCCCGAACCACGGGGACGTGGTTTTCCTTTGAAAAACACGATGATA
ATATGGCCACAACCCTGGAATTCGCCACCATGGTGAGCAAGGGCGAGGAGGATAACATGGCCATCATCAAGGAGTTCATGCGCTT
CAAGGTGCACATGGAGGGCTCCGTGAACGGCCACGAGTTCGAGATCGAGGGCGAGGGCGAGGGCCGCCCCTACGAGGGCACCCA
GACCGCCAAGCTGAAGGTGACCAAGGGTGGCCCCCTGCCCTTCGCCTGGGACATCCTGTCCCCTCAGTTCATGTACGGCTCCAAG
GCCTACGTGAAGCACCCCGCCGACATCCCCGACTACTTGAAGCTGTCCTTCCCCGAGGGCTTCAAGTGGGAGCGCGTGATGAACT
TCGAGGACGGCGGCGTGGTGACCGTGACCCAGGACTCCTCCCTGCAGGACGGCGAGTTCATCTACAAGGTGAAGCTGCGCGGCA
CCAACTTCCCCTCCGACGGCCCCGTAATGCAGAAGAAGACCATGGGCTGGGAGGCCTCCTCCGAGCGGATGTACCCCGAGGACG
GCGCCCTGAAGGGCGAGATCAAGCAGAGGCTGAAGCTGAAGGACGGCGGCCACTACGACGCTGAGGTCAAGACCACCTACAAG
GCCAAGAAGCCCGTGCAGCTGCCCGGCGCCTACAACGTCAACATCAAGTTGGACATCACCTCCCACAACGAGGACTACACCATCG
TGGAACAGTACGAACGCGCCGAGGGCCGCCACTCCACCGGCGGCATGGACGAGCTGTACAAGTAAACTAGTAAGCTTGGCGTAA
CTAGATCTTGAGACAAATGGCAGTATTCATCCACAATTTTAAAAGAAAAGGGGGGATTGGGGGGTACAGTGCAGGGGAAAGAAT
AGTAGACATAATAGCAACAGACATACAAACTAAAGAATTACAAAAACAAATTACAAAAATTCAAAATTTTCGGGTTTATTACAGG
GACAGCAGAGATCCACTTTGGGCTCGAGGGGGCCCGGGTGCAAAGATGGATAAAGTTTTAAACAGAGAGGAATCTTTGCAGCTA
ATGGACCTTCTAGGTCTTGAAAGGAGTGGGAATTGGCTCCGGTGCCCGTCAGTGGGCAGAGCGCACATCGCCCACAGTCCCCGAG
AAGTTGGGGGGAGGGGTCGGCAATTGATCCGGTGCCTAGAGAAGGTGGCGCGGGGTAAACTGGGAAAGTGATGTCGTGTACTGG
CTCCGCCTTTTTCCCGAGGGTGGGGGAGAACCGTATATAAGTGCAGTAGTCGCCGTGAACGTTCTTTTTCGCAACGGGTTTGCCGC
CAGAACACAGGTAAGTGCCGTGTGTGGTTCCCGCGGGCCTGGCCTCTTTACGGGTTATGGCCCTTGCGTGCCTTGAATTACTTCCA
CCTGGCTGCAGTACGTGATTCTTGATCCCGAGCTTCGGGTTGGAAGTGGGTGGGAGAGTTCGAGGCCTTGCGCTTAAGGAGCCCC
TTCGCCTCGTGCTTGAGTTGAGGCCTGGCCTGGGCGCTGGGGCCGCCGCGTGCGAATCTGGTGGCACCTTCGCGCCTGTCTCGCTG
CTTTCGATAAGTCTCTAGCCATTTAAAATTTTTGATGACCTGCTGCGACGCTTTTTTTCTGGCAAGATAGTCTTGTAAATGCGGGCC
AAGATCTGCACACTGGTATTTCGGTTTTTGGGGCCGCGGGCGGCGACGGGGCCCGTGCGTCCCAGCGCACATGTTCGGCGAGGCG
GGGCCTGCGAGCGCGGCCACCGAGAATCGGACGGGGGTAGTCTCAAGCTGGCCGGCCTGCTCTGGTGCCTGGCCTCGCGCCGCCG
TGTATCGCCCCGCCCTGGGCGGCAAGGCTGGCCCGGTCGGCACCAGTTGCGTGAGCGGAAAGATGGCCGCTTCCCGGCCCTGCTG
CAGGGAGCTCAAAATGGAGGACGCGGCGCTCGGGAGAGCGGGGGGTGAGTCACCCACACAAAGGAAAAGGGCCTTTCCGTCCT
CAGCCGTCGCTTCATGTGACTCCACGGAGTACCGGGCGCCGTCCAGGCACCTCGATTAGTTCTCGAGCTTTTGGAGTACGTCGTCT
TTAGGTTGGGGGGAGGGGTTTTATGCGATGGAGTTTCCCCACACTGAGTGGGTGGAGACTGAAGTTAGGCCAGCTTGGCACTTGA
TGTAATTCTCCTTGGAATTTGCCCTTTTTGAGTTTGGATCTTGGTTCATTCTCAAGCCTCAGACAGTGGTTCAAAGTTTTTTTCTTCC
ATTTCAGGTGTCGTGACGTACGGCCACCATGACCGAGTACAAGCCCACGGTGCGCCTCGCCACCCGCGACGACGTCCCCAGGGCC
GTACGCACCCTCGCCGCCGCGTTCGCCGACTACCCCGCCACGCGCCACACCGTCGATCCGGACCGCCACATCGAGCGGGTCACCG
AGCTGCAAGAACTCTTCCTCACGCGCGTCGGGCTCGACATCGGCAAGGTGTGGGTCGCGGACGACGGCGCCGCCGTGGCGGTCTG
GACCACGCCGGAGAGCGTCGAAGCGGGGGCGGTGTTCGCCGAGATCGGCCCGCGCATGGCCGAGTTGAGCGGTTCCCGGCTGGC
CGCGCAGCAACAGATGGAAGGCCTCCTGGCGCCGCACCGGCCCAAGGAGCCCGCGTGGTTCCTGGCCACCGTCGGAGTCTCGCCC
GACCACCAGGGCAAGGGTCTGGGCAGCGCCGTCGTGCTCCCCGGAGTGGAGGCGGCCGAGCGCGCCGGGGTGCCCGCCTTCCTG
GAGACCTCCGCGCCCCGCAACCTCCCCTTCTACGAGCGGCTCGGCTTCACCGTCACCGCCGACGTCGAGGTGCCCGAAGGACCGC
GCACCTGGTGCATGACCCGCAAGCCCGGTGCCTGAACGCGTTAAGTCGACAATCAACCTCTGGATTACAAAATTTGTGAAAGATT
GACTGGTATTCTTAACTATGTTGCTCCTTTTACGCTATGTGGATACGCTGCTTTAATGCCTTTGTATCATGCTATTGCTTCCCGTATG
GCTTTCATTTTCTCCTCCTTGTATAAATCCTGGTTGCTGTCTCTTTATGAGGAGTTGTGGCCCGTTGTCAGGCAACGTGGCGTGGTG
TGCACTGTGTTTGCTGACGCAACCCCCACTGGTTGGGGCATTGCCACCACCTGTCAGCTCCTTTCCGGGACTTTCGCTTTCCCCCTC
CCTATTGCCACGGCGGAACTCATCGCCGCCTGCCTTGCCCGCTGCTGGACAGGGGCTCGGCTGTTGGGCACTGACAATTCCGTGGT
GTTGTCGGGGAAATCATCGTCCTTTCCTTGGCTGCTCGCCTGTGTTGCCACCTGGATTCTGCGCGGGACGTCCTTCTGCTACGTCCC
TTCGGCCCTCAATCCAGCGGACCTTCCTTCCCGCGGCCTGCTGCCGGCTCTGCGGCCTCTTCCGCGTCTTCGCCTTCGCCCTCAGAC
GAGTCGGATCTCCCTTTGGGCCGCCTCCCCGCGTCGACTTTAAGACCAATGACTTACAAGGCAGCTGTAGATCTTAGCCACTTTTT
AAAAGAAAAGGGGGGACTGGAAGGGCTAATTCACTCCCAACGAAGACAAGATCTGCTTTTTGCTTGTACTGGGTCTCTCTGGTTA
GACCAGATCTGAGCCTGGGAGCTCTCTGGCTAACTAGGGAACCCACTGCTTAAGCCTCAATAAAGCTTGCCTTGAGTGCTTCAAGT
AGTGTGTGCCCGTCTGTTGTGTGACTCTGGTAACTAGAGATCCCTCAGACCCTTTTAGTCAGTGTGGAAAATCTCTAGCAGTACGT
ATAGTAGTTCATGTCATCTTATTATTCAGTATTTATAACTTGCAAAGAAATGAATATCAGAGAGTGAGAGGAACTTGTTTATTGCA
GCTTATAATGGTTACAAATAAAGCAATAGCATCACAAATTTCACAAATAAAGCATTTTTTTCACTGCATTCTAGTTGTGGTTTGTC
CAAACTCATCAATGTATCTTATCATGTCTGGCTCTAGCTATCCCGCCCCTAACTCCGCCCATCCCGCCCCTAACTCCGCCCAGTTCC
GCCCATTCTCCGCCCCATGGCTGACTAATTTTTTTTATTTATGCAGAGGCCGAGGCCGCCTCGGCCTCTGAGCTATTCCAGAAGTA
GTGAGGAGGCTTTTTTGGAGGCCTAGGGACGTACCCAATTCGCCCTATAGTGAGTCGTATTACGCGCGCTCACTGGCCGTCGTTTT
ACAACGTCGTGACTGGGAAAACCCTGGCGTTACCCAACTTAATCGCCTTGCAGCACATCCCCCTTTCGCCAGCTGGCGTAATAGC
GAAGAGGCCCGCACCGATCGCCCTTCCCAACAGTTGCGCAGCCTGAATGGCGAATGGGACGCGCCCTGTAGCGGCGCATTAAGC
GCGGCGGGTGTGGTGGTTACGCGCAGCGTGACCGCTACACTTGCCAGCGCCCTAGCGCCCGCTCCTTTCGCTTTCTTCCCTTCCTTT
CTCGCCACGTTCGCCGGCTTTCCCCGTCAAGCTCTAAATCGGGGGCTCCCTTTAGGGTTCCGATTTAGTGCTTTACGGCACCTCGA
CCCCAAAAAACTTGATTAGGGTGATGGTTCACGTAGTGGGCCATCGCCCTGATAGACGGTTTTTCGCCCTTTGACGTTGGAGTCCA
CGTTCTTTAATAGTGGACTCTTGTTCCAAACTGGAACAACACTCAACCCTATCTCGGTCTATTCTTTTGATTTATAAGGGATTTTGC
CGATTTCGGCCTATTGGTTAAAAAATGAGCTGATTTAACAAAAATTTAACGCGAATTTTAACAAAATATTAACGCTTACAATTTAG
GTGGCACTTTTCGGGGAAATGTGCGCGGAACCCCTATTTGTTTATTTTTCTAAATACATTCAAATATGTATCCGCTCATGAGACAA
TAACCCTGATAAATGCTTCAATAATATTGAAAAAGGAAGAGTATGAGTATTCAACATTTCCGTGTCGCCCTTATTCCCTTTTTTGC
GGCATTTTGCCTTCCTGTTTTTGCTCACCCAGAAACGCTGGTGAAAGTAAAAGATGCTGAAGATCAGTTGGGTGCACGAGTGGGTT
ACATCGAACTGGATCTCAACAGCGGTAAGATCCTTGAGAGTTTTCGCCCCGAAGAACGTTTTCCAATGATGAGCACTTTTAAAGTT
CTGCTATGTGGCGCGGTATTATCCCGTATTGACGCCGGGCAAGAGCAACTCGGTCGCCGCATACACTATTCTCAGAATGACTTGGT
TGAGTACTCACCAGTCACAGAAAAGCATCTTACGGATGGCATGACAGTAAGAGAATTATGCAGTGCTGCCATAACCATGAGTGAT
AACACTGCGGCCAACTTACTTCTGACAACGATCGGAGGACCGAAGGAGCTAACCGCTTTTTTGCACAACATGGGGGATCATGTAA
CTCGCCTTGATCGTTGGGAACCGGAGCTGAATGAAGCCATACCAAACGACGAGCGTGACACCACGATGCCTGTAGCAATGGCAA
CAACGTTGCGCAAACTATTAACTGGCGAACTACTTACTCTAGCTTCCCGGCAACAATTAATAGACTGGATGGAGGCGGATAAAGT
TGCAGGACCACTTCTGCGCTCGGCCCTTCCGGCTGGCTGGTTTATTGCTGATAAATCTGGAGCCGGTGAGCGTGGGTCTCGCGGTA
TCATTGCAGCACTGGGGCCAGATGGTAAGCCCTCCCGTATCGTAGTTATCTACACGACGGGGAGTCAGGCAACTATGGATGAACG
AAATAGACAGATCGCTGAGATAGGTGCCTCACTGATTAAGCATTGGTAACTGTCAGACCAAGTTTACTCATATATACTTTAGATTG
ATTTAAAACTTCATTTTTAATTTAAAAGGATCTAGGTGAAGATCCTTTTTGATAATCTCATGACCAAAATCCCTTAACGTGAGTTTT
CGTTCCACTGAGCGTCAGACCCCGTAGAAAAGATCAAAGGATCTTCTTGAGATCCTTTTTTTCTGCGCGTAATCTGCTGCTTGCAA
ACAAAAAAACCACCGCTACCAGCGGTGGTTTGTTTGCCGGATCAAGAGCTACCAACTCTTTTTCCGAAGGTAACTGGCTTCAGCA
GAGCGCAGATACCAAATACTGTTCTTCTAGTGTAGCCGTAGTTAGGCCACCACTTCAAGAACTCTGTAGCACCGCCTACATACCTC
GCTCTGCTAATCCTGTTACCAGTGGCTGCTGCCAGTGGCGATAAGTCGTGTCTTACCGGGTTGGACTCAAGACGATAGTTACCGGA
TAAGGCGCAGCGGTCGGGCTGAACGGGGGGTTCGTGCACACAGCCCAGCTTGGAGCGAACGACCTACACCGAACTGAGATACCT
ACAGCGTGAGCTATGAGAAAGCGCCACGCTTCCCGAAGGGAGAAAGGCGGACAGGTATCCGGTAAGCGGCAGGGTCGGAACAG
GAGAGCGCACGAGGGAGCTTCCAGGGGGAAACGCCTGGTATCTTTATAGTCCTGTCGGGTTTCGCCACCTCTGACTTGAGCGTCG
ATTTTTGTGATGCTCGTCAGGGGGGCGGAGCCTATGGAAAAACGCCAGCAACGCGGCCTTTTTACGGTTCCTGGCCTTTTGCTGGC
CTTTTGCTCACATGTTCTTTCCTGCGTTATCCCCTGATTCTGTGGATAACCGTATTACCGCCTTTGAGTGAGCTGATACCGCTCGCC
GCAGC
pJL002
(SEQ ID NO: 305)
GTCGACGGATCGGGAGATCTCCCGATCCCCTATGGTGCACTCTCAGTACAATCTGCTCTGATGCCGCATAGTTAAGCCAGTATCTG
CTCCCTGCTTGTGTGTTGGAGGTCGCTGAGTAGTGCGCGAGCAAAATTTAAGCTACAACAAGGCAAGGCTTGACCGACAATTGCA
TGAAGAATCTGCTTAGGGTTAGGCGTTTTGCGCTGCTTCGCGATGTACGGGCCAGATATACGCGTTGACATTGATTATTGACTAGT
TATTAATAGTAATCAATTACGGGGTCATTAGTTCATAGCCCATATATGGAGTTCCGCGTTACATAACTTACGGTAAATGGCCCGCC
TGGCTGACCGCCCAACGACCCCCGCCCATTGACGTCAATAATGACGTATGTTCCCATAGTAACGCCAATAGGGACTTTCCATTGAC
GTCAATGGGTGGAGTATTTACGGTAAACTGCCCACTTGGCAGTACATCAAGTGTATCATATGCCAAGTACGCCCCCTATTGACGTC
AATGACGGTAAATGGCCCGCCTGGCATTATGCCCAGTACATGACCTTATGGGACTTTCCTACTTGGCAGTACATCTACGTATTAGT
CATCGCTATTACCATGGTGATGCGGTTTTGGCAGTACATCAATGGGCGTGGATAGCGGTTTGACTCACGGGGATTTCCAAGTCTCC
ACCCCATTGACGTCAATGGGAGTTTGTTTTGGCACCAAAATCAACGGGACTTTCCAAAATGTCGTAACAACTCCGCCCCATTGACG
CAAATGGGCGGTAGGCGTGTACGGTGGGAGGTCTATATAAGCAGCGCGTTTTGCCTGTACTGGGTCTCTCTGGTTAGACCAGATCT
GAGCCTGGGAGCTCTCTGGCTAACTAGGGAACCCACTGCTTAAGCCTCAATAAAGCTTGCCTTGAGTGCTTCAAGTAGTGTGTGCC
CGTCTGTTGTGTGACTCTGGTAACTAGAGATCCCTCAGACCCTTTTAGTCAGTGTGGAAAATCTCTAGCAGTGGCGCCCGAACAGG
GACTTGAAAGCGAAAGGGAAACCAGAGGAGCTCTCTCGACGCAGGACTCGGCTTGCTGAAGCGCGCACGGCAAGAGGCGAGGG
GCGGCGACTGGTGAGTACGCCAAAAATTTTGACTAGCGGAGGCTAGAAGGAGAGAGATGGGTGCGAGAGCGTCAGTATTAAGCG
GGGGAGAATTAGATCGCGATGGGAAAAAATTCGGTTAAGGCCAGGGGGAAAGAAAAAATATAAATTAAAACATATAGTATGGGC
AAGCAGGGAGCTAGAACGATTCGCAGTTAATCCTGGCCTGTTAGAAACATCAGAAGGCTGTAGACAAATACTGGGACAGCTACA
ACCATCCCTTCAGACAGGATCAGAAGAACTTAGATCATTATATAATACAGTAGCAACCCTCTATTGTGTGCATCAAAGGATAGAG
ATAAAAGACACCAAGGAAGCTTTAGACAAGATAGAGGAAGAGCAAAACAAAAGTAAGACCACCGCACAGCAAGCGGCCGGCCG
CGCTGATCTTCAGACCTGGAGGAGGAGATATGAGGGACAATTGGAGAAGTGAATTATATAAATATAAAGTAGTAAAAATTGAAC
CATTAGGAGTAGCACCCACCAAGGCAAAGAGAAGAGTGGTGCAGAGAGAAAAAAGAGCAGTGGGAATAGGAGCTTTGTTCCTTG
GGTTCTTGGGAGCAGCAGGAAGCACTATGGGCGCAGCGTCAATGACGCTGACGGTACAGGCCAGACAATTATTGTCTGGTATAGT
GCAGCAGCAGAACAATTTGCTGAGGGCTATTGAGGCGCAACAGCATCTGTTGCAACTCACAGTCTGGGGCATCAAGCAGCTCCAG
GCAAGAATCCTGGCTGTGGAAAGATACCTAAAGGATCAACAGCTCCTGGGGATTTGGGGTTGCTCTGGAAAACTCATTTGCACCA
CTGCTGTGCCTTGGAATGCTAGTTGGAGTAATAAATCTCTGGAACAGATTTGGAATCACACGACCTGGATGGAGTGGGACAGAGA
AATTAACAATTACACAAGCTTAATACACTCCTTAATTGAAGAATCGCAAAACCAGCAAGAAAAGAATGAACAAGAATTATTGGA
ATTAGATAAATGGGCAAGTTTGTGGAATTGGTTTAACATAACAAATTGGCTGTGGTATATAAAATTATTCATAATGATAGTAGGA
GGCTTGGTAGGTTTAAGAATAGTTTTTGCTGTACTTTCTATAGTGAATAGAGTTAGGCAGGGATATTCACCATTATCGTTTCAGAC
CCACCTCCCAACCCCGAGGGGACCCGACAGGCCCGAAGGAATAGAAGAAGAAGGTGGAGAGAGAGACAGAGACAGATCCATTC
GATTAGTGAACGGATCGGCACTGCGTGCGCCAATTCTGCAGACAAATGGCAGTATTCATCCACAATTTTAAAAGAAAAGGGGGGA
TTGGGGGGTACAGTGCAGGGGAAAGAATAGTAGACATAATAGCAACAGACATACAAACTAAAGAATTACAAAAACAAATTACAA
AAATTCAAAATTTTCGGGTTTATTACAGGGACAGCAGAGATCCAGTTTGGTTAGTACCGGGCCCGCTCTAGCCGTGAGGCTCCGGT
GCCCGTCAGTGGGCAGAGCGCACATCGCCCACAGTCCCCGAGAAGTTGGGGGGAGGGGTCGGCAATTGAACCGGTGCCTAGAGA
AGGTGGCGCGGGGTAAACTGGGAAAGTGATGTCGTGTACTGGCTCCGCCTTTTTCCCGAGGGTGGGGGAGAACCGTATATAAGTG
CAGTAGTCGCCGTGAACGTTCTTTTTCGCAACGGGTTTGCCGCCAGAACACAGGTAAGTGCCGTGTGTGGTTCCCGCGGGCCTGGC
CTCTTTACGGGTTATGGCCCTTGCGTGCCTTGAATTACTTCCACCTGGCTGCAGTACGTGATTCTTGATCCCGAGCTTCGGGTTGGA
AGTGGGTGGGAGAGTTCGAGGCCTTGCGCTTAAGGAGCCCCTTCGCCTCGTGCTTGAGTTGAGGCCTGGCCTGGGCGCTGGGGCC
GCCGCGTGCGAATCTGGTGGCACCTTCGCGCCTGTCTCGCTGCTTTCGATAAGTCTCTAGCCATTTAAAATTTTTGATGACCTGCTG
CGACGCTTTTTTTCTGGCAAGATAGTCTTGTAAATGCGGGCCAAGATCTGCACACTGGTATTTCGGTTTTTGGGGCCGCGGGCGGC
GACGGGGCCCGTGCGTCCCAGCGCACATGTTCGGCGAGGCGGGGCCTGCGAGCGCGGCCACCGAGAATCGGACGGGGGTAGTCT
CAAGCTGGCCGGCCTGCTCTGGTGCCTGGCCTCGCGCCGCCGTGTATCGCCCCGCCCTGGGCGGCAAGGCTGGCCCGGTCGGCAC
CAGTTGCGTGAGCGGAAAGATGGCCGCTTCCCGGCCCTGCTGCAGGGAGCTCAAAATGGAGGACGCGGCGCTCGGGAGAGCGGG
CGGGTGAGTCACCCACACAAAGGAAAAGGGCCTTTCCGTCCTCAGCCGTCGCTTCATGTGACTCCACGGAGTACCGGGCGCCGTC
CAGGCACCTCGATTAGTTCTCGAGCTTTTGGAGTACGTCGTCTTTAGGTTGGGGGGAGGGGTTTTATGCGATGGAGTTTCCCCACA
CTGAGTGGGTGGAGACTGAAGTTAGGCCAGCTTGGCACTTGATGTAATTCTCCTTGGAATTTGCCCTTTTTGAGTTTGGATCTTGGT
TCATTCTCAAGCCTCAGACAGTGGTTCAAAGTTTTTTTCTTCCATTTCAGGTGTCGTGAGCGGCCGCTGAGTTAACTATTCTAGAGC
CACCATGGAGACGGACGTCTCAGGTTCAGGTGCAACGAACTTCTCATTGTTGAAGCAAGCCGGTGATGTTGAGGAAAATCCGGGT
CCTATGGTGTCTAAGGGCGAAGAGCTGATTAAGGAGAACATGCACATGAAGCTGTACATGGAGGGCACCGTGGACAACCATCAC
TTCAAGTGCACATCCGAGGGCGAAGGCAAGCCCTACGAGGGCACCCAGACCATGAGAATCAAGGTGGTCGAGGGCGGCCCTCTC
CCCTTCGCCTTCGACATCCTGGCTACTAGCTTCCTCTACGGCAGCAAGACCTTCATCAACCACACCCAGGGCATCCCCGACTTCTT
CAAGCAGTCCTTCCCTGAGGGCTTCACATGGGAGAGAGTCACCACATACGAAGACGGGGGCGTGCTGACCGCTACCCAGGACAC
CAGCCTCCAGGACGGCTGCCTCATCTACAACGTCAAGATCAGAGGGGTGAACTTCACATCCAACGGCCCTGTGATGCAGAAGAAA
ACACTCGGCTGGGAGGCCTTCACCGAGACCCTGTACCCCGCTGACGGCGGCCTGGAAGGCAGAAACGACATGGCCCTGAAGCTC
GTGGGCGGGAGCCATCTGATCGCAAACGCCAAGACCACATATAGATCCAAGAAACCCGCTAAGAACCTCAAGATGCCTGGCGTC
TACTATGTGGACTACAGACTGGAAAGAATCAAGGAGGCCAACAACGAGACCTACGTCGAGCAGCACGAGGTGGCAGTGGCCAGA
TACTGCGACCTCCCTAGCAAACTGGGGCACAAGCTTAATTAAAAGTAAacgegtctggaacaatcaacctctggattacaaaatttgtgaaagattga
ctggtattcttaactatgttgctccttttacgctatgtggatacgctgctttaatgcctttgtatcatgctattgcttcccgtatggctttcattttc
tcctccttgtataaatcctggttgctgtctctttatgaggagttgtggcccgttgtcaggcaacgtggcgtggtgtgcactgtgtttgctgacgcaac
ccccactggttggggcattgccaccacctgtcagctcctttccgggactttcgctttccccctccctattgccacggcggaactcatcgccgcctgcc
ttgcccgctgctggacaggggcteggctgttgggcactgacaattccgtggtgttgtcggggaagctgacgtectttccatggctgctcgcctgtgtt
gccacctggattctgcgcgggacgtccttctgctacgtcccttcggccctcaatccagcggaccttccttcccgcggcctgctgccggctctgcggcc
tcttccgcgtcttcgccttcgccctcagacgagtcggatctccctttgggccgcctccccgcctgaagtaaAGCGGCCGCATCG
ATACCGTCGACCTCGATCGAGACCTAGAAAAACATGGAGCAATCACAAGTAGCAATACAGCAGCTACCAATGCTGATTGTGCCTG
GCTAGAAGCACAAGAGGAGGAGGAGGTGGGTTTTCCAGTCACACCTCAGGTACCTTTAAGACCAATGACTTACAAGGCAGCTGTA
GATCTTAGCCACTTTTTAAAAGAAAAGGGGGGACTGGAAGGGCTAATTCACTCCCAACGAAGACAAGATATCCTTGATCTGTGGA
TCTACCACACACAAGGCTACTTCCCTGATTGGCAGAACTACACACCAGGGCCAGGGATCAGATATCCACTGACCTTTGGATGGTG
CTACAAGCTAGTACCAGTTGAGCAAGAGAAGGTAGAAGAAGCCAATGAAGGAGAGAACACCCGCTTGTTACACCCTGTGAGCCT
GCATGGGATGGATGACCCGGAGAGAGAAGTATTAGAGTGGAGGTTTGACAGCCGCCTAGCATTTCATCACATGGCCCGAGAGCT
GCATCCGGACTGTACTGGGTCTCTCTGGTTAGACCAGATCTGAGCCTGGGAGCTCTCTGGCTAACTAGGGAACCCACTGCTTAAGC
CTCAATAAAGCTTGCCTTGAGTGCTTCAAGTAGTGTGTGCCCGTCTGTTGTGTGACTCTGGTAACTAGAGATCCCTCAGACCCTTTT
AGTCAGTGTGGAAAATCTCTAGCAGCATGTGAGCAAAAGGCCAGCAAAAGGCCAGGAACCGTAAAAAGGCCGCGTTGCTGGCGT
TTTTCCATAGGCTCCGCCCCCCTGACGAGCATCACAAAAATCGACGCTCAAGTCAGAGGTGGCGAAACCCGACAGGACTATAAAG
ATACCAGGCGTTTCCCCCTGGAAGCTCCCTCGTGCGCTCTCCTGTTCCGACCCTGCCGCTTACCGGATACCTGTCCGCCTTTCTCCC
TTCGGGAAGCGTGGCGCTTTCTCATAGCTCACGCTGTAGGTATCTCAGTTCGGTGTAGGTCGTTCGCTCCAAGCTGGGCTGTGTGC
ACGAACCCCCCGTTCAGCCCGACCGCTGCGCCTTATCCGGTAACTATCGTCTTGAGTCCAACCCGGTAAGACACGACTTATCGCCA
CTGGCAGCAGCCACTGGTAACAGGATTAGCAGAGCGAGGTATGTAGGCGGTGCTACAGAGTTCTTGAAGTGGTGGCCTAACTACG
GCTACACTAGAAGAACAGTATTTGGTATCTGCGCTCTGCTGAAGCCAGTTACCTTCGGAAAAAGAGTTGGTAGCTCTTGATCCGGC
AAACAAACCACCGCTGGTAGCGGTGGTTTTTTTGTTTGCAAGCAGCAGATTACGCGCAGAAAAAAAGGATCTCAAGAAGATCCTT
TGATCTTTTCTACGGGGTCTGACGCTCAGTGGAACGAAAACTCACGTTAAGGGATTTTGGTCATGAGATTATCAAAAAGGATCTTC
ACCTAGATCCTTTTAAATTAAAAATGAAGTTTTAAATCAATCTAAAGTATATATGAGTAAACTTGGTCTGACAGTTACCAATGCTT
AATCAGTGAGGCACCTATCTCAGCGATCTGTCTATTTCGTTCATCCATAGTTGCCTGACTCCCCGTCGTGTAGATAACTACGATAC
GGGAGGGCTTACCATCTGGCCCCAGTGCTGCAATGATACCGCGAGACCCACGCTCACCGGCTCCAGATTTATCAGCAATAAACCA
GCCAGCCGGAAGGGCCGAGCGCAGAAGTGGTCCTGCAACTTTATCCGCCTCCATCCAGTCTATTAATTGTTGCCGGGAAGCTAGA
GTAAGTAGTTCGCCAGTTAATAGTTTGCGCAACGTTGTTGCCATTGCTACAGGCATCGTGGTGTCACGCTCGTCGTTTGGTATGGC
TTCATTCAGCTCCGGTTCCCAACGATCAAGGCGAGTTACATGATCCCCCATGTTGTGCAAAAAAGCGGTTAGCTCCTTCGGTCCTC
CGATCGTTGTCAGAAGTAAGTTGGCCGCAGTGTTATCACTCATGGTTATGGCAGCACTGCATAATTCTCTTACTGTCATGCCATCC
GTAAGATGCTTTTCTGTGACTGGTGAGTACTCAACCAAGTCATTCTGAGAATAGTGTATGCGGCGACCGAGTTGCTCTTGCCCGGC
GTCAATACGGGATAATACCGCGCCACATAGCAGAACTTTAAAAGTGCTCATCATTGGAAAACGTTCTTCGGGGCGAAAACTCTCA
AGGATCTTACCGCTGTTGAGATCCAGTTCGATGTAACCCACTCGTGCACCCAACTGATCTTCAGCATCTTTTACTTTCACCAGCGTT
TCTGGGTGAGCAAAAACAGGAAGGCAAAATGCCGCAAAAAAGGGAATAAGGGCGACACGGAAATGTTGAATACTCATACTCTTC
CTTTTTCAATATTATTGAAGCATTTATCAGGGTTATTGTCTCATGAGCGGATACATATTTGAATGTATTTAGAAAAATAAACAAAT
AGGGGTTCCGCGCACATTTCCCCGAAAAGTGCCACCTGAC
pJL001
  (SEQ ID NO: 306)
GTCGACGGATCGGGAGATCTCCCGATCCCCTATGGTGCACTCTCAGTACAATCTGCTCTGATGCCGCATAGTTAAGCCAGTATCTG
CTCCCTGCTTGTGTGTTGGAGGTCGCTGAGTAGTGCGCGAGCAAAATTTAAGCTACAACAAGGCAAGGCTTGACCGACAATTGCA
TGAAGAATCTGCTTAGGGTTAGGCGTTTTGCGCTGCTTCGCGATGTACGGGCCAGATATACGCGTTGACATTGATTATTGACTAGT
TATTAATAGTAATCAATTACGGGGTCATTAGTTCATAGCCCATATATGGAGTTCCGCGTTACATAACTTACGGTAAATGGCCCGCC
TGGCTGACCGCCCAACGACCCCCGCCCATTGACGTCAATAATGACGTATGTTCCCATAGTAACGCCAATAGGGACTTTCCATTGAC
GTCAATGGGTGGAGTATTTACGGTAAACTGCCCACTTGGCAGTACATCAAGTGTATCATATGCCAAGTACGCCCCCTATTGACGTC
AATGACGGTAAATGGCCCGCCTGGCATTATGCCCAGTACATGACCTTATGGGACTTTCCTACTTGGCAGTACATCTACGTATTAGT
CATCGCTATTACCATGGTGATGCGGTTTTGGCAGTACATCAATGGGCGTGGATAGCGGTTTGACTCACGGGGATTTCCAAGTCTCC
ACCCCATTGACGTCAATGGGAGTTTGTTTTGGCACCAAAATCAACGGGACTTTCCAAAATGTCGTAACAACTCCGCCCCATTGACG
CAAATGGGCGGTAGGCGTGTACGGTGGGAGGTCTATATAAGCAGCGCGTTTTGCCTGTACTGGGTCTCTCTGGTTAGACCAGATCT
GAGCCTGGGAGCTCTCTGGCTAACTAGGGAACCCACTGCTTAAGCCTCAATAAAGCTTGCCTTGAGTGCTTCAAGTAGTGTGTGCC
CGTCTGTTGTGTGACTCTGGTAACTAGAGATCCCTCAGACCCTTTTAGTCAGTGTGGAAAATCTCTAGCAGTGGCGCCCGAACAGG
GACTTGAAAGCGAAAGGGAAACCAGAGGAGCTCTCTCGACGCAGGACTCGGCTTGCTGAAGCGCGCACGGCAAGAGGCGAGGG
GCGGCGACTGGTGAGTACGCCAAAAATTTTGACTAGCGGAGGCTAGAAGGAGAGAGATGGGTGCGAGAGCGTCAGTATTAAGCG
GGGGAGAATTAGATCGCGATGGGAAAAAATTCGGTTAAGGCCAGGGGGAAAGAAAAAATATAAATTAAAACATATAGTATGGGC
AAGCAGGGAGCTAGAACGATTCGCAGTTAATCCTGGCCTGTTAGAAACATCAGAAGGCTGTAGACAAATACTGGGACAGCTACA
ACCATCCCTTCAGACAGGATCAGAAGAACTTAGATCATTATATAATACAGTAGCAACCCTCTATTGTGTGCATCAAAGGATAGAG
ATAAAAGACACCAAGGAAGCTTTAGACAAGATAGAGGAAGAGCAAAACAAAAGTAAGACCACCGCACAGCAAGCGGCCGGCCG
CGCTGATCTTCAGACCTGGAGGAGGAGATATGAGGGACAATTGGAGAAGTGAATTATATAAATATAAAGTAGTAAAAATTGAAC
CATTAGGAGTAGCACCCACCAAGGCAAAGAGAAGAGTGGTGCAGAGAGAAAAAAGAGCAGTGGGAATAGGAGCTTTGTTCCTTG
GGTTCTTGGGAGCAGCAGGAAGCACTATGGGCGCAGCGTCAATGACGCTGACGGTACAGGCCAGACAATTATTGTCTGGTATAGT
GCAGCAGCAGAACAATTTGCTGAGGGCTATTGAGGCGCAACAGCATCTGTTGCAACTCACAGTCTGGGGCATCAAGCAGCTCCAG
GCAAGAATCCTGGCTGTGGAAAGATACCTAAAGGATCAACAGCTCCTGGGGATTTGGGGTTGCTCTGGAAAACTCATTTGCACCA
CTGCTGTGCCTTGGAATGCTAGTTGGAGTAATAAATCTCTGGAACAGATTTGGAATCACACGACCTGGATGGAGTGGGACAGAGA
AATTAACAATTACACAAGCTTAATACACTCCTTAATTGAAGAATCGCAAAACCAGCAAGAAAAGAATGAACAAGAATTATTGGA
ATTAGATAAATGGGCAAGTTTGTGGAATTGGTTTAACATAACAAATTGGCTGTGGTATATAAAATTATTCATAATGATAGTAGGA
GGCTTGGTAGGTTTAAGAATAGTTTTTGCTGTACTTTCTATAGTGAATAGAGTTAGGCAGGGATATTCACCATTATCGTTTCAGAC
CCACCTCCCAACCCCGAGGGGACCCGACAGGCCCGAAGGAATAGAAGAAGAAGGTGGAGAGAGAGACAGAGACAGATCCATTC
GATTAGTGAACGGATCGGCACTGCGTGCGCCAATTCTGCAGACAAATGGCAGTATTCATCCACAATTTTAAAAGAAAAGGGGGGA
TTGGGGGGTACAGTGCAGGGGAAAGAATAGTAGACATAATAGCAACAGACATACAAACTAAAGAATTACAAAAACAAATTACAA
AAATTCAAAATTTTCGGGTTTATTACAGGGACAGCAGAGATCCAGTTTGGTTAGTACCGGGCCCGCTCTAGCCGTGAGGCTCCGGT
GCCCGTCAGTGGGCAGAGCGCACATCGCCCACAGTCCCCGAGAAGTTGGGGGGAGGGGTCGGCAATTGAACCGGTGCCTAGAGA
AGGTGGCGCGGGGTAAACTGGGAAAGTGATGTCGTGTACTGGCTCCGCCTTTTTCCCGAGGGTGGGGGAGAACCGTATATAAGTG
CAGTAGTCGCCGTGAACGTTCTTTTTCGCAACGGGTTTGCCGCCAGAACACAGGTAAGTGCCGTGTGTGGTTCCCGCGGGCCTGGC
CTCTTTACGGGTTATGGCCCTTGCGTGCCTTGAATTACTTCCACCTGGCTGCAGTACGTGATTCTTGATCCCGAGCTTCGGGTTGGA
AGTGGGTGGGAGAGTTCGAGGCCTTGCGCTTAAGGAGCCCCTTCGCCTCGTGCTTGAGTTGAGGCCTGGCCTGGGCGCTGGGGCC
GCCGCGTGCGAATCTGGTGGCACCTTCGCGCCTGTCTCGCTGCTTTCGATAAGTCTCTAGCCATTTAAAATTTTTGATGACCTGCTG
CGACGCTTTTTTTCTGGCAAGATAGTCTTGTAAATGCGGGCCAAGATCTGCACACTGGTATTTCGGTTTTTGGGGCCGCGGGCGGC
GACGGGGCCCGTGCGTCCCAGCGCACATGTTCGGCGAGGCGGGGCCTGCGAGCGCGGCCACCGAGAATCGGACGGGGGTAGTCT
CAAGCTGGCCGGCCTGCTCTGGTGCCTGGCCTCGCGCCGCCGTGTATCGCCCCGCCCTGGGCGGCAAGGCTGGCCCGGTCGGCAC
CAGTTGCGTGAGCGGAAAGATGGCCGCTTCCCGGCCCTGCTGCAGGGAGCTCAAAATGGAGGACGCGGCGCTCGGGAGAGCGGG
CGGGTGAGTCACCCACACAAAGGAAAAGGGCCTTTCCGTCCTCAGCCGTCGCTTCATGTGACTCCACGGAGTACCGGGCGCCGTC
CAGGCACCTCGATTAGTTCTCGAGCTTTTGGAGTACGTCGTCTTTAGGTTGGGGGGAGGGGTTTTATGCGATGGAGTTTCCCCACA
CTGAGTGGGTGGAGACTGAAGTTAGGCCAGCTTGGCACTTGATGTAATTCTCCTTGGAATTTGCCCTTTTTGAGTTTGGATCTTGGT
TCATTCTCAAGCCTCAGACAGTGGTTCAAAGTTTTTTTCTTCCATTTCAGGTGTCGTGAGCGGCCGCTGAGTTAACTATTCTAGAGC
CACCATGGAGACGGACGTCTCAACaACTACCCCAGCACCGAGGCCACCCACCCCGGCTCCTACCATCGCCTCCCAGCCTCTGTCCC
TGCGTCCGGAGGCATGTAGACCCGCAGCTGGTGGGGCCGTGCATACCCGGGGTCTTGACTTCGCCTGCGATATCTACATTTGGGC
CCCTCTGGCTGGTACTTGCGGGGTCCTGCTGCTTTCACTCGTGATCACTCTTTACTGTAAGCGCGGTCGGAAGAAGCTGCTGTACA
TCTTTAAGCAACCCTTCATGAGGCCTGTGCAGACTACTCAAGAGGAGGACGGCTGTTCATGCCGGTTCCCAGAGGAGGAGGAAGG
CGGCTGCGAACTGCGCGTGAAATTCAGCCGCAGCGCAGATGCTCCAGCCTACCAACAGGGGCAGAACCAGCTCTACAACGAACT
CAATCTTGGTCGGAGAGAGGAGTACGACGTGCTGGACAAGCGGAGAGGACGGGACCCAGAAATGGGCGGGAAGCCGCGCAGAA
AGAATCCCCAAGAGGGCCTGTACAACGAGCTCCAAAAGGATAAGATGGCAGAAGCCTATAGCGAGATTGGTATGAAAGGGGAAC
GCAGAAGAGGCAAAGGCCACGACGGACTGTACCAGGGACTCAGCACCGCCACCAAGGACACCTATGACGCTCTTCACATGCAGG
CCCTGCCGCCTCGGTCCGGAGGCGGCGGAGAGGGCAGAGGAAGTCTTCTAACATGCGGTGACGTGGAGGAGAATCCCGGCCCTA
GGATGGTGAGCAAGGGCGAGGAGGATAACATGGCCATCATCAAGGAGTTCATGCGCTTCAAGGTGCACATGGAGGGCTCCGTGA
ACGGCCACGAGTTCGAGATCGAGGGCGAGGGCGAGGGCCGCCCCTACGAGGGCACCCAGACCGCCAAGCTGAAGGTGACCAAG
GGTGGCCCCCTGCCCTTCGCCTGGGACATCCTGTCCCCTCAGTTCATGTACGGCTCCAAGGCCTACGTGAAGCACCCCGCCGACAT
CCCCGACTACTTGAAGCTGTCCTTCCCCGAGGGCTTCAAGTGGGAGCGCGTGATGAACTTCGAGGACGGCGGCGTGGTGACCGTG
ACCCAGGACTCCTCCCTGCAGGACGGCGAGTTCATCTACAAGGTGAAGCTGCGCGGCACCAACTTCCCCTCCGACGGCCCCGTAA
TGCAGAAGAAGACCATGGGCTGGGAGGCCTCCTCCGAGCGGATGTACCCCGAGGACGGCGCCCTGAAGGGCGAGATCAAGCAGA
GGCTGAAGCTGAAGGACGGCGGCCACTACGACGCTGAGGTCAAGACCACCTACAAGGCCAAGAAGCCCGTGCAGCTGCCCGGCG
CCTACAACGTCAACATCAAGTTGGACATCACCTCCCACAACGAGGACTACACCATCGTGGAACAGTACGAACGCGCCGAGGGCC
GCCACTCCACCGGCGGCATGGACGAGCTGTACAAGTAAacgegtctggaacaatcaacctctggattacaaaatttgtgaaagattgactggtattct
taactatgttgctccttttacgctatgtggatacgctgctttaatgcctttgtatcatgctattgettcccgtatggctttcattttctcctccttgt
ataaatcctggttgctgtctctttatgaggagttgtggcccgttgtcaggcaacgtggcgtggtgtgcactgtgtttgctgacgcaacccccactggt
tggggcattgccaccacctgtcagctcctttccgggactttcgctttccccctccctattgccacggcggaactcatcgccgcctgccttgcccgctg
ctggacaggggctcggctgttgggcactgacaattccgtggtgttgtcggggaagctgacgtcctttccatggctgctcgcctgtgttgccacctg
gattctgcgcgggacgtccttctgctacgtcccttcggccctcaatccagcggaccttccttcccgcggcctgctgccggctctgcggcctcttcc
gcgtcttcgccttcgccctcagacgagtcggatctccctttgggccgcctccccgcctgaagtaaAGCGGCCGCATCGATACCGTCG
ACCTCGATCGAGACCTAGAAAAACATGGAGCAATCACAAGTAGCAATACAGCAGCTACCAATGCTGATTGTGCCTGGCTAGAAG
CACAAGAGGAGGAGGAGGTGGGTTTTCCAGTCACACCTCAGGTACCTTTAAGACCAATGACTTACAAGGCAGCTGTAGATCTTAG
CCACTTTTTAAAAGAAAAGGGGGGACTGGAAGGGCTAATTCACTCCCAACGAAGACAAGATATCCTTGATCTGTGGATCTACCAC
ACACAAGGCTACTTCCCTGATTGGCAGAACTACACACCAGGGCCAGGGATCAGATATCCACTGACCTTTGGATGGTGCTACAAGC
TAGTACCAGTTGAGCAAGAGAAGGTAGAAGAAGCCAATGAAGGAGAGAACACCCGCTTGTTACACCCTGTGAGCCTGCATGGGA
TGGATGACCCGGAGAGAGAAGTATTAGAGTGGAGGTTTGACAGCCGCCTAGCATTTCATCACATGGCCCGAGAGCTGCATCCGGA
CTGTACTGGGTCTCTCTGGTTAGACCAGATCTGAGCCTGGGAGCTCTCTGGCTAACTAGGGAACCCACTGCTTAAGCCTCAATAAA
GCTTGCCTTGAGTGCTTCAAGTAGTGTGTGCCCGTCTGTTGTGTGACTCTGGTAACTAGAGATCCCTCAGACCCTTTTAGTCAGTGT
GGAAAATCTCTAGCAGCATGTGAGCAAAAGGCCAGCAAAAGGCCAGGAACCGTAAAAAGGCCGCGTTGCTGGCGTTTTTCCATA
GGCTCCGCCCCCCTGACGAGCATCACAAAAATCGACGCTCAAGTCAGAGGTGGCGAAACCCGACAGGACTATAAAGATACCAGG
CGTTTCCCCCTGGAAGCTCCCTCGTGCGCTCTCCTGTTCCGACCCTGCCGCTTACCGGATACCTGTCCGCCTTTCTCCCTTCGGGAA
GCGTGGCGCTTTCTCATAGCTCACGCTGTAGGTATCTCAGTTCGGTGTAGGTCGTTCGCTCCAAGCTGGGCTGTGTGCACGAACCC
CCCGTTCAGCCCGACCGCTGCGCCTTATCCGGTAACTATCGTCTTGAGTCCAACCCGGTAAGACACGACTTATCGCCACTGGCAGC
AGCCACTGGTAACAGGATTAGCAGAGCGAGGTATGTAGGCGGTGCTACAGAGTTCTTGAAGTGGTGGCCTAACTACGGCTACACT
AGAAGAACAGTATTTGGTATCTGCGCTCTGCTGAAGCCAGTTACCTTCGGAAAAAGAGTTGGTAGCTCTTGATCCGGCAAACAAA
CCACCGCTGGTAGCGGTGGTTTTTTTGTTTGCAAGCAGCAGATTACGCGCAGAAAAAAAGGATCTCAAGAAGATCCTTTGATCTTT
TCTACGGGGTCTGACGCTCAGTGGAACGAAAACTCACGTTAAGGGATTTTGGTCATGAGATTATCAAAAAGGATCTTCACCTAGA
TCCTTTTAAATTAAAAATGAAGTTTTAAATCAATCTAAAGTATATATGAGTAAACTTGGTCTGACAGTTACCAATGCTTAATCAGT
GAGGCACCTATCTCAGCGATCTGTCTATTTCGTTCATCCATAGTTGCCTGACTCCCCGTCGTGTAGATAACTACGATACGGGAGGG
CTTACCATCTGGCCCCAGTGCTGCAATGATACCGCGAGACCCACGCTCACCGGCTCCAGATTTATCAGCAATAAACCAGCCAGCC
GGAAGGGCCGAGCGCAGAAGTGGTCCTGCAACTTTATCCGCCTCCATCCAGTCTATTAATTGTTGCCGGGAAGCTAGAGTAAGTA
GTTCGCCAGTTAATAGTTTGCGCAACGTTGTTGCCATTGCTACAGGCATCGTGGTGTCACGCTCGTCGTTTGGTATGGCTTCATTCA
GCTCCGGTTCCCAACGATCAAGGCGAGTTACATGATCCCCCATGTTGTGCAAAAAAGCGGTTAGCTCCTTCGGTCCTCCGATCGTT
GTCAGAAGTAAGTTGGCCGCAGTGTTATCACTCATGGTTATGGCAGCACTGCATAATTCTCTTACTGTCATGCCATCCGTAAGATG
CTTTTCTGTGACTGGTGAGTACTCAACCAAGTCATTCTGAGAATAGTGTATGCGGCGACCGAGTTGCTCTTGCCCGGCGTCAATAC
GGGATAATACCGCGCCACATAGCAGAACTTTAAAAGTGCTCATCATTGGAAAACGTTCTTCGGGGCGAAAACTCTCAAGGATCTT
ACCGCTGTTGAGATCCAGTTCGATGTAACCCACTCGTGCACCCAACTGATCTTCAGCATCTTTTACTTTCACCAGCGTTTCTGGGTG
AGCAAAAACAGGAAGGCAAAATGCCGCAAAAAAGGGAATAAGGGCGACACGGAAATGTTGAATACTCATACTCTTCCTTTTTCA
ATATTATTGAAGCATTTATCAGGGTTATTGTCTCATGAGCGGATACATATTTGAATGTATTTAGAAAAATAAACAAATAGGGGTTC
CGCGCACATTTCCCCGAAAAGTGCCACCTGAC
```

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

Further attributes, features, and embodiments of the present invention can be understood by reference to the following numbered aspects of the disclosed invention. Reference to disclosure in any of the preceding aspects is applicable to any preceding numbered aspect and to any combination of any number of preceding aspects, as recognized by appropriate antecedent disclosure in any combination of preceding aspects that can be made. The following numbered aspects are provided:

1. An engineered bifunctional receptor comprising an E3 ligase binding domain and a target binding domain operatively coupled to the E3 ligase binding domain.

2. The engineered bifunctional receptor of aspect 1, wherein the target binding domain is operatively coupled to the E3 ligase binding domain via a linker.

3. The engineered bifunctional receptor of aspect 2, wherein the linker is a flexible linker.

4. The engineered bifunctional receptor of any one of aspects 1-3, wherein the engineered bifunctional receptor inducibly binds an E3 ligase in response to a stimulus.

5. The engineered bifunctional receptor of aspect 4, wherein the stimulus is a chemical stimulus, a biological stimulus, or a physical stimulus.

6. The engineered bifunctional receptor of aspect 5, wherein the chemical stimulus is thalidomide or a derivative thereof.

7. The engineered bifunctional receptor of aspect 6, wherein the E3 ligase binding domain is or comprises a zinc finger domain.

8. The engineered bifunctional receptor of any one of aspects 4-7, further comprising a stimulus responsive domain.

9. The engineered bifunctional receptor of any one of aspects 1-8, wherein the E3 ligase binding domain is viral or eukaryotic.

10. The engineered bifunctional receptor of aspect 9, wherein the E3 ligase binding polypeptide is human.

11. The engineered bifunctional receptor of any one of aspects 1-10, wherein the engineered bifunctional receptor is configured for programmable and/or inducible degradation of one or more target polypeptides.

12. The engineered bifunctional receptor of any one of aspects 1-11, wherein the target binding domain is capable of binding one or more target polypeptides.

13. The engineered bifunctional receptor of any one of aspects 1-12, wherein the targeting binding domain is capable of binding two or more different target polypeptides.

14. The engineered bifunctional receptor substrate receptor of any one of aspects 1-13, wherein
a. the E3 ligase binding domain comprises or consists of a sequence according to SEQ ID NO: 78, 151, 152, 153, 154, 155, 156, 157, 158, 159, 160, or 161;
b. target binding domain comprises or consists of a sequence according SEQ ID NO: 38, 46, 48, 50, 76, 108, 110, 124, 132, 136, 138, 140, 145, 146, 147, 148, 149, 150; or
c. a combination of (a) and (b).

15. The engineered bifunctional receptor of any one of aspects 11-14, wherein the one or more target polypeptides or two or more different target polypeptides are bound by the target binding domain and are endogenously expressed in a cell-based therapeutic and are associated with decreasing or otherwise interfering with a therapeutic efficacy of the cell-based therapeutic.

16. The engineered bifunctional receptor of aspect 15, wherein the cell-based therapeutic is a chimeric antigen receptor (CAR) T cell, T cell receptor (TCR) T cells, tumour infiltrating lymphocyte, B-cell, a Natural Killer (NK) cell, a CAR-NK cell, a stem cell, or an induced pluripotent stem cell (iPSC).

17. The engineered bifunctional receptor of aspect 16, wherein the one or more target polypeptides or the two or more different target polypeptides mediate cellular dysfunction, exhaustion, transformation, or any combination thereof.

18. The engineered bifunctional receptor of any one of aspects 11-17, wherein the one or more target polypeptides or the two or more different target polypeptides are expressed in a cell used for biomanufacturing and are associated with decreasing a yield of a bioproduct or otherwise interfere with production of the bioproduct.

19. The engineered bifunctional receptor of any one of aspects 1-18, wherein the target binding domain comprises an antibody or fragment thereof, nanobody, affibody, knottin, DARPin, adnectin, anticalin, avimer, Fynomer, beta-hairpin mimetics, alphabody, or any combination thereof.

20. The engineered bifunctional receptor of any one of aspects 1-19, wherein the target binding domain comprises a protein receptor or receptor ligand.

21. An engineered polynucleotide comprising a polynucleotide that encodes the engineered bifunctional receptor of any one of aspects 1-20.

22. The engineered polynucleotide of aspect 21, further comprising a regulatory element operatively coupled to the polynucleotide that encodes the engineered bifunctional receptor.

23. The engineered polynucleotide of aspect 22, wherein the regulatory element is a constitutive promoter or an inducible promoter.

24. A vector or vector system comprising the engineered polynucleotide of any one of aspects 21-23.

25. A delivery vehicle comprising:
an engineered bifunctional receptor of any one of aspects 1-20, an engineered polynucleotide of any one of aspects 21-23, the vector or vector system of aspect 24, or any combination thereof.

26. A cell comprising:
(a) an engineered bifunctional receptor of any one of aspects 1-20, (b) an engineered polynucleotide of any one of aspects 21-23, (c) a vector or vector system of aspect 24, (d) a delivery vehicle of aspect 25, or any combination of (a)-(d).

27. The cell of aspect 26, wherein the cell is a cell for adoptive cell therapy.

28. The cell of aspect 27 wherein the cell is a chimeric antigen receptor (CAR) T cell, T cell receptor (TCR) T cells, tumor infiltrating lymphocyte, B-cell, or a Natural Killer cell.

29. A cell population comprising one or more cells of any one of aspects 26-28.

30. A pharmaceutical formulation comprising:
(a) an engineered bifunctional receptor of any one of aspects 1-20, (b) an engineered polynucleotide of any one of aspects 21-23, (c) a vector or vector system of aspect 24, (d) a delivery vehicle of aspect 25, (e) a cell of any one of aspects 26-28, (f) a cell population of aspect 29, or (g) any combination of (a)-(f); and
a pharmaceutically acceptable carrier.

31. A kit comprising:
(a) an engineered bifunctional receptor of any one of aspects 1-20, (b) an engineered polynucleotide of any one of aspects 21-23, (c) the vector or vector system of aspect 24, (d) the delivery vehicle of aspect 25, (e) a cell of any one of aspects 26-28, (f) a cell population of aspect 29, (g) a pharmaceutical formulation of aspect 30, or (h) any combination of (a)-(g).

32. A method of targeted protein degradation, the method comprising:
delivering (a) an engineered bifunctional receptor of any one of aspects 1-20, (b) an engineered polynucleotide of any one of aspects 21-23, (c) the vector or vector system of aspect 24, (d) the delivery vehicle of aspect 25, (e) a pharmaceutical formulation of aspect 30, or (f) any combination of (a)-(e) to a cell or cell population under conditions sufficient to permit binding of the target binding domain to one or more target polypeptides thereby triggering induced-proximity degradation of the one or more target polypeptides.

33. The method of aspect 32, wherein the cell or cell population has a decreased amount of the one or more target polypeptides as compared to a suitable control cell.

34. The method of any one of aspects 32-33, wherein the cell is a cell for adoptive cell therapy.

35. The method of claim 34, wherein the cell is a chimeric antigen receptor (CAR) T cell, T cell receptor (TCR) T cells, tumor infiltrating lymphocyte, B-cell, or a Natural Killer cell.

36. The method of any one of aspects 32-35, wherein the delivering occurs in vitro, in vivo, in situ, or ex vivo.

37. The method of any one of aspects 32-36, wherein the one or more target polypeptides are endogenous to the cell or cell population.

38. A method of treatment comprising:
delivering a cell or cell population as in any one of claims 26-29 to a subject in need thereof.

39. The method of aspect 38, wherein the cell or cell population is autologous or allogeneic.

40. The method of any one of aspects 32-39, further comprising delivering (a) an engineered bifunctional receptor of any one of aspects 1-20, (b) an engineered polynucleotide of any one of aspects 21-23, (c) a vector or vector system of aspect 24, (d) a delivery vehicle of aspect 25, (e) a pharmaceutical formulation of aspect 30, or (f) any combination of (a)-(e) to the cell or cell population under conditions sufficient to permit binding of the target binding domain to one or more target polypeptides thereby triggering induced-proximity degradation of the one or more target polypeptides.

41. The method of aspect 40, wherein delivering occurs ex vivo.

42. The method of any one of aspects 32-41, wherein the one or more target polypeptides are endogenous to the cell or cell population.

43. The method of any one of aspects 32-43, wherein the one or more target polypeptides mediate cellular dysfunction, exhaustion, transformation, or any combination thereof.

44. The method of any one of aspects 32-43, wherein the cell or cell population has a decreased amount of the one or more target polypeptides as compared to a suitable control cell.

45. The method of any one of aspects 32-44, wherein the subject in need thereof has a cancer.

1. An engineered bifunctional receptor comprising an E3 ligase binding domain and a target binding domain operatively coupled to the E3 ligase binding domain.

2. The engineered bifunctional receptor of claim 1, wherein:
the target binding domain is operatively coupled to the E3 ligase binding domain via a linker, optionally wherein the linker is a flexible linker:
the engineered bifunctional receptor inducibly binds an E3 ligase in response to a stimulus, optionally further comprising a stimulus responsive domain, optionally wherein the stimulus is a chemical stimulus, a biological stimulus, or a physical stimulus, optionally wherein the chemical stimulus is thalidomide or a derivative thereof, optionally wherein the E3 ligase binding domain is or comprises a zinc finger domain;
the E3 ligase binding domain is viral or eukaryotic, optionally wherein the E3 ligase binding domain is human;
the engineered bifunctional receptor is configured for programmable and/or inducible degradation of one or more target polypeptides;
the target binding domain is capable of binding one or more target polypeptides, optionally wherein the one or more target polypeptides are expressed in a cell used for biomanufacturing and are associated with decreasing a yield of a bioproduct or otherwise interfere with production of the bioproduct;
the targeting binding domain is capable of binding two or more different target polypeptides;
the target binding domain comprises an antibody or fragment thereof, nanobody, affibody, knottin, DARPin, adnectin, anticalin, avimer, Fynomer, beta-hairpin mimetics, alphabody, or any combination thereof; and/or
the target binding domain comprises a protein receptor or a receptor ligand.

3-13. (canceled)

14. The engineered bifunctional receptor of claim 1, wherein
a. the E3 ligase binding domain comprises or consists of a sequence according to SEQ ID NO: 78, 151, 152, 153, 154, 155, 156, 157, 158, 159, 160, or 161;
b. the target binding domain comprises or consists of a sequence according SEQ ID NO: 38, 46, 48, 50, 76, 108, 110, 124, 132, 136, 138, 140, 145, 146, 147, 148, 149, 150; or
c. a combination of (a) and (b).

15. The engineered bifunctional receptor of claim 14, wherein one or more target polypeptides are bound by the target binding domain and are endogenously expressed in a cell-based therapeutic and are associated with decreasing or otherwise interfering with a therapeutic efficacy of the cell-based therapeutic, optionally wherein the cell-based therapeutic is a chimeric antigen receptor (CAR) T cell, T cell receptor (TCR) T cells, tumour infiltrating lymphocyte, B-cell, a Natural Killer (NK) cell, a CAR-NK cell, a stem cell, or an induced pluripotent stem cell (iPSC), optionally wherein the one or more target polypeptides mediate cellular dysfunction, exhaustion, transformation, or any combination thereof.

16-20. (canceled)

21. An engineered polynucleotide comprising a polynucleotide that encodes the engineered bifunctional receptor of claim 1.

22. The engineered polynucleotide of claim 21, further comprising a regulatory element operatively coupled to the polynucleotide that encodes the engineered bifunctional receptor, optionally wherein the regulatory element is a constitutive promoter or an inducible promoter.

23. (canceled)

24. A vector or vector system comprising the engineered polynucleotide of claim 1.

25. A delivery vehicle comprising:
the engineered bifunctional receptor of claim 1, an engineered polynucleotide comprising a polynucleotide encoding the engineered bifunctional receptor, a vector or vector system comprising the engineered polynucleotide, or any combination thereof.

26. The delivery vehicle of claim 25, wherein the engineered polynucleotide further comprises a regulatory element operatively coupled to the polynucleotide encoding the engineered bifunctional receptor, optionally wherein the regulatory element is an inducible promoter.

27. (canceled)

28. A cell comprising:
(a) an engineered bifunctional receptor comprising an E3 ligase binding domain and a target binding domain operatively coupled to the E3 ligase binding domain, (b) an engineered polynucleotide comprising a polynucleotide encoding the engineered bifunctional receptor, (c) a vector or vector system comprising the engineered polynucleotide, (d) a delivery vehicle comprising (a), (b), (c) or any combination thereof, or any combination of (a)-(d).

29. The cell of claim 28, wherein:
the engineered polynucleotide further comprises a regulatory element operatively coupled to the polynucleotide encoding the engineered bifunctional receptor, optionally wherein the regulatory element is an inducible promoter;
the cell is a cell for adoptive cell therapy, optionally wherein the cell is a chimeric antigen receptor (CAR) T cell, T cell receptor (TCR) T cells, tumor infiltrating lymphocyte, B-cell, or a Natural Killer cell.

30-32. (canceled)

33. A cell population comprising one or more cells of claim 28.

34. The cell population of claim 33, wherein:
the engineered polynucleotide further comprises a regulatory element operatively coupled to the polynucleotide encoding the engineered bifunctional receptor, optionally wherein the regulatory element is an inducible promoter; and/or
the cell is a cell for adoptive cell therapy, optionally wherein the cell is a chimeric antigen receptor (CAR) T cell, T cell receptor (TCR) T cells, tumor infiltrating lymphocyte, B-cell, or a Natural Killer cell.

35-37. (canceled)

38. A pharmaceutical formulation comprising:
(a) the engineered bifunctional receptor of claim 1, (b) an engineered polynucleotide comprising a polynucleotide encoding the engineered bifunctional receptor, (c) a vector or vector system comprising the engineered polynucleotide, (d) a delivery vehicle comprising (a), (b), (c), or any combination of (a)-(c), (e) a cell comprising (a), (b), (c), (d), or any combination of (a)-(d), (f) a cell population comprising the cell, or (g) any combination of (a)-(f); and
a pharmaceutically acceptable carrier.

39. The pharmaceutical formulation of claim 38, wherein:
the engineered polynucleotide further comprises a regulatory element operatively coupled to the polynucleotide encoding the engineered bifunctional receptor, optionally wherein the regulatory element is an inducible promoter; and/or
the cell is a cell for adoptive cell therapy, optionally wherein the cell is a chimeric antigen receptor (CAR) T cell, T cell receptor (TCR) T cells, tumor infiltrating lymphocyte, B-cell, or a Natural Killer cell.

40-42. (canceled)

43. A kit comprising:
(a) an engineered bifunctional receptor comprising an E3 ligase binding domain and a target binding domain operatively coupled to the E3 ligase binding domain, (b) an engineered polynucleotide comprising a polynucleotide encoding the engineered bifunctional receptor, (c) a vector or vector system comprising the engineered polynucleotide, (d) a delivery vehicle comprising (a), (b), (c), or any combination of (a)-(c), a cell comprising (a), (b), (c), (d), or any combination of (a)-(d), (e) a cell comprising the cell, (f) a pharmaceutical formulation comprising any one of (a)-(e) or any combination thereof and a pharmaceutically acceptable carrier, or (h) any combination of (a)-(g).

44. A method of targeted protein degradation, the method comprising: delivering (a) the engineered bifunctional receptor of claim 1, (b) an engineered polynucleotide comprising a polynucleotide encoding the engineered bifunctional receptor, (c) a vector or vector system comprising the engineered polynucleotide, (d) a delivery vehicle comprising (a), (b), (c), or any combination of (a)-(c), (e) a pharmaceutical formulation comprising (a), (b), (c), (d), or any combination of (a)-(d), and a pharmaceutically acceptable carrier, or (f) any combination of (a)-(e) to a cell or cell population under conditions sufficient to permit binding of the target binding domain to one or more target polypeptides thereby triggering induced-proximity degradation of the one or more target polypeptides.

45. The method of claim 44, wherein:
the cell or cell population has a decreased amount of the one or more target polypeptides as compared to a suitable control cell, optionally wherein the cell or cell population is a cell for adoptive cell therapy, optionally wherein the cell is a chimeric antigen receptor (CAR) T cell, T cell receptor (TCR) T cells, tumor infiltrating lymphocyte, B-cell, or a Natural Killer cell;
the delivering occurs in vitro, in vivo, in situ, or ex vivo; and/or
the one or more target polypeptides are endogenous to the cell or cell population.

46-49. (canceled)

50. A method of treatment comprising:
delivering the cell of claim 28 to a subject in need thereof.

51. The method of claim 50:
wherein the cell or cell population is autologous or allogeneic;
further comprising delivering (a) the engineered bifunctional receptor of claim 1, (b) an engineered polynucleotide comprising a polynucleotide encoding the engineered bifunctional receptor, (c) a vector or vector system comprising the engineered polynucleotide, (d) a delivery vehicle comprising (a), (b), (c), or any combination of (a)-(c), (e) a pharmaceutical formulation comprising (a), (b), (c), (d), or any combination of (a)-(d), and a pharmaceutically acceptable carrier, or (f) any combination of (a)-(e) to the cell or cell population under conditions sufficient to permit binding of the target binding domain to one or more target polypeptides thereby triggering induced-proximity degradation of the one or more target polypeptides, optionally wherein: delivering occurs ex vivo: the one or more target polypeptides are endogenous to the cell or cell population: the one or more target polypeptides mediate cellular dysfunction, exhaustion, transformation, or any combination thereof; and/or the cell or cell population has a decreased amount of the one or more target polypeptides as compared to a suitable control cell; and/or
wherein the subject in need thereof has a cancer.

52-57. (canceled)
